(19) United States
(12) Patent Application Publication
(10) Pub. No.: US 2026/0347351 A1
(43) Pub. Date: Nov. 5, 2026

(54) INHIBITORS OF PDE11A4 AND METHODS OF USING SAME

(71) Applicants: UNIVERSITY OF MARYLAND, BALTIMORE, Baltimore, MD (US); MONTCLAIR STATE UNIVERSITY, Montclair, NJ (US)

(72) Inventors: Michele Patrice Kelly, Fulton, MD (US); David Rotella, Lake Hiawatha, NJ (US); Shams ul Mahmood, Cedar Grove, NJ (US)

(21) Appl. No.: 18/998,725

(22) PCT Filed: Jul. 28, 2023
(86) PCT No.: PCT/US2023/071289

§ 371 Date: Jan. 27, 2025

Related U.S. Application Data
(60) Provisional application No. 63/393,187, filed on Jul. 28, 2022.
(60) Provisional application No. 63/370,546, filed on Aug. 5, 2022.

(51) Int. Cl. C07D 471/04; A61K 31/437; A61P 25/28

(52) U.S. Cl. CPC C07D 471/04; A61K 31/437; A61P 25/28

(57) ABSTRACT

PDE11A4 inhibitors, and methods of using the same, are provided for treating or preventing diseases or disorder associated with cognitive decline.

### CROSS-REFERENCE TO RELATED APPLICATIONS

This application claims priority to and benefit of U.S. Provisional Patent Application Nos. 63/393,187, filed Jul. 28, 2022, and 63/370,546, filed Aug. 5, 2022, each of which are incorporated by reference herein in their entireties.

### STATEMENT AS TO FEDERALLY SPONSORED RESEARCH

This invention was made with government support under Grant Numbers AG061200, MH101130, and AG067836 awarded by the National Institutes of Health. The government has certain rights in the invention.

### FIELD

The disclosure relates generally to compounds that alter the subcellular location of PDE11A4 and/or inhibit the activity of PDE11A4, and methods of using such compounds as treatments for disease.

### BACKGROUND

After the age of 60, nearly all individuals experience some form of cognitive decline-particularly memory deficits- and no drugs are able to prevent or reverse this loss. Indeed, advanced age is the strongest risk factor for dementia. Even in absence of dementia, age-related cognitive impairment increases health care costs and risk for disability. Age-related cognitive decline is not a uniform process, with variability in symptom severity observed across individuals and across cognitive domains. Associative long-term memories (aLTMs)—particularly those involving experiences with family and friends—are more susceptible to age-related cognitive decline than are recognition long-term memories (rLTMs) for reasons that are not well understood. The lack of knowledge of the molecular mechanisms that govern age-related decline slows the development of novel therapeutics.

### SUMMARY

In aspects, the disclosure provides a compound of formula (A), or a pharmaceutically acceptable salt, solvate, hydrate, cocrystal, or prodrug thereof:

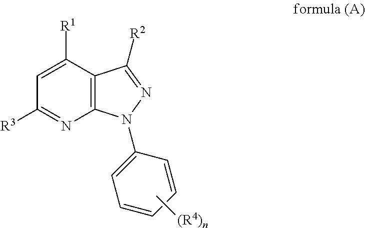

wherein in formula (A):
R¹ is selected from 5-membered heterocycle, 5-membered heteroaryl, 6-membered heterocycle, and 6-membered heteroaryl; or R¹ is

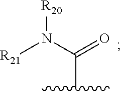

R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
R₂₀ is selected from H, optionally substituted alkyl, and optionally substituted haloalkyl;
R₂₁ is selected from optionally substituted alkyl and optionally substituted haloalkyl;
or R₂₀ and R₂₁ are joined to form a 4-7 membered ring with the nitrogen to which they are each bound;
n is an integer selected from 0-5; and
t is 1 or 2.

In embodiments, the compound of formula (A) is a compound of formula (I), or a pharmaceutically acceptable salt, solvate, hydrate, cocrystal, or prodrug thereof:

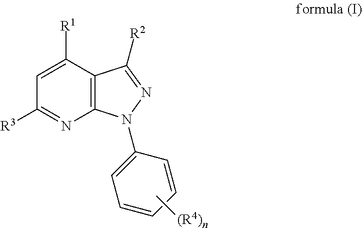

wherein in formula (I):
R¹ and R³ are independently at each occurrence a 5-membered heterocycle, 5-membered heteroaryl, a 6-membered heterocycle, or 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R)C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

In embodiments, R¹ is a 5-membered heteroaryl ring, optionally selected from thiazole, oxazole, imidazole, thiadiazole, oxadiazole, and triazole. In embodiments, the compound is a compound of formula (II):

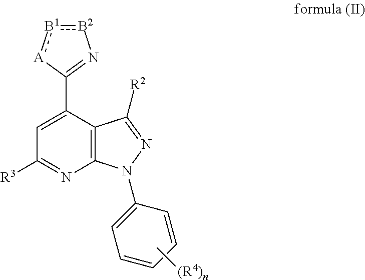

wherein in formula (II):
A is S, NR⁵, N, or O, optionally A is S or O;
B¹ and B² are independently at each occurrence selected from N, O, or CR⁵, wherein B¹ is not O when either A or B² is O; and
each R⁵ is independently at each occurrence selected from hydrogen, C₁-C₆ alkyl (e.g., C₁-C₃ alkyl), and C₁-C₃ haloalkyl (e.g., CH₂F, CHF₂, CF₃);
R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR, SR, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
n is an integer selected from 0-5; and
t is 1 or 2.

In embodiments, R¹ is selected from

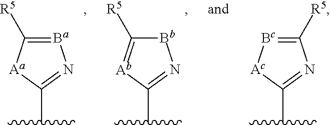

wherein:
A^{a} is O or S, and B^{a} is N or CH;
A^{b} is N, and B^{b} is O or S;
A^{c} is O or S, and B^{c} is N; and
R⁵ is selected from hydrogen, C₁-C₃ alkyl (e.g., methyl, ethyl, propyl, isopropyl), and C₁-C₃ haloalkyl (e.g., CH₂F, CHF₂, or CF₃).

In embodiments, R¹ is selected from

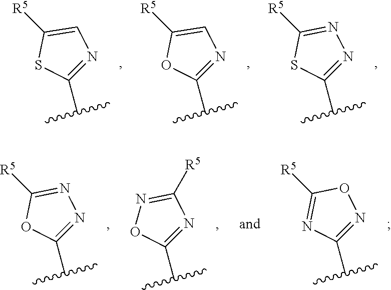

and R⁵ is selected from H, methyl, ethyl, propyl, isopropyl, CH₂F, and CHF₂. In embodiments, R¹ is selected from

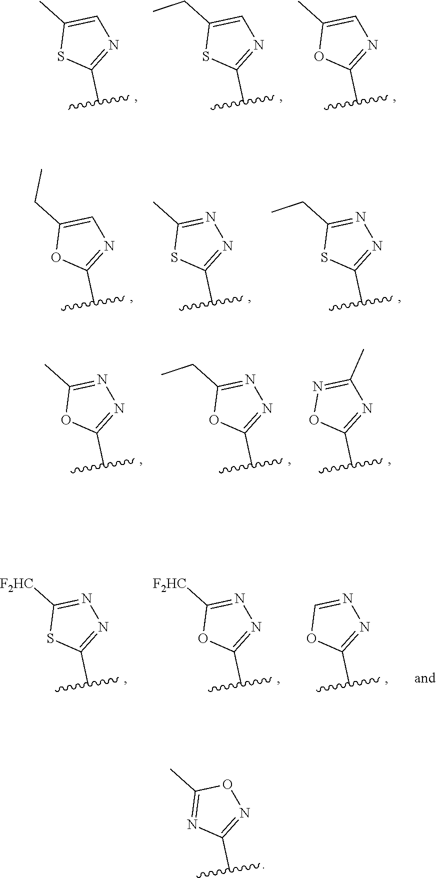

In embodiments, R₂ is selected from hydrogen, C₃-C₆ cycloalkyl, C₁-C₆ alkyl (e.g., C₁-C₄ straight chain alkyl, C₃-C₆ branched alkyl), —CF₃, —CH₂F, —CHF₂, and —C(R⁶R⁷)ₚX;
wherein, p is an integer from 1-3;
R⁶ and R⁷ are each independently selected from hydrogen, C₁-C₃ alkyl, and C₃-C₆ cycloalkyl;
X is selected from —OH, —CN, —OR⁸, and NR⁹R¹⁰;
R⁸ is selected from C₁-C₃ alkyl (e.g., C₁-C₃ straight or branched alkyl), and C₃-C₆ cycloalkyl; and
R⁹ and R¹⁰ are each independently selected from hydrogen, C₁-C₃ alkyl, and C₃-C₆ cycloalkyl.

In embodiments, R₂ is selected from hydrogen, C₁-C₄ alkyl (e.g., methyl, ethyl, propyl, isopropyl, butyl, tertbutyl), —CF₃, —CH₂F, and —CHF₂. In embodiments, R₂ is selected from H, —CH₃, —CF₃, and —CHF₂. In embodiments, R³ is a 5-membered heteroaryl ring, optionally selected from pyrrole, thiophene, furan, pyrazole, thiazole, isothiazole, oxazole, isoxazole, and imidazole. In embodiments, R³ is selected from

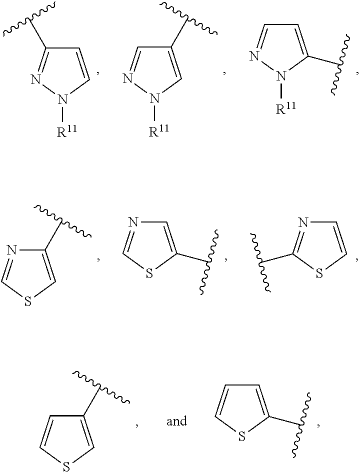

wherein R¹¹ is selected from hydrogen, C₁-C₃ alkyl (e.g., methyl, ethyl, propyl, isopropyl), C₃-C₆ cycloalkyl, CF₃, CH₂CF₃, CH₂CHF₂, and CH₂CH₂F. In embodiments, R³ is selected from

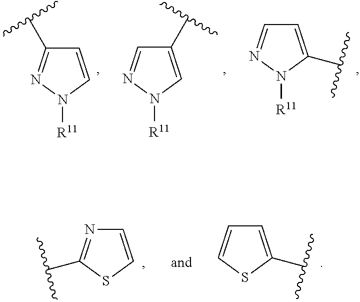

In embodiments, R¹¹ is selected from hydrogen, methyl, ethyl, propyl, isopropyl, —CF₃, and —CH₂CF₃. In embodiments, R³ is selected from

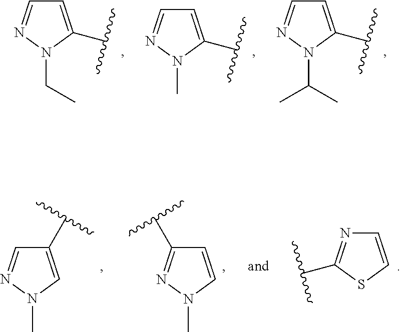

In embodiments, R³ is selected from

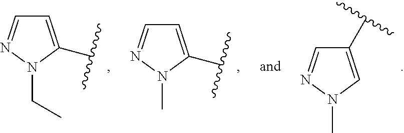

In embodiments, R⁴ is selected from hydrogen, C₁-C₆ alkyl, and halo (e.g., F or Cl). In embodiments, n is 0, 1, or 2. In embodiments, n is 0 or 1. In embodiments, R⁴ is selected from hydrogen, 4-fluoro, 3-fluoro, 2-fluoro, 2-chloro, and 4-chloro. In embodiments, R⁴ is selected from hydrogen, 4-fluoro, 2-fluoro, and 2-chloro. In embodiments, n is 2 and each R⁴ is 2-fluoro and 4-fluoro.

In embodiments, the compound of formula (A), formula (I), or formula (II) is a compound having any one of formula 1001-1080, 1097-1176, 1193-1272, 1289-1368, 1385-1729, 4001-4080, 4097-4176, 4193-4272, 4289-4368, 4385-4729, or a pharmaceutically acceptable salt, solvate, hydrate, cocrystal, or prodrug thereof. In embodiments, the compound of formula (A), formula (I), or formula (II) is a compound having any one of formula 2001-2337, or a pharmaceutically acceptable salt, solvate, hydrate, cocrystal, or prodrug thereof. In embodiments, the compound of formula (A), formula (I), or formula (II) is a compound having any one of the formula of Compounds 1013, 1014, 1109, 1110, 1015, 1625, 1111, 1011, 1009, 1107, 1105, 2217, 2218, 2008, 2210, 2315, 2219, 1682, 1691, 2017, 2015, 2016, 2087, 2019, 2001, 2020, 2021, 2222, 2215, 1448, 1543, 2264, 1393, 1717, 2278, 2027, 2041, 1103, 1101, or a pharmaceutically acceptable salt, solvate, hydrate, cocrystal, or prodrug thereof.

In embodiments, in the compound of formula (A), or pharmaceutically acceptable salt, solvate, hydrate, cocrystal, or prodrug thereof, R¹ is

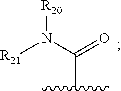

and R₂₀ is selected from H, optionally substituted C₁-C₆ alkyl, and optionally substituted C₁-C₆ haloalkyl; R₂₁ is selected from optionally substituted C₁-C₆ alkyl and optionally substituted C₁-C₆ haloalkyl; or R₂₀ and R₂₁ are joined to form a 4-6-membered heterocycle with the nitrogen to which they are each bound. In embodiments, R₂₀ is selected from H, methyl, ethyl, propyl, isopropyl, butyl, tert-butyl, CF₃, CHF₂, CH₂F, CH₂CF₃, CH₂CHF₂, and CH₂CH₂F; and R₂₁ is selected from methyl, ethyl, propyl, isopropyl, butyl, tert-butyl, CF₃, CHF₂, CH₂F, CH₂CF₃, CH₂CHF₂, and CH₂CH₂F; or R₂₀ and R₂₁ are joined to form a 4-6-membered heterocycle with the nitrogen to which they are each bound. In embodiments, R₂₀ is selected from H, methyl, ethyl, propyl, tert-butyl, and CH₂CH₂F; and R₂₁ is selected from methyl, ethyl, propyl, tert-butyl, and CH₂CH₂F; or R₂₀ and R₂₁ are joined to form a 4-6-membered heterocycle with the nitrogen to which they are each bound.

In embodiments, the compound of formula (A), pharmaceutically acceptable salt, solvate, hydrate, cocrystal, or prodrug thereof, is a compound of formula (III):

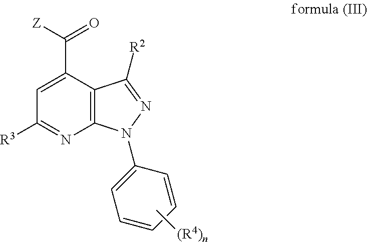

wherein in formula (III):
Z is selected from

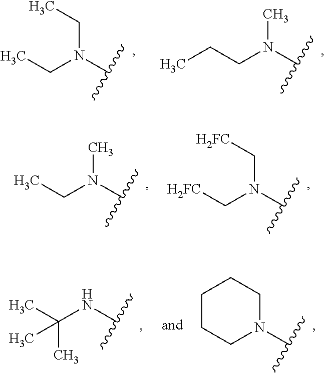

R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

In embodiments, R₂ is selected from hydrogen, C₃-C₆ cycloalkyl, C₁-C₆ alkyl (e.g., C₁-C₄ straight chain alkyl, C₃-C₆ branched alkyl), —CF₃, —CH₂F, —CHF₂, and —C(R⁶R⁷)ₚX; wherein, p is an integer from 1-3; R⁶ and R⁷ are each independently selected from hydrogen, C₁-C₃ alkyl, and C₃-C₆ cycloalkyl; X is selected from —OH, —CN, —OR⁸, and NR⁹R¹⁰; R⁸ is selected from C₁-C₃ alkyl (e.g., C₁-C₃ straight or branched alkyl), and C₃-C₆ cycloalkyl; and R⁹ and R¹⁰ are each independently selected from hydrogen, C₁-C₃ alkyl, and C₃-C₆ cycloalkyl. In embodiments, R₂ is selected from hydrogen, C₁-C₃ alkyl (e.g., methyl, ethyl, propyl, isopropyl), —CF₃, —CH₂F, and —CHF₂. In embodiments, R₂ is selected from hydrogen and —CH₃.

In embodiments, R³ is a 5-membered heteroaryl ring, optionally selected from pyrrole, thiophene, furan, pyrazole, thiazole, isothiazole, oxazole, isoxazole, and imidazole. In embodiments, R³ is selected from

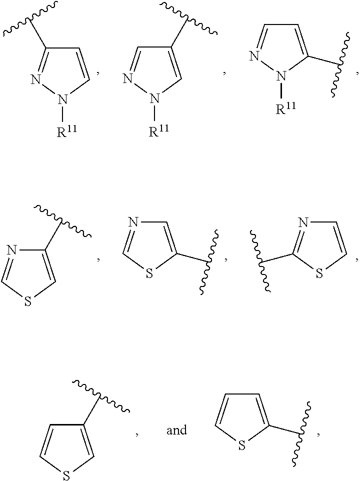

wherein R¹¹ is selected from hydrogen, C₁-C₃ alkyl (e.g. methyl, ethyl, propyl, isopropyl), C₃-C₆ cycloalkyl, CF₃, CH₂CF₃, CH₂CHF₂, and CH₂CH₂F. In embodiments, R³ is selected from

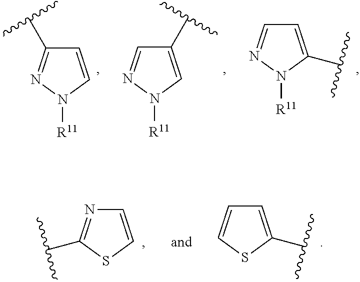

In embodiments, R¹¹ is selected from hydrogen, methyl, ethyl, propyl, isopropyl, —CF₃, and —CH₂CF₃. In embodiments, R³ is selected from

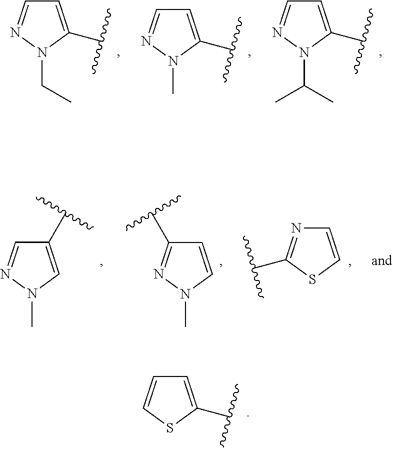

In embodiments, R⁴ is selected from hydrogen, C₁-C₆ alkyl, and halo (e.g., F or Cl). In embodiments, n is 0 or 1. In embodiments, R⁴ is selected from hydrogen, 4-fluoro, 3-fluoro, 2-fluoro, 2-chloro, and 4-chloro. In embodiments, R⁴ is selected from hydrogen, 4-fluoro, 3-fluoro, 2-fluoro, and 4-chloro.

In embodiments, the compound of formula (A) or formula (III) is a compound having any one of formula 3001, 3006-3021, 3023-3031, or a pharmaceutically acceptable salt, solvate, hydrate, cocrystal, or prodrug thereof.

In some embodiments, a compound of the disclosure is a PDE11A4 inhibitor. In some embodiments, the PDE11A4 inhibitor is a PDE11A4 selective inhibitor.

The present disclosure provides pharmaceutical compositions comprising a compound of the disclosure, or a pharmaceutically acceptable salt, solvate, hydrate, cocrystal, or prodrug thereof, and a physiologically compatible carrier medium. The present disclosure provides a pharmaceutical composition comprising a compound of the disclosure, or a pharmaceutically acceptable salt, solvate, hydrate, cocrystal, or prodrug thereof, and a physiologically compatible carrier medium, wherein the amount of the compound in the composition is a therapeutically effective amount for the treatment or prevention of a disease or disorder alleviated by inhibiting PDE11A4 activity in a patient in need thereof. In various embodiments, the disease or disorder is associated with cognitive decline. In some embodiments, the disease or disorder is dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND); or cognitive decline associated with spatial memory, other depression-related behaviors, additional anxiety-related behaviors, sensorimotor gating, or social behaviors.

The present disclosure provides methods of treating or preventing a disease or disorder alleviated by inhibiting PDE11A4 activity in a patient in need thereof, comprising administering to the patient a therapeutically effective amount of a compound of the disclosure. The present disclosure provides methods of treating or preventing a disease or disorder alleviated by inhibiting PDE11A4 activity in a patient in need thereof, comprising administering to the patient a therapeutically effective amount of a pharmaceutical composition of the disclosure. In some embodiments, the method includes administering the compound in a dosage unit form. In some embodiments, the dosage unit comprises a physiologically compatible carrier medium. In some embodiments, the method comprises treating or preventing a disease or disorder that is associated with cognitive decline. In some embodiments, the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

These and other embodiments, features, and potential advantages will become apparent with reference to the following description and drawings.

### BRIEF DESCRIPTION OF THE DRAWINGS

The foregoing summary, as well as the following detailed description of embodiments of the disclosure, are better understood when read in conjunction with the appended drawings and figures.

FIG. 1. is an image showing how PDE11A4 mRNA expression is restricted to the HIPP. Protein is expressed in neuronal cell bodies, dendrites, and axons.

FIGS. 2A-2C illustrate increases in hippocampal PDE11A expression. FIG. 2A is a graph of experimental data showing how hippocampal PDE11A expression is increased in old vs. young mice. FIG. 2B. is a graph of experimental data showing how hippocampal PDE11A expression is increased in adult (18-40 yrs) vs. prenatal humans. FIG. 2C is a graph of experimental data showing how hippocampal PDE11A expression is increased in demented vs. non-demented aged humans (>75 yrs) with a history of TBL Post hoc *vs. Young, prenatal, or ‘No’ group, P<0.05. A.U. arbitrary units.

FIGS. 3A-3C illustrate aging preferentially impairs aLTMs in mice. FIG. 3A is a graph of experimental data showing impaired aLTM for social transmission of food preference (STFP; n=25-34/group) 7 days after training. FIG. 3B is a graph of experimental data illustrating male and female aged C57BL/6J mice intact rLTM 7 days after training as measured by social odor recognition (SOR; n=15-16/age). FIG. 3C is a graph of experimental data illustrating non-social odor recognition (NSOR; n=7-8/age) in old and young mice. Memory refers to eating significantly more of the trained vs. novel food (FIG. 3A) or investigating the novel odor longer than the familiar odor (FIG. 3A, 3B). Young=2-6 months; Old=17-22 months. Post hoc *vs. familiar odor or novel food, P<0.01; #vs. Young, P=0.019.

FIG. 4A is a graph of experimental data showing male and female old PDE11A WT mice (WT-0) having no memory for STFP 7 days after training; however old PDE11A KO mice (KO-0) and heterozygous mice show robust memory equivalent to that of young (Y) mice. The protective effect of PDE11A deletion was replicated across sexes in 2 large cohorts, the combined analysis of which is shown here (n=26-29/group). The protective effect of PDE11A deletion on aLTMs is specific. FIG. 4B is a graph of experimental data showing there is no difference in rLTM between WT-O and KO-O mice, as measured in non-social odor recognition (NSOR; n=14). Young WTs were not included in this pilot study since aging does not impair NSOR—see FIG. 3.]. Y=5-7 months; 0=18-21 months. Post hoc *vs. novel food or familiar odor, P<0.001; #vs. WT-0, P<0.001.

FIG. 5A is an image of non-limiting example that due to the large size of PDE11A4 (>3 kb), a lentivirus was used to overexpress a GFP-mPDE11A4 fusion or GFP alone (negative control).

FIG. 5B shows western blots of DHIPP and VHIPP that illustrate titrating virus delivery allows overexpression of PDE11A4 in a dorsal<ventral gradient, as is seen in vivo.

FIG. 5C is a graph of experimental data showing how virally-expressed PDE11A4 engages relevant signal transduction cascades as it restores phosphorylation of the ribosomal protein S6 specifically at residues 235/236 (n=4-5/group), which was previously showed is significantly reduced in the hippocampus of KO vs WT.

FIG. 5D is a graph of experimental data illustrating mice trained on STFP and tested 7 days after training. KO mice expressing GFP in the hippocampus show strong aLTM for STFP; however, KO mice overexpressing PDE11A4 in the hippocampus (that is, mimicking the state of an “old WT”) fail to show significant aLTM for STFP.

FIG. 5E is a graph of experimental data illustrating the ability of PDE11A4 overexpression to impair aLTM, as KO mice treated with either the GFP or PDE11A4 lentivirus show strong rLTM for NSOR.

FIG. 6 is a graph of experimental data demonstrating that compound 25b was more potent and more efficacious than the drug tadalafil in reversing aging-like PDE11A4 phenotype.

FIG. 7A is an image and graph of experimental data illustrating phosphomimic mutations of PDE11A4 at serines 117 and 124 (117D124D) synergize, increasing the accumulation of PDE11A4 in distinct structures. Phosphoresistant mutations S117AS124A have the opposite effect.

FIG. 7B is a graph and image showing how biochemical fractionation of S 117DS124 shifts PDE11A4 from the cytosol to the membrane.

FIG. 7C is a graph of experimental data and image showing how S117 and S124 also synergize at the level of phosphorylation.

FIG. 7D is a graph of experimental data and image showing that disrupting PDE11A4 homodimerization using an isolated GAF-B (GB) domain decreases pS117 (P=0.07) and pS124.

FIG. 7E is a graph of experimental data normalizing the hyperaccumulation that is seen with S117D124D.

FIG. 7F is a graph of experimental data and image showing expression of St 17D124D mimics age-related decreases in cGMP levels.

FIG. 7G is a graph of experimental data and image showing how expression of the isolated GAF-B domain has the opposite effect as what is shown in FIG. 7F.

FIG. 8. is an image showing immuno-fluorescence with a total PDE11A antibody (top-green) and pS 117/pS124-PDE11A4 antibody (bottom-green) suggest that age-related increases in PDE11A expression occur in a compartment-specific manner. KOs virally overexpressing PDE11AWT show the same pattern as “old”.

FIG. 9 is a graph of experimental data and image showing age-related increases in VHIPP PDE11A4 protein expression occurs preferentially in membrane fractions, consistent with the in vitro studies showing S117D/S124D mutations shift PDE11A4 from the cytosol to the membrane.

FIG. 10 is an image showing how mPde11a4-mCherry reports transcriptional activity in ventral CAT (vCA1) but not ventral dentate gyrus (vDG), consistent with endogenous expression pattern of mPde11a4 (see also FIG. 1). In contrast, the ubiquitous Pgk-mCherry construct reports transcription in both subregions.

FIG. 11 is a graph of experimental data showing how p54nrb/NONO and XRN2 mRNA expression are reduced with age in human hippocampus. These reductions significantly correlate with the age-related increases in PDE11A mRNA expression shown in FIG. 2B (p54nrb/NONO: r=−0.424, P=0.016; XRN2: r=−0.352, P=0.048).

FIG. 12A is an image showing widefield fluorescence's ability to label PDE11A4 mRNA (red) using RNAscope. A confocal microscope in the USC IRF are used to collect z-stack images.

FIG. 12B is an image showing an example of subcellular resolution provided by confocal microscope. Shown is PDE11A4 protein (green) in COS1 cells counterstained for the nuclear marker DAPI (blue). Optical sections through the cell (shown to the right and bottom) clarify whether labeling observed from above is in the nucleus or cytosol.

FIG. 13 is a flowchart illustrating a non-limiting example of a pathway for pharmacologic inhibition of PDE11A.

FIGS. 14A-14C show increases in hippocampal PDE11A expression. FIG. 14A is a graph of experimental data showing how hippocampal PDE11A expression is increased in Old vs. young mice. FIG. 14B is a graph of experimental data showing how hippocampal PDE11A expression is increased in adult (18-40 yrs) vs prenatal humans. FIG. 14C is a graph of experimental data showing how hippocampal PDE11A expression is increased in demented vs. non-demented aged humans (>75 years) with a history of TBI. Post hoc *vs. Young, prenatal, or “no” group, P<0.05. A.U. —arbitrary units.

FIGS. 15A-15C show aging preferentially impairs aLTMs in mice. FIG. 15A is a graph of experimental data showing how aging preferentially impairs aLTMs in mice, relative to young mice (Y), male and female old C57BL/6J mice (O) show impaired remote aLTM for social transmission of food preference (STFP; n=25-34/group) 7 days after training. FIG. 15B is a graph of experimental data showing how aging of male and female c57BL/6J mice do not show significantly impaired rLTM as measured by social odor recognition (SOR; n=15-16/age). FIG. 15C is a graph of experimental data showing how aging affects non-social odor recognition (NSOR; n=7-8/age) 7 days after training. Young=2-5 months; old=17-22 months. Post hoc *significantly >0 (=memory), P<0.01; #vs. Young, P=0.019.

FIG. 16A is a graph of experimental data showing old PDE11A WT mice (WT-0) show no memory for STFP 7 days after training.; old PDE11A KO mice (KO-O) and heterogeneous mice (HT-0) show robust memory equivalent to that of young (Y) mice. FIG. 16B is a graph of experimental data showing there is no difference in rLTM between WT-O and KO-O mice, as measured in non-social odor recognition (NSOR; n=14). Y=5-7 months; O=18-21 months. Post hoc *significantly >0 (=memory); #vs. WT-Y, P<0.001; @vs. WT-O, P<0.003.

FIGS. 17A-17C show disrupting PDE114A expression in old WT mice rescues age-related impairments in aLTM for STFP. FIG. 17A is a graph of experimental data showing in vitro expression of an isolated GAF-B domain peptide (GB) disrupts PDE11A4 homodimerization and impairs PDE11A4 cGMP-hydrolytic activity relative to expression of an mCherry negative control (mCh). FIG. 17B is a graph of experimental data showing viral expression of the GB peptide in dorsal and ventral hippocampus of old PDE11A wild-type mice reverses age-related impairments in aLTM for STFP. FIG. 17C is a graph of experimental data showing GB had no effect on NSOR.

FIG. 18A is a graph of experimental data showing the results of COS1 monkey fibroblast cells transiently transfected with mouse PDE11A (95% homologous to human PDE11A4) used to confirm inhibition in a mammalian context and assess effects on subcellular compartmentalization.

FIG. 18B Is a graph showing cGMP relative to the negative control GFP. The assay can detect functionally-selective changes from baseline activity, as shown using phosphomimic mutations at serines 117, 124, and 162.

FIG. 18C is an image showing immunofluorescence with a total PDE11A antibody (top-green; blue signal=nuclear stain) and pS 118/pS124-PDE11A4 antibody (bottom-green).

FIG. 18D is a graph illustrating how age-related increases in PPDE11A expression lead to increased PPDE11A4 phosphorylation that causes formation of PPDE11A4 aggregates in the HIPP.

FIG. 18E is an image showing COS1 cells used to model the aggregation.

FIG. 18F is a graph of experimental data illustrating how manipulations that increase or decrease the aggregation. The GAF-B construct that reverses age-related memory impairments blocks this aggregation in COS1 cells.

FIG. 19 shows an image of chemical formulas of amine examples 1-11.

FIG. 20 shows an image of chemical formulas amine examples 12-16.

FIG. 21 shows an image of chemical formulas amine examples 17-19.

FIG. 22 shows a non-limiting process of in vivo screening of young and old C57BL6 mice. Y, young; O, old, V, vehicle; D1-3, dose 1-3.

FIG. 23 is an image showing a non-limiting example of a scheme of a synthesis of compounds 5 (a-s).

FIG. 24 is an image showing a non-limiting example of a scheme to prepare compounds 10 (a-b).

FIG. 25 is an image showing a non-limiting example of a scheme to prepare compounds 12 (a-b).

FIG. 26A is an image of exemplary PDE11A inhibitors of the disclosure. FIG. 26B is an image and a table illustrating PDE11A inhibition activity of exemplary compounds of the disclosure.

FIG. 27 is an image of exemplary PDE11A inhibitors of the disclosure.

FIG. 28 is an image of exemplary PDE11A inhibitors of the disclosure.

FIG. 29 is an image of HT22 cells overexpressing EmGFP-mPDE11A4.

FIGS. 30A-31B is an image of HT-22 cells overexpressing EmGFP-mPDE11A4 treated with the vehicle DMSO (FIG. 31A) or 1 uM 25b (FIG. 31B).

### DEFINITIONS

Unless defined otherwise, all technical and scientific terms used herein have the same meaning as is commonly understood by one of skill in the art to which this disclosure belongs. All patents and publications referred to herein are incorporated by reference in their entireties.

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include the polypeptides, polynucleotides, and compositions described herein. The terms “active pharmaceutical ingredient” and “drug” may also include those compounds described herein that bind PDE11A4 and thereby modulate (e.g. inhibit) PDE11A4 activity.

The term “isostere” refers to a group or molecule whose chemical and/or physical properties are similar to those of another group or molecule. A “bioisostere” is a type of isostere and refers to a group or molecule whose biological properties are similar to those of another group or molecule. For example, for the PDE11A4 inhibitors described herein, a carboxylic acid may be replaced by one of the following bioisosteres for carboxylic acids, including, without limitation, alkyl esters (COOR), acylsulfonamides (CONR—SO₂R), hydroxamic acids (CONR—OH), hydroxamates (CONR—OR), tetrazoles, hydroxyisoxazoles, isoxazol-3-ones, and sulfonamides (SO₂NR), where each R may independently represent hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that induces a particular response in target cells (e.g., the reduction of platelet adhesion and/or cell migration). The specific dose varies depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition). As used herein, the terms “prevent,” “preventing,” and/or “prevention” may refer to reducing the risk of developing a disease, disorder, or pathological condition.

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. For example, the compounds described herein may modulate (e.g., inhibit) PDE11A4 protein. In some embodiments, the compounds described herein may selectively modulate (e.g., selectively inhibit) PDE11A4 protein as compared to other PDE11A proteins. In some embodiments, the compounds described herein may selectively modulate (e.g., selectively inhibit) PDE11A4 protein as compared to other PDE or PDE11A proteins. “Modulate” and “modulation” also include changing the subcellular localization and/or location of PDE11A4. “Modulate” and “modulation” also include disrupting and/or preventing homodimerization of PDE11A4. “Modulate” and “modulation” also include direct modulation of PDE11A4 (e.g. modulation of catalytic activity of PDE11A4). “Modulate” and “modulation” also include indirect modulation of PDE11A4 (e.g. disrupting and/or preventing homodimerization of PDE11A4). “Inhibit” and “inhibiting” also include changing the subcellular localization and/or location of PDE11A4. “Inhibit” and “inhibiting” also include disrupting and/or preventing homodimerization of PDE11A4. “Inhibit” and “inhibiting” also include direct inhibition of PDE11A4 (e.g. inhibition of catalytic activity of PDE11A4). “Inhibit” and “inhibiting” also include indirect inhibition of PDE11A4 (e.g. disrupting and/or preventing homodimerization of PDE11A4).

The terms “QD,” “qd,” or “q.d.” mean quaque die, once a day, or once daily. The terms “BID,” “bid,” or “b.i.d.” mean his in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts is formed with inorganic acids and organic acids. Preferred inorganic acids from which salts is derived include, for example, hydrochloric acid, hydrobromic acid, sulfuric acid, nitric acid and phosphoric acid. Preferred organic acids from which salts is derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts is formed with inorganic and organic bases. Inorganic bases from which salts is derived include, for example, sodium, potassium, lithium, ammonium, calcium, magnesium, iron, zinc, copper, manganese and aluminum. Organic bases from which salts is derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” or “physiologically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

A “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a compound of any one of formula (I), formula (10), formula (II), formula (20), formula (21), formula (22), formula (23), formula (24), formula (25), formula (26), formula (27), formulas 1001-1144 and 2001-2032. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, omithine and methionine sulfone. Additional types of prodrugs are also encompassed. For instance, free carboxyl groups is derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that is converted in vivo to provide the bioactive agent (e.g., a compound of any one of formula (I), formula (10), formula (II), formula (20), formula (21), formula (22), formula (23), formula (24), formula (25), formula (26), formula (27), formulas 1001-1144 and 2001-2032) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g., organ or tumor-targeting, lymphocyte targeting), to modify or improve aqueous solubility of a drug (e.g., i.v. preparations and eyedrops), to improve topical drug delivery (e.g., dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range, e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (nPr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclic radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2). —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Acylsulfonamide” refers to the group —C(═O)NR^{a}—S(═O)₂R^{a}, where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Carboxaldehyde” refers to a —(C═O)H radical.

“Carbonyl” refers to the group —C(═O)—. Carbonyl groups may be substituted with the following exemplary substituents: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —NR^{a}—OR^{a}—, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e., (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, acylsulfonamido, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, acylsulfonamido, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}-(where t is 1 or 2), —OC(O)—R^{a}, —N(R^{a})₂, —C(O)R, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂. —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they is combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R³)₂, —C(O)R^{a}, —C(O)OR³, —OC(O)N(R^{a})₂, —C(O)N(R)₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides is prepared by treatment of the corresponding amino group with, for example, hydrogen peroxide or m-chloroperoxybenzoic acid.

“Amide” or “amido” refers to a chemical moiety with formula —C(O)NR^{a}R^{b} or —NR^{a}C(O)R^{b}, where R^{a} and R^{b} are selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R^{a} and R^{b} of —C(O)N R^{a}R amide may optionally be taken together with the nitrogen to which they are attached to form a 4-, 5-, 6- or 7-membered ring. Unless stated otherwise specifically in the specification, an amido group is optionally substituted independently by one or more of the substituents as described herein for alkyl, amino, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal stheces such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal stheces such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, acylsulfonamido, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkvlheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkvlheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkvlcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, isoxazol-3-one, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Hydroxamate” refers to the —C(O)NR^{a}OR^{a} moiety, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon is specified by either (R) or (S). Resolved compounds whose absolute configuration is unknown is designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that is defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers is prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound is determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers is isolated from mixtures by methods known to those skilled in the art, including chiral high pressure liquid chromatography (HPLC) and the formation and crystallization of chiral salts; or preferred enantiomers is prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment is significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers is reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction is carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, 3rd Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxamate, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(02)-(optionally substituted alkyl), —S(02)-(optionally substituted amino), —S(02)-(optionally substituted aryl), —S(02)-(optionally substituted heteroaryl), and —S(02)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂₀H radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### DETAILED DESCRIPTION

### Inhibition of PDE11A4

An enzyme called phosphodiesterase 11A (PDE11A) and its role in the neurobiological substrates of memory and social behaviors has been examined. PDE11A is a member of the large phosphodiesterase enzyme family and was originally cloned in 2000. The enzyme is derived from a single gene product, hydrolyzes both cAMP and cGMP and exists in 4 isoforms. The enzyme is predominantly expressed in the brain. In particular, PDE11A is found in the anterior hippocampus and more specifically in neurons in the superficial layer of CAT, the subiculum and the amygdalohippocampal area of the hippocampus. Moreover, PDE11A4 is the only PDE whose expression in brain emanates from the hippocampus, a region of the brain associated with associated long term memory (aLTM). Little is known of the signaling pathways lying up or downstream of PDE11A4, but it has been shown that PDE11A appears to regulate important signals for memory consolidation, including glutamatergic and calcium/calmodulin-dependent kinase II (CamKII) signaling, as well as protein synthesis. It has been shown that cAMP and cGMP signaling are decreased in aged and demented hippocampus in rats and humans. These age-related decreases in cyclic nucleotides are associated with increased expression of PDE11A4 in rodents and in humans with hippocampal dementia, versus non-demented aged subjects with a history of traumatic brain injury.

PDE11A4 is a dual acting cyclic nucleotide hydrolase expressed in neurons in the CAT, subiculum, amygdalostriatal transition area, and amygdalohippocampal area of the extended hippocampal formation. PDE11A4 is the only PDE enzyme to emanate solely from the hippocampal formation, a key brain region for the formation of long-term memory. PDE11A4 expression increases in the hippocampal formation of both humans and rodents as they age. PDE11A knockout mice do not show age-related deficits in associative memory and show no gross histopathology. While not wanting to be bound by any particular theory, this suggests inhibition of PDE11A4 can be useful as a therapeutic option for age-related cognitive decline.

In aspects, the present disclosure discloses novel PDE11A4 inhibitors with improved potency, including more than ten-fold improvement in potency compared to tadalafil in cell based activities, and/or improvements in pharmaceutical properties including selectivity and cell-penetrant PDE11A4 inhibitors.

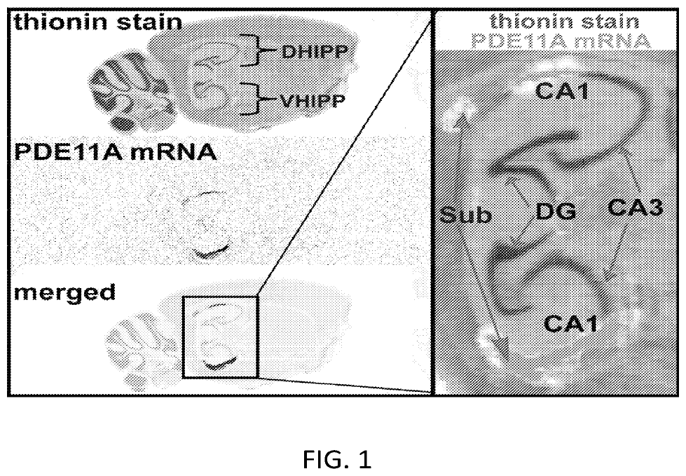

The PDE11A family, which breaks down cAMP and cGMP, is comprised of a single gene that is spliced into 4 isoforms, PDE11A1 through PDE11A4. The longest isoform, PDE11A4, is the isoform that is expressed in brain and it is ~95% homologous across mouse, rat and human. This high degree of homology argues that the results obtained in rodent models translates across species. PDE11A single nucleotide polymorphisms (SNPs) have been associated with major depressive disorder (MDD), suicide risk, antidepressant response in patients with MDD, and lithium response in patients with bipolar disorder. Both MDD and BPD have been conceptualized as diseases of accelerated aging. It was established that PDE11A4 is expressed in the brain. PDE11A4 was found in brain because PDE11A knockout (KO) mice was phenotyped and what few phenotypes they had were found to be of relevance to ventral hippocampal function. Thus, the search for a PDE11A isoform in brain was directed to the ventral hippocampal formation (VHIPP) to detect expression restricted to this small brain region. Indeed, it was discovered that PDE11A4 was strongly expressed in neurons of the superficial layer of CA1, the subiculum, and the adjacently connected amygdalohippocampal area (AHi) of the VHIPP (FIG. 1A), with little expression in dorsal HIPP (DHIPP) and no expression in other brain regions or over 20 peripheral organs. Only the nervous system shows a specific PDE11A4 signal. This makes PDE11A4 very unique because in brain it is the only PDE to emanate only from the HIPP, a structure critical for social aLTMs. This enrichment of PDE11A4 in the HIPP has now been independently confirmed by multiple investigators. This-along with the fact that PDE11A is a highly druggable enzyme-makes PDE11A a very attractive drug target because it stands to selectively restore aberrant cyclic nucleotide signaling in a brain region affected by age-related decline without directly affecting signaling in other brain regions or peripheral organs that might lead to unwanted side effects. At the very least, PDE11A4 molecularly defines an exceptionally discrete circuit within a brain region key to learning and memory, making it ripe for the study of age-related cognitive decline.

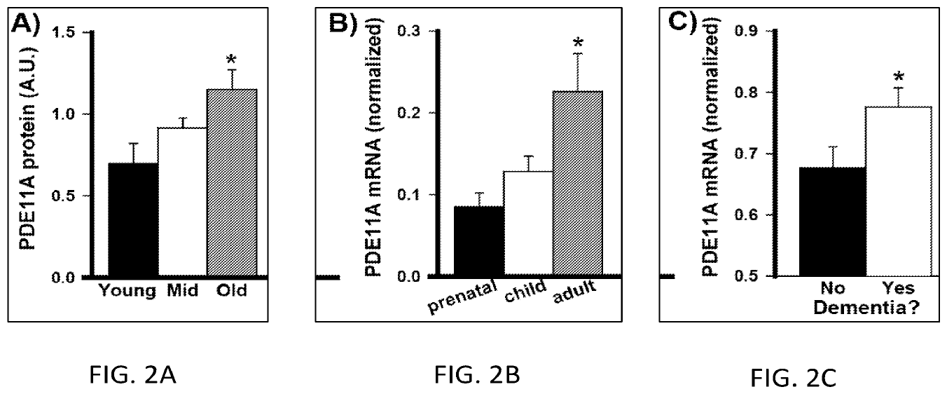

While not being bound by any particular theory, it is hypothesized that cAMP and cGMP signaling are decreased in the aged and demented hippocampus (rodents and humans), particularly when there is a history of traumatic brain injury (TBI). These age-related decreases in cyclic nucleotides are consistent with the observations that PDE11A4 expression increases with age in the rodent and human hippocampus and is significantly elevated in hippocampus of demented vs. non-demented aged humans with a history of TBI (FIG. 2).

Consistent with PDE11A4's restricted expression pattern, PDE11A KO mice appear normal on a wide range of sensory, motor and anxiety/depression-related behaviors, and show no gross peripheral pathology at least up to 1 year of age (later ages not assessed). Instead, PDE11A KO mice exhibit select social phenotypes such as preferring to interact with other PDE11A KO mice vs wild-type (WT) mice and showing differences in the consolidation of social memories. PDE11A KO mice also have an increased sensitivity to the behavioral effects of lithium. Little has been characterized of the signaling pathways lying up or downstream of PDE11A4, but it was shown that PDE11A appears to regulate signals that are important for memory consolidation, including glutamatergic and calcium/calmodulin-dependent kinase II (CamKII) signaling as well as protein synthesis.

The longest isoform, PDE11A4, is ~95% homologous across mouse, rat and human. Tissue-specific distribution and function of other isoforms is discussed by others, and these isoforms are not present in the CNS. Given the paucity of PDE11A inhibitor discovery to date, no information on inhibition of these isoforms is in the public domain. In the brain, PDE11A4 is strongly expressed in neurons of the ventral hippocampal formation (VHIPP; a.k.a. anterior hippocampus in primates), with much lower levels of expression in dorsal hippocampus as well as the adjacent amygdalohippocampal region and in some mice the nearby amygdalostriatal transition area. Outside of the brain, PDE11A4 expression was reliably measured in the spinal cord and dorsal root ganglion (i.e., present in wild-type but not Pde11a knockout mice), with no reliable PDE11A4 expression observed in 20 peripheral organs. This makes PDE11A4 unique because in brain it is the only PDE to be expressed preferentially in the VHIPP, a structure critical for associative long-term memories. This along with the fact that PDE11A is a druggable enzyme makes PDE11A an attractive drug target because it stands to selectively restore aberrant cyclic nucleotide signaling in a brain region affected by various disease states without directly affecting signaling in other brain regions or peripheral organs. Indeed, Pde11a KO mice appear normal on a wide range of sensory, motor and anxiety/depression-related behaviors, show no gross peripheral histopathology at least up to 1 year of age (later ages not assessed), and reproduce normally.

Interestingly, PDE11A4 expression in the hippocampus increases across the lifespan of both humans and rodents. This age-related increase in PDE11A4 is consistent with literature showing decreases in cAMP and cGMP in the aged and demented hippocampus (rodents and humans), particularly when there is a history of traumatic brain injury (TBI). In vitro and rodent studies have shown that age-related increases in PDE11A4 expression are driven by increased phosphorylation of the N-terminal regulatory domain at S117 and S124. Rodent studies also have shown these age-related increases in PDE11A4 expression drive age-related cognitive decline of social associative memories due to increased presence of the protein in the aged brain as opposed to a prolonged effect on the development of the brain. This is consistent with the fact that PDE11A4 regulates signals important for memory consolidation, including glutamatergic and calcium/calmodulin-dependent kinase II (CamKII) signaling as well as protein synthesis. While not wishing to be bound by any particular theory, these results suggest that a PDE11A4 inhibitor can be useful for reversing some aspects of age-related cognitive decline.

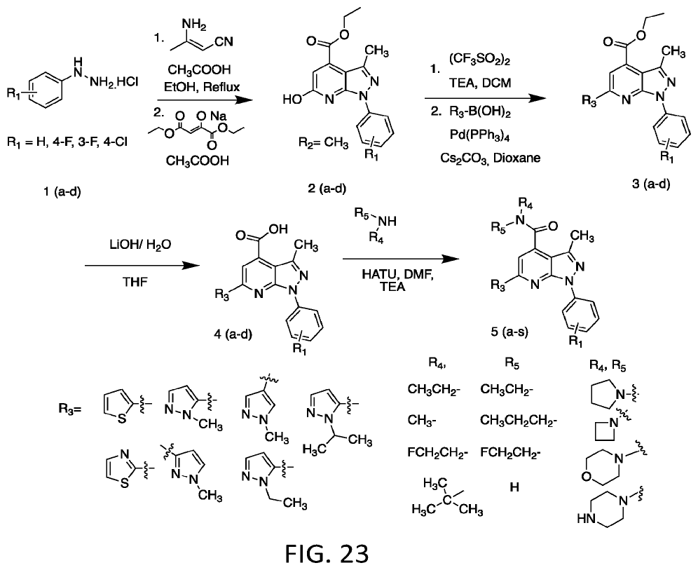

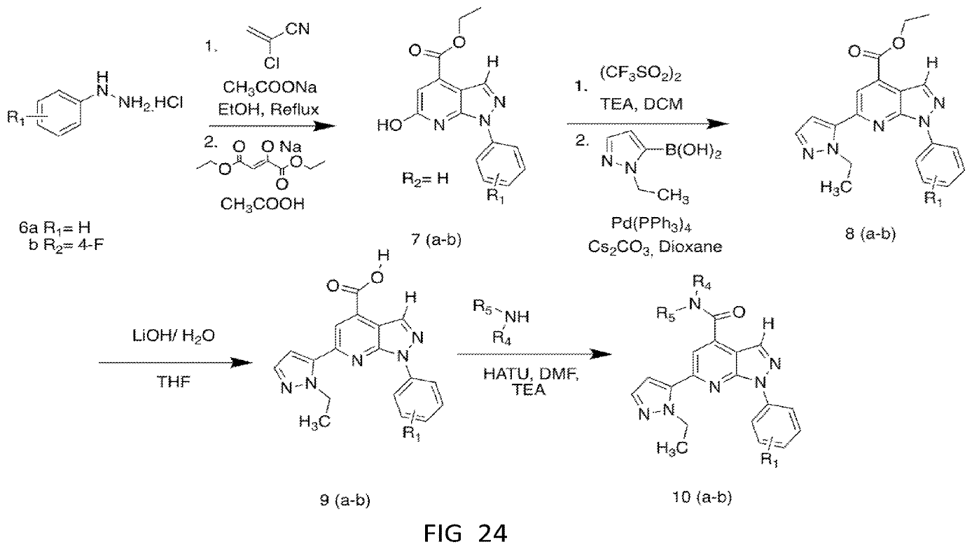

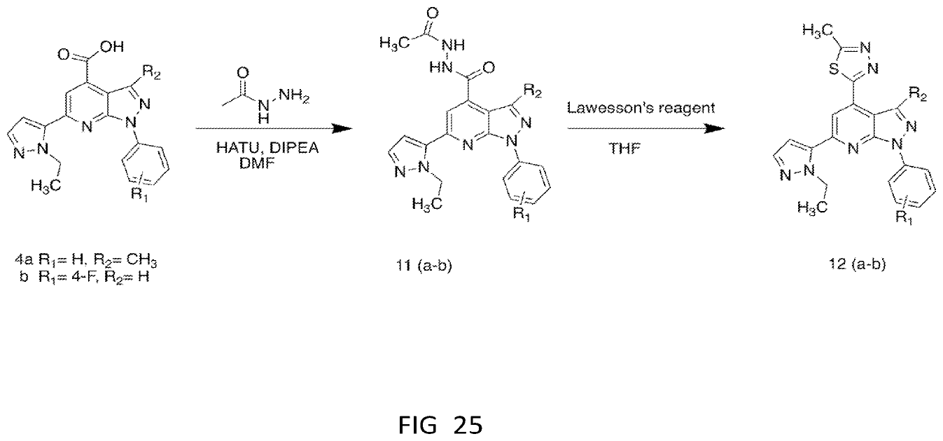

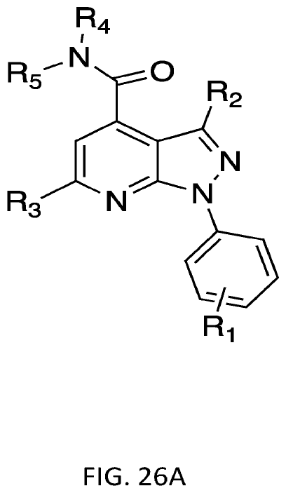

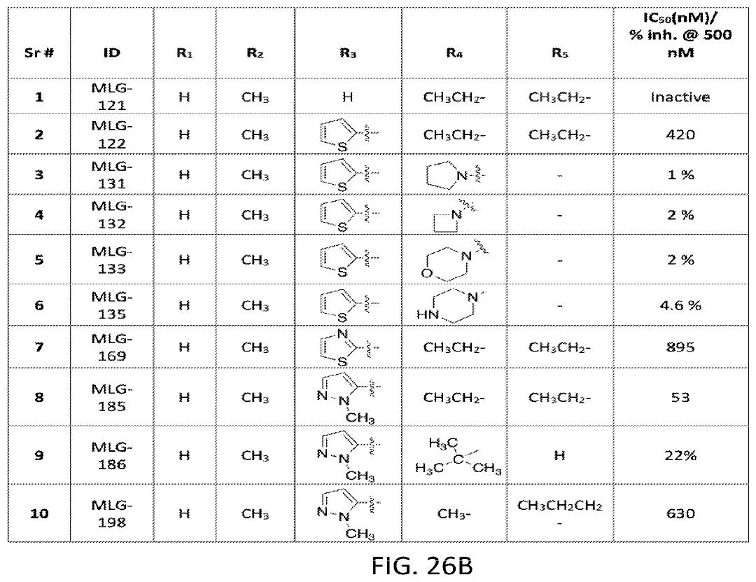

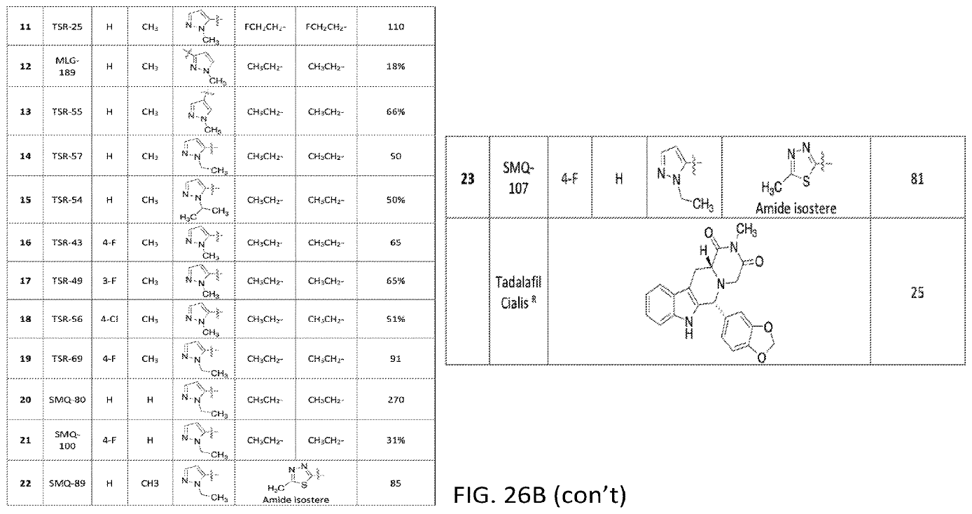

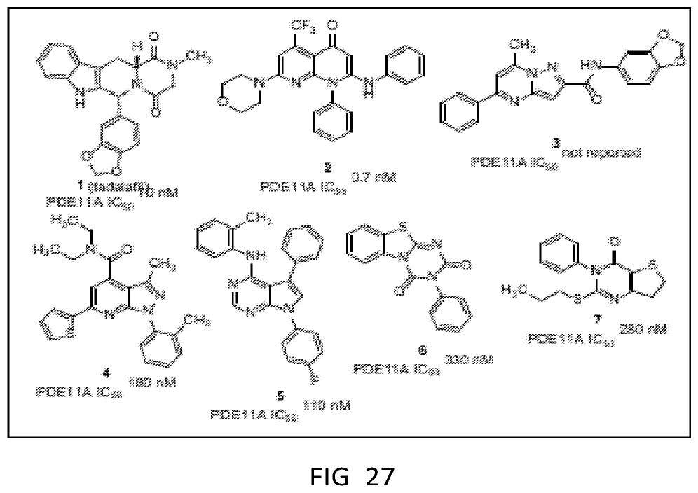

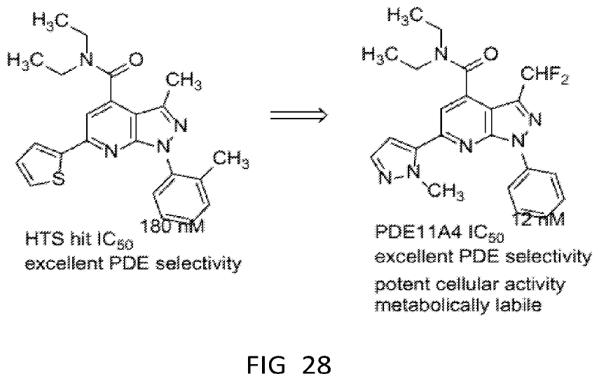

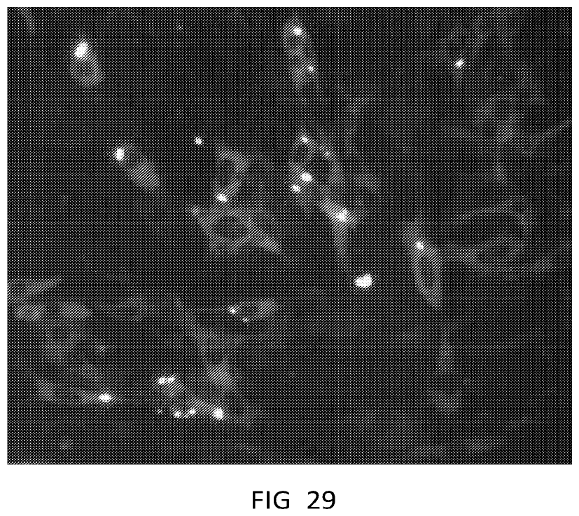

FIG. 23 is an image showing a non-limiting example of a scheme of a synthesis of compounds 5 (a-s). FIG. 24 is an image showing a non-limiting example of a scheme to prepare compounds 10 (a-b). FIG. 25 is an image showing a non-limiting example of a scheme to prepare compounds 12 (a-b). FIG. 26A is an image of exemplary PDE11A inhibitors of the disclosure. FIG. 26B is an image and a table. FIG. 27 is an image of exemplary PDE11A inhibitors of the disclosure. FIG. 28 is an image of exemplary PDE11A inhibitors of the disclosure. FIG. 29 is an image of HT22 cells overexpressing EmGFP-mPDE11A4.

Briefly, PDEs are discretely localized to specific subcellular domains. As a result, PDEs do not simply control the total cellular content of cyclic nucleotides, they generate individual pools or nanodomains of cyclic nucleotide signaling. Such subcellular compartmentalization of cyclic nucleotide signaling allows a single cell to respond discretely to diverse intra- and extracellular signals. Thus, where a PDE is localized is just as important to its overall function as is its catalytic activity. While some cyclases and PDEs responsible for generating and breaking down cAMP/cGMP are expressed more in the cytosol than the membrane (like PDE11A4), others are enriched in the membrane (like the closely related PDE2A and PDE10A). Interestingly, cyclic nucleotide signaling deficits observed in bipolar disorder and Alzheimer's disease appear to be more prominent in the cytosolic as opposed to membrane fractions.

Associative long-term memories (aLTMs)—particularly those involving friends and family—are more susceptible to age-related cognitive decline than are recognition long-term memories (rLTMs) for reasons that are not well understood. The lack of knowledge of the molecular mechanisms that govern age-related decline slows the development of novel therapeutics. Age-related increases in phosphodiesterase 11A (PDE11A), an enzyme that breaks down cAMP/cGMP and regulates social behaviors, may be a fundamental mechanism underlying age-related cognitive decline of aLTMs for social experiences. With regard to its tissue expression profile, the best controlled studies to date suggest that the longest isoform PDE11A4 is almost exclusively expressed in the ventral hippocampal formation (a.k.a. anterior HIPP in primates), specifically within neurons of the subiculum, superficial layer of CA1, and the adjacently connected amygdalohippocampal area. This makes PDE11A4 the ONLY PDE to be preferentially expressed in the HIPP, a brain region key to social aLTMs. Previous studies suggest that cAMP and cGMP signaling are decreased in the aging and demented HIPP (rodents and humans), consistent with the novel observations of aging and dementia-related increases in HIPP PDE11A4 expression in rodents and humans. Decreases in 3′,5′-cyclic nucleotide signaling in the aged and demented hippocampus contribute to cognitive decline and are related, in part, to changes in the enzymes that break down cyclic nucleotides. There are eleven families of 3′,5′-cyclic nucleotide phosphodiesterases (PDEs) that are the only known enzymes to hydrolyze 3′,5′-cyclic adenosine monophosphate (cAMP) and 3′,5′-cyclic guanosine monophosphoate (cGMP), and their expression across subcellular compartments differs between families. For example, some PDEs are expressed more in the cytosol than the membrane (e.g., PDE1 TA), while others are more highly expressed in the membrane versus cytosol (including PDE2A, PDE9A and PDE10A. Due to the fact that PDEs are localized to specific subcellular domains, they are able to regulate individual pools or nanodomains of cyclic nucleotide signaling. Such subcellular compartmentalization of cyclic nucleotide signaling allows a single cell to respond specifically to simultaneous intra- and/or extracellular signals. Therefore, the subcellular localization of any PDE is equally important to its actual catalytic activity when considering its function. PDEs can become overexpressed and/or mislocalized with age and/or disease, which compromises the integrity of this physiological segregation of signals. Indeed, age-related diseases and neuropsychiatric diseases can show a loss of cyclic nucleotide signaling in one subcellular compartment but not another, suggesting therapeutic strategies should optimally target enzymes in a compartment-specific manner.

Of the eleven families of PDEs, phosphodiesterase 11A (PDE11A) has garnered particular interest in the context of altered cyclic nucleotide signaling related to ARCD and early-onset Alzheimer's disease. PDE11A is encoded by a single gene and has four isoforms. While protein for PDE11A4—the isoform expressed in brain—is found across all subcellular compartments, it is particularly enriched in the cytosolic versus membrane and nuclear compartments. The PDE11A catalytic domain is located within the C-terminal region, which is common to all isoforms, while the N-terminal region serves a regulatory function and is unique to each isoform. The regulatory N-terminus of PDE11A4, the longest PDE11A isoform, is unique in that it contains two full GAF (cGMP binding PDE, Anabaena adenylyl cyclase and E. coli FhlA) domains. The GAF-A domain binds cGMP as a potential allosteric regulatory site and the GAF-B domain regulates protein-protein interactions, including homodimerization. PDE11A4 is unique in that it is the only PDE whose expression in brain emanates solely from the extended hippocampal formation, a brain region critical to learning and memory and vulnerable to age-related deficits in cyclic nucleotide signaling. Possibly contributing to these hippocampal cyclic nucleotide signaling deficits are age-related increases in PDE11A4 expression that are conserved across mice, rats and humans. These age-related increases in PDE11A4 protein expression are deleterious as 1) PDE11A KO mice are protected against age-related cognitive decline (ARCD) of remote social associative long-term memories (aLTMs) and 2) mimicking age-related overexpression of PDE11A4 in the CAT field of hippocampus of either young or old PDE11A KO mice is sufficient to mimic ARCD of remote social aLTMs.

While PDE11A4 protein expression in the young adult hippocampus is significantly higher in the cytosolic versus membrane compartment, age-related increases in PDE11A4 are found specifically in the membrane compartment of the ventral hippocampal formation (VHIPP). Therefore, these age-related increases in membrane-associated PDE11A4 may be considered ectopic. That said, the membrane pool of PDE11A4—although lesser in relative quantity-continually shows itself to be critical in regulating social behaviors. For instance, it was found that social isolation selectively decreases expression of membrane-associated PDE11A4 in the VHIPP, and that these isolation-induced decreases in PDE11A4 are sufficient to cause changes in subsequent social preferences and social memory. Similarly, PDE11A4 protein expression differences in VHIPP of BALB/cJ versus C₅₇BL/6J mice are restricted to the membrane pool. The increased protein expression of membrane-associated PDE11A4 found in the VHIPP of BALB/cJ vs C₅₇BL/6J mice appears to be driven by a single point mutation at amino acid 499 within the GAF-B domain, which strengthens the homodimerization and punctate accumulation of PDE11A4. Interestingly, disrupting PDE11A4 homodimerization in vitro by expressing an isolated GAF-B domain that acts as a negative sink disperses the punctate accumulation of PDE11A4 and selectively reduces expression of membrane-associated PDE11A4. This suggests that disrupting PDE11A4 homodimerization in vivo may represent a therapeutic option capable of treating age-related increases in PDE11A4 expression in a compartment-specific manner and, thus, ARCD of social memories. Indeed, it was previously showed that social preference of C₅₇BL/6J mice can be altered by manipulating PDE11A4 homodimerization selectively within the CAT field of hippocampus.

In one aspect, the disclosure provides a novel class of PDE11A4 modulators, which is a useful therapeutic target for treating social deficits associated with schizophrenia, bipolar disorder, or autism as well as for treating cognitive deficits/dementia associated with age-related cognitive decline, traumatic brain injury, or Alzheimer's disease.

In one aspect, the disclosure provides compounds that inhibit the activity of PDE11A4 and are capable of 1) altering social preferences/compatibility within the context of neuropsychiatric or neurodevelopmental disorders, 2) reversing cognitive decline associated with aging, dementia associated with traumatic brain injury, and/or Alzheimer's disease, and/or 3) alleviating other disorders where PDE11A4 forms accumulated proteinopathies, particularly in the membrane fraction. In a non-limiting embodiments, the role of PDE11A4 in actual brain function is explored, and intramolecular mechanisms that control how PDE11A4 functions in terms of enzymatic activity and subcellular trafficking are examined.

### PDE11A4 Inhibitors and Methods of Inhibiting PDE11A4

In an embodiment, the disclosure includes molecules capable of inhibiting PDE11A. In one embodiment, the compounds described herein are PDE11A inhibitors capable of inhibiting PDE11A1, PDE11A2, PDE11A3, and/or PDE11A4. In one embodiment, the PDE11A inhibitor is a PDE11A1 inhibitor. In one embodiment, the PDE11A inhibitor is a PDE11A2 inhibitor. In one embodiment, the PDE11A inhibitor is a PDE11A3 inhibitor. In one embodiment, the PDE11A inhibitor is a PDE11A4 inhibitor. In one embodiment, the PDE1 TA4 inhibitor is a PDE11A4 selective inhibitor. In an embodiment, the PDE11A4 inhibitor of the disclosure is about 1-fold, about 5-fold, about 10-fold, about 20-fold, about 50-fold, about 100-fold, about 500-fold, or about 1000-fold more selective for PDE11A4 over PDE11A1, PDE11A2, and/or PDE11A3.

In one aspect, the disclosure provides a compound of formula (A), or a pharmaceutically acceptable salt, solvate, hydrate, cocrystal, or prodrug thereof:

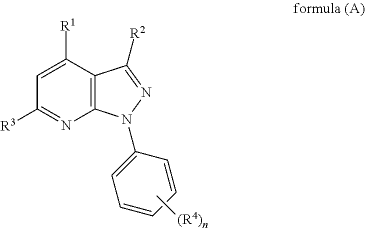

wherein in formula (A):
R¹ is selected from 5-membered heterocycle, 5-membered heteroaryl, 6-membered heterocycle, and 6-membered heteroaryl; or R¹ is

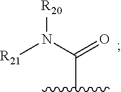

R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
R₂₀ is selected from H, optionally substituted alkyl, and optionally substituted haloalkyl;
R₂₁ is selected from optionally substituted alkyl and optionally substituted haloalkyl; or R₂₀ and R₂₁ are joined to form a 4-7 membered ring with the nitrogen to which they are each bound, wherein the ring may comprise 1-3 additional heteroatoms selected from O, N, NH, and S;
n is an integer selected from 0-5; and
t is 1 or 2.

In one aspect, the disclosure provides a compound of formula (I), or a pharmaceutically acceptable salt, solvate, hydrate, cocrystal, or prodrug thereof:

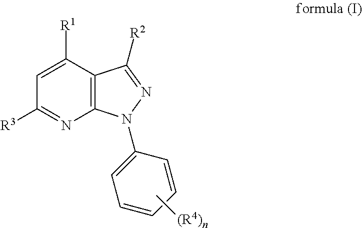

wherein in formula (I):
R¹ and R³ are independently at each occurrence a 5-membered heterocycle, 5-membered heteroaryl, a 6-membered heterocycle, or 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

In some embodiments, R¹ is a 5-membered heteroaryl ring. In some embodiments, R¹ is selected from thiazole, oxazole, imidazole, thiadiazole, oxadiazole, and triazole.

In some embodiments, the compound of formula (A) or formula (I) is a compound having formula (II), or a pharmaceutically acceptable salt, solvate, hydrate, cocrystal, or prodrug thereof:

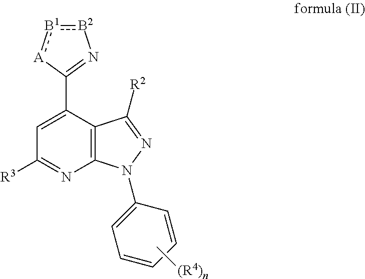

wherein in formula (II):
A is S, NR⁵, N, or O, optionally A is S or O;
B¹ and B² are independently at each occurrence selected from N, O, or CR⁵, wherein B¹ is not O when either A or B² is O; and
each R⁵ is independently at each occurrence selected from hydrogen, C₁-C₆ alkyl (e.g., C₁-C₃ alkyl), and C₁-C₃ haloalkyl (e.g., CH₂F, CHF₂, CF₃);
R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R)₂, —C(O)R^{a}, —C(O)OR^{a}, —C(O)SR^{a}, —OC(O)N(R^{a})₂, —C(O)N(R)₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
n is an integer selected from 0-5; and
t is 1 or 2.

In some embodiments, B¹ and B² cannot both be O simultaneously. In some embodiments, A and B¹ cannot both be O simultaneously. In some embodiments, B¹ and B² cannot both be O simultaneously, and A and B¹ cannot both be O simultaneously.

In some embodiments, R¹ is selected from

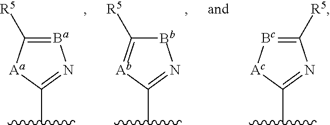

wherein: A^{a} is O or S, and B^{a} is N or CH; A^{b} is N, and B^{b} is O or S; A^{c} is O or S, and B^{c} is N; and R⁵ is selected from hydrogen, C₁-C₃ alkyl (e.g., methyl, ethyl, propyl, isopropyl), and C₁-C₃ haloalkyl (e.g., CH₂F, CHF₂, or CF₃).

In some embodiments, R¹ is selected from

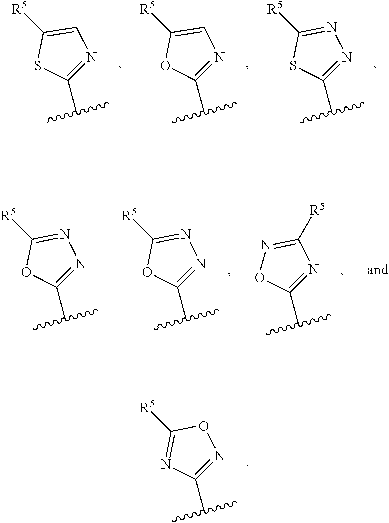

In some embodiments, R⁵ is selected from H, methyl, ethyl, propyl, isopropyl, CH₂F, and CHF₂. In some embodiments, R⁵ is C₁-C₃ alkyl.

In some embodiments, R¹ is selected from

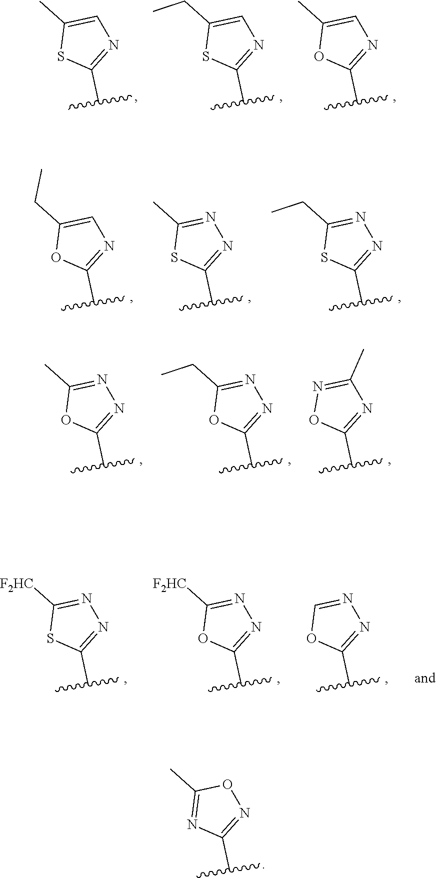

In some embodiments, R₂ is selected from hydrogen, C₃-C₆ cycloalkyl, C₁-C₆ alkyl (e.g., C₁-C₄ straight chain alkyl, C₃-C₆ branched alkyl), —CF₃, —CH₂F, —CHF₂, and —C(R⁶R⁷)ₚX; wherein, p is an integer from 1-3; R⁶ and R⁷ are each independently selected from hydrogen, C₁-C₃ alkyl and C₃-C₆ cycloalkyl; X is selected from —OH, —CN, —OR⁸, and NR⁹R¹⁰; R⁸ is selected from C₁-C₃ alkyl, and C₃-C₆ cycloalkyl; and R⁹ and R¹⁰ are each independently selected from hydrogen, C₁-C₃ alkyl and C₃-C₆ cycloalkyl. In some embodiments, R₂ is C₁-C₄ straight chain alkyl. In some embodiments, R⁸ is C₁-C₃ straight or branched alkyl.

In some embodiments, R₂ is selected from hydrogen and C₁-C₆ alkyl, optionally —CH₃.

In some embodiments, R₂ is selected from hydrogen, C₁-C₄ alkyl (e.g., methyl, ethyl, propyl, isopropyl, butyl, tertbutyl), —CF₃, —CH₂F, and —CHF₂. In some embodiments, R₂ is selected from H, —CH₃, —CF₃, and —CHF₂.

In some embodiments, R³ is a 5-membered heteroaryl ring. In some embodiments, R³ is selected from pyrrole, thiophene, furan, pyrazole, thiazole, isothiazole, oxazole, isoxazole, and imidazole.

In some embodiments, R³ is selected from

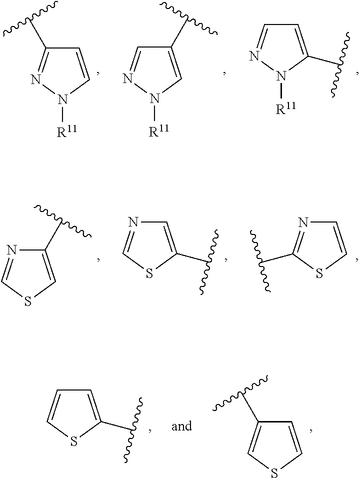

wherein R¹¹ is selected from hydrogen, C₁-C₃ alkyl (e.g., methyl, ethyl, propyl, isopropyl), C₃-C₆ cycloalkyl, CF₃, CH₂CF₃, CH₂CHF₂, and CH₂CH₂F. In some embodiments, R³ is C₁-C₃ straight or branched alkyl.

In some embodiments, R³ is selected from

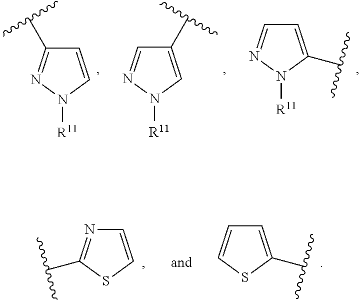

In some embodiments, R¹¹ is selected from hydrogen, methyl, ethyl, propyl, isopropyl, —CF₃, and —CH₂CF₃.

In some embodiments R³ is selected from

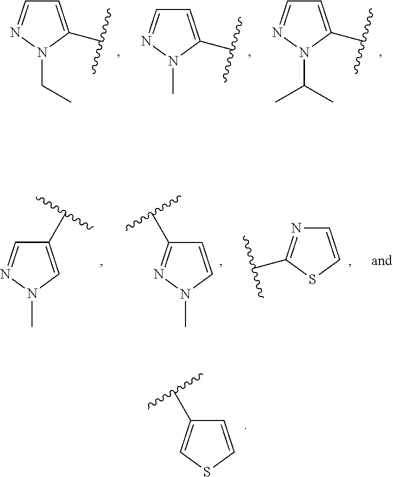

In some embodiments, R³ is selected from

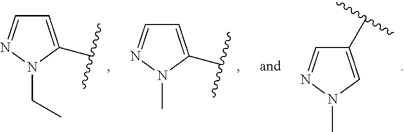

In some embodiments, R⁴ is selected from hydrogen, C₁-C₆ alkyl, and halo (e.g., F or Cl). In some embodiments, R⁴ is F or C₁. In some embodiments, n is 0, 1, or 2. In some embodiments, n is 0 or 1.

In some embodiments, R⁴ is selected from hydrogen, 4-fluoro, 3-fluoro, 2-fluoro, 2-chloro, and 4-chloro. In some embodiments, R⁴ is selected from hydrogen, 4-fluoro, 2-fluoro, and 2-chloro. In some embodiments, n is 2 and each R⁴ is 2-fluoro and 4-fluoro.

In some embodiments, the compound of formula (A), formula (I), or formula (II) is a compound having any one of formula 1001-1729 or 4001-4729, or a pharmaceutically acceptable salt, solvate, hydrate, cocrystal, or prodrug thereof.

Formula (Ia)

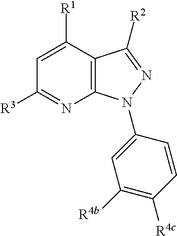

formula (Ib)

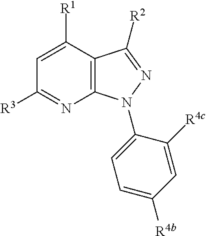

| Cpd. # | Cpd. # | | | | | |
|---|---|---|---|---|---|---|
| Formula (Ia) | Formula (Ib) | R¹ | R² | R³ | R^{4b} | R^{4c} |
| 1001 | 4001 | 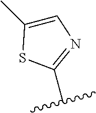 [SMILES: *c1ncc(C)s1] | H | 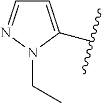 [SMILES: *c1ccnn1CC] | H | H |
| 1002 | 4002 | 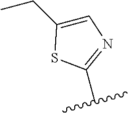 [SMILES: *c1ncc(CC)s1] | H | 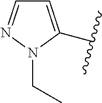 [SMILES: *c1ccnn1CC] | H | H |
| 1003 | 4003 | 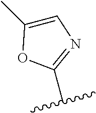 [SMILES: *c1ncc(C)o1] | H | 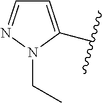 [SMILES: *c1ccnn1CC] | H | H |
| 1004 | 4004 | 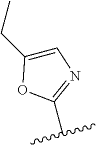 [SMILES: *c1ncc(CC)o1] | H | 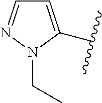 [SMILES: *c1ccnn1CC] | H | H |
| 1005 | 4005 | 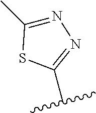 [SMILES: *c1nnc(C)s1] | H | 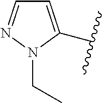 [SMILES: *c1ccnn1CC] | H | H |
| 1006 | 4006 | 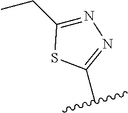 [SMILES: *c1nnc(CC)s1] | H | 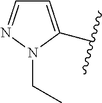 [SMILES: *c1ccnn1CC] | H | H |
| 1007 | 4007 | 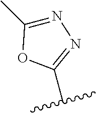 [SMILES: *c1nnc(C)o1] | H | 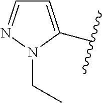 [SMILES: *c1ccnn1CC] | H | H |
| 1008 | 4008 | 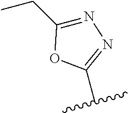 [SMILES: *c1nnc(CC)o1] | H | 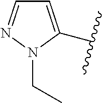 [SMILES: *c1ccnn1CC] | H | H |
| 1009 | 4009 | 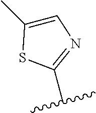 [SMILES: *c1ncc(C)s1] | CH₃ | 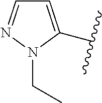 [SMILES: *c1ccnn1CC] | H | H |
| 1010 | 4010 | 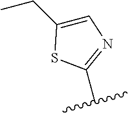 [SMILES: *c1ncc(CC)s1] | CH₃ | 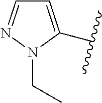 [SMILES: *c1ccnn1CC] | H | H |
| 1011 | 4011 | 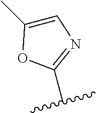 [SMILES: *c1ncc(C)o1] | CH₃ | 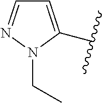 [SMILES: *c1ccnn1CC] | H | H |
| 1012 | 4012 | 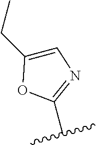 [SMILES: *c1ncc(CC)o1] | CH₃ | 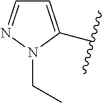 [SMILES: *c1ccnn1CC] | H | H |
| 1013 | 4013 | 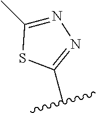 [SMILES: *c1nnc(C)s1] | CH₃ | 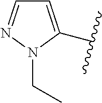 [SMILES: *c1ccnn1CC] | H | H |
| 1014 | 4014 | 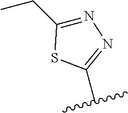 [SMILES: *c1nnc(CC)s1] | CH₃ | 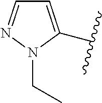 [SMILES: *c1ccnn1CC] | H | H |
| 1015 | 4015 | 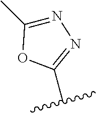 [SMILES: *c1nnc(C)o1] | CH₃ | 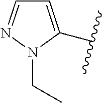 [SMILES: *c1ccnn1CC] | H | H |
| 1016 | 4016 | 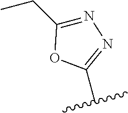 [SMILES: *c1nnc(CC)o1] | CH₃ | 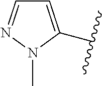 [SMILES: *c1ccnn1C] | H | H |
| 1017 | 4017 | 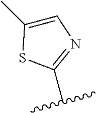 [SMILES: *c1ncc(C)s1] | H | 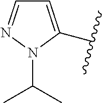 [SMILES: *c1ccnn1C(C)C] | H | H |
| 1018 | 4018 | 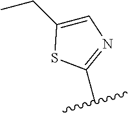 [SMILES: *c1ncc(CC)s1] | H | 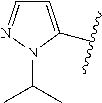 [SMILES: *c1ccnn1C(C)C] | H | H |
| 1019 | 4019 | 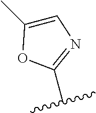 [SMILES: *c1ncc(C)o1] | H | 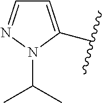 [SMILES: *c1ccnn1C(C)C] | H | H |
| 1020 | 4020 | 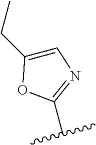 [SMILES: *c1ncc(CC)o1] | H | 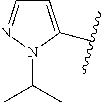 [SMILES: *c1ccnn1C(C)C] | H | H |
| 1021 | 4021 | 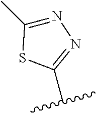 [SMILES: *c1nnc(C)s1] | H | 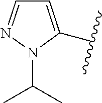 [SMILES: *c1ccnn1C(C)C] | H | H |
| 1022 | 4022 | 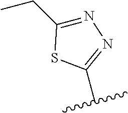 [SMILES: *c1nnc(CC)s1] | H | 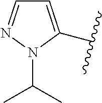 [SMILES: *c1ccnn1C(C)C] | H | H |
| 1023 | 4023 | 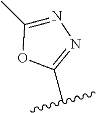 [SMILES: *c1nnc(C)o1] | H | 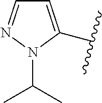 [SMILES: *c1ccnn1C(C)C] | H | H |
| 1024 | 4024 | 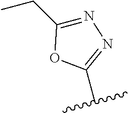 [SMILES: *c1nnc(CC)o1] | H | 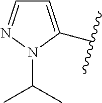 [SMILES: *c1ccnn1C(C)C] | H | H |
| 1025 | 4025 | 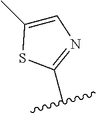 [SMILES: *c1ncc(C)s1] | CH₃ | 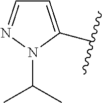 [SMILES: *c1ccnn1C(C)C] | H | H |
| 1026 | 4026 | 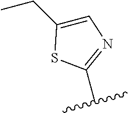 [SMILES: *c1ncc(CC)s1] | CH₃ | 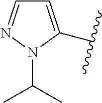 [SMILES: *c1ccnn1C(C)C] | H | H |
| 1027 | 4027 | 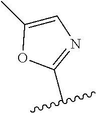 [SMILES: *c1ncc(C)o1] | CH₃ | 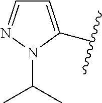 [SMILES: *c1ccnn1C(C)C] | H | H |
| 1028 | 4028 | 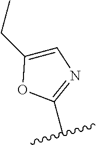 [SMILES: *c1ncc(CC)o1] | CH₃ | 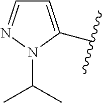 [SMILES: *c1ccnn1C(C)C] | H | H |
| 1029 | 4029 | 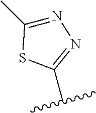 [SMILES: *c1nnc(C)s1] | CH₃ | 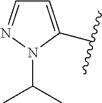 [SMILES: *c1ccnn1C(C)C] | H | H |
| 1030 | 4030 | 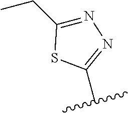 [SMILES: *c1nnc(CC)s1] | CH₃ | 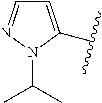 [SMILES: *c1ccnn1C(C)C] | H | H |
| 1031 | 4031 | 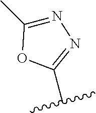 [SMILES: *c1nnc(C)o1] | CH₃ | 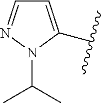 [SMILES: *c1ccnn1C(C)C] | H | H |
| 1032 | 4032 | 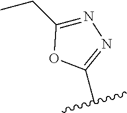 [SMILES: *c1nnc(CC)o1] | CH₃ | 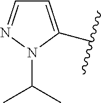 [SMILES: *c1ccnn1C(C)C] | H | H |
| 1033 | 4033 | 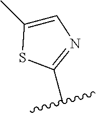 [SMILES: *c1ncc(C)s1] | H | 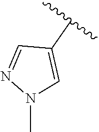 [SMILES: *c1cnn(C)c1] | H | H |
| 1034 | 4034 | 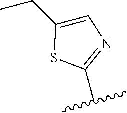 [SMILES: *c1ncc(CC)s1] | H | 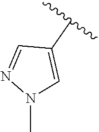 [SMILES: *c1cnn(C)c1] | H | H |
| 1035 | 4035 | 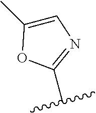 [SMILES: *c1ncc(C)o1] | H | 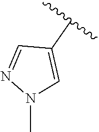 [SMILES: *c1cnn(C)c1] | H | H |
| 1036 | 4036 | 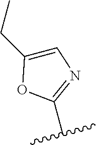 [SMILES: *c1ncc(CC)o1] | H | 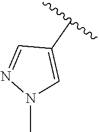 [SMILES: *c1cnn(C)c1] | H | H |
| 1037 | 4037 | 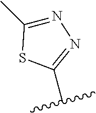 [SMILES: *c1nnc(C)s1] | H | 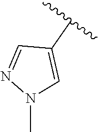 [SMILES: *c1cnn(C)c1] | H | H |
| 1038 | 4038 | 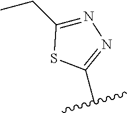 [SMILES: *c1nnc(CC)s1] | H | 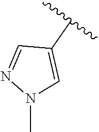 [SMILES: *c1cnn(C)c1] | H | H |
| 1039 | 4039 | 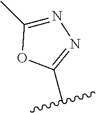 [SMILES: *c1nnc(C)o1] | H | 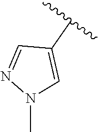 [SMILES: *c1cnn(C)c1] | H | H |
| 1040 | 4040 | 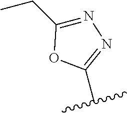 [SMILES: *c1nnc(CC)o1] | H | 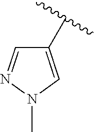 [SMILES: *c1cnn(C)c1] | H | H |
| 1041 | 4041 | 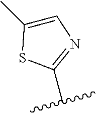 [SMILES: *c1ncc(C)s1] | CH₃ | 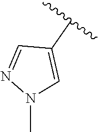 [SMILES: *c1cnn(C)c1] | H | H |
| 1042 | 4042 | 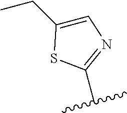 [SMILES: *c1ncc(CC)s1] | CH₃ | 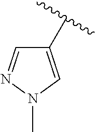 [SMILES: *c1cnn(C)c1] | H | H |
| 1043 | 4043 | 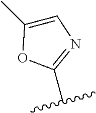 [SMILES: *c1ncc(C)o1] | CH₃ | 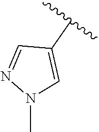 [SMILES: *c1cnn(C)c1] | H | H |
| 1044 | 4044 | 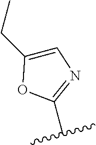 [SMILES: *c1ncc(CC)o1] | CH₃ | 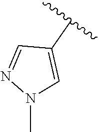 [SMILES: *c1cnn(C)c1] | H | H |
| 1045 | 4045 | 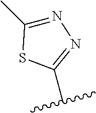 [SMILES: *c1nnc(C)s1] | CH₃ | 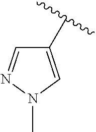 [SMILES: *c1cnn(C)c1] | H | H |
| 1046 | 4046 | 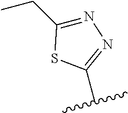 [SMILES: *c1nnc(CC)s1] | CH₃ | 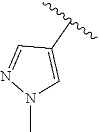 [SMILES: *c1cnn(C)c1] | H | H |
| 1047 | 4047 | 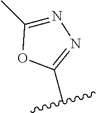 [SMILES: *c1nnc(C)o1] | CH₃ | 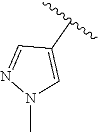 [SMILES: *c1cnn(C)c1] | H | H |
| 1048 | 4048 | 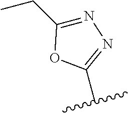 [SMILES: *c1nnc(CC)o1] | CH₃ | 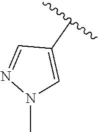 [SMILES: *c1cnn(C)c1] | H | H |
| 1049 | 4049 | 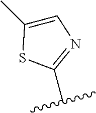 [SMILES: *c1ncc(C)s1] | H | 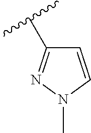 [SMILES: *c1ccn(C)n1] | H | H |
| 1050 | 4050 | 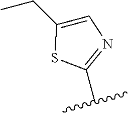 [SMILES: *c1ncc(CC)s1] | H | 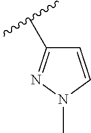 [SMILES: *c1ccn(C)n1] | H | H |
| 1051 | 4051 | 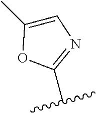 [SMILES: *c1ncc(C)o1] | H | 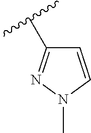 [SMILES: *c1ccn(C)n1] | H | H |
| 1052 | 4052 | 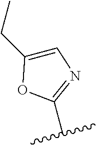 [SMILES: *c1ncc(CC)o1] | H | 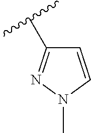 [SMILES: *c1ccn(C)n1] | H | H |
| 1053 | 4053 | 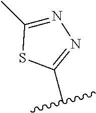 [SMILES: *c1nnc(C)s1] | H | 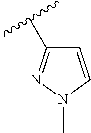 [SMILES: *c1ccn(C)n1] | H | H |
| 1054 | 4054 | 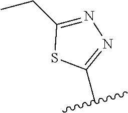 [SMILES: *c1nnc(CC)s1] | H | 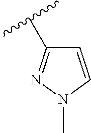 [SMILES: *c1ccn(C)n1] | H | H |
| 1055 | 4055 | 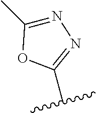 [SMILES: *c1nnc(C)o1] | H | 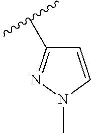 [SMILES: *c1ccn(C)n1] | H | H |
| 1056 | 4056 | 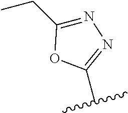 [SMILES: *c1nnc(CC)o1] | H | 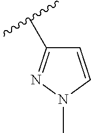 [SMILES: *c1ccn(C)n1] | H | H |
| 1057 | 4057 | 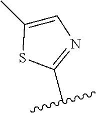 [SMILES: *c1ncc(C)s1] | CH₃ | 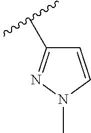 [SMILES: *c1ccn(C)n1] | H | H |
| 1058 | 4058 | 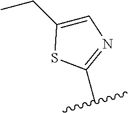 [SMILES: *c1ncc(CC)s1] | CH₃ | 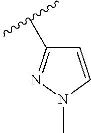 [SMILES: *c1ccn(C)n1] | H | H |
| 1059 | 4059 | 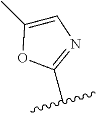 [SMILES: *c1ncc(C)o1] | CH₃ | 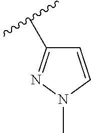 [SMILES: *c1ccn(C)n1] | H | H |
| 1060 | 4060 | 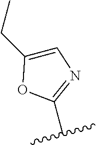 [SMILES: *c1ncc(CC)o1] | CH₃ | 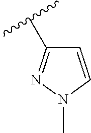 [SMILES: *c1ccn(C)n1] | H | H |
| 1061 | 4061 | 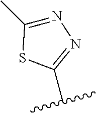 [SMILES: *c1nnc(C)s1] | CH₃ | 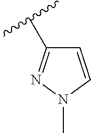 [SMILES: *c1ccn(C)n1] | H | H |
| 1062 | 4062 | 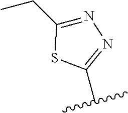 [SMILES: *c1nnc(CC)s1] | CH₃ | 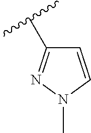 [SMILES: *c1ccn(C)n1] | H | H |
| 1063 | 4063 | 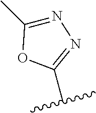 [SMILES: *c1nnc(C)o1] | CH₃ | 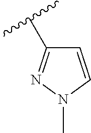 [SMILES: *c1ccn(C)n1] | H | H |
| 1064 | 4064 | 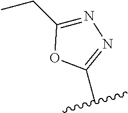 [SMILES: *c1nnc(CC)o1] | CH₃ | 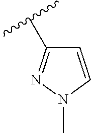 [SMILES: *c1ccn(C)n1] | H | H |
| 1065 | 4065 | 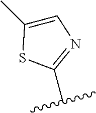 [SMILES: *c1ncc(C)s1] | H | 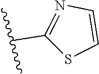 [SMILES: *c1nccs1] | H | H |
| 1066 | 4066 | 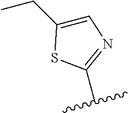 [SMILES: *c1ncc(CC)s1] | H | 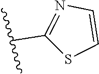 [SMILES: *c1nccs1] | H | H |
| 1067 | 4067 | 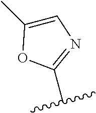 [SMILES: *c1ncc(C)o1] | H | 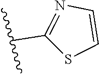 [SMILES: *c1nccs1] | H | H |
| 1068 | 4068 | 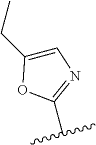 [SMILES: *c1ncc(CC)o1] | H | 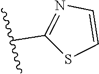 [SMILES: *c1nccs1] | H | H |
| 1069 | 4069 | 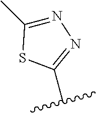 [SMILES: *c1nnc(C)s1] | H | 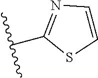 [SMILES: *c1nccs1] | H | H |
| 1070 | 4070 | 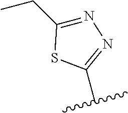 [SMILES: *c1nnc(CC)s1] | H | 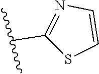 [SMILES: *c1nccs1] | H | H |
| 1071 | 4071 | 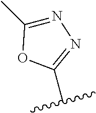 [SMILES: *c1nnc(C)o1] | H | 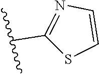 [SMILES: *c1nccs1] | H | H |
| 1072 | 4072 | 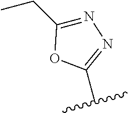 [SMILES: *c1nnc(CC)o1] | H | 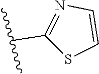 [SMILES: *c1nccs1] | H | H |
| 1073 | 4073 | 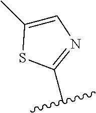 [SMILES: *c1ncc(C)s1] | CH₃ | 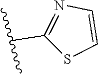 [SMILES: *c1nccs1] | H | H |
| 1074 | 4074 | 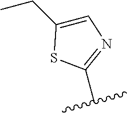 [SMILES: *c1ncc(CC)s1] | CH₃ | 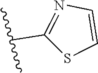 [SMILES: *c1nccs1] | H | H |
| 1075 | 4075 | 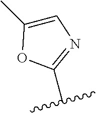 [SMILES: *c1ncc(C)o1] | CH₃ | 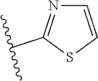 [SMILES: *c1nccs1] | H | H |
| 1076 | 4076 | 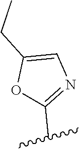 [SMILES: *c1ncc(CC)o1] | CH₃ | 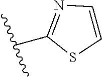 [SMILES: *c1nccs1] | H | H |
| 1077 | 4077 | 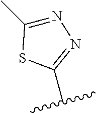 [SMILES: *c1nnc(C)s1] | CH₃ | 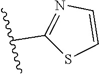 [SMILES: *c1nccs1] | H | H |
| 1078 | 4078 | 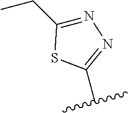 [SMILES: *c1nnc(CC)s1] | CH₃ | 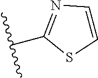 [SMILES: *c1nccs1] | H | H |
| 1079 | 4079 | 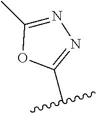 [SMILES: *c1nnc(C)o1] | CH₃ | 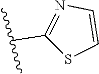 [SMILES: *c1nccs1] | H | H |
| 1080 | 4080 | 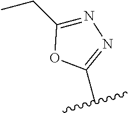 [SMILES: *c1nnc(CC)o1] | CH₃ | 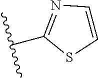 [SMILES: *c1nccs1] | H | H |
| 1081 | 4081 | 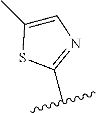 [SMILES: *c1ncc(C)s1] | H | 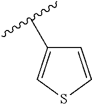 [SMILES: *c1ccsc1] | H | H |
| 1082 | 4082 | 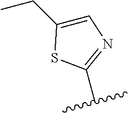 [SMILES: *c1ncc(CC)s1] | H | 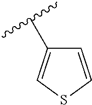 [SMILES: *c1ccsc1] | H | H |
| 1083 | 4083 | 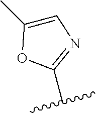 [SMILES: *c1ncc(C)o1] | H | 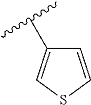 [SMILES: *c1ccsc1] | H | H |
| 1084 | 4084 | 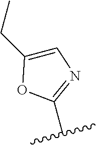 [SMILES: *c1ncc(CC)o1] | H | 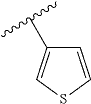 [SMILES: *c1ccsc1] | H | H |
| 1085 | 4085 | 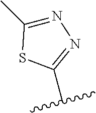 [SMILES: *c1nnc(C)s1] | H | 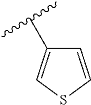 [SMILES: *c1ccsc1] | H | H |
| 1086 | 4086 | 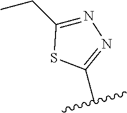 [SMILES: *c1nnc(CC)s1] | H | 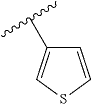 [SMILES: *c1ccsc1] | H | H |
| 1087 | 4087 | 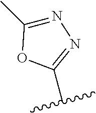 [SMILES: *c1nnc(C)o1] | H | 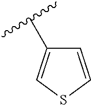 [SMILES: *c1ccsc1] | H | H |
| 1088 | 4088 | 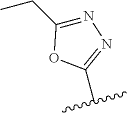 [SMILES: *c1nnc(CC)o1] | H | 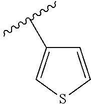 [SMILES: *c1ccsc1] | H | H |
| 1089 | 4089 | 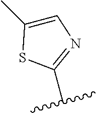 [SMILES: *c1ncc(C)s1] | CH₃ | 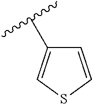 [SMILES: *c1ccsc1] | H | H |
| 1090 | 4090 | 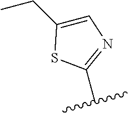 [SMILES: *c1ncc(CC)s1] | CH₃ | 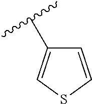 [SMILES: *c1ccsc1] | H | H |
| 1091 | 4091 | 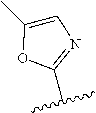 [SMILES: *c1ncc(C)o1] | CH₃ | 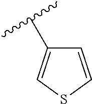 [SMILES: *c1ccsc1] | H | H |
| 1092 | 4092 | 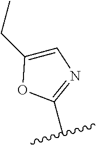 [SMILES: *c1ncc(CC)o1] | CH₃ | 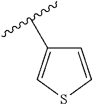 [SMILES: *c1ccsc1] | H | H |
| 1093 | 4093 | 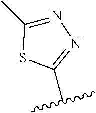 [SMILES: *c1nnc(C)s1] | CH₃ | 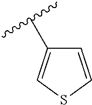 [SMILES: *c1ccsc1] | H | H |
| 1094 | 4094 | 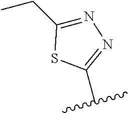 [SMILES: *c1nnc(CC)s1] | CH₃ | 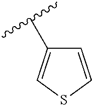 [SMILES: *c1ccsc1] | H | H |
| 1095 | 4095 | 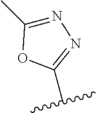 [SMILES: *c1nnc(C)o1] | CH₃ | 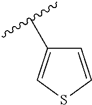 [SMILES: *c1ccsc1] | H | H |
| 1096 | 4096 | 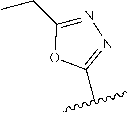 [SMILES: *c1nnc(CC)o1] | CH₃ | 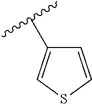 [SMILES: *c1ccsc1] | H | H |
| 1097 | 4097 | 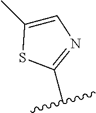 [SMILES: *c1ncc(C)s1] | H | 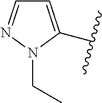 [SMILES: *c1ccnn1CC] | H | F |
| 1098 | 4098 | 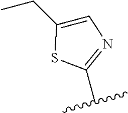 [SMILES: *c1ncc(CC)s1] | H | 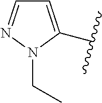 [SMILES: *c1ccnn1CC] | H | F |
| 1099 | 4099 | 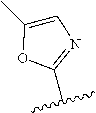 [SMILES: *c1ncc(C)o1] | H | 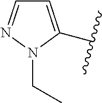 [SMILES: *c1ccnn1CC] | H | F |
| 1100 | 4100 | 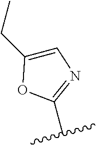 [SMILES: *c1ncc(CC)o1] | H | 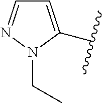 [SMILES: *c1ccnn1CC] | H | F |
| 1101 | 4101 | 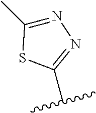 [SMILES: *c1nnc(C)s1] | H | 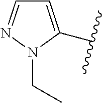 [SMILES: *c1ccnn1CC] | H | F |
| 1102 | 4102 | 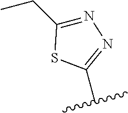 [SMILES: *c1nnc(CC)s1] | H | 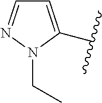 [SMILES: *c1ccnn1CC] | H | F |
| 1103 | 4103 | 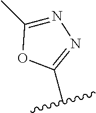 [SMILES: *c1nnc(C)o1] | H | 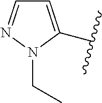 [SMILES: *c1ccnn1CC] | H | F |
| 1104 | 4104 | 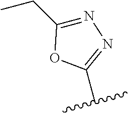 [SMILES: *c1nnc(CC)o1] | H | 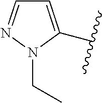 [SMILES: *c1ccnn1CC] | H | F |
| 1105 | 4105 | 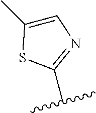 [SMILES: *c1ncc(C)s1] | CH | 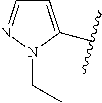 [SMILES: *c1ccnn1CC] | H | F |
| 1106 | 4106 | 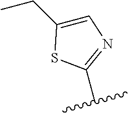 [SMILES: *c1ncc(CC)s1] | CH₃ | 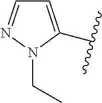 [SMILES: *c1ccnn1CC] | H | F |
| 1107 | 4107 | 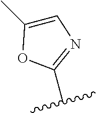 [SMILES: *c1ncc(C)o1] | CH₃ | 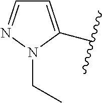 [SMILES: *c1ccnn1CC] | H | F |
| 1108 | 4108 | 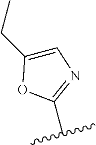 [SMILES: *c1ncc(CC)o1] | CH₃ | 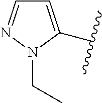 [SMILES: *c1ccnn1CC] | H | F |
| 1109 | 4109 | 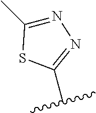 [SMILES: *c1nnc(C)s1] | CH₃ | 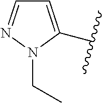 [SMILES: *c1ccnn1CC] | H | F |
| 1110 | 4110 | 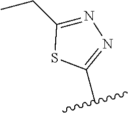 [SMILES: *c1nnc(CC)s1] | CH₃ | 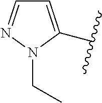 [SMILES: *c1ccnn1CC] | H | F |
| 1111 | 4111 | 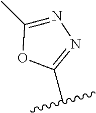 [SMILES: *c1nnc(C)o1] | CH₃ | 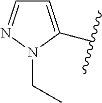 [SMILES: *c1ccnn1CC] | H | F |
| 1112 | 4112 | 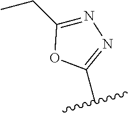 [SMILES: *c1nnc(CC)o1] | CH₃ | 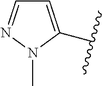 [SMILES: *c1ccnn1C] | I | F |
| 1113 | 4113 | 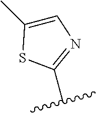 [SMILES: *c1ncc(C)s1] | H | 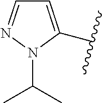 [SMILES: *c1ccnn1C(C)C] | H | F |
| 1114 | 4114 | 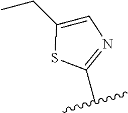 [SMILES: *c1ncc(CC)s1] | H | 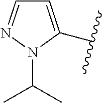 [SMILES: *c1ccnn1C(C)C] | H | F |
| 1115 | 4115 | 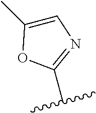 [SMILES: *c1ncc(C)o1] | H | 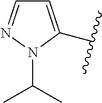 [SMILES: *c1ccnn1C(C)C] | H | F |
| 1116 | 4116 | 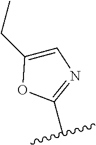 [SMILES: *c1ncc(CC)o1] | H | 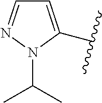 [SMILES: *c1ccnn1C(C)C] | H | F |
| 1117 | 4117 | 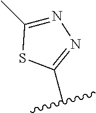 [SMILES: *c1nnc(C)s1] | T | 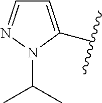 [SMILES: *c1ccnn1C(C)C] | H | F |
| 1118 | 4118 | 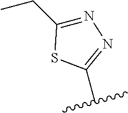 [SMILES: *c1nnc(CC)s1] | H | 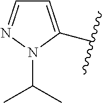 [SMILES: *c1ccnn1C(C)C] | H | F |
| 1119 | 4119 | 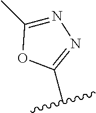 [SMILES: *c1nnc(C)o1] | H | 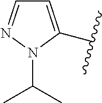 [SMILES: *c1ccnn1C(C)C] | H | F |
| 1120 | 4120 | 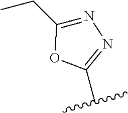 [SMILES: *c1nnc(CC)o1] | H | 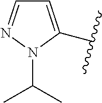 [SMILES: *c1ccnn1C(C)C] | H | F |
| 1121 | 4121 | 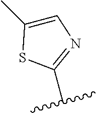 [SMILES: *c1ncc(C)s1] | CH₃ | 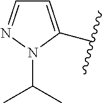 [SMILES: *c1ccnn1C(C)C] | H | F |
| 1122 | 4122 | 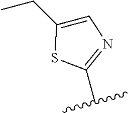 [SMILES: *c1ncc(CC)s1] | CH₃ | 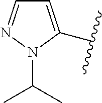 [SMILES: *c1ccnn1C(C)C] | H | F |
| 1123 | 4123 | 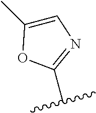 [SMILES: *c1ncc(C)o1] | CH₃ | 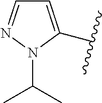 [SMILES: *c1ccnn1C(C)C] | H | F |
| 1124 | 4124 | 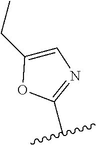 [SMILES: *c1ncc(CC)o1] | CH₃ | 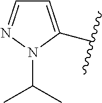 [SMILES: *c1ccnn1C(C)C] | H | F |
| 1125 | 4125 | 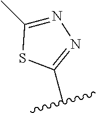 [SMILES: *c1nnc(C)s1] | CH₃ | 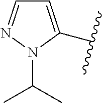 [SMILES: *c1ccnn1C(C)C] | H | F |
| 1126 | 4126 | 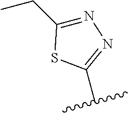 [SMILES: *c1nnc(CC)s1] | CH₃ | 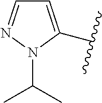 [SMILES: *c1ccnn1C(C)C] | H | F |
| 1127 | 4127 | 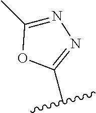 [SMILES: *c1nnc(C)o1] | CH₃ | 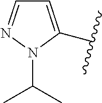 [SMILES: *c1ccnn1C(C)C] | H | F |
| 1128 | 4128 | 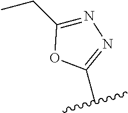 [SMILES: *c1nnc(CC)o1] | CH₃ | 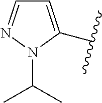 [SMILES: *c1ccnn1C(C)C] | H | F |
| 1129 | 4129 | 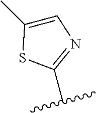 [SMILES: *c1ncc(C)s1] | H | 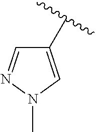 [SMILES: *c1cnn(C)c1] | H | F |
| 1130 | 4130 | 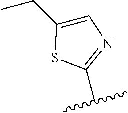 [SMILES: *c1ncc(CC)s1] | H | 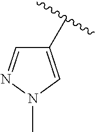 [SMILES: *c1cnn(C)c1] | H | F |
| 1131 | 4131 | 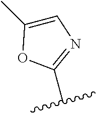 [SMILES: *c1ncc(C)o1] | H | 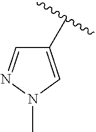 [SMILES: *c1cnn(C)c1] | H | F |
| 1132 | 4132 | 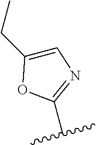 [SMILES: *c1ncc(CC)o1] | H | 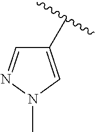 [SMILES: *c1cnn(C)c1] | H | F |
| 1133 | 4133 | 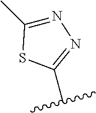 [SMILES: *c1nnc(C)s1] | H | 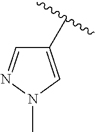 [SMILES: *c1cnn(C)c1] | H | F |
| 1134 | 4134 | 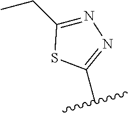 [SMILES: *c1nnc(CC)s1] | H | 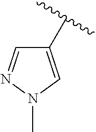 [SMILES: *c1cnn(C)c1] | H | F |
| 1135 | 4135 | 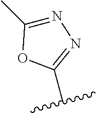 [SMILES: *c1nnc(C)o1] | H | 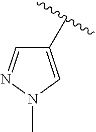 [SMILES: *c1cnn(C)c1] | H | F |
| 1136 | 4136 | 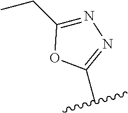 [SMILES: *c1nnc(CC)o1] | H | 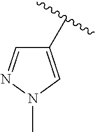 [SMILES: *c1cnn(C)c1] | H | F |
| 1137 | 4137 | 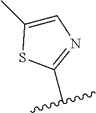 [SMILES: *c1ncc(C)s1] | CH₃ | 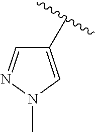 [SMILES: *c1cnn(C)c1] | H | F |
| 1138 | 4138 | 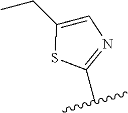 [SMILES: *c1ncc(CC)s1] | CH₃ | 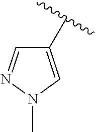 [SMILES: *c1cnn(C)c1] | H | F |
| 1139 | 4139 | 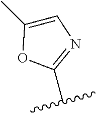 [SMILES: *c1ncc(C)o1] | CH₃ | 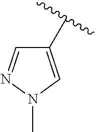 [SMILES: *c1cnn(C)c1] | H | F |
| 1140 | 4140 | 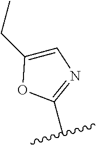 [SMILES: *c1ncc(CC)o1] | CH₃ | 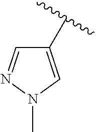 [SMILES: *c1cnn(C)c1] | H | F |
| 1141 | 4141 | 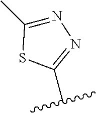 [SMILES: *c1nnc(C)s1] | CH₃ | 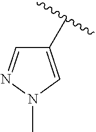 [SMILES: *c1cnn(C)c1] | H | F |
| 1142 | 4142 | 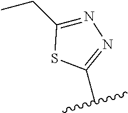 [SMILES: *c1nnc(CC)s1] | CH₃ | 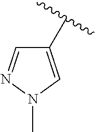 [SMILES: *c1cnn(C)c1] | H | F |
| 1143 | 4143 | 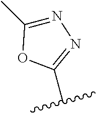 [SMILES: *c1nnc(C)o1] | CH₃ | 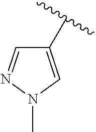 [SMILES: *c1cnn(C)c1] | H | F |
| 1144 | 4144 | 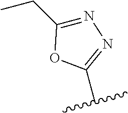 [SMILES: *c1nnc(CC)o1] | CH₃ | 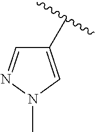 [SMILES: *c1cnn(C)c1] | H | F |
| 1145 | 4145 | 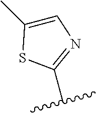 [SMILES: *c1ncc(C)s1] | H | 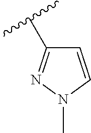 [SMILES: *c1ccn(C)n1] | H | F |
| 1146 | 4146 | 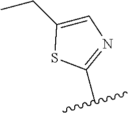 [SMILES: *c1ncc(CC)s1] | H | 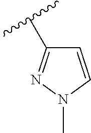 [SMILES: *c1ccn(C)n1] | H | F |
| 1147 | 4147 | 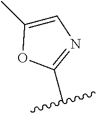 [SMILES: *c1ncc(C)o1] | H | 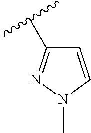 [SMILES: *c1ccn(C)n1] | H | F |
| 1148 | 4148 | 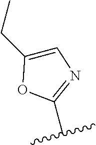 [SMILES: *c1ncc(CC)o1] | H | 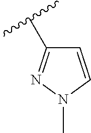 [SMILES: *c1ccn(C)n1] | H | F |
| 1149 | 4149 | 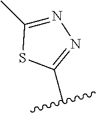 [SMILES: *c1nnc(C)s1] | H | 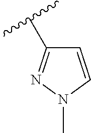 [SMILES: *c1ccn(C)n1] | H | F |
| 1150 | 4150 | 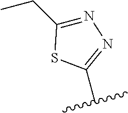 [SMILES: *c1nnc(CC)s1] | H | 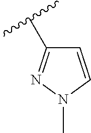 [SMILES: *c1ccn(C)n1] | H | F |
| 1151 | 4151 | 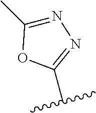 [SMILES: *c1nnc(C)o1] | H | 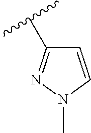 [SMILES: *c1ccn(C)n1] | H | F |
| 1152 | 4152 | 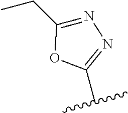 [SMILES: *c1nnc(CC)o1] | H | 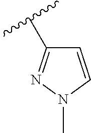 [SMILES: *c1ccn(C)n1] | H | F |
| 1153 | 4153 | 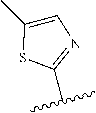 [SMILES: *c1ncc(C)s1] | CH₃ | 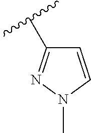 [SMILES: *c1ccn(C)n1] | H | F |
| 1154 | 4154 | 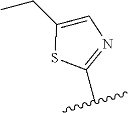 [SMILES: *c1ncc(CC)s1] | CH₃ | 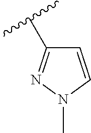 [SMILES: *c1ccn(C)n1] | H | F |
| 1155 | 4155 | 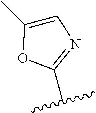 [SMILES: *c1ncc(C)o1] | CH₃ | 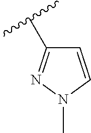 [SMILES: *c1ccn(C)n1] | H | F |
| 1156 | 4156 | 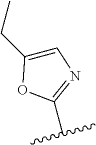 [SMILES: *c1ncc(CC)o1] | CH₃ | 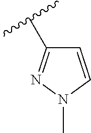 [SMILES: *c1ccn(C)n1] | H | F |
| 1157 | 4157 | 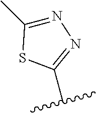 [SMILES: *c1nnc(C)s1] | CH₃ | 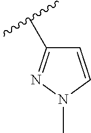 [SMILES: *c1ccn(C)n1] | H | F |
| 1158 | 4158 | 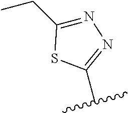 [SMILES: *c1nnc(CC)s1] | CH₃ | 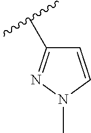 [SMILES: *c1ccn(C)n1] | H | F |
| 1159 | 4159 | 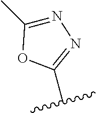 [SMILES: *c1nnc(C)o1] | CH₃ | 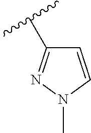 [SMILES: *c1ccn(C)n1] | H | F |
| 1160 | 4160 | 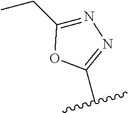 [SMILES: *c1nnc(CC)o1] | CH₃ | 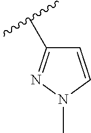 [SMILES: *c1ccn(C)n1] | H | F |
| 1161 | 4161 | 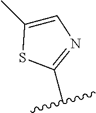 [SMILES: *c1ncc(C)s1] | H | 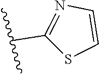 [SMILES: *c1nccs1] | H | F |
| 1162 | 4162 | 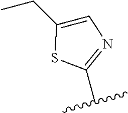 [SMILES: *c1ncc(CC)s1] | H | 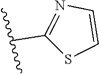 [SMILES: *c1nccs1] | H | F |
| 1163 | 4163 | 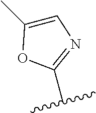 [SMILES: *c1ncc(C)o1] | H | 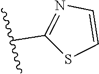 [SMILES: *c1nccs1] | H | F |
| 1164 | 4164 | 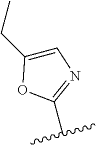 [SMILES: *c1ncc(CC)o1] | H | 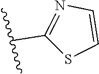 [SMILES: *c1nccs1] | H | F |
| 1165 | 4165 | 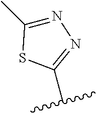 [SMILES: *c1nnc(C)s1] | H | 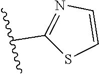 [SMILES: *c1nccs1] | H | F |
| 1166 | 4166 | 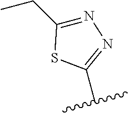 [SMILES: *c1nnc(CC)s1] | H | 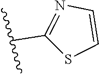 [SMILES: *c1nccs1] | H | F |
| 1167 | 4167 | 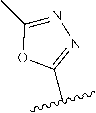 [SMILES: *c1nnc(C)o1] | H | 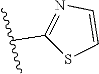 [SMILES: *c1nccs1] | H | F |
| 1168 | 4168 | 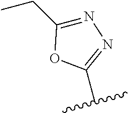 [SMILES: *c1nnc(CC)o1] | H | 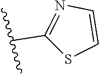 [SMILES: *c1nccs1] | H | F |
| 1169 | 4169 | 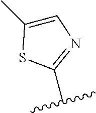 [SMILES: *c1ncc(C)s1] | CH₃ | 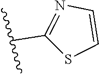 [SMILES: *c1nccs1] | H | F |
| 1170 | 4170 | 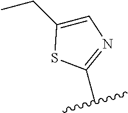 [SMILES: *c1ncc(CC)s1] | CH₃ | 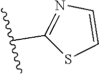 [SMILES: *c1nccs1] | H | F |
| 1171 | 4171 | 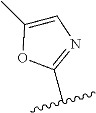 [SMILES: *c1ncc(C)o1] | CH₃ | 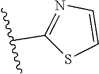 [SMILES: *c1nccs1] | H | F |
| 1172 | 4172 | 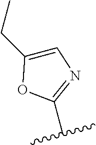 [SMILES: *c1ncc(CC)o1] | CH₃ | 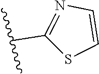 [SMILES: *c1nccs1] | H | F |
| 1173 | 4173 | 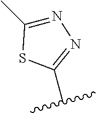 [SMILES: *c1nnc(C)s1] | CH₃ | 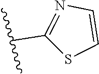 [SMILES: *c1nccs1] | H | F |
| 1174 | 4174 | 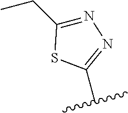 [SMILES: *c1nnc(CC)s1] | CH₃ | 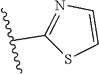 [SMILES: *c1nccs1] | H | F |
| 1175 | 4175 | 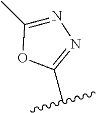 [SMILES: *c1nnc(C)o1] | CH₃ | 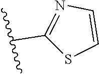 [SMILES: *c1nccs1] | H | F |
| 1176 | 4176 | 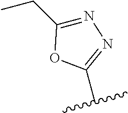 [SMILES: *c1nnc(CC)o1] | CH₃ | 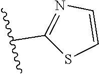 [SMILES: *c1nccs1] | H | F |
| 1177 | 4177 | 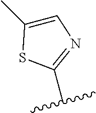 [SMILES: *c1ncc(C)s1] | H | 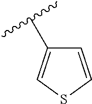 [SMILES: *c1ccsc1] | H | F |
| 1178 | 4178 | 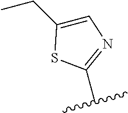 [SMILES: *c1ncc(CC)s1] | H | 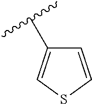 [SMILES: *c1ccsc1] | H | F |
| 1179 | 4179 | 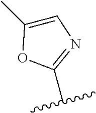 [SMILES: *c1ncc(C)o1] | H | 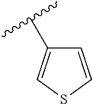 [SMILES: *c1ccsc1] | H | F |
| 1180 | 4180 | 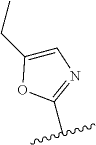 [SMILES: *c1ncc(CC)o1] | H | 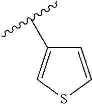 [SMILES: *c1ccsc1] | H | F |
| 1181 | 4181 | 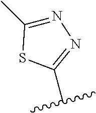 [SMILES: *c1nnc(C)s1] | H | 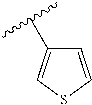 [SMILES: *c1ccsc1] | H | F |
| 1182 | 4182 | 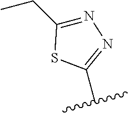 [SMILES: *c1nnc(CC)s1] | H | 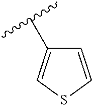 [SMILES: *c1ccsc1] | H | F |
| 1183 | 4183 | 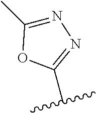 [SMILES: *c1nnc(C)o1] | H | 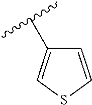 [SMILES: *c1ccsc1] | H | F |
| 1184 | 4184 | 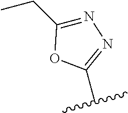 [SMILES: *c1nnc(CC)o1] | H | 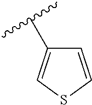 [SMILES: *c1ccsc1] | H | F |
| 1185 | 4185 | 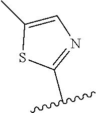 [SMILES: *c1ncc(C)s1] | CH₃ | 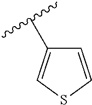 [SMILES: *c1ccsc1] | H | F |
| 1186 | 4186 | 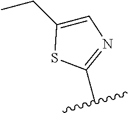 [SMILES: *c1ncc(CC)s1] | CH₃ | 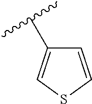 [SMILES: *c1ccsc1] | H | F |
| 1187 | 4187 | 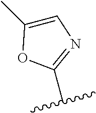 [SMILES: *c1ncc(C)o1] | CH₃ | 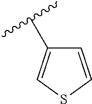 [SMILES: *c1ccsc1] | H | F |
| 1188 | 4188 | 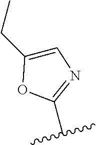 [SMILES: *c1ncc(CC)o1] | CH₃ | 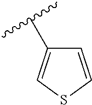 [SMILES: *c1ccsc1] | H | F |
| 1189 | 4189 | 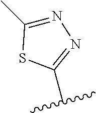 [SMILES: *c1nnc(C)s1] | CH₃ | 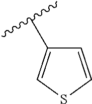 [SMILES: *c1ccsc1] | H | F |
| 1190 | 4190 | 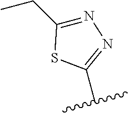 [SMILES: *c1nnc(CC)s1] | CH₃ | 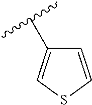 [SMILES: *c1ccsc1] | H | F |
| 1191 | 4191 | 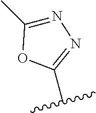 [SMILES: *c1nnc(C)o1] | CH₃ | 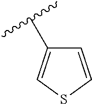 [SMILES: *c1ccsc1] | H | F |
| 1192 | 4192 | 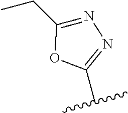 [SMILES: *c1nnc(CC)o1] | CH₃ | 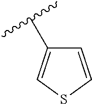 [SMILES: *c1ccsc1] | H | F |
| 1193 | 4193 | 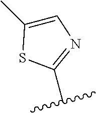 [SMILES: *c1ncc(C)s1] | H | 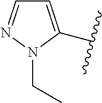 [SMILES: *c1ccnn1CC] | F | H |
| 1194 | 4194 | 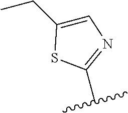 [SMILES: *c1ncc(CC)s1] | H | 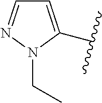 [SMILES: *c1ccnn1CC] | F | H |
| 1195 | 4195 | 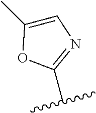 [SMILES: *c1ncc(C)o1] | H | 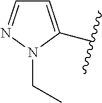 [SMILES: *c1ccnn1CC] | F | H |
| 1196 | 4196 | 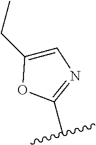 [SMILES: *c1ncc(CC)o1] | H | 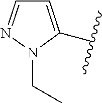 [SMILES: *c1ccnn1CC] | F | H |
| 1197 | 4197 | 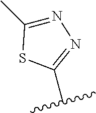 [SMILES: *c1nnc(C)s1] | H | 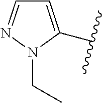 [SMILES: *c1ccnn1CC] | F | H |
| 1198 | 4198 | 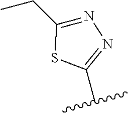 [SMILES: *c1nnc(CC)s1] | H | 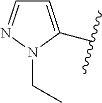 [SMILES: *c1ccnn1CC] | F | H |
| 1199 | 4199 | 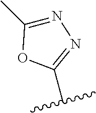 [SMILES: *c1nnc(C)o1] | H | 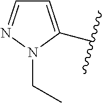 [SMILES: *c1ccnn1CC] | F | H |
| 1200 | 4200 | 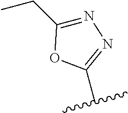 [SMILES: *c1nnc(CC)o1] | H | 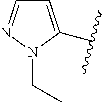 [SMILES: *c1ccnn1CC] | F | H |
| 1201 | 4201 | 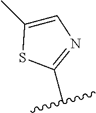 [SMILES: *c1ncc(C)s1] | CH₃ | 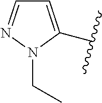 [SMILES: *c1ccnn1CC] | F | H |
| 1202 | 4202 | 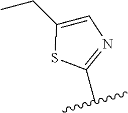 [SMILES: *c1ncc(CC)s1] | CH₃ | 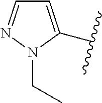 [SMILES: *c1ccnn1CC] | F | H |
| 1203 | 4203 | 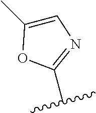 [SMILES: *c1ncc(C)o1] | CH₃ | 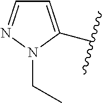 [SMILES: *c1ccnn1CC] | F | H |
| 1204 | 4204 | 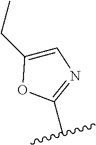 [SMILES: *c1ncc(CC)o1] | CH₃ | 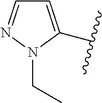 [SMILES: *c1ccnn1CC] | F | H |
| 1205 | 4205 | 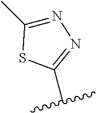 [SMILES: *c1nnc(C)s1] | CH₃ | 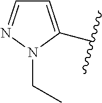 [SMILES: *c1ccnn1CC] | F | H |
| 1206 | 4206 | 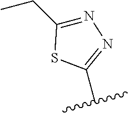 [SMILES: *c1nnc(CC)s1] | CH₃ | 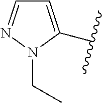 [SMILES: *c1ccnn1CC] | F | H |
| 1207 | 4207 | 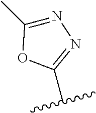 [SMILES: *c1nnc(C)o1] | CH₃ | 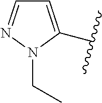 [SMILES: *c1ccnn1CC] | F | H |
| 1208 | 4208 | 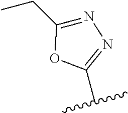 [SMILES: *c1nnc(CC)o1] | CH₃ | 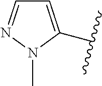 [SMILES: *c1ccnn1C] | F | H |
| 1209 | 4209 | 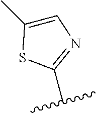 [SMILES: *c1ncc(C)s1] | H | 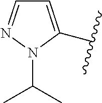 [SMILES: *c1ccnn1C(C)C] | F | H |
| 1210 | 4210 | 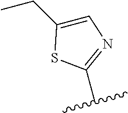 [SMILES: *c1ncc(CC)s1] | H | 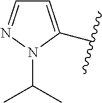 [SMILES: *c1ccnn1C(C)C] | F | H |
| 1211 | 4211 | 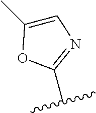 [SMILES: *c1ncc(C)o1] | H | 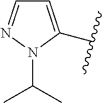 [SMILES: *c1ccnn1C(C)C] | F | H |
| 1212 | 4212 | 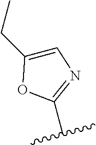 [SMILES: *c1ncc(CC)o1] | H | 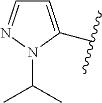 [SMILES: *c1ccnn1C(C)C] | F | H |
| 1213 | 4213 | 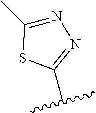 [SMILES: *c1nnc(C)s1] | H | 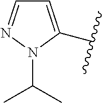 [SMILES: *c1ccnn1C(C)C] | F | H |
| 1214 | 4214 | 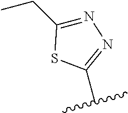 [SMILES: *c1nnc(CC)s1] | H | 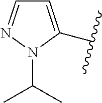 [SMILES: *c1ccnn1C(C)C] | F | H |
| 1215 | 4215 | 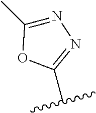 [SMILES: *c1nnc(C)o1] | H | 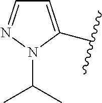 [SMILES: *c1ccnn1C(C)C] | F | H |
| 1216 | 4216 | 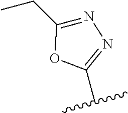 [SMILES: *c1nnc(CC)o1] | H | 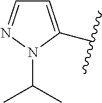 [SMILES: *c1ccnn1C(C)C] | F | H |
| 1217 | 4217 | 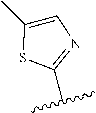 [SMILES: *c1ncc(C)s1] | CH₃ | 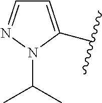 [SMILES: *c1ccnn1C(C)C] | F | H |
| 1218 | 4218 | 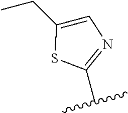 [SMILES: *c1ncc(CC)s1] | CH₃ | 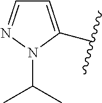 [SMILES: *c1ccnn1C(C)C] | F | H |
| 1219 | 4219 | 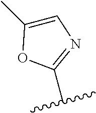 [SMILES: *c1ncc(C)o1] | CH₃ | 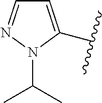 [SMILES: *c1ccnn1C(C)C] | F | H |
| 1220 | 4220 | 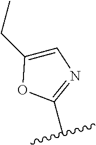 [SMILES: *c1ncc(CC)o1] | CH₃ | 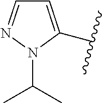 [SMILES: *c1ccnn1C(C)C] | F | H |
| 1221 | 4221 | 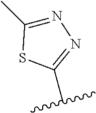 [SMILES: *c1nnc(C)s1] | CH₃ | 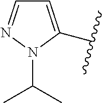 [SMILES: *c1ccnn1C(C)C] | F | H |
| 1222 | 4222 | 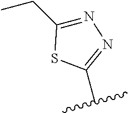 [SMILES: *c1nnc(CC)s1] | CH₃ | 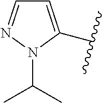 [SMILES: *c1ccnn1C(C)C] | F | H |
| 1223 | 4223 | 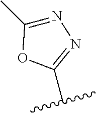 [SMILES: *c1nnc(C)o1] | CH₃ | 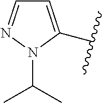 [SMILES: *c1ccnn1C(C)C] | F | H |
| 1224 | 4224 | 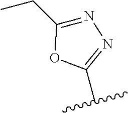 [SMILES: *c1nnc(CC)o1] | CH₃ | 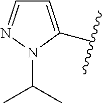 [SMILES: *c1ccnn1C(C)C] | F | H |
| 1225 | 4225 | 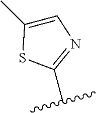 [SMILES: *c1ncc(C)s1] | H | 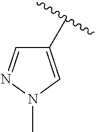 [SMILES: *c1cnn(C)c1] | F | H |
| 1226 | 4226 | 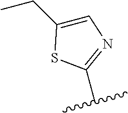 [SMILES: *c1ncc(CC)s1] | H | 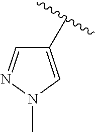 [SMILES: *c1cnn(C)c1] | F | H |
| 1227 | 4227 | 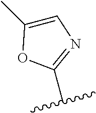 [SMILES: *c1ncc(C)o1] | H | 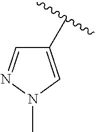 [SMILES: *c1cnn(C)c1] | F | H |
| 1228 | 4228 | 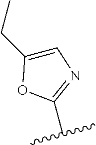 [SMILES: *c1ncc(CC)o1] | H | 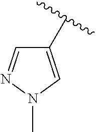 [SMILES: *c1cnn(C)c1] | F | H |
| 1229 | 4229 | 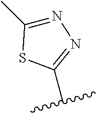 [SMILES: *c1nnc(C)s1] | H | 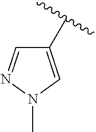 [SMILES: *c1cnn(C)c1] | F | H |
| 1230 | 4230 | 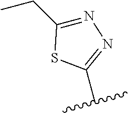 [SMILES: *c1nnc(CC)s1] | H | 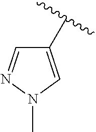 [SMILES: *c1cnn(C)c1] | F | H |
| 1231 | 4231 | 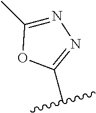 [SMILES: *c1nnc(C)o1] | H | 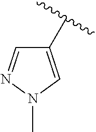 [SMILES: *c1cnn(C)c1] | F | H |
| 1232 | 4232 | 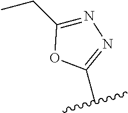 [SMILES: *c1nnc(CC)o1] | H | 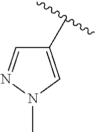 [SMILES: *c1cnn(C)c1] | F | H |
| 1233 | 4233 | 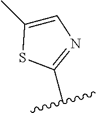 [SMILES: *c1ncc(C)s1] | CH₃ | 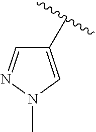 [SMILES: *c1cnn(C)c1] | F | H |
| 1234 | 4234 | 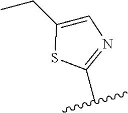 [SMILES: *c1ncc(CC)s1] | CH₃ | 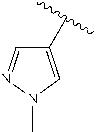 [SMILES: *c1cnn(C)c1] | F | H |
| 1235 | 4235 | 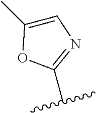 [SMILES: *c1ncc(C)o1] | CH₃ | 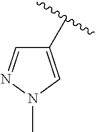 [SMILES: *c1cnn(C)c1] | F | H |
| 1236 | 4236 | 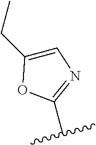 [SMILES: *c1ncc(CC)o1] | CH₃ | 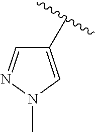 [SMILES: *c1cnn(C)c1] | F | H |
| 1237 | 4237 | 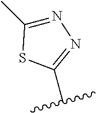 [SMILES: *c1nnc(C)s1] | CH₃ | 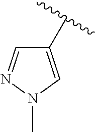 [SMILES: *c1cnn(C)c1] | F | H |
| 1238 | 4238 | 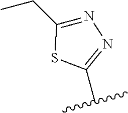 [SMILES: *c1nnc(CC)s1] | CH₃ | 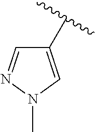 [SMILES: *c1cnn(C)c1] | F | H |
| 1239 | 4239 | 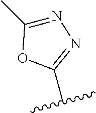 [SMILES: *c1nnc(C)o1] | CH₃ | 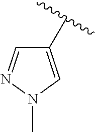 [SMILES: *c1cnn(C)c1] | F | H |
| 1240 | 4240 | 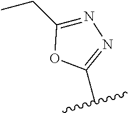 [SMILES: *c1nnc(CC)o1] | CH₃ | 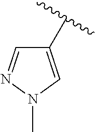 [SMILES: *c1cnn(C)c1] | F | H |
| 1241 | 4241 | 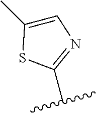 [SMILES: *c1ncc(C)s1] | H | 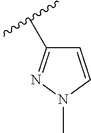 [SMILES: *c1ccn(C)n1] | F | H |
| 1242 | 4242 | 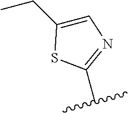 [SMILES: *c1ncc(CC)s1] | H | 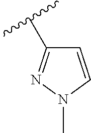 [SMILES: *c1ccn(C)n1] | F | H |
| 1243 | 4243 | 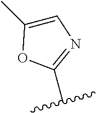 [SMILES: *c1ncc(C)o1] | H | 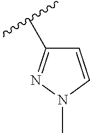 [SMILES: *c1ccn(C)n1] | F | H |
| 1244 | 4244 | 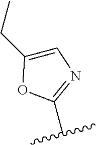 [SMILES: *c1ncc(CC)o1] | H | 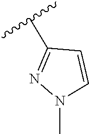 [SMILES: *c1ccn(C)n1] | F | H |
| 1245 | 4245 | 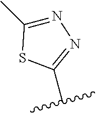 [SMILES: *c1nnc(C)s1] | H | 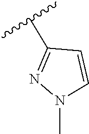 [SMILES: *c1ccn(C)n1] | F | H |
| 1246 | 4246 | 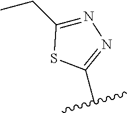 [SMILES: *c1nnc(CC)s1] | H | 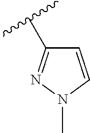 [SMILES: *c1ccn(C)n1] | F | H |
| 1247 | 4247 | 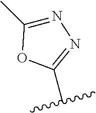 [SMILES: *c1nnc(C)o1] | H | 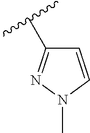 [SMILES: *c1ccn(C)n1] | F | H |
| 1248 | 4248 | 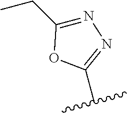 [SMILES: *c1nnc(CC)o1] | H | 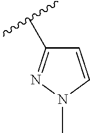 [SMILES: *c1ccn(C)n1] | F | H |
| 1249 | 4249 | 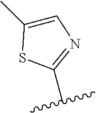 [SMILES: *c1ncc(C)s1] | CH₃ | 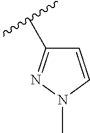 [SMILES: *c1ccn(C)n1] | F | H |
| 1250 | 4250 | 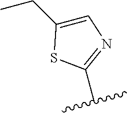 [SMILES: *c1ncc(CC)s1] | CH₃ | 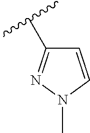 [SMILES: *c1ccn(C)n1] | F | H |
| 1251 | 4251 | 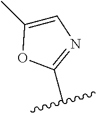 [SMILES: *c1ncc(C)o1] | CH₃ | 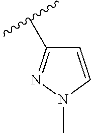 [SMILES: *c1ccn(C)n1] | F | H |
| 1252 | 4252 | 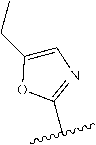 [SMILES: *c1ncc(CC)o1] | CH₃ | 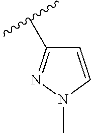 [SMILES: *c1ccn(C)n1] | F | H |
| 1253 | 4253 | 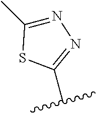 [SMILES: *c1nnc(C)s1] | CH₃ | 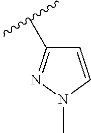 [SMILES: *c1ccn(C)n1] | F | H |
| 1254 | 4254 | 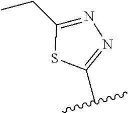 [SMILES: *c1nnc(CC)s1] | CH₃ | 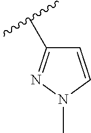 [SMILES: *c1ccn(C)n1] | F | H |
| 1255 | 4255 | 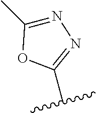 [SMILES: *c1nnc(C)o1] | CH₃ | 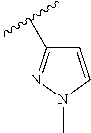 [SMILES: *c1ccn(C)n1] | F | H |
| 1256 | 4256 | 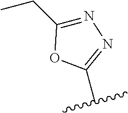 [SMILES: *c1nnc(CC)o1] | CH₃ | 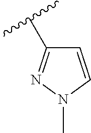 [SMILES: *c1ccn(C)n1] | F | H |
| 1257 | 4257 | 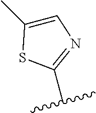 [SMILES: *c1ncc(C)s1] | H | 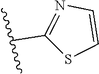 [SMILES: *c1nccs1] | F | H |
| 1258 | 4258 | 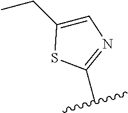 [SMILES: *c1ncc(CC)s1] | H | 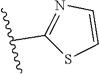 [SMILES: *c1nccs1] | F | H |
| 1259 | 4259 | 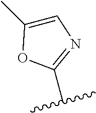 [SMILES: *c1ncc(C)o1] | H | 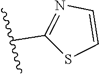 [SMILES: *c1nccs1] | F | H |
| 1260 | 4260 | 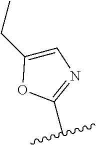 [SMILES: *c1ncc(CC)o1] | H | 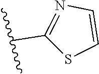 [SMILES: *c1nccs1] | F | H |
| 1261 | 4261 | 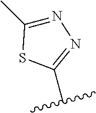 [SMILES: *c1nnc(C)s1] | H | 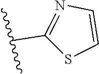 [SMILES: *c1nccs1] | F | H |
| 1262 | 4262 | 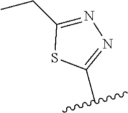 [SMILES: *c1nnc(CC)s1] | H | 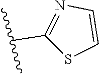 [SMILES: *c1nccs1] | F | H |
| 1263 | 4263 | 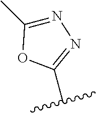 [SMILES: *c1nnc(C)o1] | H | 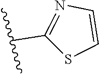 [SMILES: *c1nccs1] | F | H |
| 1264 | 4264 | 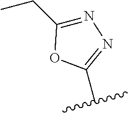 [SMILES: *c1nnc(CC)o1] | H | 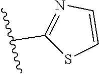 [SMILES: *c1nccs1] | F | H |
| 1265 | 4265 | 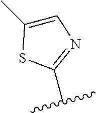 [SMILES: *c1ncc(C)s1] | CH₃ | 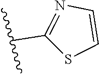 [SMILES: *c1nccs1] | F | H |
| 1266 | 4266 | 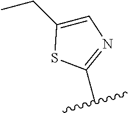 [SMILES: *c1ncc(CC)s1] | CH₃ | 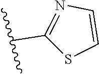 [SMILES: *c1nccs1] | F | H |
| 1267 | 4267 | 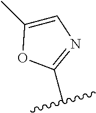 [SMILES: *c1ncc(C)o1] | CH₃ | 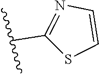 [SMILES: *c1nccs1] | F | H |
| 1268 | 4268 | 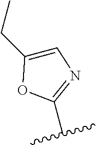 [SMILES: *c1ncc(CC)o1] | CH₃ | 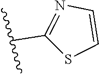 [SMILES: *c1nccs1] | F | H |
| 1269 | 4269 | 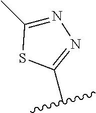 [SMILES: *c1nnc(C)s1] | CH₃ | 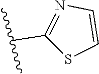 [SMILES: *c1nccs1] | F | H |
| 1270 | 4270 | 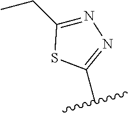 [SMILES: *c1nnc(CC)s1] | CH₃ | 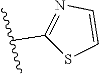 [SMILES: *c1nccs1] | F | H |
| 1271 | 4271 | 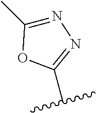 [SMILES: *c1nnc(C)o1] | CH₃ | 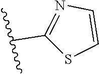 [SMILES: *c1nccs1] | F | H |
| 1272 | 4272 | 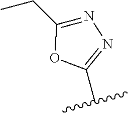 [SMILES: *c1nnc(CC)o1] | CH₃ | 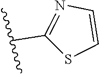 [SMILES: *c1nccs1] | F | H |
| 1273 | 4273 | 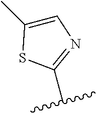 [SMILES: *c1ncc(C)s1] | H | 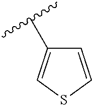 [SMILES: *c1ccsc1] | F | H |
| 1274 | 4274 | 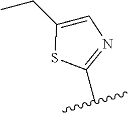 [SMILES: *c1ncc(CC)s1] | H | 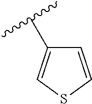 [SMILES: *c1ccsc1] | F | H |
| 1275 | 4275 | 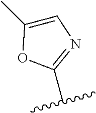 [SMILES: *c1ncc(C)o1] | H | 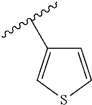 [SMILES: *c1ccsc1] | F | H |
| 1276 | 4276 | 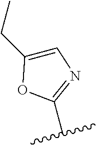 [SMILES: *c1ncc(CC)o1] | H | 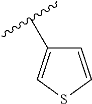 [SMILES: *c1ccsc1] | F | H |
| 1277 | 4277 | 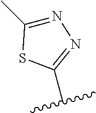 [SMILES: *c1nnc(C)s1] | H | 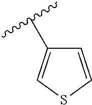 [SMILES: *c1ccsc1] | F | H |
| 1278 | 4278 | 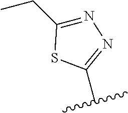 [SMILES: *c1nnc(CC)s1] | H | 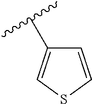 [SMILES: *c1ccsc1] | F | H |
| 1279 | 4279 | 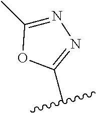 [SMILES: *c1nnc(C)o1] | H | 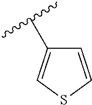 [SMILES: *c1ccsc1] | F | H |
| 1280 | 4280 | 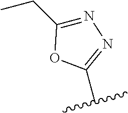 [SMILES: *c1nnc(CC)o1] | H | 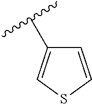 [SMILES: *c1ccsc1] | F | H |
| 1281 | 4281 | 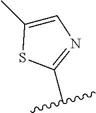 [SMILES: *c1ncc(C)s1] | CH₃ | 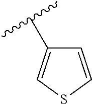 [SMILES: *c1ccsc1] | F | H |
| 1282 | 4282 | 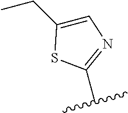 [SMILES: *c1ncc(CC)s1] | CH₃ | 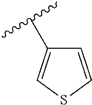 [SMILES: *c1ccsc1] | F | H |
| 1283 | 4283 | 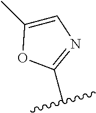 [SMILES: *c1ncc(C)o1] | CH₃ | 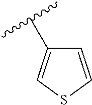 [SMILES: *c1ccsc1] | F | H |
| 1284 | 4284 | 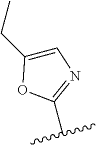 [SMILES: *c1ncc(CC)o1] | CH₃ | 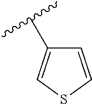 [SMILES: *c1ccsc1] | F | H |
| 1285 | 4285 | 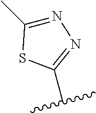 [SMILES: *c1nnc(C)s1] | CH₃ | 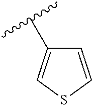 [SMILES: *c1ccsc1] | F | H |
| 1286 | 4286 | 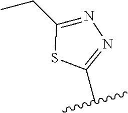 [SMILES: *c1nnc(CC)s1] | CH₃ | 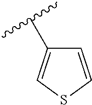 [SMILES: *c1ccsc1] | F | H |
| 1287 | 4287 | 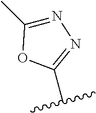 [SMILES: *c1nnc(C)o1] | CH₃ | 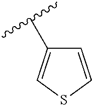 [SMILES: *c1ccsc1] | F | H |
| 1288 | 4288 | 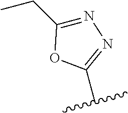 [SMILES: *c1nnc(CC)o1] | CH₃ | 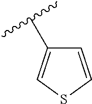 [SMILES: *c1ccsc1] | F | H |
| 1289 | 4289 | 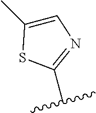 [SMILES: *c1ncc(C)s1] | H | 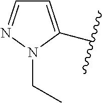 [SMILES: *c1ccnn1CC] | H | Cl |
| 1290 | 4290 | 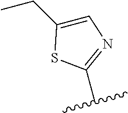 [SMILES: *c1ncc(CC)s1] | H | 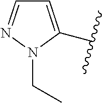 [SMILES: *c1ccnn1CC] | H | Cl |
| 1291 | 4291 | 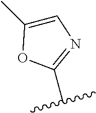 [SMILES: *c1ncc(C)o1] | H | 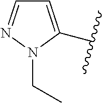 [SMILES: *c1ccnn1CC] | H | Cl |
| 1292 | 4292 | 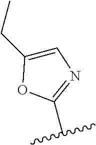 [SMILES: *c1ncc(CC)o1] | H | 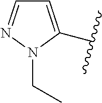 [SMILES: *c1ccnn1CC] | H | Cl |
| 1293 | 4293 | 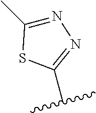 [SMILES: *c1nnc(C)s1] | H | 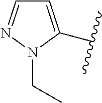 [SMILES: *c1ccnn1CC] | H | Cl |
| 1294 | 4294 | 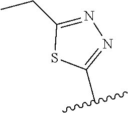 [SMILES: *c1nnc(CC)s1] | H | 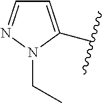 [SMILES: *c1ccnn1CC] | H | Cl |
| 1295 | 4295 | 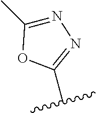 [SMILES: *c1nnc(C)o1] | H | 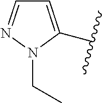 [SMILES: *c1ccnn1CC] | H | Cl |
| 1296 | 4296 | 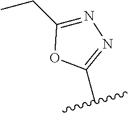 [SMILES: *c1nnc(CC)o1] | H | 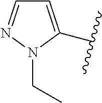 [SMILES: *c1ccnn1CC] | H | Cl |
| 1297 | 4297 | 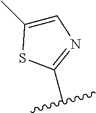 [SMILES: *c1ncc(C)s1] | CH₃ | 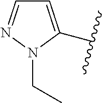 [SMILES: *c1ccnn1CC] | H | Cl |
| 1298 | 4298 | 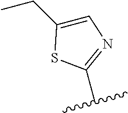 [SMILES: *c1ncc(CC)s1] | CH₃ | 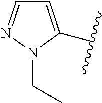 [SMILES: *c1ccnn1CC] | H | Cl |
| 1299 | 4299 | 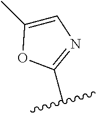 [SMILES: *c1ncc(C)o1] | CH₃ | 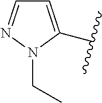 [SMILES: *c1ccnn1CC] | H | Cl |
| 1300 | 4300 | 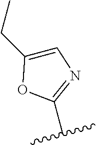 [SMILES: *c1ncc(CC)o1] | CH₃ | 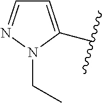 [SMILES: *c1ccnn1CC] | H | Cl |
| 1301 | 4301 | 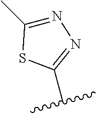 [SMILES: *c1nnc(C)s1] | CH₃ | 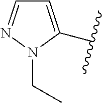 [SMILES: *c1ccnn1CC] | H | Cl |
| 1302 | 4302 | 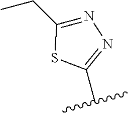 [SMILES: *c1nnc(CC)s1] | CH₃ | 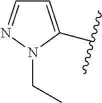 [SMILES: *c1ccnn1CC] | H | Cl |
| 1303 | 4303 | 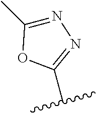 [SMILES: *c1nnc(C)o1] | CH₃ | 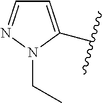 [SMILES: *c1ccnn1CC] | H | Cl |
| 1304 | 4304 | 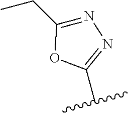 [SMILES: *c1nnc(CC)o1] | CH₃ | 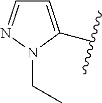 [SMILES: *c1ccnn1CC] | H | Cl |
| 1305 | 4305 | 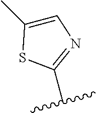 [SMILES: *c1ncc(C)s1] | H | 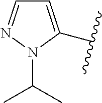 [SMILES: *c1ccnn1C(C)C] | H | Cl |
| 1306 | 4306 | 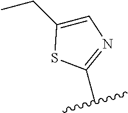 [SMILES: *c1ncc(CC)s1] | H | 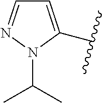 [SMILES: *c1ccnn1C(C)C] | H | Cl |
| 1307 | 4307 | 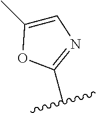 [SMILES: *c1ncc(C)o1] | H | 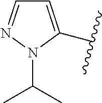 [SMILES: *c1ccnn1C(C)C] | H | Cl |
| 1308 | 4308 | 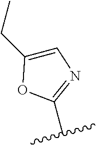 [SMILES: *c1ncc(CC)o1] | H | 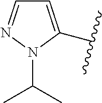 [SMILES: *c1ccnn1C(C)C] | H | Cl |
| 1309 | 4309 | 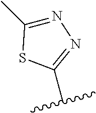 [SMILES: *c1nnc(C)s1] | H | 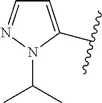 [SMILES: *c1ccnn1C(C)C] | H | Cl |
| 1310 | 4310 | 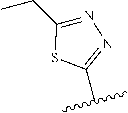 [SMILES: *c1nnc(CC)s1] | H | 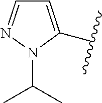 [SMILES: *c1ccnn1C(C)C] | H | Cl |
| 1311 | 4311 | 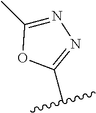 [SMILES: *c1nnc(C)o1] | H | 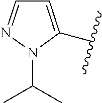 [SMILES: *c1ccnn1C(C)C] | H | Cl |
| 1312 | 4312 | 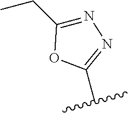 [SMILES: *c1nnc(CC)o1] | H | 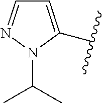 [SMILES: *c1ccnn1C(C)C] | H | Cl |
| 1313 | 4313 | 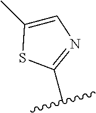 [SMILES: *c1ncc(C)s1] | CH₃ | 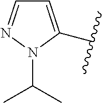 [SMILES: *c1ccnn1C(C)C] | H | Cl |
| 1314 | 4314 | 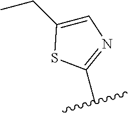 [SMILES: *c1ncc(CC)s1] | CH₃ | 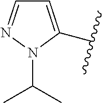 [SMILES: *c1ccnn1C(C)C] | H | Cl |
| 1315 | 4315 | 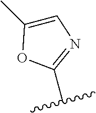 [SMILES: *c1ncc(C)o1] | CH₃ | 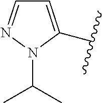 [SMILES: *c1ccnn1C(C)C] | H | Cl |
| 1316 | 4316 | 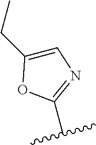 [SMILES: *c1ncc(CC)o1] | CH₃ | 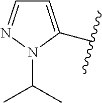 [SMILES: *c1ccnn1C(C)C] | H | Cl |
| 1317 | 4317 | 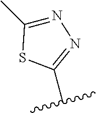 [SMILES: *c1nnc(C)s1] | CH₃ | 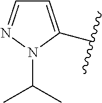 [SMILES: *c1ccnn1C(C)C] | H | Cl |
| 1318 | 4318 | 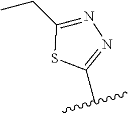 [SMILES: *c1nnc(CC)s1] | CH₃ | 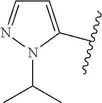 [SMILES: *c1ccnn1C(C)C] | H | Cl |
| 1319 | 4319 | 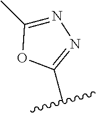 [SMILES: *c1nnc(C)o1] | CH₃ | 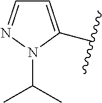 [SMILES: *c1ccnn1C(C)C] | H | Cl |
| 1320 | 4320 | 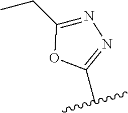 [SMILES: *c1nnc(CC)o1] | CH₃ | 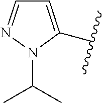 [SMILES: *c1ccnn1C(C)C] | H | Cl |
| 1321 | 4321 | 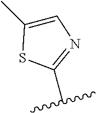 [SMILES: *c1ncc(C)s1] | H | 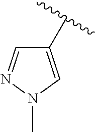 [SMILES: *c1cnn(C)c1] | H | Cl |
| 1322 | 4322 | 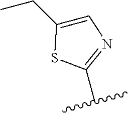 [SMILES: *c1ncc(CC)s1] | H | 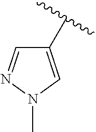 [SMILES: *c1cnn(C)c1] | H | Cl |
| 1323 | 4323 | 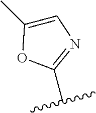 [SMILES: *c1ncc(C)o1] | H | 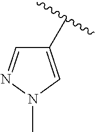 [SMILES: *c1cnn(C)c1] | H | Cl |
| 1324 | 4324 | 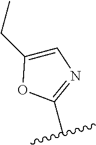 [SMILES: *c1ncc(CC)o1] | H | 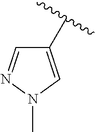 [SMILES: *c1cnn(C)c1] | H | Cl |
| 1325 | 4325 | 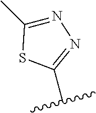 [SMILES: *c1nnc(C)s1] | H | 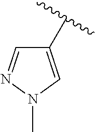 [SMILES: *c1cnn(C)c1] | H | Cl |
| 1326 | 4326 | 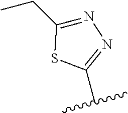 [SMILES: *c1nnc(CC)s1] | H | 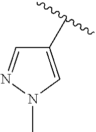 [SMILES: *c1cnn(C)c1] | H | Cl |
| 1327 | 4327 | 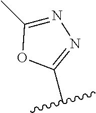 [SMILES: *c1nnc(C)o1] | H | 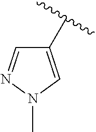 [SMILES: *c1cnn(C)c1] | H | Cl |
| 1328 | 4328 | 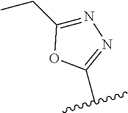 [SMILES: *c1nnc(CC)o1] | H | 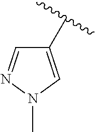 [SMILES: *c1cnn(C)c1] | H | Cl |
| 1329 | 4329 | 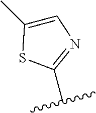 [SMILES: *c1ncc(C)s1] | CH₃ | 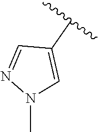 [SMILES: *c1cnn(C)c1] | H | Cl |
| 1330 | 4330 | 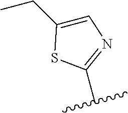 [SMILES: *c1ncc(CC)s1] | CH₃ | 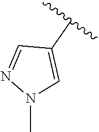 [SMILES: *c1cnn(C)c1] | H | Cl |
| 1331 | 4331 | 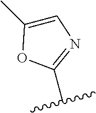 [SMILES: *c1ncc(C)o1] | CH₃ | 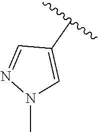 [SMILES: *c1cnn(C)c1] | H | Cl |
| 1332 | 4332 | 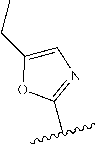 [SMILES: *c1ncc(CC)o1] | CH₃ | 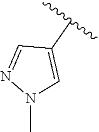 [SMILES: *c1cnn(C)c1] | H | Cl |
| 1333 | 4333 | 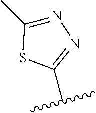 [SMILES: *c1nnc(C)s1] | CH₃ | 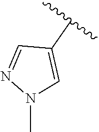 [SMILES: *c1cnn(C)c1] | H | Cl |
| 1334 | 4334 | 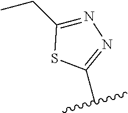 [SMILES: *c1nnc(CC)s1] | CH₃ | 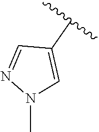 [SMILES: *c1cnn(C)c1] | H | Cl |
| 1335 | 4335 | 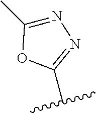 [SMILES: *c1nnc(C)o1] | CH₃ | 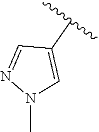 [SMILES: *c1cnn(C)c1] | H | Cl |
| 1336 | 4336 | 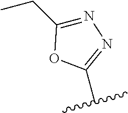 [SMILES: *c1nnc(CC)o1] | CH₃ | 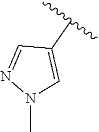 [SMILES: *c1cnn(C)c1] | H | Cl |
| 1337 | 4337 | 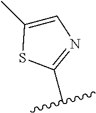 [SMILES: *c1ncc(C)s1] | H | 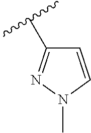 [SMILES: *c1ccn(C)n1] | H | Cl |
| 1338 | 4338 | 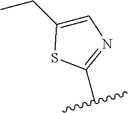 [SMILES: *c1ncc(CC)s1] | H | 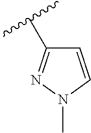 [SMILES: *c1ccn(C)n1] | H | Cl |
| 1339 | 4339 | 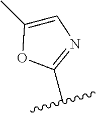 [SMILES: *c1ncc(C)o1] | H | 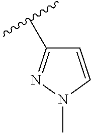 [SMILES: *c1ccn(C)n1] | H | Cl |
| 1340 | 4340 | 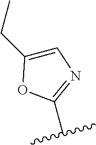 [SMILES: *c1ncc(CC)o1] | H | 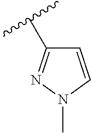 [SMILES: *c1ccn(C)n1] | H | Cl |
| 1341 | 4341 | 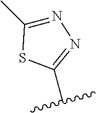 [SMILES: *c1nnc(C)s1] | H | 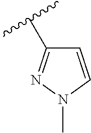 [SMILES: *c1ccn(C)n1] | H | Cl |
| 1342 | 4342 | 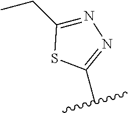 [SMILES: *c1nnc(CC)s1] | H | 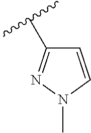 [SMILES: *c1ccn(C)n1] | H | Cl |
| 1343 | 4343 | 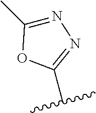 [SMILES: *c1nnc(C)o1] | H | 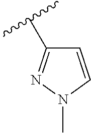 [SMILES: *c1ccn(C)n1] | H | Cl |
| 1344 | 4344 | 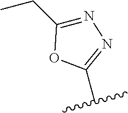 [SMILES: *c1nnc(CC)o1] | H | 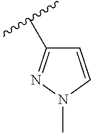 [SMILES: *c1ccn(C)n1] | H | Cl |
| 1345 | 4345 | 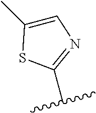 [SMILES: *c1ncc(C)s1] | CH₃ | 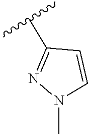 [SMILES: *c1ccn(C)n1] | H | Cl |
| 1346 | 4346 | 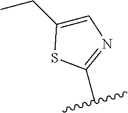 [SMILES: *c1ncc(CC)s1] | CH₃ | 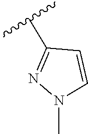 [SMILES: *c1ccn(C)n1] | H | Cl |
| 1347 | 4347 | 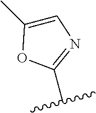 [SMILES: *c1ncc(C)o1] | CH₃ | 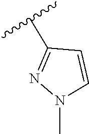 [SMILES: *c1ccn(C)n1] | H | Cl |
| 1348 | 4348 | 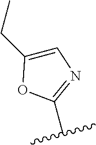 [SMILES: *c1ncc(CC)o1] | CH₃ | 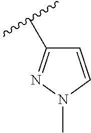 [SMILES: *c1ccn(C)n1] | H | Cl |
| 1349 | 4349 | 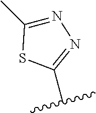 [SMILES: *c1nnc(C)s1] | CH₃ | 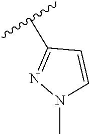 [SMILES: *c1ccn(C)n1] | H | Cl |
| 1350 | 4350 | 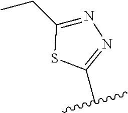 [SMILES: *c1nnc(CC)s1] | CH₃ | 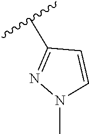 [SMILES: *c1ccn(C)n1] | H | Cl |
| 1351 | 4351 | 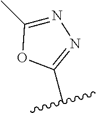 [SMILES: *c1nnc(C)o1] | CH₃ | 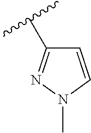 [SMILES: *c1ccn(C)n1] | H | Cl |
| 1352 | 4352 | 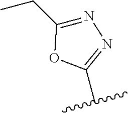 [SMILES: *c1nnc(CC)o1] | CH₃ | 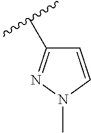 [SMILES: *c1ccn(C)n1] | H | Cl |
| 1353 | 4353 | 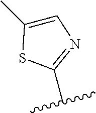 [SMILES: *c1ncc(C)s1] | H | 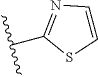 [SMILES: *c1nccs1] | H | Cl |
| 1354 | 4354 | 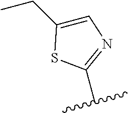 [SMILES: *c1ncc(CC)s1] | H | 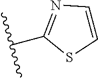 [SMILES: *c1nccs1] | H | Cl |
| 1355 | 4355 | 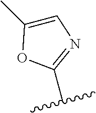 [SMILES: *c1ncc(C)o1] | H | 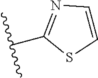 [SMILES: *c1nccs1] | H | Cl |
| 1356 | 4356 | 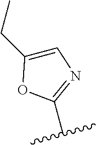 [SMILES: *c1ncc(CC)o1] | H | 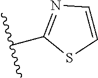 [SMILES: *c1nccs1] | H | Cl |
| 1357 | 4357 | 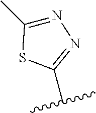 [SMILES: *c1nnc(C)s1] | H | 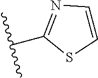 [SMILES: *c1nccs1] | H | Cl |
| 1358 | 4358 | 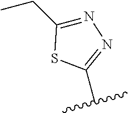 [SMILES: *c1nnc(CC)s1] | H | 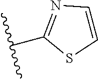 [SMILES: *c1nccs1] | H | Cl |
| 1359 | 4359 | 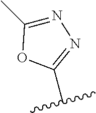 [SMILES: *c1nnc(C)o1] | H | 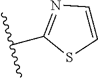 [SMILES: *c1nccs1] | H | Cl |
| 1360 | 4360 | 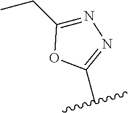 [SMILES: *c1nnc(CC)o1] | H | 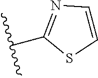 [SMILES: *c1nccs1] | H | Cl |
| 1361 | 4361 | 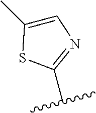 [SMILES: *c1ncc(C)s1] | CH₃ | 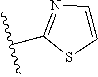 [SMILES: *c1nccs1] | H | Cl |
| 1362 | 4362 | 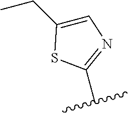 [SMILES: *c1ncc(CC)s1] | CH₃ | 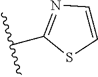 [SMILES: *c1nccs1] | H | Cl |
| 1363 | 4363 | 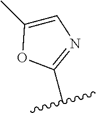 [SMILES: *c1ncc(C)o1] | CH₃ | 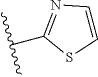 [SMILES: *c1nccs1] | H | Cl |
| 1364 | 4364 | 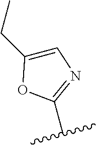 [SMILES: *c1ncc(CC)o1] | CH₃ | 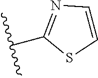 [SMILES: *c1nccs1] | H | Cl |
| 1365 | 4365 | 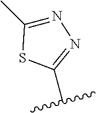 [SMILES: *c1nnc(C)s1] | CH₃ | 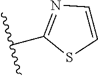 [SMILES: *c1nccs1] | H | Cl |
| 1366 | 4366 | 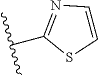 [SMILES: *c1nccs1] | CH₃ | 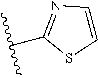 [SMILES: *c1nccs1] | H | Cl |
| 1367 | 4367 | 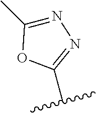 [SMILES: *c1nnc(C)o1] | CH₃ | 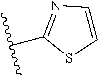 [SMILES: *c1nccs1] | H | Cl |
| 1368 | 4368 | 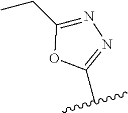 [SMILES: *c1nnc(CC)o1] | CH₃ | 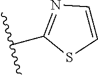 [SMILES: *c1nccs1] | H | Cl |
| 1369 | 4369 | 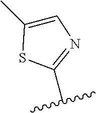 [SMILES: *c1ncc(C)s1] | H | 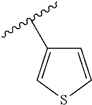 [SMILES: *c1ccsc1] | H | Cl |
| 1370 | 4370 | 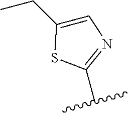 [SMILES: *c1ncc(CC)s1] | H | 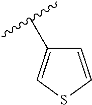 [SMILES: *c1ccsc1] | H | Cl |
| 1371 | 4371 | 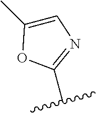 [SMILES: *c1ncc(C)o1] | H | 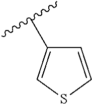 [SMILES: *c1ccsc1] | H | Cl |
| 1372 | 4372 | 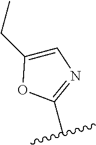 [SMILES: *c1ncc(CC)o1] | H | 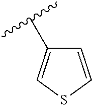 [SMILES: *c1ccsc1] | H | Cl |
| 1373 | 4373 | 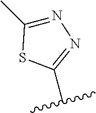 [SMILES: *c1nnc(C)s1] | H | 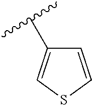 [SMILES: *c1ccsc1] | H | Cl |
| 1374 | 4374 | 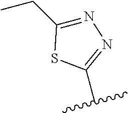 [SMILES: *c1nnc(CC)s1] | H | 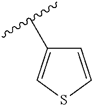 [SMILES: *c1ccsc1] | H | Cl |
| 1375 | 4375 | 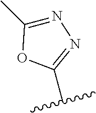 [SMILES: *c1nnc(C)o1] | H | 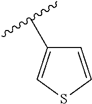 [SMILES: *c1ccsc1] | H | Cl |
| 1376 | 4376 | 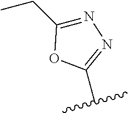 [SMILES: *c1nnc(CC)o1] | H | 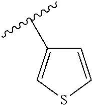 [SMILES: *c1ccsc1] | H | Cl |
| 1377 | 4377 | 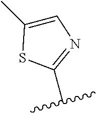 [SMILES: *c1ncc(C)s1] | CH₃ | 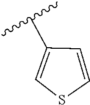 [SMILES: *c1ccsc1] | H | Cl |
| 1378 | 4378 | 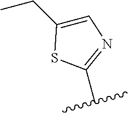 [SMILES: *c1ncc(CC)s1] | CH₃ | 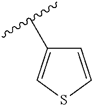 [SMILES: *c1ccsc1] | H | Cl |
| 1379 | 4379 | 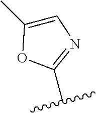 [SMILES: *c1ncc(C)o1] | CH₃ | 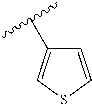 [SMILES: *c1ccsc1] | H | Cl |
| 1380 | 4380 | 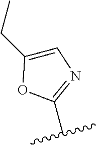 [SMILES: *c1ncc(CC)o1] | CH₃ | 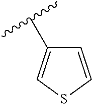 [SMILES: *c1ccsc1] | H | Cl |
| 1381 | 4381 | 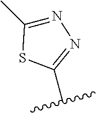 [SMILES: *c1nnc(C)s1] | CH₃ | 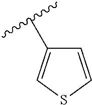 [SMILES: *c1ccsc1] | H | Cl |
| 1382 | 4382 | 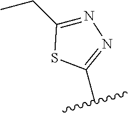 [SMILES: *c1nnc(CC)s1] | CH₃ | 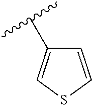 [SMILES: *c1ccsc1] | H | Cl |
| 1383 | 4383 | 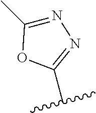 [SMILES: *c1nnc(C)o1] | CH₃ | 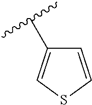 [SMILES: *c1ccsc1] | H | Cl |
| 1384 | 4384 | 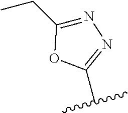 [SMILES: *c1nnc(CC)o1] | CH₃ | 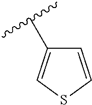 [SMILES: *c1ccsc1] | H | Cl |
| 1385 | 4385 | 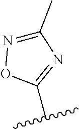 [SMILES: *c1nc(C)no1] | H | 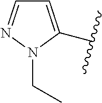 [SMILES: *c1ccnn1CC] | H | H |
| 1386 | 4386 | 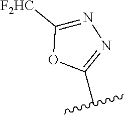 [SMILES: *c1nnc(C(F)F)o1] | H | 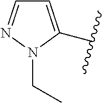 [SMILES: *c1ccnn1CC] | H | H |
| 1387 | 4387 | 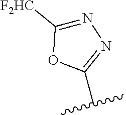 [SMILES: *c1nnc(C(F)F)o1] | H | 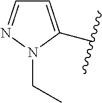 [SMILES: *c1ccnn1CC] | H | H |
| 1388 | 4388 | 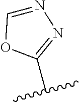 [SMILES: *c1nnco1] | H | 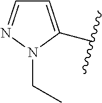 [SMILES: *c1ccnn1CC] | H | H |
| 1389 | 4389 | 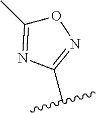 [SMILES: *c1noc(C)n1] | H | 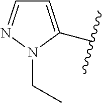 [SMILES: *c1ccnn1CC] | H | H |
| 1390 | 4390 | 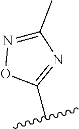 [SMILES: *c1nc(C)no1] | CH₃ | 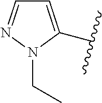 [SMILES: *c1ccnn1CC] | H | H |
| 1391 | 4391 | 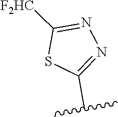 [SMILES: *c1nnc(C(F)F)s1] | CH₃ | 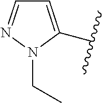 [SMILES: *c1ccnn1CC] | H | H |
| 1392 | 4392 | 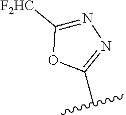 [SMILES: *c1nnc(C(F)F)o1] | CH₃ | 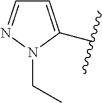 [SMILES: *c1ccnn1CC] | H | H |
| 1393 | 4393 | 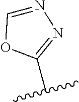 [SMILES: *c1nnco1] | CH₃ | 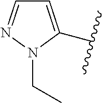 [SMILES: *c1ccnn1CC] | H | H |
| 1394 | 4394 | 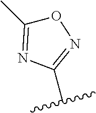 [SMILES: *c1noc(C)n1] | CH₃ | 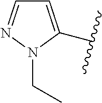 [SMILES: *c1ccnn1CC] | H | H |
| 1395 | 4395 | 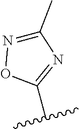 [SMILES: *c1nc(C)no1] | H | 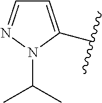 [SMILES: *c1ccnn1C(C)C] | H | H |
| 1396 | 4396 | 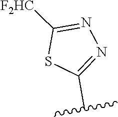 [SMILES: *c1nnc(C(F)F)s1] | H | 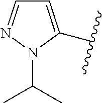 [SMILES: *c1ccnn1C(C)C] | H | H |
| 1397 | 4397 | 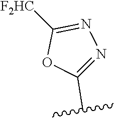 [SMILES: *c1nnc(C(F)F)o1] | H | 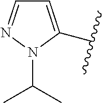 [SMILES: *c1ccnn1C(C)C] | H | H |
| 1398 | 4398 | 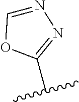 [SMILES: *c1nnco1] | H | 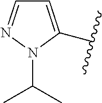 [SMILES: *c1ccnn1C(C)C] | H | H |
| 1399 | 4399 | 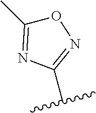 [SMILES: *c1noc(C)n1] | H | 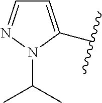 [SMILES: *c1ccnn1C(C)C] | H | H |
| 1400 | 4400 | 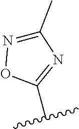 [SMILES: *c1nc(C)no1] | CH₃ | 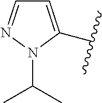 [SMILES: *c1ccnn1C(C)C] | H | H |
| 1401 | 4401 | 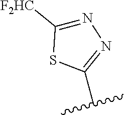 [SMILES: *c1nnc(C(F)F)s1] | CH₃ | 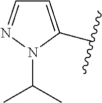 [SMILES: *c1ccnn1C(C)C] | H | H |
| 1402 | 4402 | 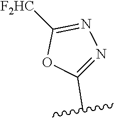 [SMILES: *c1nnc(C(F)F)o1] | CH₃ | 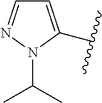 [SMILES: *c1ccnn1C(C)C] | H | H |
| 1403 | 4403 | 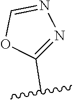 [SMILES: *c1nnco1] | CH₃ | 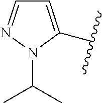 [SMILES: *c1ccnn1C(C)C] | H | H |
| 1404 | 4404 | 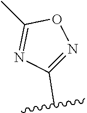 [SMILES: *c1noc(C)n1] | CH₃ | 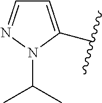 [SMILES: *c1ccnn1C(C)C] | H | H |
| 1405 | 4405 | 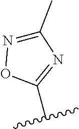 [SMILES: *c1nc(C)no1] | H | 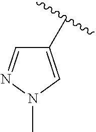 [SMILES: *c1cnn(C)c1] | H | H |
| 1406 | 4406 | 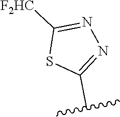 [SMILES: *c1nnc(C(F)F)s1] | H | 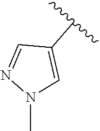 [SMILES: *c1cnn(C)c1] | H | H |
| 1407 | 4407 | 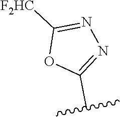 [SMILES: *c1nnc(C(F)F)o1] | H | 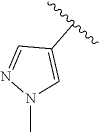 [SMILES: *c1cnn(C)c1] | H | H |
| 1408 | 4408 | 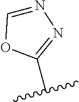 [SMILES: *c1nnco1] | H | 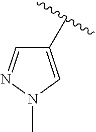 [SMILES: *c1cnn(C)c1] | H | H |
| 1409 | 4409 | 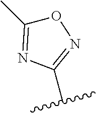 [SMILES: *c1noc(C)n1] | H | 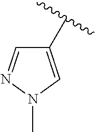 [SMILES: *c1cnn(C)c1] | H | H |
| 1410 | 4410 | 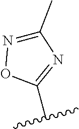 [SMILES: *c1nc(C)no1] | CH₃ | 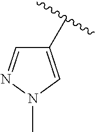 [SMILES: *c1cnn(C)c1] | H | H |
| 1411 | 4411 | 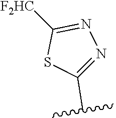 [SMILES: *c1nnc(C(F)F)s1] | CH₃ | 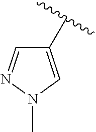 [SMILES: *c1cnn(C)c1] | H | H |
| 1412 | 4412 | 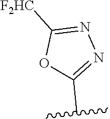 [SMILES: *c1nnc(C(F)F)o1] | CH₃ | 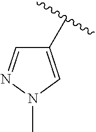 [SMILES: *c1cnn(C)c1] | H | H |
| 1413 | 4413 | 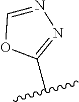 [SMILES: *c1nnco1] | CH₃ | 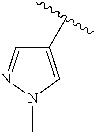 [SMILES: *c1cnn(C)c1] | H | H |
| 1414 | 4414 | 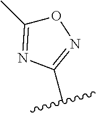 [SMILES: *c1noc(C)n1] | CH₃ | 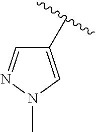 [SMILES: *c1cnn(C)c1] | H | H |
| 1415 | 4415 | 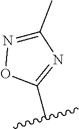 [SMILES: *c1nc(C)no1] | H | 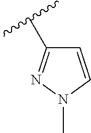 [SMILES: *c1ccn(C)n1] | H | H |
| 1416 | 4416 | 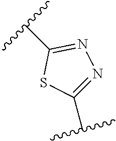 [SMILES: *c1nnc(*)s1] | H | 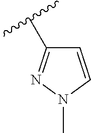 [SMILES: *c1ccn(C)n1] | H | H |
| 1417 | 4417 | 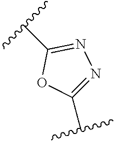 [SMILES: *c1nnc(*)o1] | H | 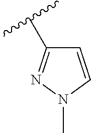 [SMILES: *c1ccn(C)n1] | H | H |
| 1418 | 4418 | 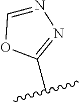 [SMILES: *c1nnco1] | H | 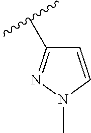 [SMILES: *c1ccn(C)n1] | H | H |
| 1419 | 4419 | 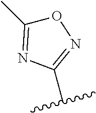 [SMILES: *c1noc(C)n1] | H | 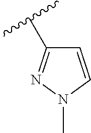 [SMILES: *c1ccn(C)n1] | H | H |
| 1420 | 4420 | 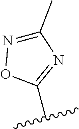 [SMILES: *c1nc(C)no1] | CH₃ | 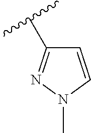 [SMILES: *c1ccn(C)n1] | H | H |
| 1421 | 4421 | 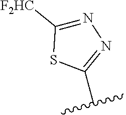 [SMILES: *c1nnc(C(F)F)s1] | CH | 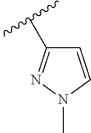 [SMILES: *c1ccn(C)n1] | H | H |
| 1422 | 4422 | 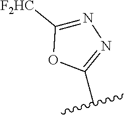 [SMILES: *c1nnc(C(F)F)o1] | CH₃ | 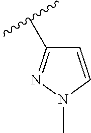 [SMILES: *c1ccn(C)n1] | H | H |
| 1423 | 4423 | 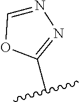 [SMILES: *c1nnco1] | CH₃ | 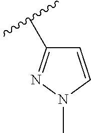 [SMILES: *c1ccn(C)n1] | H | H |
| 1424 | 4424 | 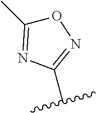 [SMILES: *c1noc(C)n1] | CH₃ | 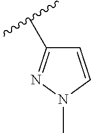 [SMILES: *c1ccn(C)n1] | H | H |
| 1425 | 4425 | 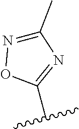 [SMILES: *c1nc(C)no1] | H | 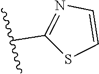 [SMILES: *c1nccs1] | H | H |
| 1426 | 4426 | 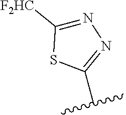 [SMILES: *c1nnc(C(F)F)s1] | H | 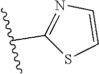 [SMILES: *c1nccs1] | H | H |
| 1427 | 4427 | 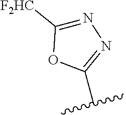 [SMILES: *c1nnc(C(F)F)o1] | H | 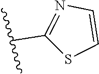 [SMILES: *c1nccs1] | H | H |
| 1428 | 4428 | 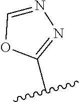 [SMILES: *c1nnco1] | H | 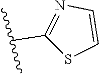 [SMILES: *c1nccs1] | H | H |
| 1429 | 4429 | 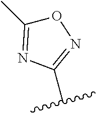 [SMILES: *c1noc(C)n1] | H | 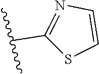 [SMILES: *c1nccs1] | H | H |
| 1430 | 4430 | 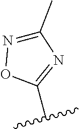 [SMILES: *c1nc(C)no1] | CH | 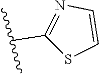 [SMILES: *c1nccs1] | H | H |
| 1431 | 4431 | 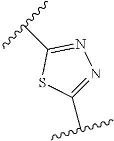 [SMILES: *c1nnc(*)s1] | CH₃ | 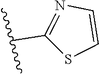 [SMILES: *c1nccs1] | H | H |
| 1432 | 4432 | 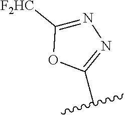 [SMILES: *c1nnc(C(F)F)o1] | CH₃ | 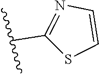 [SMILES: *c1nccs1] | H | H |
| 1433 | 4433 | 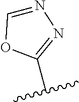 [SMILES: *c1nnco1] | CH₃ | 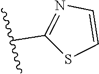 [SMILES: *c1nccs1] | H | H |
| 1434 | 4434 | 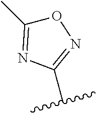 [SMILES: *c1noc(C)n1] | CH₃ | 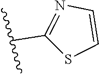 [SMILES: *c1nccs1] | H | H |
| 1435 | 4435 | 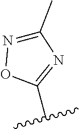 [SMILES: *c1nc(C)no1] | H | 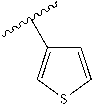 [SMILES: *c1ccsc1] | H | H |
| 1436 | 4436 | 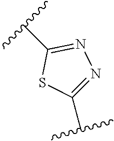 [SMILES: *c1nnc(*)s1] | H | 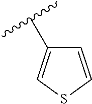 [SMILES: *c1ccsc1] | H | H |
| 1437 | 4437 | 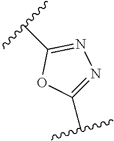 [SMILES: *c1nnc(*)o1] | H | 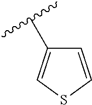 [SMILES: *c1ccsc1] | H | H |
| 1438 | 4438 | 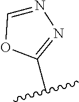 [SMILES: *c1nnco1] | H | 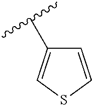 [SMILES: *c1ccsc1] | H | H |
| 1439 | 4439 | 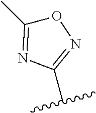 [SMILES: *c1noc(C)n1] | H | 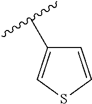 [SMILES: *c1ccsc1] | H | H |
| 1440 | 4440 | 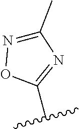 [SMILES: *c1nc(C)no1] | CH₃ | 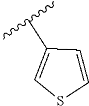 [SMILES: *c1ccsc1] | H | H |
| 1441 | 4441 | 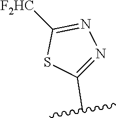 [SMILES: *c1nnc(C(F)F)s1] | CH₃ | 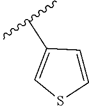 [SMILES: *c1ccsc1] | H | H |
| 1442 | 4442 | 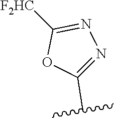 [SMILES: *c1nnc(C(F)F)o1] | CH₃ | 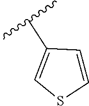 [SMILES: *c1ccsc1] | H | H |
| 1443 | 4443 | 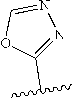 [SMILES: *c1nnco1] | CH₃ | 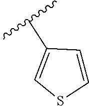 [SMILES: *c1ccsc1] | H | H |
| 1444 | 4444 | 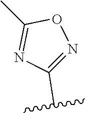 [SMILES: *c1noc(C)n1] | CH₃ | 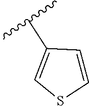 [SMILES: *c1ccsc1] | H | H |
| 1445 | 4445 | 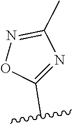 [SMILES: *c1nc(C)no1] | H | 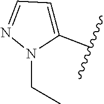 [SMILES: *c1ccnn1CC] | H | F |
| 1446 | 4446 | 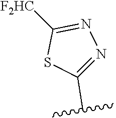 [SMILES: *c1nnc(C(F)F)s1] | H | 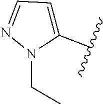 [SMILES: *c1ccnn1CC] | H | F |
| 1447 | 4447 | 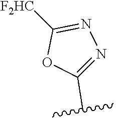 [SMILES: *c1nnc(C(F)F)o1] | H | 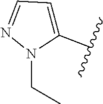 [SMILES: *c1ccnn1CC] | H | F |
| 1448 | 4448 | 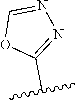 [SMILES: *c1nnco1] | H | 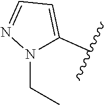 [SMILES: *c1ccnn1CC] | H | F |
| 1449 | 4449 | 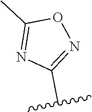 [SMILES: *c1noc(C)n1] | H | 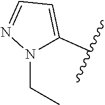 [SMILES: *c1ccnn1CC] | H | F |
| 1450 | 4450 | 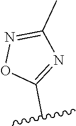 [SMILES: *c1nc(C)no1] | CH₃ | 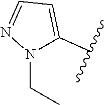 [SMILES: *c1ccnn1CC] | H | F |
| 1451 | 4451 | 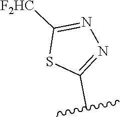 [SMILES: *c1nnc(C(F)F)s1] | CH₃ | 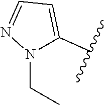 [SMILES: *c1ccnn1CC] | H | F |
| 1452 | 4452 | 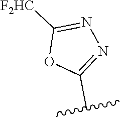 [SMILES: *c1nnc(C(F)F)o1] | CH₃ | 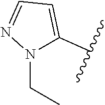 [SMILES: *c1ccnn1CC] | H | F |
| 1453 | 4453 | 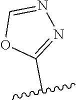 [SMILES: *c1nnco1] | CH₃ | 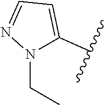 [SMILES: *c1ccnn1CC] | H | F |
| 1454 | 4454 | 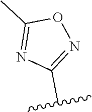 [SMILES: *c1noc(C)n1] | CH₃ | 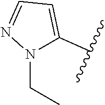 [SMILES: *c1ccnn1CC] | H | F |
| 1455 | 4455 | 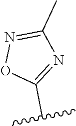 [SMILES: *c1nc(C)no1] | H | 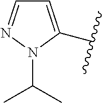 [SMILES: *c1ccnn1C(C)C] | H | F |
| 1456 | 4456 | 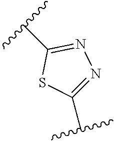 [SMILES: *c1nnc(*)s1] | H | 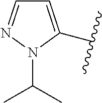 [SMILES: *c1ccnn1C(C)C] | H | F |
| 1457 | 4457 | 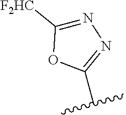 [SMILES: *c1nnc(C(F)F)o1] | H | 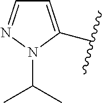 [SMILES: *c1ccnn1C(C)C] | H | F |
| 1458 | 4458 | 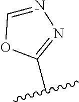 [SMILES: *c1nnco1] | H | 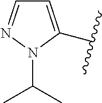 [SMILES: *c1ccnn1C(C)C] | H | F |
| 1459 | 4459 | 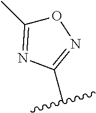 [SMILES: *c1noc(C)n1] | H | 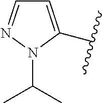 [SMILES: *c1ccnn1C(C)C] | H | F |
| 1460 | 4460 | 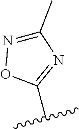 [SMILES: *c1nc(C)no1] | CH₃ | 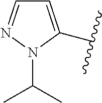 [SMILES: *c1ccnn1C(C)C] | H | F |
| 1461 | 4461 | 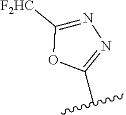 [SMILES: *c1nnc(C(F)F)o1] | CH₃ | 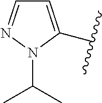 [SMILES: *c1ccnn1C(C)C] | H | F |
| 1462 | 4462 | 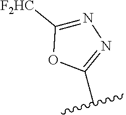 [SMILES: *c1nnc(C(F)F)o1] | CH₃ | 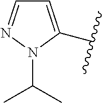 [SMILES: *c1ccnn1C(C)C] | H | F |
| 1463 | 4463 | 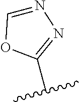 [SMILES: *c1nnco1] | CH₃ | 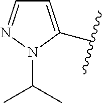 [SMILES: *c1ccnn1C(C)C] | H | F |
| 1464 | 4464 | 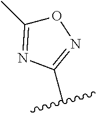 [SMILES: *c1noc(C)n1] | CH₃ | 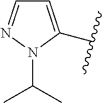 [SMILES: *c1ccnn1C(C)C] | H | F |
| 1465 | 4465 | 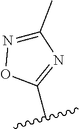 [SMILES: *c1nc(C)no1] | H | 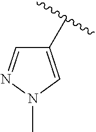 [SMILES: *c1cnn(C)c1] | H | F |
| 1466 | 4466 | 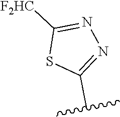 [SMILES: *c1nnc(C(F)F)s1] | H | 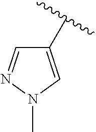 [SMILES: *c1cnn(C)c1] | H | F |
| 1467 | 4467 | 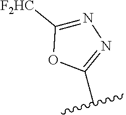 [SMILES: *c1nnc(C(F)F)o1] | H | 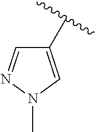 [SMILES: *c1cnn(C)c1] | H | F |
| 1468 | 4468 | 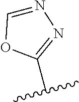 [SMILES: *c1nnco1] | H | 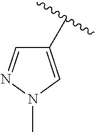 [SMILES: *c1cnn(C)c1] | H | F |
| 1469 | 4469 | 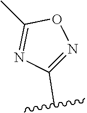 [SMILES: *c1noc(C)n1] | H | 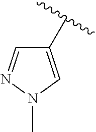 [SMILES: *c1cnn(C)c1] | H | F |
| 1470 | 4470 | 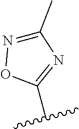 [SMILES: *c1nc(C)no1] | CH₃ | 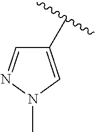 [SMILES: *c1cnn(C)c1] | H | F |
| 1471 | 4471 | 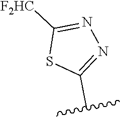 [SMILES: *c1nnc(C(F)F)s1] | CH₃ | 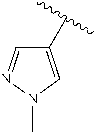 [SMILES: *c1cnn(C)c1] | H | F |
| 1472 | 4472 | 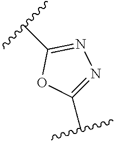 [SMILES: *c1nnc(*)o1] | CH₃ | 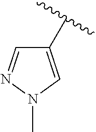 [SMILES: *c1cnn(C)c1] | H | F |
| 1473 | 4473 | 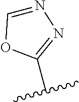 [SMILES: *c1nnco1] | CH₃ | 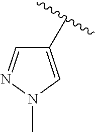 [SMILES: *c1cnn(C)c1] | H | F |
| 1474 | 4474 | 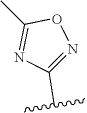 [SMILES: *c1noc(C)n1] | CH₃ | 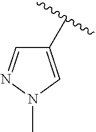 [SMILES: *c1cnn(C)c1] | H | F |
| 1475 | 4475 | 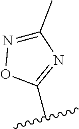 [SMILES: *c1nc(C)no1] | H | 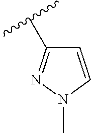 [SMILES: *c1ccn(C)n1] | H | F |
| 1476 | 4476 | 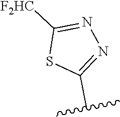 [SMILES: *c1nnc(C(F)F)s1] | H | 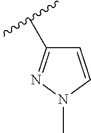 [SMILES: *c1ccn(C)n1] | H | F |
| 1477 | 4477 | 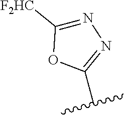 [SMILES: *c1nnc(C(F)F)o1] | H | 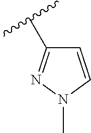 [SMILES: *c1ccn(C)n1] | H | F |
| 1478 | 4478 | 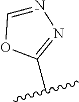 [SMILES: *c1nnco1] | H | 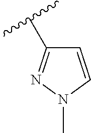 [SMILES: *c1ccn(C)n1] | H | F |
| 1479 | 4479 | 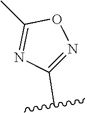 [SMILES: *c1noc(C)n1] | H | 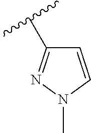 [SMILES: *c1ccn(C)n1] | H | F |
| 1480 | 4480 | 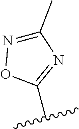 [SMILES: *c1nc(C)no1] | CH₃ | 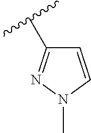 [SMILES: *c1ccn(C)n1] | H | F |
| 1481 | 4481 | 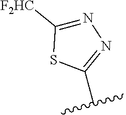 [SMILES: *c1nnc(C(F)F)s1] | CH₃ | 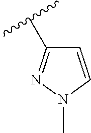 [SMILES: *c1ccn(C)n1] | H | F |
| 1482 | 4482 | 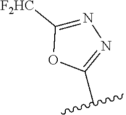 [SMILES: *c1nnc(C(F)F)o1] | CH₃ | 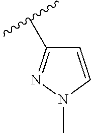 [SMILES: *c1ccn(C)n1] | H | F |
| 1483 | 4483 | 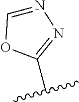 [SMILES: *c1nnco1] | CH₃ | 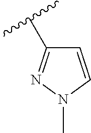 [SMILES: *c1ccn(C)n1] | H | F |
| 1484 | 4484 | 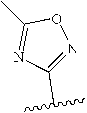 [SMILES: *c1noc(C)n1] | CH₃ | 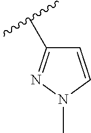 [SMILES: *c1ccn(C)n1] | H | F |
| 1485 | 4485 | 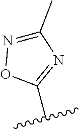 [SMILES: *c1nc(C)no1] | H | 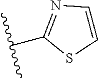 [SMILES: *c1nccs1] | H | F |
| 1486 | 4486 | 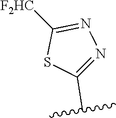 [SMILES: *c1nnc(C(F)F)s1] | H | 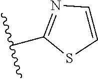 [SMILES: *c1nccs1] | H | F |
| 1487 | 4487 | 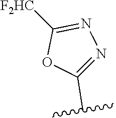 [SMILES: *c1nnc(C(F)F)o1] | H | 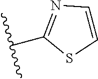 [SMILES: *c1nccs1] | H | F |
| 1488 | 4488 | 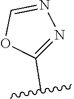 [SMILES: *c1nnco1] | H | 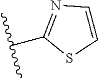 [SMILES: *c1nccs1] | H | F |
| 1489 | 4489 | 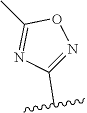 [SMILES: *c1noc(C)n1] | H | 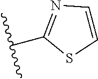 [SMILES: *c1nccs1] | H | F |
| 1490 | 4490 | 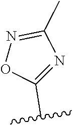 [SMILES: *c1nc(C)no1] | CH₃ | 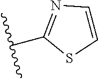 [SMILES: *c1nccs1] | H | F |
| 1491 | 4491 | 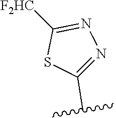 [SMILES: *c1nnc(C(F)F)s1] | CH₃ | 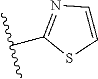 [SMILES: *c1nccs1] | H | F |
| 1492 | 4492 | 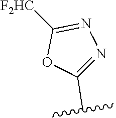 [SMILES: *c1nnc(C(F)F)o1] | CH₃ | 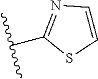 [SMILES: *c1nccs1] | H | F |
| 1493 | 4493 | 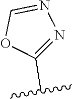 [SMILES: *c1nnco1] | CH₃ | 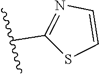 [SMILES: *c1nccs1] | H | F |
| 1494 | 4494 | 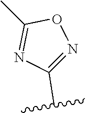 [SMILES: *c1noc(C)n1] | CH₃ | 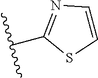 [SMILES: *c1nccs1] | H | F |
| 1495 | 4495 | 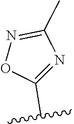 [SMILES: *c1nc(C)no1] | H | 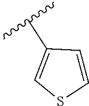 [SMILES: *c1ccsc1] | H | F |
| 1496 | 4496 | 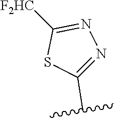 [SMILES: *c1nnc(C(F)F)s1] | H | 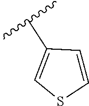 [SMILES: *c1ccsc1] | H | F |
| 1497 | 4497 | 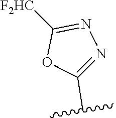 [SMILES: *c1nnc(C(F)F)o1] | H | 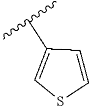 [SMILES: *c1ccsc1] | H | F |
| 1498 | 4498 | 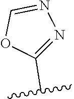 [SMILES: *c1nnco1] | H | 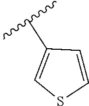 [SMILES: *c1ccsc1] | H | F |
| 1499 | 4499 | 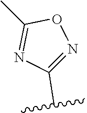 [SMILES: *c1noc(C)n1] | H | 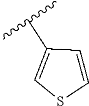 [SMILES: *c1ccsc1] | H | F |
| 1500 | 4500 | 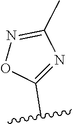 [SMILES: *c1nc(C)no1] | CH₃ | 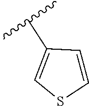 [SMILES: *c1ccsc1] | H | F |
| 1501 | 4501 | 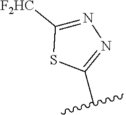 [SMILES: *c1nnc(C(F)F)s1] | CH₃ | 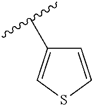 [SMILES: *c1ccsc1] | H | F |
| 1502 | 4502 | 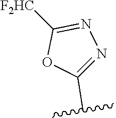 [SMILES: *c1nnc(C(F)F)o1] | CH₃ | 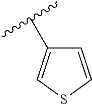 [SMILES: *c1ccsc1] | H | F |
| 1503 | 4503 | 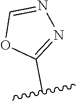 [SMILES: *c1nnco1] | CH₃ | 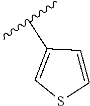 [SMILES: *c1ccsc1] | H | F |
| 1504 | 4504 | 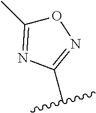 [SMILES: *c1noc(C)n1] | CH₃ | 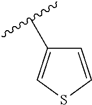 [SMILES: *c1ccsc1] | H | F |
| 1505 | 4505 | 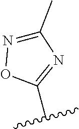 [SMILES: *c1nc(C)no1] | H | 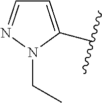 [SMILES: *c1ccnn1CC] | F | H |
| 1506 | 4506 | 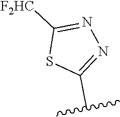 [SMILES: *c1nnc(C(F)F)s1] | H | 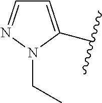 [SMILES: *c1ccnn1CC] | F | H |
| 1507 | 4507 | 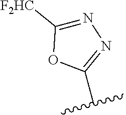 [SMILES: *c1nnc(C(F)F)o1] | H | 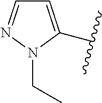 [SMILES: *c1ccnn1CC] | F | H |
| 1508 | 4508 | 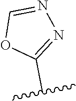 [SMILES: *c1nnco1] | H | 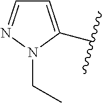 [SMILES: *c1ccnn1CC] | F | H |
| 1509 | 4509 | 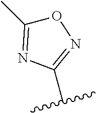 [SMILES: *c1noc(C)n1] | H | 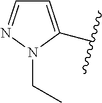 [SMILES: *c1ccnn1CC] | F | H |
| 1510 | 4510 | 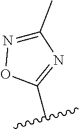 [SMILES: *c1nc(C)no1] | CH₃ | 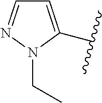 [SMILES: *c1ccnn1CC] | F | H |
| 1511 | 4511 | 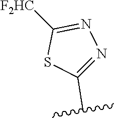 [SMILES: *c1nnc(C(F)F)s1] | CH₃ | 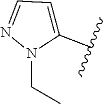 [SMILES: *c1ccnn1CC] | F | H |
| 1512 | 4512 | 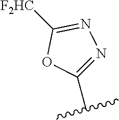 [SMILES: *c1nnc(C(F)F)o1] | CH₃ | 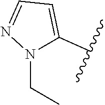 [SMILES: *c1ccnn1CC] | F | H |
| 1513 | 4513 | 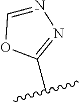 [SMILES: *c1nnco1] | CH₃ | 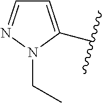 [SMILES: *c1ccnn1CC] | F | H |
| 1514 | 4514 | 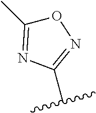 [SMILES: *c1noc(C)n1] | CH₃ | 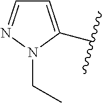 [SMILES: *c1ccnn1CC] | F | H |
| 1515 | 4515 | 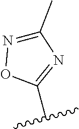 [SMILES: *c1nc(C)no1] | H | 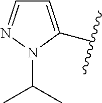 [SMILES: *c1ccnn1C(C)C] | F | H |
| 1516 | 4516 | 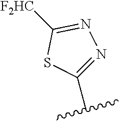 [SMILES: *c1nnc(C(F)F)s1] | H | 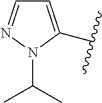 [SMILES: *c1ccnn1C(C)C] | F | H |
| 1517 | 4517 | 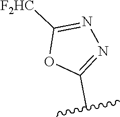 [SMILES: *c1nnc(C(F)F)o1] | H | 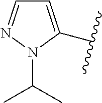 [SMILES: *c1ccnn1C(C)C] | F | H |
| 1518 | 4518 | 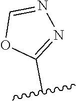 [SMILES: *c1nnco1] | H | 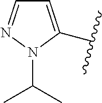 [SMILES: *c1ccnn1C(C)C] | F | H |
| 1519 | 4519 | 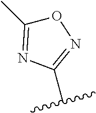 [SMILES: *c1noc(C)n1] | H | 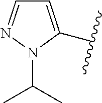 [SMILES: *c1ccnn1C(C)C] | F | H |
| 1520 | 4520 | 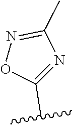 [SMILES: *c1nc(C)no1] | CH₃ | 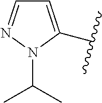 [SMILES: *c1ccnn1C(C)C] | F | H |
| 1521 | 4521 | 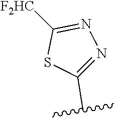 [SMILES: *c1nnc(C(F)F)s1] | CH₃ | 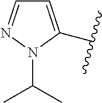 [SMILES: *c1ccnn1C(C)C] | F | H |
| 1522 | 4522 | 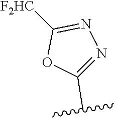 [SMILES: *c1nnc(C(F)F)o1] | CH₃ | 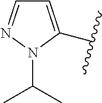 [SMILES: *c1ccnn1C(C)C] | F | H |
| 1523 | 4523 | 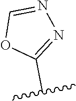 [SMILES: *c1nnco1] | CH₃ | 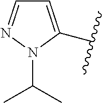 [SMILES: *c1ccnn1C(C)C] | F | H |
| 1524 | 4524 | 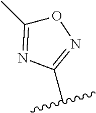 [SMILES: *c1noc(C)n1] | CH₃ | 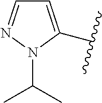 [SMILES: *c1ccnn1C(C)C] | F | H |
| 1525 | 4525 | 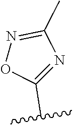 [SMILES: *c1nc(C)no1] | H | 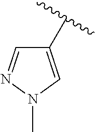 [SMILES: *c1cnn(C)c1] | F | H |
| 1526 | 4526 | 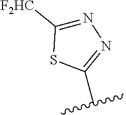 [SMILES: *c1nnc(C(F)F)s1] | H | 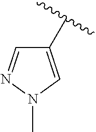 [SMILES: *c1cnn(C)c1] | F | H |
| 1527 | 4527 | 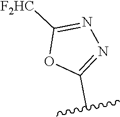 [SMILES: *c1nnc(C(F)F)o1] | H | 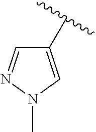 [SMILES: *c1cnn(C)c1] | F | H |
| 1528 | 4528 | 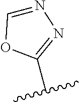 [SMILES: *c1nnco1] | H | 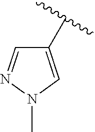 [SMILES: *c1cnn(C)c1] | F | H |
| 1529 | 4529 | 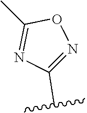 [SMILES: *c1noc(C)n1] | H | 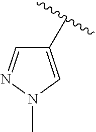 [SMILES: *c1cnn(C)c1] | F | H |
| 1530 | 4530 | 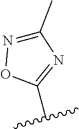 [SMILES: *c1nc(C)no1] | CH₃ | 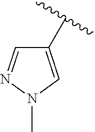 [SMILES: *c1cnn(C)c1] | F | H |
| 1531 | 4531 | 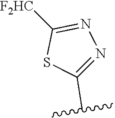 [SMILES: *c1nnc(C(F)F)s1] | CH₃ | 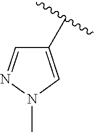 [SMILES: *c1cnn(C)c1] | F | H |
| 1532 | 4532 | 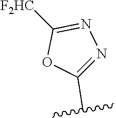 [SMILES: *c1nnc(C(F)F)o1] | CH₃ | 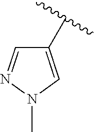 [SMILES: *c1cnn(C)c1] | F | H |
| 1533 | 4533 | 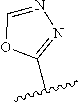 [SMILES: *c1nnco1] | CH₃ | 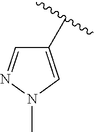 [SMILES: *c1cnn(C)c1] | F | H |
| 1534 | 4534 | 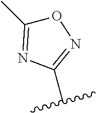 [SMILES: *c1noc(C)n1] | CH₃ | 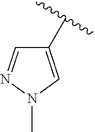 [SMILES: *c1cnn(C)c1] | F | H |
| 1535 | 4535 | 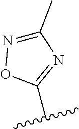 [SMILES: *c1nc(C)no1] | H | 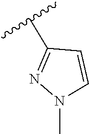 [SMILES: *c1ccn(C)n1] | F | H |
| 1536 | 4536 | 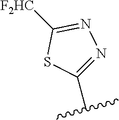 [SMILES: *c1nnc(C(F)F)s1] | H | 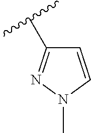 [SMILES: *c1ccn(C)n1] | F | H |
| 1537 | 4537 | 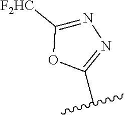 [SMILES: *c1nnc(C(F)F)o1] | H | 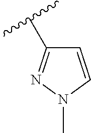 [SMILES: *c1ccn(C)n1] | F | H |
| 1538 | 4538 | 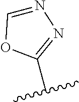 [SMILES: *c1nnco1] | H | 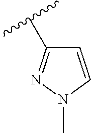 [SMILES: *c1ccn(C)n1] | F | H |
| 1539 | 4539 | 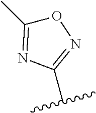 [SMILES: *c1noc(C)n1] | H | 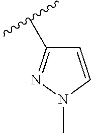 [SMILES: *c1ccn(C)n1] | F | H |
| 1540 | 4540 | 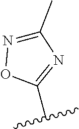 [SMILES: *c1nc(C)no1] | CH₃ | 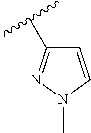 [SMILES: *c1ccn(C)n1] | F | H |
| 1541 | 4541 | 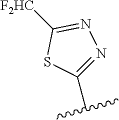 [SMILES: *c1nnc(C(F)F)s1] | CH₃ | 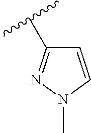 [SMILES: *c1ccn(C)n1] | F | H |
| 1542 | 4542 | 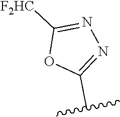 [SMILES: *c1nnc(C(F)F)o1] | CH₃ | 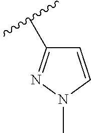 [SMILES: *c1ccn(C)n1] | F | H |
| 1543 | 4543 | 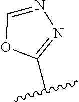 [SMILES: *c1nnco1] | CH₃ | 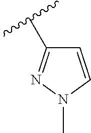 [SMILES: *c1ccn(C)n1] | F | H |
| 1544 | 4544 | 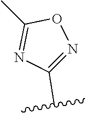 [SMILES: *c1noc(C)n1] | CH₃ | 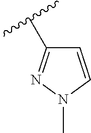 [SMILES: *c1ccn(C)n1] | F | H |
| 1545 | 4545 | 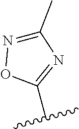 [SMILES: *c1nc(C)no1] | H | 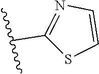 [SMILES: *c1nccs1] | F | H |
| 1546 | 4546 | 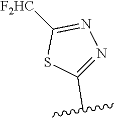 [SMILES: *c1nnc(C(F)F)s1] | H | 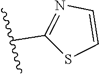 [SMILES: *c1nccs1] | F | H |
| 1547 | 4547 | 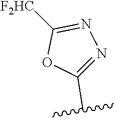 [SMILES: *c1nnc(C(F)F)o1] | H | 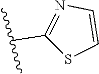 [SMILES: *c1nccs1] | F | H |
| 1548 | 4548 | 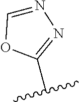 [SMILES: *c1nnco1] | H | 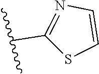 [SMILES: *c1nccs1] | F | H |
| 1549 | 4549 | 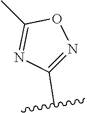 [SMILES: *c1noc(C)n1] | H | 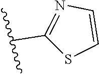 [SMILES: *c1nccs1] | F | H |
| 1550 | 4550 | 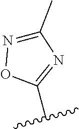 [SMILES: *c1nc(C)no1] | CH₃ | 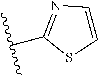 [SMILES: *c1nccs1] | F | H |
| 1551 | 4551 | 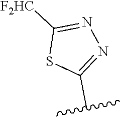 [SMILES: *c1nnc(C(F)F)s1] | CH | 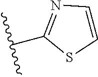 [SMILES: *c1nccs1] | F | H |
| 1552 | 4552 | 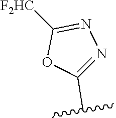 [SMILES: *c1nnc(C(F)F)o1] | CH₃ | 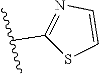 [SMILES: *c1nccs1] | F | H |
| 1553 | 4553 | 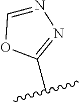 [SMILES: *c1nnco1] | CH₃ | 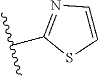 [SMILES: *c1nccs1] | F | H |
| 1554 | 4554 | 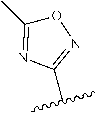 [SMILES: *c1noc(C)n1] | CH₃ | 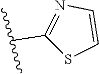 [SMILES: *c1nccs1] | F | H |
| 1555 | 4555 | 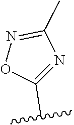 [SMILES: *c1nc(C)no1] | H | 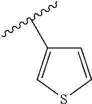 [SMILES: *c1ccsc1] | F | H |
| 1556 | 4556 | 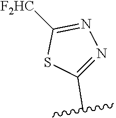 [SMILES: *c1nnc(C(F)F)s1] | H | 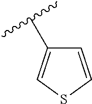 [SMILES: *c1ccsc1] | F | H |
| 1557 | 4557 | 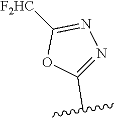 [SMILES: *c1nnc(C(F)F)o1] | H | 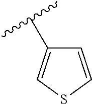 [SMILES: *c1ccsc1] | F | H |
| 1558 | 4558 | 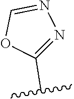 [SMILES: *c1nnco1] | H | 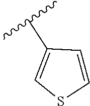 [SMILES: *c1ccsc1] | F | H |
| 1559 | 4559 | 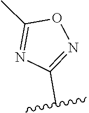 [SMILES: *c1noc(C)n1] | H | 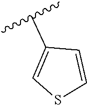 [SMILES: *c1ccsc1] | F | H |
| 1560 | 4560 | 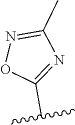 [SMILES: *c1nc(C)no1] | CH₃ | 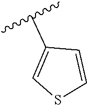 [SMILES: *c1ccsc1] | F | H |
| 1561 | 4561 | 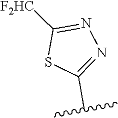 [SMILES: *c1nnc(C(F)F)s1] | CH₃ | 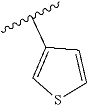 [SMILES: *c1ccsc1] | F | H |
| 1562 | 4562 | 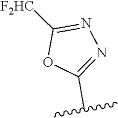 [SMILES: *c1nnc(C(F)F)o1] | CH₃ | 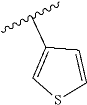 [SMILES: *c1ccsc1] | F | H |
| 1563 | 4563 | 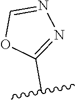 [SMILES: *c1nnco1] | CH₃ | 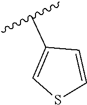 [SMILES: *c1ccsc1] | F | H |
| 1564 | 4564 | 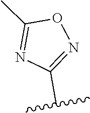 [SMILES: *c1noc(C)n1] | CH₃ | 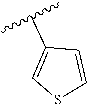 [SMILES: *c1ccsc1] | F | H |
| 1565 | 4565 | 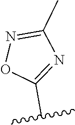 [SMILES: *c1nc(C)no1] | H | 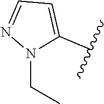 [SMILES: *c1ccnn1CC] | H | Cl |
| 1566 | 4566 | 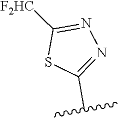 [SMILES: *c1nnc(C(F)F)s1] | H | 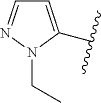 [SMILES: *c1ccnn1CC] | H | Cl |
| 1567 | 4567 | 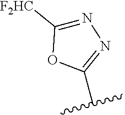 [SMILES: *c1nnc(C(F)F)o1] | H | 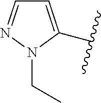 [SMILES: *c1ccnn1CC] | H | Cl |
| 1568 | 4568 | 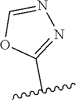 [SMILES: *c1nnco1] | H | 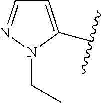 [SMILES: *c1ccnn1CC] | H | Cl |
| 1569 | 4569 | 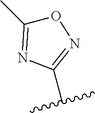 [SMILES: *c1noc(C)n1] | H | 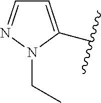 [SMILES: *c1ccnn1CC] | H | Cl |
| 1570 | 4570 | 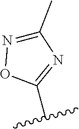 [SMILES: *c1nc(C)no1] | CH₃ | 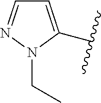 [SMILES: *c1ccnn1CC] | H | Cl |
| 1571 | 4571 | 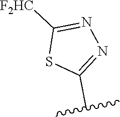 [SMILES: *c1nnc(C(F)F)s1] | CH₃ | 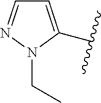 [SMILES: *c1ccnn1CC] | H | Cl |
| 1572 | 4572 | 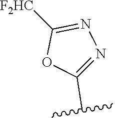 [SMILES: *c1nnc(C(F)F)o1] | CH₃ | 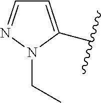 [SMILES: *c1ccnn1CC] | H | Cl |
| 1573 | 4573 | 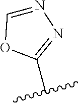 [SMILES: *c1nnco1] | CH₃ | 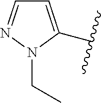 [SMILES: *c1ccnn1CC] | H | Cl |
| 1574 | 4574 | 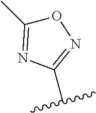 [SMILES: *c1noc(C)n1] | CH₃ | 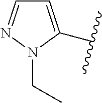 [SMILES: *c1ccnn1CC] | H | Cl |
| 1575 | 4575 | 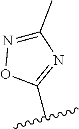 [SMILES: *c1nc(C)no1] | H | 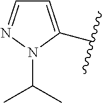 [SMILES: *c1ccnn1C(C)C] | H | Cl |
| 1576 | 4576 | 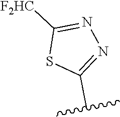 [SMILES: *c1nnc(C(F)F)s1] | H | 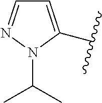 [SMILES: *c1ccnn1C(C)C] | H | Cl |
| 1577 | 4577 | 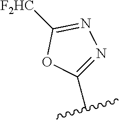 [SMILES: *c1nnc(C(F)F)o1] | H | 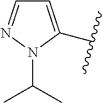 [SMILES: *c1ccnn1C(C)C] | H | Cl |
| 1578 | 4578 | 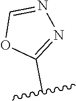 [SMILES: *c1nnco1] | H | 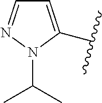 [SMILES: *c1ccnn1C(C)C] | H | Cl |
| 1579 | 4579 | 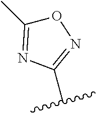 [SMILES: *c1noc(C)n1] | H | 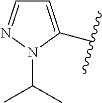 [SMILES: *c1ccnn1C(C)C] | H | Cl |
| 1580 | 4580 | 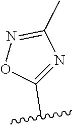 [SMILES: *c1nc(C)no1] | CH₃ | 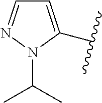 [SMILES: *c1ccnn1C(C)C] | H | Cl |
| 1581 | 4581 | 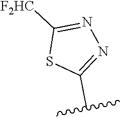 [SMILES: *c1nnc(C(F)F)s1] | CH₃ | 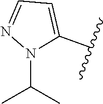 [SMILES: *c1ccnn1C(C)C] | H | Cl |
| 1582 | 4582 | 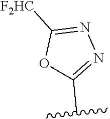 [SMILES: *c1nnc(C(F)F)o1] | CH₃ | 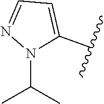 [SMILES: *c1ccnn1C(C)C] | H | Cl |
| 1583 | 4583 | 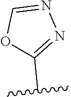 [SMILES: *c1nnco1] | CH₃ | 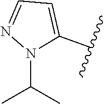 [SMILES: *c1ccnn1C(C)C] | H | Cl |
| 1584 | 4584 | 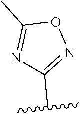 [SMILES: *c1noc(C)n1] | CH₃ | 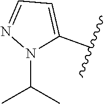 [SMILES: *c1ccnn1C(C)C] | I | Cl |
| 1585 | 4585 | 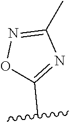 [SMILES: *c1nc(C)no1] | H | 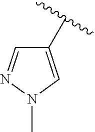 [SMILES: *c1cnn(C)c1] | H | E |
| 1586 | 4586 | 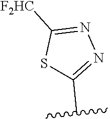 [SMILES: *c1nnc(C(F)F)s1] | H | 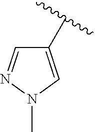 [SMILES: *c1cnn(C)c1] | H | Cl |
| 1587 | 4587 | 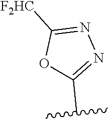 [SMILES: *c1nnc(C(F)F)o1] | H | 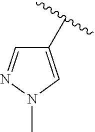 [SMILES: *c1cnn(C)c1] | H | Cl |
| 1588 | 4588 | 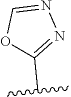 [SMILES: *c1nnco1] | H | 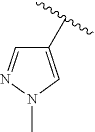 [SMILES: *c1cnn(C)c1] | H | Cl |
| 1589 | 4589 | 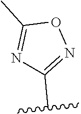 [SMILES: *c1noc(C)n1] | H | 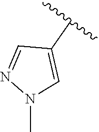 [SMILES: *c1cnn(C)c1] | H | Cl |
| 1590 | 4590 | 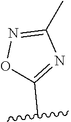 [SMILES: *c1nc(C)no1] | CH₃ | 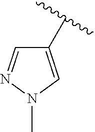 [SMILES: *c1cnn(C)c1] | H | Cl |
| 1591 | 4591 | 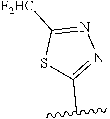 [SMILES: *c1nnc(C(F)F)s1] | CH₃ | 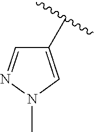 [SMILES: *c1cnn(C)c1] | H | Cl |
| 1592 | 4592 | 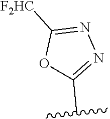 [SMILES: *c1nnc(C(F)F)o1] | CH₃ | 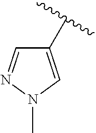 [SMILES: *c1cnn(C)c1] | H | Cl |
| 1593 | 4593 | 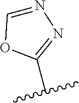 [SMILES: *c1nnco1] | CH₃ | 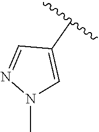 [SMILES: *c1cnn(C)c1] | H | Cl |
| 1594 | 4594 | 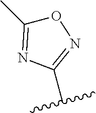 [SMILES: *c1noc(C)n1] | CH₃ | 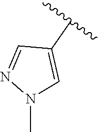 [SMILES: *c1cnn(C)c1] | H | Cl |
| 1595 | 4595 | 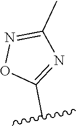 [SMILES: *c1nc(C)no1] | H | 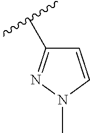 [SMILES: *c1ccn(C)n1] | H | Cl |
| 1596 | 4596 | 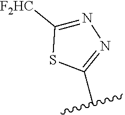 [SMILES: *c1nnc(C(F)F)s1] | H | 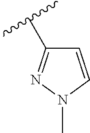 [SMILES: *c1ccn(C)n1] | H | Cl |
| 1597 | 4597 | 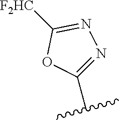 [SMILES: *c1nnc(C(F)F)o1] | H | 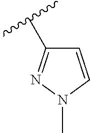 [SMILES: *c1ccn(C)n1] | H | Cl |
| 1598 | 4598 | 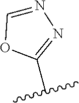 [SMILES: *c1nnco1] | H | 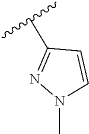 [SMILES: *c1ccn(C)n1] | H | Cl |
| 1599 | 4599 | 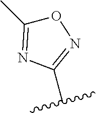 [SMILES: *c1noc(C)n1] | H | 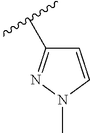 [SMILES: *c1ccn(C)n1] | H | Cl |
| 1600 | 4600 | 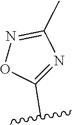 [SMILES: *c1nc(C)no1] | CH₃ | 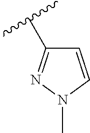 [SMILES: *c1ccn(C)n1] | H | Cl |
| 1601 | 4601 | 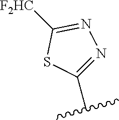 [SMILES: *c1nnc(C(F)F)s1] | CH₃ | 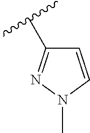 [SMILES: *c1ccn(C)n1] | H | Cl |
| 1602 | 4602 | 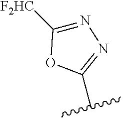 [SMILES: *c1nnc(C(F)F)o1] | CH₃ | 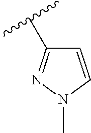 [SMILES: *c1ccn(C)n1] | H | Cl |
| 1603 | 4603 | 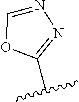 [SMILES: *c1nnco1] | CH₃ | 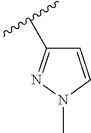 [SMILES: *c1ccn(C)n1] | H | Cl |
| 1604 | 4604 | 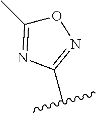 [SMILES: *c1noc(C)n1] | CH₃ | 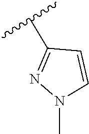 [SMILES: *c1ccn(C)n1] | H | Cl |
| 1605 | 4605 | 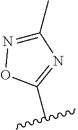 [SMILES: *c1nc(C)no1] | H | 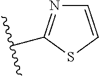 [SMILES: *c1nccs1] | H | Cl |
| 1606 | 4606 | 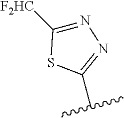 [SMILES: *c1nnc(C(F)F)s1] | H | 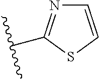 [SMILES: *c1nccs1] | H | Cl |
| 1607 | 4607 | 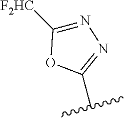 [SMILES: *c1nnc(C(F)F)o1] | H | 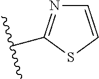 [SMILES: *c1nccs1] | H | Cl |
| 1608 | 4608 | 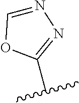 [SMILES: *c1nnco1] | H | 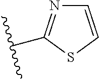 [SMILES: *c1nccs1] | H | Cl |
| 1609 | 4609 | 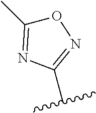 [SMILES: *c1noc(C)n1] | H | 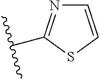 [SMILES: *c1nccs1] | H | Cl |
| 1610 | 4610 | 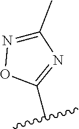 [SMILES: *c1nc(C)no1] | CH₃ | 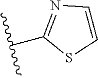 [SMILES: *c1nccs1] | H | Cl |
| 1611 | 4611 | 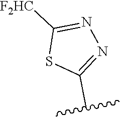 [SMILES: *c1nnc(C(F)F)s1] | CH₃ | 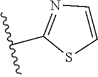 [SMILES: *c1nccs1] | H | Cl |
| 1612 | 4612 | 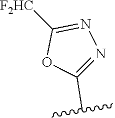 [SMILES: *c1nnc(C(F)F)o1] | CH₃ | 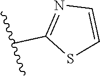 [SMILES: *c1nccs1] | H | Cl |
| 1613 | 4613 | 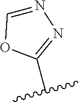 [SMILES: *c1nnco1] | CH₃ | 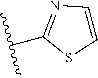 [SMILES: *c1nccs1] | H | Cl |
| 1614 | 4614 | 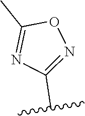 [SMILES: *c1noc(C)n1] | CH₃ | 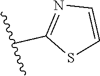 [SMILES: *c1nccs1] | H | Cl |
| 1615 | 4615 | 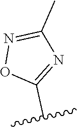 [SMILES: *c1nc(C)no1] | H | 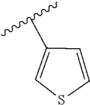 [SMILES: *c1ccsc1] | H | Cl |
| 1616 | 4616 | 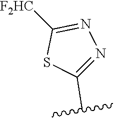 [SMILES: *c1nnc(C(F)F)s1] | H | 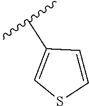 [SMILES: *c1ccsc1] | H | Cl |
| 1617 | 4617 | 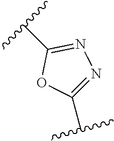 [SMILES: *c1nnc(*)o1] | H | 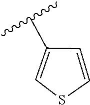 [SMILES: *c1ccsc1] | H | Cl |
| 1618 | 4618 | 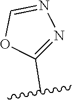 [SMILES: *c1nnco1] | H | 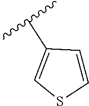 [SMILES: *c1ccsc1] | H | E |
| 1619 | 4619 | 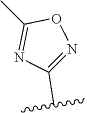 [SMILES: *c1noc(C)n1] | H | 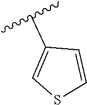 [SMILES: *c1ccsc1] | H | Cl |
| 1620 | 4620 | 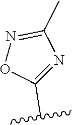 [SMILES: *c1nc(C)no1] | CH₃ | 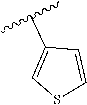 [SMILES: *c1ccsc1] | H | Cl |
| 1621 | 4621 | 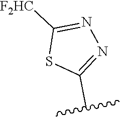 [SMILES: *c1nnc(C(F)F)s1] | CH₃ | 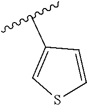 [SMILES: *c1ccsc1] | H | Cl |
| 1622 | 4622 | 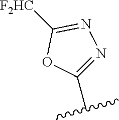 [SMILES: *c1nnc(C(F)F)o1] | CH₃ | 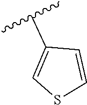 [SMILES: *c1ccsc1] | H | 5 |
| 1623 | 4623 | 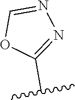 [SMILES: *c1nnco1] | CH₃ | 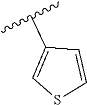 [SMILES: *c1ccsc1] | H | Cl |
| 1624 | 4624 | 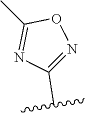 [SMILES: *c1noc(C)n1] | CH₃ | 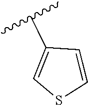 [SMILES: *c1ccsc1] | H | 5 |
| 1625 | 4625 | 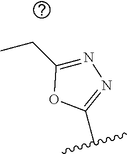 [SMILES: *c1nnc(CC)o1] | CH₃ | 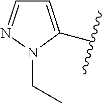 [SMILES: *c1ccnn1CC] | H | H |
| 1626 | 4626 | 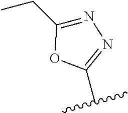 [SMILES: *c1nnc(CC)o1] | CH₃ | 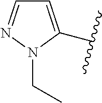 [SMILES: *c1ccnn1CC] | H | F |
| 1627 | 4627 | 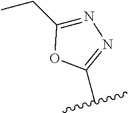 [SMILES: *c1nnc(CC)o1] | CH₃ | 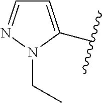 [SMILES: *c1ccnn1CC] | F | H |
| 1628 | 4628 | 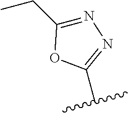 [SMILES: *c1nnc(CC)o1] | CH₃ | 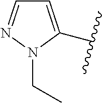 [SMILES: *c1ccnn1CC] | H | Cl |
| 1629 | 4629 | 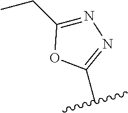 [SMILES: *c1nnc(CC)o1] | H | 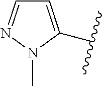 [SMILES: *c1ccnn1C] | H | H |
| 1630 | 4630 | 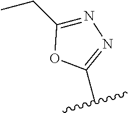 [SMILES: *c1nnc(CC)o1] | H | 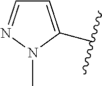 [SMILES: *c1ccnn1C] | H | F |
| 1631 | 4631 | 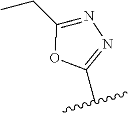 [SMILES: *c1nnc(CC)o1] | H | 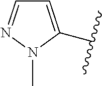 [SMILES: *c1ccnn1C] | F | H |
| 1632 | 4632 | 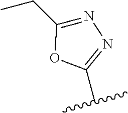 [SMILES: *c1nnc(CC)o1] | H | 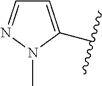 [SMILES: *c1ccnn1C] | H | Cl |
| 1633 | 4633 | 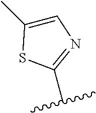 [SMILES: *c1ncc(C)s1] | H | 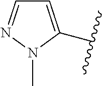 [SMILES: *c1ccnn1C] | H | H |
| 1634 | 4634 | 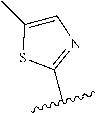 [SMILES: *c1ncc(C)s1] | CH₃ | 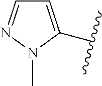 [SMILES: *c1ccnn1C] | H | H |
| 1635 | 4635 | 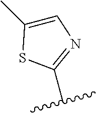 [SMILES: *c1ncc(C)s1] | H | 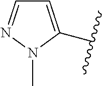 [SMILES: *c1ccnn1C] | H | F |
| 1636 | 4636 | 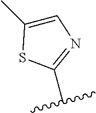 [SMILES: *c1ncc(C)s1] | CH₃ | 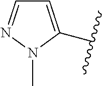 [SMILES: *c1ccnn1C] | H | F |
| 1637 | 4637 | 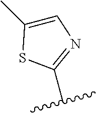 [SMILES: *c1ncc(C)s1] | H | 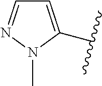 [SMILES: *c1ccnn1C] | F | H |
| 1638 | 4638 | 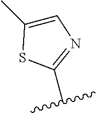 [SMILES: *c1ncc(C)s1] | CH₃ | 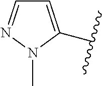 [SMILES: *c1ccnn1C] | F | H |
| 1639 | 4639 | 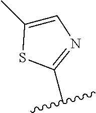 [SMILES: *c1ncc(C)s1] | H | 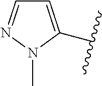 [SMILES: *c1ccnn1C] | H | Cl |
| 1640 | 4640 | 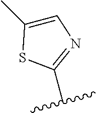 [SMILES: *c1ncc(C)s1] | CH₃ | 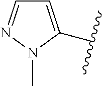 [SMILES: *c1ccnn1C] | H | Cl |
| 1641 | 4641 | 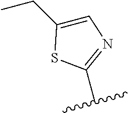 [SMILES: *c1ncc(CC)s1] | H | 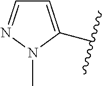 [SMILES: *c1ccnn1C] | H | H |
| 1642 | 4642 | 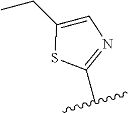 [SMILES: *c1ncc(CC)s1] | CH₃ | 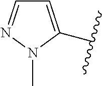 [SMILES: *c1ccnn1C] | H | H |
| 1643 | 4643 | 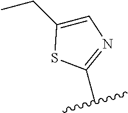 [SMILES: *c1ncc(CC)s1] | H | 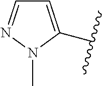 [SMILES: *c1ccnn1C] | H | F |
| 1644 | 4644 | 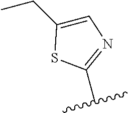 [SMILES: *c1ncc(CC)s1] | CH₃ | 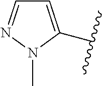 [SMILES: *c1ccnn1C] | H | F |
| 1645 | 4645 | 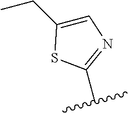 [SMILES: *c1ncc(CC)s1] | H | 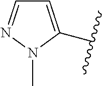 [SMILES: *c1ccnn1C] | F | H |
| 1646 | 4646 | 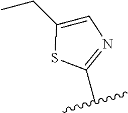 [SMILES: *c1ncc(CC)s1] | CH | 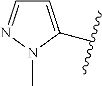 [SMILES: *c1ccnn1C] | F | H |
| 1647 | 4647 | 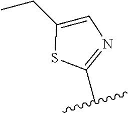 [SMILES: *c1ncc(CC)s1] | H | 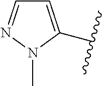 [SMILES: *c1ccnn1C] | H | Cl |
| 1648 | 4648 | 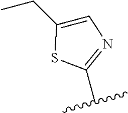 [SMILES: *c1ncc(CC)s1] | CH₃ | 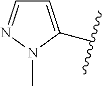 [SMILES: *c1ccnn1C] | H | Cl |
| 1649 | 4649 | 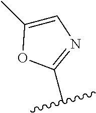 [SMILES: *c1ncc(C)o1] | H | 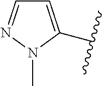 [SMILES: *c1ccnn1C] | H | H |
| 1650 | 4650 | 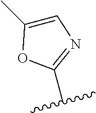 [SMILES: *c1ncc(C)o1] | CH₃ | 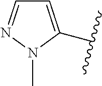 [SMILES: *c1ccnn1C] | H | H |
| 1651 | 4651 | 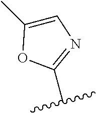 [SMILES: *c1ncc(C)o1] | H | 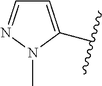 [SMILES: *c1ccnn1C] | H | F |
| 1652 | 4652 | 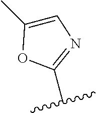 [SMILES: *c1ncc(C)o1] | CH₃ | 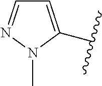 [SMILES: *c1ccnn1C] | H | F |
| 1653 | 4653 | 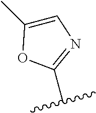 [SMILES: *c1ncc(C)o1] | H | 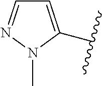 [SMILES: *c1ccnn1C] | F | H |
| 1654 | 4654 | 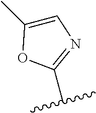 [SMILES: *c1ncc(C)o1] | CH₃ | 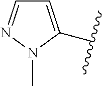 [SMILES: *c1ccnn1C] | F | H |
| 1655 | 4655 | 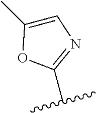 [SMILES: *c1ncc(C)o1] | H | 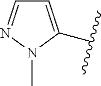 [SMILES: *c1ccnn1C] | H | Cl |
| 1656 | 4656 | 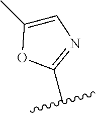 [SMILES: *c1ncc(C)o1] | CH₃ | 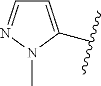 [SMILES: *c1ccnn1C] | H | Cl |
| 1657 | 4657 | 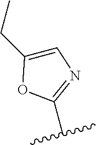 [SMILES: *c1ncc(CC)o1] | H | 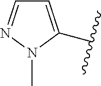 [SMILES: *c1ccnn1C] | H | H |
| 1658 | 4658 | 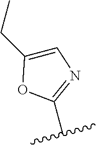 [SMILES: *c1ncc(CC)o1] | CH₃ | 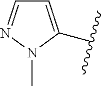 [SMILES: *c1ccnn1C] | H | H |
| 1659 | 4659 | 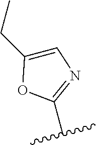 [SMILES: *c1ncc(CC)o1] | H | 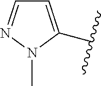 [SMILES: *c1ccnn1C] | H | F |
| 1660 | 4660 | 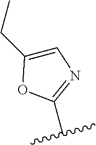 [SMILES: *c1ncc(CC)o1] | CH₃ | 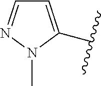 [SMILES: *c1ccnn1C] | H | F |
| 1661 | 4661 | 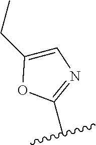 [SMILES: *c1ncc(CC)o1] | H | 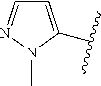 [SMILES: *c1ccnn1C] | F | H |
| 1662 | 4662 | 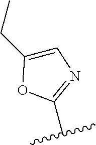 [SMILES: *c1ncc(CC)o1] | CH₃ | 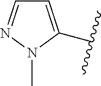 [SMILES: *c1ccnn1C] | F | H |
| 1663 | 4663 | 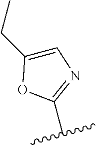 [SMILES: *c1ncc(CC)o1] | H | 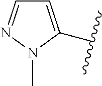 [SMILES: *c1ccnn1C] | H | Cl |
| 1664 | 4664 | 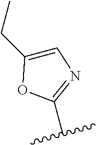 [SMILES: *c1ncc(CC)o1] | CH₃ | 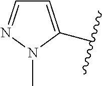 [SMILES: *c1ccnn1C] | H | Cl |
| 1665 | 4665 | 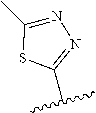 [SMILES: *c1nnc(C)s1] | H | 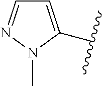 [SMILES: *c1ccnn1C] | H | H |
| 1666 | 4666 | 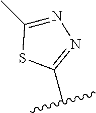 [SMILES: *c1nnc(C)s1] | CH₃ | 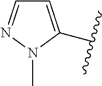 [SMILES: *c1ccnn1C] | H | H |
| 1667 | 4667 | 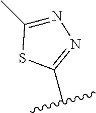 [SMILES: *c1nnc(C)s1] | H | 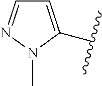 [SMILES: *c1ccnn1C] | H | F |
| 1668 | 4668 | 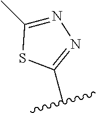 [SMILES: *c1nnc(C)s1] | CH₃ | 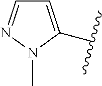 [SMILES: *c1ccnn1C] | H | F |
| 1669 | 4669 | 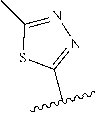 [SMILES: *c1nnc(C)s1] | H | 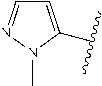 [SMILES: *c1ccnn1C] | F | H |
| 1670 | 4670 | 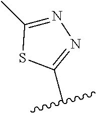 [SMILES: *c1nnc(C)s1] | CH₃ | 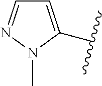 [SMILES: *c1ccnn1C] | F | H |
| 1671 | 4671 | 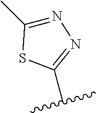 [SMILES: *c1nnc(C)s1] | H | 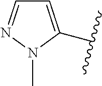 [SMILES: *c1ccnn1C] | H | Cl |
| 1672 | 4672 | 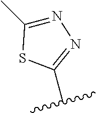 [SMILES: *c1nnc(C)s1] | CH₃ | 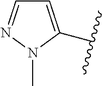 [SMILES: *c1ccnn1C] | H | Cl |
| 1673 | 4673 | 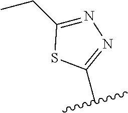 [SMILES: *c1nnc(CC)s1] | H | 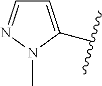 [SMILES: *c1ccnn1C] | H | H |
| 1674 | 4674 | 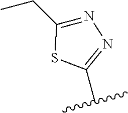 [SMILES: *c1nnc(CC)s1] | CH₃ | 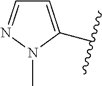 [SMILES: *c1ccnn1C] | H | H |
| 1675 | 4675 | 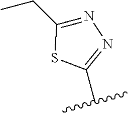 [SMILES: *c1nnc(CC)s1] | H | 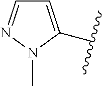 [SMILES: *c1ccnn1C] | H | F |
| 1676 | 4676 | 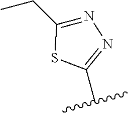 [SMILES: *c1nnc(CC)s1] | CH₃ | 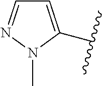 [SMILES: *c1ccnn1C] | H | F |
| 1677 | 4677 | 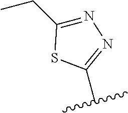 [SMILES: *c1nnc(CC)s1] | H | 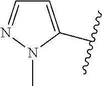 [SMILES: *c1ccnn1C] | F | H |
| 1678 | 4678 | 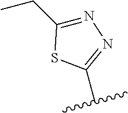 [SMILES: *c1nnc(CC)s1] | CH₃ | 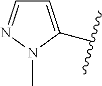 [SMILES: *c1ccnn1C] | F | H |
| 1679 | 4679 | 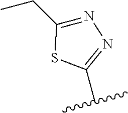 [SMILES: *c1nnc(CC)s1] | H | 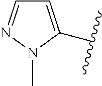 [SMILES: *c1ccnn1C] | H | Cl |
| 1680 | 4680 | 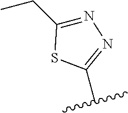 [SMILES: *c1nnc(CC)s1] | CH₃ | 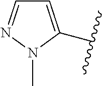 [SMILES: *c1ccnn1C] | H | Cl |
| 1681 | 4681 | 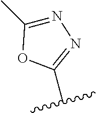 [SMILES: *c1nnc(C)o1] | H | 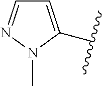 [SMILES: *c1ccnn1C] | H | H |
| 1682 | 4682 | 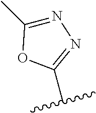 [SMILES: *c1nnc(C)o1] | CH₃ | 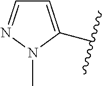 [SMILES: *c1ccnn1C] | H | H |
| 1683 | 4683 | 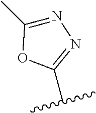 [SMILES: *c1nnc(C)o1] | H | 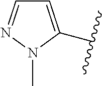 [SMILES: *c1ccnn1C] | H | F |
| 1684 | 4684 | 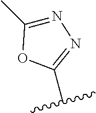 [SMILES: *c1nnc(C)o1] | CH₃ | 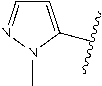 [SMILES: *c1ccnn1C] | H | F |
| 1685 | 4685 | 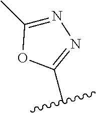 [SMILES: *c1nnc(C)o1] | H | 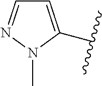 [SMILES: *c1ccnn1C] | F | H |
| 1686 | 4686 | 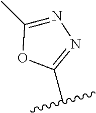 [SMILES: *c1nnc(C)o1] | CH₃ | 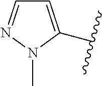 [SMILES: *c1ccnn1C] | F | H |
| 1687 | 4687 | 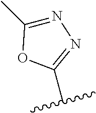 [SMILES: *c1nnc(C)o1] | H | 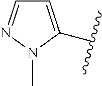 [SMILES: *c1ccnn1C] | H | Cl |
| 1689 | 4688 | 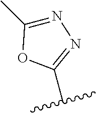 [SMILES: *c1nnc(C)o1] | CH₃ | 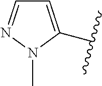 [SMILES: *c1ccnn1C] | H | Cl |
| 1690 | 4689 | 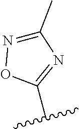 [SMILES: *c1nc(C)no1] | H | 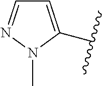 [SMILES: *c1ccnn1C] | H | H |
| 1691 | 4690 | 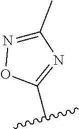 [SMILES: *c1nc(C)no1] | CH₃ | 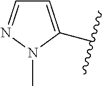 [SMILES: *c1ccnn1C] | H | H |
| 1692 | 4691 | 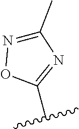 [SMILES: *c1nc(C)no1] | H | 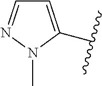 [SMILES: *c1ccnn1C] | H | F |
| 1693 | 4692 | 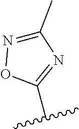 [SMILES: *c1nc(C)no1] | CH₃ | 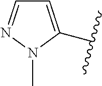 [SMILES: *c1ccnn1C] | H | F |
| 1694 | 4693 | 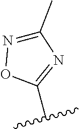 [SMILES: *c1nc(C)no1] | H | 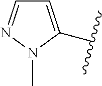 [SMILES: *c1ccnn1C] | F | H |
| 1695 | 4694 | 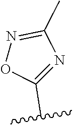 [SMILES: *c1nc(C)no1] | CH₃ | 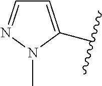 [SMILES: *c1ccnn1C] | F | H |
| 1696 | 4695 | 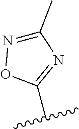 [SMILES: *c1nc(C)no1] | H | 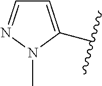 [SMILES: *c1ccnn1C] | H | Cl |
| 1697 | 4696 | 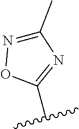 [SMILES: *c1nc(C)no1] | CH₃ | 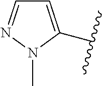 [SMILES: *c1ccnn1C] | H | Cl |
| 1698 | 4697 | 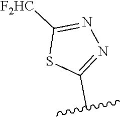 [SMILES: *c1nnc(C(F)F)s1] | H | 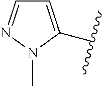 [SMILES: *c1ccnn1C] | H | H |
| 1699 | 4698 | 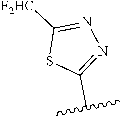 [SMILES: *c1nnc(C(F)F)s1] | CH₃ | 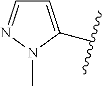 [SMILES: *c1ccnn1C] | H | H |
| 1700 | 4699 | 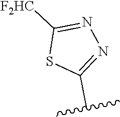 [SMILES: *c1nnc(C(F)F)s1] | H | 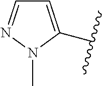 [SMILES: *c1ccnn1C] | H | F |
| 1701 | 4700 | 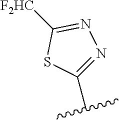 [SMILES: *c1nnc(C(F)F)s1] | CH₃ | 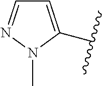 [SMILES: *c1ccnn1C] | H | F |
| 1702 | 4701 | 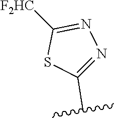 [SMILES: *c1nnc(C(F)F)s1] | H | 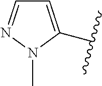 [SMILES: *c1ccnn1C] | F | H |
| 1703 | 4702 | 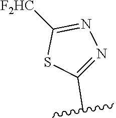 [SMILES: *c1nnc(C(F)F)s1] | CH₃ | 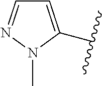 [SMILES: *c1ccnn1C] | F | H |
| 1704 | 4703 | 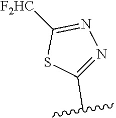 [SMILES: *c1nnc(C(F)F)s1] | H | 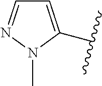 [SMILES: *c1ccnn1C] | H | Cl |
| 1705 | 4704 | 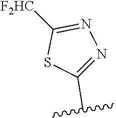 [SMILES: *c1nnc(C(F)F)s1] | CH₃ | 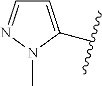 [SMILES: *c1ccnn1C] | H | Cl |
| 1706 | 4705 | 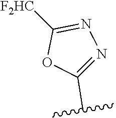 [SMILES: *c1nnc(C(F)F)o1] | H | 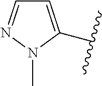 [SMILES: *c1ccnn1C] | H | H |
| 1707 | 4706 | 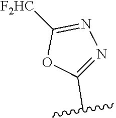 [SMILES: *c1nnc(C(F)F)o1] | CH₃ | 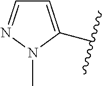 [SMILES: *c1ccnn1C] | H | H |
| 1708 | 4707 | 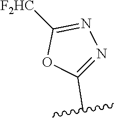 [SMILES: *c1nnc(C(F)F)o1] | H | 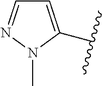 [SMILES: *c1ccnn1C] | H | F |
| 1709 | 4708 | 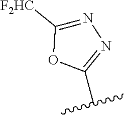 [SMILES: *c1nnc(C(F)F)o1] | CH₃ | 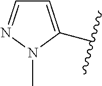 [SMILES: *c1ccnn1C] | H | F |
| 1710 | 4709 | 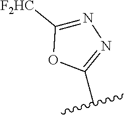 [SMILES: *c1nnc(C(F)F)o1] | H | 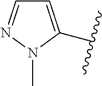 [SMILES: *c1ccnn1C] | F | H |
| 1711 | 4710 | 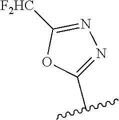 [SMILES: *c1nnc(C(F)F)o1] | CH₃ | 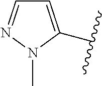 [SMILES: *c1ccnn1C] | F | H |
| 1712 | 4711 | 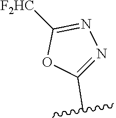 [SMILES: *c1nnc(C(F)F)o1] | H | 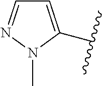 [SMILES: *c1ccnn1C] | H | Cl |
| 1713 | 4712 | 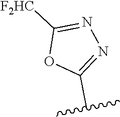 [SMILES: *c1nnc(C(F)F)o1] | CH₃ | 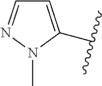 [SMILES: *c1ccnn1C] | H | Cl |
| 1714 | 4713 | 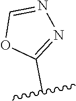 [SMILES: *c1nnco1] | H | 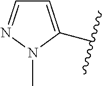 [SMILES: *c1ccnn1C] | H | H |
| 1715 | 4714 | 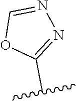 [SMILES: *c1nnco1] | CH₃ | 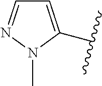 [SMILES: *c1ccnn1C] | H | H |
| 1716 | 4715 | 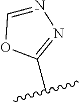 [SMILES: *c1nnco1] | H | 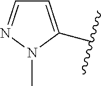 [SMILES: *c1ccnn1C] | H | F |
| 1717 | 4716 | 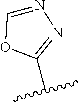 [SMILES: *c1nnco1] | CH₃ | 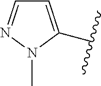 [SMILES: *c1ccnn1C] | H | F |
| 1718 | 4717 | 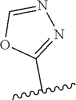 [SMILES: *c1nnco1] | H | 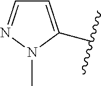 [SMILES: *c1ccnn1C] | F | H |
| 1719 | 4718 | 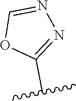 [SMILES: *c1nnco1] | CH₃ | 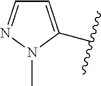 [SMILES: *c1ccnn1C] | F | H |
| 1720 | 4719 | 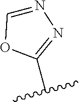 [SMILES: *c1nnco1] | H | 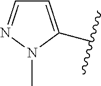 [SMILES: *c1ccnn1C] | H | Cl |
| 1721 | 4720 | 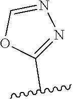 [SMILES: *c1nnco1] | CH₃ | 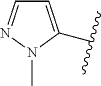 [SMILES: *c1ccnn1C] | H | Cl |
| 1722 | 4721 | 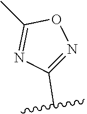 [SMILES: *c1noc(C)n1] | H | 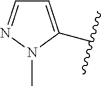 [SMILES: *c1ccnn1C] | H | H |
| 1723 | 4722 | 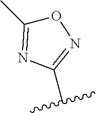 [SMILES: *c1noc(C)n1] | CH₃ | 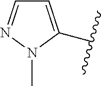 [SMILES: *c1ccnn1C] | H | H |
| 1724 | 4723 | 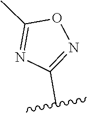 [SMILES: *c1noc(C)n1] | H | 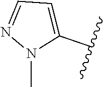 [SMILES: *c1ccnn1C] | H | F |
| 1725 | 4724 | 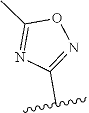 [SMILES: *c1noc(C)n1] | CH₃ | 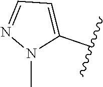 [SMILES: *c1ccnn1C] | H | F |
| 1726 | 4725 | 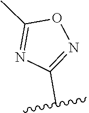 [SMILES: *c1noc(C)n1] | H | 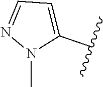 [SMILES: *c1ccnn1C] | F | H |
| 1727 | 4726 | 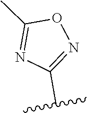 [SMILES: *c1noc(C)n1] | CH₃ | 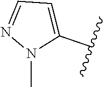 [SMILES: *c1ccnn1C] | F | H |
| 1728 | 4727 | 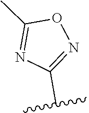 [SMILES: *c1noc(C)n1] | H | 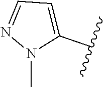 [SMILES: *c1ccnn1C] | H | Cl |
| 1729 | 4728 | 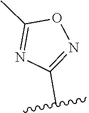 [SMILES: *c1noc(C)n1] | CH₃ | 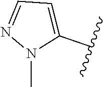 [SMILES: *c1ccnn1C] | H | Cl |

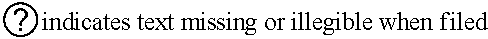

In some embodiments, the compound of formula (A), formula (I), or formula (II) is a compound having any one of formula 2001-2337, or a pharmaceutically acceptable salt, solvate, hydrate, cocrystal, or prodrug thereof:

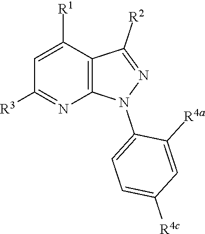

| Cpd. # | R¹ | R² | R³ | R^{4a} | R^{4c} |
|---|---|---|---|---|---|
| 2001 | 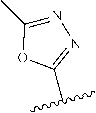 [SMILES: *c1nnc(C)o1] | CHF₂ | 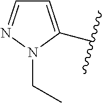 [SMILES: *c1ccnn1CC] | H | H |
| 2002 | 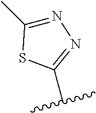 [SMILES: *c1nnc(C)s1] | CHF₂ | 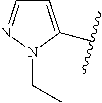 [SMILES: *c1ccnn1CC] | H | H |
| 2003 | 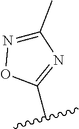 [SMILES: *c1nc(C)no1] | CHF₂ | 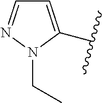 [SMILES: *c1ccnn1CC] | H | H |
| 2004 | 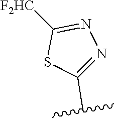 [SMILES: *c1nnc(C(F)F)s1] | CHF₂ | 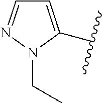 [SMILES: *c1ccnn1CC] | H | H |
| 2005 | 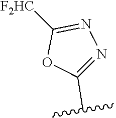 [SMILES: *c1nnc(C(F)F)o1] | CHF₂ | 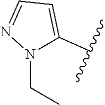 [SMILES: *c1ccnn1CC] | H | H |
| 2006 | 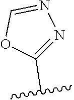 [SMILES: *c1nnco1] | CHF₂ | 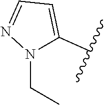 [SMILES: *c1ccnn1CC] | H | H |
| 2007 | 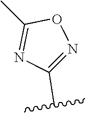 [SMILES: *c1noc(C)n1] | CHF₂ | 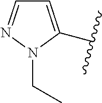 [SMILES: *c1ccnn1CC] | H | H |
| 2008 | 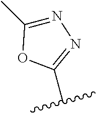 [SMILES: *c1nnc(C)o1] | CF₃ | 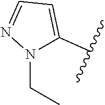 [SMILES: *c1ccnn1CC] | H | H |
| 2009 | 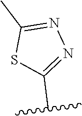 [SMILES: *c1nnc(C)s1] | CF₃ | 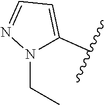 [SMILES: *c1ccnn1CC] | H | H |
| 2010 | 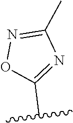 [SMILES: *c1nc(C)no1] | CF₃ | 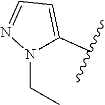 [SMILES: *c1ccnn1CC] | H | H |
| 2011 | 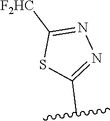 [SMILES: *c1nnc(C(F)F)s1] | CF₃ | 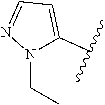 [SMILES: *c1ccnn1CC] | H | H |
| 2012 | 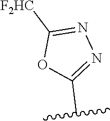 [SMILES: *c1nnc(C(F)F)o1] | CF₃ | 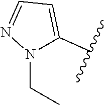 [SMILES: *c1ccnn1CC] | H | H |
| 2013 | 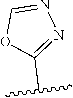 [SMILES: *c1nnco1] | CF₃ | 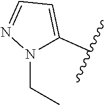 [SMILES: *c1ccnn1CC] | H | H |
| 2014 | 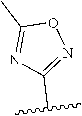 [SMILES: *c1noc(C)n1] | CF₃ | 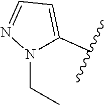 [SMILES: *c1ccnn1CC] | H | H |
| 2015 | 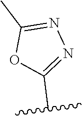 [SMILES: *c1nnc(C)o1] | CHF₂ | 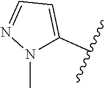 [SMILES: *c1ccnn1C] | H | H |
| 2016 | 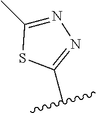 [SMILES: *c1nnc(C)s1] | CHF₂ | 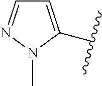 [SMILES: *c1ccnn1C] | H | H |
| 2017 | 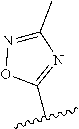 [SMILES: *c1nc(C)no1] | CHF₂ | 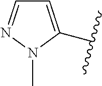 [SMILES: *c1ccnn1C] | H | H |
| 2018 | 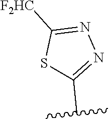 [SMILES: *c1nnc(C(F)F)s1] | CHF₂ | 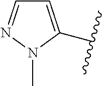 [SMILES: *c1ccnn1C] | H | H |
| 2019 | 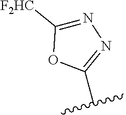 [SMILES: *c1nnc(C(F)F)o1] | CHF₂ | 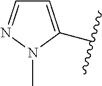 [SMILES: *c1ccnn1C] | H | H |
| 2020 | 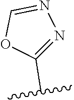 [SMILES: *c1nnco1] | CHF₂ | 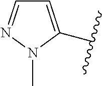 [SMILES: *c1ccnn1C] | H | H |
| 2021 | 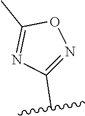 [SMILES: *c1noc(C)n1] | CHF₂ | 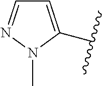 [SMILES: *c1ccnn1C] | H | H |
| 2022 | 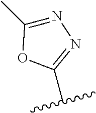 [SMILES: *c1nnc(C)o1] | CF₃ | 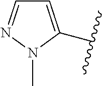 [SMILES: *c1ccnn1C] | H | H |
| 2023 | 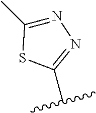 [SMILES: *c1nnc(C)s1] | CF₃ | 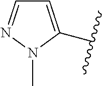 [SMILES: *c1ccnn1C] | H | H |
| 2024 | 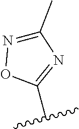 [SMILES: *c1nc(C)no1] | CF₃ | 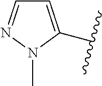 [SMILES: *c1ccnn1C] | H | H |
| 2025 | 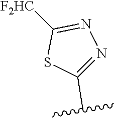 [SMILES: *c1nnc(C(F)F)s1] | CF₃ | 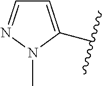 [SMILES: *c1ccnn1C] | H | H |
| 2026 | 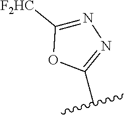 [SMILES: *c1nnc(C(F)F)o1] | CF₃ | 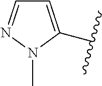 [SMILES: *c1ccnn1C] | H | H |
| 2027 | 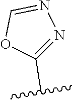 [SMILES: *c1nnco1] | CF₃ | 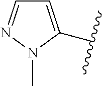 [SMILES: *c1ccnn1C] | H | H |
| 2028 | 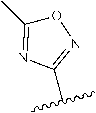 [SMILES: *c1noc(C)n1] | CF₃ | 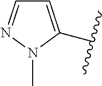 [SMILES: *c1ccnn1C] | H | H |
| 2029 | 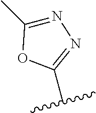 [SMILES: *c1nnc(C)o1] | CHF₂ | 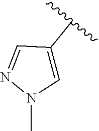 [SMILES: *c1cnn(C)c1] | H | H |
| 2030 | 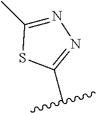 [SMILES: *c1nnc(C)s1] | CHF₂ | 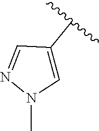 [SMILES: *c1cnn(C)c1] | H | H |
| 2031 | 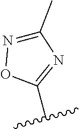 [SMILES: *c1nc(C)no1] | CHF₂ | 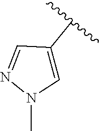 [SMILES: *c1cnn(C)c1] | H | H |
| 2032 | 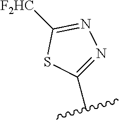 [SMILES: *c1nnc(C(F)F)s1] | CHF₂ | 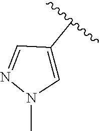 [SMILES: *c1cnn(C)c1] | H | H |
| 2033 | 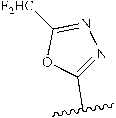 [SMILES: *c1nnc(C(F)F)o1] | CHF₂ | 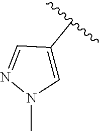 [SMILES: *c1cnn(C)c1] | H | H |
| 2034 | 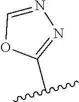 [SMILES: *c1nnco1] | CHF₂ | 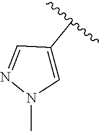 [SMILES: *c1cnn(C)c1] | H | H |
| 2035 | 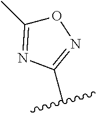 [SMILES: *c1noc(C)n1] | CHF₂ | 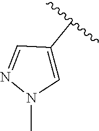 [SMILES: *c1cnn(C)c1] | H | H |
| 2036 | 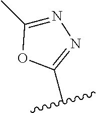 [SMILES: *c1nnc(C)o1] | CF₃ | 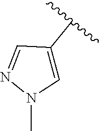 [SMILES: *c1cnn(C)c1] | H | H |
| 2037 | 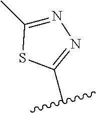 [SMILES: *c1nnc(C)s1] | CF₃ | 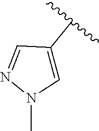 [SMILES: *c1cnn(C)c1] | H | H |
| 2038 | 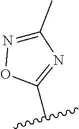 [SMILES: *c1nc(C)no1] | CF₃ | 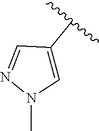 [SMILES: *c1cnn(C)c1] | H | H |
| 2039 | 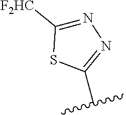 [SMILES: *c1nnc(C(F)F)s1] | CF₃ | 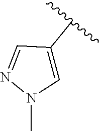 [SMILES: *c1cnn(C)c1] | H | H |
| 2040 | 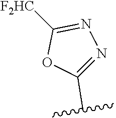 [SMILES: *c1nnc(C(F)F)o1] | CF₃ | 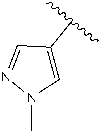 [SMILES: *c1cnn(C)c1] | H | H |
| 2041 | 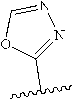 [SMILES: *c1nnco1] | CF₃ | 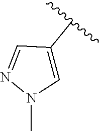 [SMILES: *c1cnn(C)c1] | H | H |
| 2042 | 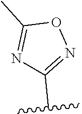 [SMILES: *c1noc(C)n1] | CF₃ | 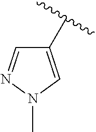 [SMILES: *c1cnn(C)c1] | H | H |
| 2043 | 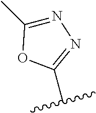 [SMILES: *c1nnc(C)o1] | CHF₂ | 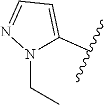 [SMILES: *c1ccnn1CC] | H | F |
| 2044 | 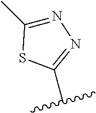 [SMILES: *c1nnc(C)s1] | CHF₂ | 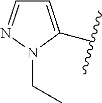 [SMILES: *c1ccnn1CC] | H | F |
| 2045 | 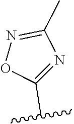 [SMILES: *c1nc(C)no1] | CHF₂ | 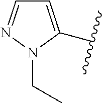 [SMILES: *c1ccnn1CC] | H | F |
| 2046 | 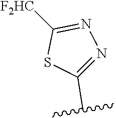 [SMILES: *c1nnc(C(F)F)s1] | CHF₂ | 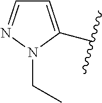 [SMILES: *c1ccnn1CC] | H | F |
| 2046 | 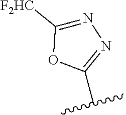 [SMILES: *c1nnc(C(F)F)o1] | CHF₂ | 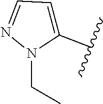 [SMILES: *c1ccnn1CC] | H | F |
| 2047 | 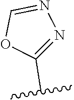 [SMILES: *c1nnco1] | CHF₂ | 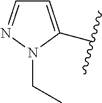 [SMILES: *c1ccnn1CC] | H | F |
| 2048 | 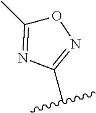 [SMILES: *c1noc(C)n1] | CHF₂ | 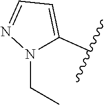 [SMILES: *c1ccnn1CC] | H | F |
| 2049 | 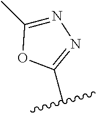 [SMILES: *c1nnc(C)o1] | CF₃ | 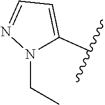 [SMILES: *c1ccnn1CC] | H | F |
| 2050 | 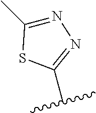 [SMILES: *c1nnc(C)s1] | CF₃ | 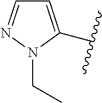 [SMILES: *c1ccnn1CC] | H | F |
| 2051 | 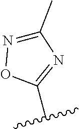 [SMILES: *c1nc(C)no1] | CF₃ | 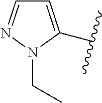 [SMILES: *c1ccnn1CC] | H | F |
| 2052 | 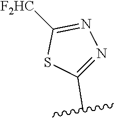 [SMILES: *c1nnc(C(F)F)s1] | CF₃ | 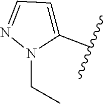 [SMILES: *c1ccnn1CC] | H | F |
| 2053 | 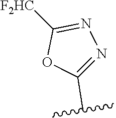 [SMILES: *c1nnc(C(F)F)o1] | CF₃ | 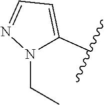 [SMILES: *c1ccnn1CC] | H | F |
| 2054 | 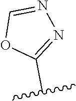 [SMILES: *c1nnco1] | CF₃ | 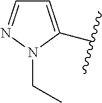 [SMILES: *c1ccnn1CC] | H | F |
| 2055 | 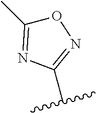 [SMILES: *c1noc(C)n1] | CF₃ | 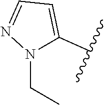 [SMILES: *c1ccnn1CC] | H | F |
| 2056 | 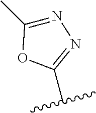 [SMILES: *c1nnc(C)o1] | CHF₂ | 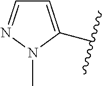 [SMILES: *c1ccnn1C] | H | F |
| 2057 | 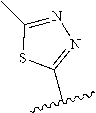 [SMILES: *c1nnc(C)s1] | CHF₂ | 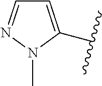 [SMILES: *c1ccnn1C] | H | F |
| 2058 | 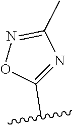 [SMILES: *c1nc(C)no1] | CHF₂ | 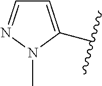 [SMILES: *c1ccnn1C] | H | F |
| 2059 | 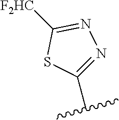 [SMILES: *c1nnc(C(F)F)s1] | CHF₂ | 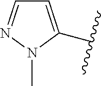 [SMILES: *c1ccnn1C] | H | F |
| 2060 | 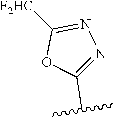 [SMILES: *c1nnc(C(F)F)o1] | CHF₂ | 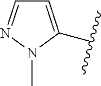 [SMILES: *c1ccnn1C] | H | F |
| 2061 | 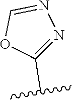 [SMILES: *c1nnco1] | CHF₂ | 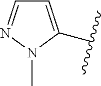 [SMILES: *c1ccnn1C] | H | F |
| 2062 | 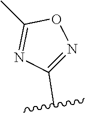 [SMILES: *c1noc(C)n1] | CHF₂ | 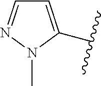 [SMILES: *c1ccnn1C] | H | F |
| 2063 | 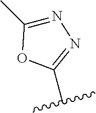 [SMILES: *c1nnc(C)o1] | CF₃ | 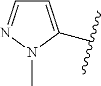 [SMILES: *c1ccnn1C] | H | F |
| 2064 | 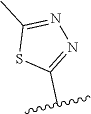 [SMILES: *c1nnc(C)s1] | CF₃ | 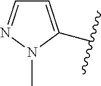 [SMILES: *c1ccnn1C] | H | F |
| 2065 | 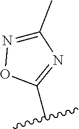 [SMILES: *c1nc(C)no1] | CF₃ | 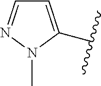 [SMILES: *c1ccnn1C] | H | F |
| 2066 | 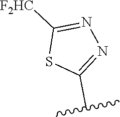 [SMILES: *c1nnc(C(F)F)s1] | CF₃ | 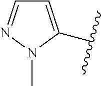 [SMILES: *c1ccnn1C] | H | F |
| 2067 | 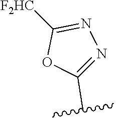 [SMILES: *c1nnc(C(F)F)o1] | CF₃ | 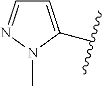 [SMILES: *c1ccnn1C] | H | F |
| 2068 | 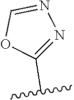 [SMILES: *c1nnco1] | CF₃ | 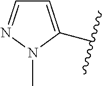 [SMILES: *c1ccnn1C] | H | F |
| 2069 | 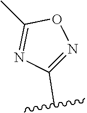 [SMILES: *c1noc(C)n1] | CF₃ | 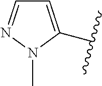 [SMILES: *c1ccnn1C] | H | F |
| 2070 | 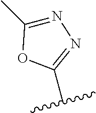 [SMILES: *c1nnc(C)o1] | CHF₂ | 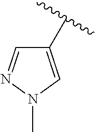 [SMILES: *c1cnn(C)c1] | H | F |
| 2071 | 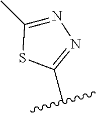 [SMILES: *c1nnc(C)s1] | CHF₂ | 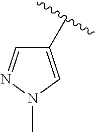 [SMILES: *c1cnn(C)c1] | H | F |
| 2072 | 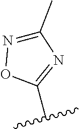 [SMILES: *c1nc(C)no1] | CHF₂ | 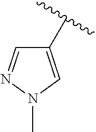 [SMILES: *c1cnn(C)c1] | H | F |
| 2073 | 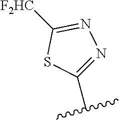 [SMILES: *c1nnc(C(F)F)s1] | CHF₂ | 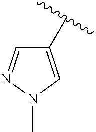 [SMILES: *c1cnn(C)c1] | H | F |
| 2074 | 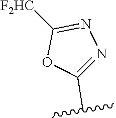 [SMILES: *c1nnc(C(F)F)o1] | CHF₂ | 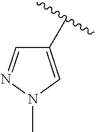 [SMILES: *c1cnn(C)c1] | H | F |
| 2075 | 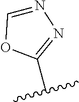 [SMILES: *c1nnco1] | CHF₂ | 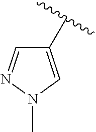 [SMILES: *c1cnn(C)c1] | H | F |
| 2076 | 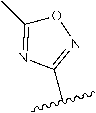 [SMILES: *c1noc(C)n1] | CHF₂ | 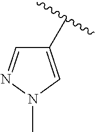 [SMILES: *c1cnn(C)c1] | H | F |
| 2077 | 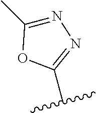 [SMILES: *c1nnc(C)o1] | CF₃ | 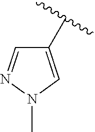 [SMILES: *c1cnn(C)c1] | H | F |
| 2078 | 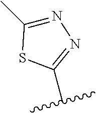 [SMILES: *c1nnc(C)s1] | CF₃ | 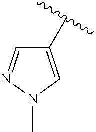 [SMILES: *c1cnn(C)c1] | H | F |
| 2079 | 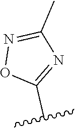 [SMILES: *c1nc(C)no1] | CF₃ | 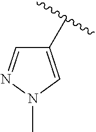 [SMILES: *c1cnn(C)c1] | H | F |
| 2080 | 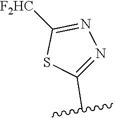 [SMILES: *c1nnc(C(F)F)s1] | CF₃ | 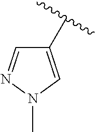 [SMILES: *c1cnn(C)c1] | H | F |
| 2081 | 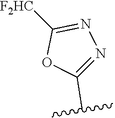 [SMILES: *c1nnc(C(F)F)o1] | CF₃ | 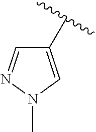 [SMILES: *c1cnn(C)c1] | H | F |
| 2082 | 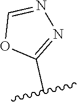 [SMILES: *c1nnco1] | CF₃ | 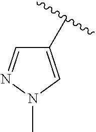 [SMILES: *c1cnn(C)c1] | H | F |
| 2083 | 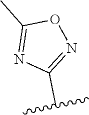 [SMILES: *c1noc(C)n1] | CF₃ | 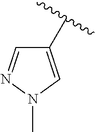 [SMILES: *c1cnn(C)c1] | H | F |
| 2084 | 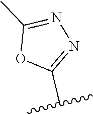 [SMILES: *c1nnc(C)o1] | CHF₂ | 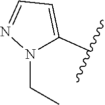 [SMILES: *c1ccnn1CC] | F | H |
| 2085 | 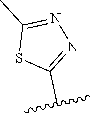 [SMILES: *c1nnc(C)s1] | CHF₂ | 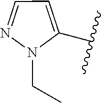 [SMILES: *c1ccnn1CC] | F | H |
| 2086 | 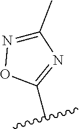 [SMILES: *c1nc(C)no1] | CHF₂ | 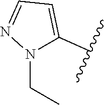 [SMILES: *c1ccnn1CC] | F | H |
| 2087 | 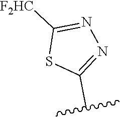 [SMILES: *c1nnc(C(F)F)s1] | CHF₂ | 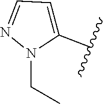 [SMILES: *c1ccnn1CC] | F | H |
| 2088 | 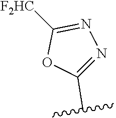 [SMILES: *c1nnc(C(F)F)o1] | CHF₂ | 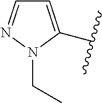 [SMILES: *c1ccnn1CC] | F | H |
| 2089 | 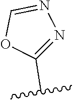 [SMILES: *c1nnco1] | CHF₂ | 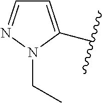 [SMILES: *c1ccnn1CC] | F | H |
| 2090 | 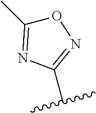 [SMILES: *c1noc(C)n1] | CHF₂ | 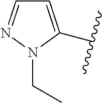 [SMILES: *c1ccnn1CC] | F | H |
| 2091 | 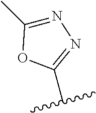 [SMILES: *c1nnc(C)o1] | CF₃ | 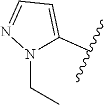 [SMILES: *c1ccnn1CC] | F | H |
| 2092 | 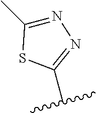 [SMILES: *c1nnc(C)s1] | CF₃ | 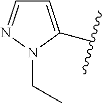 [SMILES: *c1ccnn1CC] | F | H |
| 2093 | 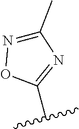 [SMILES: *c1nc(C)no1] | CF₃ | 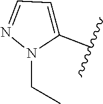 [SMILES: *c1ccnn1CC] | F | H |
| 2094 | 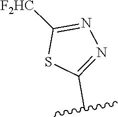 [SMILES: *c1nnc(C(F)F)s1] | CF₃ | 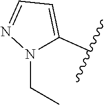 [SMILES: *c1ccnn1CC] | F | H |
| 2095 | 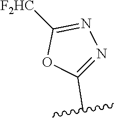 [SMILES: *c1nnc(C(F)F)o1] | CF₃ | 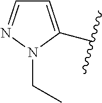 [SMILES: *c1ccnn1CC] | F | H |
| 2096 | 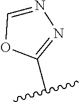 [SMILES: *c1nnco1] | CF₃ | 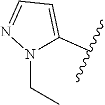 [SMILES: *c1ccnn1CC] | F | H |
| 2097 | 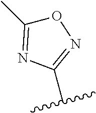 [SMILES: *c1noc(C)n1] | CF₃ | 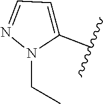 [SMILES: *c1ccnn1CC] | F | H |
| 2098 | 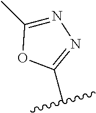 [SMILES: *c1nnc(C)o1] | CHF₂ | 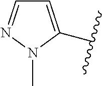 [SMILES: *c1ccnn1C] | F | H |
| 2099 | 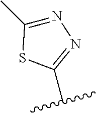 [SMILES: *c1nnc(C)s1] | CHF₂ | 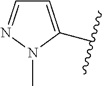 [SMILES: *c1ccnn1C] | F | H |
| 2100 | 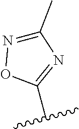 [SMILES: *c1nc(C)no1] | CHF₂ | 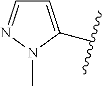 [SMILES: *c1ccnn1C] | F | H |
| 2101 | 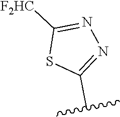 [SMILES: *c1nnc(C(F)F)s1] | CHF₂ | 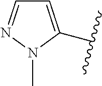 [SMILES: *c1ccnn1C] | F | H |
| 2102 | 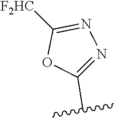 [SMILES: *c1nnc(C(F)F)o1] | CHF₂ | 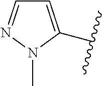 [SMILES: *c1ccnn1C] | F | H |
| 2103 | 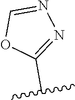 [SMILES: *c1nnco1] | CHF₂ | 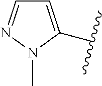 [SMILES: *c1ccnn1C] | F | H |
| 2104 | 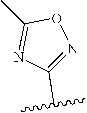 [SMILES: *c1noc(C)n1] | CHF₂ | 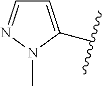 [SMILES: *c1ccnn1C] | F | H |
| 2105 | 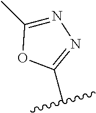 [SMILES: *c1nnc(C)o1] | CF₃ | 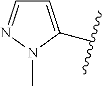 [SMILES: *c1ccnn1C] | F | H |
| 2106 | 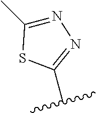 [SMILES: *c1nnc(C)s1] | CF₃ | 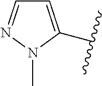 [SMILES: *c1ccnn1C] | F | H |
| 2107 | 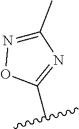 [SMILES: *c1nc(C)no1] | CF₃ | 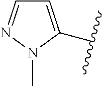 [SMILES: *c1ccnn1C] | F | H |
| 2108 | 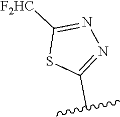 [SMILES: *c1nnc(C(F)F)s1] | CF₃ | 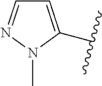 [SMILES: *c1ccnn1C] | F | H |
| 2109 | 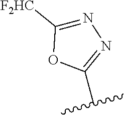 [SMILES: *c1nnc(C(F)F)o1] | CF₃ | 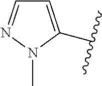 [SMILES: *c1ccnn1C] | F | H |
| 2110 | 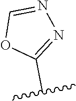 [SMILES: *c1nnco1] | CF₃ | 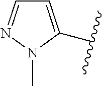 [SMILES: *c1ccnn1C] | F | H |
| 2111 | 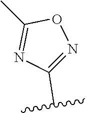 [SMILES: *c1noc(C)n1] | CF₃ | 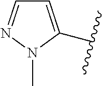 [SMILES: *c1ccnn1C] | F | H |
| 2112 | 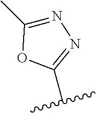 [SMILES: *c1nnc(C)o1] | CHF₂ | 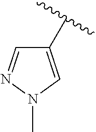 [SMILES: *c1cnn(C)c1] | F | H |
| 2113 | 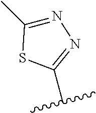 [SMILES: *c1nnc(C)s1] | CHF₂ | 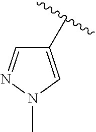 [SMILES: *c1cnn(C)c1] | F | H |
| 2114 | 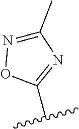 [SMILES: *c1nc(C)no1] | CHF₂ | 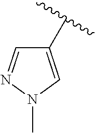 [SMILES: *c1cnn(C)c1] | F | H |
| 2115 | 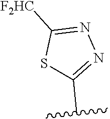 [SMILES: *c1nnc(C(F)F)s1] | CHF₂ | 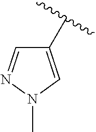 [SMILES: *c1cnn(C)c1] | F | H |
| 2116 | 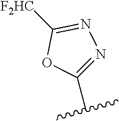 [SMILES: *c1nnc(C(F)F)o1] | CHF₂ | 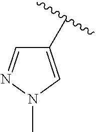 [SMILES: *c1cnn(C)c1] | F | H |
| 2117 | 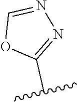 [SMILES: *c1nnco1] | CHF₂ | 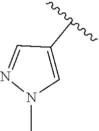 [SMILES: *c1cnn(C)c1] | F | H |
| 2118 | 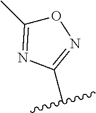 [SMILES: *c1noc(C)n1] | CHF₂ | 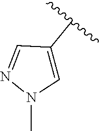 [SMILES: *c1cnn(C)c1] | F | H |
| 2119 | 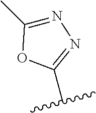 [SMILES: *c1nnc(C)o1] | CF₃ | 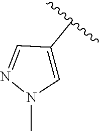 [SMILES: *c1cnn(C)c1] | F | H |
| 2120 | 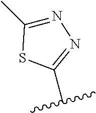 [SMILES: *c1nnc(C)s1] | CF₃ | 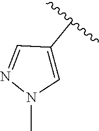 [SMILES: *c1cnn(C)c1] | F | H |
| 2121 | 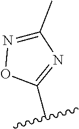 [SMILES: *c1nc(C)no1] | CF₃ | 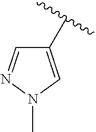 [SMILES: *c1cnn(C)c1] | F | H |
| 2122 | 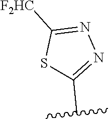 [SMILES: *c1nnc(C(F)F)s1] | CF₃ | 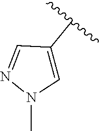 [SMILES: *c1cnn(C)c1] | F | H |
| 2123 | 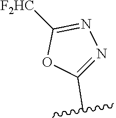 [SMILES: *c1nnc(C(F)F)o1] | CF₃ | 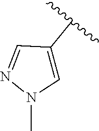 [SMILES: *c1cnn(C)c1] | F | H |
| 2124 | 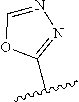 [SMILES: *c1nnco1] | CF₃ | 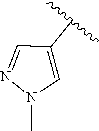 [SMILES: *c1cnn(C)c1] | F | H |
| 2125 | 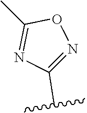 [SMILES: *c1noc(C)n1] | CF₃ | 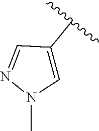 [SMILES: *c1cnn(C)c1] | F | H |
| 2126 | 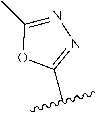 [SMILES: *c1nnc(C)o1] | CHF₂ | 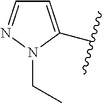 [SMILES: *c1ccnn1CC] | F | F |
| 2127 | 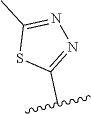 [SMILES: *c1nnc(C)s1] | CHF₂ | 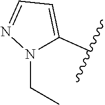 [SMILES: *c1ccnn1CC] | F | F |
| 2128 | 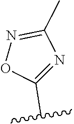 [SMILES: *c1nc(C)no1] | CHF₂ | 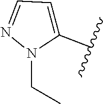 [SMILES: *c1ccnn1CC] | F | F |
| 2129 | 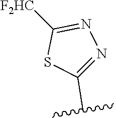 [SMILES: *c1nnc(C(F)F)s1] | CHF₂ | 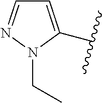 [SMILES: *c1ccnn1CC] | F | F |
| 2130 | 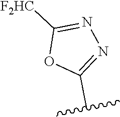 [SMILES: *c1nnc(C(F)F)o1] | CHF₂ | 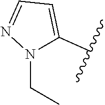 [SMILES: *c1ccnn1CC] | F | F |
| 2131 | 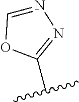 [SMILES: *c1nnco1] | CHF₂ | 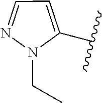 [SMILES: *c1ccnn1CC] | F | F |
| 2132 | 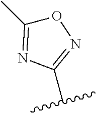 [SMILES: *c1noc(C)n1] | CHF₂ | 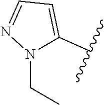 [SMILES: *c1ccnn1CC] | F | F |
| 2133 | 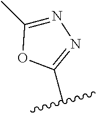 [SMILES: *c1nnc(C)o1] | CF₃ | 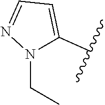 [SMILES: *c1ccnn1CC] | F | F |
| 2134 | 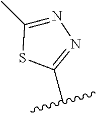 [SMILES: *c1nnc(C)s1] | CF₃ | 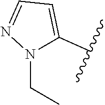 [SMILES: *c1ccnn1CC] | F | F |
| 2135 | 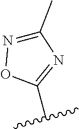 [SMILES: *c1nc(C)no1] | CF₃ | 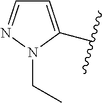 [SMILES: *c1ccnn1CC] | F | F |
| 2136 | 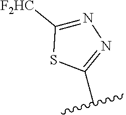 [SMILES: *c1nnc(C(F)F)s1] | CF₃ | 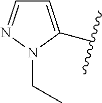 [SMILES: *c1ccnn1CC] | F | F |
| 2137 | 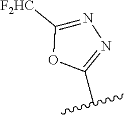 [SMILES: *c1nnc(C(F)F)o1] | CF₃ | 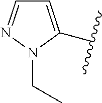 [SMILES: *c1ccnn1CC] | F | F |
| 2138 | 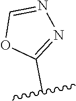 [SMILES: *c1nnco1] | CF₃ | 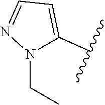 [SMILES: *c1ccnn1CC] | F | F |
| 2139 | 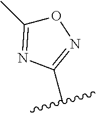 [SMILES: *c1noc(C)n1] | CF₃ | 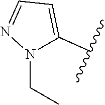 [SMILES: *c1ccnn1CC] | F | F |
| 2140 | 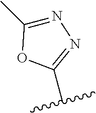 [SMILES: *c1nnc(C)o1] | CHF₂ | 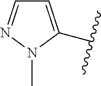 [SMILES: *c1ccnn1C] | F | F |
| 2141 | 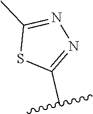 [SMILES: *c1nnc(C)s1] | CHF₂ | 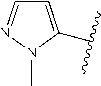 [SMILES: *c1ccnn1C] | F | F |
| 2142 | 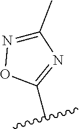 [SMILES: *c1nc(C)no1] | CHF₂ | 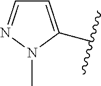 [SMILES: *c1ccnn1C] | F | F |
| 2143 | 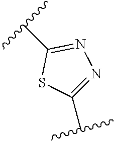 [SMILES: *c1nnc(*)s1] | CHF₂ | 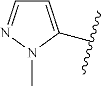 [SMILES: *c1ccnn1C] | F | F |
| 2144 | 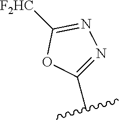 [SMILES: *c1nnc(C(F)F)o1] | CHF₂ | 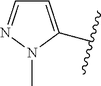 [SMILES: *c1ccnn1C] | F | F |
| 2145 | 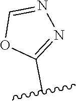 [SMILES: *c1nnco1] | CHF₂ | 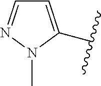 [SMILES: *c1ccnn1C] | F | F |
| 2146 | 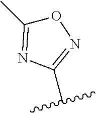 [SMILES: *c1noc(C)n1] | CHF₂ | 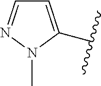 [SMILES: *c1ccnn1C] | F | F |
| 2147 | 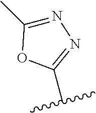 [SMILES: *c1nnc(C)o1] | CF₃ | 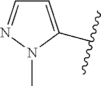 [SMILES: *c1ccnn1C] | F | F |
| 2148 | 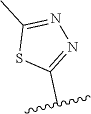 [SMILES: *c1nnc(C)s1] | CF₃ | 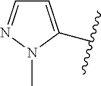 [SMILES: *c1ccnn1C] | F | F |
| 2149 | 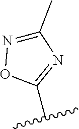 [SMILES: *c1nc(C)no1] | CF₃ | 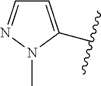 [SMILES: *c1ccnn1C] | F | F |
| 2150 | 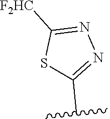 [SMILES: *c1nnc(C(F)F)s1] | CF₃ | 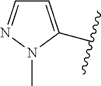 [SMILES: *c1ccnn1C] | F | F |
| 2151 | 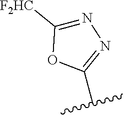 [SMILES: *c1nnc(C(F)F)o1] | CF₃ | 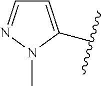 [SMILES: *c1ccnn1C] | F | F |
| 2152 | 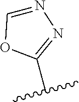 [SMILES: *c1nnco1] | CF₃ | 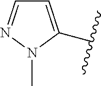 [SMILES: *c1ccnn1C] | F | F |
| 2153 | 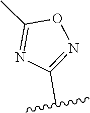 [SMILES: *c1noc(C)n1] | CF₃ | 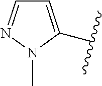 [SMILES: *c1ccnn1C] | F | F |
| 2154 | 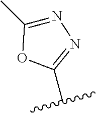 [SMILES: *c1nnc(C)o1] | CHF₂ | 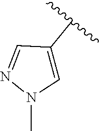 [SMILES: *c1cnn(C)c1] | F | F |
| 2155 | 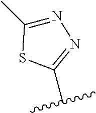 [SMILES: *c1nnc(C)s1] | CHF₂ | 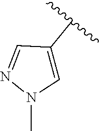 [SMILES: *c1cnn(C)c1] | F | F |
| 2156 | 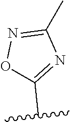 [SMILES: *c1nc(C)no1] | CHF₂ | 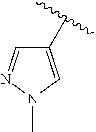 [SMILES: *c1cnn(C)c1] | F | F |
| 2157 | 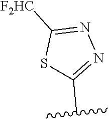 [SMILES: *c1nnc(C(F)F)s1] | CHF₂ | 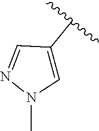 [SMILES: *c1cnn(C)c1] | F | F |
| 2158 | 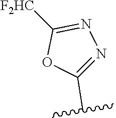 [SMILES: *c1nnc(C(F)F)o1] | CHF₂ | 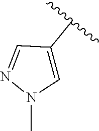 [SMILES: *c1cnn(C)c1] | F | F |
| 2159 | 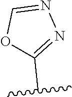 [SMILES: *c1nnco1] | CHF₂ | 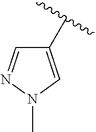 [SMILES: *c1cnn(C)c1] | F | F |
| 2160 | 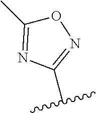 [SMILES: *c1noc(C)n1] | CHF₂ | 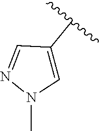 [SMILES: *c1cnn(C)c1] | F | F |
| 2161 | 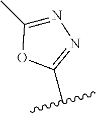 [SMILES: *c1nnc(C)o1] | CF₃ | 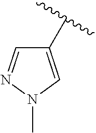 [SMILES: *c1cnn(C)c1] | F | F |
| 2162 | 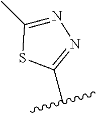 [SMILES: *c1nnc(C)s1] | CF₃ | 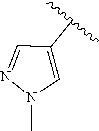 [SMILES: *c1cnn(C)c1] | F | F |
| 2163 | 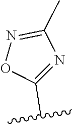 [SMILES: *c1nc(C)no1] | CF₃ | 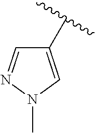 [SMILES: *c1cnn(C)c1] | F | F |
| 2164 | 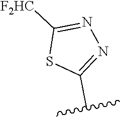 [SMILES: *c1nnc(C(F)F)s1] | CF₃ | 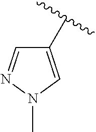 [SMILES: *c1cnn(C)c1] | F | F |
| 2165 | 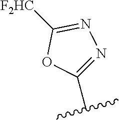 [SMILES: *c1nnc(C(F)F)o1] | CF₃ | 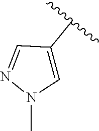 [SMILES: *c1cnn(C)c1] | F | F |
| 2166 | 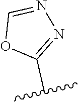 [SMILES: *c1nnco1] | CF₃ | 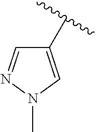 [SMILES: *c1cnn(C)c1] | F | F |
| 2167 | 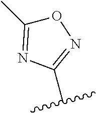 [SMILES: *c1noc(C)n1] | CF₃ | 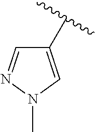 [SMILES: *c1cnn(C)c1] | F | F |
| 2168 | 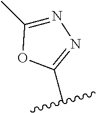 [SMILES: *c1nnc(C)o1] | CHF₂ | 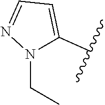 [SMILES: *c1ccnn1CC] | Cl | H |
| 2169 | 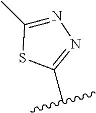 [SMILES: *c1nnc(C)s1] | CHF₂ | 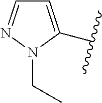 [SMILES: *c1ccnn1CC] | Cl | H |
| 2170 | 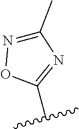 [SMILES: *c1nc(C)no1] | CHF₂ | 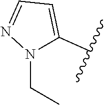 [SMILES: *c1ccnn1CC] | Cl | H |
| 2171 | 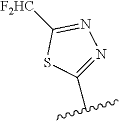 [SMILES: *c1nnc(C(F)F)s1] | CHF₂ | 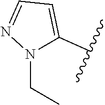 [SMILES: *c1ccnn1CC] | Cl | H |
| 2172 | 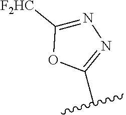 [SMILES: *c1nnc(C(F)F)o1] | CHF₂ | 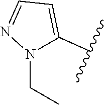 [SMILES: *c1ccnn1CC] | Cl | H |
| 2173 | 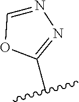 [SMILES: *c1nnco1] | CHF₂ | 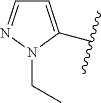 [SMILES: *c1ccnn1CC] | Cl | H |
| 2174 | 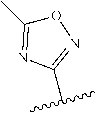 [SMILES: *c1noc(C)n1] | CHF₂ | 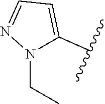 [SMILES: *c1ccnn1CC] | Cl | H |
| 2175 | 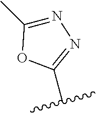 [SMILES: *c1nnc(C)o1] | CF₃ | 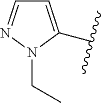 [SMILES: *c1ccnn1CC] | Cl | H |
| 2176 | 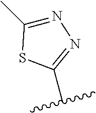 [SMILES: *c1nnc(C)s1] | CF₃ | 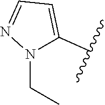 [SMILES: *c1ccnn1CC] | Cl | H |
| 2177 | 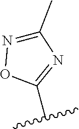 [SMILES: *c1nc(C)no1] | CF₃ | 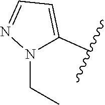 [SMILES: *c1ccnn1CC] | Cl | H |
| 2178 | 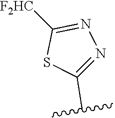 [SMILES: *c1nnc(C(F)F)s1] | CF₃ | 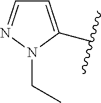 [SMILES: *c1ccnn1CC] | Cl | H |
| 2179 | 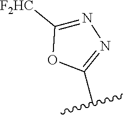 [SMILES: *c1nnc(C(F)F)o1] | CF₃ | 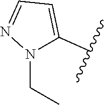 [SMILES: *c1ccnn1CC] | Cl | H |
| 2180 | 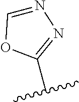 [SMILES: *c1nnco1] | CF₃ | 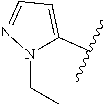 [SMILES: *c1ccnn1CC] | Cl | H |
| 2181 | 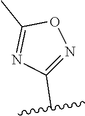 [SMILES: *c1noc(C)n1] | CF₃ | 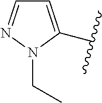 [SMILES: *c1ccnn1CC] | Cl | H |
| 2182 | 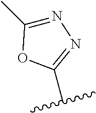 [SMILES: *c1nnc(C)o1] | CHF₂ | 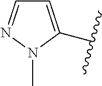 [SMILES: *c1ccnn1C] | Cl | H |
| 2183 | 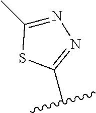 [SMILES: *c1nnc(C)s1] | CHF₂ | 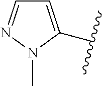 [SMILES: *c1ccnn1C] | Cl | H |
| 2184 | 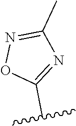 [SMILES: *c1nc(C)no1] | CHF₂ | 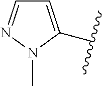 [SMILES: *c1ccnn1C] | Cl | H |
| 2185 | 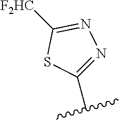 [SMILES: *c1nnc(C(F)F)s1] | CHF₂ | 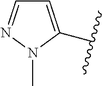 [SMILES: *c1ccnn1C] | Cl | H |
| 2186 | 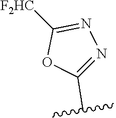 [SMILES: *c1nnc(C(F)F)o1] | CHF₂ | 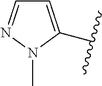 [SMILES: *c1ccnn1C] | Cl | H |
| 2187 | 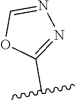 [SMILES: *c1nnco1] | CHF₂ | 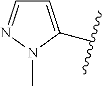 [SMILES: *c1ccnn1C] | Cl | H |
| 2188 | 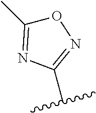 [SMILES: *c1noc(C)n1] | CHF₂ | 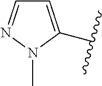 [SMILES: *c1ccnn1C] | Cl | H |
| 2189 | 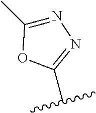 [SMILES: *c1nnc(C)o1] | CF₃ | 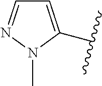 [SMILES: *c1ccnn1C] | Cl | H |
| 2190 | 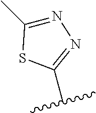 [SMILES: *c1nnc(C)s1] | CF₃ | 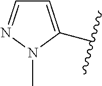 [SMILES: *c1ccnn1C] | Cl | H |
| 2191 | 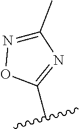 [SMILES: *c1nc(C)no1] | CF₃ | 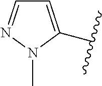 [SMILES: *c1ccnn1C] | Cl | H |
| 2192 | 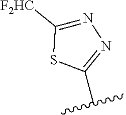 [SMILES: *c1nnc(C(F)F)s1] | CF₃ | 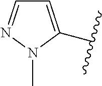 [SMILES: *c1ccnn1C] | Cl | H |
| 2193 | 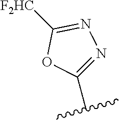 [SMILES: *c1nnc(C(F)F)o1] | CF₃ | 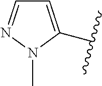 [SMILES: *c1ccnn1C] | Cl | H |
| 2194 | 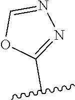 [SMILES: *c1nnco1] | CF₃ | 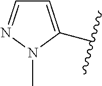 [SMILES: *c1ccnn1C] | Cl | H |
| 2195 | 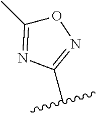 [SMILES: *c1noc(C)n1] | CF₃ | 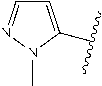 [SMILES: *c1ccnn1C] | Cl | H |
| 2196 | 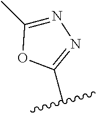 [SMILES: *c1nnc(C)o1] | CHF₂ | 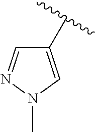 [SMILES: *c1cnn(C)c1] | Cl | H |
| 2197 | 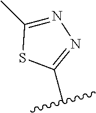 [SMILES: *c1nnc(C)s1] | CHF₂ | 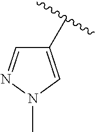 [SMILES: *c1cnn(C)c1] | Cl | H |
| 2198 | 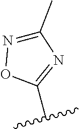 [SMILES: *c1nc(C)no1] | CHF₂ | 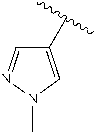 [SMILES: *c1cnn(C)c1] | Cl | H |
| 2199 | 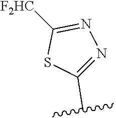 [SMILES: *c1nnc(C(F)F)s1] | CHF₂ | 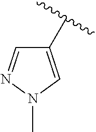 [SMILES: *c1cnn(C)c1] | Cl | H |
| 2200 | 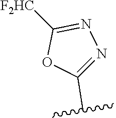 [SMILES: *c1nnc(C(F)F)o1] | CHF₂ | 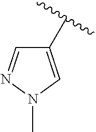 [SMILES: *c1cnn(C)c1] | Cl | H |
| 2201 | 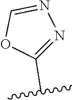 [SMILES: *c1nnco1] | CHF₂ | 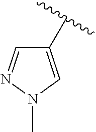 [SMILES: *c1cnn(C)c1] | Cl | H |
| 2202 | 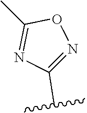 [SMILES: *c1noc(C)n1] | CHF₂ | 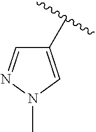 [SMILES: *c1cnn(C)c1] | Cl | H |
| 2203 | 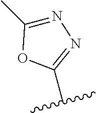 [SMILES: *c1nnc(C)o1] | CF₃ | 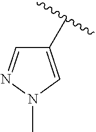 [SMILES: *c1cnn(C)c1] | Cl | H |
| 2204 | 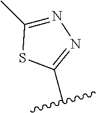 [SMILES: *c1nnc(C)s1] | CF₃ | 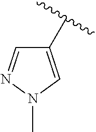 [SMILES: *c1cnn(C)c1] | Cl | H |
| 2205 | 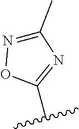 [SMILES: *c1nc(C)no1] | CF₃ | 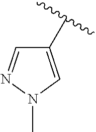 [SMILES: *c1cnn(C)c1] | Cl | H |
| 2206 | 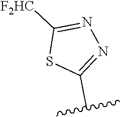 [SMILES: *c1nnc(C(F)F)s1] | CF₃ | 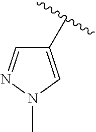 [SMILES: *c1cnn(C)c1] | Cl | H |
| 2207 | 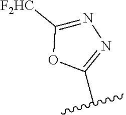 [SMILES: *c1nnc(C(F)F)o1] | CF₃ | 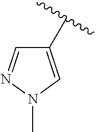 [SMILES: *c1cnn(C)c1] | Cl | H |
| 2208 | 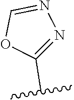 [SMILES: *c1nnco1] | CF₃ | 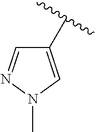 [SMILES: *c1cnn(C)c1] | Cl | H |
| 2209 | 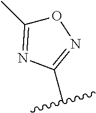 [SMILES: *c1noc(C)n1] | CF₃ | 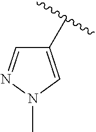 [SMILES: *c1cnn(C)c1] | Cl | H |
| 2210 | 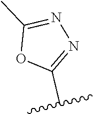 [SMILES: *c1nnc(C)o1] | H | 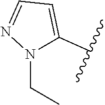 [SMILES: *c1ccnn1CC] | F | H |
| 2211 | 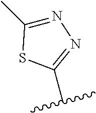 [SMILES: *c1nnc(C)s1] | H | 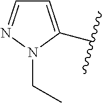 [SMILES: *c1ccnn1CC] | F | H |
| 2212 | 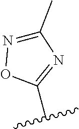 [SMILES: *c1nc(C)no1] | H | 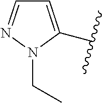 [SMILES: *c1ccnn1CC] | F | H |
| 2213 | 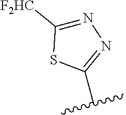 [SMILES: *c1nnc(C(F)F)s1] | H | 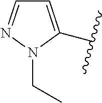 [SMILES: *c1ccnn1CC] | F | H |
| 2214 | 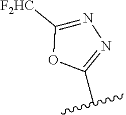 [SMILES: *c1nnc(C(F)F)o1] | H | 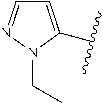 [SMILES: *c1ccnn1CC] | F | H |
| 2215 | 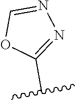 [SMILES: *c1nnco1] | H | 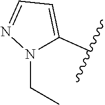 [SMILES: *c1ccnn1CC] | F | H |
| 2216 | 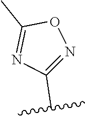 [SMILES: *c1noc(C)n1] | H | 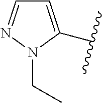 [SMILES: *c1ccnn1CC] | F | H |
| 2217 | 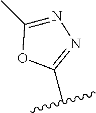 [SMILES: *c1nnc(C)o1] | CH₃ | 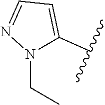 [SMILES: *c1ccnn1CC] | F | H |
| 2218 | 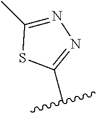 [SMILES: *c1nnc(C)s1] | CH₃ | 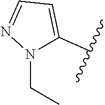 [SMILES: *c1ccnn1CC] | F | H |
| 2219 | 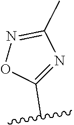 [SMILES: *c1nc(C)no1] | CH₃ | 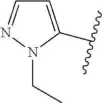 [SMILES: *c1ccnn1CC] | F | H |
| 2220 | 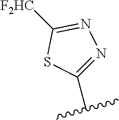 [SMILES: *c1nnc(C(F)F)s1] | CH₃ | 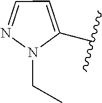 [SMILES: *c1ccnn1CC] | F | H |
| 2221 | 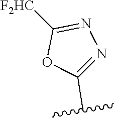 [SMILES: *c1nnc(C(F)F)o1] | CH₃ | 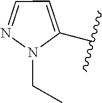 [SMILES: *c1ccnn1CC] | F | H |
| 2222 | 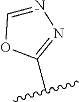 [SMILES: *c1nnco1] | CH₃ | 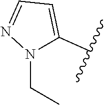 [SMILES: *c1ccnn1CC] | F | H |
| 2223 | 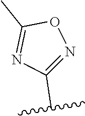 [SMILES: *c1noc(C)n1] | CH₃ | 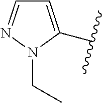 [SMILES: *c1ccnn1CC] | F | H |
| 2224 | 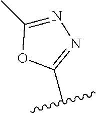 [SMILES: *c1nnc(C)o1] | H | 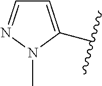 [SMILES: *c1ccnn1C] | F | H |
| 2225 | 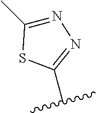 [SMILES: *c1nnc(C)s1] | H | 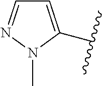 [SMILES: *c1ccnn1C] | F | H |
| 2226 | 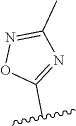 [SMILES: *c1nc(C)no1] | H | 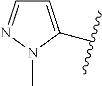 [SMILES: *c1ccnn1C] | F | H |
| 2227 | 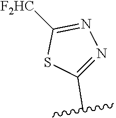 [SMILES: *c1nnc(C(F)F)s1] | H | 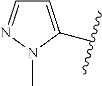 [SMILES: *c1ccnn1C] | F | H |
| 2228 | 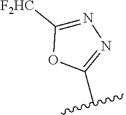 [SMILES: *c1nnc(C(F)F)o1] | H | 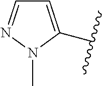 [SMILES: *c1ccnn1C] | F | H |
| 2229 | 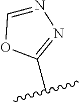 [SMILES: *c1nnco1] | H | 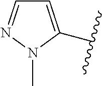 [SMILES: *c1ccnn1C] | F | H |
| 2230 | 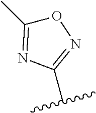 [SMILES: *c1noc(C)n1] | H | 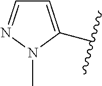 [SMILES: *c1ccnn1C] | F | H |
| 2231 | 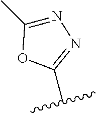 [SMILES: *c1nnc(C)o1] | CH₃ | 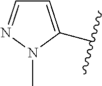 [SMILES: *c1ccnn1C] | F | H |
| 2232 | 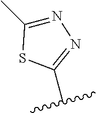 [SMILES: *c1nnc(C)s1] | CH₃ | 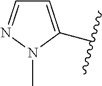 [SMILES: *c1ccnn1C] | F | H |
| 2233 | 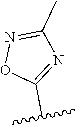 [SMILES: *c1nc(C)no1] | CH₃ | 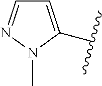 [SMILES: *c1ccnn1C] | F | H |
| 2234 | 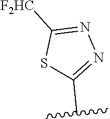 [SMILES: *c1nnc(C(F)F)s1] | CH₃ | 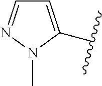 [SMILES: *c1ccnn1C] | F | H |
| 2235 | 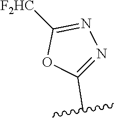 [SMILES: *c1nnc(C(F)F)o1] | CH₃ | 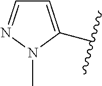 [SMILES: *c1ccnn1C] | F | H |
| 2236 | 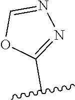 [SMILES: *c1nnco1] | CH₃ | 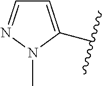 [SMILES: *c1ccnn1C] | F | H |
| 2237 | 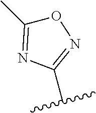 [SMILES: *c1noc(C)n1] | CH₃ | 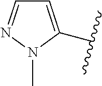 [SMILES: *c1ccnn1C] | F | H |
| 2238 | 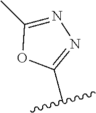 [SMILES: *c1nnc(C)o1] | H | 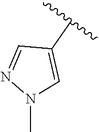 [SMILES: *c1cnn(C)c1] | F | H |
| 2239 | 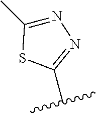 [SMILES: *c1nnc(C)s1] | H | 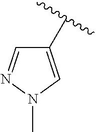 [SMILES: *c1cnn(C)c1] | F | H |
| 2240 | 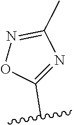 [SMILES: *c1nc(C)no1] | H | 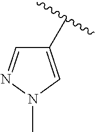 [SMILES: *c1cnn(C)c1] | F | H |
| 2241 | 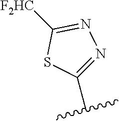 [SMILES: *c1nnc(C(F)F)s1] | H | 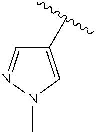 [SMILES: *c1cnn(C)c1] | F | H |
| 2242 | 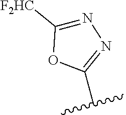 [SMILES: *c1nnc(C(F)F)o1] | H | 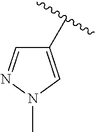 [SMILES: *c1cnn(C)c1] | F | H |
| 2243 | 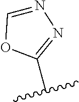 [SMILES: *c1nnco1] | H | 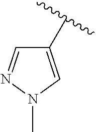 [SMILES: *c1cnn(C)c1] | F | H |
| 2244 | 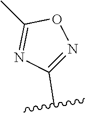 [SMILES: *c1noc(C)n1] | H | 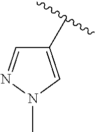 [SMILES: *c1cnn(C)c1] | F | H |
| 2245 | 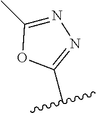 [SMILES: *c1nnc(C)o1] | CH₃ | 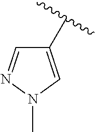 [SMILES: *c1cnn(C)c1] | F | H |
| 2246 | 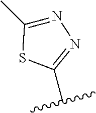 [SMILES: *c1nnc(C)s1] | CH₃ | 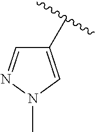 [SMILES: *c1cnn(C)c1] | F | H |
| 2247 | 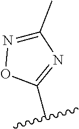 [SMILES: *c1nc(C)no1] | CH₃ | 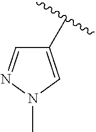 [SMILES: *c1cnn(C)c1] | F | H |
| 2248 | 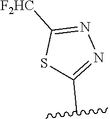 [SMILES: *c1nnc(C(F)F)s1] | CH₃ | 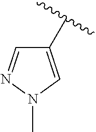 [SMILES: *c1cnn(C)c1] | F | H |
| 2249 | 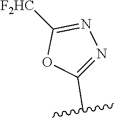 [SMILES: *c1nnc(C(F)F)o1] | CH₃ | 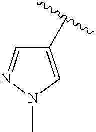 [SMILES: *c1cnn(C)c1] | F | H |
| 2250 | 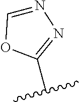 [SMILES: *c1nnco1] | CH₃ | 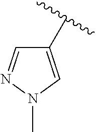 [SMILES: *c1cnn(C)c1] | F | H |
| 2251 | 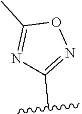 [SMILES: *c1noc(C)n1] | CH₃ | 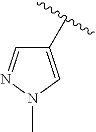 [SMILES: *c1cnn(C)c1] | F | H |
| 2252 | 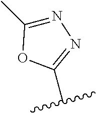 [SMILES: *c1nnc(C)o1] | H | 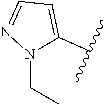 [SMILES: *c1ccnn1CC] | F | F |
| 2253 | 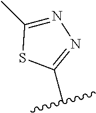 [SMILES: *c1nnc(C)s1] | H | 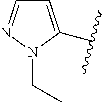 [SMILES: *c1ccnn1CC] | F | F |
| 2254 | 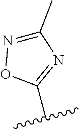 [SMILES: *c1nc(C)no1] | H | 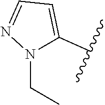 [SMILES: *c1ccnn1CC] | F | F |
| 2255 | 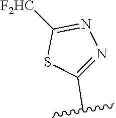 [SMILES: *c1nnc(C(F)F)s1] | H | 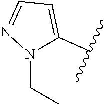 [SMILES: *c1ccnn1CC] | F | F |
| 2256 | 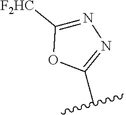 [SMILES: *c1nnc(C(F)F)o1] | H | 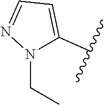 [SMILES: *c1ccnn1CC] | F | F |
| 2257 | 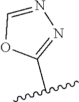 [SMILES: *c1nnco1] | H | 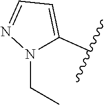 [SMILES: *c1ccnn1CC] | F | F |
| 2258 | 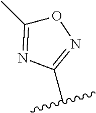 [SMILES: *c1noc(C)n1] | H | 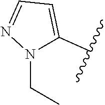 [SMILES: *c1ccnn1CC] | F | F |
| 2259 | 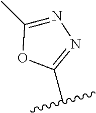 [SMILES: *c1nnc(C)o1] | CH₃ | 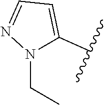 [SMILES: *c1ccnn1CC] | F | F |
| 2260 | 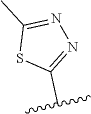 [SMILES: *c1nnc(C)s1] | CH₃ | 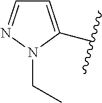 [SMILES: *c1ccnn1CC] | F | F |
| 2261 | 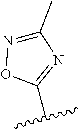 [SMILES: *c1nc(C)no1] | CH₃ | 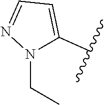 [SMILES: *c1ccnn1CC] | F | F |
| 2262 | 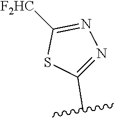 [SMILES: *c1nnc(C(F)F)s1] | CH₃ | 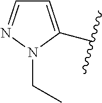 [SMILES: *c1ccnn1CC] | F | F |
| 2263 | 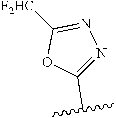 [SMILES: *c1nnc(C(F)F)o1] | CH₃ | 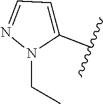 [SMILES: *c1ccnn1CC] | F | F |
| 2264 | 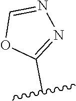 [SMILES: *c1nnco1] | CH₃ | 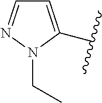 [SMILES: *c1ccnn1CC] | F | F |
| 2265 | 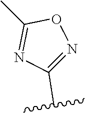 [SMILES: *c1noc(C)n1] | CH₃ | 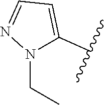 [SMILES: *c1ccnn1CC] | F | F |
| 2266 | 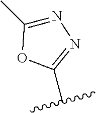 [SMILES: *c1nnc(C)o1] | H | 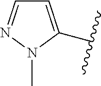 [SMILES: *c1ccnn1C] | F | F |
| 2267 | 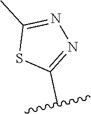 [SMILES: *c1nnc(C)s1] | H | 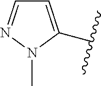 [SMILES: *c1ccnn1C] | F | F |
| 2268 | 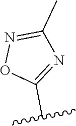 [SMILES: *c1nc(C)no1] | H | 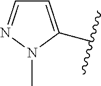 [SMILES: *c1ccnn1C] | F | F |
| 2269 | 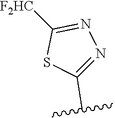 [SMILES: *c1nnc(C(F)F)s1] | H | 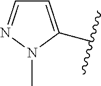 [SMILES: *c1ccnn1C] | F | F |
| 2270 | 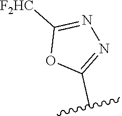 [SMILES: *c1nnc(C(F)F)o1] | H | 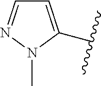 [SMILES: *c1ccnn1C] | F | F |
| 2271 | 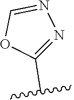 [SMILES: *c1nnco1] | H | 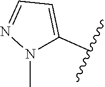 [SMILES: *c1ccnn1C] | F | F |
| 2272 | 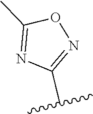 [SMILES: *c1noc(C)n1] | H | 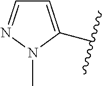 [SMILES: *c1ccnn1C] | F | F |
| 2273 | 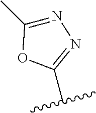 [SMILES: *c1nnc(C)o1] | CH₃ | 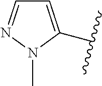 [SMILES: *c1ccnn1C] | F | F |
| 2274 | 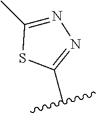 [SMILES: *c1nnc(C)s1] | CH₃ | 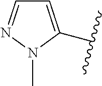 [SMILES: *c1ccnn1C] | F | F |
| 2275 | 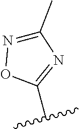 [SMILES: *c1nc(C)no1] | CH₃ | 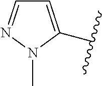 [SMILES: *c1ccnn1C] | F | F |
| 2276 | 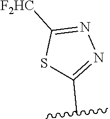 [SMILES: *c1nnc(C(F)F)s1] | CH₃ | 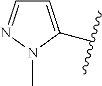 [SMILES: *c1ccnn1C] | F | F |
| 2277 | 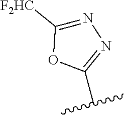 [SMILES: *c1nnc(C(F)F)o1] | CH₃ | 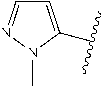 [SMILES: *c1ccnn1C] | F | F |
| 2278 | 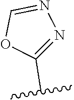 [SMILES: *c1nnco1] | CH₃ | 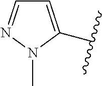 [SMILES: *c1ccnn1C] | F | F |
| 2279 | 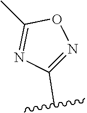 [SMILES: *c1noc(C)n1] | CH₃ | 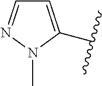 [SMILES: *c1ccnn1C] | F | F |
| 2280 | 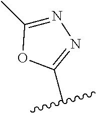 [SMILES: *c1nnc(C)o1] | H | 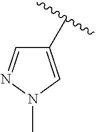 [SMILES: *c1cnn(C)c1] | F | F |
| 2281 | 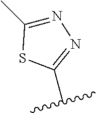 [SMILES: *c1nnc(C)s1] | H | 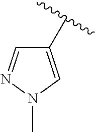 [SMILES: *c1cnn(C)c1] | F | F |
| 2282 | 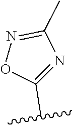 [SMILES: *c1nc(C)no1] | H | 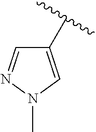 [SMILES: *c1cnn(C)c1] | F | F |
| 2283 | 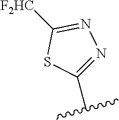 [SMILES: *c1nnc(C(F)F)s1] | H | 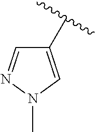 [SMILES: *c1cnn(C)c1] | F | F |
| 2284 | 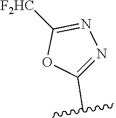 [SMILES: *c1nnc(C(F)F)o1] | H | 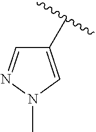 [SMILES: *c1cnn(C)c1] | F | F |
| 2285 | 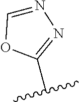 [SMILES: *c1nnco1] | H | 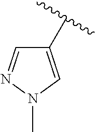 [SMILES: *c1cnn(C)c1] | F | F |
| 2286 | 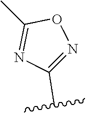 [SMILES: *c1noc(C)n1] | H | 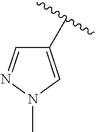 [SMILES: *c1cnn(C)c1] | F | F |
| 2287 | 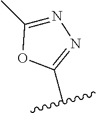 [SMILES: *c1nnc(C)o1] | CH₃ | 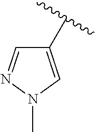 [SMILES: *c1cnn(C)c1] | F | F |
| 2288 | 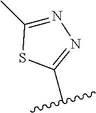 [SMILES: *c1nnc(C)s1] | CH₃ | 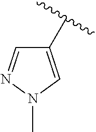 [SMILES: *c1cnn(C)c1] | F | F |
| 2289 | 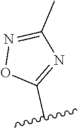 [SMILES: *c1nc(C)no1] | CH₃ | 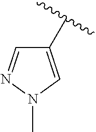 [SMILES: *c1cnn(C)c1] | F | F |
| 2290 | 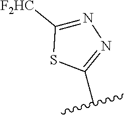 [SMILES: *c1nnc(C(F)F)s1] | CH₃ | 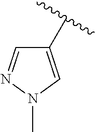 [SMILES: *c1cnn(C)c1] | F | F |
| 2291 | 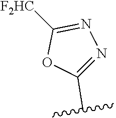 [SMILES: *c1nnc(C(F)F)o1] | CH₃ | 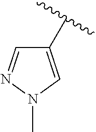 [SMILES: *c1cnn(C)c1] | F | F |
| 2292 | 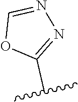 [SMILES: *c1nnco1] | CH₃ | 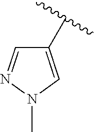 [SMILES: *c1cnn(C)c1] | F | F |
| 2293 | 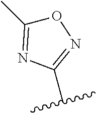 [SMILES: *c1noc(C)n1] | CH₃ | 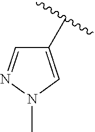 [SMILES: *c1cnn(C)c1] | F | F |
| 2294 | 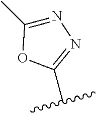 [SMILES: *c1nnc(C)o1] | H | 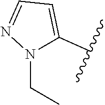 [SMILES: *c1ccnn1CC] | Cl | H |
| 2295 | 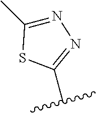 [SMILES: *c1nnc(C)s1] | H | 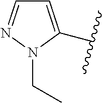 [SMILES: *c1ccnn1CC] | Cl | H |
| 2296 | 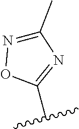 [SMILES: *c1nc(C)no1] | H | 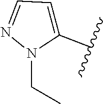 [SMILES: *c1ccnn1CC] | Cl | H |
| 2297 | 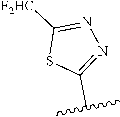 [SMILES: *c1nnc(C(F)F)s1] | H | 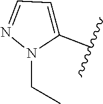 [SMILES: *c1ccnn1CC] | Cl | H |
| 2298 | 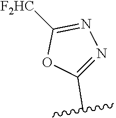 [SMILES: *c1nnc(C(F)F)o1] | H | 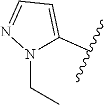 [SMILES: *c1ccnn1CC] | Cl | H |
| 2299 | 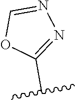 [SMILES: *c1nnco1] | H | 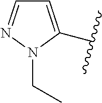 [SMILES: *c1ccnn1CC] | Cl | H |
| 2300 | 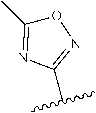 [SMILES: *c1noc(C)n1] | H | 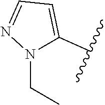 [SMILES: *c1ccnn1CC] | Cl | H |
| 2301 | 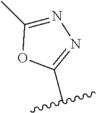 [SMILES: *c1nnc(C)o1] | CH₃ | 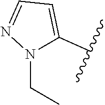 [SMILES: *c1ccnn1CC] | Cl | H |
| 2302 | 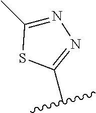 [SMILES: *c1nnc(C)s1] | CH₃ | 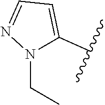 [SMILES: *c1ccnn1CC] | Cl | H |
| 2303 | 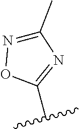 [SMILES: *c1nc(C)no1] | CH₃ | 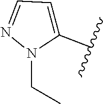 [SMILES: *c1ccnn1CC] | Cl | H |
| 2304 | 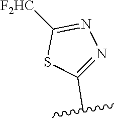 [SMILES: *c1nnc(C(F)F)s1] | CH₃ | 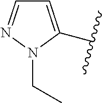 [SMILES: *c1ccnn1CC] | Cl | H |
| 2305 | 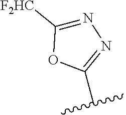 [SMILES: *c1nnc(C(F)F)o1] | CH₃ | 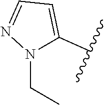 [SMILES: *c1ccnn1CC] | Cl | H |
| 2306 | 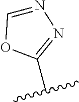 [SMILES: *c1nnco1] | CH₃ | 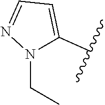 [SMILES: *c1ccnn1CC] | Cl | H |
| 2307 | 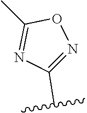 [SMILES: *c1noc(C)n1] | CH | 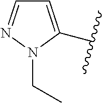 [SMILES: *c1ccnn1CC] | Cl | H |
| 2308 | 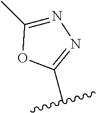 [SMILES: *c1nnc(C)o1] | H | 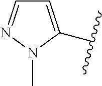 [SMILES: *c1ccnn1C] | Cl | H |
| 2309 | 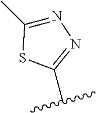 [SMILES: *c1nnc(C)s1] | H | 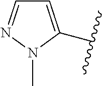 [SMILES: *c1ccnn1C] | Cl | H |
| 2310 | 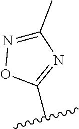 [SMILES: *c1nc(C)no1] | H | 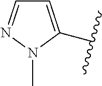 [SMILES: *c1ccnn1C] | Cl | H |
| 2311 | 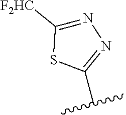 [SMILES: *c1nnc(C(F)F)s1] | H | 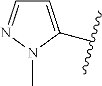 [SMILES: *c1ccnn1C] | Cl | H |
| 2312 | 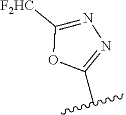 [SMILES: *c1nnc(C(F)F)o1] | H | 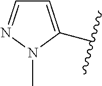 [SMILES: *c1ccnn1C] | Cl | H |
| 2313 | 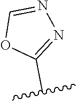 [SMILES: *c1nnco1] | H | 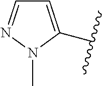 [SMILES: *c1ccnn1C] | Cl | H |
| 2314 | 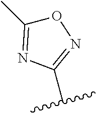 [SMILES: *c1noc(C)n1] | H | 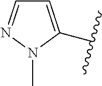 [SMILES: *c1ccnn1C] | Cl | H |
| 2315 | 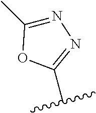 [SMILES: *c1nnc(C)o1] | CH₃ | 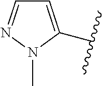 [SMILES: *c1ccnn1C] | Cl | H |
| 2316 | 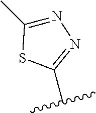 [SMILES: *c1nnc(C)s1] | CH₃ | 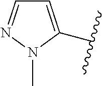 [SMILES: *c1ccnn1C] | Cl | H |
| 2317 | 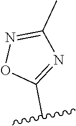 [SMILES: *c1nc(C)no1] | CH₃ | 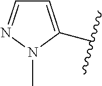 [SMILES: *c1ccnn1C] | Cl | H |
| 2317 | 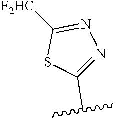 [SMILES: *c1nnc(C(F)F)s1] | CH₃ | 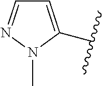 [SMILES: *c1ccnn1C] | Cl | H |
| 2319 | 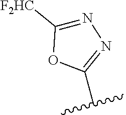 [SMILES: *c1nnc(C(F)F)o1] | CH₃ | 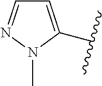 [SMILES: *c1ccnn1C] | Cl | H |
| 2320 | 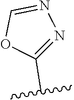 [SMILES: *c1nnco1] | CH₃ | 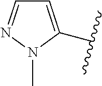 [SMILES: *c1ccnn1C] | Cl | H |
| 2321 | 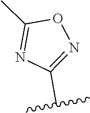 [SMILES: *c1noc(C)n1] | CH₃ | 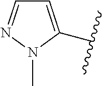 [SMILES: *c1ccnn1C] | Cl | H |
| 2322 | 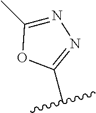 [SMILES: *c1nnc(C)o1] | H | 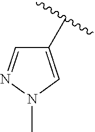 [SMILES: *c1cnn(C)c1] | Cl | H |
| 2323 | 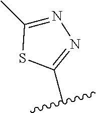 [SMILES: *c1nnc(C)s1] | H | 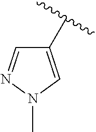 [SMILES: *c1cnn(C)c1] | Cl | H |
| 2324 | 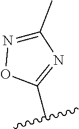 [SMILES: *c1nc(C)no1] | H | 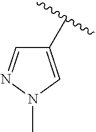 [SMILES: *c1cnn(C)c1] | Cl | H |
| 2325 | 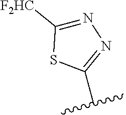 [SMILES: *c1nnc(C(F)F)s1] | H | 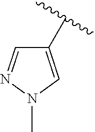 [SMILES: *c1cnn(C)c1] | Cl | H |
| 2326 | 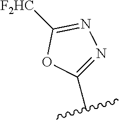 [SMILES: *c1nnc(C(F)F)o1] | H | 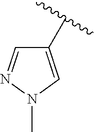 [SMILES: *c1cnn(C)c1] | Cl | H |
| 2327 | 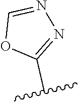 [SMILES: *c1nnco1] | H | 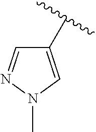 [SMILES: *c1cnn(C)c1] | Cl | H |
| 2328 | 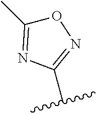 [SMILES: *c1noc(C)n1] | H | 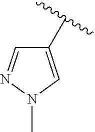 [SMILES: *c1cnn(C)c1] | Cl | H |
| 2329 | 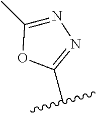 [SMILES: *c1nnc(C)o1] | CH₃ | 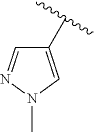 [SMILES: *c1cnn(C)c1] | Cl | H |
| 2330 | 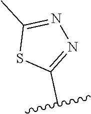 [SMILES: *c1nnc(C)s1] | CH₃ | 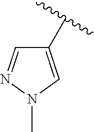 [SMILES: *c1cnn(C)c1] | Cl | H |
| 2331 | 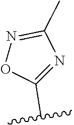 [SMILES: *c1nc(C)no1] | CH₃ | 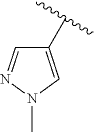 [SMILES: *c1cnn(C)c1] | Cl | H |
| 2334 | 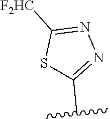 [SMILES: *c1nnc(C(F)F)s1] | CH₃ | 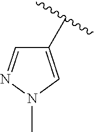 [SMILES: *c1cnn(C)c1] | Cl | H |
| 2335 | 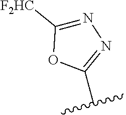 [SMILES: *c1nnc(C(F)F)o1] | CH₃ | 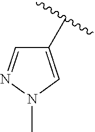 [SMILES: *c1cnn(C)c1] | Cl | H |
| 2336 | 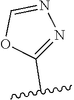 [SMILES: *c1nnco1] | CH₃ | 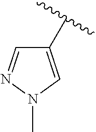 [SMILES: *c1cnn(C)c1] | Cl | H |
| 2337 | 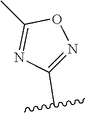 [SMILES: *c1noc(C)n1] | CH₃ | 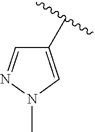 [SMILES: *c1cnn(C)c1] | Cl | H |

In some embodiments, the compound of formula (A), formula (I), or formula (II) is a compound having any one of the following formula, or a pharmaceutically acceptable salt, solvate, hydrate, cocrystal, or prodrug thereof:

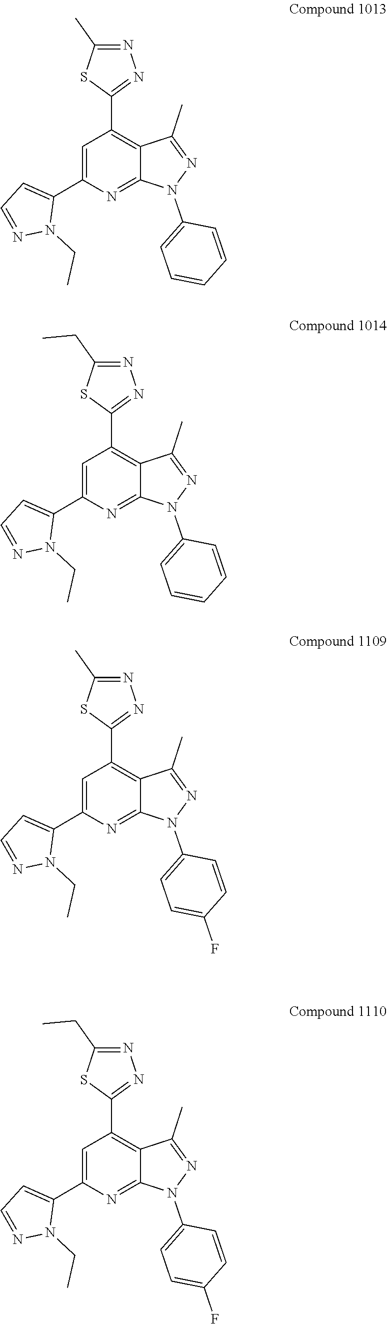 [SMILES: CCn1nccc1-c1cc(-c2nnc(C)s2)c2c(C)nn(-c3ccccc3)c2n1 ; CCc1nnc(-c2cc(-c3ccnn3CC)nc3c2c(C)nn3-c2ccccc2)s1 ; CCn1nccc1-c1cc(-c2nnc(C)s2)c2c(C)nn(-c3ccc(F)cc3)c2n1 ; CCc1nnc(-c2cc(-c3ccnn3CC)nc3c2c(C)nn3-c2ccc(F)cc2)s1]

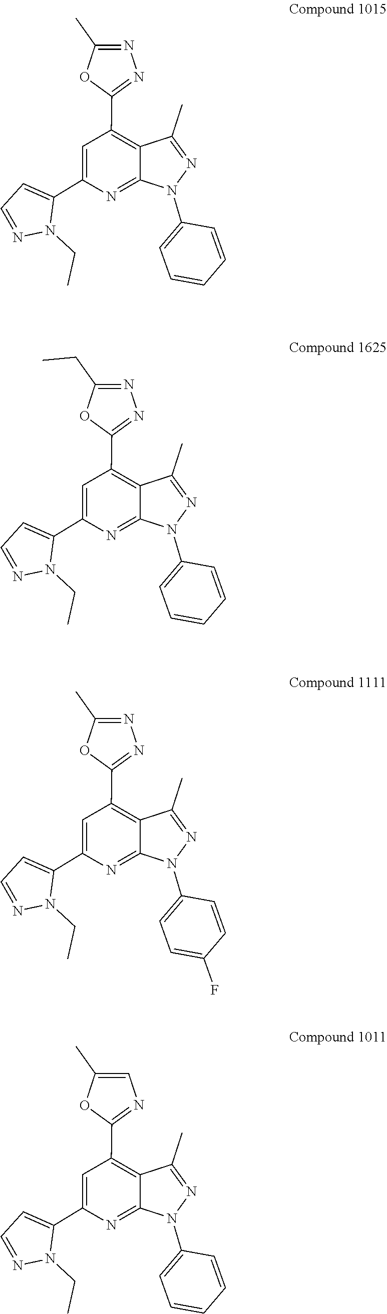 [SMILES: CCn1nccc1-c1cc(-c2nnc(C)o2)c2c(C)nn(-c3ccccc3)c2n1 ; CCc1nnc(-c2cc(-c3ccnn3CC)nc3c2c(C)nn3-c2ccccc2)o1 ; CCn1nccc1-c1cc(-c2nnc(C)o2)c2c(C)nn(-c3ccc(F)cc3)c2n1 ; CCn1nccc1-c1cc(-c2ncc(C)o2)c2c(C)nn(-c3ccccc3)c2n1]

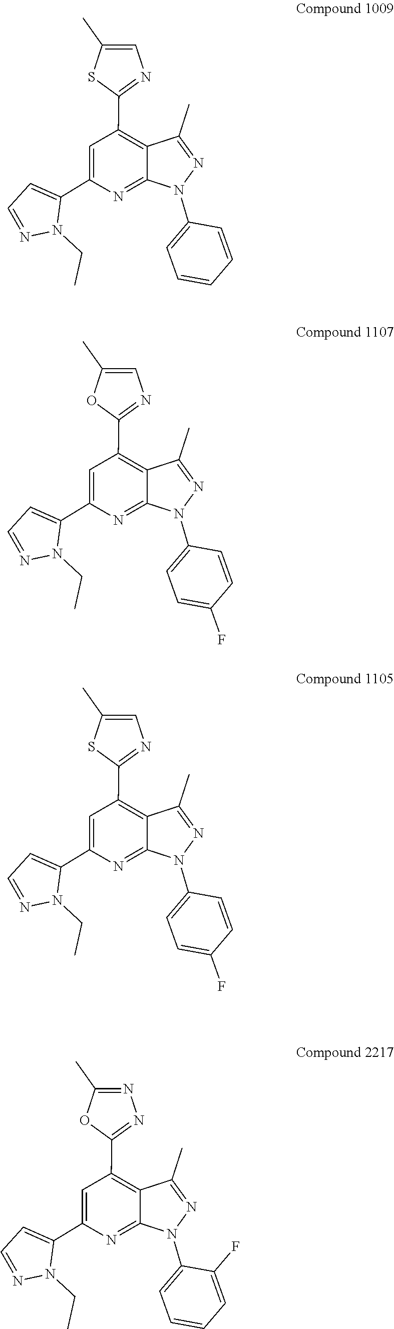 [SMILES: CCn1nccc1-c1cc(-c2ncc(C)s2)c2c(C)nn(-c3ccccc3)c2n1 ; CCn1nccc1-c1cc(-c2ncc(C)o2)c2c(C)nn(-c3ccc(F)cc3)c2n1 ; CCn1nccc1-c1cc(-c2ncc(C)s2)c2c(C)nn(-c3ccc(F)cc3)c2n1 ; CCn1nccc1-c1cc(-c2nnc(C)o2)c2c(C)nn(-c3ccccc3F)c2n1]

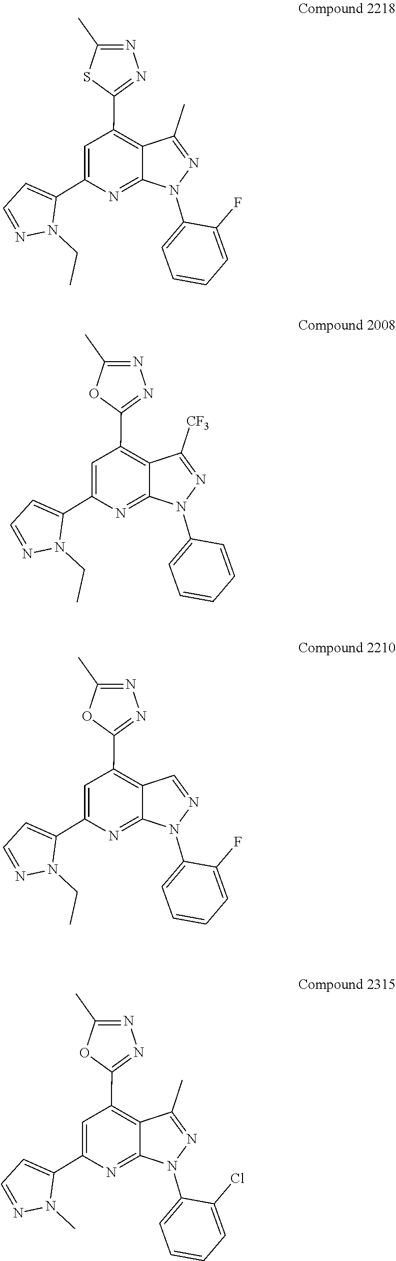 [SMILES: CCn1nccc1-c1cc(-c2nnc(C)s2)c2c(C)nn(-c3ccccc3F)c2n1 ; CCn1nccc1-c1cc(-c2nnc(C)o2)c2c(C(F)(F)F)nn(-c3ccccc3)c2n1 ; CCn1nccc1-c1cc(-c2nnc(C)o2)c2cnn(-c3ccccc3F)c2n1 ; Cc1nnc(-c2cc(-c3ccnn3C)nc3c2c(C)nn3-c2ccccc2Cl)o1]

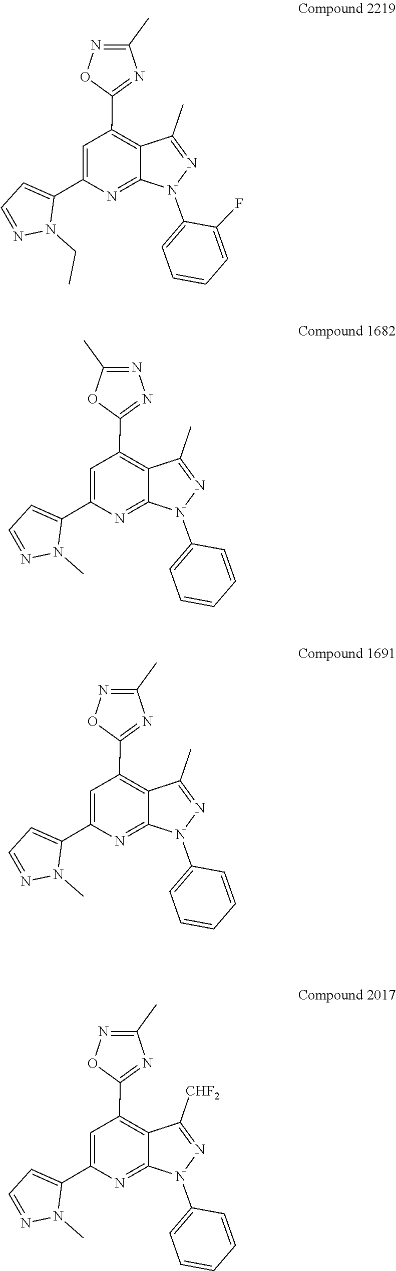 [SMILES: CCn1nccc1-c1cc(-c2nc(C)no2)c2c(C)nn(-c3ccccc3F)c2n1 ; Cc1nnc(-c2cc(-c3ccnn3C)nc3c2c(C)nn3-c2ccccc2)o1 ; Cc1noc(-c2cc(-c3ccnn3C)nc3c2c(C)nn3-c2ccccc2)n1 ; Cc1noc(-c2cc(-c3ccnn3C)nc3c2c(C(F)F)nn3-c2ccccc2)n1]

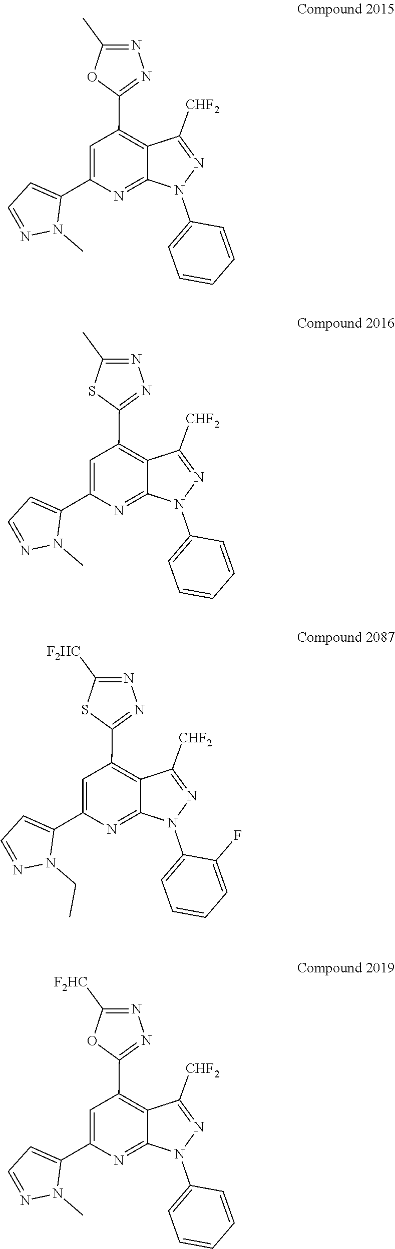 [SMILES: Cc1nnc(-c2cc(-c3ccnn3C)nc3c2c(C(F)F)nn3-c2ccccc2)o1 ; Cc1nnc(-c2cc(-c3ccnn3C)nc3c2c(C(F)F)nn3-c2ccccc2)s1 ; CCn1nccc1-c1cc(-c2nnc(C(F)F)s2)c2c(C(F)F)nn(-c3ccccc3F)c2n1 ; Cn1nccc1-c1cc(-c2nnc(C(F)F)o2)c2c(C(F)F)nn(-c3ccccc3)c2n1]

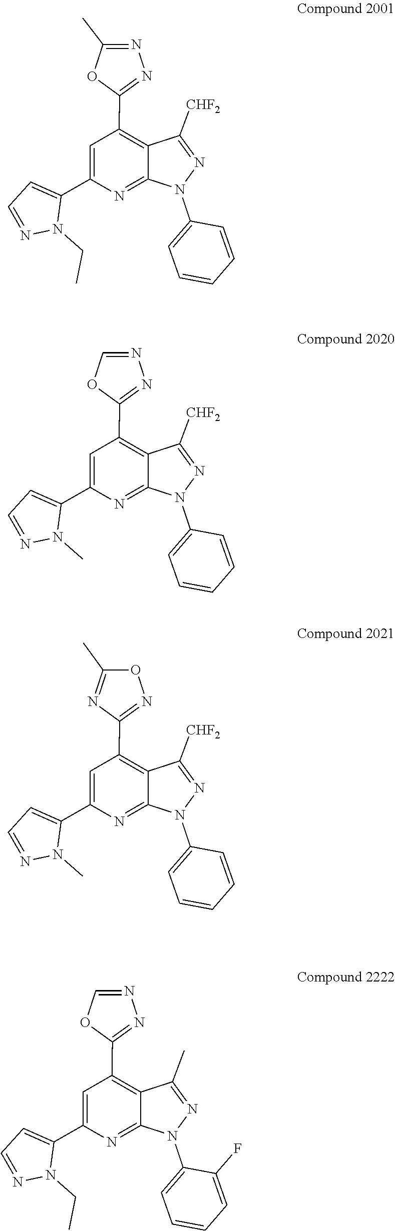 [SMILES: CCn1nccc1-c1cc(-c2nnc(C)o2)c2c(C(F)F)nn(-c3ccccc3)c2n1 ; Cn1nccc1-c1cc(-c2nnco2)c2c(C(F)F)nn(-c3ccccc3)c2n1 ; Cc1nc(-c2cc(-c3ccnn3C)nc3c2c(C(F)F)nn3-c2ccccc2)no1 ; CCn1nccc1-c1cc(-c2nnco2)c2c(C)nn(-c3ccccc3F)c2n1]

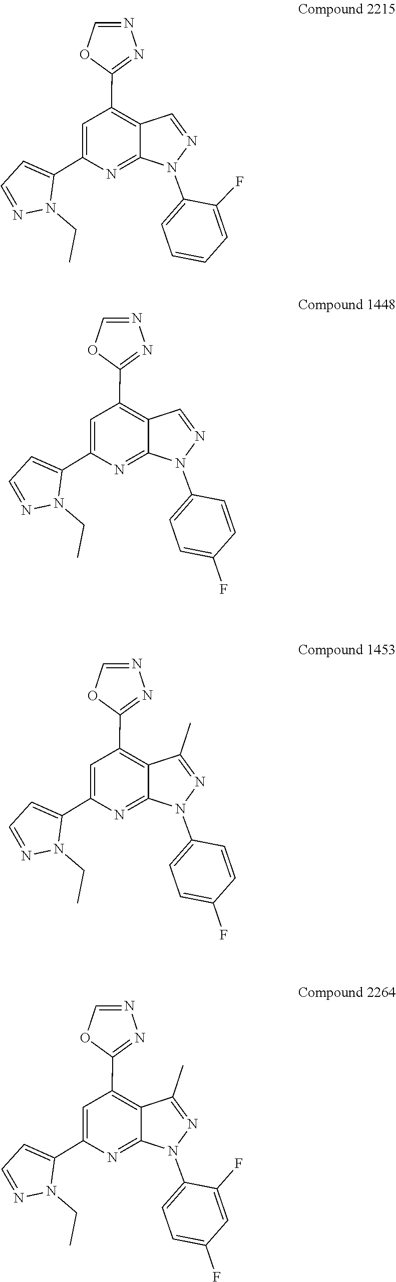 [SMILES: CCn1nccc1-c1cc(-c2nnco2)c2cnn(-c3ccccc3F)c2n1 ; CCn1nccc1-c1cc(-c2nnco2)c2cnn(-c3ccc(F)cc3)c2n1 ; CCn1nccc1-c1cc(-c2nnco2)c2c(C)nn(-c3ccc(F)cc3)c2n1 ; CCn1nccc1-c1cc(-c2nnco2)c2c(C)nn(-c3ccc(F)cc3F)c2n1]

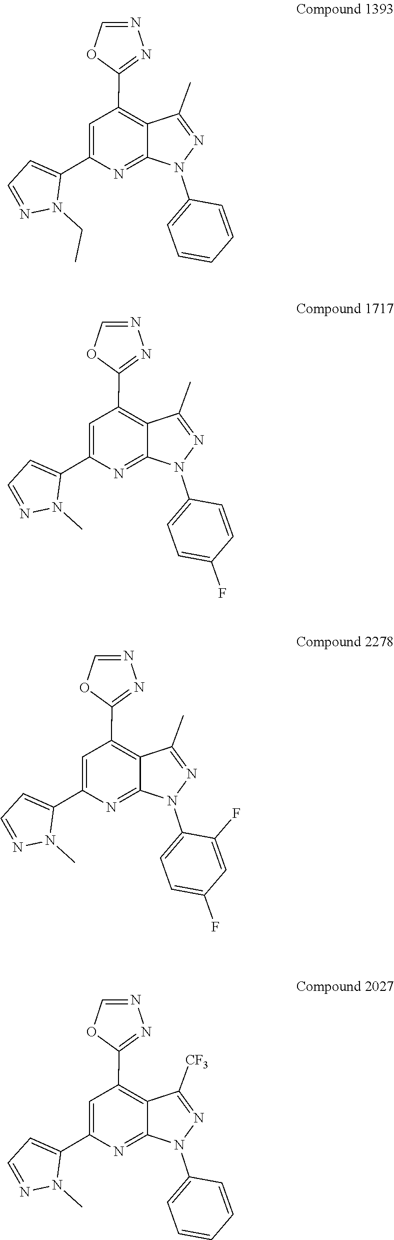 [SMILES: CCn1nccc1-c1cc(-c2nnco2)c2c(C)nn(-c3ccccc3)c2n1 ; Cc1nn(-c2ccc(F)cc2)c2nc(-c3ccnn3C)cc(-c3nnco3)c12 ; Cc1nn(-c2ccc(F)cc2F)c2nc(-c3ccnn3C)cc(-c3nnco3)c12 ; Cn1nccc1-c1cc(-c2nnco2)c2c(C(F)(F)F)nn(-c3ccccc3)c2n1]

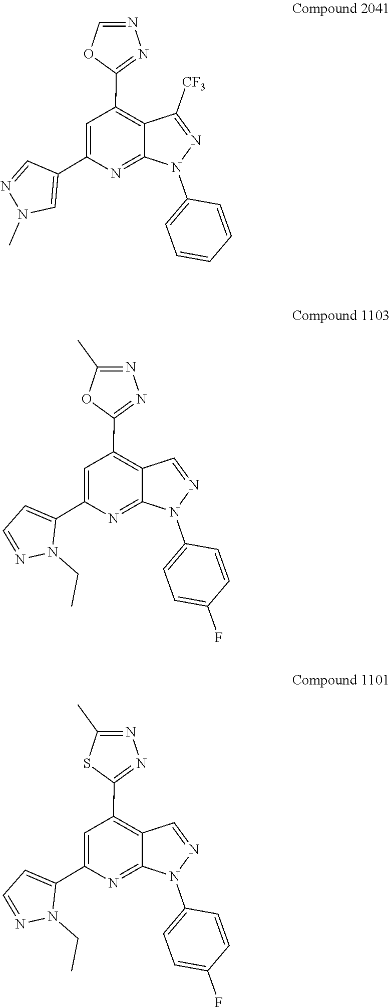 [SMILES: Cn1cc(-c2cc(-c3nnco3)c3c(C(F)(F)F)nn(-c4ccccc4)c3n2)cn1 ; CCn1nccc1-c1cc(-c2nnc(C)o2)c2cnn(-c3ccc(F)cc3)c2n1 ; CCn1nccc1-c1cc(-c2nnc(C)s2)c2cnn(-c3ccc(F)cc3)c2n1]

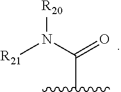

In some embodiments, R¹ in formula (A) is.

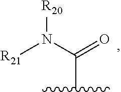

In some embodiments, R¹ in formula (A) is, R₂₀ is selected from H, optionally substituted C₁-C₆ alkyl, and optionally substituted C₁-C₆ haloalkyl; R₂₁ is selected from optionally substituted C₁-C₆ alkyl and optionally substituted C₁-C₆ haloalkyl; or R₂₀ and R₂₁ are joined to form a 4-6-membered heterocycle with the nitrogen to which they are each bound, wherein the heterocycle may comprise 1 additional heteroatom selected from O and NH.

In some embodiments, R₂₀ is selected from H, methyl, ethyl, propyl, isopropyl, butyl, tert-butyl, CF₃, CHF₂, CH₂F, CH₂CF₃, CH₂CHF₂, and CH₂CH₂F; and R₂₁ is selected from methyl, ethyl, propyl, isopropyl, butyl, tert-butyl, CF₃, CHF₂, CH₂F, CH₂CF₃, CH₂CHF₂, and CH₂CH₂F; or R₂₀ and R₂₁ are joined to form a 4-6-membered heterocycle with the nitrogen to which they are each bound, wherein the heterocycle is selected from azetidine, pyrrolidine, morpholine, and piperazine.

In some embodiments, R₂₀ is selected from H, methyl, ethyl, propyl, tert-butyl, and CH₂CH₂F; and R₂₁ is selected from methyl, ethyl, propyl, tert-butyl, and CH₂CH₂F; or R₂₀ and R₂₁ are joined to form a 4-6-membered heterocycle with the nitrogen to which they are each bound, wherein the heterocycle is selected from azetidine, pyrrolidine, morpholine, and piperazine.

In some embodiments, the compound of formula (A) is a compound having formula (III), or a pharmaceutically acceptable salt, solvate, hydrate, cocrystal, or prodrug thereof: or pharmaceutically acceptable salt, solvate, hydrate, cocrystal, or prodrug thereof:

wherein in formula (III):

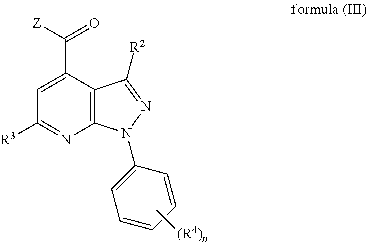

Z is selected from

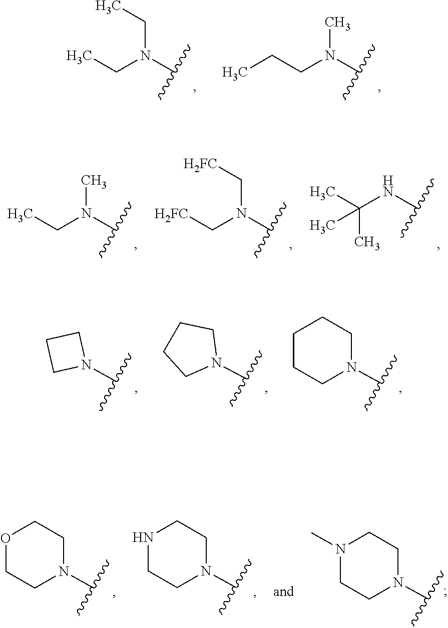

R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

In some embodiments, R₂ is selected from hydrogen, C₃-C₆ cycloalkyl, C₁-C₆ alkyl (e.g., C₁-C₄ straight chain alkyl, C₃-C₆ branched alkyl), —CF₃, —CH₂F, —CHF₂, and —C(R⁶R⁷)ₚX; wherein, p is an integer from 1-3; R⁶ and R⁷ are each independently selected from hydrogen, C₁-C₃ alkyl, and C₃-C₆ cycloalkyl; X is selected from —OH, —CN, —OR⁸, and NR⁹R¹⁰; R⁸ is selected from C₁-C₃ alkyl (e.g., C₁-C₃ straight or branched alkyl), and C₃-C₆ cycloalkyl; and R⁹ and R¹⁰ are each independently selected from hydrogen, C₁-C₃ alkyl, and C₃-C₆ cycloalkyl.

In some embodiments, R₂ is selected from hydrogen, C₁-C₃ alkyl (e.g., methyl, ethyl, propyl, isopropyl). —CF₃, —CH₂F, and —CHF₂.

In some embodiments, R₂ is selected from hydrogen and —CH₃.

In some embodiments, R³ is a 5-membered heteroaryl ring, optionally selected from pyrrole, thiophene, furan, pyrazole, thiazole, isothiazole, oxazole, isoxazole, and imidazole.

In some embodiments, R³ is selected from

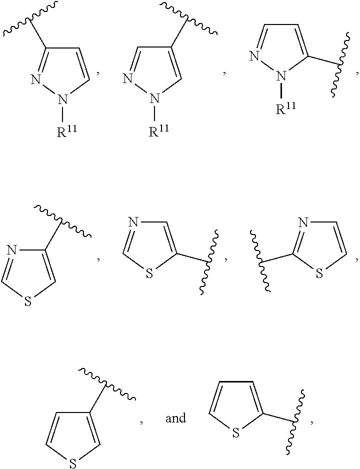

wherein R¹¹ is selected from hydrogen, C₁-C₃ alkyl (e.g., methyl, ethyl, propyl, isopropyl), C₃-C₆ cycloalkyl, CF₃, CH₂CF₃, CH₂CHF₂, and CH₂CH₂F.

In some embodiments, R³ is selected from

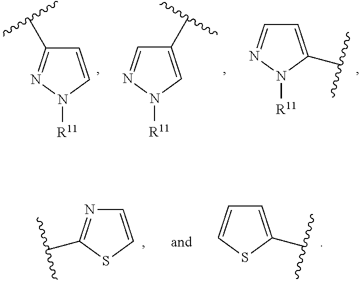

In some embodiments, R¹¹ is selected from hydrogen, methyl, ethyl, propyl, isopropyl, —CF₃, and —CH₂CF₃.

In some embodiments, R³ is selected from

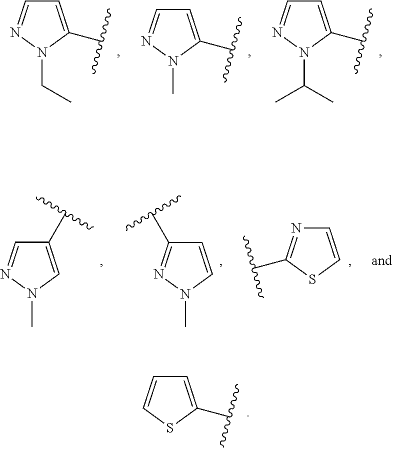

In some embodiments, R⁴ is selected from hydrogen, C₁-C₆ alkyl, and halo (e.g., F or Cl). In some embodiments, n is 0 or 1.

In some embodiments, R⁴ is selected from hydrogen, 4-fluoro, 3-fluoro, 2-fluoro, 2-chloro, and 4-chloro. In some embodiments, R⁴ is selected from hydrogen, 4-fluoro, 3-fluoro, 2-fluoro, and 4-chloro.

In some embodiments, the compound of of formula (A) or formula (III) is a compound having any one of formula 3001-3031, or a pharmaceutically acceptable salt, solvate, hydrate, cocrystal, or prodrug thereof:

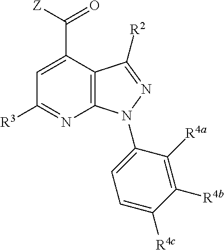

| Cpd. # | Z | R² | R³ | R^{4a} | R^{4b} | R^{4c} |
|---|---|---|---|---|---|---|
| 3001 | 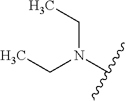 [SMILES: *N(CC)CC] | CH₃ | 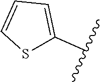 [SMILES: *c1cccs1] | H | H | H |
| 3002 | 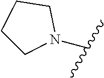 [SMILES: *N1CCCC1] | CH₃ | 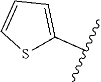 [SMILES: *c1cccs1] | H | H | H |
| 3003 | 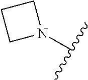 [SMILES: *N1CCC1] | CH₃ | 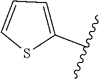 [SMILES: *c1cccs1] | H | H | H |
| 3004 | 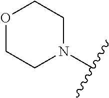 [SMILES: *N1CCOCC1] | CH₃ | 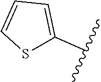 [SMILES: *c1cccs1] | H | H | H |
| 3005 | 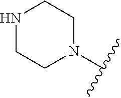 [SMILES: *N1CCNCC1] | CH₃ | 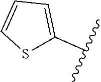 [SMILES: *c1cccs1] | H | H | H |
| 3006 | 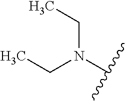 [SMILES: *N(CC)CC] | CH₃ | 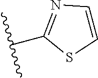 [SMILES: *c1nccs1] | H | H | H |
| 3007 | 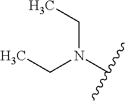 [SMILES: *N(CC)CC] | CH₃ | 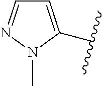 [SMILES: *c1ccnn1C] | H | H | H |
| 3008 | 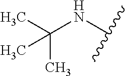 [SMILES: *NC(C)(C)C] | CH₃ | 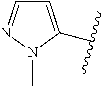 [SMILES: *c1ccnn1C] | H | H | H |
| 3009 | 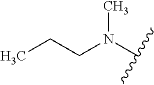 [SMILES: *N(C)CCC] | CH₃ | 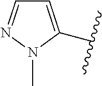 [SMILES: *c1ccnn1C] | H | H | H |
| 3010 | 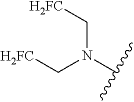 | CH₃ | 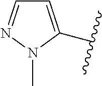 [SMILES: *c1ccnn1C] | H | H | H |
| 3011 | 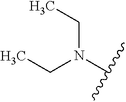 [SMILES: *N(CC)CC] | CH₃ | 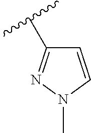 [SMILES: *c1ccn(C)n1] | H | H | H |
| 3012 | 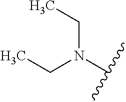 [SMILES: *N(CC)CC] | CH₃ | 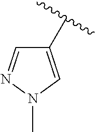 [SMILES: *c1cnn(C)c1] | H | H | H |
| 3013 | 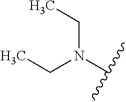 [SMILES: *N(CC)CC] | CH₃ | 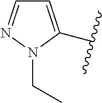 [SMILES: *c1ccnn1CC] | H | H | H |
| 3014 | 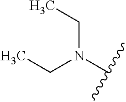 [SMILES: *N(CC)CC] | CH₃ | 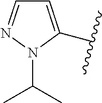 [SMILES: *c1ccnn1C(C)C] | H | H | H |
| 3015 | 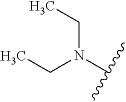 [SMILES: *N(CC)CC] | CH₃ | 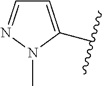 [SMILES: *c1ccnn1C] | H | H | F |
| 3016 | 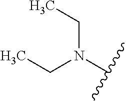 [SMILES: *N(CC)CC] | CH₃ | 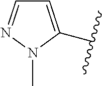 [SMILES: *c1ccnn1C] | H | F | H |
| 3017 | 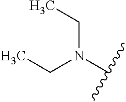 [SMILES: *N(CC)CC] | CH₃ | 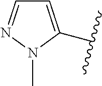 [SMILES: *c1ccnn1C] | H | H | Cl |
| 3018 | 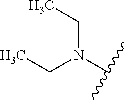 [SMILES: *N(CC)CC] | CH₃ | 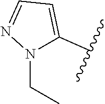 [SMILES: *c1ccnn1CC] | H | H | F |
| 3019 | 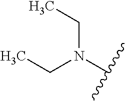 [SMILES: *N(CC)CC] | CH₃ | 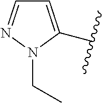 [SMILES: *c1ccnn1CC] | F | H | H |
| 3020 | 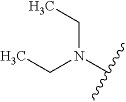 [SMILES: *N(CC)CC] | H | 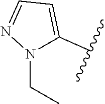 [SMILES: *c1ccnn1CC] | H | H | H |
| 3021 | 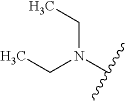 [SMILES: *N(CC)CC] | H | 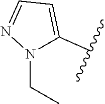 [SMILES: *c1ccnn1CC] | H | H | F |
| 3022 | 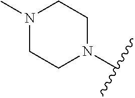 [SMILES: *N1CCN(C)CC1] | CH₃ | 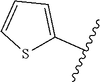 [SMILES: *c1cccs1] | H | H | H |
| 3023 | 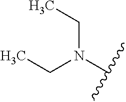 [SMILES: *N(CC)CC] | CH₃ | 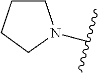 [SMILES: *N1CCCC1] | H | H | H |
| 3024 | 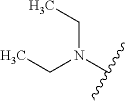 [SMILES: *N(CC)CC] | CH₃ | 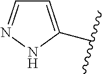 [SMILES: *c1ccn[nH]1] | H | H | H |
| 3025 | 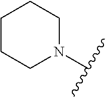 [SMILES: *N1CCCCC1] | CH₃ | 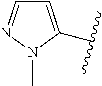 [SMILES: *c1ccnn1C] | H | H | H |
| 3026 | 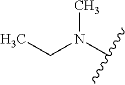 [SMILES: *N(C)CC] | CH₃ | 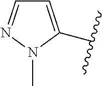 [SMILES: *c1ccnn1C] | H | H | H |
| 3027 | 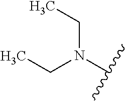 [SMILES: *N(CC)CC] | CH₃ | 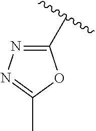 [SMILES: *c1nnc(C)o1] | H | H | H |
| 3028 | 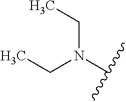 [SMILES: *N(CC)CC] | CH₃ | 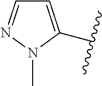 [SMILES: *c1ccnn1C] | Cl | H | H |
| 3029 | 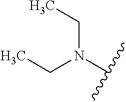 [SMILES: *N(CC)CC] | CF₃ | 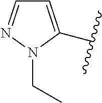 [SMILES: *c1ccnn1CC] | H | H | H |
| 3030 | 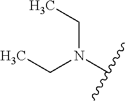 [SMILES: *N(CC)CC] | CHF₂ | 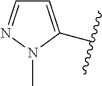 [SMILES: *c1ccnn1C] | H | H | H |
| 3031 | 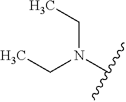 [SMILES: *N(CC)CC] | CH₃ | 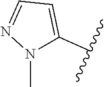 [SMILES: *c1ccnn1C] | H | H | H |

In some embodiments, the compound having a formula of any one of formula (A), formula (I), formula (II), formula (III), formula 1001-1729, formula 2001-2337, formula 3001-3031, or formula 4001-4729, or a pharmaceutically acceptable salt, solvate, hydrate, cocrystal, or prodrug thereof, is a PDE11A4 inhibitor.

### Methods of Treatment

The compounds and compositions described herein can be used in methods for treating diseases and/or disorders. In some embodiments, the compounds and compositions described herein can be used in methods for treating diseases associated with PDE11A activity. In some embodiments, the compounds and compositions described herein can be used in methods for treating diseases associated with PDE11A4 activity.

In one embodiment, the disclosure relates to a method of treating a disease alleviated by inhibiting PDE11A activity in a patient in need thereof, including administering to the patient a therapeutically effective amount of a PDE11A inhibitor. In one embodiment, the PDE11A inhibitor is a compound of formula (I), a compound of any one of formula (A), formula (I), formula (II), formula (III), a compound of any one of formula 1001-1729, formula 2001-2337, formula 3001-3031, or formula 4001-4729, or a pharmaceutically acceptable salt, solvate, hydrate, cocrystal, or prodrug thereof. In one embodiment, the disclosure relates to a method of treating a disease alleviated by inhibiting PDE11A4 activity in a patient in need thereof, including administering to the patient a therapeutically effective amount of a PDE11A4 inhibitor. In one embodiment, the PDE11A4 inhibitor is a compound of formula (I), a compound of any one of formula (A), formula (I), formula (II), formula (III), a compound of any one of formula 1001-1729, formula 2001-2337, formula 3001-3031, or formula 4001-4729, or a pharmaceutically acceptable salt, solvate, hydrate, cocrystal, or prodrug thereof. In some embodiments, the disease or disorder is associated with cognitive decline. In some embodiments, the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

In some embodiments, “cognitive decline” includes be any negative change in an animal's cognitive function. For example cognitive decline, includes but is not limited to, memory loss (e.g. behavioral memory loss), failure to acquire new memories, confusion, impaired judgment, personality changes, disorientation, or any combination thereof.

### Pharmaceutical Compositions

In an embodiment, an active pharmaceutical ingredient or combination of active pharmaceutical ingredients, such as any of the PDE11A inhibitors (e.g., PDE11A4 inhibitors) of the disclosure, is provided as a pharmaceutically acceptable composition. In some embodiments, the PDE11A inhibitor is a compound of formula (I), a compound of any one of formula (A), formula (I), formula (II), formula (III), a compound of any one of formula 1001-1729, formula 2001-2337, formula 3001-3031, or formula 4001-4729, or a pharmaceutically acceptable salt, solvate, hydrate, cocrystal, or prodrug thereof. In some embodiments, the PDE11A inhibitor is a PDE11A4 inhibitor. In some embodiments, the PDE11A4 inhibitor is a compound of formula (I), a compound of any one of formula (A), formula (I), formula (II), formula (III), a compound of any one of formula 1001-1729, formula 2001-2337, formula 3001-3031, or formula 4001-4729, or a pharmaceutically acceptable salt, solvate, hydrate, cocrystal, or prodrug thereof.

In one embodiment, the disclosure relates to a pharmaceutical composition including a therapeutically effective amount of a PDE11A inhibitor for the treatment of a disease alleviated by inhibiting PDE11A activity in a patient in need thereof, and a physiologically compatible carrier medium. In one embodiment, the disclosure relates to a pharmaceutical composition including a therapeutically effective amount of a PDE11A4 inhibitor for the treatment of a disease alleviated by inhibiting PDE11A4 activity in a patient in need thereof, and a physiologically compatible carrier medium. In some embodiments, the disease is associated with cognitive decline. In some embodiments, the PDE11A inhibitor is a compound of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof. In some embodiments, the PDE11A inhibitor is a PDE11A4 inhibitor. In some embodiments, the PDE11A4 inhibitor is a compound of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof. In some embodiments, the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

In some embodiments, the concentration of each of the active pharmaceutical ingredients provided in the pharmaceutical compositions of the disclosure, such as any of the compounds of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, %, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as a compound of formula (A), formula (I), formula (II), formula (III), a compound of any one of formula 1001-1729, formula 2001-2337, formula 3001-3031, formula 4001-4729, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the disclosure, such as any of the PDE11A inhibitors (e.g., PDE11A4 inhibitors) of the disclosure, for example a compound of formula (A), formula (I), formula (II), formula (III), a compound of any one of formula 1001-1729, formula 2001-2337, formula 3001-3031, formula 4001-4729, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v, or v/v of the pharmaceutical composition.

In some embodiments, the concentration of each of the active pharmaceutical ingredients provided in the pharmaceutical compositions of the disclosure, such as a compound of formula (A), formula (I), formula (II), formula (III), a compound of any one of formula 1001-1729, formula 2001-2337, formula 3001-3031, formula 4001-4729, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v, or v/v of the pharmaceutical composition.

In some embodiments, the amount of each of the active pharmaceutical ingredients provided in the pharmaceutical compositions of the disclosure, such as any of the foregoing compounds of formula (A), formula (I), formula (II), formula (III), a compound of any one of formula 1001-1729, formula 2001-2337, formula 3001-3031, formula 4001-4729, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the disclosure, such as any of the compounds of formula (A), formula (T), formula (TT), formula (ITT), a compound of any one of formula 1001-1729, formula 2001-2337, formula 3001-3031, formula 4001-4729, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the disclosure is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the PDE11A inhibitors (e.g., PDE11A4 inhibitors) of the disclosure may also be used if appropriate.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In an embodiment, the pharmaceutical compositions described herein, such as any of the PDE11A inhibitors (e.g., PDE11A4 inhibitors) of the disclosure, for example a compound of formula (A), formula (I), formula (II), formula (III), a compound of any one of formula 1001-1729, formula 2001-2337, formula 3001-3031, or formula 4001-4729, are for use in the treatment of a disease or disorder associated with cognitive decline. In an embodiment, the pharmaceutical compositions described herein, such as any of the PDE11A inhibitors (e.g., PDE11A4 inhibitors) of the disclosure, are for use in the treatment of dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), or cognitive decline associate with spatial memory, other depression-related behaviors, additional anxiety-related behaviors, sensorimotor gating, and social behaviors.

Described below are non-limiting pharmaceutical compositions and methods for preparing the same.

### Pharmaceutical Compositions for Oral Administration

In an embodiment, the disclosure provides a pharmaceutical composition for oral administration containing the active pharmaceutical ingredient or combination of active pharmaceutical ingredients, such as the PDE11A inhibitors (e.g., PDE11A4 inhibitors) described herein, and a pharmaceutical excipient suitable for oral administration.

In some embodiments, the disclosure provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient, and optionally (iv) an effective amount of a fourth active pharmaceutical ingredient.

In some embodiments, the pharmaceutical composition may be a liquid pharmaceutical composition suitable for oral consumption. Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl-lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolanmine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, a pharmaceutical composition is provided for injection containing an active pharmaceutical ingredient or combination of active pharmaceutical ingredients, such as a compound of formula (A), formula (I), formula (II), formula (III), a compound of any one of formula 1001-1729, formula 2001-2337, formula 3001-3031, or formula 4001-4729, and a pharmaceutical excipient suitable for injection.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, a pharmaceutical composition is provided for transdermal delivery containing an active pharmaceutical ingredient or combination of active pharmaceutical ingredients, such as compounds of formula (A), formula (I), formula (II), formula (III). a compound of any one of formula 1001-1729, formula 2001-2337, formula 3001-3031, formula 4001-4729, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445; and 5,001,139, the entirety of which are incorporated herein by reference. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra and a compound of formula (A), formula (I), formula (II), formula (III), a compound of any one of formula 1001-1729 formula 2001-2337, formula 3001-3031, formula 4001-4729. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions of a compound of formula (A), formula (I), formula (II), formula (III), a compound of any one of formula 1001-1729, formula 2001-2337, formula 3001-3031, formula 4001-4729, or a may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

### Kits

The disclosure also provides kits. The kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the disclosure provides a kit comprising a composition comprising a therapeutically effective amount of an active pharmaceutical ingredient (e.g., a compound of formula (A), formula (I), formula (II), formula (III), a compound of any one of formula 1001-1729, formula 2001-2337, formula 3001-3031, formula 4001-4729) or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the disclosure provides a kit comprising (1) a composition comprising a therapeutically effective amount of an active pharmaceutical ingredient (e.g., a compound of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof), or combination of active pharmaceutical ingredients, and (2) a diagnostic test for determining whether a patient's disease or disorder associated with cognitive decline is a particular subtype of a disease or disorder associated with cognitive decline. Any of the foregoing diagnostic methods may be utilized in the kit.

The kits described above are preferably for use in the treatment of the diseases and conditions described herein. In some embodiments, the kits are for use in the treatment of a disease or disorder associated with cognitive decline. In some embodiments, the kits are for use in the treatment of dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), or cognitive decline associate with spatial memory, other depression-related behaviors, additional anxiety-related behaviors, sensorimotor gating, and social behaviors.

### Dosages and Dosing Regimens

The amounts of the pharmaceutical compositions administered using the methods herein, such as the dosages of a compound of formula (A), formula (I), formula (II), formula (III), a compound of any one of formula 1001-1729, formula 2001-2337, formula 3001-3031, formula 4001-4729, or a pharmaceutically acceptable salt, solvate, hydrate. cocrystal, or prodrug thereof will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, the disclosure includes methods of treating a disease or disorder associated with cognitive decline in human subject suffering from the disease or disorder, the method comprising the steps of administering a therapeutically effective dose of an active pharmaceutical ingredient that is a compound of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof to the human subject.

In some embodiments, the disclosure includes methods of treating a disease or disorder associated with cognitive decline in a human subject suffering from the disease or disorder, the method comprising the steps of administering a therapeutically effective dose of an active pharmaceutical ingredient that is a compound of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof to the human subject to inhibit or decrease the activity of PDE11A protein.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients may continue as long as necessary. In selected embodiments, a pharmaceutical composition is administered for more than 1, 2, 3, 4, 5, 6, 7, 14, or 28 day(s). In some embodiments, a pharmaceutical composition is administered for less than 28, 14, 7, 6, 5, 4, 3, 2, or 1 day(s). In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein, for example any of the compounds of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is less than about 25 mg, less than about 50 mg, less than about 75 mg, less than about 100 mg, less than about 125 mg, less than about 150 mg, less than about 175 mg, less than about 200 mg, less than about 225 mg, or less than about 250 mg. In some embodiments, an effective dosage of an active pharmaceutical ingredient disclosed herein is greater than about 25 mg, greater than about 50 mg, greater than about 75 mg, greater than about 100 mg, greater than about 125 mg, greater than about 150 mg, greater than about 175 mg, greater than about 200 mg, greater than about 225 mg, or greater than about 250 mg.

In some embodiments, an effective dosage of an active pharmaceutical ingredient disclosed herein, for example any of the compounds of formula (A), formula (I), formula (II), formula (III), a compound of any one of formula 1001-1729, formula 2001-2337, formula 3001-3031, formula 4001-4729, or a pharmaceutically acceptable salt, solvate, hydrate, cocrystal, or prodrug thereof is in the range of about 0.01 mg/kg to about 200 mg/kg, or about 0.1 to 100 mg/kg, or about 1 to 50 mg/kg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect, e.g., by dividing such larger doses into several small doses for administration throughout the day. Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

The disclosure will be further described in the following embodiments, which do not limit the scope of the disclosure described in the claims.

Embodiment 1. A compound of formula (A), or a pharmaceutically acceptable salt, solvate, hydrate, cocrystal, or prodrug thereof:

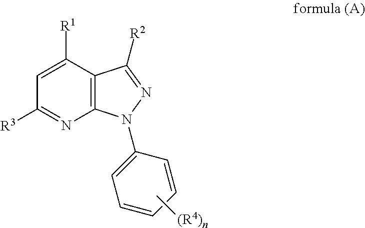

wherein in formula (A):
R¹ is selected from 5-membered heterocycle, 5-membered heteroaryl, 6-membered heterocycle, and 6-membered heteroaryl; or R¹ is

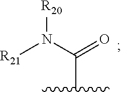

R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
R₂₀ is selected from H, optionally substituted alkyl, and optionally substituted haloalkyl;
R₂₁ is selected from optionally substituted alkyl and optionally substituted haloalkyl; or R₂₀ and R₂₁ are joined to form a 4-7 membered ring with the nitrogen to which they are each bound
n is an integer selected from 0-5; and
t is 1 or 2.

Embodiment 2. The compound of Embodiment 1, wherein the compound of formula (A) is a compound of formula (I), or a pharmaceutically acceptable salt, solvate, hydrate, cocrystal, or prodrug thereof:

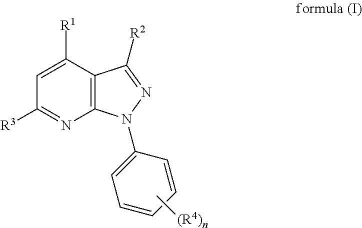

wherein in formula (I):
R¹ and R³ are independently at each occurrence a 5-membered heterocycle, 5-membered heteroaryl, a 6-membered heterocycle, or 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

Embodiment 3. The compound of Embodiment 1 or Embodiment 2, wherein is R¹ is a 5-membered heteroaryl ring, optionally selected from thiazole, oxazole, imidazole, thiadiazole, oxadiazole, and triazole.

Embodiment 4. The compound of any one of Embodiments 1 to 3, or pharmaceutically acceptable salt, solvate, hydrate, cocrystal, or prodrug thereof, wherein the compound is a compound of formula (II):

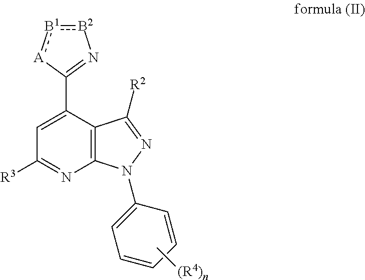

wherein in formula (II):
A is S, NR⁵, N, or O, optionally A is S or O;
B¹ and B² are independently at each occurrence selected from N, O, or CR⁵, wherein B¹ is not O when either A or B² is O; and
each R⁵ is independently at each occurrence selected from hydrogen, C₁-C₆ alkyl (e.g., C₁-C₃ alkyl), and C₁-C₃ haloalkyl (e.g., CH₂F, CHF₂, CF₃);
R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
n is an integer selected from 0-5; and
t is 1 or 2.

Embodiment 5. The compound of Embodiment 4, wherein R¹ is selected from

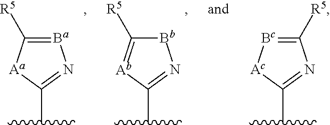

wherein:
A^{a} is O or S, and B^{a} is N or CH;
A^{b} is N, and B^{b} is O or S;
A^{c} is O or S, and B° is N; and
R⁵ is selected from hydrogen, C₁-C₃ alkyl (e.g., methyl, ethyl, propyl, isopropyl), and C₁-C₃ haloalkyl (e.g., CH₂F, CHF₂, or CF₃).

Embodiment 6. The compound of Embodiment 4 or Embodiment 5, wherein R¹ is selected from

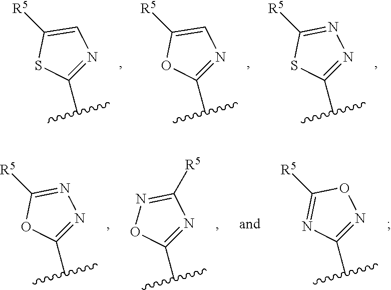

and R⁵ is selected from H, methyl, ethyl, propyl, isopropyl, CH₂F, and CHF₂.

Embodiment 7. The compound of any one of Embodiments 4-6, wherein R¹ is selected from

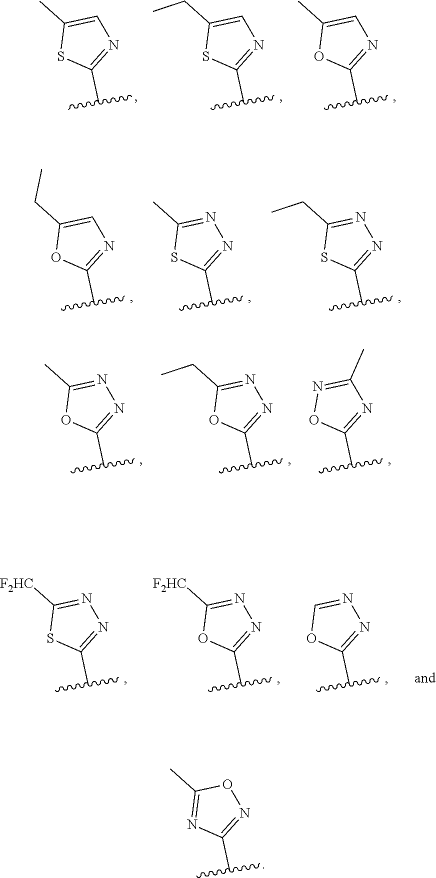

Embodiment 8. The compound of any one of Embodiments 1-7, wherein R₂ is selected from hydrogen, C₃-C₆ cycloalkyl, C₁-C₆ alkyl (e.g., C₁-C₄ straight chain alkyl, C₃-C₆ branched alkyl), —CF₃, —CH₂F, —CHF₂, and —C(R⁶R⁷)ₚX;
wherein, p is an integer from 1-3;
R⁶ and R⁷ are each independently selected from hydrogen, C₁-C₃ alkyl, and C₃-C₆ cycloalkyl;
X is selected from —OH, —CN, —OR⁸, and NR⁹R¹⁰;
R⁸ is selected from C₁-C₃ alkyl (e.g., C₁-C₃ straight or branched alkyl), and C₃-C₆ cycloalkyl; and
R⁹ and R¹⁰ are each independently selected from hydrogen, C₁-C₃ alkyl, and C₃-C₆ cycloalkyl.

Embodiment 9. The compound of Embodiment 8, wherein R₂ is selected from hydrogen, C₁-C₄ alkyl (e.g., methyl, ethyl, propyl, isopropyl, butyl, tertbutyl), —CF₃, —CH₂F, and —CHF₂.

Embodiment 10. The compound of Embodiment 8 or Embodiment 9, wherein R₂ is selected from H, —CH₃, —CF₃, and —CHF₂.

Embodiment 11. The compound of any one of Embodiments 1-10, wherein is R³ is a 5-membered heteroaryl ring, optionally selected from pyrrole, thiophene, furan, pyrazole, thiazole, isothiazole, oxazole, isoxazole, and imidazole.

Embodiment 12. The compound of Embodiment 11, wherein R³ is selected from

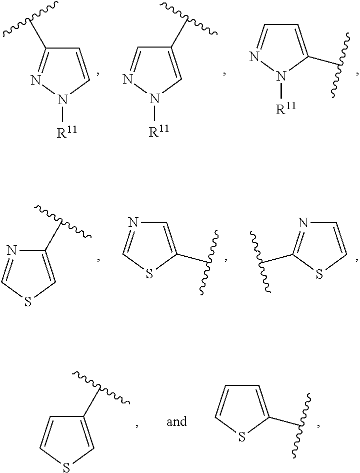

wherein R¹¹ is selected from hydrogen, C₁-C₃ alkyl (e.g., methyl, ethyl, propyl, isopropyl), C₃-C₆ cycloalkyl, CF₃, CH₂CF₃, CH₂CHF₂, and CH₂CH₂F.

Embodiment 13. The compound of Embodiment 12, wherein R³ is selected from

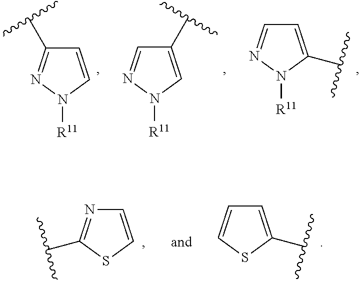

Embodiment 14. The compound of Embodiment 12 or Embodiment 13, wherein R¹¹ is selected from hydrogen, methyl, ethyl, propyl, isopropyl, —CF₃, and —CH₂CF₃.

Embodiment 15. The compound of any one of Embodiments 11-14, wherein R³ is selected from

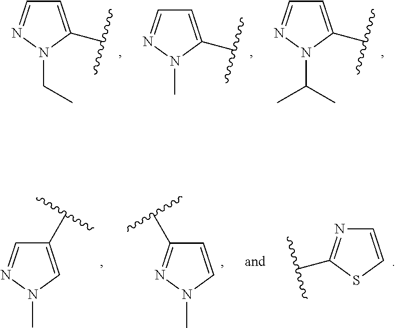

Embodiment 16. The compound of any one of Embodiments 11-15, wherein R³ is selected from

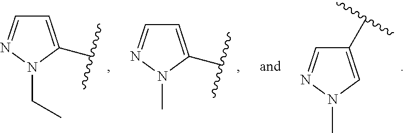

Embodiment 17. The compound of any one of Embodiments 1-16, wherein R⁴ is selected from hydrogen, C₁-C₆ alkyl, and halo (e.g., F or Cl).

Embodiment 18. The compound of any one of Embodiments 1-17, wherein n is 0, 1, or 2.

Embodiment 19. The compound of Embodiment 18, wherein n is 0 or 1.

Embodiment 20. The compound of any one of Embodiments 1-19, wherein R⁴ is selected from hydrogen, 4-fluoro, 3-fluoro, 2-fluoro, 2-chloro, and 4-chloro.

Embodiment 21. The compound of any one of Embodiments 1-20, wherein R⁴ is selected from hydrogen, 4-fluoro, 2-fluoro, and 2-chloro.

Embodiment 22. The compound of any one of Embodiments 1-18, wherein n is 2 and each R⁴ is 2-fluoro and 4-fluoro.

Embodiment 23. The compound of any one of Embodiments 1-4, wherein the compound of formula (A), formula (I), or formula (II) is a compound having any one of formula 1001-1080, 1097-1176, 1193-1272, 1289-1368, or 1385-1729, or a pharmaceutically acceptable salt, solvate, hydrate, cocrystal, or prodrug thereof.

Embodiment 24. The compound of any one of Embodiments 1-4, wherein the compound of formula (A), formula (I), or formula (II) is a compound having any one of formula 2001-2337, or a pharmaceutically acceptable salt, solvate, hydrate, cocrystal, or prodrug thereof.

Embodiment 25. The compound of any one of Embodiments 1-4, wherein the compound of formula (A), formula (I), or formula (II) is a compound having any one of the formula of Compounds 1013, 1014, 1109, 1110, 1015, 1625, 1111, 1011, 1009, 1107, 1105, 2217, 2218, 2008, 2210, 2315, 2219, 1682, 1691, 2017, 2015, 2016, 2087, 2019, 2001, 2020, 2021, 2222, 2215, 1448, 1543, 2264, 1393, 1717, 2278, 2027, 2041, 1103, 1101, or a pharmaceutically acceptable salt, solvate, hydrate, cocrystal, or prodrug thereof.

Embodiment 26. The compound of Embodiment 1, or pharmaceutically acceptable salt, solvate, hydrate, cocrystal, or prodrug thereof, wherein:
R¹ is

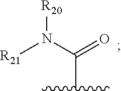

and
R₂₀ is selected from H, optionally substituted C₁-C₆ alkyl, and optionally substituted C₁-C₆ haloalkyl; R₂₁ is selected from optionally substituted C₁-C₆ alkyl and optionally substituted C₁-C₆ haloalkyl;
or R₂₀ and R₂₁ are joined to form a 4-6-membered heterocycle with the nitrogen to which they are each bound.

Embodiment 27. The compound of Embodiment 1 or 26, or pharmaceutically acceptable salt, solvate, hydrate, cocrystal, or prodrug thereof, wherein R₂₀ is selected from H, methyl, ethyl, propyl, isopropyl, butyl, tert-butyl, CF₃, CHF₂, CH₂F, CH₂CF₃, CH₂CHF₂, and CH₂CH₂F; and R₂₁ is selected from methyl, ethyl, propyl, isopropyl, butyl, tert-butyl, CF₃, CHF₂, CH₂F, CH₂CF₃, CH₂CHF₂, and CH₂CH₂F;
or R₂₀ and R₂₁ are joined to form a 4-6-membered heterocycle with the nitrogen to which they are each bound.

Embodiment 28. The compound of Embodiment 1, 26, or Embodiment 27, or a pharmaceutically acceptable salt, solvate, hydrate, cocrystal, or prodrug thereof, wherein R₂₀ is selected from H, methyl, ethyl, propyl, tert-butyl, and CH₂CH₂F; and R₂₁ is selected from methyl, ethyl, propyl, tert-butyl, and CH₂CH₂F;
or R₂₀ and R₂₁ are joined to form a 4-6-membered heterocycle with the nitrogen to which they are each bound.

Embodiment 29. The compound of Embodiment 1 or Embodiment 26, or pharmaceutically acceptable salt, solvate, hydrate, cocrystal, or prodrug thereof, wherein the compound is of formula (III):

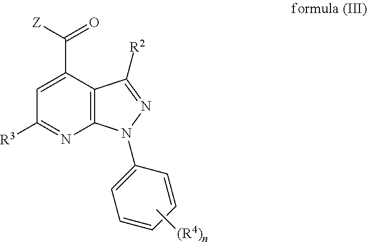

wherein in formula (III):
Z is selected from

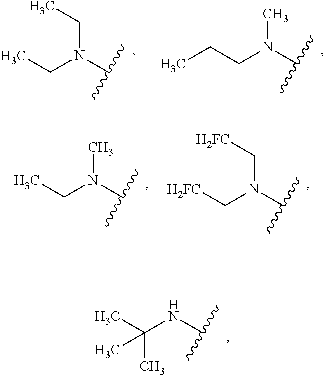

and
R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

Embodiment 30. The compound of any one of Embodiments 1 or 26-29, wherein R₂ is selected from hydrogen, C₃-C₆ cycloalkyl, C₁-C₆ alkyl (e.g., C₁-C₄ straight chain alkyl, C₃-C₆ branched alkyl), —CF₃, —CH₂F, —CHF₂, and —C(R⁶R⁷)ₚX;
wherein, p is an integer from 1-3;
R⁶ and R⁷ are each independently selected from hydrogen, C₁-C₃ alkyl, and C₃-C₆ cycloalkyl;
X is selected from —OH, —CN, —OR⁸, and NR⁹R¹⁰;
R⁸ is selected from C₁-C₃ alkyl (e.g., C₁-C₃ straight or branched alkyl), and C₃-C₆ cycloalkyl; and
R⁹ and R¹⁰ are each independently selected from hydrogen, C₁-C₃ alkyl, and C₃-C₆ cycloalkyl.

Embodiment 31. The compound of Embodiment 30, wherein R₂ is selected from hydrogen, C₁-C₃ alkyl (e.g., methyl, ethyl, propyl, isopropyl), —CF₃, —CH₂F, and —CHF₂.

Embodiment 32. The compound of Embodiment 30 or Embodiment 31, wherein R₂ is selected from hydrogen and —CH₃.

Embodiment 33. The compound of any one of Embodiments 1 or 26-32, wherein is R³ is a 5-membered heteroaryl ring, optionally selected from pyrrole, thiophene, furan, pyrazole, thiazole, isothiazole, oxazole, isoxazole, and imidazole.

Embodiment 34. The compound of Embodiment 33, wherein R³ is selected from

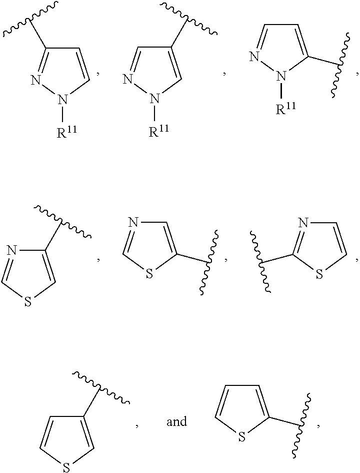

wherein R¹¹ is selected from hydrogen, C₁-C₃ alkyl (e.g., methyl, ethyl, propyl, isopropyl), C₃-C₆ cycloalkyl, CF₃, CH₂CF₃, CH₂CHF₂, and CH₂CH₂F.

Embodiment 35. The compound of Embodiment 34, wherein R³ is selected from

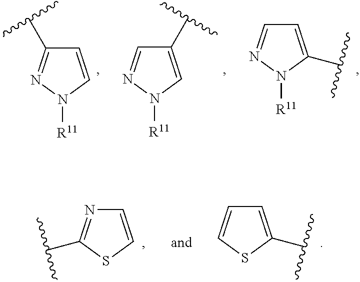

Embodiment 36. The compound of Embodiment 34 or Embodiment 35, wherein R¹¹ is selected from hydrogen, methyl, ethyl, propyl, isopropyl, —CF₃, and —CH₂CF₃.

Embodiment 37. The compound of any one of Embodiments 1 or 26-36, wherein R³ is selected from

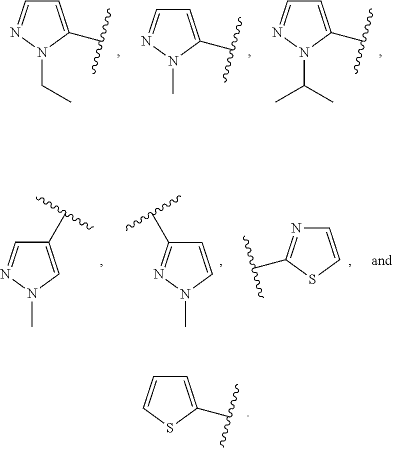

Embodiment 38. The compound of any one of Embodiments 1 or 26-37, wherein R⁴ is selected from hydrogen, C₁-C₆ alkyl, and halo (e.g., F or Cl).

Embodiment 39. The compound of any one of Embodiments 1 or 26-38, wherein n is0 or 1.

Embodiment 40. The compound of any one of Embodiments 1 or 26-39, wherein R⁴ is selected from hydrogen, 4-fluoro, 3-fluoro, 2-fluoro, 2-chloro, and 4-chloro.

Embodiment 41. The compound of any one of Embodiments 1 or 26-40, wherein R⁴ is selected from hydrogen, 4-fluoro, 3-fluoro, 2-fluoro, and 4-chloro.

Embodiment 42. The compound of Embodiment 1 or 29, wherein the compound of formula (III) is a compound having any one of formula 3001, 3006-3021, or 3023-3031, or a pharmaceutically acceptable salt, solvate, hydrate, cocrystal, or prodrug thereof:

Embodiment 43. The compound of any one of Embodiments 1 to 42, wherein the compound is a PDE11A4 inhibitor.

Embodiment 44. The compound of Embodiment 43, wherein the PDE11A4 inhibitor is a PDE11A4 selective inhibitor.

Embodiment 45. A pharmaceutical composition comprising a compound of any one of Embodiments 1 to 43, or a pharmaceutically acceptable salt, solvate, hydrate, cocrystal, or prodrug thereof, and a physiologically compatible carrier medium.

Embodiment 46. A pharmaceutical composition comprising a compound of any one of Embodiments 1 to 43, or a pharmaceutically acceptable salt, solvate, hydrate, cocrystal, or prodrug thereof, and a physiologically compatible carrier medium, wherein the amount of the compound in the composition is a therapeutically effective amount for the treatment or prevention of a disease or disorder alleviated by inhibiting PDE11A4 activity in a patient in need thereof.

Embodiment 47. The pharmaceutical composition of Embodiment 45 or Embodiment 46, wherein the disease or disorder is associated with cognitive decline.

Embodiment 48. The pharmaceutical composition of Embodiment 47, wherein the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

Embodiment 49. A method of treating or preventing a disease or disorder alleviated by inhibiting PDE11A4 activity in a patient in need thereof, comprising administering to the patient a therapeutically effective amount of a compound of any one of Embodiments 1 to 43.

Embodiment 50. A method of treating or preventing a disease or disorder alleviated by inhibiting PDE11A4 activity in a patient in need thereof, comprising administering to the patient a therapeutically effective amount of a pharmaceutical composition of any one of Embodiments 45-48.

Embodiment 51. The method of any one of Embodiments 49 or 50, wherein the isolated fragment is administered in a dosage unit form.

Embodiment 52. The method of Embodiment 51, wherein the dosage unit comprises a physiologically compatible carrier medium.

Embodiment 53. The method of any one of Embodiments 49-52, wherein the disease or disorder is associated with cognitive decline.

Embodiment 54. The method of Embodiment 53, wherein the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.

The following examples describe the disclosure in further detail. These examples are provided for illustrative purposes only, and should in no way be considered as limiting the disclosure.

### EXAMPLES

### Example 1: Phosphodiesterase 11A (PDE11A) and its Role in the Neurobiological Substrates of Memory and Social Behaviors

This Example describes data testing the hypothesis that age-related increases in HIPP PDE11A4 occur in a compartmentalized manner and impair social aLTMs. A novel conditional transgenic system that controls the expression of PDE11A4 in a time- and brain region-specific manner by combining overexpression lentiviruses with PDE11A WT and KO mice is used. RNAscope probes are used to delineate nuclear vs. cytosolic localization of PDE11A4 mRNA. In vivo/ex vivo techniques are used to study of PDE compartmentalization—and its functional consequences—rather than the study of artificial FRET-based constructs in in vitro assays.

Given PDE11A4's uniquely restricted expression pattern, the studies not only uncover the neurobiological role of a particular enzyme, they also define the function of an anatomically restricted, molecularly defined-circuit that appears to be uniquely specialized for processing social information. The focus on LTMs for social experiences is relatively unique in the field of learning memory. Most studies tend to utilize contextual/cued fear conditioning, novel object recognition or various spatial learning paradigms (e.g., water maze). Further, what studies have examined the molecular/anatomical mechanisms of social memory have largely focused on short-term memory (STM). The role that age-related increases in PDE11A4 plays in STM, recent LTM (24 hthes after training), and remote LTM (7 days after training) of both aLTMs and rLTMs for social vs. non-social experiences is compared. An Integrative approach includes the use of in vivo, ex vivo, and in vitro approaches, along with viral techniques, permitting assessing translatability of findings across species.

Determining if Preventing or Reversing Age-Related Increases in PDE11A4 can Prevent and/or Rescue Age-Related Impairments in Social aLTM.

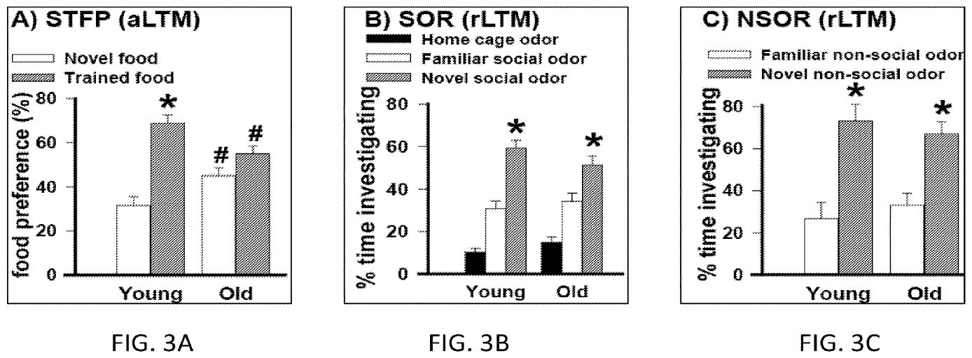

Age-related cognitive decline is not a uniform process, with variability in symptom severity observed across cognitive domains. Human studies have demonstrated that associative long-term memories (aLTMs)—particularly those involving experiences with family and friends—are more susceptible to age-related cognitive decline than are recognition long-term memories (rLTMs). This differential sensitivity of social aLTMs vs rLTMs in mice (FIG. 3) was recapitulated. The findings, in combination with the human studies noted above and a report showing rapid decay of social aLTMs in aged rats, suggests that an enhanced vulnerability of social aLTMs to age-related cognitive decline is conserved across species.

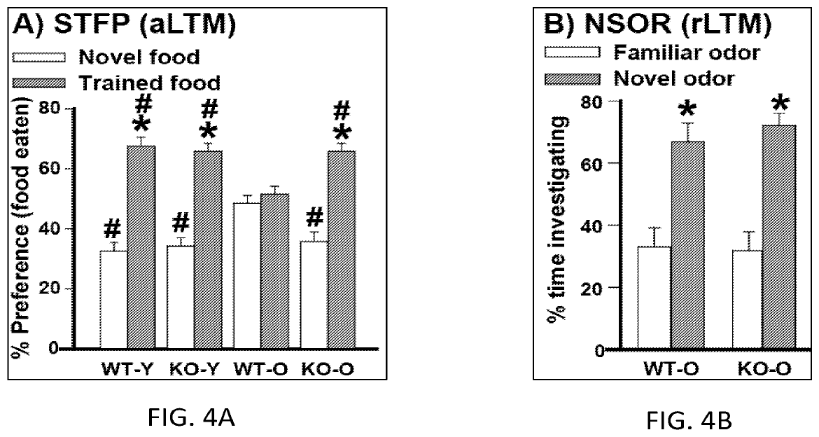

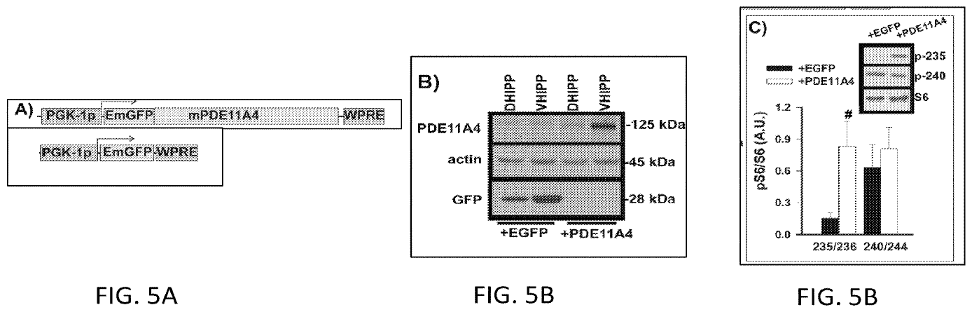

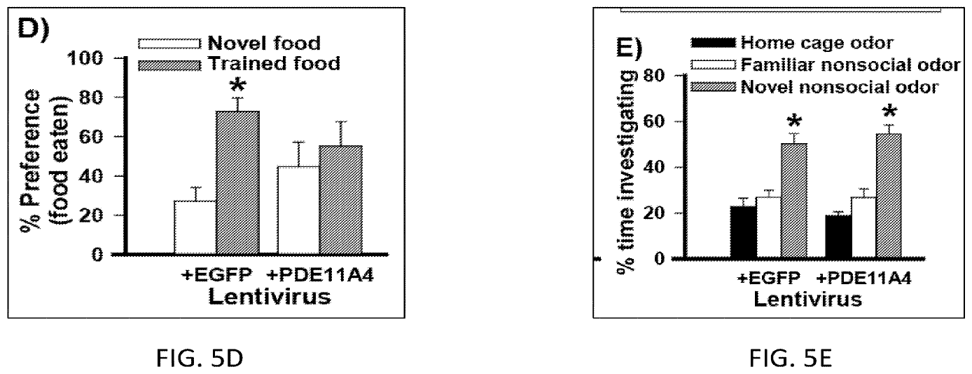

To test the hypothesis that age-related cognitive decline of social aLTMs is driven by the age-related increases in PDE11A4 expression described above (FIGS. 2A-2C), young vs. old PDE11A WT and KO mice in social transmission of food preference (STFP; FIGS. 4A-4B) were compared. While aging severely impairs PDE11A WT mice, old KO mice show robust aLTM for STFP on par with that of young PDE11A WT mice. These findings have been replicated in two large cohorts of male and female mice (no effect of sex; combined data shown in FIGS. 4A-4B), underscoring the reproducibility of the protective effect. Further, in preliminary studies, it has been shown that the protective effect of PDE11A deletion is reversible by acutely overexpressing PDE11A4 in the hippocampus of PDE11A KO mice (i.e., mimicking the state of an old WT; FIGS. 5A-5E). Together, these data show that preventing age-related increases in PDE11A4 is sufficient to prevent age-related cognitive decline of social aLTMs and suggests that more acute manipulations of elevated PDE11A4 expression is meet with equal success.

Based on data disclosed herein, preventing or minimizing age-related increases in PDE11A4 is sufficient to prevent the onset of age-related cognitive decline of social aLTMs (FIGS. 4A-4B). Further, the age-related cognitive decline observed in PDE11A WTs is due to the excessive PDE11A4 that is acutely present in the aged adult hippocampus. Identifying when and where PDE11A4 affects age-related cognitive decline allowed for the discovery of more sophisticated therapeutic approaches to target PDE11A4 for the reversal of aLTM deficits in aged adults or only as a prophylactic agent that prevents the onset of deficits.

### Experimental Approach:

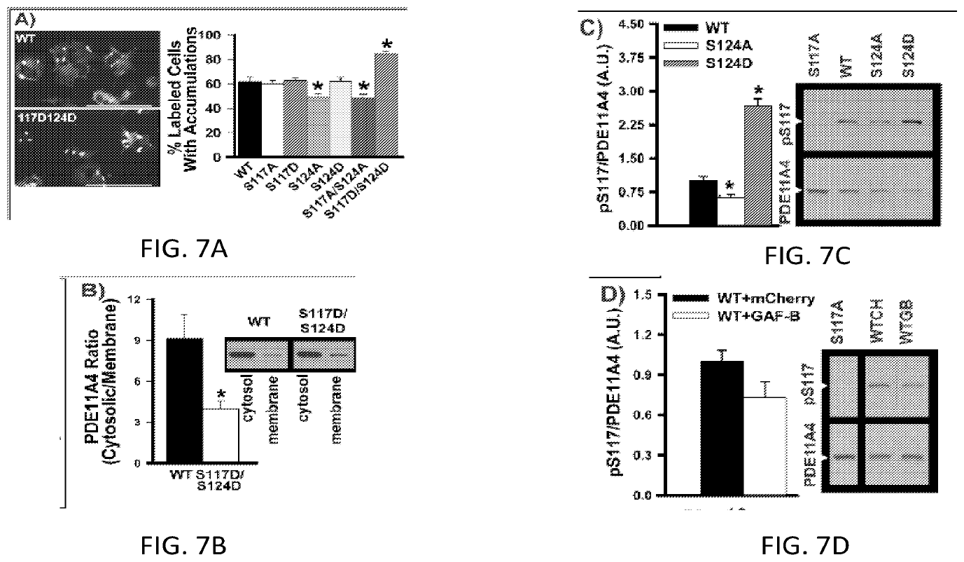

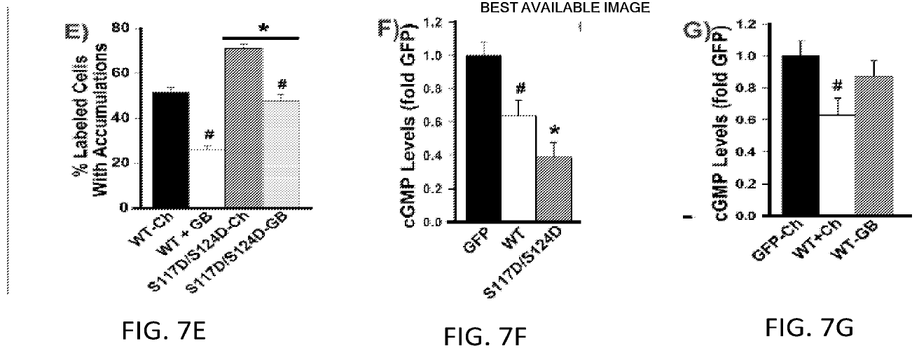

Innovative conditional transgenic system used to prevent, reverse, or mimic age-related increases in HIPP PDE11A4: This system combines PDE11A WT and KO mice with lentivirus constructs that either overexpress (FIGS. 5A-5E). Deletion of PDE11A does not appear to trigger compensatory upregulation of closely related PDEs. In a non-limiting embodiment, integrative studies are conducted with a number of mutant mouse models and lentiviruses. In a non-limiting example, “young” is defined as 2-6 months of age and “Old” is defined as 18-22 months of age since 1) protective effects are observed in PDE11A KO mice as early as 14 months old and 2) it is expected 50% of the colony dies by the age of 24 months (i.e., do not want to introduce a selection bias by preferentially studying older mice with exceptional longevity). In the mimic experiments, PDE11A WT or KO mice are administered a lentivirus containing a control fluorescent protein (EmGFP, as appropriate) or an N-terminal EmGFP-tagged PDE11A4 construct. It was chosen to place the tag at the N-terminus in view of the benign nature of a PDE11A4 N-terminal tag, which have been confirmed (FIGS. 7F-7G). Untreated male and female PDE11A WT, HT and KO mice are used to study the effects of preventing (in the case of the KO) or minimizing (in the case of the HT) age-related increases in PDE11A4 expression (FIGS. 4A-4B).

Viral vectors were delivered using stereotaxic techniques similar to those that have been described previously, except that injections were made directly into the hippocampus. Coordinates were selected to target CAT and subiculum—the portions of the hippocampus that naturally express PDE11A4 (DHIPP coordinates: AP −1.7 mm, ML+/−1.6 mm, DV −1.4 mm; VHIPP coordinates: AP−3.3 mm, ML+/−3.5 mm, DV −4.4 mm). The ability to virally manipulate PDE11A4 expression has been measured and function in vivo and to measure aLTM and rLTM in virally-treated mice (FIGS. 5A-5E). These data support the hypothesis that reversal/mimicry of age-related increases in PDE11A4 expression in the adult hippocampus is sufficient to rescue/cause deficits in social aLTMs.

Social aLTM using STFP were measured and—to determine the specificity of the effects-1) non-social aLTM using contextual fear conditioning, 2) social rLTM using SOR, and 3) non-social rLTM using NSOR. To date, experiments measuring remote aLTM for STFP (i.e., 7 days post training; FIGS. 4A-4B and 5A-5E) have been prioritized as opposed to short-term memory (STM; 1 the post training) or recent long-term memory (24 hours post training) for 2 reasons. First, a study in rats suggested that aging impairs aLTM for STFP at more remote time points but not immediately after training. Second, adolescent and young adult PDE11A KO mice show intact STM and remote LTM (FIGS. 4A-4B and 5A-5E), but impaired recent LTM for social experiences. The studies to date indicate that PDE11A KO mice exhibit transient amnesia by virtue of expediting systems consolidation, which temporarily “misplaces” the memory but ultimately results in a strengthened memory trace in the cortex. While it remains to be determined if old PDE11A KO mice similarly show a form of transient amnesia (intact STM, impaired recent LTM, improved remote LTM), such an effect on systems consolidation could explain why old KOs are protected from age-related cognitive decline (see below for Arc mapping study that tests for this possibility). That said, Young PDE11A heterozygous (HT) mice do show recent LTMs for social experiences, and yet old PDE11A HTs are still protected against age-related cognitive decline of aLTMs for STFP (HT-O: novel food, 35.9 z3.9%; trained food 64.1 z3.9%; P<0.001). Thus, transient amnesia is not required to see the protective effects of PDE11A deletion on remote aLTMs in aged mice.

Moving forward, young vs. old WT, HT, and KO PDE11A mice are compared at all 3 time points (STM, recent LTM, and remote LTM) to determine the effects of age-related increases in PDE11A4 at each memory phase. In addition to behavior, ex vivo studies are conducted. Functional activation of neural circuits was mapped using in situ hybridization for the activity-regulated gene Arc, as has been previously disclosed. In so doing, the circuit was identified (including nodes of activity and functional connectivity amongst nodes) that is engaged by aged PDE11A KO mice during the successful retrieval of a social aLTM. It is then determined if the circuit engaged by an aged PDE11A KO shares similarities with or diverges from that of young PDEt tA WT mice. This approach shows whether preventing age-related increases in PDE11A prevents/delays the onset of aging pathophysiology or if it simply enables the brain to adopt a compensatory strategy for achieving equivalent behavioral performance (i.e., engages cognitive reserve). It is also determined if preventing/reversing age-related increases in PDE11A4 is sufficient to prevent/reverse age-related changes in phosphorylation of CREB, GluR1, and CaMKII that it has been reported to occur in the hippocampus. Cause-and-effect studies determine which of these downstream events may be sufficient to drive age-related cognitive decline of social aLTMs.

In conducting these ex vivo studies, cannula tracks and PDE11A4 expression levels are verified in each virally-treated subject. If a viral delivery is determined to have failed (e.g., cannula tracks miss the hippocampus, PDE11A4 expression not changed) data from that subject are dropped. Approximately equal numbers of male and female offspring are used in all studies (see infra for specific n's). All experiments are counterbalanced for sex and genotype, but data are collected by an experimenter blind to genotype.

### Results

Based on the fact that PDE11A HT and KO mice are protected against age-related decline of aLTMs (FIGS. 4A-4B), it is anticipated that age-related increases in PDE11A4 expression are deleterious. Although not wishing to be bound by any particular thory, the fact that the protective effect of the KO is acutely reversed, suggests that the deleterious effects of PDE11A4 are due to the acute presence of elevated PDE11A4 in the aged brain (as opposed to a cumulative effect of chronic overexpression). This suggests that knocking down elevated PDE11A4 expression in the aged hippocampus are as effective as preventing age-related increases in PDE11A4 expression. This interpretation are based on two primary results. First, virally overexpressing PDE11A4 expression in old PDE11A KO or young WT mice (i.e., mimicking the state of an old WT) are sufficient to cause deficits in social aLTMs. Second, virally knocking down PDE11A4 expression in aged WTs are sufficient to rescue social aLTM deficits. Such a profile is interpreted as proof in favor of pursuing PDE11A4 inhibitors as potential therapeutics for age-related decline of social aLTMs. It are particularly interesting to determine if aged PDE11A KO mice exhibit transient amnesia for social memories as it has been observed in young KOs. If old PDE11A KO mice show intact or improved recent LTM, this suggests that the function of PDE11A4 evolves across the lifespan. There is precedence for proteins related to PDE signaling showing this type of “developmental switch” across the lifespan. See infra for statistical methods.

Seventeen month-old C₅₇BL/6J mice have been successfully surgerized, so experiencing general difficulties with conducting stereotaxic surgeries in old mice is not anticipated. In a non-limiting example, it is possible the aged brain may mount an immune response to the lentiviral constructs that is not typically seen in young adult mice. In a non-limiting example, in the case of the former (as per elevated IBA-1, GFAP, or IL-6 staining), the construct is moved to an adeno-associated virus. In another non-limiting example, a conditional transgenic is developed and backcrossed onto the PDE11A line to allow reversible overexpression of PDE11A4 in WTs and KOs using doxycycline. In another non-limiting example, a conditional knockout approach is pursued using an inducible FLEX-Cre recombinase system.

Identify the Circuit, Cell-Type and Subcellular Domain where PDE11A4 is Upregulated with Age and the Signal Driving this Compartmentalized Effect.

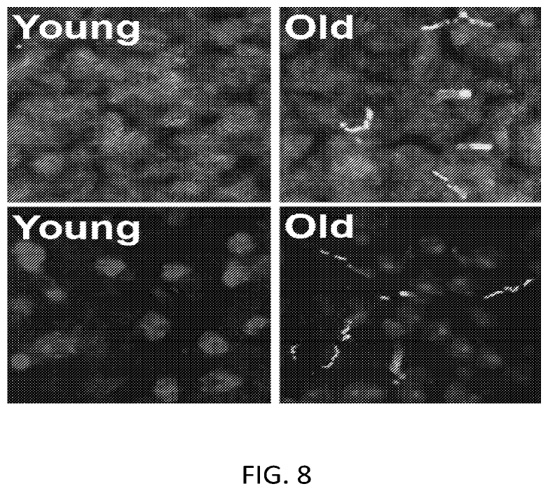

Intramolecular signals have been identified that alter the trafficking of PDE11A4, including homodimerization and N-terminal phosphorylation (FIGS. 7A-7G). As such, it is important to determine 1) if the rate/nature of PDE11A4 post-translational modifications change with age and, as a result, 2) if age-related increases in PDE11A4 expression occur in a compartment-specific manner (i.e., if excessive PDE11A4 is aberrantly trafficked). In a preliminary study, PDE11A4 were mapped expression in young vs old C₅₇BL/6J and BALB/cJ mice by immunofluorescence and found in both strains and sexes that the excessive expression of PDE11A4 protein that occurs with age appears to accumulate in short “filamentous structures” (FIG. 8). These PDE11A4-filled structures are rarely seen in young mice but are abundant in CAT, AHi, and especially the subiculum of aged mice. The preliminary study also shows that aging dramatically increases phosphorylation of PDE11A4 at serines 117 and 124 (pS117/pS124) and that pS117/pS124 is found almost exclusively in the pools of PDE11A4 that accumulate in these filamentous structures (FIG. 8). This in vivo finding is consistent with the in vitro studies showing that the phosphomimic mutation S117D/S124D increases accumulation of PDE11A4 while the phosphoresistant mutation ST17A/S124A has the opposite effect (FIG. 7A).

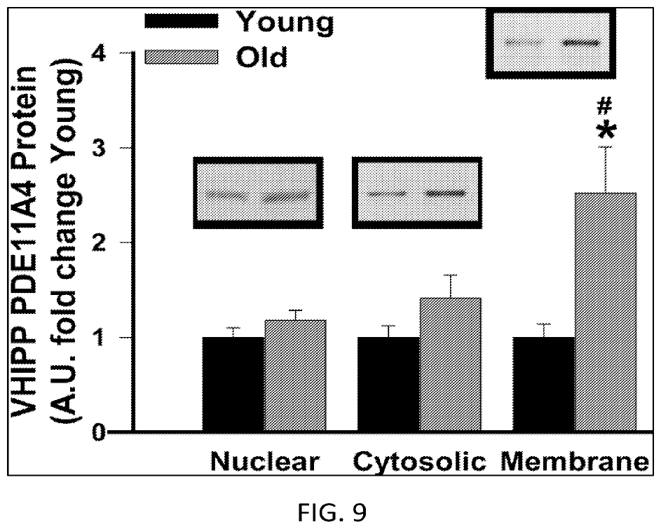

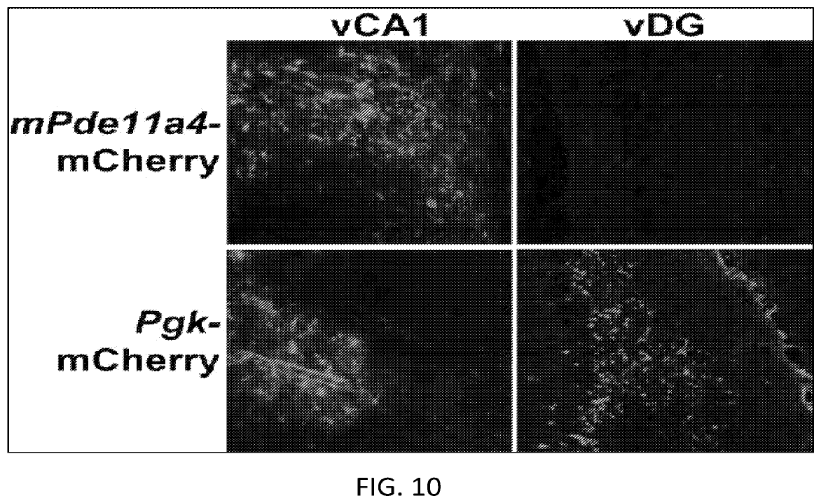

A preliminary biochemical fractionation experiment also points to compartment-specific effects. Age-related increases in VHIPP PDE11A4 occur primarily in the membrane fraction as opposed to the cytosol or nucleus, suggesting a mislocalization of the overexpressed PDE11A4 (FIG. 9). Although not wishing to be bound by any particular theory, this mislocalization is likely driven by the increase in PDE11A4-pS117/pS124 because the in vitro studies show that S117D/S124D shifts PDE11A4 from the cytosol to the membrane (FIG. 7B). Not only does S117D/S124D change the localization of PDE11A4, it also selectively increases PDE11A4 cGMP hydrolytic activity, thus causing deficits in cGMP that mimic aging (FIG. 7F). Together, these data suggest that age-related increases in PDE11A4 expression are compounded by increased cGMP hydrolytic activity and a mislocalization of that aberrant activity that is driven by phosphorylation at S117/S124.

Unlike S117D/S12D, disrupting PDE11A4 homodimerization reduces PDE11A4 accumulation and shifts PDE11A4 from the membrane back to the cytosol. Importantly, disrupting PDE11A4 homodimerization also reduces PDE11A4 cGMP hydrolytic (FIG. 7G), consistent with the fact that it reduces pS117/pS124-PDE11A4 (FIG. 7D). As such, studies here determines if preventing phosphorylation at S117/S124 or disrupting homodimerization are sufficient to prevent/reverse 1) age-related accumulation of PDE11A4 in filamentous structures and/or the membrane and 2) age-related deficits in social aLTMs.

Previously it was shown that PDE11A4 protein expression increases in hippocampal lysates, but the specific compartment within which this increase occurs has yet to be identified. It is possible that this excess PDE11A4 may not be globally distributed, but rather discretely localized or even ectopically expressed in cell types or subcellular domains that are normally void of PDE11A4. Defining the signals that control age-related changes in PDE11A4 trafficking enables a more sophisticated targeting of compartment-specific localizations of the enzyme as opposed to overall catalytic activity, which is preferred since eliminating all PDE11A catalytic activity influences social preferences and impairs recent LTMs for social experiences.

These results demonstrated that age-related increases in pS117/pS124 cause PDE11A4 to accumulate ectopically, contributing to age-related decline of social aLTMs.

### Experimental Approach:

Series 1: Identifying the location of age-related increases in PDE11A4 at the level of circuit, cell type, and subcellular domain and the intramolecular signals driving those discrete changes. To establish the conservation of effects across species, these experiments utilize brains from young vs old 1) C₅₇BL/6 and BALB/cBy mice, 2) Fischer 344 and Brown Norway rats, and 3) rhesus monkeys (all obtained from the NIA Rodent Colonies or Primate Tissue Bank). Mice and rats are available as live animals and, thus, are tested for remote LTM of STFP, SOR, and NSOR to confirm a selective age-related cognitive decline of social aLTMs. Monkey tissue is available only from post mortem stock and, thus, subjects are not cognitively phenotyped. As noted above, age-related expression changes in PDE11A4 do appear to be conserved between rodents and primates at the level of total HIPP mRNA levels (rat:human: FIG. 2B); this is verified at the level of PDE11A4 protein expression and compartmentalization. Tissue from PDE11A KO mice are processed in parallel as a negative control.

Defining changes at the level of circuit and cell type. As per the published techniques, the first hemisphere (rodents) or block of tissue (primate) are kept intact for processing by in situ hybridization and IF in order to determine in which hippocampal subfields age-related increases in PDE11A4 mRNA, protein expression, and phosphorylation occur (e.g., CAT vs. subiculum or stratum radiatum vs. stratum pyramidale). As noted above, changes in the subcellular location of a PDE would impact function—but so would changes at the level of circuit or cell type. For example, changes in subiculum would indicate an effect on retrieval mechanisms; whereas, changes in CAT would indicate an effect on input integration. More specifically, changes in CA1 dendrites of stratum radiatum proximal to the cell body indicates modulation of CA3 input signals, while changes in distal CA1 dendrites indicates modulation of entorhinal cortex input signals. As described above, the age-related increases in PDE11A4 expression most strikingly occur in the filamentous structures; however, the preliminary study also showed that a subset of sporadically distributed neurons in the VHIPP stratum pyramidale exhibit increased PDE11A4 expression around the cell body. The sporadic nature of these cell bodies raises the possibility that these neurons reflect either a specific subtype of inhibitory interneuron or neurons that send projections to a discrete brain region. In young mice, it was found only PDE11A4 expressed in excitatory neurons of CA1, subiculum, and the AHi. The preliminary IF study described above verifies that PDE11A4 expression continues to be restricted to these subfields in old mice but it has not been verified that PDE11A4 is only expressed in excitatory neurons in old mice. If PDE11A4 were to become ectopically expressed in inhibitory interneurons with age, this has significant functional consequences for the excitatory-inhibitory balance of the CA1 circuit

As such, co-labeling studies here determine in which cell types PDE11 A4 is expressed in the aging brain and if a specific subtype segregates with cells showing increased expression of PDE11A4 around the cell body. If PDE11A4 continues to only be expressed in excitatory neurons during aging, then retrograde tracer studies using stereotaxically-delivered fluorogold are conducted to determine if this neuronal subpopulation segregates based on their projections. Of particular interest are anterior cingulate cortex, entorhinal cortex, and retrosplenial cortex as these brain regions show heightened activation in the PDE11A KO during retrieval of enhanced remote social aLTM. Nucleus accumbens (NAcc) are also of interest given that a subset of PDE11A4-expressing neurons project to NAcc and ventral CA1-NAcc projections are required for social aLTM.

Defining changes at the level of subcellular domain. To determine which subcellular domain constitutes the “filamentous structures” in which PDE11A4 accumulates with age, the slides collected above along are used with the previously published techniques to co-label for PDE11A4 and various markers, including those for axons, dendrites, glia, perineuronal nets, collagen, etc. Biochemical fractionation is conducted using DHIPP and VHIPP dissected from the second hemisphere of the rodents as well as anterior and posterior HIPP tissue samples from primates, with resulting fractions run on denaturing or native Western blots, all as per the published techniques. Fractionation experiments determine 1) if age-related increases in PDE11A4 protein expression occur preferentially in membrane vs. cytosolic vs nuclear fractions (denaturing blots; FIG. 9) and 2) if age-related increases in PDE11A4 protein expression are accompanied by increased PDE11A4 homodimerization (native blots). Increased PDE11A4 homodimerization, along with pS117/pS124, is expected to preferentially upregulate PDE11A4 expression in the VHIPP membrane as was observed in the preliminary study (FIG. 9). Together, experiments identifying where age-related increases in PDE11A4 expression occur at the level of subcellular compartments, cell types, and circuits and defines the potential intramolecular signals driving those compartment-specific effects.

Identifying mechanisms by which PDE11A4 expression patterns in old mice is restored to those of young mice. Previously, it was shown that expression of an isolated GAF-B domain (the domain required for PDE11A4 homodimerization) was sufficient to disrupt PDE11A4 homodimerization by acting as a negative sink. Further, it was shown that reduced levels of PDE11A4 homodimerization are associated with 1) reduced accumulation of PDE11A4, 2) a shifting of PDE11A4 from the membrane to the cytosol, and 3) reduced expression of PDE11A4 due to increased proteolysis. While not wishing to be bound by any particular theory, these studies suggest that age-related changes in PDE11 A4 expression/compartmentalization may be prevented/reversed by reducing levels of PDE11A4 homodimerization.

In vitro and in vivo experiments are conducted to determine if the isolated GAF-B domain can 1) reduce phosphorylation of PDE11A4 at S117/S124, 2) prevent/reverse the enhanced accumulation that is seen with the S117D/S124D mutations, and 3) prevent/reverse the increased membrane expression that is seen with S117D/S124D. In preliminary studies, it was shown that disrupting homodimerization in vitro reduces pS117/pS124 of PDE11A4^{WT} and restores trafficking patterns of St 17D/S124D to that of PDE11A4^{WT} (FIGS. 7D-7E). In vivo studies utilizing lentiviruses is conducted following the general approach described above. The isolated GAF-B domain (or control) are chronically expressed via lentiviral injection to the HIPP of old PDE11A WT mice to determine if disrupting PDE11A4 homodimerization is sufficient to restore aged PDE11A4 phosphorylation, expression, and trafficking patterns to those observed in young mice and, in so doing, prevent age-related decline of social aLTMs. Importantly, the lentiviruses express for at least 3 months, allowing for chronic manipulations. The GAB-B lentivirus have been obtained and it has been confirmed that it expresses in vivo. The effects of virally overexpressing S117A/S124A vs PDE11A4^{WT} (vs. a control lentivirus) are compared in HIPP of old PDE11A KO mice. In so doing, it is determined if preventing phosphorylation of S117/S124 is sufficient to block the accumulation of PDE11A4 that is seen with high levels of endogenous PDE11A4 expression (FIG. 8) or viral overexpression of PDE11A4^{WT}. Social aLTM using STFP is also assessed to determine if preventing phosphorylation of S117/S124 blocks the ability of PDE11A4 overexpression to impair social aLTM (FIGS. 5A-5E). Together, these studies help understand how/why age-related increases in PDE11A4 lead to age-related decline of social aLTM and, in so doing, identify novel therapeutic mechanisms by which the ectopic localization of PDE11A4 that occurs with age is addressed.

### Results:

Based on the consistencies of the findings in mouse, rat, and human tissue to date (FIG. 2B), it is believed age-related changes in PDE11A4 protein expression and compartmentalization are highly conserved across species. Based on the preliminary studies, it is anticipated that aging are associated with increased PDE11A4 expression and phosphorylation at S117/S124 in select compartments, notably the membrane fraction of VHIPP, select cell bodies in the superficial layer of CA1, and the filamentous structures that are most enriched in the subiculum. Although aging and S117D/S124D appear to regulate PDE11A4 in the opposite direction of disrupted homodimerization, it is do not believed aging are associated with increased homodimerization because in vitro studies show that S117D/S124D does not promote homodimerization. Nonetheless, it is anticipated that disrupting homodimerization remedies age-related deficits in PDE11A4 expression and compartmentalization by reducing phosphorylation of pS117/pS124 (FIG. 7D) and promoting proteolysis. Thus, it is expected that expression of the isolated GAF-B domain or S117A/S124A are sufficient to prevent age-related cognitive decline of social aLTMs.

It is not anticipated significant technical hurdles that prevents completion of the proposed experiments. In a non-limiting example, co-labeling studies are insufficient to identify the nature of the “filamentous structures,” despite the fact that there are a large number of validated antibodies that label axons, dendrites, etc.; and electron microscopy (EM) is used for study of ultrastructure. It has been determined the custom PDE11A4 antibody works with the EM fixative acrolein in immunohistochemistry, which is a strong predictor of an ability to work in a full EM protocol. In a non-limiting embodiment, tissue is labeled from EmGFP-PDE11A4 infected PDE11A KOs since overexpressed EmGFP-PDE11A4 also accumulates in filamentous structures in KOs and has been previously validated a GFP antibody in EM using EmGFP-PDE11A4 transfected COS-1 cells.

Westerns blots have been conducted and in situ hybridization in human tissue, so it is not anticipated unsurmountable difficulties in adapting the techniques for monkey tissue. In a non-limiting example, the NIA Primate Tissue bank tracks only age and general health of the monkeys, not their cognitive abilities. It was possible to detect age-related increases in PDE11A4 mRNA in human hippocampus without information regarding cognitive abilities (FIG. 2B), so it is believed the same are possible with monkey tissue. Thus, it is believed the proposed examination of this readily available monkey tissue is a worthwhile first step towards establishing the translatability of the findings, which is of particular concem when measuring age-related changes in the brain. Primates may show PDE11A4 expression in additional/alternative hippocampal subfields (e.g., DG) or cell types (e.g., inhibitory interneurons) than mice and rats, which is important given that location infers function. In this case, the functional consequences of virally expressing PDE11A^{WT} vs. a control virus in those additional subfields or specific cell types (i.e., by using a cell type-specific promoter) using WT mice are interrogated.

### Identify Molecular Mechanisms Driving Age-Related Increases in PDE11A4 Expression

As described above, the spatially restricted nature of PDE11A4 expression is maintained across the lifespan; however, steady-state levels of PDE11 A4 protein expression within the HIPP steadily increase. Increases in steady state levels of PDE11A4 protein could result from a number of mechanisms including increased rates of transcription and/or translation or increased stability of the transcript and/or protein. It has been shown previously that PDE11A mRNA expression is also increased in the aged rat brain and more recently discovered that PDE11A mRNA increases with age in the human hippocampus (FIG. 28). Thus, age-related increases in PDE11A4 protein expression appear to be driven, at least in part, by increases in PDE11A4 transcription and/or transcript stability.

In general, transcription of a given gene is controlled by a core promoter, promoter-proximal elements, as well as enhancers or silencers. Although the core promoter falls within 30 base pairs (bp) of the transcription initiation site (TIS) and the promoter-proximal elements fall within 200 bp of the TIS, enhancers and silencers can fall anywhere within 50 kB of the TIS. A number of studies have shown that a gene promoter is sufficient to drive transcription of a gene in a tissue-specific manner. Indeed transgenic technology makes great use of this fact by ligating the promoter of a tissue-specific gene to the coding sequence of a transgene in order to spatially restrict expression of that transgene to a desired tissue. Putative promoters have been described for hPDE11A4, hPDE11A3, and hPDE11A1 in the 1200 bps upstream of their respective TISs. Thus, to measure transcriptional activity of the mPde11a4 promoter, a lentivirus construct that uses the 1200 bps upstream of the mPde11a4 TIS to drive expression of the mCherry reporter (mPde11a4-mCherry) was developed. The decision to take a lentiviral approach is based on a prototype study by Chhatwal and colleagues. In a recent pilot study, infusion of a control Pgk-mCherry reporter resulted in mCherry expression in both CA1 and DG; however, infusion of the mPde11a4-mCherry reporter resulted in mCherry expression only in CA1 (FIG. 10). Although not wishing to be bound by any particular theory, this result suggests the tool faithfully tracks transcriptional activity of the mPde11a4 promoter, and so it is used it to compare PDE11A4 transcriptional activity in young vs old rodents.

### Experimental Approach.

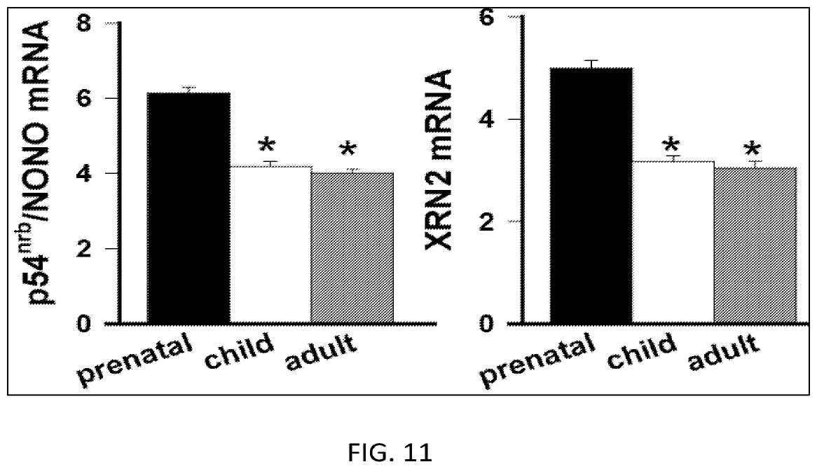

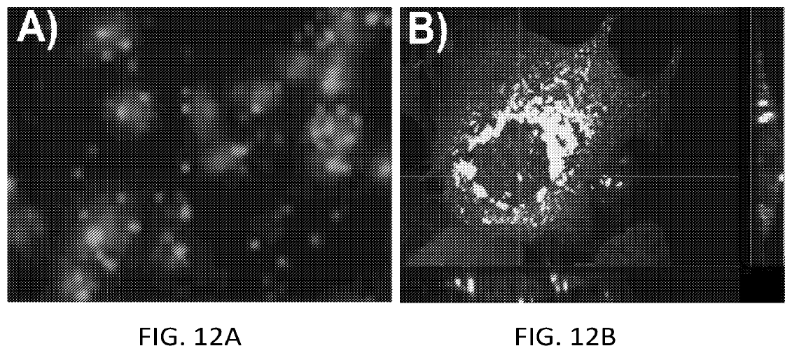

Assessing rates of PDE11A4 mRNA degradation by its known exoribonuclease (XRN2). First, in situ hybridization and Western blots are conducted on young vs. old HIPP tissue collected as described above to determine if age-related decreases in p54^{nrb}/NONO and XRN2 are observed in rodents and rhesus monkeys as they are in humans (FIG. 11). Immunoprecipitation are also conducted using total homogenates using methods described above. Antibodies are used against p54^{nrb}/NONO and XRN2 to perform pull downs and then RT-PCR is conducted for PDE11A4 mRNA. It is determined if old animals show less binding of p54^{nrb}/NONO and XRN2 to the PDE11A4 transcript than do young animals. Finally, nuclear and cytoplasmic RNAs are isolated from young and aged rodents using the Ambion Paris system according to manufacturer's instructions (Life Technologies) and, again, RT-PCR for PDE11A4 mRNA are conducted. This allows for determination if aging is associated with heightened accumulation of PDE11A4 mRNA in the nucleus vs. the cytosol, which indicates impaired degradation by the p54^{nrb}/NONO-XRN2 complex. In the event of finding a relative increase in nuclear PDE11A4 mRNA expression, brain sections collected as described above are used to conduct confirmatory in situ hybridization experiments using RNAscope probes (ACD). RNAscope probes enable single molecule-level resolution (see FIG. 12A) and when combined with confocal imaging (FIG. 12B), enable the qualitative assessment of PDE11A mRNA expression in the nucleus vs. the cytosol.

### Results

It is expected to find increased mCherry expression in old vs young rodents only when they are injected with the mPde11a4-mCherry into hippocampal subfields that show increased PDE11A4 mRNA expression. In a non-limiting example, if the constitutive Pgk-mCherry reporter also shows increased expression in the aged hippocampus, then this result suggests a more global upregulation of transcriptional activity, or a potential indirect effect of the mCherry itself, as opposed to a selective upregulation of PDE11A4 transcriptional activity. In a non-limiting example, the finding that aging is associated with increased PDE11A4 transcription or transcript stability does not rule out the possibility that aging is also associated with increased rates of translation or protein stability, and the possibility of age-related reductions in sumoylation or ubiquitination of PDE11A4 is further examined.

The pilot data suggests the 1200 bps upstream of the PDE11A4 TIS is sufficient to control the transcription of mPde11a4 in terms of its spatial distribution, but it is possible that aging also/alternatively could influence rates of transcription by differentially engaging enhancer or silencing motifs within 50 kB of the TIS. In a non-limiting example, to identify cis-acting elements beyond the promoter region that might control PDE11A4 transcription, a BAC transgenic approach is adopted as previously described by Koppel and colleagues. BAC transgenic mice are generated that express a fluorescent protein under the control of a minimal promoter coupled with various combinations of PDE11A4 exons, introns, 5′ upstream sequences, and 3′ downstream sequences.

Further, exoribonucleases are not the only mechanism regulating transcript stability; microRNAs, along with other noncoding RNAs, play an important role as well. In another non-limiting example, the focus is to identify non-coding RNAs that regulate PDE11A4 expression. Of primary interest are miR-375. This is one of only 4 microRNAs that is predicted by TargetScan to target the PDE11A4 transcript. Interestingly, miR-375 expression decreases with age in the mouse brain, which is consistent with the observed age-related increases in PDE11A4.

Statistical Analyses: Power analyses are conducted post hoc to confirm that n=10/sex/group provides sufficient power for in vivo studies and n=4 biological replicates/group (×3 experiments) provides sufficient power for cell culture experiments (as they have in the past). In Western blot experiments, samples generally must span multiple blots (particularly in fractionation studies). Therefore, to account for non-specific technical differences across blots (e.g., transfer or antibody binding efficiency, film exposures etc.), all biochemical data are normalized as a fold change of the control group on a given blot, as have been previously described. All datasets meeting normality and equal variance assumptions are tested by parametric statistics. Those datasets not meeting these assumptions are tested by nonparametric statistics. In general, data are analyzed by multifactorial ANOVAs or by repeated measure ANOVAs where appropriate to account for multiple comparisons. For example, in vivo data are analyzed for effects of sex, genotype, lentiviral treatment (in addition to assay-specific factors, such as food type). Statistical outliers (>2 standard deviations from the mean) are dropped from analyses, as previously described. Significant ANOVAs are followed by Student Newman-Keuls post hoc tests, with significance determined as P<0.05. Data in figures are plotted as means±SEMs.

### Data Rigor

The rationale is based on both mouse and human data with follow-up with studies in mice, rats and monkeys, which strengthens the rigor of this proposal. Further, the primary findings have been replicated across multiple cohorts of mice. Achieving robust and unbiased results: Genetically modified mice are genotyped a priori to enable proper counterbalancing of experimental run lists; however, experimenters are blind to genotype at the time of data collection. Genotypes are then reconfirmed post death by Western blot or in situ hybridization. Physical parameters are counterbalanced across subjects (e.g., which is the “trained” spice and which is “novel”). Biological Variables: both males and females were tested for effect of sex.

Since cohorts of mice are aged up, these experiments largely conducted in parallel across years 1-5. One exception is that some experiments slightly lag behind as species selection depends on the outcome of in situ hybridization studies described herein.

These innovative studies provides much needed insight into the fundamental mechanisms of age-related cognitive decline as well as the function and regulation of PDE11A4, including establishing whether PDE11A represents a therapeutic target not only for preventing age-related decline of social aLTMs but also for reversing deficits. In a non-limiting example, the effects of excessive PDE11A4 on acquisition vs. consolidation. vs. retrieval of remote social aLTMs are studied as well as those aimed at understanding the system-level mechanism by which altered cyclic nucleotide signaling in the VHIPP can impair social aLTMs (e.g., by compromising the integrity of neuronal ensembles encoding the memory engram in the hippocampus vs. cortex). Studies also determine if excessive PDE11A4 is problematic simply by virtue of increased steady state levels that are ubiquitously distributed or by virtue of a discretely localized upregulation or even an ectopic expression of PDE11A4. In a non-limiting example, the effect of modulating PDE11A4 function within specific cell-types (e.g., excitatory vs. inhibitory) or sub-region (e.g., subiculum vs. CAT) is examined. In addition, the upstream signaling events are delineated, including the specific kinases that lead to age-related increases in the phosphorylation of PDE11A4. The fact that aging changes the compartmentalization of PDE11A4 provides a biologically-relevant framework for exploring the functional impact of PDE11A post-translational modifications and protein-protein binding events. Defining the signals that control age-related changes in PDE11A4 trafficking enables a more sophisticated targeting of compartment-specific localizations of the enzyme as opposed to overall catalytic activity, which is preferred since eliminating all PDE11A catalytic activity influences social preferences and impairs recent social LTMs. Studies also identify the mechanism(s) by which PDE11A4 expression is upregulated with age. In a non-limiting example, if age-related increases in PDE11A4 expression are driven by increased transcription, additional studies can be performed to seek to identify the transcription factors responsible. Identifying the mechanism driving age-related increases in PDE11A4 open up new avenues for therapeutically restoring normal levels of expression. Overall, the results provide proof of principle for pursuing PDE11A4 as a novel therapeutic target. PDE11A is a highly druggable enzyme and it is positioned to selectively control cyclic nucleotide signaling in a molecularly-defined circuit that specifically regulates social LTMs, without affecting signaling elsewhere. This may relieve age-related impairments in aLTM without causing unwanted side effects. Importantly, PDE11A exhibits all properties the pharmaceutical industry believes an ‘ideal’ drug target should have. The fact that PDE11 A is a realistic candidate for drug development increases the value of understanding its biological function.

### Example 2: Role of Cyclic Nucleotide Signaling in Age-related Decline of Social Memories

This Example describes studies where it is hypothesized that age-related increases in HIPP PDE11A4 occur in a compartmentalized manner and impair social aLTM.

Determine if blocking age-related increases in PDE11A4 can prevent and/or rescue age-related impairments in social aLTM. It is not known if age-related increases in hippocampal PDE11A4 expression reflect a physiological breakdown or an attempt at protective compensation. In studies, PDE11A WT mice show age-related impairments in social transmission of food preference (STFP) aLTM but PDE11A KO mice do not. Further, mimicking the condition of an old WT by overexpressing PDE11A4 in the hippocampus of PDE11A KO mice impairs STFP aLTM. It is hypothesized that preventing/reversing age-related increases in PDE11A expression are sufficient to prevent/reverse age-related impairments in social aLTM. An innovative conditional transgenic system is used to prevent, reverse, or mimic age-related increases in HIPP PDE11A4 and determine the effect on social aLTM vs non-social aLTM and social/nonsocial rLTM, neural circuit activation, as well as biochemical endpoints widely reported to change with age (e.g., CREB). These studies determine if PDE11A represents a therapeutic target not only for preventing age-related decline of social aLTMs but also for reversing deficits.

Identify the circuit, cell-type and subcellular domain where PDE11A4 is upregulated with age and the signal driving this compartmentalized effect. Previously it was shown that PDE11A4 protein expression increases with age in HIPP lysates. It is possible that this excess PDE11A4 may not be globally distributed, but rather discretely localized or even ectopically expressed in cell types or subcellular domains that are normally void of PDE11A4. In studies, biochemical fractionation shows PDE11A4 increases with age preferentially in membrane vs cytosol and nucleus. Immunofluorescence shows PDE11A4 ectopically accumulates in filamentous structures in the aged HIPP. which are rarely seen in young HIPP. Further, this pool of accumulated PDE11A4 is uniquely phosphorylated at serines 117 and 124. In vitro, phosphomimic mutation of S117 and S124 drives accumulation of PDE11A4, while phosphoresistant mutations or blocking homodimerization reduces this accumulation.

In vivo studies are conducted to 1) quantify circuit, cell-type, and subcellular domain-specific age-related increases in PDE11A4 expression/phosphorylation in rodents and primates (for translatability) and 2) determine if blocking phosphorylation of S117/S124 or disrupting PDE11A4 homodimerization in mice can prevent/reverse ectopic trafficking of PDE11A4 and social aLTM deficits. It is hypothesized that age-related increases in pS117/pS124 drive ectopic localization of PDE11A4, contributing to age-related decline. Outcome: Defining the signals that control age-related changes in PDE11A4 trafficking enables a more sophisticated targeting of compartment-specific localizations of the enzyme as opposed to its total catalytic activity.

Identify molecular mechanisms driving age-related increases in PDE11A4 expression. Age-related increases in steady state levels of PDE11A4 protein could be driven by an increase in transcript/protein generation and/or transcript/protein stability. In studies, it was found that PDE11A4 mRNA is increased in aged vs. young rat HIPP and demented vs. nondemented human HIPP. It is hypothesized that age-related increases in PDE11A4 protein expression are driven by increased rates of transcription and/or transcript stability. The novel PDE11A4-promoter reporter is used to measure transcription in HIPP of aged vs. young mice. In addition, rates of PDE11A4 mRNA degradation are assessed by its known exoribonuclease (XRN2). By identifying the mechanism driving age-related increases in PDE11A4 new avenues are open for therapeutically restoring normal levels of expression.

These innovative studies provides much needed insight into PDE11A function/regulation and broaden the understanding of age-related cognitive decline via the interrogation of a very specific molecularly-defined circuit that appears to selectively regulate social memories and their decline.

### Example 3: Biologic That Disrupts PDE11A4 Homodimerization in Hippocampus CA1 Reverses Age-Related Proteinopathies in PDE11A4 and Age-Related Decline of Social Memories

This Example describes studies demonstrating that age-related increases in phosphodiesterase 11 A (PDE11A), an enzyme that degrades 3′,5′-cAMP/cGMP and is enriched in the ventral hippocampal formation (VHIPP), drive age-related cognitive decline (ARCD) of social memories. In the VHIPP, age-related increases in PDE11A4 occur specifically within the membrane compartment and ectopically accumulate in filamentous structures termed ghost axons. Previous in vitro studies show that disrupting PDE11 homodimerization by expressing an isolated PDE11A-GAFB domain that acts as a “negative sink” for monomers selectively degrades membrane-associated PDE11A4 and prevents the punctate accumulation of PDE11A4. Therefore, it was determined if disrupting PDE11A4 homodimerization in vivo via the expression of an isolated PDE11A4-GAFB domain would be sufficient to reverse 1) age-related accumulations of PDE11A4 in VHIPP ghost axons and 2) ARCD of social memories. Indeed, in vivo lentiviral expression of the isolated PDE11A4-GAFB domain in hippocampal CAT reverses the age-related accumulation of PDE11A4 in ghost axons, reverses ACRD of social transmission of food preference memory (STFP), and improves remote long-term memory for social odor recognition (SOR) without affecting memory for non-social odor recognition. In vitro studies suggest that disrupting homodimerization of PDE11A4 does not directly alter the catalytic activity of the enzyme but may reverse age-related decreases in cGMP by dispersing the accumulation of the enzyme independently of other intramolecular mechanisms previously established to disperse PDE11A4 (e.g., phosphorylation of PDE11A4 at serine 162). Although not wishing to be bound by any particular theory, altogether these data suggest that a biologic designed to disrupt PDE11A4 homodimerization may serve to ameliorate age-related deficits in hippocampal cyclic nucleotide signaling and subsequent ARCD of remote social memory.

Therefore, it is sought to determine if a biologic that decreases PDE11A4 homodimerization will be sufficient to reverse age-related accumulations of PDE11A4 in ghost axons and rescue ARCD of social memories.

### Methods

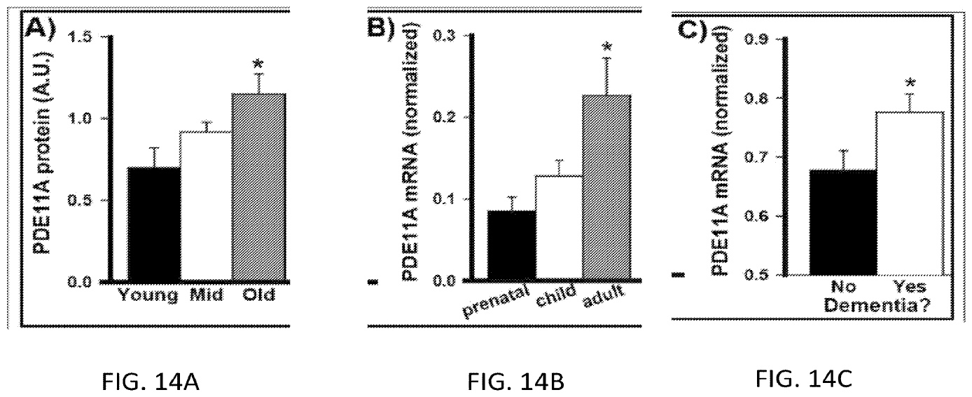

Subjects. C₅₇BL6/J mice were originally obtained from Jackson Laboratory (Bar Harbor, ME) and the line was maintained at the University of South Carolina. The Pdella mouse line obtained from Deltagen (San Mateo, CA) was maintained on a mixed C₅₇BL6 background (99.8% multiple C₅₇BL/6 substrains, 0.2% 129P2/OlaHsd). Pdella mice were bred at the University of South Carolina in heterozygous (HT)×HT trio-matings. Same-sex wild-type (WT), heterozygous (HT), and knockout (KO) littermates were weaned and caged together to total 3-5 mice/cage. It is not believed litter effects are driving findings here due to each dataset reflecting multiple cohorts born and tested at different times. Further, a litter of mice normally contributes only 1-2 mice/genotype and parents contribute two litters at most to a cohort. While both males and females were used in experiments, analyze for sex effects (see figure legends for specific n's/sex/group/experiment). In these studies, young mice were defined as 2-6 months and old mice were defined as 18-22 months. “Young” mice included both young Pdella WT mice surgerized alongside old Pdella WT mice (i.e., receiving bilateral injections of mCherry lentivirus to the dorsal and ventral hippocampi) and unsurgerized young C₅₇BL6/J mice that were used as an internal control for the assays (FIGS. 14A-14B). Since no obvious differences were found between groups of young surgerized Pde11a WT mice and young unsurgerized C₅₇BL6/J mice, the data from these 2 subgroups were subsequently combined into a singular “young” (FIGS. 14C-14G). All mice used in experiments were generally healthy throughout the duration of testing. Gross pathology was not conducted but mice were routinely assessed by husbandry, veterinary, and laboratory staff. Mice with lethargy, altered gait, signs of malnutrition or dehydration, noticeable tumors >1 cm, were removed from study and euthanized.

The effects of healthy aging were studied, and therefore if upon brain dissection evidence was found of an anatomical abnormality (e.g., a pituitary tumor), the animals was excluded from the study (note: no occurrences in this study). A 12:12 light:dark cycle and ad lib access to food and water were provided. Experiments were conducted in accordance with the National Institutes of Health Guide for the Care and Use of Laboratory Animals (Pub 85-23, revised 1996) and were fully approved by the Institutional Animal Care and Use Committee of the University of South Carolina and the University of Maryland, Baltimore.

Tissue Collection: Mice were euthanized (during light cycle) via rapid cervical dislocation and brains were immediately collected and flash frozen on 2-methylbutane sitting on dry ice. Brain tissue was then stored at −80° C. until cryosectioning at 20 μm to verify viral expression.

Social Transmission of Food Preference. Subjects' access to food was restricted the two days prior to testing to one hour per day. The day prior to testing, all mice were placed in a clean home cage and given access to plain powdered chow packed into a glass jar. The following day, the designated “demonstrator mouse” was individually placed in a clean home cage and fed powdered chow flavored with a household spice (e.g. basil vs. ginger, thyme vs turmeric, mint vs cardamom, orange vs anise, basil vs thyme). After one hour, the “demonstrator mouse” was returned to the original home cage where the “observer” cage mates were allowed unrestricted access to the demonstrator for 15 minutes. It is during this time that the observers make an association between the social pheromones in the breath of the demonstrator and the non-social odor (household spice). Recent and remote long-term memory were assessed 24 hours or 7 days after training, respectively. At that time, the observer mice were individually placed in clean home cages and given access to two flavored/powdered chows for 1 hour. One flavored chow contained a novel spice and the other contained the spice that the demonstrator was given. The amount of food eaten was measured by an experimenter blind to treatment. All mice had to meet minimum inclusion criteria including eating at least 0.25 grams of food. Cohorts were able to be trained/tested at multiple time points using different spice combination to reduce the total number of mice used and we have shown that this does not confound interpretation of the data. Observer mice eating more food containing the familiar spice (i.e., the spice on their demonstrator's breath) versus the novel spiced food constituted memory (preference ratio: familiar-novel/familiar+novel).

Odor Recognition. Subjects were allowed to habituate to 1″ round wooden beads (Woodworks) for at least seven days prior to testing by placing several beads in the subjects' home cages. For social odor recognition (SOR), the wooden beads take up the scent of the cage of each strain used (e.g., C₅₇BL/6J Jax #000664, BALB/cJ Jax #000651, 129S6/SvEv Taconic #129SVE) and are used as the social odor in testing. For non-social odor recognition (NSOR), the wooden beads were placed in a bag containing bedding saturated with a household spice (e.g., marjoram, cumin, etc.) for at least 7 days. Training for SOR and NSOR consisted of a habituation trial with 3 beads from the subject's home cage, followed by two training trials that included 2 home-cage beads and 1 novel-scented bead. Recent and remote long-term memory were assessed 24 hours or 7 days after training, respectively. During SOR, mice were tested with one home cage bead, one bead from the trained donor strain (familiar), and one bead from a second donor strain (novel). During NSOR, mice were tested with only two beads, one scented with the training spice and one a novel spice. The designation of which scent was “novel” within a given testing trial and the location of the novel scent (i.e., left versus right) was counterbalanced across subjects. Mice were given two minutes to investigate the beads and the amount of time spent on each was manually scored by an experimenter blind to treatment and bead. It was previously determined that infusion of even a negative control lentivirus into the hippocampus reverses the recent long-term memory impairment observed in Pde11a KO mice 24 hours after training. Therefore, 24-hour memory following injection of the isolated GAF-B domain was not tested as the results would not be interpretable. All mice met minimum inclusion criteria of spending a minimum of 3 seconds in total sniffing the beads. Spending more time investigating the novel vs familiar scent constituted memory (preference ratio: novel-familiar/novel+familiar).

Stereotaxic Surgeries. Stereotaxic surgeries and injections were performed using a NeuroStar motorized stereotaxic, drill, and injection robot (Tubingen, Germany). Mice were anesthetized with a steady flow of oxygen and isoflurane. The mice were induced at 3% isoflurane and maintained at 1-1.5%. Lack of reflexes was verified and the scalp was then shaved and cleaned with betadine. A small incision was made in the scalp and the skull was cleared with sterile saline. Cotton swabs were again used to visualize the skull and locate Bregma. Using the robotic drill, small holes were made above the dorsal and ventral hippocampi as per the following coordinates relative to Bregma: dCA1 AP, −1.7, dCA1 ML, +/−1.6, vCA1 AP, −3.5, vCA1 ML, +/−3.0. At a speed of 10 mm/see, a Hamilton syringe (custom needle #7804-04: 26s gauge, 1″ length, 25 degree bevel) was then then placed to the following depths relative to Bregma: dCA1 DV, −1.3, vCA1 DV, −4.4. After a thirty second pause following the needle movement, the injection robot was used to inject 2 μ1 of lentivirus at 0.167 μl/minute. Following injection completion, the experimenter waited two minutes to allow the lentivirus to diffuse away from the needle and the needle was raised at the same speed. After all injections were complete, pronged tweezers were used to close the scalp and secured using glutures. Buprenorphine in sterile saline at a dose of 0.1 mg/kg was injected IP for pain management. For recovery, the mouse was placed on a warm Deltaphase pad and allowed to recover until moving normally and posturing upright. Mice were allowed at least 2 weeks of recovery in grouped home cages prior to behavioral testing.

A lentivirus carrying an mCherry-tagged PDE11A4-GAFB served to disrupt PDE11A4 homodimerization, while an mCherry-only virus was used as a negative control. A lentiviral construct was used here in order to compare to previous studies examining the effects of overexpressing PDE11A4 in vivo, which required the use of a lentiviral cassette due to the large size of PDE11A4. The viruses were made on an “SPW” backbone that drives expression using the phosphoglycerate kinase 1 (PGK) promoter, which in theory is a ubiquitous promoter and yet is taken up preferentially by neurons. It was previously shown that the isolated GAF-B construct disrupts PDE11A4 homodimerization by binding to PDE11A4 and triggering proteolytic degradation. For reasons that are not well understood, the GAF-B construct degrades PDE11A4 more significantly in the membrane versus cytosolic fractions. The lentiviruses were prepared and diluted in 0.2 M sucrose/42 mM NaCl/0.84 mM KCl/2.5 mM Na2HPO4/0.46 mM KH2PO4/0.35 mM EDTA and the original titers were as follows: mCherry, 7.37×10E10/mL; GAF-B, 1.82×10E10/ml. Pilot studies using wide-field fluorescent microscopy determined diluting the mCherry-only virus to one-third the original concentration equated comparable mCherry expression between viruses, and so this concentration was used in experiments. While high viral expression was found throughout CA1 of hippocampus in all mice, a subset of mice exhibited some viral expression in dentate gyrus, CA2, and/or CA3. This ectopic viral expression does not appear to affect the results given that 1) it is found in both the mCherry-only and mCherry-GAFB groups and 2) PDE11A4 expression does not emanate from the dentate, CA2 nor CA3.

It was found that cells in proximal CA1 (closer to CA3) relative to distal CA1 (closer to subiculum) more readily took up the virus, which mirrors the preferential distribution pattern of endogenous PDE11A4 across CA1. Importantly, no gross cellular toxicity or morphological damage was found with either virus, and animals were healthy following surgery. The accuracy of the injection and expression of the viral construct was verified by direct visualization of raw florescence and/or following immunofluorescence (see below).

Immunofluorescence. A cryostat was used to section fresh-frozen mouse brains at m. Sections were thaw-mounted onto slides and dried briefly at room temperature before storing at −80° C. until processing. The slides were fixed in 4% paraformaldehyde in phosphate-buffered saline (PBS) for 20 minutes. After fixation, 3×10 minute washes with PBS and 3×10 minute washes with PBT (phosphate-buffered saline/0.4% BSA/0.3% Triton-X 100) were performed to reduce background. Primary antibodies were combined in a tube with PBT at validated and optimized concentrations: PDE11A4 (Aves custom PDE11A4 #1at 1:10.000; Fabgennix PPD11A-140AP at 1:1000; Fabgennix PPD11A-150AP at 1:500) and mCherry (ThermoFisher #PA534974 at 1:1000; Invitrogen #M11217 at 1:500; PhosphoSolutions #1203-mCherry at 1:10,000). Multiple PDE11A antibodies were utilized to discern diffuse expression versus accumulations of PDE11A4. While the PDE11A4 #1 antibody labels all PDE11A4 and, thus, detects both diffusely localized and accumulated PDE11A, the PDE11A4-140 and 150 antibodies specifically labels PDE11A4 phosphorylated at serines 117 and/or 124 and, thus, only detects PDE11A4 accumulated in ghost axons. To limit non-specific labeling, mCherry antibodies hosted in a rodent species (Invitrogen and PhosphoSolutions), were pretreated with anti-Mouse FabFragments (0.15 mg/ml; Jackson Immunoresearch #715-007-003) in PBS for 2 hours, followed by 3×10 minute washes in PBT prior to adding primary antibody. Primary antibody solution was added over brain sections and the slides were kept level at 4° C. overnight. Primary antibodies was removed using 4×10 minute washes in PBT. For optimal labeling, PDE11A4 secondary antibody (Alexafluor 488 AffiniPure Donkey Anti-Chicken, 1:1000, Jackson Immunoresearch #703-545-155) was applied first to the slides for 90 min at room temperature. The secondary was washed off using 3×10 minute washes with PBT. Next, the mCherry secondary (Alexafluor 594 AffiniPure species-specific, 1:1000, Jackson Immunoresearch) was repeated for the same time and conditions. Finally, 3×10 minute washes with PBT were used to clear the slides of any remaining secondary and the slides were briefly dip-rinsed in PBS to remove Triton. The slides were wiped dry along back, sides, and edges and mounted using DAPI Fluoromount-G (Southern Biotech, #0100-20). PDE11A4-filled structures were quantified by an experimenter blind to treatment with images captured using Leica Application Suite (LASX) software and a Leica DM5000 B florescent microscope. Brightness, histogram stretch, and/or contrast of images was adjusted for graphical clarity.

Cell culture and transfections. COS-1 (male line), HEK293T (female line), and HT-22 (sex undefined) cell culture and transfection were performed as previously described [1]. While kept in t-75 flasks, cells were grown in Dulbecco's Modified Eagle Medium (DMEM) (GIBCO; Gaithersburg, MD, USA), 10% fetal bovine serum (FBS) (GE Healthcare Life Sciences; Logan, UT, USA), and 1% Penicillin/Streptomycin (P/S) (Mediatech, a Corning subsidary; Manassas, VA, USA). Cells were incubated at 37° C./5% CO₂ and passaged using TrypLE Express (GIBCO; Gaithersburg, MD, USA) as a dissociation agent once 70-90% confluent. One day before transfection began, cells were plated along with DMEM+FBS+P/S in either 24-well plates for imaging or 100 mm dishes for biochemistry. The day of transfection, Optimem (GIBCO) replaced the media. According to manufacturer protocol (ratio of 3.75 ug DNA plus 10 uL lipofectamine per 10 mLs of media), cells were transfected with Lipofectamine 2000 (Invitrogen; Carlsbad, CA). ~19 hours post-transfection (PT), the Optimem/Lipofectamine solution was removed and replaced with DMEM+FBS+P/S. Normally, cells grew for five hours before sample processing, which meant they were harvested about 24 hours following transfection. Additionally, cells were sporadically tested for mycoplasma, with negative results always obtained. For assessment of subcellular trafficking, paraformaldehyde (4%) in PBS was used to fix the cells for fifteen minutes, after which they were kept in PBS until imaging. Images were captured using NIS-Elements BR-2.3 (Nikon, Tokyo Japan) on a CoolSNAP EZ CCD camera (Photometrics, Tucson AZ) mounted on an inverted Leica (Wetzlar DE) DMIL microscope with a Fluotar 10×/0.3 ∞/1.2 objective.

All images pertaining to an experiment were quantified by an experimenter blind to treatment using the same computer within the same position in the room, the same lighting conditions, and the same percent zoom. Images were loaded onto a gridded template to facilitate keeping track of count locations within the image, and an experimenter scored each image box by box, with cells along the top and left edges of the image as a whole not included to follow stereological best practices. Images were quantified in a counterbalanced manner such that 1 picture from each condition was evaluated before moving onto a 2nd image from that condition. The experimenter classified cells as exhibiting either cytosolic-only labeling or punctate labeling (with or without cytosolic labeling present), with data expressed as the % of the total number of labeled cells that exhibited punctate labeling.

Biochemical Fractionation and Western Blotting. Biochemical fractionation was performed to obtain cytosolic and soluble membrane fractions. Cell were mixed with ice cold fractionation buffer (20 mM Tris-HCl, pH 7.5; 2 mM MgCl2; Thermo Pierce Scientific phosphatase tablet #A32959 and protease inhibitor 3 #PO₀₄₄) and sonicated. First, a low-speed spin (1000×g) removed cellular debris and the supernatant from this spin was transferred to a new tube. Next, a high speed spin (89,000×g) was performed to obtain the membrane (pellet) and cytosolic (supernatant) proteins. After being suspended in fractionation buffer with 0.5% Triton-X 100 to solubilize the protein, the membrane pellet was sonicated and subjected to a high-speed spin (60,000×g). The supernatant containing all membrane proteins was then placed in a new tube. Samples were nutated for at 4° C. for 30 minutes. A second high-speed spin (60,000×g) was done for 30 minutes to separate the soluble membrane (supernatant) from the insoluble membrane (pellet). The soluble membrane sample was then transferred to a clean tube and used for western blot (see below). A DC Protein Assay kit (Bio-Rad; Hercules, CA, USA) was used to determine protein concentrations by which total protein was equalized across samples. Samples were stored at −80° C. until used in Western blotting. For Western blotting, 10 μg of protein was loaded onto 12% NuPAGE Bis-Tris gels (Invitrogen, Waltham MA) and run at 180 volts. After about one hour, the proteins remaining in the gels were transferred onto 0.45 um nitrocellulose membrane using 100 mA for two hours. Following transfer, tris-buffered saline with 0.1% tween20 (TBS-T) was used to wash membranes. Membrane were cut into multiple strips to probe for multiple antibodies if needed. The membranes were blocked using either 5% milk or Superblock (PBS) Blocking Buffer (ThermoFisher, Cat #37515). Primary antibody was applied overnight at 4° C. for PDE11A (Fabgennix PD11-101 at 1:500). The following day, membranes were washed with TBS-T (4×10 minutes). Secondary antibody (Jackson Immunoresearch Anti-Rabbit, 111-035-144; 1:10 000), was applied at room temperature for one hour. Finally, the membrane was washed in TBS-T (3×15 minutes). Chemiluminescence (SuperSignal West Pico Chemilumiscent Substrate; ThermoScientific, Waltham MA) was captured using film and multiple exposures were taken to ensure densities were within the linear range of the film. ImageJ was used to quantify optical densities. Each blot was normalized to a control condition (e.g., WT) to account for any technical variables (film exposure, antibody signal-noise, variance in chemiluminescence, etc.).

PDE assay. cAMP- and cGMP-PDE catalytic activity were measured. The assay was validated in vitro using HT-22 cells (a mouse hippocampal cell line). Buffer containing 20 mM Tris-HCl and 10 mM MgCl2 was used to harvest cells and kept on ice until ready to use. PDE activity was measured using 50 μl of sample and 50 μL of [3H] cAMP (Perkin Elmer, NET275) or cGMP (Perkin Elmer, NET337) and incubated for 10 minutes. After incubation, 0.1 M HCl was added to quench the reaction, followed by 0.1M Tris to neutralize the reaction. 3.75 mg/mL snake venom (Crotalus atrox, Sigma V-7000) was then added to complete the reaction and the mixture was incubated at 37° C. for 10 minutes. Samples were put into 5′polystyrene chromatography columns with coarse filters (Evergreen, 208-3383-060) containing DEAE Sephadex A-25 resin (VWR, 95055-928). The columns were equilibrated in high salt buffer (20 mM Tris-HCL, 0.1% sodium azide, and 0.5 M NaCl) and low salt buffer (20 mM Tris-HCL and 0.1% sodium azide). The reactions were then run down the equilibrated columns. Following four washes with 0.5 ml of low salt buffer, 4 ml of Ultima Gold XR scintillation fluid (Fisher, 50-905-0519) was added to the eluate and mixed thoroughly. A Beckman-Coulter liquid scintillation counter Beckman LS 6000) was used to read counts per minute (CPM). As an assay control, two reactions free of sample lysate were ran in parallel to account for background activity and could then be subtracted from the sample CPMs. Total protein levels were quantified using the DC Protein Assay Kit (Bio-Rad, Hercules, CA) as described above, and CPMs were then normalized to the total amount of protein in each sample.

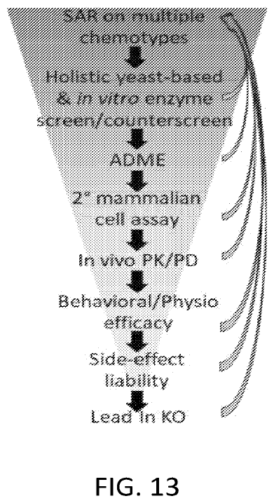

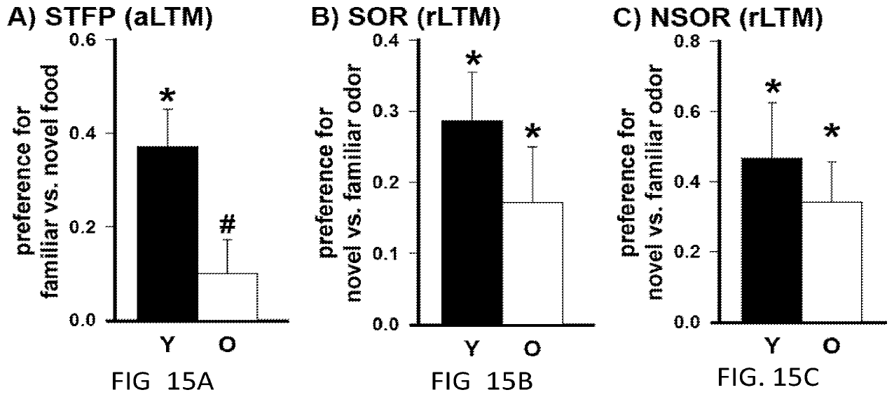

Data Analysis. Data was collected by investigators blind to treatment and experiments were designed to counterbalance technical/biological variables. Outliers more than 2 standard deviations away from the mean were removed prior to analyses. Outliers removed/total n: FIG. 13G, 1/23; FIG. 14A, 2/18; FIG. 14B, 1/22; FIG. 14E, 4/49; FIG. 15A, 1/18; FIG. 15B, 1/18; FIG. 15C, 3/45; FIG. 15G, 2/20; FIG. 15I, 9/112; FIG. 15J, 3/36; FIG. 15K, 3/36. Data were analyzed for effect of genotype, behavioral measure (e.g., bead or food), and treatment. Parametric statistical analyses were run on SigmaPlot 11.2 (San Jose, CA, USA) including ANOVA (F), Student's t-test (t), and one-sample t-test (t) when datasets met assumptions of normality (Shapiro-Wilk test) and equal variance (Levene's test). To offset the possibility of a Type I error associated with multiple comparisons, a false-rate discovery (FDR) correction was applied to P-values or one-sample t-tests within an experiment. If analyses failed normality and/or equal variance, nonparametric Kruskal-Wallis ANOVA (H) or Mann-Whitney rank sum test (T) were used instead. Student-Newman-Keuls or Dunn's test were performed for Post hoc analyses. Significance was defined as P<0.05.

### Results

Disrupting PDE11A4 homodimerization selectively decreases PDE11A4 expression in a compartment-specific manner and is sufficient to decrease PDE11A4 accumulations in ghost axons that occur with aging. It was previously found that ventral hippocampal PDE11A4 expression increases with age in both mice and humans and that these age-related increases accumulate specifically in the membrane compartment and within filamentous structures termed “ghost axons”. Additionally, it was found in vitro that disrupting PDE11A4 homodimerization by expressing an isolated GAF-B binding domain that acts as a negative sink (FIG. 13B) leads to proteolytic degradation specifically of membrane-bound PDE11A4 and reduces the accumulation of PDE11A4 into punctate structures. Therefore, the studies sought to determine if disrupting PDE11A homodimerization in vivo would be sufficient to reverse age-related increases in PDE11A4 expression and accumulation. To do this, lentiviruses were used (FIG. 13A) containing either mCherry alone (i.e., negative control) or an mCherry-tagged isolated GAF-B domain that disrupts PDE11A4 homodimerization (FIG. 13B). These lentiviruses were stereotactically injected bilaterally into the CAT field of dorsal and ventral hippocampi of old Pdella WT mice, since this is the field where PDE11A4 regulates social learning and memory. All mice demonstrated mCherry signal in dorsal and ventral CAT, with a subset of mice exhibiting expression in neighboring hippocampal sub-regions (e.g., dentate gyrus, CA3, CA2) that do not express PDE11A4 (FIG. 13D). While mCherry-treated mice exhibit a uniform pattern of PDE11A4 expression across stratum radiatum, stratum pyramidale and stratum oriens of CAT, GAF-B treated mice exhibit a compartment-specific decrease in PDE11A expression, with reduced expression in the distal segment of stratum radiatum and stratum oriens relative to stratum pyramidale (FIG. 13E). Consistent with the fact that the isolated GAF-B domain reduces PDE11A4 protein expression in the membrane compartment, it was found that disrupting homodimerization of PDE11A4 reduced age-related increases in so-called “PDE11A4 ghost axons” (i.e., filamentous structures harboring age-related accumulations of PDE11A4) (FIG. 13F). The ability of the GAF-B construct to reduce the accumulation of PDE11A4 in ghost axons was confirmed using two different PDE11A4 antibodies on two different sets of slides (FIG. 13G). Without wishing to be bound by any particular theory, all together these data suggest disruption of PDE11A homodimerization in vivo is sufficient to reverse age-related increases in PDE11A4 protein expression and ectopic accumulation.

Disrupting PDE11A4 homodimerization in the hippocampus of old mice is sufficient to reverse age-related decline of remote long-term social memory. It was found that while Pde11a WT mice suffer from ARCD of remote long-term social associative memories, Pde11a KO mice do not. Unfortunately, this protection of remote LTM 7 days after training comes at the expense of not being able to access recent LTM 24 hours after training. Therefore, it was determined if disrupting homodimerization of PDE11A4 using the isolated GAF-B domain is sufficient to induce a transient amnesia in old Pde11a WT mice that ultimately reverses ARCD of remote long-term social associative memories.

Social associative memory was measured using social transmission of food preference (STFP), an assay where mice form an association between a non-social odor (i.e., a household spice) and a social odor (i.e., pheromones in their cage mate's breath), with the memory of that association indicating a food with that scent is safe to eat. Mice treated with the GAF-B domain showed no recent LTM for STFP but did show remote LTM for STFP on par with that of young adult mice; whereas, mice treated with mCherry alone showed ARCD of remote LTM for STFP (FIGS. 14A-14B; Table 1). Importantly, no significant differences between the groups in terms of the total amount of food eaten was found (Table 2). To disentangle effects of the GAF-B construct on the non-social versus social components of the STFP assay, memory tests for odor recognition were examined. Old Pde11a T mice treated with mCherry alone or mCherry-GAF-B spent the same amount of time sniffing the beads (Table 2) and learned equally well during training for non-social odor recognition (NSOR; FIG. 14C). They also demonstrated equally strong recent and remote LTM for NSOR (FIGS. 14D-DE; Table 1). Although not wishing to be bound by any particular theory, this result suggests that disrupting PDE11A4 homodimerization-like genetically deleting PDE11A—does not alter the ability to detect, learn about, or retrieve memories for recognizing non-social odors. In contrast, disrupting PDET 1A4 homodimerization-again, like genetically deleting PD TA-did significantly improve remote LTM for SOR memory (FIG. 14G; Table 1), despite having no effect on SOR learning (FIG. 14F) or total time sniffing (Table 2). Although not wishing to be bound by any particular theory, these data suggest that disrupting PDF11A4 homodimerization is sufficient to reverse ARCD of remote social memory.

TABLE 1
Preference ratios for each group calculated and subequently analyzed by one-sample t-test to
determine if their performance significantly differed from chance (i.e., 0) or determine whether or not a given
group exhibited a significant memory. The resulting P-values within an experiment were then corrected for
multiple comparisons using false discovery rate correction (FDR).

| Datasets | Young | mCh-O | GB-O |
|---|---|---|---|
| FIG. 2A STFP 24 h LTM | t(4) = 9.51, FDR-P < 0.001 | t(4) = 12.60, FDR- | t(14) = 6.74, FDR- |
| | | P < 0.001 | P < 0.001 |
| FIG. 2B STFP 7 d LTM | t(7) = 2.90, FDR-P = 0.036 | t(6) = 0.14, FDR-P = 0.896 | t(5) = 3.84, FDR-P = 0.036 |
| FIG. 2D NSOR 24 h LTM | t(14) = 11.9, FDR- | t(14) = 8.19, FDR- | t(12) = 8.48, P < 0.001 |
| | P < 0.001 | P < 0.001 | |
| FIG. 2E NSOR 7 d LTM | t(14) = 9.75, FDR- | t(14) = 9.45, FDR- | t(14) = 8.04, FDR- |
| | P < 0.001 | P < 0.001 | P < 0.001 |
| FIG. 2G SOR 7 d LTM | t(16) = 7.49, FDR- | t(15) = 2.18, FDR- | t(14) = 6.74, FDR- |
| | P < 0.001 | P = 0.046 | P < 0.001 |

TABLE 2
Amount of food (grams) eaten during STFP tests or amount of time spent invenstigating (seconds)
during SOR/NSOR tests by young controls and old mice treated with mCherry (mCh-O) or GAF-B (GB-O)
lentivirus during 24-hour and 7-day long-term memory (LTM) tests (Data expressed as mean ± SEM).

| Datasets | Young | mCh-O | GB-O | *effect of group: |
|---|---|---|---|---|
| FIG. 2A STFP 24 h LTM | 0.96 ± 0.08 | 1.15 ± 0.11 | 1.23 ± 0.13 | F(2, 15) = 1.63, P = 0.23 |
| FIG. 2B STFP 7 d LTM | 0.94 ± 0.12 | 0.93 ± 0.10 | 0.95 ± 0.05 | F(2, 19) = 0.01, P = 0.99 |
| FIG. 2D NSOR 24 h LTM | 37.06 ± 2.59 | 29.44 ± 2.90 | 29.73 ± 3.44 | F(2, 45) = 2.19, P = 0.124 |
| FIG. 2E NSOR 7 d LTM | 33.47 ± 2.84 | 23.75 ± 2.98 | 26.50 ± 3.13 | F(2, 46) = 2.88, P = 0.067 |
| FIG. 2G SOR 7 d LTM | 52.77 ± 4.48 | 29.38 ± 2.77 | 28.67 ± 2.67 | H(2) = 16.22, P < 0.001 |

*Data analyzed for effect of group by One-Way ANOVA (F) when data passed assumptions of normality and equal variance or by ANOVA on Ranks (H) when assumptions failed. Old mice spent less time sniffing during social odor recognition (SOR) than young mice. There were no effects of group on time spent investigating during non-social odor recognition (NSOR).

PDE11A4 homodimerization is an independent intramolecular mechanism that regulates PDE11A4 trafficking and functioning. As noted above, disrupting PDE11A4 homodimerization significantly changes the subcellular compartmentalization of the enzyme. To better understand the functional consequences of disrupting PDE11A4 homodimerization, we measured PDE activity and cyclic nucleotide levels in cells transfected with GFP+mCherry (i.e., negative control), GFP-PDE11A4+mCherry, or GFP-PDE11A4+mCherry-GAF-B. Compared to the negative control, expression of PDE11A4 significantly increased cGMP- and cAMP-PDE activity in HT-22 cells as expected. This increase in PDE activity was not altered by disruption of PDE11A4 homodimerization (FIGS. 15A-15B), which is consistent with previous studies using purified PDE11A4 enzyme. Also as expected, expression of PDE11A4 in concert with mCherry decreased both cGMP and cAMP levels in COS1 cells relative to the negative control (FIGS. 15C-15D). Interestingly, disrupting homodimerization of PDE11A4 decreased PDE11A4 hydrolysis of cGMP while exacerbating PDE11A4 degradation of cAMP (FIG. 15C-D). The ability of the GAF-B construct to alter cyclic nucleotide levels in absence of a direct effect on PDE11A4 enzymatic activity is likely due to changes in subcellular compartmentalization of PDE11A4 possibly shifting it from a cGMP-rich pool to a cAMP-rich pool. Importantly, disrupting homodimerization not only reduces the accumulation of WT PDE11 A4, it also reduces back to WT levels the potentiated accumulation caused by the aging-related phosphomimic mutant S117D/S124D. Although not wishing to be bound by any particular theory, together these data suggest disruption of PDE11A4 homodimerization alters cyclic nucleotide signaling not by altering PDE11A4 catalytic activity directly, but rather by changing the subcellular localization of PDE11A4 protein (e.g., shifting from a cGMP-rich pool to a cAMP-rich pool).

It was next determined if the isolated GAF-B domain could reduce accumulation not only of WT PDE11A4 but also PDE11A4-S117D/S124D, which mimics the age-related increase in PDE11A4-pS117/pS124 that drives the punctate accumulation of PDE11A4 in the aged brain. Across multiple experiments and cell lines (i.e., HEK293T and HT-22), the isolated GAF-B domain reduced the punctate accumulation of both the WT and PDE11A4-S117D/S124D compared to the mCherry control (FIG. 15E-15F). Together, these data suggest that disrupting PDE11A4 homodimerization may reverse age-related decreases in cGMP signaling by counteracting the effect of age-related increases in PDE11A4-pS117/pS124.

PDE11A homodimerization does not require phosphorylation of S162. As described above, disrupting PDE11A4 homodimerization reduces the accumulation of PDE11A4 in punctate structures both in vivo (FIG. 13F) and in vitro (FIGS. 15E-15F). Since the ability of the isolated GAF-B domain to disperse PDE11A4 accumulations strongly resembles that of a phosphomimic mutation at S162 (i.e., S162D), it was sought to determine if disrupting homodimerization promoted phosphorylation of S162. Such a dispersing phenotype is also triggered by a phosphomimic mutation at S162 (i.e., S162D). As such, it was determined if disrupting PDE11A4 homodimerization may work by promoting phosphorylation of S162. Indeed, S162D changed the subcellular compartmentalization of PDE11A4 in a manner similar to that observed with the isolated GAF-B domain —namely, shifting PDE11A4 from the membrane to the cytosolic fraction (FIG. 15G). The isolated GAF-B domain was able to effectively reduce the accumulation of both WT PDE11A4 as well as the phosphoresistant PDE11A4-S162A, suggesting phosphorylation of S162 is not needed for the dispersing effect of the isolated GAF-B domain (FIG. 15H-151). While not wishing be bound by any particular theory, this result suggests that disrupting homodimerization does not achieve effects by promoting phosphorylation of S162. Indeed, S162D differed substantially from GAF-B in terms of regulating cyclic nucleotide levels. It was found that S162D elicited quite different effects on cyclic nucleotide levels than were described above for the isolated GAF-B domain (FIGS. 15C-15D). Specifically, Si62D did not alter PDE11A4 hydrolysis of cGMP (FIG. 15J) and appeared to reduce PDE11A4 hydrolysis of cAMP (FIG. 15K). Although not wishing to be bound by any particular theory, together these data suggest that homodimerization and pS162 are independent intramolecular mechanism that regulate PDE11A4 trafficking and function.

### Example 4: Pharmacologic Inhibition of PDE11A For Age-related Memory Disorders

Age-related increases in phosphodiesterase 11A (PDE11A), an enzyme that breaks down cAMP/cGMP and regulates social behaviors, may be a fundamental mechanism underlying long-term memory (LTM) deficits associated with age-related cognitive decline. The best controlled studies to date suggest that the longest isoform, PDE11A4, is almost exclusively expressed in the ventral hippocampal formation (a.k.a. anterior HIPP in primates), specifically within neurons of the subiculum, superficial layer of CAT, and the adjacently connected amygdalohippocampal area. This makes PDE11A4 the ONLY PDE to be preferentially expressed in the HIPP, a brain region key to LTMs. Previous studies suggest that cAMP and cGMP signaling are decreased in the aging and demented HIPP (rodents and humans), consistent with the novel observations of aging and dementia-related increases in HIPP PDE11A4 expression in rodents and humans.

This Example describes novel functional studies that support the overarching hypothesis that age-related increases in PDE11A activity drive cognitive decline of LTMs and, thus, it is proposed to develop the first potent, selective and orally-bioavailable small molecule inhibitors of PDE11A4 for the treatment of LTM deficits associated with age-related cognitive decline.

Develop potent, selective small molecule inhibitors of PDE11A using in vitro PDE assays. PDE11A expression increases with aging and dementia in rodents and humans. Viral disruption of PDE11A4 protein in old mice reverses age-related cognitive decline of LTMs, pointing to PDE11A inhibition as a novel therapeutic approach. Screening of a small molecule compound library using a novel yeast-based system identified multiple tractable hits, each with varying degrees of potency and selectivity. Based on existing SAR, these hits are examined to provide CNS-penetrant candidates for evaluation.

Identify improved PDE11A inhibitors using in vitro ADME and screening for functional selectivity in a mammalian cell-based assay. In vitro ADME data on the preliminary screening hits revealed these molecules are likely to be CNS penetrant, with varying other strengths and weaknesses. New analogs are proposed that focus on addressing these weaknesses, while maintaining or improving strengths. A secondary cell-based assay validates candidates in a mammalian context and assess the potential for functional selectivity (e.g., the ability to preferentially inhibit cGMP-hydrolytic activity vs. cAMP-hydrolytic activity).

Assess behavioral and physiological efficacy, and side effect liability in vivo. Acute disruption of PDE11A4 in the hippocampus of old mice is able to reverse age-related LTM deficits, and genetic deletion of PDE11A reduces age-related decline of LTMs. Although not wishing to be bound by any particular theory, these findings suggest that inhibition of PDE11A4 not only prevents, but may also reverse age-related LTM deficits. Here, aged C₅₇BL6 mice from the NIA aging rodent colony and aged Pdella mutant mice as “disease” models are used for in vivo screening. PK/PD studies are used to establish target engagement (i.e., brain exposure), target modulation (i.e., increased phosphorylation of known PDE11A4 downstream target pS6-235/236), as well as pretreatment interval (tmax) and specific doses (comparing brain exposures vs. mammalian cell-based assay ECSo). Behavioral studies assess both recognition and associative LTMs in efficacy screens, with attention paid to potential sensory/motor confounds. So that modulation of a relevant physiological endpoint is assessed, neuronal activity and functional connectivity underlying memory retrieval are assessed by measuring expression of the activity-regulated gene Arc. Finally, side effect liability is assessed in studies examining general health parameters, locomotor activity/coordination, behaviors related to anxiety and depression, and social interactions. Importantly, the lead compound is profiled in vivo using young vs. old PDE11A mutant mice, to verify compound efficacy replicates in a second cohort of mice (=see effects in wild-types) and is mediated by inhibition of PDE11A (=no effect in knockouts).

After the age of 60, nearly all individuals experience some form of cognitive decline—particularly memory deficits—and no drugs are able to prevent or reverse this loss. Indeed, advanced age is the strongest risk factor for dementia. Even in absence of dementia, age-related cognitive impairment increases health care costs and risk for disability. Age-related cognitive decline is not a uniform process, with variability in symptom severity observed across individuals and cognitive domains. For example, associative memories are more susceptible to age-related cognitive decline than are recognition memories for individual items for reasons that are not clear but may be related to deficient activation of anterior hippocampus. This lack of knowledge slows therapeutic development.

Consistent with PDE11 A4's restricted expression pattern, Pde11a KO mice appear normal on a wide range of sensory, motor and anxiety/depression-related behaviors, show no gross peripheral pathology at least up to 1 year of age (later ages not assessed), and reproduce normally. Instead, young adult Pde11a KO mice exhibit select social phenotypes such as preferring to interact with other Pde11a KO mice vs wild-type (WT) mice and showing differences in the consolidation of social memories. Pde11a KO mice also have an increased sensitivity to the behavioral effects of lithium. Little is known of the signaling pathways lying up or downstream of PDE11A4, but it was shown that PDE11A appears to regulate important signals for memory consolidation, including glutamatergic and calcium/calmodulin-dependent kinase II (CamKII) signaling, as well as protein synthesis.

Although not wishing to be bound by any particular theory, these results suggest that cAMP and cGMP signaling is decreased in the aged and demented hippocampus (rodents and humans), particularly when there is a history of traumatic brain injury (TBI). These age-related decreases in cyclic nucleotides are consistent with the observations that PDE11A4 expression increases with age in the rodent and human hippocampus and is significantly elevated in hippocampus of demented vs. non-demented aged humans with a history of TBI (FIGS. 14A-14C). Hits were identified in a screen for PDE11A inhibitors representing the only known molecules to demonstrate that pharmacological selectivity is achieved versus other PDEs. Studies are performed to identify potent, selective and orally-available PDE11A inhibitors with drug-like properties. It is hypothesized that pharmacological inhibition of age-related increases in HIPP PDE11A4 is a mechanism to rescue age-related cognitive impairment of social aLTMs.

Human studies show that associative memories involving verbal and/or non-verbal stimuli (e.g., faces and names) are more susceptible to age-related cognitive decline than are recognition memories for individual items. A differential sensitivity of aLTMs vs recognition LTMs (rLTMs) was recapitulated in mice using social memory tasks (FIGS. 15A-15C). The fact that SOR/NSOR remains intact suggests STFP deficits do not reflect a global olfactory deficit. As used in this example, memory refers to eating more of the trained food vs. novel food (FIG. 15A) or investigating the novel odor longer than the familiar odor (FIGS. 15B-15C). Although not wishing to be bound by any particular theory, these findings, in combination with the human studies noted above and a report showing rapid decay of social aLTMs in aged rats, suggest this enhanced vulnerability of social aLTMs to age-related cognitive decline is conserved across species.

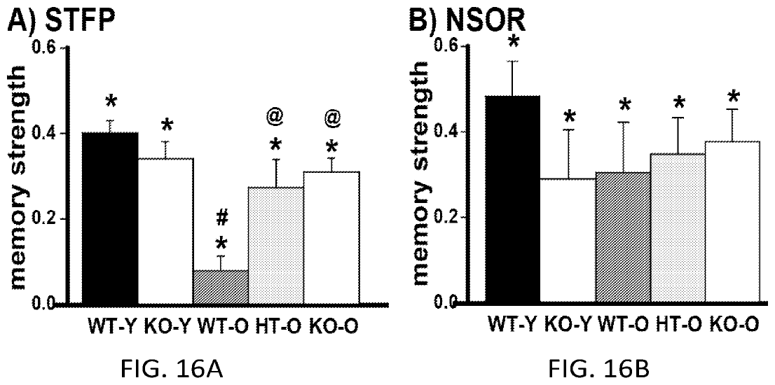

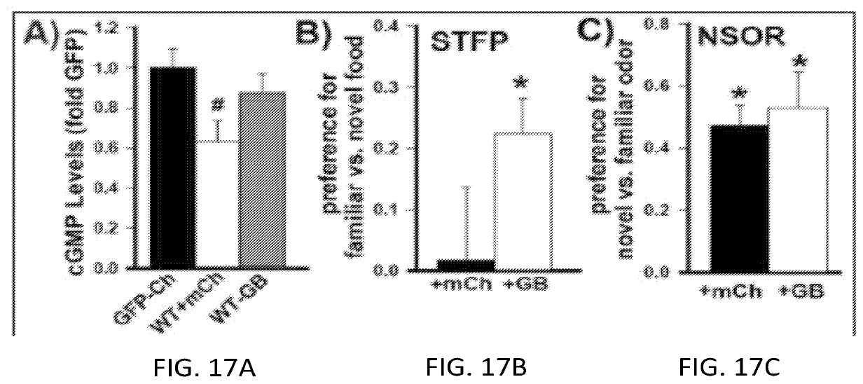

To test the hypothesis that age-related cognitive decline of aLTMs is driven by the age-related increases in PDE11 A4 expression described above (FIG. 14A), young vs. old Pdella WT and KO mice in social transmission of food preference was compared (STFP; FIGS. 16A-16B). The protective effect of PDE11A deletion was replicated across the sexes in 2 large cohorts, the combined analysis of which is shown in FIGS. 16A-16B (n=26-29/group). The protective effect of PDE11A deletion on aLTMs is specific because there is no difference in rLTM between WT-O and KO-0 mice, as measured in non-social odor recognition (NSOR; n=14). The initial focus on social aLTM stems from the fact that in young adult mice, PDE11A4 regulates social behaviors and social memory, but not non-social memory. While aging severely impairs PDE11A WT mice, old KO mice show robust aLTM for STFP on par with that of young PDE11A WT mice. These findings were replicated in two large cohorts of male and female mice (no effect of sex; combined data shown in FIGS. 16A and 16B), underscoring the reproducibility of the protective effect. Further, this protective phenotype was blocked by virally overexpressing PDE11A4 in the hippocampus of Pdella KO mice (i.e., mimicking the state of an old WT). Perhaps even more importantly, age-related STFP memory deficits in old WT mice were reversed using a peptide that disrupts PDE11A4 homodimerization and, thus, its cGMP-hydrolytic activity (FIGS. 17A-17C). Together, these data show that 1) age-related cognitive decline is reversible and 2) PDE11A4 inhibition is a means of achieving such rescue.

Pyridopyrazole 3001 was evaluated for in vitro pharmaceutical properties and the results are shown in Table 1 below

TABLE 1
in vitro ADME properties of PDE11A Compound 3001

| | | Mouse Liver | | Human Liver | | | | | |
|---|---|---|---|---|---|---|---|---|---|
| | | Microsome | | Microsome | | Human | Human | Human | |
| | Solubility | Stability t½ | | Stability t½ | | CYP3A4 | CYP2D6 | CYP2C9 | |
| | in PBS¹ | (min)/Clint | | (min)/Clint | | IC50 | IC50 | IC50 | MDCK1 |
| Cpd | (μM; pH 7.4) | μ/min/mg | | μl/min/mg | | (nM) | (nM) | (nM) | A:B |
| 3001 | 54 | <2 | >690 | <2 | >690 | 3000 | >10000 | 4900 | 0.78 |

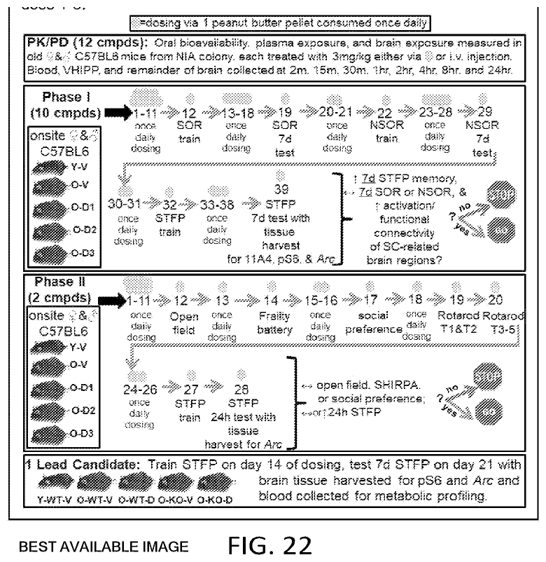

Given PDE11A4's uniquely restricted expression pattern, this example aims to not only uncover the neurobiological role of the enzyme, it may also define the function of an anatomically restricted, molecularly defined neuronal population that may be specialized for processing social information and exceptionally vulnerable to age-related cognitive decline. The focus on LTMs for social experiences is relatively unique in the field of learning memory. Most studies tend to utilize fear conditioning, novel object recognition or various spatial learning paradigms (e.g., water maze). Further, what studies have examined the molecular/anatomical mechanisms of social memory have largely focused on short-term memory (STM). Here, the effect PDE11A inhibitors on recent LTMs (24 h post training) and remote LTMs (7 days post training; FIG. 22) is compared/contrasted.

### Synthesis and PDE Inhibition

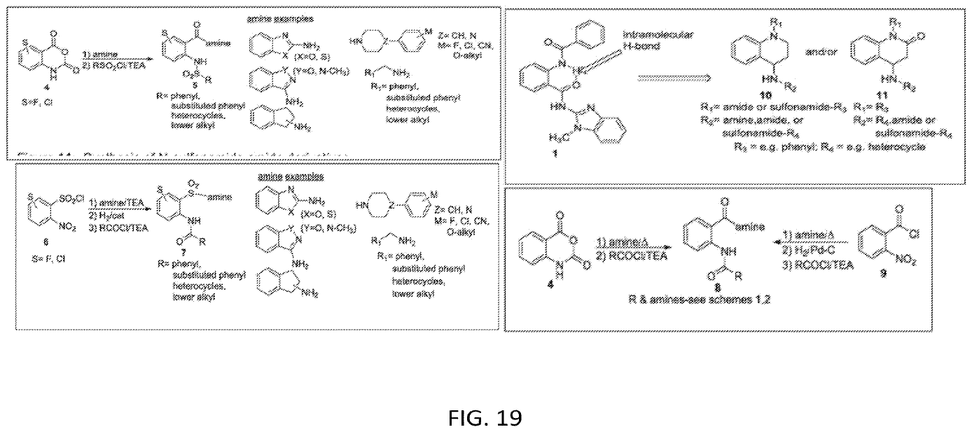

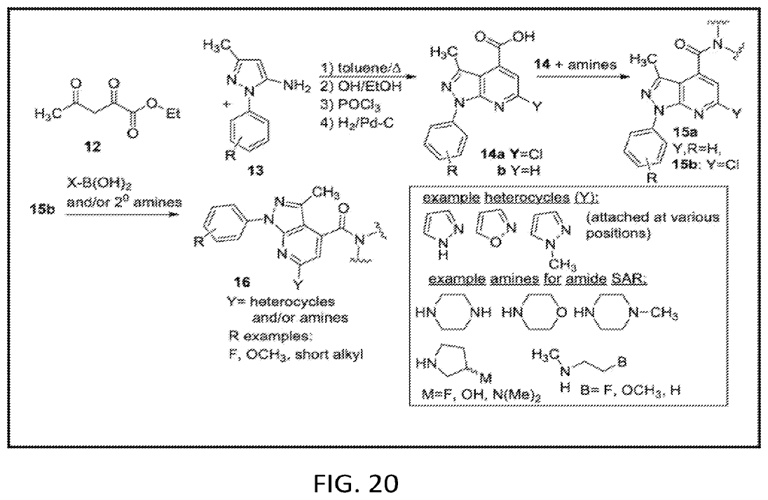

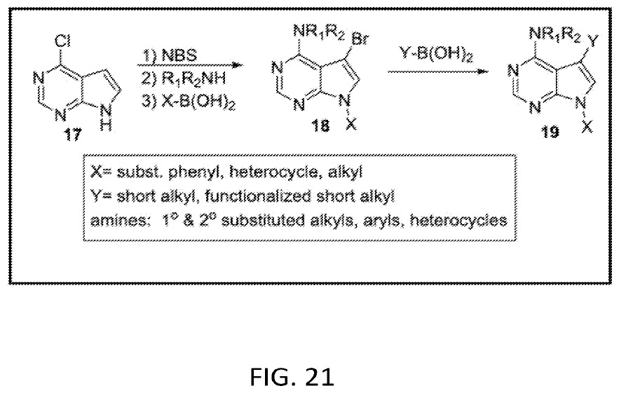

Non-limiting examples of structures and syntheses of compounds of the disclosure are shown in FIGS. 19-21.

In vitro enzyme assays are conducted via the Ba(OH)₂ precipitation method of Wang et al. using commercially-available recombinant PDEs representing all 11 PDE families. Assays for PDE11A inhibition are carried out with both 200 nM cAMP and 100 nM cGMP. Substrate concentrations for the remaining PDEs are 100 nM cGMP (PDE1C), 1 μM cGMP (PDE2A), 30 nM cGMP (PDE3B), 120 nM cAMP (PDE4D), 500 nM cGMP (PDE5A), 1.7 M cGMP (PDE6C), 15 nM cAMP (PDE7A), 10 nM cAMP (PDE8A), 70 nM cGMP (PDE9A), and 30 nM cAMP (PDE10A). PDE11A assays are carried out in an 11-point 1:2 titration from 50 μM to ~1 nM to determine IC₅₀ values from at least three independent experiments. Compounds displaying IC₅₀ values of <50 nM against PDE11A are tested at 1p M against the remaining 10 PDEs to assure at least a 20-fold level of selectivity. Those compounds for which the IC₅₀ against PDE11 A is <50 nM and demonstrate at least 20-fold selectivity versus other PDEs advance to ADME property evaluation

### In Vitro ADME Evaluation and Mammalian Cell Based Assay

In vitro ADME properties of suitable PDE11A inhibitors are assessed and compounds meeting criteria move onto a secondary mammalian cell-based assay. This secondary screen confirms compounds are taken up by mammalian cells and inhibit mouse PDE11 A (species used for in vivo screen). Suitable compounds advance to in vivo pharmacokinetic evaluation.

Metabolic stability: Samples at 37° C. in the presence of human or mouse liver microsomes and NADPH according to standard methods. Aliquots are removed at 5 time points, quenched and analyzed (LCMS/MS and MS/MS as needed) for remaining test compound, along with a positive control. Microsomal protein content is adjusted to give consistent results. Data are reported as half-life and clearance. Assay acceptance criteria is 20% for all standards and 25% for the LLOQ.

Aqueous solubility: Kinetic aqueous solubility is measured by adding ~2 mg samples to pH 7.4 buffered aqueous solutions at 25° C. The mixture is agitated at 25° C. for 1 hour, filtered and evaluated by UV and/or LCMSMS analysis. Data are reported as mg/mL and μM concentration.

CYP inhibition: Compounds are assessed for their ability to inhibit the three major human cytochrome P450 enzymes, 3A4, 2D6 and 2C₉. Expressed enzymes are used to minimize non-specific binding and membrane partitioning issues. The 3A4 assay uses testosterone as a substrate and is analyzed by LC/MS/MS on a Waters TQ instrument using positive or negative electrospray ionization. The 2D6 and 2C₉ assays use fluorescent substrates and are analyzed on an Envision Plate Reader.

MDCK permeability: MDCK cell monolayers (Absorption Systems, Malvern, PA) are grown to confluence on collagen-coated microporous membranes in 12-well assay plates. Assay buffer consists of Hanks' balanced salt solution containing 10 mM HEPES and 15 mM glucose at pH—7.4. The buffer in the receiver chamber also contains 1% bovine serum albumin. Compounds are tested at a final concentration of 5 μM in the assay buffer. Cell monolayers are dosed on the apical side (A-B) or basolateral side (B-A) and incubated at 37° C. with 5% CO₂ in a humidified incubator. Samples are taken from the donor and receiver chambers at 120 minutes. Each determination is performed in duplicate. The flux of Lucifer yellow is also measured post-experimentally for each monolayer to ensure no damage is inflicted to the monolayer during flux period. Samples are assayed on a Waters TQ LC/MS/MS using positive or negative electrospray ionization.

hERG inhibition: HEK293 cells stably transfected with the hERG ion channel are grown to 80% confluency and then seeded into poly-lysine coated plates (25,000 cells/well). Cells are loaded with Thallos dye, treated with test compounds at 1 and 10 μM (final DMSO concentration=0.10%) and then thalium flux is measured on a plate reader (excitation @480 nM; emission @530 nM) according to published procedures. Data are reported as percent inhibition at both concentrations.

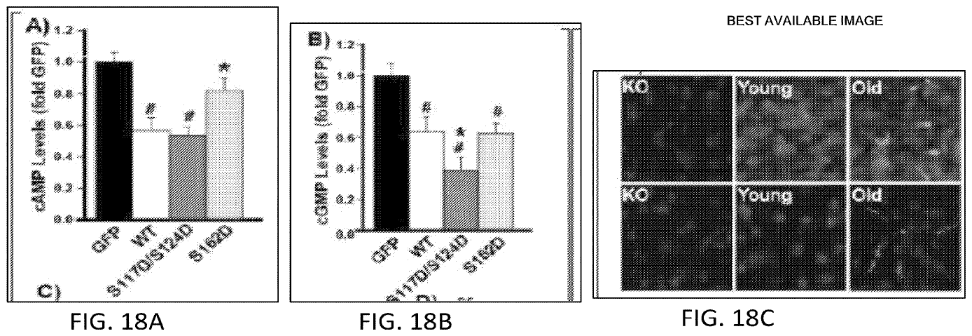

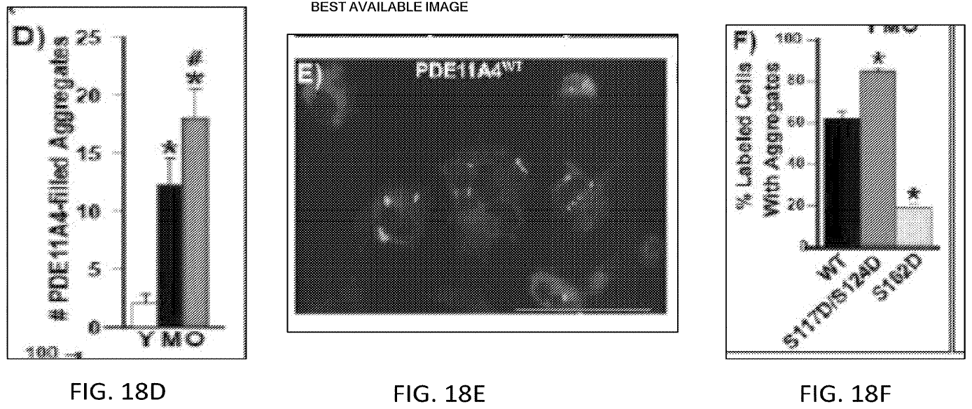

Mammalian cell-based functional assay: COS1 monkey fibroblast cells transiently transfected with mouse PDE11A (95% homologous to human PDE11A4) are used to confirm inhibition in a mammalian context and assess effects on subcellular compartmentalization (FIGS. 18A-18F). Cells transfected with the negative control green fluorescent protein (GFP) are treated with vehicle and cells transfected with GFP-tagged mPDE11A are treated with either vehicle or compound (3 concentrations selected based on the 5-FOA EC₅₀ and in vitro IC₅₀, n=6/group). Cells are imaged to quantify changes in mPDE11A subcellular compartmentalization (FIGS. 18A-18F) and harvested to measure both cAMP and cGMP (as previously reported) to assess potential functional selectivity of enzymatic inhibition (e.g., preferential inhibition of cGMP vs. cAMP hydrolytic activity)

### In Vivo Compound Evaluation:

The selection of C₅₇BL6 as the mouse strain of choice is driven by the fact that 1) mice from the NIA aging rodent colony are used for as many studies as possible in order to limit husbandry costs, 2) only C₅₇BL6 and Balb/cBy mice are available through the NIA aging rodent colony, and 3) the C₅₇BL6 strain is the closer of the two to the genetic background of the Pdella mutant mouse line (87% C₅₇BL/6J/12.4% C₅₇BL6/N/1%129S6,). Because NIA limits orders to only 20 mice per month, a breeding colony of C₅₇BL6 mice is established at USC using founders from the NIA stock (i.e., as opposed to the C₅₇BL/J mice on hand). The fact that cohorts of delivered mice have a differential stress history relative to those bred onsite is factored in when interpreting data, and delivered mice are given an extended 2 week habituation to the facility prior to experimentation to mitigate stress effects related to shipment. Mice delivered from NIA are used in PK/PD studies; whereas, those bred onsite are used in behavioral analyses. For studies throughout this Example, “Young” refers to 2-5 months of age and “Old” refers to 18-22 months of age since 1) the protective effects are seen in PDE11A KO mice as early as 14 months old and 2) it is expected 50% of the colony to die by the age of 24 months (i.e., mice with exceptional longevity are not studied).

Pretreatment interval and the specific doses tested for each compound are empirically determined. The pretreatment interval is dictated by the tmax established in the in vivo PK study. The logarithmic dose range for behavioral testing is based on the active moiety and selected based on brain exposures from the PK study in comparison to the mammalian cell-based assay EC₅₀. In vivo dose ranges for compounds with a nanomolar in vitro EC₅₀ typically fall within the range of 0.1-3 mg/kg; whereas, in vivo dose ranges for compounds with a micromolar in vitro EC₅₀ typically range from 1-30 mg/kg. Given the high cost of animal husbandry, the 20 mouse/month order limit set by NIA, and the need for a minimum of n=10/sex/group (=100 mice/behavioral experiment), behaviorally profile compounds following both acute and chronic dosing is not performed. Compounds are not tested with naive cohorts for each behavioral assay tested. Thus, chronic dosing is utilized over acute dosing because a chronic dosing schedule better enables repeated behavioral testing as it is likely to yield steady-state levels of compound and avoid sensitization associated with a dose-withdrawal-dose schedule. A chronic dosing regime is used because 1) chronic dosing better mirrors the time course of PDE11A lentivirus (FIG. 16) mice habituates over time to any stress that may be associated with dosing, 3) having drug on board during all phases of a memory assay (i.e., acquisition, consolidation and retrieval) mitigates concerns about potential state-dependent effects on memory performance drugs are typically dosed chronically in a clinical setting. Compounds are delivered orally via peanut butter pellets. This method ensures that each mouse receives a fixed dose of compound within a relatively short amount of time (because of high palatability) and avoids many of the problems that is associated with oral delivery via chow pellets (e.g., typically consumed over long periods of time), water (e.g., solubility issues), or oral gavage (e.g., risk of stress, injury, and death; for extensive review, see Introduction of).

In vivo screening trees evaluate 1) target engagement, 2) target modulation, 3) efficacy in the face of a relevant perturbation, 4) modulation of a physiological endpoint, and 5) side effect liability. As such, in vivo characterization begins with pharmacokinetic/dynamic studies (PK/PD). The integrity of the blood brain barrier is known to deteriorate with age; thus, old male and female C₅₇BL6 mice from the NIA aging rodent colony are used in these studies. Studies are designed in such a way that the same cohort of mice is used to establish oral bioavailability (% F; i.e., oral vs. i.v. [plasma]), target engagement (i.e., drug exposure in the brain), and target modulation (i.e., increased phosphorylation of established downstream target pS6-235/236 in the VHIPP. Mice are fasted and then administered a 3 mg/kg dose either orally via peanut butter pellet or by i.v. injection. Trunk blood, the VHIPP, and the remainder of the brain is then collected at 2 min, 15 min, 30 min, 1 hr, 2 hr, 4 hr, 8 hr and 24 hrs (oral, n=2/sex/time; i.v. n=1-2/sex/time). Upon collection, blood samples are placed into chilled, EDTA-fortified tubes, centrifuged for 10 minutes at 3000 rpm (4° C.), and resulting plasma aliquoted into 96-well plates for Liquid Chromatography-Tandem Mass Spectrometry (LC/MS/MS). The VHIPP (for processing via Western blot to measure pS6) and the brain samples leftover following dissection of the VHIPP (for processing by LC/MS/MS to measure drug exposure) are rinsed in PBS, snap frozen and separately stored at −80° C.

According to standard practices, screening trees move from least to most stressful/invasive, with tests separated by at least 1 day (FIG. 22). To assess efficacy in the face of a relevant perturbation, compounds in old mice, as opposed to healthy young mice are tested. Phase I and II are conducted in C₅₇BL6 male and female mice bred onsite and includes a total of 5 treatment groups: vehicle-treated young mice, vehicle-treated old mice, and 3 groups of old mice each treated with 1 of 3 different doses of compound (n=10/sex/group; FIG. 22). There are no clinical standards for reversing age-related cognitive decline; thus, a pharmacological “positive control” group is not included. That said, inclusion of a young vehicle-treated group ensures the assay itself is performing as expected on the test day (e.g., that it was possible to read out strong memory in the STFP assay). Phase I focuses on compound efficacy in the 7d STFP assay; however, to interpret effects in STFP SOR and NSOR are also assessed since an effect on STFP could manifest as a result of stronger stimulus detection or encoding as opposed to a true enhancement of the association between the 2 stimuli. Since STFP requires food restriction, SOR is tested first, then NSOR, then STFP, as has been done for the genetic/viral cohorts (FIG. 22). Previously, it was shown that stronger 7d STFP memory retrieval caused by PDE11A deletion correlates with stronger neuronal activation and changes in functional connectivity of brain regions related to the consolidation of remote aLTMs. Therefore, to test for modulation of a relevant physiological endpoint, mice are killed immediately at the end of their 7d STFP test and brains harvested and hemisected so that changes in neuronal activity/functional connectivity are assessed via expression of the activity-regulated immediate early gene Arc in one hemisphere as well as protein expression of PDE11A4, other closely related PDEs (e.g., PDE2, PDE10) and pS6-235/236 using the VHIPP of the other hemisphere. It is determined if PDE11i's downregulate PDE11A4 protein since PDE11A HTs express 50% of mRNA, but only 20% of protein, suggesting PDE11A is self-regulating. Of note, genetic deletion of PDE11A does not appear to trigger compensatory upregulation of closely related PDEs; therefore, compensation of other PDEs is not be expected in response to PDE11i's.

The 2 compounds best able to improve remote 7d STFP aLTM and strengthen neuronal activity/connectivity in brain regions related to the storage of remote aLTMs —without changing 7d SOR or NSOR rLTM—are counterscreened for side effect liability in Phase II (FIG. 26; general side-effects: general health parameters, locomotor activity/coordination; PDE11A-specific side effects: recent STFP aLTM and social preference-see more below). Locomotor activity is assessed first using the open field as per the previous methods so that potential sedatory/stimulatory effects (e.g., changes in total distance travelled or movement speed) are detected as well as potential anxiolytic/anxiogenic-like effects (i.e., changes in distance travelled/time spent in center:periphery). General health (e.g., body weight and temperature, reflexes, muscle and skin tone, etc) is then assessed using the “Frailty” battery, which is highly similar to the SHIRPA observation screen used previously in adult Pde11a KO mice and saw no effects. These same mice are then tested to determine if the PDE111 alters social preference. Previously, the “mouse psychiatry” assay was described where male and female C₅₇BL/6J or a Pde11a KO mice were given an opportunity to explore a 3 compartment chamber with a perforated plexiglass cylinder at either end that contained either a novel sex-matched Pde11a WT or novel Pde11a KO. After 25 minutes of interaction, it was found that male and female C₅₇BL/6J mice preferred to interact with Pde11a WT vs. KO mice; whereas, Pde11a KO mice preferred to interact with KOs vs. WTs. Importantly, C₅₇BL/6J mice did not show a consistent preference when interacting with a Pde11a WT vs. HT, suggesting a PDE11i do not alter social behaviors that impact social preference. Here, the young vehicle-treated C₅₇BL6 mice act as the ‘mouse psychiatrist’ and be given an opportunity to interact with a vehicle-treated old C₅₇BL6 vs. a drug-treated old C₅₇BL6. Next, mice are tested in a 2-day rotarod protocol, as has been previously published. This protocol allows for assessment of both motor coordination on day 1 as well as the phenomenon of “spontaneous improvement” that occurs during the consolidation of motor memory between days 1 and 2. Deficits in spontaneous improvement have been noted in patients with depression and animal models thereof.

Recent STFP aLTM as well as Arc expression as a surrogate marker of neural activity/connectivity is assessed. Although young to middle aged Pde11a KO mice show intact STM and intact or improved remote LTM, they show no recent LTM for social experiences. Young PDE11A HT mice-which more realistically reflect the loss of PDE11A activity that is achieved with an inhibitor-do exhibit recent LTM for social experiences and old PDE11A HTs are still protected against age-related cognitive decline of aLTMs for STFP (FIG. 22). Thus, transient amnesia is not required for PDE11A knockdown to strengthen remote aLTMs. It is hypothesized that Pde11a KO mice exhibit transient amnesia by virtue of having expedited systems consolidation. SC is the process by which a hippocampal memory is relocated to the cortex and subsequently erased/silenced from the hippocampus. As the memory trace undergoes systems consolidation, the origin and destination of retrieval vectors must also be updated. If PDE11A deletion relocates and erases the hippocampus memory ahead of schedule without updating the retrieval vector, this effectively creates a “blackout” period by temporarily misplacing the memory.

In the final phase of testing, active candidate(s) are profiled using the Pdella mutant mouse line to establish effects of the compound is due to inhibition of PDE11A. Specifically, vehicle-treated young wild-type mice, vehicle- and drug-treated old wild-type mice, as well as vehicle- and drug-treated old knockout mice are tested for remote STFP aLTM and neuronal activity/connectivity (FIG. 22). Trunk blood is also collected for metabolomic profiling using the Biorad Bioplex platform.

### Results

Based on the fact that PDE11A KO mice are protected against age-related decline of aLTMs, and disrupting PDE11A4 function using a peptide is capable of reversing age-related decline of aLTMs in old WT mice, it is anticipated that pharmacological inhibition of PDE11A4 similarly is able to reverse age-related cognitive decline. This interpretation is based on two primary results. First, PDE11A inhibitors improve memory in STFP in PDE11A WT mice. Second, PDE11A inhibitors have no effect in PDE11A KO mice. Further, it is expected PDE11i's to produce favorable side effect profiles since 1) expression of the enzyme is relatively restricted and Pde11a KO mice appear normal on a wide range of sensory, motor and anxiety/depression-related behaviors, show no gross changes in general health or peripheral pathology, and reproduce normally. Such a profile is interpreted as proof in favor of developing and pursuing PDE11A4 inhibitors as potential therapeutics for age-related decline of aLTMs.

In a non-limiting example, alternative methods and starting materials for synthesis of target compounds are utilized. In another non-limiting example, a bolus administration once/day may not provide a window of PDE11A inhibition that is sufficiently long to impact memory consolidation, given consolidation requires multiple waves of intracellular signaling across the first 24 hours if not for several days. In another non-limiting example, if none of the first 3 PDE11i's produce behavioral effects similar to those has been observed using genetic approaches, the drug is delivered 2-3 times per day (depending on ½ lives) or via implanted Alzet osmotic mini-pumps that enable continuous drug delivery. In another non-limiting example, two additional groups of mice are included—1 group treated with the GAF-B lentivirus that reverses age-related STFP deficits (see FIG. 22) and a 2^{nd} group with the respective negative viral control. Although including these additional groups reduce the total number of compounds screened in vivo, the GAF-B infused group could then serve as a “positive control”, albeit a genetic one.

### Statistical Analyses:

Proposed n's (in vivo: n=10/sex/group) were based on a priori power analyses of assay-specific historical minimum detectable differences in means and standard deviations (α=0.05, 1−β=0.8). Power is also confirmed post hoc. In Western blot experiments, samples generally span multiple blots. To account for non-specific technical differences across blots (e.g., transfer or antibody binding efficiency, etc.), all biochemical data are normalized as a fold change of the control group on a given blot. All datasets meeting normality and equal variance assumptions are tested by parametric statistics; those not meeting assumptions are tested by nonparametric statistics. In general, data are analyzed by multifactorial ANOVAs or by repeated measure ANOVAs where appropriate to account for multiple comparisons. For example, in vivo data are analyzed for effects of sex, genotype, and drug treatment in addition to assay-specific factors, such as food type. Statistical outliers (>2 standard deviations from the mean) are dropped from analyses. Significant ANOVAs are followed by Student Newman-Keuls (following parametric analyses) or Dunn's post hoc tests (following non-parametric), with P<0.05 defining significance. Data plotted means ±SEMs.

Data rigor:

The rationale for the disclosure is based on both mouse and human data, which strengthens rigor. Further, the primary behavioral findings upon which this disclosure is based have been replicated across multiple cohorts of mice.

Genetically modified mice are genotyped a priori to enable proper counterbalancing of experimental run lists (and genotypes reconfirmed post death by Western blot or in situ hybridization); however, experimenters are blind to genotype or drug treatment at the time of data collection/analysis. To accomplish the latter, blinded drug pellets are prepared by an experimenter different than the one conducting the behavioral experiments. As done with the genetic studies, each cage of old mice include 1 subject from each treatment group to counterbalance any slight environmental differences that may arise between cages and to avoid litter effects (since most mice within cage tend to be from the same litter). Physical parameters are counterbalanced across subjects (e.g., which is the “trained” vs. “novel” spice).

Biological Variables: Equal numbers of males and females are always tested to test for effect of sex.

### SUMMARY

These innovative studies provide much needed pharmacological tools for probing PDE11A function along with insight into the fundamental mechanisms of age-related cognitive decline. Although the PDE11A inhibitor identified previously is highly selective and even commercially available, it is a poor tool compound in that it is highly insoluble and does not appear to cross the blood brain barrier or demonstrate target engagement in vivo. Thus, if successful, this research advances the PDE11A field by providing further support of PDE11A4 as a possible therapeutic target and developing the lead candidates for pursuit thereof. PDE11A is a member of a highly druggable enzyme family that is positioned to selectively control cyclic nucleotide signaling in a molecularly-defined population of neurons, without affecting signaling elsewhere. This can diminish age-related deficits in at least social aLTMs without causing unwanted side effects. The fact that PDE11A is a realistic candidate for drug development increases the value of developing small molecule inhibitors and understanding its function. In a non-limiting example, the characterization of lead candidates are broadened. Effects on social conditioned place preference, a different type of social aLTM, are examined as effects on nonsocial types of aLTMs. It is also of interest to test the lead PDE11 Ai's in other cognitive domains including spatial memory, other depression-related behaviors, additional anxiety-related behaviors, and sensorimotor gating. Although PDE11A deletion in young adults appears to preferentially affect social behaviors at baseline, these compounds are characterized beyond the social domain because age-related increases in PDE11A expression may be ectopic in. It is also of interest to determine if PDE11i's, like genetic deletion of PDE11A, can modulate lithium responsivity. In a non-limiting example, the mechanism by which a PDE11i may improve age-related cognitive decline and determine therapeutic windows for lead candidates (i.e., establish maximum tolerated dose, tissue toxicity, etc.) is examined.

### Example 5: PDE11A Inhibitors for CNS Disorders

To test the hypothesis that age-related cognitive decline of aLTMs is driven by the age-related increases in PDE11A4 expression, young vs. old Pde11a WT and KO mice were compared in social transmission of food preference (STFP; FIGS. 16A-16B). FIG. 16A shows aged male and female PDE11A WT mice show no STFP memory 7 days after training. Aged PDE11A KO mice show robust memory equivalent to young (Y) mice. This protective effect was replicated across both males and females in two large cohorts (26-29/group). The protective effect of PDE11A is specific for memory as evidenced by FIG. 16B that illustrates that there is no difference in recognition long term memory (rLTM) among WT and PDE11A KO young mice, as measured by non-social order recognition (NSOR). FIGS. 15A-15C show a comparison of young and old mice that display reduced aLTMs based on STFP, and no reduction in rLTMs based on NSOR, or social order recognition assays.

### Chemistry

These compounds were profiled further using additional commercially available analogs and by in vitro ADME characteristics. The synthesis of these analogs is illustrated in the schemes of FIGS. 24-26. FIG. 27A shows an exemplary group of compounds of the disclosure. In vitro PDE11A inhibition of compounds of the disclosure is shown in FIG. 27B. FIG. 27B provides data demonstrating the comparative potency of the listed compounds as PDE11A4 inhibitors. In the rightmost column of FIG. 27B, percent (%) values refer to percent inhibition, and non-percent values refer to IC₅₀ values. Compounds were screened at 50 nM and 500 nM concentrations, and based on the degree of inhibition shown relative to a control compound, the IC₅₀ values for selected compounds were then determined, and are shown in FIG. 27B as non-percent values.

### Synthesis

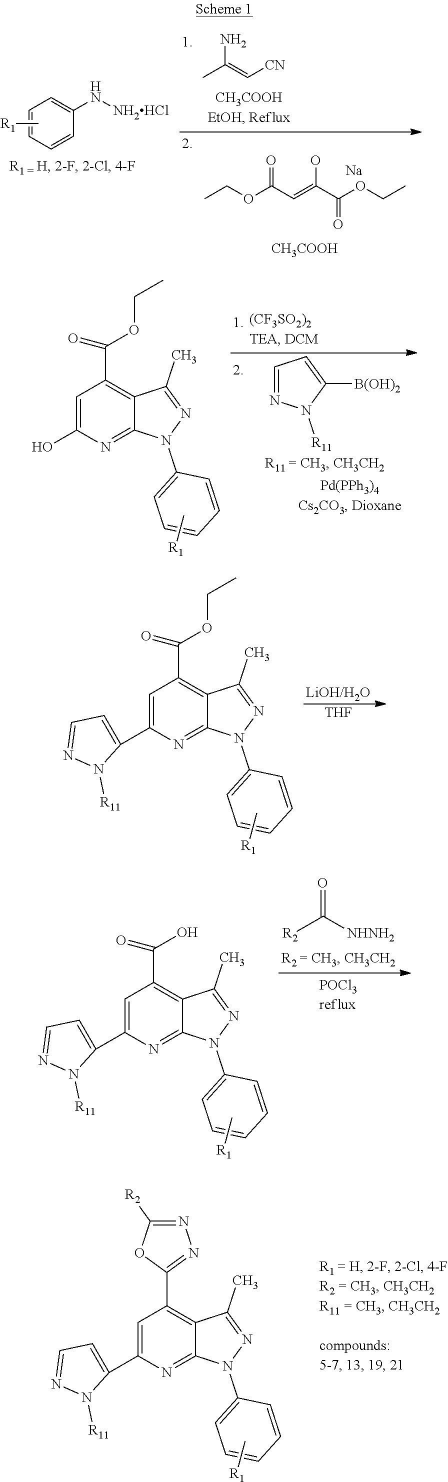

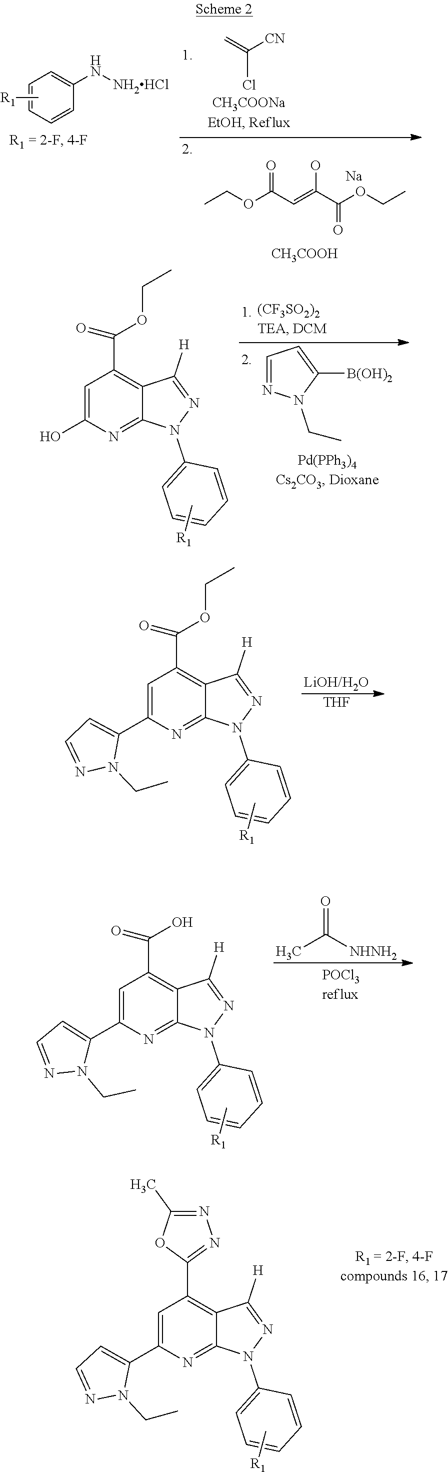

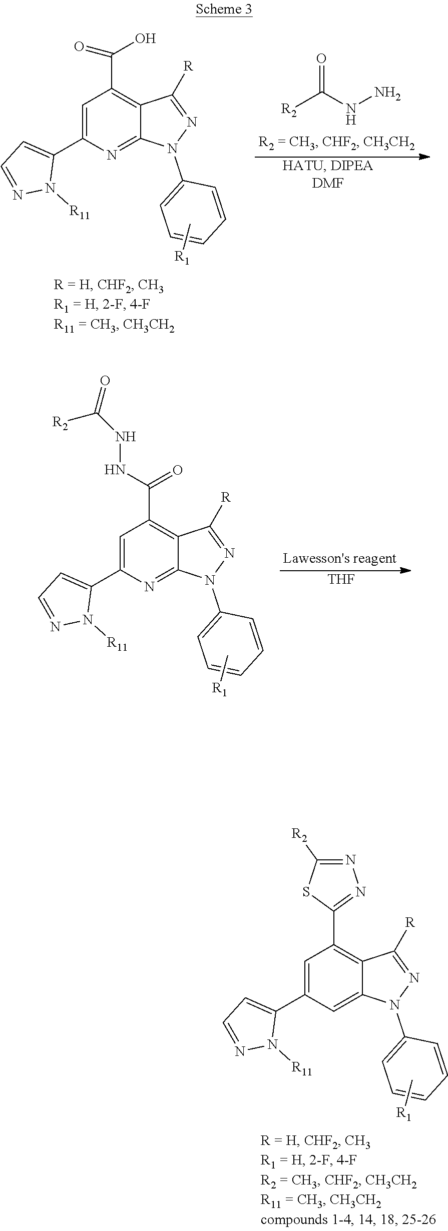

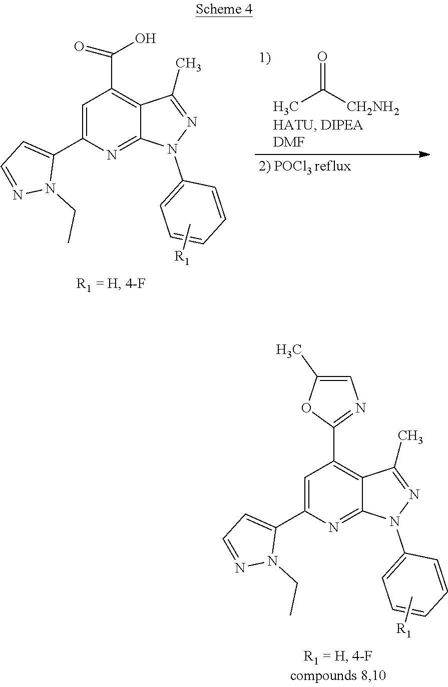

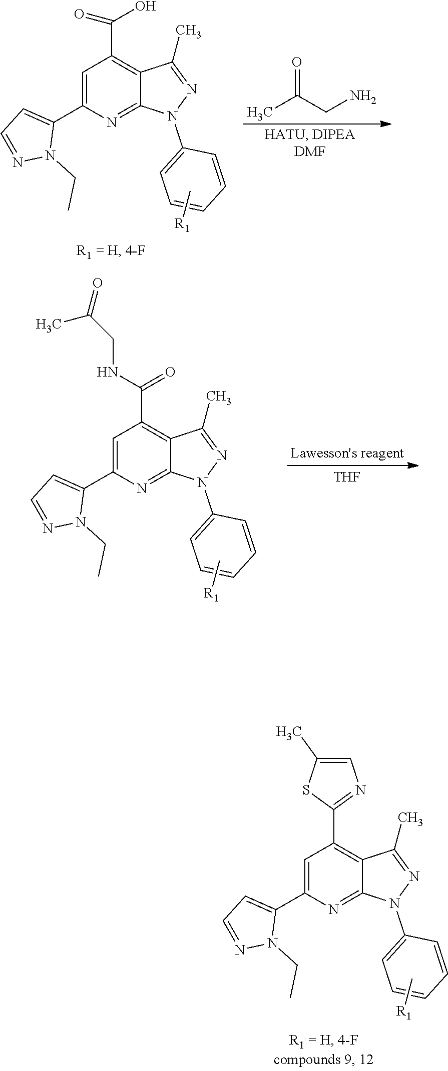

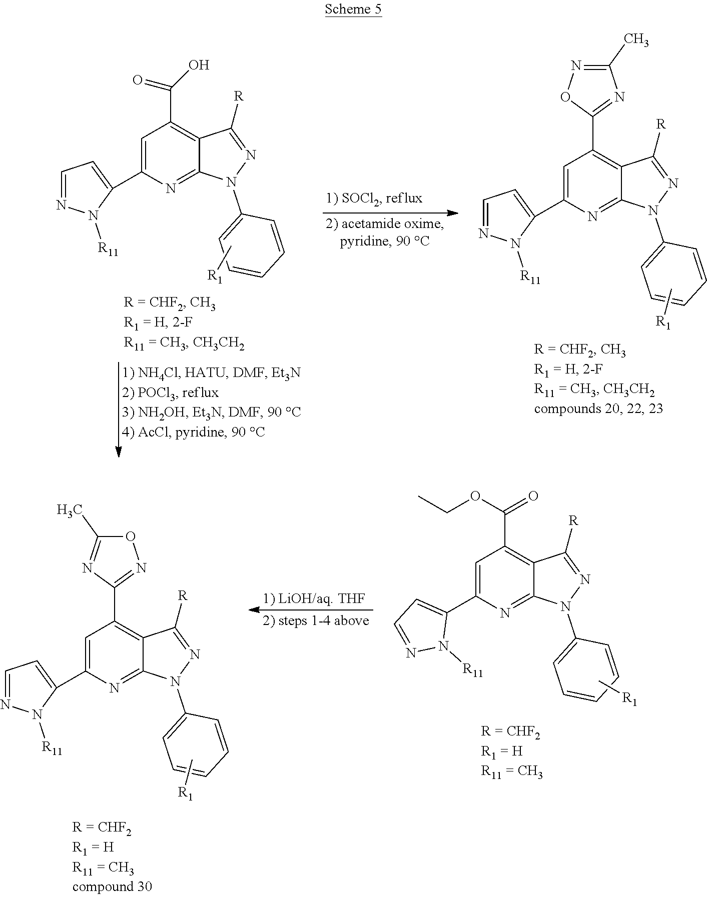

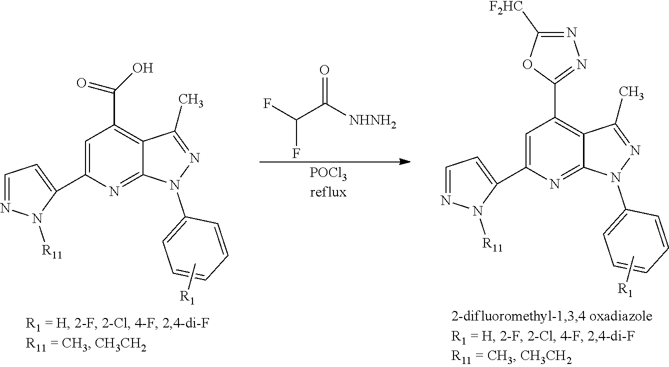

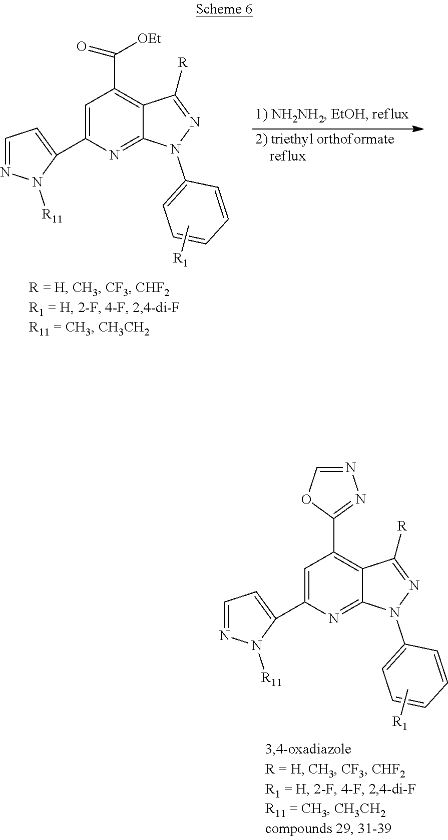

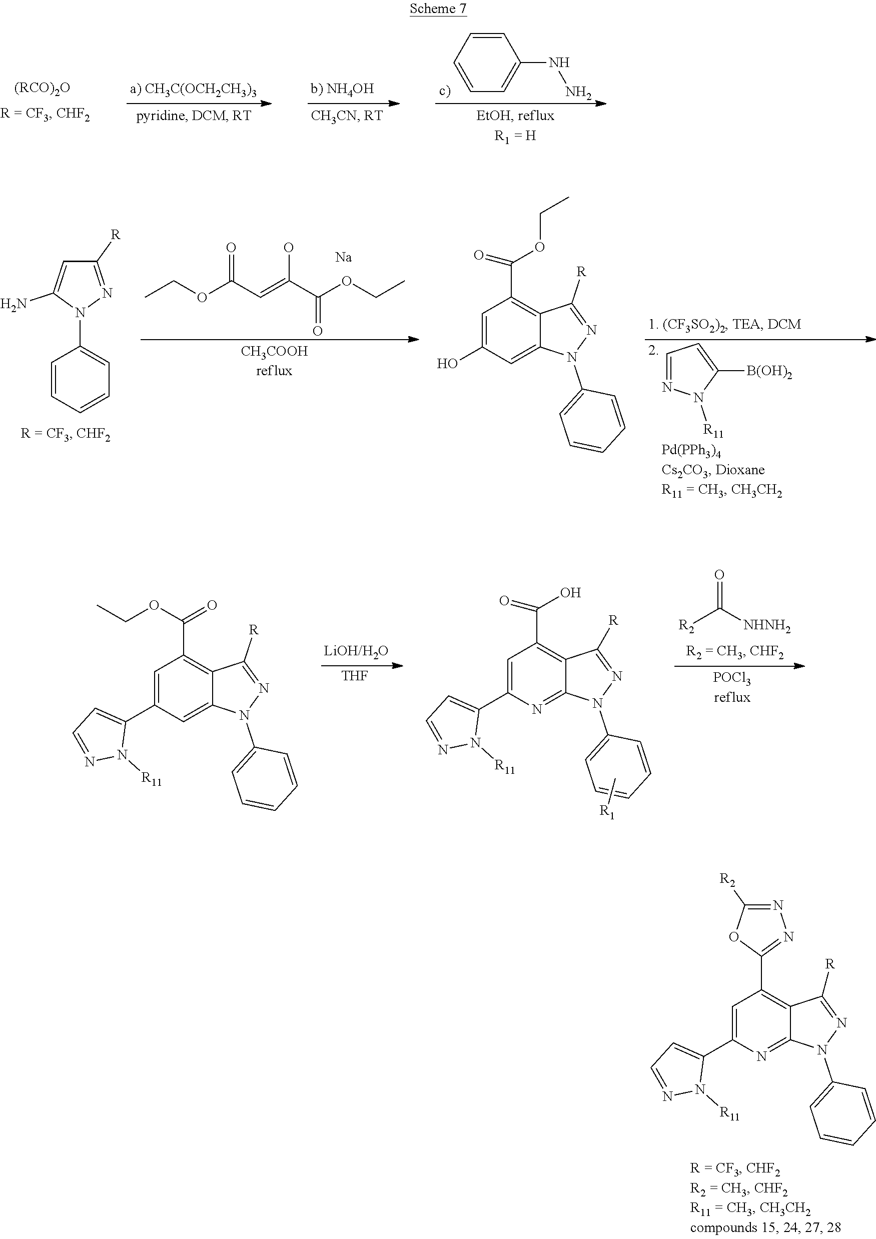

Commercially available aminopyrazoles (e.g. 20 g, 1 equivalent) and sodium diethyl oxaloacetate (e.g. 26.7 g, 1.1 equivalent) were dissolved in acetic acid (250 ml) and heated to reflux overnight. The solvent was removed by rotary evaporation and the product was purified by recrystallization from ethyl acetate to furnish the corresponding pyrazolopyridine product. (e.g. 19 g).

Commercially available hydrazines (e.g. 1b, 5 g) were suspended in 30 ml ethanol to which 1 equivalent of 2-chloroacrylonitrile and 2 equivalents of sodium acetate were added. The mixture was heated to reflux under nitrogen overnight. The cooled reaction was diluted with ice water and neutralized to pH at least 7 with saturated sodium bicarbonate solution, then extracted with three portions of ethyl acetate. The collected organic extracts were dried and concentrated and the crude product purified by silica gel chromatography eluting with ethyl acetate/hexanes to furnish the product aminopyrazole. This aminopyrazole was dissolved in acetic acid and reacted with 1.1 equivalent of sodium diethyl oxaloacetate at reflux overnight. The cooled reaction mixture was poured over ice and the precipitated product was collected by filtration and washed with water then dried.

Intermediate hydroxy pyrazolopyridine (lg) was dissolved in dichloromethane (25 ml) and cooled to 0° C. under nitrogen. Pyridine (2 equivalents) was added, followed dropwise by 1.2 equivalents of trifluoromethyl sulfonic anhydride. The reaction was stirred at 0° C. for three hours whereupon the reaction was diluted with 25 ml dichloromethane and washed with 2 portions of 1M aqueous HCl and brine. The organic extract was dried and concentrated and used without further purification for subsequent reactions.

One gram of this crude triflate was dissolved in 20 ml dioxane at room temperature. Three equivalents of pyrazole boronic acid was added, followed by five equivalents of cesium carbonate. The mixture was purged for several minutes with nitrogen at room temperature, then 0.1 equivalent of Pd(PPh₃)₄ was added. The reaction mixture was then heated to reflux under a nitrogen atmosphere overnight. The cooled reaction was poured into ice water and extracted with 3 portions of ethyl acetate. The collected organic extract was washed with water and brine, then dried, concentrated and purified by silica gel chromatography eluting with hexanes/ethyl acetate to furnish the desired product.

Esters were hydrolyzed to the corresponding carboxylic acids as follows: Ester was dissolved in aqueous tetrahydrofuran at room temperature. Three equivalents of lithium hydroxide were added and the mixture stirred at room temperature overnight. The reaction was poured into ice water and pH was adjusted to 1 with 1N HCl. The resulting precipitate was collected by filtration and dried to furnish the desired carboxylic acids.

### Synthesis of Oxa- and Thio-(3,4-Diazoles)

Carboxylic acid (0.1 g) was dissolved in 3 ml POCl₃ at room temperature. 26 mg (1.2 equivalents) of acetyl hydrazine was added and the reaction was heated to reflux under nitrogen for 3 hours. The cooled reaction was poured into ice water and the pH adjusted to at least 7 with TN NaOH. The aqueous solution was extracted with three portions of ethyl acetate and the collected organic layer was washed with water and brine. The crude product was purified by silica gel chromatography eluting with ethyl acetate/hexanes.

### Synthesis of Methyl Oxazoles

Carboxylic acid (0.1 g) was dissolved in 5 ml DMF at room temperature. 1.2 equivalents of HATU, 4 equivalents of diisopropyl ethylamine and 1.2 equivalents of acetyl or propionyl hydrazine were added. The reaction mixture was stirred at room temperature overnight. The reaction was poured into 1 N HCl and extracted with 3 portions of ethyl acetate. The collected organic extracts were washed with brine, dried and the crude product was purified by silica gel chromatography eluting with ethyl acetate/hexanes.

Carboxylic acid 4 (0.lg) was reacted as described for 12, substituting 1-aminopropanone HCl for the hydrazine. The crude product was dissolved in POCl₃ and heated to reflux for three hours. The reaction was worked up as described above and purified by silica gel chromatography eluting with ethyl acetate/hexanes.

### Synthesis of Methyl Thiazoles

As described for the first step in the synthesis of the corresponding methyl oxazoles above, the crude product was treated with Lawesson's Reagent (2 equivalents) in THF at reflux overnight. The reaction was worked up as described for 10 and the crude product purified by silica gel chromatography eluting with ethyl acetate/hexanes.

### 1. Procedure for the Synthesis of 3-Methyl-2,4 Oxadiazole

SOCl₂ (2 ml) was added into respective carboxylic acid (0.27 mmol, 1 eq) and stirred at 80° C. for four hours. Then excess SOCl₂ was evaporated. Then dioxane added into reaction mixture. To the solution of acid chloride in dioxane, acetamide oxime (0.35 mmol, 1.3 eq) and pyridine (1.3 mmol, 5 eq) was added respectively. White precipitates formed which became dissolved upon heating. Reaction mixture was stirred at 90° C. for overnight and then poured into ice cold water. Precipitates obtained, washed with water, and dried. Pure compound was obtained by flash column using Hexane: Ethyl acetate system. 55-60% yield.

### 2. Procedure for the Synthesis of 5-Methyl-2,4 Oxadiazole

i) Nitrile Formation from Amide

POCl₃ (3 ml) was added into acid amide in a one neck round bottom flask and refluxed the reaction mixture for four hours. Poured into ice cold water and neutralized with NaHCO₃, extracted with ethyl acetate, washed with water, brine, dried with sodium sulfate and evaporated, then purified by flash column using Hexane: Ethyl acetate system to provide pure solid. 50-60% Yield

### ii) Nitrile to 5-Methyl-2,4 Oxadiazole

To the solution of nitrile (1 eq) in DMF, NH₂OH·HCl (1.3 eq) and TEA (2 eq) was added drop wise and stirred at 90° C. for overnight, then poured into ice cold water. Precipitates formed, filtered, and washed and dried. This crude oxime intermediate was dissolved in dioxane and acetyl chloride (1.2 eq), pyridine (1.2 eq) was added drop wise, stirred at 90° C. for overnight, then quenched the reaction mixture by pouring into ice cold water. Precipitates were obtained, and washed with water and dried. Pure compound was obtained by flash column using hexane: ethyl acetate solvent system. 30% Yield

### 3. Procedure for the Synthesis of 2-Diflouromethyl-3,4 Oxadiazole

2,2-difluoroacetohydrazide (5 eq) was added into a flask containing pyrazolo pyridine acid and POCl₃ at room temperature, then the reaction mixture was refluxed overnight. The reaction mixture was then poured into ice cold water, extracted with ethyl acetate, washed with water, and brine, dried with sodium sulfate and evaporated, then purified by flash column using Hexane: Ethyl acetate system to get pure solid. 25-30% Yield

### 4. Procedure for the Synthesis of 3,4 Oxadiazole

To the hot solution of corresponding pyrazolo pyridine ester (1 eq) in dry ethanol was added hydrazine hydrate (5 eq) and refluxed for three hours. Then evaporated the solvent from reaction mixture by rotary evaporator and dried to get the hydrazide quantitatively. Then triethyl orthoformate 3 ml was added to the corresponding hydrazide and refluxed for overnight, poured into ice cold water, then extracted with ethyl acetate, washed with water, brine and dried. Purification was achieved by flash column by using hexane and ethyl acetate as solvent. 25-35% Yield

### Specific Examples

### Experimental

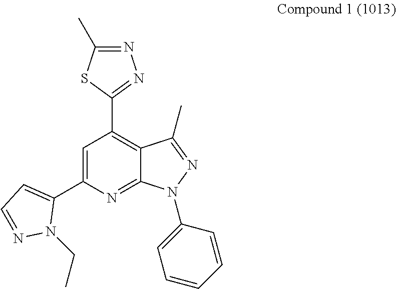 [SMILES: CCn1nccc1-c1cc(-c2nnc(C)s2)c2c(C)nn(-c3ccccc3)c2n1]

¹H NMR (400 MHz, CDCl₃) δ 8.21-8.18 (d, J=8 Hz, 2H), 7.67 (s, 1H), 7.63 (s, 1H), 7.59-7.55 (t, J=11 Hz, 2H), 7.41-7.37 (t, J=7 Hz, 1H), 6.79 (s, 1H), 4.87-4.81 (q, J=7 Hz, 2H), 2.99 (s, 3H), 2.73 (s, 1H), 1.56-1.52 (t, J=7 Hz, 3H).

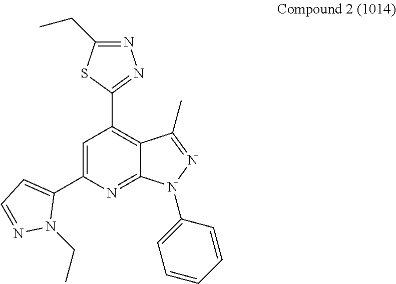 [SMILES: CCc1nnc(-c2cc(-c3ccnn3CC)nc3c2c(C)nn3-c2ccccc2)s1]

¹H NMR (400 MHz, CDCl₃) δ 8.19-8.17 (d, J=8 Hz, 2H), 7.66 (s, 1H), 7.62-7.61 (d, J=1.8 Hz, 1H), 7.57-7.53 (t, J=8 Hz, 2H), 7.39-7.38 (t, J=7.8 Hz, 1H), 6.79-6.78 (d, J=1.8 Hz, 1H), 4.85-4.80 (q, J=7 Hz, 2H), 2.73 (s, 3H), 1.61-1.57 (t, J=7.6 Hz, 3H), 1.55-1.51 (t, J=7.2 Hz, 3H).

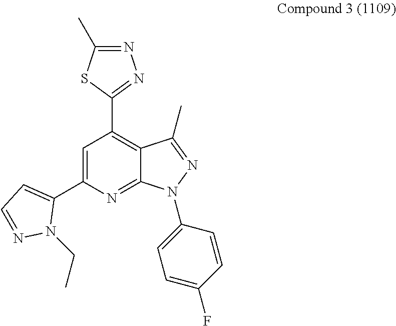 [SMILES: CCn1nccc1-c1cc(-c2nnc(C)s2)c2c(C)nn(-c3ccc(F)cc3)c2n1]

¹H NMR (400 MHz, CDCl₃) δ 8.84 (s, 1H), 8.20-8.16 (m, 2H), 7.91 (s, 1H), 7.65 (d, J=1.9 Hz, 1H), 7.29-7.25 (m, 2H), 6.85-6.84 (d, J=2.0 Hz, 1H), 4.82-4.77 (q, J=7 Hz, 2H), 2.98 (s, 3H), 1.59-1.52 (t, J=7 Hz, 3H).

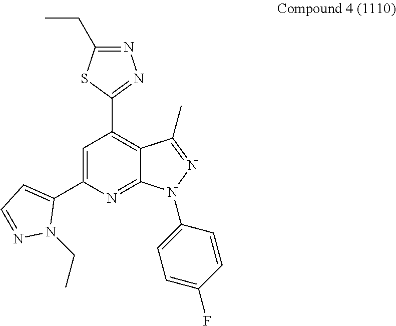 [SMILES: CCc1nnc(-c2cc(-c3ccnn3CC)nc3c2c(C)nn3-c2ccc(F)cc2)s1]

¹H NMR (400 MHz, CDCl₃) (8.91 (s, 1H), 8.23-8.20 (m, 2H), 7.97 (s, 1H), 7.68-7.68 (d, J=2 Hz, 1H), 7.31-7.28 (t, J=8.4 Hz, 2H), 6.88-6.88 (d, J=1.6 Hz, 1H), 4.84-4.82 (q, J=7.2 Hz, 2H), 3.38-3.33 (q, J=7.6 Hz, 2H), 1.63-1.54 (m, 6H).

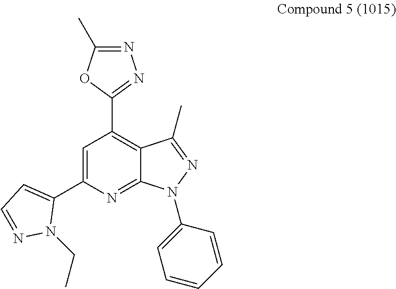 [SMILES: CCn1nccc1-c1cc(-c2nnc(C)o2)c2c(C)nn(-c3ccccc3)c2n1]

¹H NMR (400 MHz, CDCl₃) δ 8.19-8.16 (m, 2H), 8.06 (s, 1H), 7.65-7.64 (d, J=2 Hz, 1H), 7.59-7.55 (m, 2H), 7.42-7.39 (m, 1H), 6.88-6.88 (d, J=2 Hz, 1H), 4.87-4.81 (q, J=7.2 Hz, 2H), 2.96 (s, 3H), 2.79 (s, 3H), 1.55-1.51 (t, J=7.2 Hz, 3H).

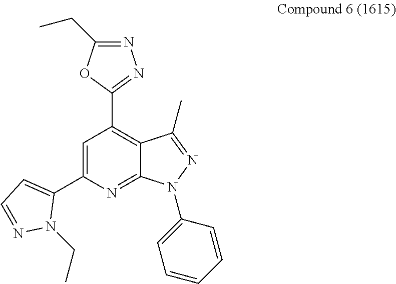 [SMILES: CCc1nnc(-c2cc(-c3ccnn3CC)nc3c2c(C)nn3-c2ccccc2)o1]

¹H NMR (400 MHz, CDCl₃) δ 8.17-8.15 (m, 2H), 8.06 (s, 1H), 7.63-7.63 (d, J=2 Hz, 1H), 7.58-7.54 (m, 2H), 7.41-7.37 (m, 1H), 6.87-6.87 (d, J=2 Hz, 1H), 4.85-4.79 (q, J=7.2 Hz, 2H), 3.13-3.09 (t, J=7.6 Hz, 3H), 2.94 (s, 3H), 1.59-1.50 (m, 6H).

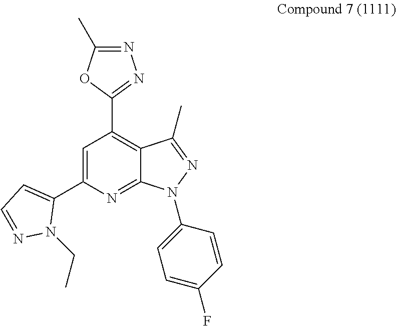 [SMILES: CCn1nccc1-c1cc(-c2nnc(C)o2)c2c(C)nn(-c3ccc(F)cc3)c2n1]

¹H NMR (400 MHz, CDCl₃) δ 8.82 (s, 1H), 8.21-8.17 (m, 2H), 8.14 (s, 1H), 7.67-7.66 (d, J=2 Hz, 1H), 7.31-7.27 (m, 2H), 6.91-6.91 (d, J=1.2 Hz, 1H), 4.85-4.80 (q, J 7.2 Hz, 2H), 2.81 (s, 3H), 1.57-1.53 (t, J=6.8 Hz, 3H).

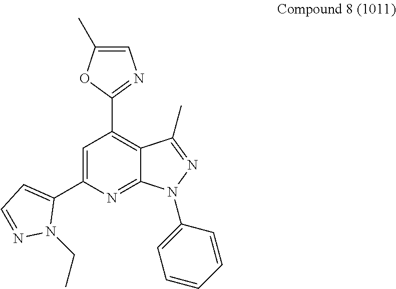 [SMILES: CCn1nccc1-c1cc(-c2ncc(C)o2)c2c(C)nn(-c3ccccc3)c2n1]

¹H NMR (400 MHz, CDCl₃) δ 8.21-8.18 (d, J=8.4 Hz, 2H), 8.08-8.08 (d, J=1.2 Hz, 1H), 7.63-7.63 (m, 1H), 7.58-7.54 (m, 2H), 7.40-7.32 (m, 1H), 7.09 (s, 1H), 6.88-6.87 (d, J=1.6 Hz, 1H), 4.87-4.82 (q, J=7.2 Hz, 2H), 2.96-2.95 (d, J=0.8 Hz, 3H), 2.55 (s, 3H), 1.55-1.52 (t, J=7.2 Hz, 3H).

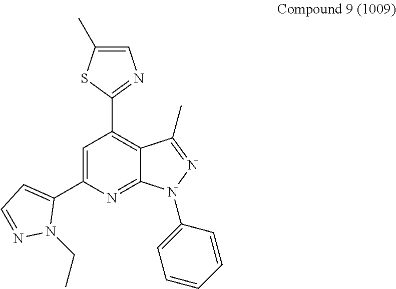 [SMILES: CCn1nccc1-c1cc(-c2ncc(C)s2)c2c(C)nn(-c3ccccc3)c2n1]

¹H NMR (400 MHz, CDCl₃) δ 8.22-8.20 (m, 2H), 7.79 (s, 1H), 7.68-7.65 (m, 1H), 7.59-7.55 (m, 2H), 7.40-7.38 (t, J=7.6 Hz, 2H), 6.82 (s, 1H), 4.89-4.84 (q, J=7.2 Hz, 2H), 2.76 (s, 3H), 2.68-2.67 (d, J=0.8 Hz, 3H), 1.57-1.54 (t, J=7.2 Hz, 3H).

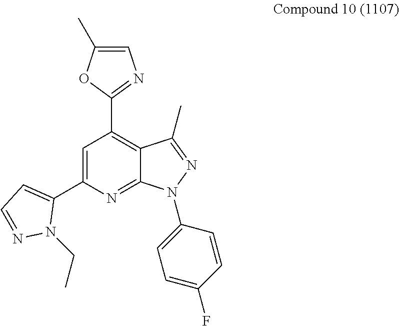 [SMILES: CCn1nccc1-c1cc(-c2ncc(C)o2)c2c(C)nn(-c3ccc(F)cc3)c2n1]

¹H NMR (400 MHz, CDCl₃) δ 8.82 (s, 1H), 8.23-8.20 (m, 2H), 8.14 (s, 1H), 7.66 (s, 1H), 7.31-7.26 (m, 3H), 7.14 (s, 1H), 6.91 (s, 1H), 4.86-4.81 (q, J=6.8 Hz, 2H), 2.59 (s, 3H), 1.57-1.54 (t, J=6.8 Hz, 3H).

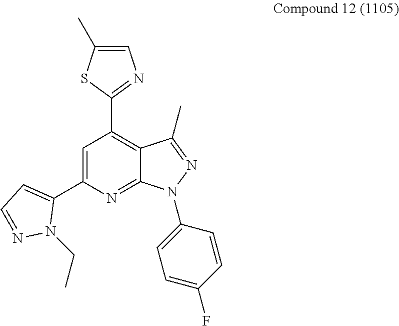 [SMILES: CCn1nccc1-c1cc(-c2ncc(C)s2)c2c(C)nn(-c3ccc(F)cc3)c2n1]

¹H NMR (400 MHz, CDCl₃) δ 8.79 (s, 1H), 8.20-8.17 (m. 2H), 7.91 (s, 1H), 7.76-7.76 (d, J=0.8 Hz, 1H), 7.64-7.63 (d, J=2 Hz, 1H), 7.27-7.23 (m, 2H), 6.83-6.83 (d, J=2 Hz, 1H), 4.82-4.77 (q, J=7.2 Hz, 2H), 2.65-2.64 (d, J=0.8 Hz, 3H), 1.56-1.52 (t, J=7.2 Hz, 3H).

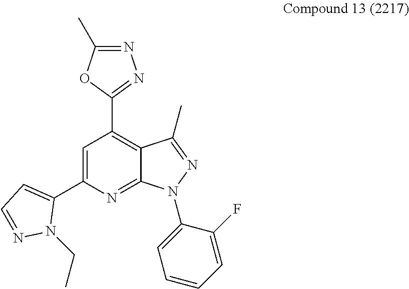 [SMILES: CCn1nccc1-c1cc(-c2nnc(C)o2)c2c(C)nn(-c3ccccc3F)c2n1]

¹H NMR (400 MHz, CDCl₃) δ 8.04 (s, 1H), 7.70-7.65 (m, 1H), 7.57-7.56 (d, J=2 Hz, 1H), 7.51-7.47 (m, 1H), 7.37-7.32 (m, 2H), 6.83-6.83 (d, J=2 Hz, 1H), 4.68-4.66 (q, J=7.2 Hz, 2H), 2.95 (s, 3H), 2.77 (s, 3H), 1.34-1.31 (t, J=7.2 Hz, 3H).

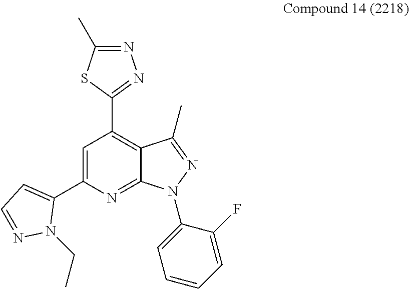 [SMILES: CCn1nccc1-c1cc(-c2nnc(C)s2)c2c(C)nn(-c3ccccc3F)c2n1]

¹H NMR (400 MHz, CDCl₃) δ 7.69-7.66 (m, 2H), 7.57-7.56 (d, J=2 Hz, 1H), 7.50-7.47 (m, 1H), 7.37-7.32 (m, 2H), 6.76-6.75 (d, J=2 Hz, 1H), 4.71-4.66 (q, J=7.2 Hz, 2H), 2.97 (s, 3H), 2.74 (s, 3H), 1.36-1.33 (t, J=7.2 Hz, 3H).

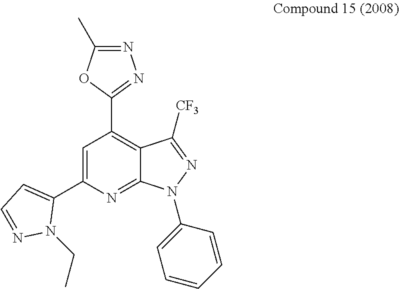 [SMILES: CCn1nccc1-c1cc(-c2nnc(C)o2)c2c(C(F)(F)F)nn(-c3ccccc3)c2n1]

¹H NMR (400 MHz, CDCl₃) δ 8.37 (s, 1H), 8.15-8.12 (m, 2H), 7.68-7.65 (m, 3H), 7.63-7.54 (m, 1H), 6.99 (s, 1H), 4.87-4.83 (q, J=7.2 Hz, 2H), 2.79 (s, 3H), 1.52-1.49 (t, J=7.2 Hz, 3H).

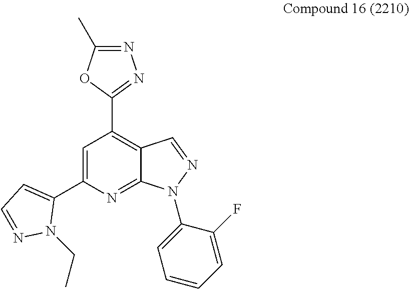 [SMILES: CCn1nccc1-c1cc(-c2nnc(C)o2)c2cnn(-c3ccccc3F)c2n1]

¹H NMR (400 MHz, CDCl₃) (8.81 (s, 1H), 8.27 (s, 1H), 8.74-7.72 (t, J=6 Hz, 1H), 7.62-7.62 (d, J=2 Hz, 1H), 7.58-7.52 (m, 1H), 7.42-7.36 (m, 2H), 6.89-6.88 (d, J=2 Hz, 1H), 4.75-4.70 (q, J=7.2 Hz, 2H), 2.81 (s, 3H), 1.40-1.36 (t, J=7.2 Hz, 3H).

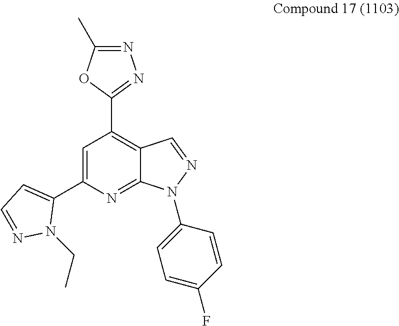 [SMILES: CCn1nccc1-c1cc(-c2nnc(C)o2)c2cnn(-c3ccc(F)cc3)c2n1]

¹H NMR (400 MHz, CDCl₃) δ 8.14-8.10 (m, 2H), 8.05 (s, 1H), 7.65-7.64 (d, J=2 Hz, 1H), 7.28-7.23 (m, 2H), 6.87-6.87 (d, J=2.4 Hz, 1H), 4.83-4.77 (q, J=7.2 Hz, 2H), 2.94 (s, 3H), 2.79 (s, 3H), 1.53-1.49 (t, J=7.2 Hz, 3H).

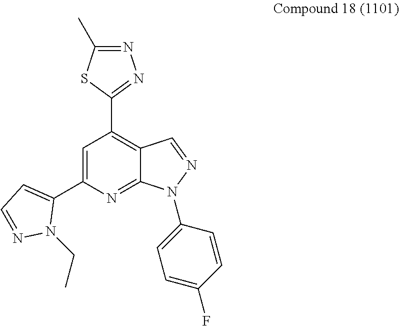 [SMILES: CCn1nccc1-c1cc(-c2nnc(C)s2)c2cnn(-c3ccc(F)cc3)c2n1]

¹H NMR (400 MHz, CDCl₃) δ 8.13-8.09 (m, 2H), 7.63 (s, 1H), 7.60-7.59 (d, J=2 Hz, 1H), 7.24-7.20 (m, 2H), 6.76-6.75 (d, J=2.0 Hz, 1H), 4.79-4.74 (q, J=6.8 Hz, 2H), 2.96 (s, 3H), 2.69 (s, 3H), 1.52-1.48 (t, J=7.2 Hz, 3H).

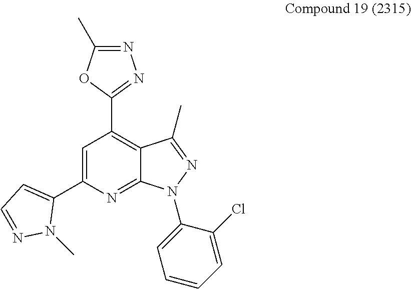 [SMILES: Cc1nnc(-c2cc(-c3ccnn3C)nc3c2c(C)nn3-c2ccccc2Cl)o1]

¹H NMR (400 MHz, CDCl₃) δ 8.07 (s, 1H), 7.67-7.65 (m, 1H), 7.63-7.61 (m, 1H), 7.57-7.56 (d, J=2 Hz, 1H), 7.54-7.50 (m, 2H), 6.88-6.87 (d, J=2.0 Hz, 1H), 4.19 (s, 3H), 2.98 (s, 3H), 2.79 (s, 3H).

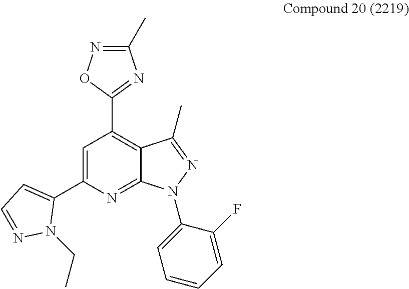 [SMILES: CCn1nccc1-c1cc(-c2nc(C)no2)c2c(C)nn(-c3ccccc3F)c2n1]

¹H NMR (400 MHz, CDCl₃) δ 8.20 (s, 1H), 7.72-7.68 (m, 1H), 7.60-7.59 (d, J=5.2 Hz, 1H), 7.55-7.50 (m, 1H), 7.40-7.34 (m, 2H), 6.88 (s, 1H), 4.72-4.67 (q, J=6.8 Hz, 2H), 2.95 (s, 3H), 2.63 (s, 3H), 1.36-1.33 (t, J=7.2 Hz, 3H).

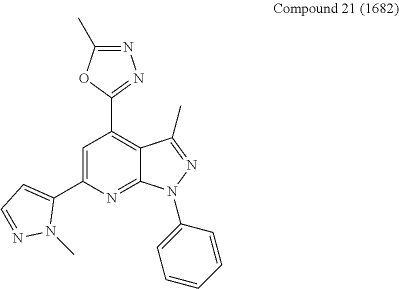 [SMILES: Cc1nnc(-c2cc(-c3ccnn3C)nc3c2c(C)nn3-c2ccccc2)o1]

¹H NMR (400 MHz, CDCl₃) δ 8.23-8.21 (m, 2H), 8.07 (s, 1H), 7.65-7.65 (d, J=2 Hz, 1H), 7.61-7.57 (m, 2H), 7.43-7.41 (m, 1H), 6.92-6.91 (d, J=2 Hz, 1H), 4.41 (s. 3H), 2.97 (s, 3H), 2.80 (s, 3H).

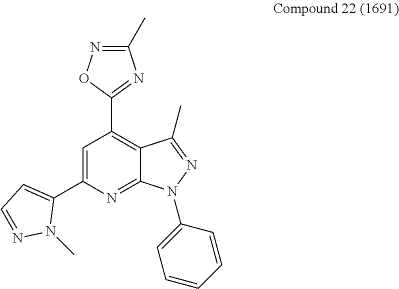 [SMILES: Cc1noc(-c2cc(-c3ccnn3C)nc3c2c(C)nn3-c2ccccc2)n1]

¹H NMR (400 MHz, CDCl₃) δ 8.18-8.16 (m, 2H), 8.10 (s, 1H), 7.59-7.53 (m, 3H), 7.40-7.36 (m, 1H), 6.87-6.86 (d, J=2 Hz, 1H), 4.35 (s, 3H), 2.88 (s, 3H), 2.61 (s, 3H).

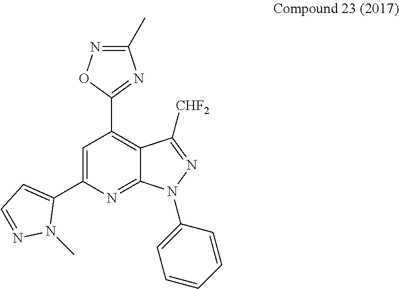 [SMILES: Cc1noc(-c2cc(-c3ccnn3C)nc3c2c(C(F)F)nn3-c2ccccc2)n1]

¹H NMR (400 MHz, CDCl₃) δ 8.34 (s, 1H), 8.22-8.20 (m, 2H), 8.05-7.78 (t, J=54 Hz, 1H), 7.67-7.62 (m, 3H), 7.53-7.49 (m, 1H), 7.0 (s, 1H), 4.41 (s, 3H), 2.67 (s, 3H).

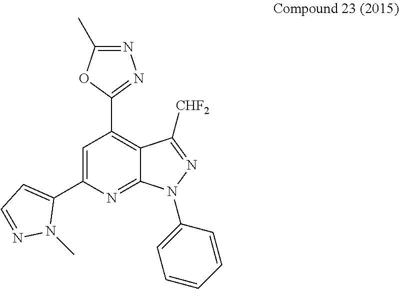 [SMILES: Cc1nnc(-c2cc(-c3ccnn3C)nc3c2c(C(F)F)nn3-c2ccccc2)o1]

¹H NMR (400 MHz, CDCl₃) δ 8.22-8.19 (m, 3H), 8.00-7.74 (t, J=53.6 Hz, 1H), 7.65-7.61 (m, 3H), 7.52-7.50 (m, 1H), 6.90 (s, 1H), 4.39 (s, 3H), 2.80 (s, 3H).

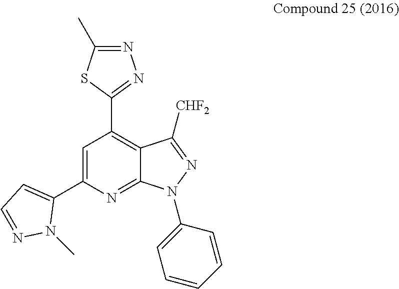 [SMILES: Cc1nnc(-c2cc(-c3ccnn3C)nc3c2c(C(F)F)nn3-c2ccccc2)s1]

¹H NMR (400 MHz, CDCl₃) δ 8.23-8.20 (m, 2H), 7.90-7.76 (m, 2H), 7.65-7.60 (m, 3H), 7.50-7.47 (m, 1H), 6.88-6.88 (d, J=2 Hz, 1H), 4.39 (s, 3H), 3.00 (s, 3H).

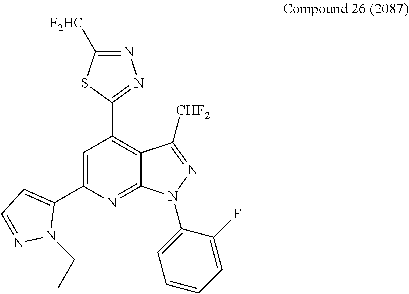 [SMILES: CCn1nccc1-c1cc(-c2nnc(C(F)F)s2)c2c(C(F)F)nn(-c3ccccc3F)c2n1]

¹H NMR (400 MHz, CDCl₃) δ 8.19 (s, 1H), 7.72-7.69 (m, 1H), 7.61-7.61 (d, J=2 Hz, 1H), 7.57-7.51 (m, 1H), 7.41-7.35 (m, 2H), 7.22-6.96 (t, J=51.6 Hz, 1H), 6.90-6.89 (d, J=1.6 Hz, 1H), 4.72-4.67 (q, J=7.2 Hz, 2H), 2.97 (s, 3H), 1.36-1.33 (t, J=7.2 Hz, 3H).

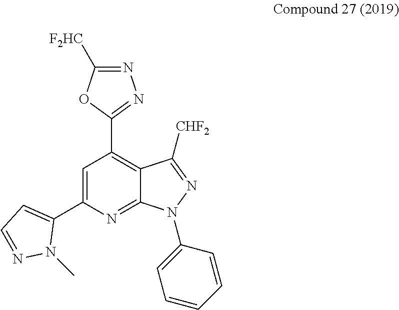 [SMILES: Cn1nccc1-c1cc(-c2nnc(C(F)F)o2)c2c(C(F)F)nn(-c3ccccc3)c2n1]

¹H NMR (400 MHz, CDCl₃) δ 8.30 (s, 1H), 8.10-8.09 (m, 2H), 7.85-7.52 (m, 5H), 7.21-6.95 (m, 2H), 4.40 (s, 3H).

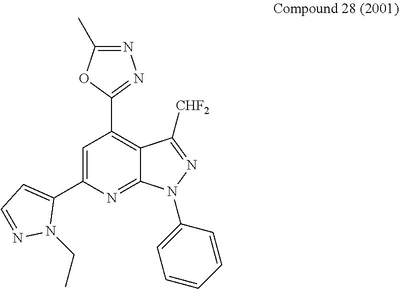 [SMILES: CCn1nccc1-c1cc(-c2nnc(C)o2)c2c(C(F)F)nn(-c3ccccc3)c2n1]

¹H NMR (400 MHz, CDCl₃) δ 8.18 (s, 1H), 8.10-8.09 (m, 2H), 8.00-7.73 (t, J=53.6 Hz, 1H), 7.62-7.56 (m, 3H), 7.49-7.45 (m, 1H), 6.91-6.90 (d, J=2 Hz, 1H), 4.81-4.75 (q, J=7.2 Hz, 2H), 2.78 (s, 3H), 1.50-1.46 (t, J=7.2 Hz, 3H),

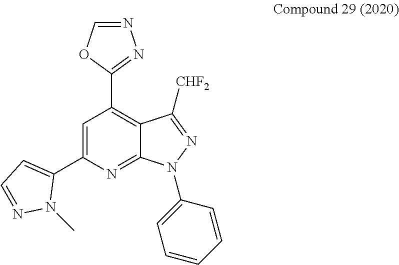 [SMILES: Cn1nccc1-c1cc(-c2nnco2)c2c(C(F)F)nn(-c3ccccc3)c2n1]

¹H NMR (400 MHz, CDCl₃) δ 8.77 (s, 1H), 8.27 (s, 1H), 8.22-8.19 (m, 2H), 8.00-7.73 (t, J=53.6 Hz, 1H), 7.67-7.62 (m, 3H), 7.53-7.51 (m, 1H), 6.97-6.96 (d, J=2 Hz, 1H), 4.40 (s, 3H).

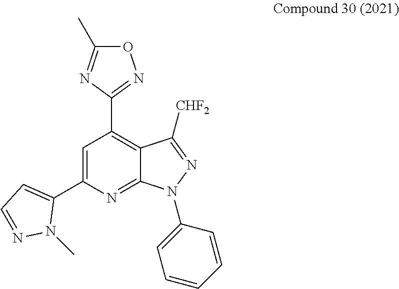 [SMILES: Cc1nc(-c2cc(-c3ccnn3C)nc3c2c(C(F)F)nn3-c2ccccc2)no1]

¹H NMR (400 MHz, CDCl₃) δ 8.31 (s, 1H), 8.23-8.21 (m, 2H), 8.01-7.74 (t, J=54 Hz, 1H), 7.65-7.60 (m, 3H), 7.51-7.47 (m, 1H), 6.97-6.97 (d, J=2 Hz, 1H), 4.40 (s, 3H), 2.83 (s, 3H).

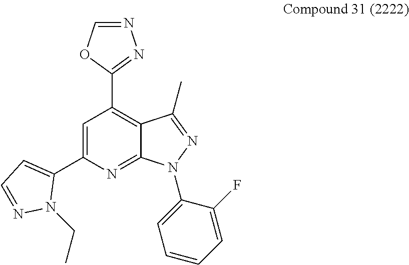 [SMILES: CCn1nccc1-c1cc(-c2nnco2)c2c(C)nn(-c3ccccc3F)c2n1]

¹H NMR (400 MHz, CDCl₃) δ 8.76 (s, 1H), 8.13 (s, 1H), 7.72-7.70 (m, 1H), 7.68-7.68 (d, J=2 Hz, 1H), 7.62-7.52 (m, 1H), 7.41-7.34 (m, 2H), 6.86-6.86 (d, J=2 Hz, 1H), 4.72-4.70 (q, J=6.8 Hz, 2H), 2.99 (s, 3H), 1.37-1.34 (t, J=7.2 Hz, 3H).

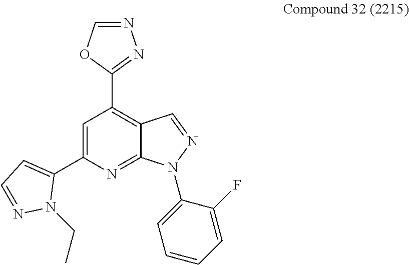 [SMILES: CCn1nccc1-c1cc(-c2nnco2)c2cnn(-c3ccccc3F)c2n1]

¹H NMR (400 MHz, CDCl₃) δ 8.89 (s, 1H), 8.78 (s, 1H), 8.21 (s, 1H), 7.74-7.72 (m, 1H), 7.63-7.62 (d, J=2 Hz, 1H), 7.57-7.53 (m, 1H), 7.43-7.36 (m, 2H), 6.89-6.88 (d, J=2 Hz, 1H), 4.75-4.71 (q, J=7.2 Hz, 2H), 1.40-1.36 (t, J=7.2 Hz, 3H).

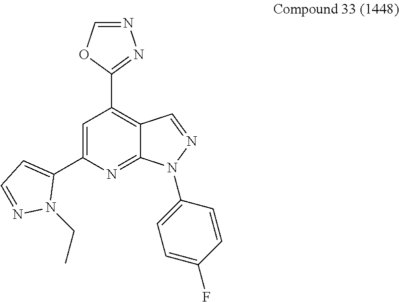 [SMILES: CCn1nccc1-c1cc(-c2nnco2)c2cnn(-c3ccc(F)cc3)c2n1]

¹H NMR (400 MHz, CDCl₃) δ 8.82 (s, 1H), 8.76 (s, 1H), 8.20 (s, 1H), 8.19-8.15 (m, 2H), 7.66-7.65 (d, J=2 Hz, 1H), 7.30-7.26 (m, 2H), 6.90-6.89 (d, J=2 Hz, 1H), 4.84-4.78 (q, J=7.2 Hz, 2H), 1.55-1.52 (t, J=7.2 Hz, 3H).

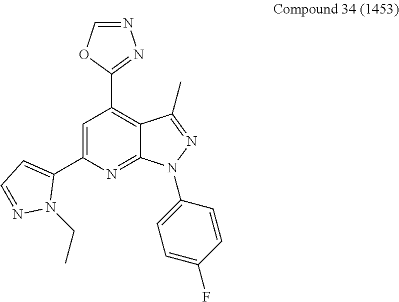 [SMILES: CCn1nccc1-c1cc(-c2nnco2)c2c(C)nn(-c3ccc(F)cc3)c2n1]

¹H NMR (400 MHz, CDCl₃) δ 8.75 (s, 1H), 8.15-8.11 (m, 3H), 7.66-7.66 (d, J=2 Hz, 1H), 7.29-7.25 (m, 2H), 6.88-6.88 (d, J=2 Hz, 1H), 4.84-4.79 (q, J=7.2 Hz, 2H), 2.96 (s, 3H), 1.54-1.51 (t, J 7.2 Hz, 3H).

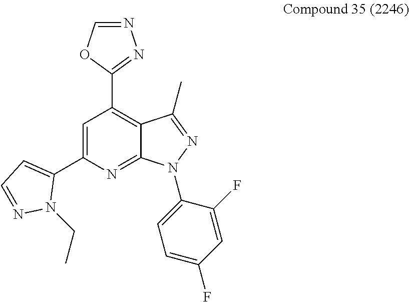 [SMILES: CCn1nccc1-c1cc(-c2nnco2)c2c(C)nn(-c3ccc(F)cc3F)c2n1]

¹H NMR (400 MHz, CDCl₃) δ 8.76 (s, 1H), 8.12 (s, 1H), 7.68-7.65 (m, 1H), 7.60-7.59 (d, J=2 Hz, 1H), 7.15-7.09 (m, 2H), 6.85-6.84 (d, J=2 Hz, 1H), 4.70-4.65 (q, J=6.8 Hz, 2H), 2.96 (s, 3H), 1.37-1.34 (t, J=7.2 Hz, 3H).

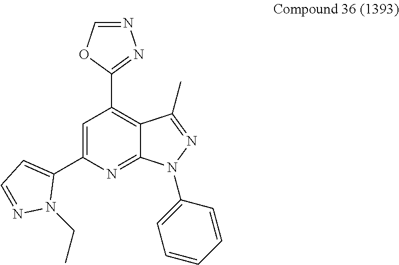 [SMILES: CCn1nccc1-c1cc(-c2nnco2)c2c(C)nn(-c3ccccc3)c2n1]

¹H NMR (400 MHz, CDCl₃) δ 8.74 (s, 1H), 8.17-8.15 (m, 2H), 8.10 (s, 1H), 7.65-7.64 (d, J=2 Hz, 1H), 7.59-7.55 (m, 2H), 7.42-7.40 (m, 1H), 6.87-6.87 (d, J=2 Hz, 1H), 4.86-4.81 (q, J=7.2 Hz, 2H), 2.95 (s, 3H), 1.55-1.30 (t, J=7.2 Hz, 3H).

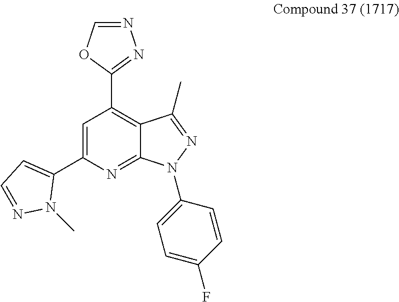 [SMILES: Cc1nn(-c2ccc(F)cc2)c2nc(-c3ccnn3C)cc(-c3nnco3)c12]

¹H NMR (400 MHz, CDCl₃) δ 8.76 (s, 1H), 8.20-8.16 (m, 2H), 8.14 (s, 1H), 7.69-7.68 (d, J=2 Hz, 1H), 7.29-7.27 (m, 2H), 6.93-6.93 (d, J=2 Hz, 1H), 4.42 (s, 3H), 2.98 (s, 3H).

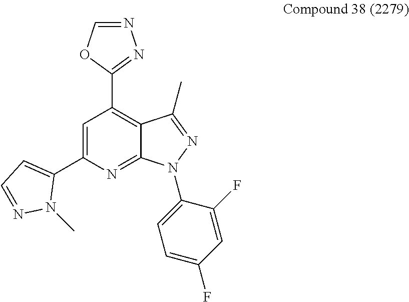 [SMILES: Cc1nn(-c2ccc(F)cc2F)c2nc(-c3ccnn3C)cc(-c3nnco3)c12]

¹H NMR (400 MHz, CDCl₃) δ 8.76 (s, 1H), 8.15 (s, 1H), 7.73-7.67 (m, 2H), 7.14-7.12 (m, 2H), 6.91-6.90 (d, J=2 Hz, 1H), 4.30 (s, 3H), 2.99 (s, 3H).

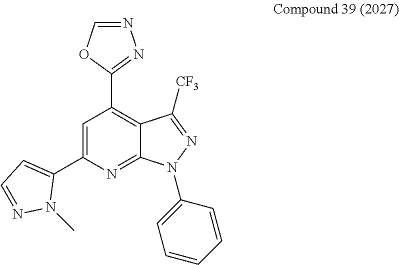 [SMILES: Cn1nccc1-c1cc(-c2nnco2)c2c(C(F)(F)F)nn(-c3ccccc3)c2n1]

¹H NMR (400 MHz, CDCl₃) δ 8.79 (s, 1H), 8.34 (s, 1H), 8.20-8.17 (m, 2H), 7.67-7.63 (m, 3H), 7.56-7.54 (m, 1H), 6.98-6.98 (d, J=2 Hz, 1H), 4.39 (s, 3H).

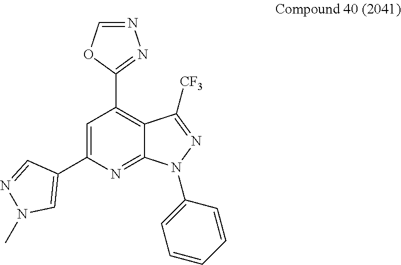 [SMILES: Cn1cc(-c2cc(-c3nnco3)c3c(C(F)(F)F)nn(-c4ccccc4)c3n2)cn1]

¹H NMR (400 MHz, CDCl₃) δ 8.76 (s, 1H), 8.30-8.29 (m, 2H), 8.22-8.15 (m, 3H), 7.68-7.64 (m, 2H), 7.53-7.49 (m, 1H), 4.09 (s, 3H).

### Example 6: Optimization of PDE11A4 Inhibitors for A2e-Related Cognitive Decline

Compounds must be cell permeable and have sufficient aqueous solubility, features essential for PDE11A inhibition and drug candidates in general. Four different chemotypes (4-7) that demonstrated PDE selectivity and sub-micromolar PDE11A potency are shown in FIG. 27. The in vitro ADME properties of 4-7 were investigated and, as shown in Table 2, each compound had positive and negative attributes. Tricycle 6 (FIG. 27) has good water solubility and generally favorable metabolic stability in human and mouse microsomes with a favorable CYP profile. However, it has fewer obvious vectors and a screen of commercially available analogs furnished flat structure-activity data. Pyrrolopyrimidine 5 (FIG. 27) is highly lipophilic with poor water solubility, a clean CYP profile and good metabolic stability with three sites for optimization. Thienopyrimidinone 7 (FIG. 27) is metabolically unstable and analysis of commercially available propylthio analogs were significantly less potent (>1 μM). While not wishing to be bound by any particular theory, this result suggests that this site would have limited value. Pyrazolopyridine 4 (FIG. 27) showed rapid oxidative metabolism with good water solubility, is not a substrate for the p-glycoprotein pump with a favorable CYP profile, and has at least four sites that can be investigated to address these deficiencies.

TABLE 2
Screening hits in vitro ADME Parameters

| | pH 7.4 PBS | Mouse liver | Hu liver | | | | MDCK |
|---|---|---|---|---|---|---|---|
| | solubility | microsomal | microsomal | IC₅₀ (μM) | IC₅₀ (μM) | IC₅₀ (μM) | efflux |
| Cpd | (μM) | t_{1/2} (min) | t_{1/2} (min) | Hu CYP3A4 | Hu CYP2D6 | Hu CYP2C9 | ratio |
| 4 | 54 | <2 | <2 | 3 | >10 | >10 | 0.75 |
| 5 | 3 | 45 | 31 | >10 | >10 | >10 | NT |
| 6 | 150 | 18 | >60 | 1100 | >10 | >10 | NT |
| 7 | 25 | <2 | 12 | >10 | >10 | >10 | NT |

### Results

To determine if these PDE11A4 inhibitors are active in cells and whether enzymatic potency translates in a cellular system, a murine HT-22 hippocampal cell line that does not express PDE11A4 was transfected to express either green fluorescent protein as a negative control or human PDE11 A4. This permitted examination of PDE11 A4-mediated cGMP and cAMP hydrolysis. Using tadalafil (1) as a positive control, compounds 14b and 23b were selected because 14b is comparable to 1 in enzymatic potency and difluoromethyl analog 23b is measurably more potent in the biochemical assay compared to 14b. Concentration-dependent effects of all three compounds on both cyclic nucleotides were observed to furnish EC₅₀ values shown in Table 6. Tadalafil (1) and 14b showed similar efficacy, and compound 23b showed a significant improvement in efficacy compared to 1 for both cAMP and cGMP. While not wishing to be bound by any particular theory, this resulting improvement, relative to tadalafil is hypothesized to unlikely be associated with enzymatic potency, given the similarity between the 23b and 1. All three compounds exhibited comparable effects on both cyclic nucleotide substrates, providing cellular validation of the biochemical results mentioned above. The effects of these PDE11 inhibitors are not due to protein expression or cytotoxicity. This experiment provides an in vitro demonstration of the beneficial effect of PDE11A4 inhibition in a relevant neuronal cell type.

TABLE 6
Cell-based PDE activity of 1 (tadalafil), 14b (3007) and 23b (3030).
(EC₅₀ values represent the average of four independent experiments)

| Cpd | EC₅₀ (μM) cAMP | EC₅₀ (μM) cGMP |
|---|---|---|
| 1 (tadalafil) | 17 | 17 |
| 14b (3007) | 4.7 | 12 |
| 23b (3030) | 2.4 | 1.7 |

### In Vitro ADME

Preliminary metabolite identification was carried out to investigate site(s) for oxidative degradation of 14b. Based on LCMS/MS data of metabolites derived from microsomal incubation, the diethyl amide was identified as a primary site for oxidative transformation. Accordingly, perdeutero analog 25 was analyzed. As shown in Table 7, 25 had a small improvement in microsomal stability. The primary site of metabolism in 25 was the deuterated alkyl groups on the amide, as evidenced by loss of 18 mass units (CD3) in the primary metabolite. Subsequent metabolism of 25 occurred in this region of the compound.

TABLE 7
In vitro ADME parameters for selected PDE11A4 inhibitors

| | pH 7.4 PBS | Mouse liver | Hu liver | | | |
|---|---|---|---|---|---|---|
| | solubility | microsomal | microsomal | IC₅₀ (μM) | IC₅₀ (μM) | IC₅₀ (μM) |
| Cpd | (μM) | t_{1/2} (min) | t_{1/2} (min) | Hu CYP3A4 | Hu CYP2D6 | Hu CYP2C9 |
| 14b (3007) | 135 | 2.3 | 3.2 | 10 | 1.4 | 4.4 |
| 14c (3013) | 120 | 1.9 | 4.2 | >10 | >10 | 1.5 |
| 23b (3030) | 42 | 3.6 | 4.3 | 5.8 | >10 | 5.6 |
| 25 | 39 | 5.2 | 8 | 7.7 | >10 | >10 |

Difluoromethyl amide 23b like 4 and 14b, did not show efflux potential in MDCK-MDR1 cell culture, with an efflux ratio of 0.73. This change resulted in a decrease in kinetic water solubility compared to 14b to less than 50 micromolar. There was no change in CYP2D6 or 2C₉ inhibition, and a small increase in CYP3A4 activity. Deuteration of the alkyl groups improved the CYP profile of 25, resulting in a significant decrease in CYP2C₉ inhibition and a small decrease in CYP3A4 inhibition.

### Experimental Section

### Compound Characterization:

All reagents and solvents were used as received from commercial suppliers. All reactions were dried with sodium sulfate and carried out under a nitrogen atmosphere unless otherwise stated. Compounds were analyzed using a CEM mini LC system with a Restek-C_{18 5} μm column (150 mm×4.6 mm, 80% acetonitrile/water isocratic gradient over 6 minutes with UV detection at 254 nm). Thin layer chromatography was done on silica gel G plates with UV detection. All of the reported yields are for isolated products and compounds were purified by automated flash chromatography (Teledyne Isco Rf200+). Proton NMR spectra were obtained at 400 MHz in CDCl₃ unless otherwise stated.

### Synthetic Procedures

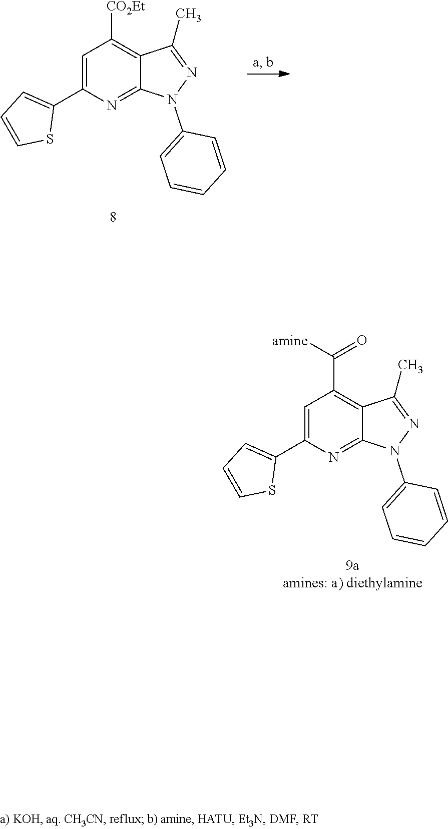

Pyrazolopyridine ester 8 was synthesized as described. Ester hydrolysis was carried out using two equivalents of LiOH in 25% aqueous THF at room temperature overnight. Following evaporation of THF, the pH was adjusted to 2 by pH paper to deposit a solid that was collected by filtration, washed with cold water and dried to provide an off white solid that was used without further purification.

The carboxylic acid (1 eq.) was dissolved in DMF at room temperature and 3 equivalents of triethylamine was added followed by HATU (2 eq.) and an appropriate amine (2 eq.). The reaction stirred at room temperature overnight and was poured into ice water and extracted with three portions of ethyl acetate. The combined organic layer was washed with two portions of 1N HCl and brine, dried and concentrated by rotary evaporation. Purification by silica gel chromatography eluting with hexanes/ethyl acetate provided pure products.

9a (3001): yield 61%, ¹H NMR (400 MHz, CDCl₃) δ 8.37-8.35 (d, J=8 Hz, 2H), 7.72-7.71 (d, J=3.6 Hz, 1H), 7.55-7.51 (t, J=7.6 Hz, 2H), 7.47-7.46 (d, J=5.2 Hz, 1H), 7.41 (s, 1H), 7.29-7.27 (t, J=7.4 Hz, 1H), 7.14 (m, 1H), 3.73 (br, 2H), 3.27-3.22 (q, J=7.2 Hz, 2H), 2.56 (s, 3H), 1.38-1.35 (t, J=6.8 Hz, 3H), 1.11-1.07 (t, J=6.8 Hz, 3H).

### Amides 14b (3007), 14c (3013), and 14 k (3026)

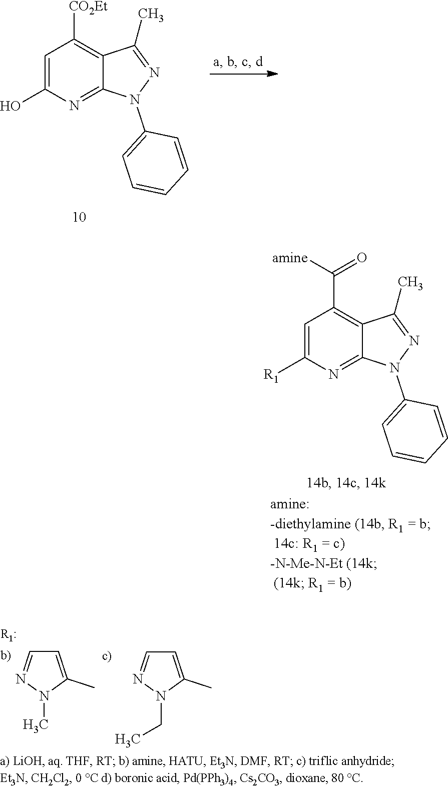

The appropriate pyrazolopyridine triflate (either ester or amide) was dissolved in dioxane. The desired boronic acid (2 eq.) and Cs₂CO₃ (3 eq.) were added to the solution respectively under N₂. After flushing with N₂ for five minutes, 10 mol % Pd (PPh₃)₄ was added, followed by N₂ flush for 5 minutes. Then reaction mixture was stirred at 80° C. for overnight. Upon completion of reaction, the reaction mixture was poured into ice cold water and extracted with three portions of ethyl acetate, washed with water, brine, dried and concentrated to afford crude product. The crude product was purified by flash chromatography eluting with hexanes/ethyl acetate.

14b (3007): 66% yield, ¹H NMR (400 MHz, CDCl₃) δ 8.20-8.18 (d, J=8.4 Hz, 2H), 7.53-7.49 (m, 3H), 7.35 (s, 1H), 7.33-7.29 (t, J=7.2 Hz, 1H), 6.71 (s, 1H), 4.33 (s, 3H), 3.69 (br, 2H), 3.28-3.22 (q, J=7.2 Hz, 2H), 2.58 (s, 3H), 1.38-1.34 (t, J=7.2 Hz, 3H), 1.13-1.09 (t, J=6.8 Hz, 3H).

14c (3013): 74% yield, ¹H NMR (400 MHz, CDCl₃) (8.22-8.20 (dd, J=8.7 Hz, 1 Hz, 2H), 7.62-7.61 (d, J=2.1 Hz, 1H), 7.58-7.54 (t, J=7.6 Hz, 2H), 7.42 (s, 1H), 7.39-7.37 (t, J 7.3 Hz, 1H), 6.77 (d, J 2 Hz, 1H), 4.85-4.83 (q, J=7 Hz, 2H), 3.84 (br, 2H), 3.32-3.30 (q, J=7 Hz, 2H), 2.64 (s, 3H), 1.57-1.54 (t, J=7 Hz, 3H), 1.44-1.40 (t, J=7 Hz, 3H), 1.18-1.15 (t, J=7 Hz, 3H).

14 k (3026): 63% yield, ¹H NMR (400 MHz, CDCl₃) δ 8.20-8.18 (d, J=8.4 Hz, 2H), 7.54-7.49 (t, J=8 Hz, 3H), 7.37 (s, 1H), 7.337.21 (t, J=7.2 Hz, 1H), 6.74-6.72 (d, J=8.4 Hz, 1H), 4.34 (s, 3H), 3.72 (br s, 1H), 3.30-3.25 (q, J=6.8 Hz, 1H), 3.21 (s, Rotamer2 CH₃), 2.90 (s Rotamer 1, CH₃), 2.57-2.56 (d, J=5.6 Hz, 3H), 1.36-1.33 (q, J=7.6 Hz, Rotamer 2, CH₃), 1.15-1.11 (q, J=7.2 Hz, Rotamer 1 CH₃).

### 23b (3030)

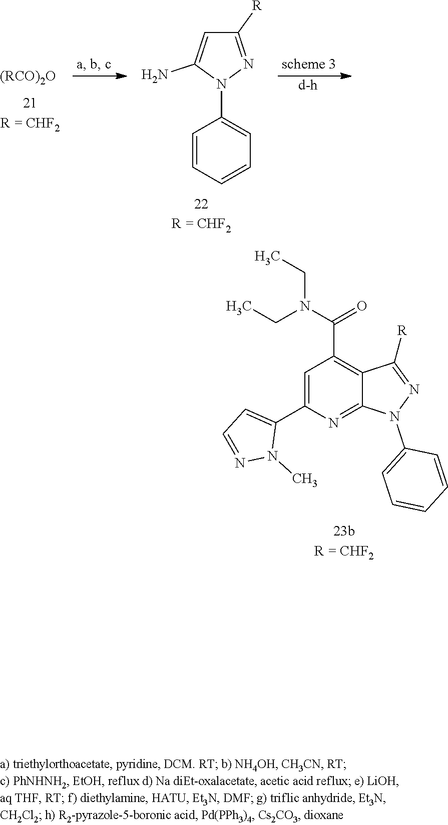

To a solution of triethyl orthoacetate (1 eq.) and pyridine (2.2 eq.) in dichloromethane, the corresponding fluoroacetic anhydride (2 eq) was added dropwise at 0° C. The reaction mixture warmed to room temperature stirred overnight at which time it was poured into cold sodium bicarbonate solution. The organic phase was washed with the water and brine, then dried and concentrated to furnish a yellow liquid that was used without further purification. NH₄OH solution (9 ml) was added to solution of this intermediate (~42 mmol) in acetonitrile and stirred for 6 hours at room temperature. The reaction was concentrated and diluted with dichloromethane. This solution was washed with water and brine then concentrated to get a yellow solid which was used for next step without purification. This crude material was dissolved in ethanol and phenyl hydrazine (1.2 eq) was added. The reaction mixture was refluxed overnight. After completion of the reaction, it was poured into ice cold water and extracted with three portions of ethyl acetate. The organic extract was washed with water, brine, dried and concentrated to furnish a solid that was used without further purification.

Using amino pyrazole 22, procedures identical to those reported earlier were used to provide target compounds. Yields reported are based on Suzuki coupling of intermediate amide-triflate.

23b (3030): 52% yield, ¹H NMR (400 MHz, CDCl₃) δ 8.20-8.18 (m, 2H), 7.61-7.56 (m, 4H), 7.46-7.42 (m, 1H), 7.19-6.92 (t, J=54 Hz), 6.83-6.82 (d, J=2.0 Hz, 1H), 4.36 (s, 3H), 3.71 (br, 2H), 3.34-3.28 (q, J=7.2 Hz, 2H), 1.40-1.37 (t, J=7.2 Hz, 3H), 1.17-1.14 (t, J=7.2 Hz, 3H).

### 25

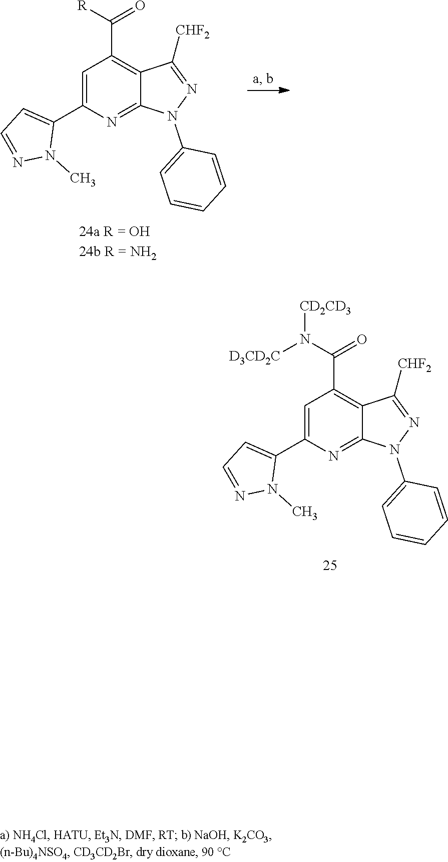

24b: To a solution of 24a (0.13 mmol, 1 eq.) in DMF was added HATU (0.15 mmol, 1.2 eq.) and NH4Cl (0.26 mmol, 1.2 eq.) respectively. Triethylamine (0.65 mmol, 5 eq.) was added drop-wise and stirred at room temperature for overnight under N₂. The reaction mixture was poured into ice water and the yellow precipitate that formed was collected by filtration, washed with water, and dried to furnish the desired amide in 78% yield.

¹H NMR (400 MHz, DMSO) δ 8.62 (s, 1H), 8.18-8.16 (m, 4H), 8.12 (s, 1H), 7.78-7.63 (m, 4H), 7.53-7.49 (m, 1H), 7.22-7.22 (d, J=2 Hz, 1H), 4.29 (s, 3H).

25: Dry dioxane (10 mL) was added to 24b (0.12 mmol, 1 eq.) under N₂. To this mixture K₂CO₃ (0.17 mmol, 1.2 eq.), Tetrabutylammonium hydrogen sulfate (TBAHS) (0.12 mmol, 1 eq.) and ground NaOH (0.5 mmol, 4 eq.) were added. The reaction mixture was heated to 40° C. and d₅-bromoethane (0.76 mmol, 6 eq.) was added dropwise. The reaction was stirred at 90° C. overnight. The reaction was quenched by adding ice and was extracted using 3×10 mL ethyl acetate. The collected organic extract was washed with water and brine. After drying and concentration, the product was purified by silica gel chromatography eluting with hexanes/ethyl acetate system in 54% yield.

¹H NMR (400 MHz, CDCl₃) δ 8.21-8.19 (m, 2H), 7.63-7.57 (m, 4H), 7.48-7.44 (m, 1H), 7.19-6.92 (t, J=54 Hz, 1H), 6.83-6.82 (d, J=2.0 Hz, 1H), 4.38 (s, 3H). M+H 435.

### PDE11A4 Enzymatic Assays

In vitro enzyme assays were conducted via the Ba(OH)₂ precipitation method of Wang et al. using recombinant human PDE3A, PDE4D3, PDE5A, PDE6C, PDE10A1, and PDE11A4 (BPS Bioscience)). Substrate concentrations used were 15 nM cGMP (PDE3A), 18 nM cAMP (PDE3A) 200 nM cAMP (PDE4D3), 500 nM cGMP (PDE5A), 1.7 μM cGMP (PDE6C), 30 nM cAMP (PDE10A), 1.3 M cGMP (PDE10A), 100 nM cGMP (PDE11A4) and 240 nM cAMP (PDE11A4). Inhibitor concentrations using a 10 point curve that reduce enzyme activity by 50% (IC₅₀) are presented as calculated using an online IC₅₀ Calculator. Inhibitor concentrations of 4, 13.7, 40, 123, 370, 1110, 3330 and 10,000 nM were used. The values reported are means of at least three independent experiments. Substrate concentrations were ~0.1×KM for each enzyme; thus, IC₅₀ values approximate the Ki values.

### Cell-Based Assay

HT-22 cells (sex undefined) were cultured and transfected as previously described.³⁰ Cells were maintained in T-75 flasks in Dulbecco's Modified Eagle Medium (DMEM) with sodium pyruvate, 1% Penicillin/Streptomycin (P/S), and 10% fetal bovine serum (FBS), with incubators set to 37° C./5% CO₂. Cells were passaged at ~70% confluency using TrypLE Express. The day before transfection, cells were plated in 60 mm dishes with DMEM+FBS+P/S. The day of transfection, the media was replaced with Optimem and cells were transfected using 5 microliters Lipofec-tamine 2000, 1.875 ug of plasmid DNA, and 5 mL optimem as per the manufacturer's protocol. ~19 hours post-transfection (PT), the Optimem/Lipofectamine solution was replaced with DMEM+FBS+P/S. Cells continued growing for five hours in the supplemented media and then were pharmacologically treated (0.01, 0.1, 1.0. 10 and 100 milli-molar) for 1 hour. After 1 hour, the media was removed, the cells were harvested in buffer (20 mM Tris-HCL and 10 mM MgCl2) and homogenized using a tissue sonicator (output control: 7.5, duty cycle: 70, continuous), and then samples were held at 4 degrees Celsius until processing. Both cAMP- and cGMP-PDE activity were measured as previously described. Samples were incubated with 35000-45000 counts per minute (CPMs) of [³H]-cAMP or [³H]-cGMP for 10 minutes. The reaction was then quenched with 0.1 M HCL and neutralized using 0.1 M Tris. Snake venom was then added to the sample and incubated for 10 minutes at 37° C. Samples were then run down DEAE A-25 sephadex columns previously equilibrated in high salt buffer (20 mM Tris-HCL, 0.1% sodium azide, and 0.5M NaCl) and low salt buffer (20 mM Tris-HCL and 0.1% sodium azide). After washing the columns four times with 0.5 ml of low salt buffer, the eluate was mixed with 4 ml of scintillation cocktail, and then CPMs were read on a Beckman-Coulter liquid scintillation counter. Two reactions not containing any sample lysate were also taken through the assay to assess background, which was subtracted from the sample CPMs. CPMs were then normalized as a function of total protein levels, which were quantified using the DC Protein Assay Kit according to the manufacturer's directions.

### Example 7: Effects of PDE11A Inhibitors in an In Vitro Model of Aging

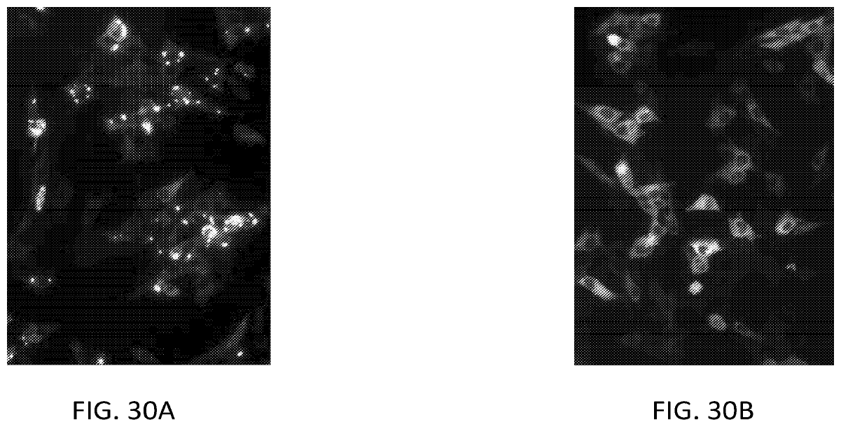

In vivo, expression of PDE11A4 increases in the hippocampus across the lifespan in humans and rodents, resulting in age-related punctate accumulations of PDE11A4 protein in “ghost axons.” These age-related PDE11A4 accumulations were mimicked by overexpressing mouse PDE11A4 in mouse HT-22 hippocampal cells. An image of HT22 cells overexpressing EmGFP-mPDE11A4 is shown in FIG. 30. FIGS. 31A-31B is an image of HT-22 cells overexpressing EmGFP-mPDE11A4 treated with the vehicle DMSO (FIG. 30A) or 1 uM 25b (FIG. 30B).

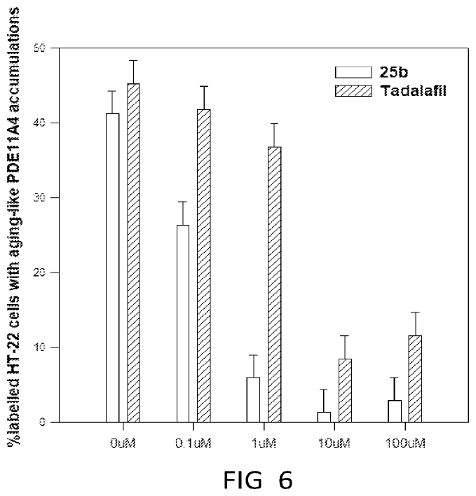

PDE11A inhibitor compounds 9a, 14 k, 14b and 25b were able to reverse these aging-like accumulations of mPDE11A4 in HT-22 cells, along with the drug Tadalafil, a PDE5 inhibitor that also potently inhibits PDE11A4, along with the commercially-available PDE11A4 inhibitor BC11-38. See FIGS. 35A and 35B, which show images of HT-22 cells overexpressing EmGFP-mPDE11A4 treated with the vehicle DMSO (35A) or 1 uM 25b (35B). Further, the compound 25b was found to be more potent and more efficacious than the drug tadalafil in reversing this aging-like PDE11A4 phenotype (FIG. 6).

### Example 8: PDE11A4 Inhibition

Compounds of the disclosure were evaluated in vitro for PDE11A4 inhibition:
Compound 1001: 62% inhibition @500 nM.
Compound 1003: IC₅₀: 140 nM.
Compound 1005: IC₅₀: 170 nM.
Compound 1007: IC₅₀: 70 nM.

### REFERENCES

1. Kelly M P. A Role for Phosphodiesterase 11A (PDE11A) in the Formation of Social Memories and the Stabilization of Mood. Advances in neurobiology. 2017; 17:201-30. doi: 10.1007/978-3-319-58811-7_8. PubMed PMID: 28956334; PubMed Central PMCID: PMC5652326.
2. Hegde S. Capell W R, Ibrahim B A, Klett J, Patel N S, Sougiannis A T, Kelly M P. Phosphodiesterase 11 A (PDE11A), Enriched in Ventral Hippocampus Neurons, is Required for Consolidation of Social but not Nonsocial Memories in Mice. Neuropsychopharmacology: official publication of the American College of Neuropsychopharmacology. 2016; 41(12):2920-31. doi: 10.1038/npp.2016.106. PubMed PMID: 27339393; PubMed Central PMCID: PMC5061884.
3. Abbott A. Cognition: The brain's decline. Nature. 2012; 492(7427):S4-5. doi: 10.1038/492S4a. PubMed PMID: 23222672.
4. Guerreiro R, Bras J. The age factor in Alzheimer's disease. Genome medicine. 2015; 7:106. doi: 10.1186/s13073-015-0232-5. PubMed PMID: 26482651; PubMed Central PMCID: PMC4617238.
5. Plassman B L, Langa K M, Fisher G G, Heeringa S G, Weir D R, Ofstedal M B, Burke J R, Hurd M D, Potter G G, Rodgers W L, Steffens D C, McArdle J J, Willis R J, Wallace R B. Prevalence of cognitive impairment without dementia in the United States. Annals of internal medicine. 2008; 148(6):427-34. PubMed PMID: 18347351; PubMed Central PMCID: PMC2670458.
6. Bender A R, Naveh-Benjamin M, Raz N. Associative deficit in recognition memory in a lifespan sample of healthy adults. Psychology and aging. 2010; 25(4):940-8. doi: 10.1037/a0020595. PubMed PMID: 20822256; PubMed Central PMCID: PMC3011045.
7. Yuasa K, Ohgaru T, Asahina M, Omori K. Identification of rat cyclic nucleotide phosphodiesterase 11 A (PDE11 A): comparison of rat and human PDE1 1 A splicing variants. European jthenal of biochemistry/FEBS. 2001; 268(16):4440-8. PubMed PMID: 11502204.
8. Yuasa K, Kanoh Y, Okumura K, Omori K. Genomic organization of the human phosphodiesterase PDE11A gene. Evolutionary relatedness with other PDEs containing GAF domains. European jthenal of biochemistry/FEBS. 2001; 268(1):168-78. PubMed PMID: 11121118.
9. Wong M L, Whelan F, Deloukas P, Whittaker P, Delgado M, Cantor R M, McCann S M, Licinio J, Wong M L, Whelan F, Deloukas P, Whittaker P, Delgado M, Cantor R M, McCann S M, Licinio J. Phosphodiesterase genes are associated with susceptibility to major depression and antidepressant treatment response. Proceedings of the National Academy of Sciences of the United States of America. 2006; 103(41):15124-9.
10. Luo H R, Wu G S, Dong C, Arcos-Burgos M, Ribeiro L, Licinio J, Wong M L. Association of PDE11A global haplotype with major depression and antidepressant drug response. Neuropsychiatric disease and treatment. 2009; 5:163-70. PubMed PMID: 19557111; PubMed Central PMCID: PMC2695232.
11. Cabanero M, Laje G, Detera-Wadleigh S, McMahon F J. Association study of phosphodiesterase genes in the Sequenced Treatment Alternatives to Relieve Depression sample. Pharmacogenetics and genomics. 2009; 19(3):235-8. doi: 10.1097/FPC.0b013e328320a3e2. PubMed PMID: 19214142; PubMed Central PMCID: PMC2819180.
12. Coon H, Darlington T, Pimentel R, Smith K R, Huff C D, Hu H, Jerominski L, Hansen J, Klein M, Callor W B, Byrd J, Bakian A, Crowell S E, McMahon W M, Rajamanickam V, Camp N J, McGlade E, Yurgelun-Todd D, Grey T, Gray D. Genetic risk factors in two Utah pedigrees at high risk for suicide. Translational psychiatry. 2013; 3:e325. doi: 10.1038/tp.2013.100. PubMed PMID: 24252905; PubMed Central PMCID: PMC3849959.
13. Perlis R H, Fijal B, Dharia S, Heinloth A N, Houston J P. Failure to replicate genetic associations with antidepressant treatment response in duloxetine-treated patients. Biological psychiatry. 2010; 67(11):1110-3. doi: 10.1016/j.biopsych.2009.12.010. PubMed PMID: 20110084.
14. Couzin J. Science and commerce. Gene tests for psychiatric risk polarize researchers. Science. 2008; 319(5861):274-7. doi: 10.1126/science.319.5861.274. PubMed PMID: 18202268.
15. Kelsoe J, inventor; THE REGENTS OF THE UNIVERSITY OF CALIFORNIA (Oakland, C A), assignee. METHOD TO PREDICT RESPONSE TO TREATMENT FOR PSYCHIATRIC ILLNESSES. USA2010.
16. Mertens J, Wang Q W, Kim Y, Yu D X, Pham S, Yang B, Zheng Y, Diffenderfer K E, Zhang J, Soltani S, Eames T, Schafer S T, Boyer L, Marchetto M C, Nurnberger J I, Calabrese J R, Odegaard K J, McCarthy M J, Zandi P P, Alda M, Nievergelt C M, Pharmacogenomics of Bipolar Disorder S, Mi S, Brennand K J, Kelsoe J R, Gage F H, Yao J. Differential responses to lithium in hyperexcitable neurons from patients with bipolar disorder. Nature. 2015; 527(7576):95-9. doi: 10.1038/nature15526. PubMed PMID: 26524527; PubMed Central PMCID: PMC4742055.
17. Rizzo L B, Costa L G, Mansur R B, Swardfager W, Belangero S I, Grassi-Oliveira R, McIntyre R S, Bauer M E, Brietzke E. The theory of bipolar disorder as an illness of accelerated aging: implications for clinical care and research. Neuroscience and biobehavioral reviews. 2014; 42:157-69. doi: 10.1016/j.neubiorev.2014.02.004. PubMed PMID: 24548785.
18. Kinser P A, Lyon D E. Major depressive disorder and measures of cellular aging: an integrative review. Nursing research and practice. 2013; 2013:469070. doi: 10.1155/2013/469070. PubMed PMID: 23691300; PubMed Central PMCID: PMC3649747.
19. Kelly M P, Logue S F, Brennan J, Day J P, Lakkaraju S, Jiang L, Zhong X, Tam M, Sukoff Rizzo S J, Platt B J, Dwyer J M, Neal S, Pulito V L, Agostino M J, Grauer S M, Navarra R L, Kelley C, Comery T A, Murrills R J, Houslay M D, Brandon N J. Phosphodiesterase 11A in brain is enriched in ventral hippocampus and deletion causes psychiatric disease-related phenotypes. Proceedings of the National Academy of Sciences of the United States of America. 2010; 107(18):8457-62. doi: 10.1073/pnas.1000730107. PubMed PMID: 20404172; PubMed Central PMCID: PMC2889541.
20. Kelly M P. Does phosphodiesterase 11A (PDE11A) hold promise as a future therapeutic target?Current pharmaceutical design. 2015; 21(3):389-416. PubMed PMID: 25159071.
21. Kelly M P. A Role for Phosphodiesterase 11A (PDE11A) in the Formation of Social Memories and the Stabilization of Mood. Advances in neurobiology. 2017; 17:201-30. doi: 10.1007/978-3-319-58811-7_8. PubMed PMID: 28956334; PubMed Central PMCID: PMC5652326.
22. Hegde S. Capell W R, Ibrahim B A, Klett J, Patel N S, Sougiannis A T, Kelly M P. Phosphodiesterase 11 A (PDE11A), Enriched in Ventral Hippocampus Neurons, is Required for Consolidation of Social but not Nonsocial Memories in Mice. Neuropsy chopharmacology: official publication of the American College of Neuropsychopharmacology. 2016; 41(12):2920-31. doi: 10.1038/npp.2016.106. PubMed PMID: 27339393; PubMed Central PMCID: PMC5061884.
23. Pathak G, Agostino M J, Bishara K₂, Capell W R, Fisher J L, Hegde S, Ibrahim B A, Pilarzyk K, Sabin C, Tuczkewycz T, Wilson S, Kelly M P. PDE11A negatively regulates lithium responsivity. Molecular psychiatry. 2017; 22(12):1714-24. doi: 10.1038/mp.2016.155. PubMed PMID: 27646265; PubMed Central PMCID: PMC5359083.
24. Abbott A. Cognition: The brain's decline. Nature. 2012; 492(7427):54-5. doi: 10.1038/492S4a. PubMed PMID: 23222672.
25. Terry R, Cheung Y F, Praestegaard M. Baillie G S, Huston E, Gall I, Adams D R, Houslay M D. Occupancy of the catalytic site of the PDE4A4 cyclic AMP phosphodiesterase by rolipram triggers the dynamic redistribution of this specific isoform in living cells through a cyclic AMP independent process. Cellular signalling. 2003; 15(10):955-71. PubMed PMID: 12873709.
26. Guerreiro R, Bras J. The age factor in Alzheimer's disease. Genome medicine. 2015; 7:106. doi: 10.1186/s13073-015-0232-5. PubMed PMID: 26482651; PubMed Central PMCID: PMC4617238.
27. Plassman B L, Langa K M, Fisher G G, Heeringa S G, Weir D R, Ofstedal M B, Burke J R, Hurd M D, Potter G G, Rodgers W L, Steffens D C, McArdle J J, Willis R J, Wallace R B. Prevalence of cognitive impairment without dementia in the United States. Annals of internal medicine. 2008; 148(6):427-34. PubMed PMID: 18347351; PubMed Central PMCID: PMC2670458.
28. Bender A R, Naveh-Benjamin M, Raz N. Associative deficit in recognition memory in a lifespan sample of healthy adults. Psychology and aging. 2010; 25(4):940-8. doi: 10.1037/a0020595. PubMed PMID: 20822256; PubMed Central PMCID: PMC3011045.
29. Old S R, Naveh-Benjamin M. Differential effects of age on item and associative measures of memory: a meta-analysis. Psychology and aging. 2008; 23(1):104-18. doi: 10.1037/0882-7974.23.1.104. PubMed PMID: 18361660.
30. Bridger E K, Kursawe A L, Bader R, Tibon R, Gronau N, Levy D A, Mecklinger A. Age effects on associative memory for novel picture pairings. Brain research. 2017; 1664:102-15. doi: 10.1016/j.brainres.2017.03.031. PubMed PMID: 28377157.
31. Troyer A K, D'Souza N A, Vandermorris S, Murphy K J. Age-related differences in associative memory depend on the types of associations that are formed. Neuropsychology, development, and cognition Section B, Aging, neuropsychology and cognition. 2011; 18(3):340-52. doi: 10.1080/13825585.2011.553273. PubMed PMID: 21390876.
32. Overman A A, Becker J T. The associative deficit in older adult memory: Recognition of pairs is not improved by repetition. Psychology and aging. 2009; 24(2):501-6. doi: 10.1037/a0015086. PubMed PMID: 19485666; PubMed Central PMCID: PMC2752499.
33. Hartman M, Warren L H. Explaining age differences in temporal working memory. Psychology and aging. 2005; 20(4):645-56. doi: 10.1037/0882-7974.20.4.645. PubMed PMID: 16420139.
34. Hargis M B, Castel A D. Younger and older adults' associative memory for social information: The role of information importance. Psychology and aging. 2017; 32(4):325-30. doi: 10.1037/pag0000171. PubMed PMID: 28581330; PubMed Central PMCID: PMC5499679.
35. Nordin K, Herlitz A, Larsson E M, Soderlund H. Overlapping effects of age on associative memory and the anterior hippocampus from middle to older age. Behavitheal brain research. 2017; 317:350-9. doi: 10.1016/j.bbr.2016.10.002. PubMed PMID: 27713000.
36. Dalton M A, Tu S, Homberger M, Hodges J R, Piguet O. Medial temporal lobe contributions to intra-item associative recognition memory in the aging brain. Frontiers in behavioral neuroscience. 2013; 7:222. doi: 10.3389/fnbeh.2013.00222. PubMed PMID: 24427127; PubMed Central PMCID: PMC3878719.
37. Yuasa K, Ohgaru T, Asahina M, Omori K. Identification of rat cyclic nucleotide phosphodiesterase 11A (PDE11A): comparison of rat and human PDE11A splicing variants. European jthenal of biochemistry/FEBS. 2001:268(16):4440-8. PubMed PMID: 11502204.
38. Yuasa K, Kanoh Y, Okumura K, Omori K. Genomic organization of the human phosphodiesterase PDE11A gene. Evolutionary relatedness with other PDEs containing GAF domains. European jthenal of biochemistry/FEBS. 2001; 268(1):168-78. PubMed PMID: 11121118.
39. Wong M L, Whelan F, Deloukas P, Whittaker P, Delgado M. Cantor R M, McCann S M, Licinio J, Wong M L, Whelan F, Deloukas P, Whittaker P, Delgado M, Cantor R M, McCann S M, Licinio J. Phosphodiesterase genes are associated with susceptibility to major depression and antidepressant treatment response. Proceedings of the National Academy of Sciences of the United States of America. 2006; 103(41):15124-9.
40. Luo H R, Wu G S, Dong C, Arcos-Burgos M, Ribeiro L, Licinio J, Wong M L. Association of PDE11A global haplotype with major depression and antidepressant drug response. Neuropsychiatric disease and treatment. 2009; 5:163-70. PubMed PMID: 19557111; PubMed Central PMCID: PMC2695232.
41. Cabanero M, Laje G, Detera-Wadleigh S, McMahon F J. Association study of phosphodiesterase genes in the Sequenced Treatment Alternatives to Relieve Depression sample. Pharmacogenetics and genomics. 2009; 19(3):235-8. doi: 10.1097/FPC.0b013e328320a3e2. PubMed PMID: 19214142; PubMed Central PMCID: PMC2819180.
42. Coon H, Darlington T, Pimentel R, Smith K R, Huff C D, Hu H, Jerominski L, Hansen J, Klein M, Callor W B, Byrd J, Bakian A, Crowell S E, McMahon W M, Rajamanickam V, Camp N J, McGlade E, Yurgelun-Todd D, Grey T, Gray D. Genetic risk factors in two Utah pedigrees at high risk for suicide. Translational psychiatry. 2013; 3:e325. doi: 10.1038/tp.2013.100. PubMed PMID: 24252905; PubMed Central PMCID: PMC3849959.
43. Couzin J. Science and commerce. Gene tests for psychiatric risk polarize researchers. Science. 2008; 319(5861):274-7. doi: 10.1126/science.319.5861.274. PubMed PMID: 18202268.
44. Kelsoe J, inventor; THE REGENTS OF THE UNIVERSITY OF CALIFORNIA (Oakland, C A), assignee. METHOD TO PREDICT RESPONSE TO TREATMENT FOR PSYCHIATRIC ILLNESSES. USA2010.
45. Mertens J, Wang Q W, Kim Y, Yu D X, Pham S, Yang B, Zheng Y, Diffenderfer K E, Zhang J, Soltani S, Eames T, Schafer S T, Boyer L, Marchetto M C, Numberger J I, Calabrese J R, Odegaard K J, McCarthy M J, Zandi P P, Alda M, Nievergelt C M, Pharmacogenomics of Bipolar Disorder S, Mi S, Brennand K J, Kelsoe J R, Gage F H, Yao J. Differential responses to lithium in hyperexcitable neurons from patients with bipolar disorder. Nature. 2015; 527(7576):95-9. doi: 10.1038/naturel5526. PubMed PMID: 26524527; PubMed Central PMCID: PMC4742055.
46. Rizzo L B, Costa L G, Mansur R B, Swardfager W, Belangero S I, Grassi-Oliveira R, McIntyre R S, Bauer M E, Brietzke E. The theory of bipolar disorder as an illness of accelerated aging: implications for clinical care and research. Neuroscience and biobehavioral reviews. 2014; 42:157-69. doi: 10.1016/j.neubiorev.2014.02.004. PubMed PMID: 24548785.
47. Kinser P A, Lyon D E. Major depressive disorder and measures of cellular aging: an integrative review. Nursing research and practice. 2013; 2013:469070. doi: 10.1155/2013/469070. PubMed PMID: 23691300; PubMed Central PMCID: PMC3649747.
48. Kelly M P, Logue S F, Brennan J, Day J P, Lakkaraju S, Jiang L, Zhong X, Tam M, Sukoff Rizzo S J, Platt B J, Dwyer J M, Neal S, Pulito V L, Agostino M J, Grauer S M, Navarra R L, Kelley C, Comery T A, Murrills R J, Houslay M D, Brandon N J. Phosphodiesterase 11A in brain is enriched in ventral hippocampus and deletion causes psychiatric disease-related phenotypes. Proceedings of the National Academy of Sciences of the United States of America. 2010; 107(18):8457-62. doi: 10.1073/pnas.1000730107. PubMed PMID: 20404172; PubMed Central PMCID: PMC2889541.
49. Kelly M P. Does phosphodiesterase 11A (PDE11A) hold promise as a future therapeutic target?Current pharmaceutical design. 2015; 21(3):389-416. PubMed PMID: 25159071.
50. Kelly M P. PDE11A. In: Choi S, editor. Encyclopedia of Signaling Molecules. Chain: Springer International Publishing; 2018, p. 3804-26.
51. Kelly M P, Adamowicz W, Bove S, Hartman A J, Mariga A, Pathak G, Reinhart V, Romegialli A, Kleiman R J. Select 3′,5′-cyclic nucleotide phosphodiesterases exhibit altered expression in the aged rodent brain. Cellular signalling. 2014; 26(2):383-97. doi: 10.1016/j.cellsig.2013.10.007. PubMed PMID: 24184653.
52. Jager R, Russwurm C, Schwede F, Genieser H G, Koesling D, Russwurm M. Activation of PDE10 and PDE11 phosphodiesterases. The Jthenal of biological chemistry. 2012; 287(2):1210-9. doi: 10.1074/jbc.M111.263806. PubMed PMID: 22105073; PubMed Central PMCID: PMC3256904.
53. Pilarzyk K. Klett J, Pena E A, Porcher L, Smith A J, Kelly M P. Loss of Function of Phosphodiesterase 11A4 Shows that Recent and Remote Long-Term Memories Is Uncoupled. Current biology: C B. 2019. doi: 10.1016/j.cub.2019.06.018. PubMed PMID: 31303492.
54. Hegde S, Ji H, Oliver D, Patel N S, Poupore N, Shtutman M, Kelly M P. PDE11A regulates social behaviors and is a key mechanism by which social experience sculpts the brain. Neuroscience. 2016; 335:151-69. doi: 10.1016/j.neuroscience.2016.08.019. PubMed PMID: 27544407; PubMed Central PMCID: PMC5031549.
55. Zhang C, Cheng Y. Wang H, Wang C, Wilson S P, Xu J, Zhang H T. RNA interference-mediated knockdown of long-form phosphodiesterase-4D (PDE4D) enzyme reverses amyloid-beta42-induced memory deficits in mice. Jthenal of Alzheimer's disease: JAD. 2014; 38(2):269-80. doi: 10.3233/JAD-122236. PubMed PMID: 23948935.
56. Bonkale W L, Cowbum R F, Ohm T G, Bogdanovic N, Fastbom J. A quantitative autoradiographic study of [3H]cAMP binding to cytosolic and particulate protein kinase A in post-mortem brain staged for Alzheimer's disease neurofibrillary changes and amyloid deposits. Brain research. 1999; 818(2):383-96. PubMed PMID: 10082824.
57. Titus D J, Furones C, Kang Y, Atkins C M. Age-dependent alterations in cAMP signaling contribute to synaptic plasticity deficits following traumatic brain injury. Neuroscience. 2013; 231:182-94. doi: 10.1016/j.neuroscience.2012.12.002. PubMed PMID: 23238576; PubMed Central PMCID: PMC3558555.
58. Ceyhan O, Birsoy K, Hoffman C S. Identification of Biologically Active PDE11-Selective Inhibitors Using a Yeast-Based High-Throughput Screen. Chem Biol. 2012; 19(1):155-63. Epub 2012/01/31. doi: 10.1016/j.chembiol.2011.12.010. PubMed PMID: 22284362.
59. Countryman R A, Gold P E. Rapid forgetting of social transmission of food preferences in aged rats: relationship to hippocampal CREB activation. Learning & memory. 2007; 14(5):350-8. doi: 10.1101/lm.524907. PubMed PMID: 17522026; PubMed Central PMCID: PMC1876759.
60. Patel N, Klett J, Pilarzyk K, Lee D I, Kass D A, Menniti F, Kelly M P. Identification of new phosphodiesterase 9A (PDE9A) isoforms and how their expression and subcellular compartmentalization in brain changes across the lifespan. Neurobiology of aging. in press.
61. Alaamery M A, Wyman A R, Ivey F D, Allain C, Demirbas D, Wang L, Ceyhan O, Hoffman C S. New classes of PDE7 inhibitors identified by a fission yeast-based HTS. J Biomol Screen. 2010; 15(4):359-67. Epub 2010/03/17. doi: 10.1177/1087057110362100. PubMed PMID: 20228279; PubMed Central PMCID: PMC2854023.
62. de Medeiros A S, Wyman A R, Alaamery M A, Allain C, Ivey F D, Wang L. Le H, Morken J P, Habara A, Le C, Cui S, Lerner A, Hoffman C S. Identification and characterization of a potent and biologically-active PDE4/7 inhibitor via fission yeast-based assays. Cellular signalling. 2017; 40:73-80. doi: 10.1016/j.cellsig.2017.08.011. PubMed PMID: 28867658; PubMed Central PMCID: PMC5651194.
63. Demirbas D, Wyman A R, Shimizu-Albergine M, Cakici O, Beavo J A, Hoffman C S. A Yeast-Based Chemical Screen Identifies a PDE Inhibitor that Elevates Steroidogenesis in Mouse Leydig Cells Via PDE8 and PDE4 Inhibition. PLoS One. 2013; 8(8):e71279. doi: 10.1371/jthenal.pone.0071279.
64. Ivey F D, Wang L, Demirbas D, Allain C, Hoffman C S. Development of a fission yeast-based high-throughput screen to identify chemical regulators of cAMP phosphodiesterases. J Biomol Screen. 2008; 13(1):6271. PubMed PMID: 18227226.
65. Ceyhan. Identification of biologically-active PDE11-selective inhibitors using a yeast-based high throughput screen. eScholarship@B C: Boston College; 2012.
66. Smith S J, Cieslinski L B, Newton R, Donnelly L E, Fenwick P S, Nicholson A G, Barnes P J, Bamette M S, Giembycz M A. Discovery of BRL 50481 [3—(N,N-dimethylsulfonamido)-4-methyl-nitrobenzene], a selective inhibitor of phosphodiesterase 7: in vitro studies in human monocytes, lung macrophages, and CD8+T-lymphocytes. Mol Pharmacol. 2004; 66(6):1679-89. PubMed PMID: 15371556.
67. Demirbas D, Ceyhan 0, Wyman A R, Ivey F D, Allain C, Wang L, Sharuk M N, Francis S H, Hoffman C S. Use of a Schizosaccharomyces pombe PKA-repressible reporter to study cGMP metabolising phosphodiesterases. Cell Signal. 2011; 23(3):594-601. Epub 2010/12/02. doi: 10.1016/j.cellsig.2010.11.013. PubMed PMID: 21118717; PubMed Central PMCID: PMC3012454.
68. Demirbas D, Ceyhan 0, Wyman A R, Hoffman C S. A fission yeast-based platform for phosphodiesterase inhibitor HTSs and analyses of phosphodiesterase activity. Handb Exp Pharmacol. 2011(204):135-49. Epub 2011/06/23. doi: 10.1007/978-3-642-17969-3_5. PubMed PMID: 21695638.
69. Denny P W, Steel P G. Yeast as a potential vehicle for neglected tropical disease drug discovery. J Biomol Screen. 2015; 20(1):56-63. doi: 10.1177/1087057114546552. PubMed PMID: 25121554.
70. Denny P W. Yeast: bridging the gap between phenotypic and biochemical assays for high-throughput screening. Expert Opin Drug Discov. 2018:1-8. doi: 10.1080/17460441.2018.1534826. PubMed PMID: 30326751.
71. Kim D U, Hayles J, Kim D, Wood V, Park H O, Won M, Yoo H S, Duhig T, Nam M, Palmer G, Han S, Jeffery L, Baek S T, Lee H, Shim Y S, Lee M, Kim L, Heo K S, Noh E J, Lee A R, Jang Y J, Chung K S, Choi S J, Park J Y, Park Y, Kim H M, Park S K, Park H J, Kang E J, Kim H B, Kang H S, Park H M, Kim K, Song K, Song K B, Nurse P, Hoe K L. Analysis of a genome-wide set of gene deletions in the fission yeast Schizosaccharomyces pombe. Nat Biotechnol. 2010; 28(6):617-23. doi: 10.1038/nbt.1628. PubMed PMID: 20473289; PubMed Central PMCID: PMC3962850.
72. Wager T T, Hou X, Verhoest P R, Villalobos A. Central Nervous System Multiparameter Optimization Desirability: Application in Drug Discovery. ACS chemical neuroscience. 2016; 7(6):767-75. doi: 10.1021/acschemneuro.6b00029. PubMed PMID: 26991242.
73. Hazarkhani H, Karimi B. A facile synthesis of new 3-(2-benzimidazolyl)-2-alkyl-4-(3H)quinazolinones under microwave irradiation. Tetrahedron. 2003; 59(26):4757-60. doi: 10.1016/S0040-4020(03)00696-3. PubMed PMID: WOS:000183780900006.
74. Gineinah MMM. 3-heteroaryl 3,4-dihydro-4-oxo-1,2,3-benzotriazines: Synthesis, characterization, and antibacterial evaluation. Med Chem Res. 2001; 10(9):596-604. PubMed PMID: WOS:000172050200004.
75. Qian W Y, Chen J J, Human J, Aya T, Zhu J W, Biswas K, Peterkin T, Hungate R W, Arik L, Johnson E, Kumar G, Joseph S, Jona J, Guo H X, Wu Z F. Discovery of dehydro-oxopiperazine acetamides as novel bradykinin B1 receptor antagonists with enhanced in vitro potency. Bioorganic & Medicinal Chemistry Letters. 2012; 22(2):1061-7. doi: 10.1016/j.bmcl.2011.11.112. PubMedPMID: WOS:000299653500059.
76. Volochnyuk D M, Ryabukhin S V, Plaskon A S, Dmytriv Y V, Grygorenko 00, Mykhailiuk P K, Krotko D G, Pushechnikov A, Tolmachev A A. Approach to the Library of Fused Pyridine-4-carboxylic Acids by Combes-Type Reaction of Acyl Pyruvates and Electron-Rich Amino Heterocycles. J Comb Chem. 2010; 12(4):510-7. doi: 10.1021/cc100040q. PubMed PMID: WOS:000279618200019.
77. Dang N L, Hughes T B, Miller G P, Swamidass S J. Computational Approach to Structural Alerts: Furans, Phenols, Nitroaromatics, and Thiophenes. Chem Res Toxicol. 2017; 30(4):1046-59. doi: 10.1021/acs.chemrestox.6b00336. PubMed PMID: 28256829; PubMed Central PMCID: PMCPMC5871347.
78. Jaladanki C K, Taxak N, Varikoti R A, Bharatam P V. Toxicity Originating from Thiophene Containing Drugs: Exploring the Mechanism using Quantum Chemical Methods. Chem Res Toxicol. 2015; 28(12):2364-76. doi: 10.1021/acs.chemrestox.5b00364. PubMed PMID: WOS:000367118000014.
79. Sun L, Cui J, Liang C, ZhouY, Nematalla A, Wang X, Chen H, Tang C, Wei J. Rational design of 4,5-disubstituted-5,7-dihydro-pyrrolo[2,3-d]pyrimidin-6-ones as a novel class of inhibitors of epidermal growth factor receptor (EGF-R) and Her2(p185(erbB)) tyrosine kinases. Bioorg Med Chem Lett. 2002; 12(16):2153-7. PubMed PMID: 12127526.
80. Wang H, Liu Y, Chen Y, Robinson H, Ke H. Multiple elements jointly determine inhibitor selectivity of cyclic nucleotide phosphodiesterases 4 and 7. J Biol Chem. 2005; 280(35):30949-55. PubMed PMID: 15994308.
81. Alaamery M A, Hoffman C S. Schizosaccharomyces pombe Hsp90/Git10 is required for glucose/cAMP signaling. Genetics. 2008; 178(4):1927-36. PubMed PMID: 18430926.
82. Byrne S M, Hoffman C S. Six git genes encode a glucose-induced adenylate cyclase activation pathway in the fission yeast Schizosaccharomyces pombe. J Cell Sci. 1993; 105(Pt 4):1095-100.
83. Hoffman C S, Winston F. Glucose repression of transcription of the Schizosaccharomyces pombe fbp1 gene occurs by a cAMP signaling pathway. Genes Dev. 1991; 5(4):561-71.
84. Ivey F D, Hoffman C S. Direct activation of fission yeast adenylate cyclase by the Gpa2 Ga of the glucose signaling pathway. Proc Natl Acad Sci USA. 2005; 102(17):6108-13. PubMed PMID: 15831585.
85. Ivey F D, Taglia F X, Yang F, Lander M M, Kelly D A, Hoffman C S. Activated alleles of the chizosaccharomyces pombe gpa2+ Galpha gene identify residues involved in GDP-GTP exchange. Eukaryot Cell. 2010; 9(4):626-33. Epub 2010/02/09. doi: 10.1128/E C.00010-10. PubMed PMID: 20139237; PubMed Central PMCID: PMC2863420.
86. Kao R S, Morreale E, Wang L, Ivey F D, Hoffman C S. Schizosaccharomyces pombe Git1 is a C₂-domain protein required for glucose activation of adenylate cyclase. Genetics. 2006,173(1):49-61. PubMed PMID: 16489217.
87. Landry S, Hoffman C S. The git5 Gβ and git11 Gγ Form an Atypical Goy Dimer Acting in the Fission Yeast Glucose/cAMP Pathway. Genetics. 2001; 157(3):1159-68.
88. Landry S, Pettit M T, Apolinario E, Hoffman C S. The fission yeast git5 gene encodes a G P subunit required for glucose-triggered adenylate cyclase activation. Genetics. 2000; 154(4):1463-71.
89. M A A, A R W. F D I, C A, D D, L W, O C, C S H. New classes of PDE7 inhibitors identified by a fission yeast-based HTS J Biomol Screening. 2010:in press.
90. Nocero M, Isshiki T, Yamamoto M, Hoffman C S. Glucose repression of fbp1 transcription of Schizosaccharomyces pombe is partially regulated by adenylate cyclase activation by a G protein a subunit encoded by gpa2 (git8). Genetics. 1994; 138(1):39-45.
91. Schadick K, Fourcade H M, Boumenot P, Seitz J J, Morrell J L, Chang L, Gould K L, Partridge J F, Allshire R C, Kitagawa K, Hieter P, Hoffman C S. Schizosaccharomyces pombe Git7p, a member of the Saccharomyces cerevisiae Sgtlp family, is required for glucose and cyclic AMP signaling, cell wall integrity, and septation. Eukaryot Cell. 2002; 1(4):558-67. PubMed PMID: 12456004.
92. Stiefel J, Wang L, Kelly D A, Janoo R T K, Seitz J, Whitehall S K, Hoffman C S. Suppressors of an adenylate cyclase deletion in the fission yeast Schizosaccharomyces pombe. Eukaryot Cell. 2004; 3(3):610-9.
93. Wang L, Griffiths K, Zhang Y H, Ivey F D, Hoffman C S. Schizosaccharomyces pombe adenylate cyclase suppressor mutations suggest a role for cAMP phosphodiesterase regulation in feedback control of glucose/cAMP signaling. Genetics. 2005; 171:1523-33. PubMed PMID: 16143612.
94. Welton R M, Hoffman C S. Glucose monitoring in fission yeast via the gpa2 Gα, the git5 Gβ, and the git3 putative glucose receptor. Genetics. 2000; 156:513-21.
95. Getz R A, Kwak G, Cornell S, Mbugua S, Eberhard J, Huang S X, Abbasi Z, de Medeiros A S, Thomas R, Bukowski B, Dranchak P K, Inglese J, Hoffman C S. A fission yeast platform for heterologous expression of mammalian adenylyl cyclases and high throughput screening. Cell Signal. 2019; 60:114-21. doi: 10.1016/j.cellsig.2019.04.010. PubMed PMID: 31026495.
96. Ceyhan O. Identification of biologically-active PDE11-selective inhibitors using a yeast-based high throughput screen. eScholarship@B C: Boston College; 2012.
97. Lounsbury N, Mateo G, Jones B, Papaiahgari S, Thimmulappa R K, Teijaro C, Gordon J, Korzekwa K, Ye M, Allaway G, Abou-Gharbia M, Biswal S, Childers W, Jr. Heterocyclic chalcone activators of nuclear factor (erythroid-derived 2)-like 2 (Nrf2) with improved in vivo efficacy. Bioorg Med Chem. 2015; 23(17):5352-9. doi: 10.1016/j.bmc.2015.07.056. PubMed PMID: 26278028.
98. McMasters D R, Torres R A, Crathem S J, Dooney D L, Nachbar R B, Sheridan R P, Korzekwa K R. Inhibition of recombinant cytochrome P450 isoforms 2D6 and 2C₉ by diverse drug-like molecules. Jthenal of medicinal chemistry. 2007; 50(14):3205-13. doi: 10.1021/jm0700060. PubMed PMID: 17559204; PubMed Central PMCID: PMC2547349.
99. Huang X P, Mangano T, Hufeisen S, Setola V, Roth B L. Identification of human Ether-a-go-go related gene modulators by three screening platforms in an academic drug-discovery setting. Assay Drug Dev Technol. 2010; 8(6):727-42. doi: 10.1089/adt.2010.0331. PubMed PMID: 21158687; PubMed Central PMCID: PMCPMC3002179.
100. Titus S A, Beacham D, Shahane S A, Southall N, Xia M, Huang R, Hooten E, Zhao Y, Shou L, Austin C P, Zheng W. A new homogeneous high-throughput screening assay for profiling compound activity on the human ether-a-go-go-related gene channel. Anal Biochem. 2009; 394(1):30-8. doi: 10.1016/j.ab.2009.07.003. PubMed PMID: 19583963; PubMed Central PMCID: PMCPMC2766802.
101. Grauer S M, Pulito V L, Navarra R L, Kelly M P, Kelley C, Graf R, Langen B, Logue S, Brennan J, Jiang L, Charych E, Egerland U, Liu F, Marquis K L, Malamas M, Hage T, Comery T A, Brandon N J. Phosphodiesterase 10A inhibitor activity in preclinical models of the positive, cognitive, and negative symptoms of schizophrenia. The Jthenal of pharmacology and experimental therapeutics. 2009; 331(2):574-90. Epub 2009/08/08. doi: 10.1124/jpet.109.155994. PubMed PMID: 19661377.
102. Sha L, MacIntyre L, Machell J A, Kelly M P, Porteous D J, Brandon N J, Muir W J, Blackwood D H, Watson D G, Clapcote S J, Pickard B S. Transcriptional regulation of neurodevelopmental and metabolic pathways by NPAS3. Molecular psychiatry. 2012; 17(3):267-79. Epub 2011/06/29. doi: 10.1038/mp.2011.73. PubMed PMID: 21709683.
103. Maxwell C R, Liang Y, Kelly M P, Kanes S J, Abel T, Siegel S J. Mice expressing constitutively active Gsalpha exhibit stimulus encoding deficits similar to those observed in schizophrenia patients2006; 141(3):1257-64.
104. Kelly M P, Stein J M, Vecsey C G, Favilla C, Yang X, Bizily S F, Esposito M F, Wand G, Kanes S J, Abel T. Developmental etiology for neuroanatomical and cognitive deficits in mice overexpressing Galphas, a G-protein subunit genetically linked to schizophrenia. Molecular psychiatry. 2009; 14(4):398-415, 347. Epub 2008/11/26. doi: 10.1038/mp.2008.124. PubMed PMID: 19030002; PubMed Central PMCID: PMC3312743.
105. Kelly M P, Isiegas C, Cheung Y F, Tokarczyk J, Yang X, Esposito M F, Rapoport D A, Fabian S A, Siegel S J, Wand G, Houslay M D, Kanes S J, Abel T. Constitutive activation of Galphas within forebrain neurons causes deficits in sensorimotor gating because of PKA-dependent decreases in cAMP. Neuropsychopharmacology: official publication of the American College of Neuropsychopharmacology. 2007; 32(3):577-88. doi: 10.1038/sj.npp.1301099. PubMed PMID: 16738544; PubMed Central PMCID: PMC3303872.
106. Kelly M P, Cheung Y F, Favilla C, Siegel S J, Kanes S J, Houslay M D, Abel T. Constitutive activation of the G-protein subunit Galphas within forebrain neurons causes PKA-dependent alterations in fear conditioning and cortical Arc mRNA expression. Learning & memory. 2008; 15(2):75-83. doi: 10.1101/lm.723708. PubMed PMID: 18230676; PubMed Central PMCID: PMC2216679.
107. Gould T J, Bizily S P, Tokarczyk J, Kelly M P, Siegel S J, Kanes S J, Abel T. Sensorimotor gating deficits in transgenic mice expressing a constitutively active form of Gs alpha. Neuropsychopharmacology: official publication of the American College of Neuropsychopharmacology. 2004; 29(3):494-501. Epub 2003/12/25. doi: 10.1038/sj.npp.1300309. PubMed PMID: 14694347; PubMed Central PMCID: PMC3348581.
108. Favilla C, Abel T, Kelly M P. Chronic Galphas signaling in the striatum increases anxiety-related behaviors independent of developmental effects. The Jthenal of neuroscience: the official jthenal of the Society for Neuroscience. 2008; 28(51):13952-6. Epub 2008/12/19. doi: 10.1523/JNEUROSCI.4986-08.2008. PubMed PMID: 19091983; PubMed Central PMCID: PMC2688724.
109. Bthetchouladze R. Patterson S L, Kelly M P, Kreibich A, Kandel E R, Abel T. Chronically increased Gsalpha signaling disrupts associative and spatial learning. Learning & memory. 2006; 13(6):745-52. Epub 2006/12/05. doi: 10.1101/lm.354106. PubMed PMID: 17142304; PubMed Central PMCID: PMC1783628.
110. Rizzo S J, Edgerton J R, Hughes Z A, Brandon N J. Future viable models of psychiatry drug discovery in pharma. Jthenal of biomolecular screening. 2013; 18(5):509-21. doi: 10.1177/1087057113475871. PubMed PMID: 23392517.
111. Sukoff Rizzo S J, Silverman J L. Methodological Considerations for Optimizing and Validating Behavioral Assays. Current protocols in mouse biology. 2016; 6(4):364-79. doi: 10.1002/cpmo.17. PubMed PMID: 27906464; PubMed Central PMCID: PMC6054129.
112. Garrido Zinn C, Buhler L, Cavalcante L E, Schmidt S D, Fagundes Ferreira F, Zanini M L, Guerino Furini C R, de Carvalho Myskiw J, Izquierdo I. Methylphenidate induces state-dependency of social recognition learning: Central components. Neurobiology of learning and memory. 2018; 149:77-83. doi: 10.1016/j.nlm.2018.02.002. PubMed PMID: 29408055.
113. Gonzales C, Zaleska M M, Riddell D R. Atchison K P, Robshaw A, Zhou H, Sukoff Rizzo S J. Alternative method of oral administration by peanut butter pellet formulation results in target engagement of BACE1 and attenuation of gavage-induced stress responses in mice. Pharmacology, biochemistry, and behavior. 2014; 126:2835. doi: 10.1016/j.pbb.2014.08.010. PubMed PMID: 25242810.
114. Cope M B, Nagy T R, Fernandez J R, Geary N, Casey D E, Allison D B. Antipsychotic drug-induced weight gain: development of an animal model. International jthenal of obesity. 2005; 29(6):607-14. doi: 10.1038/sj.ijo.0802928. PubMed PMID: 15795750.
115. Mooradian A D. Effect of aging on the blood-brain barrier. Neurobiology of aging. 1988; 9(1):31-9. PubMed PMID: 3288893.
116. Sukoff Rizzo S J, Anderson L C, Green T L, McGarr T, Wells G, Winter S S. Assessing Healthspan and Lifespan Measures in Aging Mice: Optimization of Testing Protocols, Replicability, and Rater Reliability. Current protocols in mouse biology. 2018; 8(2):e45. doi: 10.1002/cpmo.45. PubMed PMID: 29924918.
117. Pathak G, Ibrahim B A, McCarthy S A, Baker K, Kelly M P. Amphetamine sensitization in mice is sufficient to produce both manic- and depressive-related behaviors as well as changes in the functional connectivity of corticolimbic structures. Neuropharmacology. 2015; 95:434-47. doi: 10.1016/j.neuropharm.2015.04.026. PubMed PMID: 25959066.
118. Frankland P W, Bontempi B. The organization of recent and remote memories. Nature reviews Neuroscience. 2005; 6(2):119-30. doi: 10.1038/nm607. PubMed PMID: 15685217.
119. Kitamura T, Ogawa S K, Roy D S, Okuyama T, Morrissey M D, Smith L M, Redondo R L, Tonegawa S. Engrams and circuits crucial for systems consolidation of a memory. Science. 2017; 356(6333):73-8. doi: 10.1126/science.aam6808. PubMed PMID: 28386011; PubMed Central PMCID: PMC5493329.
120. Johnson J D, McGhee A K. Electrophysiological evidence for strategically orienting retrieval toward the specific age of a memory. Brain and cognition. 2015; 100:41-8. doi: 10.1016/j.bandc.2015.09.007. PubMed PMID: 26453976.
121. Kelly M P, Cheung Y F, Favilla C. Siegel S J, Kanes S J, Houslay M D, Abel T, Kelly M P, Cheung Y F, Favilla C, Siegel S J, Kanes S J, Houslay M D, Abel T. Constitutive activation of the G-protein subunit Galphas within forebrain neurons causes PKA-dependent alterations in fear conditioning and cortical Arc mRNA expression. Learning & Memory. 2008; 15(2):75-83.
122. Kelly M P, Isiegas C, Cheung Y F, Tokarczyk J, Yang X, Esposito M F, Rapoport D A, Fabian S A, Siegel S J, Wand G. Houslay M D, Kanes S J, Abel T, Abel T. Constitutive activation of Galphas within forebrain neurons causes deficits in sensorimotor gating because of PKA-dependent decreases in cAMP. Neuropsychopharmacology. 2007; 32(3):577-88.
123. Ceyhan 0, Birsoy K, Hoffman C S. Identification of biologically active PDE11-selective inhibitors using a yeast-based high-throughput screen. Chemistry & biology. 2012; 19(1):155-63. doi: 10.1016/j.chembiol.2011.12.010. PubMed PMID: 22284362.
124. Douglas L A, Varlinskaya E I, Spear L P. Rewarding properties of social interactions in adolescent and adult male and female rats: impact of social versus isolate housing of subjects and partners. Developmental psychobiology. 2004; 45(3):153-62. doi: 10.1002/dev.20025. PubMed PMID: 15505797.
125. U.S. Pat. No. 9,173,884.
126. Fawcett, L.; Baxendale, R.; Stacey, P.; McGrouther, C.; Harrow, I.; Soderling, S.; Hetman, J.; Beavo, J. A.; Phillips, S. C. Molecular cloning and characterization of a distinct human phosphodiesterase gene family: PDE11A. Proc. Natl. Acad. Sci. USA 2000, 97, 3702-3707.
127. Yuasa, K.; Ohgaru, T.; Asahina, M.; Omori, K. Identification of rat cyclic nucleotide phosphodiesterase 11A (PDE11A): comparison of rat and human PDE11A splicing variants. Eur. J. Biochem., 2001, 268, 4440-4448.
128. Yuasa, K.; Kanoh, Y.; Okumura, K.; Omori, K., Genomic organization of the human phosphodiesterase PDE11A gene. Evolutionary relatedness with other PDEs containing GAF domains. Eur. J. Biochem., 2001. 268, 168-78.
129. Hegde, S.; Capell, W. R.; Ibrahim, B. A.; Klett, J.; Patel, N. S.; Sougiannis, A. T.; Kelly, M. P. Phosphodiesterase 11A (PDE11A), Enriched in Ventral Hippocampus Neurons, is Required for Consolidation of Social but not Nonsocial Memories in Mice. Neuropsychopharmacology, 2016, 41, 2920-2931.
130. Kelly, M. P.; Logue, S. F.; Brennan, J. E.; Brandon, N. J. Phosphodiesterase 1 A in brain is enriched in ventral hippocampus and deletion causes psychiatric disease-related phenotypes. Proc. Natl. Acad. Sci. USA, 2010, 107, 8457-8462.
131. Pilarzyk, K.; Porcher, L.; Capell, W. R.; Burbano, S. D.; Davis, J.; Fisher, J. L.; Gomy, N.; Petrolle, S.; Kelly, M. P. Conserved age-related increases in hippocampal PDE11A4 cause unexpected proteinopathies and cognitive decline of social associative memories. Aging Cell, 2022, 21, e13687.
132. Kelly, M. P. Does phosphodiesterase 11A (PDE11A) hold promise as a future therapeutic target?Curr. Pharm. Design, 2015, 21, 389-416.
133. Kelly, M. P.; Adamowicz, W.; Bove, S.; hartman, A. J.; Abigail, M.; Pathak, G.; Reinhart, V.; Romegialli, A.; Kleiman, R. Select 3′,5′-cyclic nucleotide phosphodiesterases exhibit altered expression in the aged rodent brain. Cell Signal, 2014, 26, 383-397.
134. Jager, R.; Russwurm, C.; Schwede, F.; Geniesser, H. G.; Kiesling, D.; Russwurm, M. Activation of PDE10 and PDE11 phosphodiesterases. J. Biol. Chem., 2012. 287, 1210-1219.
135. Kelly, M. P., A Role for Phosphodiesterase 11A (PDE11A) in the Formation of Social Memories and the Stabilization of Mood. Adv. Neurobiol., 2017, 17, 201-230.
136. Rotella, D. P. Phosphodiesterases. Comprehensive Medicinal Chemistry, second edition; Taylor, J. B., Triggle, D. J. eds, Elsevier: Oxford U K, 2007, pp 919-957.
137. Bonkale, W. L.; Cowburn, R. F.; Ohm, T. G.; Bogdanovic, N.; Fastbom, J., A quantitative autoradiographic study of [3H]cAMP binding to cytosolic and particulate protein kinase A in postmortem brain staged for Alzheimer's disease neurofibrillary changes and amyloid deposits, Brain Res., 1999, 818, 383-396.
138. Titus, D. J.; Furones, C.; Kang, Y.; Atkins, C. M., Age-dependent alterations in cAMP signaling contribute to synaptic plasticity deficits following traumatic brain injury. Neuroscience, 2013, 231, 182-94.
139. Pilarzyk, K.; Klett, J.; Pena, E. A.; Porcher, L.; Smith, A. J.; Kelly, M. P., Loss of Function of Phosphodiesterase 11A4 Shows that Recent and Remote Long-Term Memories Can Be Uncoupled, Curr. Biol., 2019, 29, 2307-2321.
140. Baillie, G. S.; Tejeda, G. S.; Kelly, M. P., Therapeutic targeting of 3′,5′-cyclic nucleotide phosphodiesterases: inhibition and beyond, Nature Reviews Drug Discovery, 2019, 18, 770-796.
141. Esin, R. G.; Safina, D. R.; Khakimova, A. R.; Esin, O R., Neuroinflammation and Neuropathology, Neurosci. Behav. Physiol. 2022, 52, 196-201.
142. Jankowska, A.; Pawlowski, M.; Chlon-Rzepa, G., Diabetic theory in anti-Alzheimer's drug research and development. Part 2: Therapeutic potential of cAMP-specific phosphodiesterase inhibitors, Curr. Med. Chem. 2021, 28, 3535-3553.
143. Francis, S. H, Phosphodiesterase 11 (PDE11): Is it a player in human testicular function?, Int. J. Impotence Res. 2005, 17, 467-468.
144. Weeks, J. L.; Corbin, J. D.; Francis, S. H., Interactions between cyclic nucleotide phosphodiesterase 11 catalytic site and substrates or tadalafil and role of a critical Gln-869 hydrogen bond, J. Pharmacol. Exp. Ther. 2009, 331, 133141.
145. Wang, Y.; Bear, T.; Bullock, W.; Chen, L.; Guemon, L.; Gunn, D.; Hunyadi, L.;
Kramss, R.; Li, T.; Liang, S., Selective PDE11A inhibitors as glucose-dependent insulin secretagogues: synthesis and SAR of 1,8-naphthyridinones, Abstracts of papers, 232nd ACS National Meeting, Sep. 10-14, 2006, MEDI 434.
146. MacKenzie, S. J.; Mitchell, L.; Kewney, J, Pyrazolopyrimidines as phosphodiesterase inhibitors for use in therapy of schizophrenia, WO2008056176, 2008.
147. Ceyhan 0.; Birsoy K.; Hoffman C. S. (2012). Identification of biologically active PDE11-selective inhibitors using a yeast-based high throughput screen, Chemistry & Biology, 2012, 19, 155-163.
148. Hoffman, C. S.; Winston, F., Isolation and characterization of mutants constitutive for expression of the fbp1 gene of Schizosaccharomyces pombe, Genetics, 1990, 124, 807-816.
149. Gutz, H.; Heslot, H.; Leupold, U.; Loprieno, N., Schizosaccharomyces pombe, in Handbook of Genetics, R. C. King, Editor. 1974, Plenum Press, New York, 395-446.
150. Volochnyuk, D. M.; Ryabukhin, S. V.; Plaskon, A. S.; Dmytriv, Y. V.; Grygorenko, 0.0; Mykailiuk, P. K.; Krotko, D. G.; Pushechnikov, A.; Tolmachev, A. A, Approach to the library of fused pyridine-4-carboxylic acids by Combes-type reaction of acyl pyruvates and electron-rich amino heterocycles, J. Combinatorial Chem. 2010, 12, 510-517.
151. Akkari, R.; Alvey, L. J.; Bock, X. M.; Brown, B. S.; Claes, P. I. R.; Cowart, M. D.; DeLemos, E.; Desroy, N.; Duthion, B.; Gfesser, G. A.; Gosmini, R. L. M.; Housseman, C. G.; Jansen, K. K.; Ji, J.; Kym, P. S.; LeFrancois, J.-M.; Mammoliti, 0.; Menet, C. J. M.; Meyaro, N. M.; Newsome, G. J.; Palisse, A. M.; Patel, S. V.; Pizzonero, M. R.; Shrestha, A.; Swift, E. C.; van der Plas, S. E.; Wang, X.; DeBlieck, A, N-sulfonylated pyrazolo[3,4-B]pyridin-6-carboxamide and methods of use, WO2017060874, 2017.
152. Oniciu, D. C.; Dasseuz, J. L. H.; Barbaras, R.; Kochubey, V.; Kovalsky, D.; Rodin, O. G.; Geoffroy, O., Compounds, compositions and methods useful for cholesterol modification, WO2012054535, 2012.
153. Menniti, F. S.; Chappie, T. A; Schmidt, C. J. PDE10A inhibitors-clinical failure or a window into antipsychotic drug action. Front. Neurosci. 2021, 14, 600178.
154. Weeks, J. L.; Blount, M. A.; Beasley, A.; Zoraghi, R.; Thomas, M. K.; Sekhar, K. R.; Corbin, J. D.; Francis, S. H. Radiolabeled ligand binding to the catalytic or allosteric sites of PDE5 and PDE11. Meth. Mol. Biol. 2005, 307, 239-262.
155. Pilarzyk, K.; Porcher, L.; Capell, W. R.; Burbano, S. D.; Davis, J.; Fisher, J. L.; Gomy, N.; Petrolle, S.; Kelly, M. P. Conserved age-related increases in hippocampal PDE11A4 cause unexpected pro-teinopathies and cognitive decline of social associative memories. Aging Cell 2022, 21, e13687.
156. Smith, A. J.; Farmer, R.; Pilarzyk, K.; Porcher, L.; Kelly, M. P. A genetic basis for friendship?Ho-mophily for membrane-associated PDE11A-cAMP-CREB signaling in CA1 of hippocampus dictates mutual social preference in male and female mice. Mol. Psychiatry 2021, 26, 7107-7117.

1. A compound of formula (A), or a pharmaceutically acceptable salt, solvate, hydrate, cocrystal, or prodrug thereof: 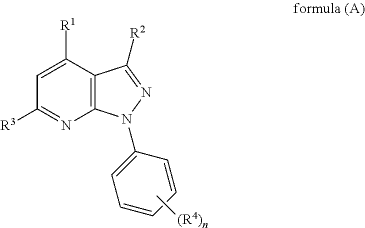
wherein in formula (A):
R¹ is selected from 5-membered heterocycle, 5-membered heteroaryl, 6-membered heterocycle, and 6-membered heteroaryl; or R¹ 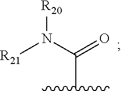
R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
R₂₀ is selected from H, optionally substituted alkyl, and optionally substituted haloalkyl;
R₂₁ is selected from optionally substituted alkyl and optionally substituted haloalkyl;
or R₂₀ and R₂₁ are joined to form a 4-7 membered ring with the nitrogen to which they are each bound;
n is an integer selected from 0-5; and
t is 1 or 2.

2. The compound of claim 1, wherein the compound of formula (A) is a compound of formula (I), or a pharmaceutically acceptable salt, solvate, hydrate, cocrystal, or prodrug thereof: 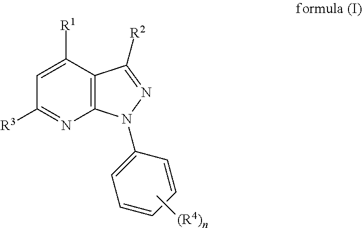
wherein in formula (I):
R¹ and R³ are independently at each occurrence a 5-membered heterocycle, 5-membered heteroaryl, a 6-membered heterocycle, or 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

3. The compound of claim 1 or 2, wherein R¹ is a 5-membered heteroaryl ring, optionally selected from thiazole, oxazole, imidazole, thiadiazole, oxadiazole, and triazole.

4. The compound of any one of claims 1 to 3, or pharmaceutically acceptable salt, solvate, hydrate, cocrystal, or prodrug thereof, wherein the compound is a compound of formula (II): 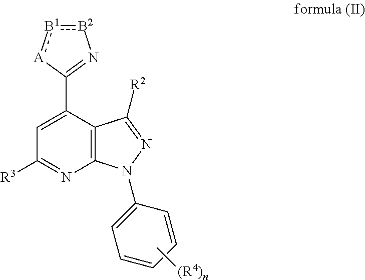
wherein in formula (II):
A is S, NR⁵, N, or O, optionally A is S or 0;
B¹ and B² are independently at each occurrence selected from N, O, or CRS, wherein B¹ is not O when either A or B² is O; and
each R⁵ is independently at each occurrence selected from hydrogen, C₁-C₆ alkyl (e.g., C₁-C₃ alkyl), and C₁-C₃ haloalkyl (e.g., CH₂F, CHF₂, CF₃);
R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
n is an integer selected from 0-5; and
t is 1 or 2.

5. The compound of claim 4, wherein R¹ is selected from 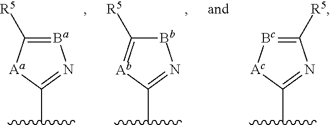
wherein:
A^{a} is O or S, and B^{a} is N or CH;
A^{b} is N, and Bb is O or S;
A^{c} is O or S, and BC is N; and
R⁵ is selected from hydrogen, C₁-C₃ alkyl (e.g., methyl, ethyl, propyl, isopropyl), and C₁-C₃ haloalkyl (e.g., CH₂F, CHF₂, or CF₃).

6. The compound of claim 4 or 5, wherein R¹ is selected from 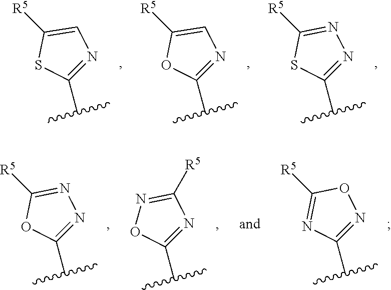
and R⁵ is selected from H, methyl, ethyl, propyl, isopropyl, CH₂F, and CHF₂.

7. The compound of any one of claims 4-6, wherein R¹ is selected from

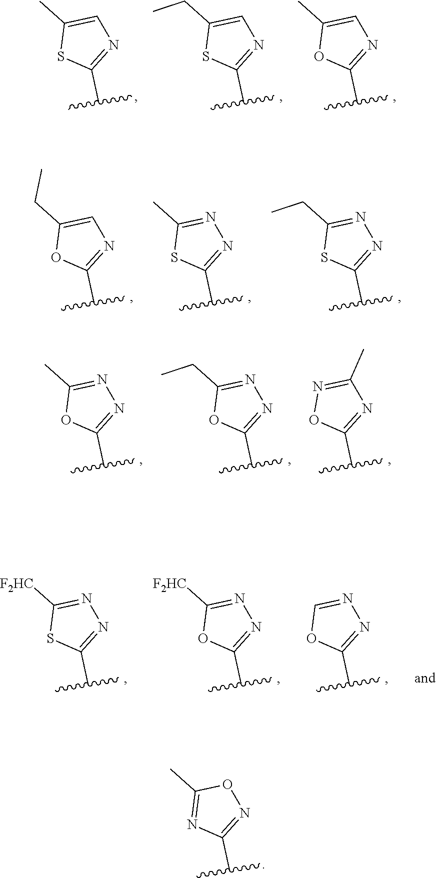

8. The compound of any one of claims 1-7, wherein R₂ is selected from hydrogen, C₃-C₆ cycloalkyl, C₁-C₆ alkyl (e.g. C₁-C₄ straight chain alkyl, C₃-C₆ branched alkyl), —CF₃, —CH₂F, —CHF₂, and —C(R⁶R⁷)ₚX;
wherein, p is an integer from 1-3;
R⁶ and R⁷ are each independently selected from hydrogen, C₁-C₃ alkyl, and C₃-C₆ cycloalkyl;
X is selected from —OH, —CN, —OR^{a}, and NR⁹R¹⁰;
R⁸ is selected from C₁-C₃ alkyl (e.g., C₁-C₃ straight or branched alkyl), and C₃-C₆ cycloalkyl; and
R⁹ and R¹⁰ are each independently selected from hydrogen, C₁-C₃ alkyl, and C₃-C₆ cycloalkyl.

9. The compound of claim 8, wherein R₂ is selected from hydrogen, C₁-C₄ alkyl (e.g., methyl, ethyl, propyl, isopropyl, butyl, tertbutyl), —CF₃, —CH₂F, and —CHF₂.

10. The compound of claim 8 or 9, wherein R₂ is selected from H, —CH₃, —CF₃, and —CHF₂.

11. The compound of any one of claims 1-10, wherein is R³ is a 5-membered heteroaryl ring, optionally selected from pyrrole, thiophene, furan, pyrazole, thiazole, isothiazole, oxazole, isoxazole, and imidazole.

12. The compound of claim 11, wherein R³ is selected from 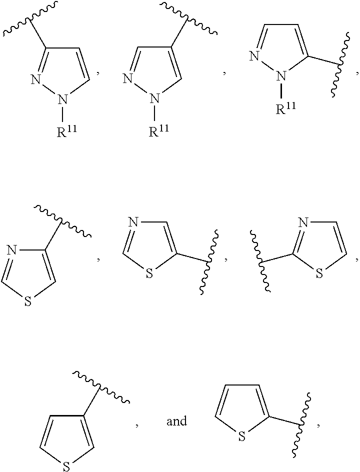
wherein R¹¹ is selected from hydrogen, C₁-C₃ alkyl (e.g., methyl, ethyl, propyl, isopropyl), C₃-C₆ cycloalkyl, CF₃, CH₂CF₃, CH₂CHF₂, and CH₂CH₂F.

13. The compound of claim 12, wherein R³ is selected from

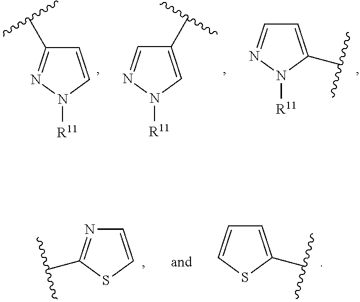

14. The compound of claim 12 or 13, wherein R¹¹ is selected from hydrogen, methyl, ethyl, propyl, isopropyl, —CF₃, and —CH₂CF₃.

15. The compound of any one of claims 11-14, wherein R³ is selected from

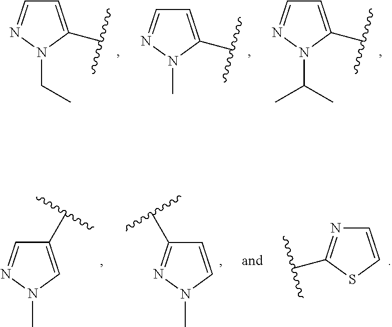

16. The compound of any one of claims 11-15, wherein R³ is selected from

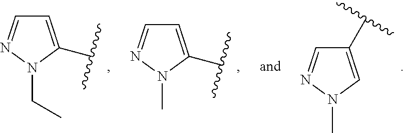

17. The compound of any one of claims 1-16, wherein R⁴ is selected from hydrogen, C₁-C₆ alkyl, and halo (e.g., F or Cl).

18. The compound of any one of claims 1-17, wherein n is 0, 1, or 2.

19. The compound of claim 18, wherein n is 0 or 1.

20. The compound of any one of claims 1-19, wherein R⁴ is selected from hydrogen, 4-fluoro, 3-fluoro, 2-fluoro, 2-chloro, and 4-chloro.

21. The compound of any one of claims 1-20, wherein R₄ is selected from hydrogen, 4-fluoro, 2-fluoro, and 2-chloro.

22. The compound of any one of claims 1-18, wherein n is 2 and each R⁴ is 2-fluoro and 4-fluoro.

23. The compound of any one of claims 1-4, wherein the compound of formula (A), formula (I), or formula (II) is a compound having any one of formula 1001-1080, 1097-1176, 1193-1272, 1289-1368, 1385-1729, 4001-4080, 4097-4176, 4193-4272, 4289-4368, 4385-4729, or a pharmaceutically acceptable salt, solvate, hydrate, cocrystal, or prodrug thereof:
Formula (Ia) 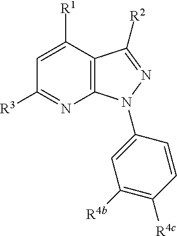
formula (Ib)

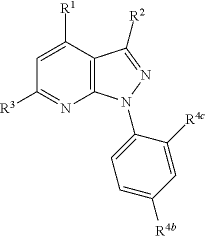

| Cpd. # | Cpd. # | | | | | |
|---|---|---|---|---|---|---|
| Formula (Ia) | Formula (Ib) | R¹ | R² | R³ | R^{4b} | R^{4c} |
| 1001 | 4001 | 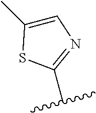 [SMILES: *c1ncc(C)s1] | H | 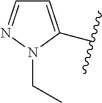 [SMILES: *c1ccnn1CC] | H | H |
| 1002 | 4002 | 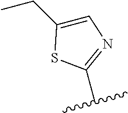 [SMILES: *c1ncc(CC)s1] | H | 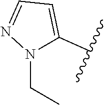 [SMILES: *c1ccnn1CC] | H | H |
| 1003 | 4003 | 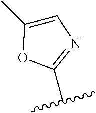 [SMILES: *c1ncc(C)o1] | H | 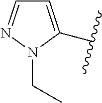 [SMILES: *c1ccnn1CC] | H | H |
| 1004 | 4004 | 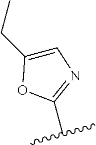 [SMILES: *c1ncc(CC)o1] | H | 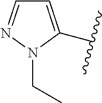 [SMILES: *c1ccnn1CC] | H | H |
| 1005 | 4005 | 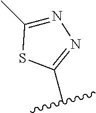 [SMILES: *c1nnc(C)s1] | H | 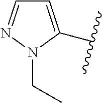 [SMILES: *c1ccnn1CC] | H | H |
| 1006 | 4006 | 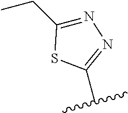 [SMILES: *c1nnc(CC)s1] | H | 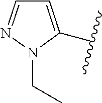 [SMILES: *c1ccnn1CC] | H | H |
| 1007 | 4007 | 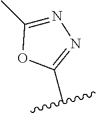 [SMILES: *c1nnc(C)o1] | H | 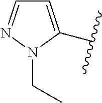 [SMILES: *c1ccnn1CC] | H | H |
| 1008 | 4008 | 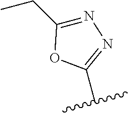 [SMILES: *c1nnc(CC)o1] | H | 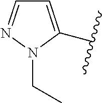 [SMILES: *c1ccnn1CC] | H | H |
| 1009 | 4009 | 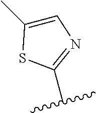 [SMILES: *c1ncc(C)s1] | CH₃ | 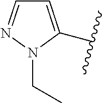 [SMILES: *c1ccnn1CC] | H | H |
| 1010 | 4010 | 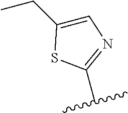 [SMILES: *c1ncc(CC)s1] | CH₃ | 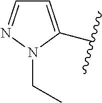 [SMILES: *c1ccnn1CC] | H | H |
| 1011 | 4011 | 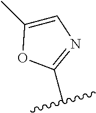 [SMILES: *c1ncc(C)o1] | CH₃ | 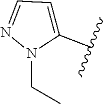 [SMILES: *c1ccnn1CC] | H | H |
| 1012 | 4012 | 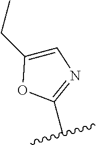 [SMILES: *c1ncc(CC)o1] | CH₃ | 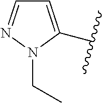 [SMILES: *c1ccnn1CC] | H | H |
| 1013 | 4013 | 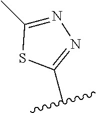 [SMILES: *c1nnc(C)s1] | CH₃ | 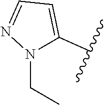 [SMILES: *c1ccnn1CC] | H | H |
| 1014 | 4014 | 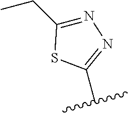 [SMILES: *c1nnc(CC)s1] | CH₃ | 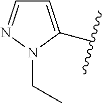 [SMILES: *c1ccnn1CC] | H | H |
| 1015 | 4015 | 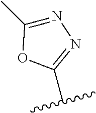 [SMILES: *c1nnc(C)o1] | CH₃ | 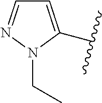 [SMILES: *c1ccnn1CC] | H | H |
| 1016 | 4016 | 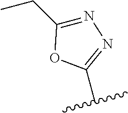 [SMILES: *c1nnc(CC)o1] | CH | 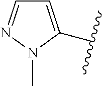 [SMILES: *c1ccnn1C] | H | H |
| 1017 | 4017 | 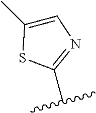 [SMILES: *c1ncc(C)s1] | H | 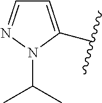 [SMILES: *c1ccnn1C(C)C] | H | H |
| 1018 | 4018 | 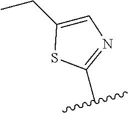 [SMILES: *c1ncc(CC)s1] | H | 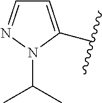 [SMILES: *c1ccnn1C(C)C] | H | H |
| 1019 | 4019 | 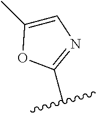 [SMILES: *c1ncc(C)o1] | H | 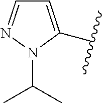 [SMILES: *c1ccnn1C(C)C] | H | H |
| 1020 | 4020 | 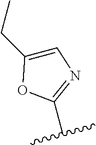 [SMILES: *c1ncc(CC)o1] | H | 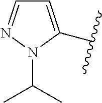 [SMILES: *c1ccnn1C(C)C] | H | H |
| 1021 | 4021 | 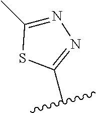 [SMILES: *c1nnc(C)s1] | H | 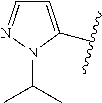 [SMILES: *c1ccnn1C(C)C] | H | H |
| 1022 | 4022 | 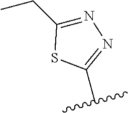 [SMILES: *c1nnc(CC)s1] | H | 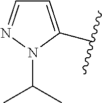 [SMILES: *c1ccnn1C(C)C] | H | H |
| 1023 | 4023 | 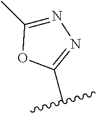 [SMILES: *c1nnc(C)o1] | H | 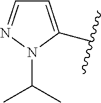 [SMILES: *c1ccnn1C(C)C] | H | H |
| 1024 | 4024 | 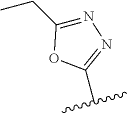 [SMILES: *c1nnc(CC)o1] | H | 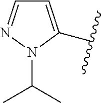 [SMILES: *c1ccnn1C(C)C] | H | H |
| 1025 | 4025 | 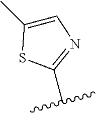 [SMILES: *c1ncc(C)s1] | CH₃ | 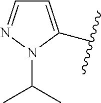 [SMILES: *c1ccnn1C(C)C] | H | H |
| 1026 | 4026 | 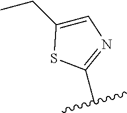 [SMILES: *c1ncc(CC)s1] | CH₃ | 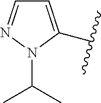 [SMILES: *c1ccnn1C(C)C] | H | H |
| 1027 | 4027 | 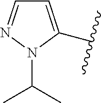 [SMILES: *c1ccnn1C(C)C] | CH₃ | 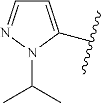 [SMILES: *c1ccnn1C(C)C] | H | H |
| 1028 | 4028 | 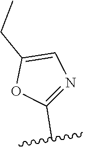 [SMILES: *c1ncc(CC)o1] | CH₃ | 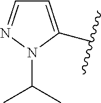 [SMILES: *c1ccnn1C(C)C] | H | H |
| 1029 | 4029 | 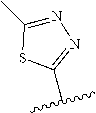 [SMILES: *c1nnc(C)s1] | CH₃ | 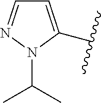 [SMILES: *c1ccnn1C(C)C] | H | H |
| 1030 | 4030 | 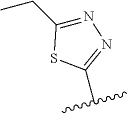 [SMILES: *c1nnc(CC)s1] | CH₃ | 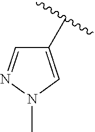 [SMILES: *c1cnn(C)c1] | H | H |
| 1031 | 4031 | 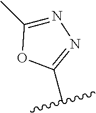 [SMILES: *c1nnc(C)o1] | CH₃ | 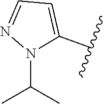 [SMILES: *c1ccnn1C(C)C] | H | H |
| 1032 | 4032 | 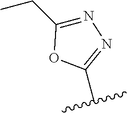 [SMILES: *c1nnc(CC)o1] | CH₃ | 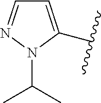 [SMILES: *c1ccnn1C(C)C] | H | H |
| 1033 | 4033 | 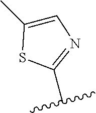 [SMILES: *c1ncc(C)s1] | H | 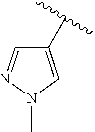 [SMILES: *c1cnn(C)c1] | H | H |
| 1034 | 4034 | 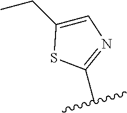 [SMILES: *c1ncc(CC)s1] | H | 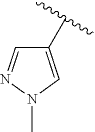 [SMILES: *c1cnn(C)c1] | H | H |
| 1035 | 4035 | 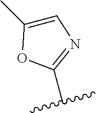 [SMILES: *c1ncc(C)o1] | H | 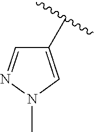 [SMILES: *c1cnn(C)c1] | H | H |
| 1036 | 4036 | 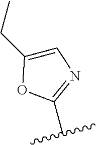 [SMILES: *c1ncc(CC)o1] | H | 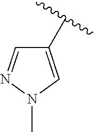 [SMILES: *c1cnn(C)c1] | H | H |
| 1037 | 4037 | 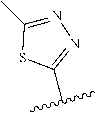 [SMILES: *c1nnc(C)s1] | H | 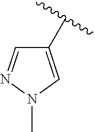 [SMILES: *c1cnn(C)c1] | H | H |
| 1038 | 4038 | 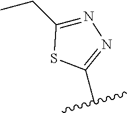 [SMILES: *c1nnc(CC)s1] | H | 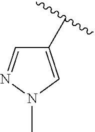 [SMILES: *c1cnn(C)c1] | H | H |
| 1039 | 4039 | 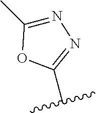 [SMILES: *c1nnc(C)o1] | H | 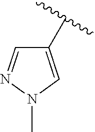 [SMILES: *c1cnn(C)c1] | H | H |
| 1040 | 4040 | 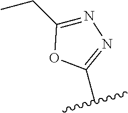 [SMILES: *c1nnc(CC)o1] | H | 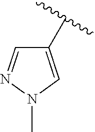 [SMILES: *c1cnn(C)c1] | H | H |
| 1041 | 4041 | 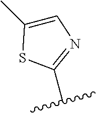 [SMILES: *c1ncc(C)s1] | CH₃ | 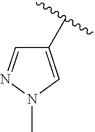 [SMILES: *c1cnn(C)c1] | H | H |
| 1042 | 4042 | 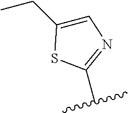 [SMILES: *c1ncc(CC)s1] | CH₃ | 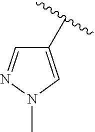 [SMILES: *c1cnn(C)c1] | H | H |
| 1043 | 4043 | 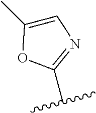 [SMILES: *c1ncc(C)o1] | CH₃ | 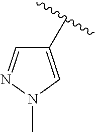 [SMILES: *c1cnn(C)c1] | H | H |
| 1044 | 4044 | 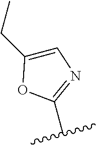 [SMILES: *c1ncc(CC)o1] | CH₃ | 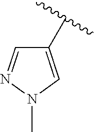 [SMILES: *c1cnn(C)c1] | H | H |
| 1045 | 4045 | 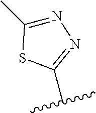 [SMILES: *c1nnc(C)s1] | CH₃ | 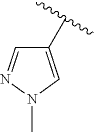 [SMILES: *c1cnn(C)c1] | H | H |
| 1046 | 4046 | 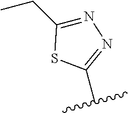 [SMILES: *c1nnc(CC)s1] | CH₃ | 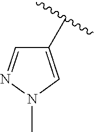 [SMILES: *c1cnn(C)c1] | H | H |
| 1047 | 4047 | 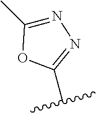 [SMILES: *c1nnc(C)o1] | CH₃ | 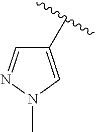 [SMILES: *c1cnn(C)c1] | H | H |
| 1048 | 4048 | 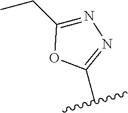 [SMILES: *c1nnc(CC)o1] | CH₃ | 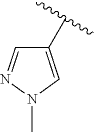 [SMILES: *c1cnn(C)c1] | H | H |
| 1049 | 4049 | 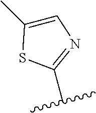 [SMILES: *c1ncc(C)s1] | H | 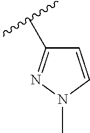 [SMILES: *c1ccn(C)n1] | H | H |
| 1050 | 4050 | 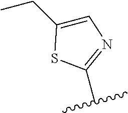 [SMILES: *c1ncc(CC)s1] | H | 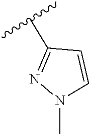 [SMILES: *c1ccn(C)n1] | H | H |
| 1051 | 4051 | 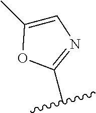 [SMILES: *c1ncc(C)o1] | H | 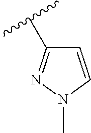 [SMILES: *c1ccn(C)n1] | H | H |
| 1052 | 4052 | 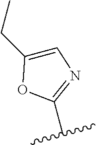 [SMILES: *c1ncc(CC)o1] | H | 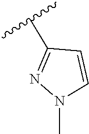 [SMILES: *c1ccn(C)n1] | H | H |
| 1053 | 4053 | 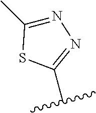 [SMILES: *c1nnc(C)s1] | H | 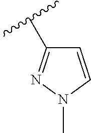 [SMILES: *c1ccn(C)n1] | H | H |
| 1054 | 4054 | 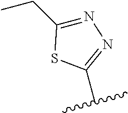 [SMILES: *c1nnc(CC)s1] | H | 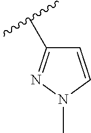 [SMILES: *c1ccn(C)n1] | H | H |
| 1055 | 4055 | 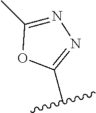 [SMILES: *c1nnc(C)o1] | H | 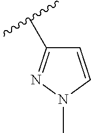 [SMILES: *c1ccn(C)n1] | H | H |
| 1056 | 4056 | 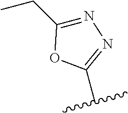 [SMILES: *c1nnc(CC)o1] | H | 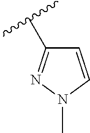 [SMILES: *c1ccn(C)n1] | H | H |
| 1057 | 4057 | 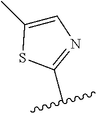 [SMILES: *c1ncc(C)s1] | CH₃ | 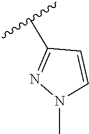 [SMILES: *c1ccn(C)n1] | H | H |
| 1058 | 4058 | 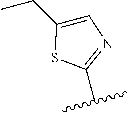 [SMILES: *c1ncc(CC)s1] | CH₃ | 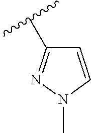 [SMILES: *c1ccn(C)n1] | H | H |
| 1059 | 4059 | 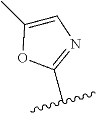 [SMILES: *c1ncc(C)o1] | CH₃ | 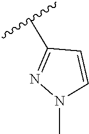 [SMILES: *c1ccn(C)n1] | H | H |
| 1060 | 4060 | 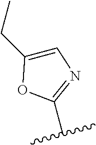 [SMILES: *c1ncc(CC)o1] | CH₃ | 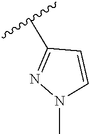 [SMILES: *c1ccn(C)n1] | H | H |
| 1061 | 4061 | 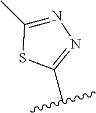 [SMILES: *c1nnc(C)s1] | CH₃ | 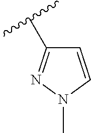 [SMILES: *c1ccn(C)n1] | H | H |
| 1062 | 4062 | 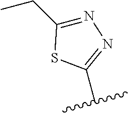 [SMILES: *c1nnc(CC)s1] | CH₃ | 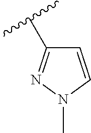 [SMILES: *c1ccn(C)n1] | H | H |
| 1063 | 4063 | 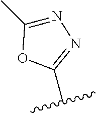 [SMILES: *c1nnc(C)o1] | CH₃ | 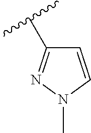 [SMILES: *c1ccn(C)n1] | H | H |
| 1064 | 4064 | 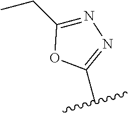 [SMILES: *c1nnc(CC)o1] | CH₃ | 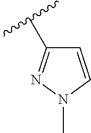 [SMILES: *c1ccn(C)n1] | H | H |
| 1065 | 4065 | 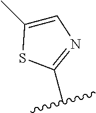 [SMILES: *c1ncc(C)s1] | H | 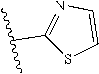 [SMILES: *c1nccs1] | H | H |
| 1066 | 4066 | 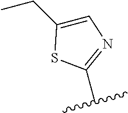 [SMILES: *c1ncc(CC)s1] | H | 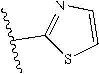 [SMILES: *c1nccs1] | H | H |
| 1067 | 4067 | 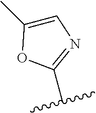 [SMILES: *c1ncc(C)o1] | H | 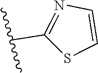 [SMILES: *c1nccs1] | H | H |
| 1068 | 4068 | 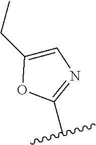 [SMILES: *c1ncc(CC)o1] | H | 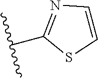 [SMILES: *c1nccs1] | H | H |
| 1069 | 4069 | 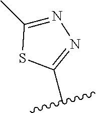 [SMILES: *c1nnc(C)s1] | H | 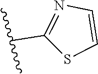 [SMILES: *c1nccs1] | H | H |
| 1070 | 4070 | 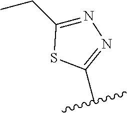 [SMILES: *c1nnc(CC)s1] | H | 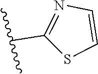 [SMILES: *c1nccs1] | H | H |
| 1071 | 4071 | 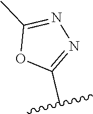 [SMILES: *c1nnc(C)o1] | H | 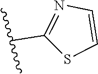 [SMILES: *c1nccs1] | H | H |
| 1072 | 4072 | 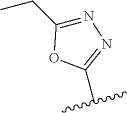 [SMILES: *c1nnc(CC)o1] | H | 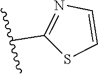 [SMILES: *c1nccs1] | H | H |
| 1073 | 4073 | 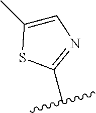 [SMILES: *c1ncc(C)s1] | CH₃ | 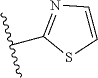 [SMILES: *c1nccs1] | H | H |
| 1074 | 4074 | 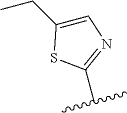 [SMILES: *c1ncc(CC)s1] | CH₃ | 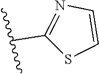 [SMILES: *c1nccs1] | H | H |
| 1075 | 4075 | 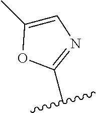 [SMILES: *c1ncc(C)o1] | CH₃ | 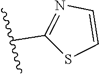 [SMILES: *c1nccs1] | H | H |
| 1076 | 4076 | 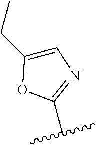 [SMILES: *c1ncc(CC)o1] | CH₃ | 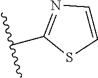 [SMILES: *c1nccs1] | H | H |
| 1077 | 4077 | 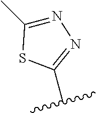 [SMILES: *c1nnc(C)s1] | CH₃ | 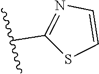 [SMILES: *c1nccs1] | H | H |
| 1078 | 4078 | 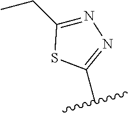 [SMILES: *c1nnc(CC)s1] | CH₃ | 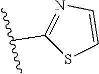 [SMILES: *c1nccs1] | H | H |
| 1079 | 4079 | 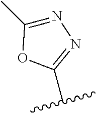 [SMILES: *c1nnc(C)o1] | CH₃ | 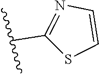 [SMILES: *c1nccs1] | H | H |
| 1080 | 4080 | 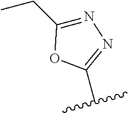 [SMILES: *c1nnc(CC)o1] | CH₃ | 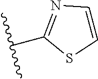 [SMILES: *c1nccs1] | H | H |
| 1097 | 4097 | 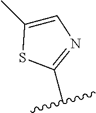 [SMILES: *c1ncc(C)s1] | H | 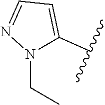 [SMILES: *c1ccnn1CC] | H | F |
| 1098 | 4098 | 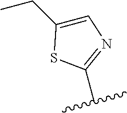 [SMILES: *c1ncc(CC)s1] | H | 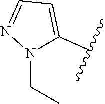 [SMILES: *c1ccnn1CC] | H | F |
| 1099 | 4099 | 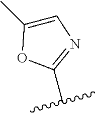 [SMILES: *c1ncc(C)o1] | H | 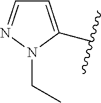 [SMILES: *c1ccnn1CC] | H | F |
| 1100 | 4100 | 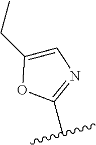 [SMILES: *c1ncc(CC)o1] | H | 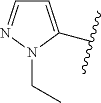 [SMILES: *c1ccnn1CC] | H | F |
| 1101 | 4101 | 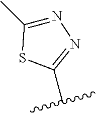 [SMILES: *c1nnc(C)s1] | H | 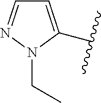 [SMILES: *c1ccnn1CC] | H | F |
| 1102 | 4102 | 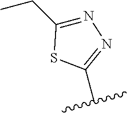 [SMILES: *c1nnc(CC)s1] | H | 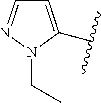 [SMILES: *c1ccnn1CC] | H | F |
| 1103 | 4103 | 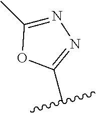 [SMILES: *c1nnc(C)o1] | H | 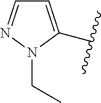 [SMILES: *c1ccnn1CC] | H | F |
| 1104 | 4104 | 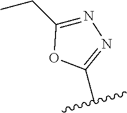 [SMILES: *c1nnc(CC)o1] | H | 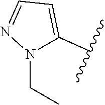 [SMILES: *c1ccnn1CC] | H | F |
| 1105 | 4105 | 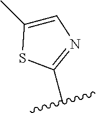 [SMILES: *c1ncc(C)s1] | CH₃ | 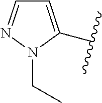 [SMILES: *c1ccnn1CC] | H | F |
| 1106 | 4106 | 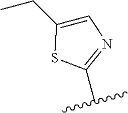 [SMILES: *c1ncc(CC)s1] | CH₃ | 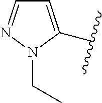 [SMILES: *c1ccnn1CC] | H | F |
| 1107 | 4107 | 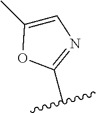 [SMILES: *c1ncc(C)o1] | CH₃ | 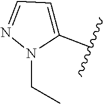 [SMILES: *c1ccnn1CC] | H | F |
| 1108 | 4108 | 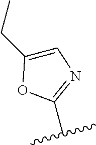 [SMILES: *c1ncc(CC)o1] | CH₃ | 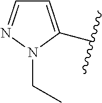 [SMILES: *c1ccnn1CC] | H | F |
| 1109 | 4109 | 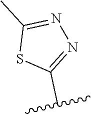 [SMILES: *c1nnc(C)s1] | CH₃ | 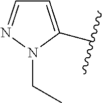 [SMILES: *c1ccnn1CC] | H | F |
| 1110 | 4110 | 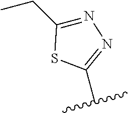 [SMILES: *c1nnc(CC)s1] | CH₃ | 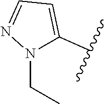 [SMILES: *c1ccnn1CC] | H | F |
| 1111 | 4111 | 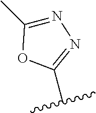 [SMILES: *c1nnc(C)o1] | CH₃ | 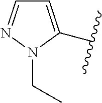 [SMILES: *c1ccnn1CC] | H | F |
| 1112 | 4112 | 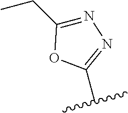 [SMILES: *c1nnc(CC)o1] | CH₃ | 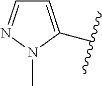 [SMILES: *c1ccnn1C] | H | F |
| 1113 | 4113 | 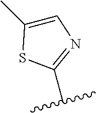 [SMILES: *c1ncc(C)s1] | H | 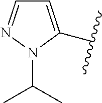 [SMILES: *c1ccnn1C(C)C] | H | F |
| 1114 | 4114 | 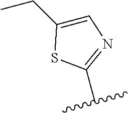 [SMILES: *c1ncc(CC)s1] | H | 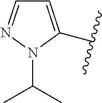 [SMILES: *c1ccnn1C(C)C] | H | F |
| 1115 | 4115 | 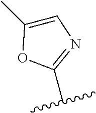 [SMILES: *c1ncc(C)o1] | H | 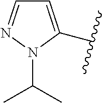 [SMILES: *c1ccnn1C(C)C] | H | F |
| 1116 | 4116 | 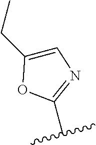 [SMILES: *c1ncc(CC)o1] | H | 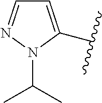 [SMILES: *c1ccnn1C(C)C] | H | F |
| 1117 | 4117 | 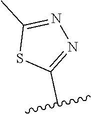 [SMILES: *c1nnc(C)s1] | H | 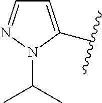 [SMILES: *c1ccnn1C(C)C] | H | F |
| 1118 | 4118 | 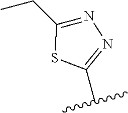 [SMILES: *c1nnc(CC)s1] | H | 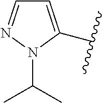 [SMILES: *c1ccnn1C(C)C] | H | F |
| 1119 | 4119 | 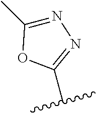 [SMILES: *c1nnc(C)o1] | H | 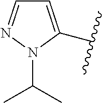 [SMILES: *c1ccnn1C(C)C] | H | F |
| 1120 | 4120 | 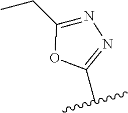 [SMILES: *c1nnc(CC)o1] | H | 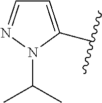 [SMILES: *c1ccnn1C(C)C] | H | F |
| 1121 | 4121 | 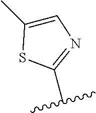 [SMILES: *c1ncc(C)s1] | CH₃ | 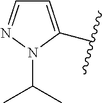 [SMILES: *c1ccnn1C(C)C] | H | F |
| 1122 | 4122 | 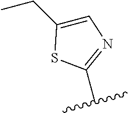 [SMILES: *c1ncc(CC)s1] | CH₃ | 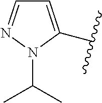 [SMILES: *c1ccnn1C(C)C] | H | F |
| 1123 | 4123 | 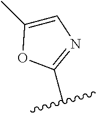 [SMILES: *c1ncc(C)o1] | CH₃ | 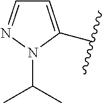 [SMILES: *c1ccnn1C(C)C] | H | F |
| 1124 | 4124 | 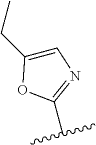 [SMILES: *c1ncc(CC)o1] | CH₃ | 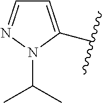 [SMILES: *c1ccnn1C(C)C] | H | F |
| 1125 | 4125 | 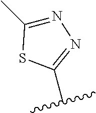 [SMILES: *c1nnc(C)s1] | CH₃ | 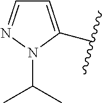 [SMILES: *c1ccnn1C(C)C] | H | F |
| 1126 | 4126 | 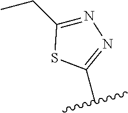 [SMILES: *c1nnc(CC)s1] | CH₃ | 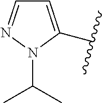 [SMILES: *c1ccnn1C(C)C] | H | F |
| 1127 | 4127 | 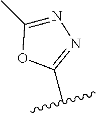 [SMILES: *c1nnc(C)o1] | CH₃ | 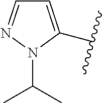 [SMILES: *c1ccnn1C(C)C] | H | F |
| 1128 | 4128 | 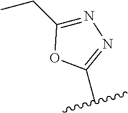 [SMILES: *c1nnc(CC)o1] | CH₃ | 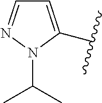 [SMILES: *c1ccnn1C(C)C] | H | F |
| 1129 | 4129 | 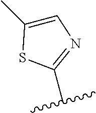 [SMILES: *c1ncc(C)s1] | H | 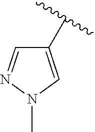 [SMILES: *c1cnn(C)c1] | H | F |
| 1130 | 4130 | 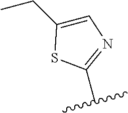 [SMILES: *c1ncc(CC)s1] | H | 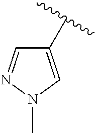 [SMILES: *c1cnn(C)c1] | H | F |
| 1131 | 4131 | 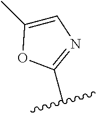 [SMILES: *c1ncc(C)o1] | H | 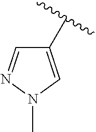 [SMILES: *c1cnn(C)c1] | H | F |
| 1132 | 4132 | 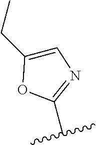 [SMILES: *c1ncc(CC)o1] | H | 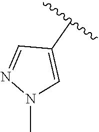 [SMILES: *c1cnn(C)c1] | H | F |
| 1133 | 4133 | 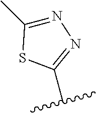 [SMILES: *c1nnc(C)s1] | H | 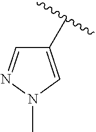 [SMILES: *c1cnn(C)c1] | H | F |
| 1134 | 4134 | 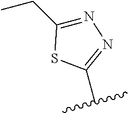 [SMILES: *c1nnc(CC)s1] | H | 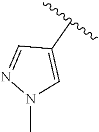 [SMILES: *c1cnn(C)c1] | H | F |
| 1135 | 4135 | 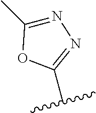 [SMILES: *c1nnc(C)o1] | H | 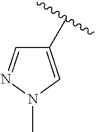 [SMILES: *c1cnn(C)c1] | H | F |
| 1136 | 4136 | 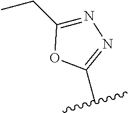 [SMILES: *c1nnc(CC)o1] | H | 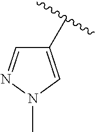 [SMILES: *c1cnn(C)c1] | H | F |
| 1137 | 4137 | 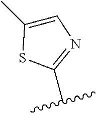 [SMILES: *c1ncc(C)s1] | CH₃ | 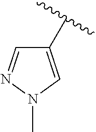 [SMILES: *c1cnn(C)c1] | H | F |
| 1138 | 4138 | 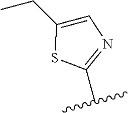 [SMILES: *c1ncc(CC)s1] | CH₃ | 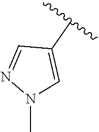 [SMILES: *c1cnn(C)c1] | H | F |
| 1139 | 4139 | 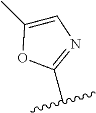 [SMILES: *c1ncc(C)o1] | CH₃ | 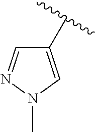 [SMILES: *c1cnn(C)c1] | H | F |
| 1140 | 4140 | 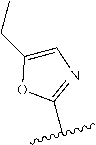 [SMILES: *c1ncc(CC)o1] | CH₃ | 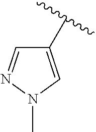 [SMILES: *c1cnn(C)c1] | H | F |
| 1141 | 4141 | 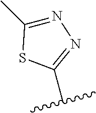 [SMILES: *c1nnc(C)s1] | CH₃ | 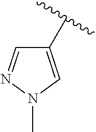 [SMILES: *c1cnn(C)c1] | H | F |
| 1142 | 4142 | 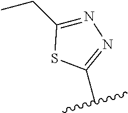 [SMILES: *c1nnc(CC)s1] | CH₃ | 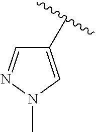 [SMILES: *c1cnn(C)c1] | H | F |
| 1143 | 4143 | 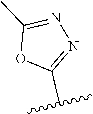 [SMILES: *c1nnc(C)o1] | CH₃ | 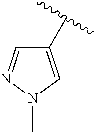 [SMILES: *c1cnn(C)c1] | H | F |
| 1144 | 4144 | 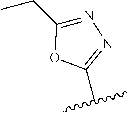 [SMILES: *c1nnc(CC)o1] | CH₃ | 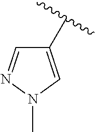 [SMILES: *c1cnn(C)c1] | H | F |
| 1145 | 4145 | 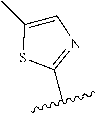 [SMILES: *c1ncc(C)s1] | H | 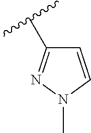 [SMILES: *c1ccn(C)n1] | H | F |
| 1146 | 4146 | 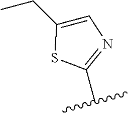 [SMILES: *c1ncc(CC)s1] | H | 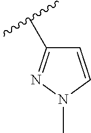 [SMILES: *c1ccn(C)n1] | H | F |
| 1147 | 4147 | 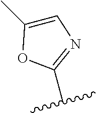 [SMILES: *c1ncc(C)o1] | H | 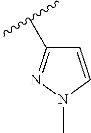 [SMILES: *c1ccn(C)n1] | H | F |
| 1148 | 4148 | 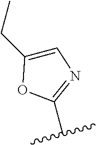 [SMILES: *c1ncc(CC)o1] | H | 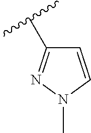 [SMILES: *c1ccn(C)n1] | H | F |
| 1149 | 4149 | 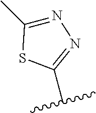 [SMILES: *c1nnc(C)s1] | H | 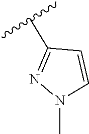 [SMILES: *c1ccn(C)n1] | H | F |
| 1150 | 4150 | 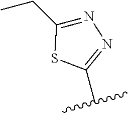 [SMILES: *c1nnc(CC)s1] | H | 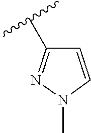 [SMILES: *c1ccn(C)n1] | H | F |
| 1151 | 4151 | 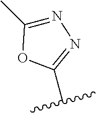 [SMILES: *c1nnc(C)o1] | H | 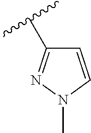 [SMILES: *c1ccn(C)n1] | H | F |
| 1152 | 4152 | 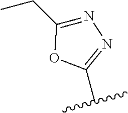 [SMILES: *c1nnc(CC)o1] | H | 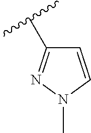 [SMILES: *c1ccn(C)n1] | H | F |
| 1153 | 4153 | 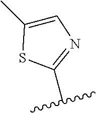 [SMILES: *c1ncc(C)s1] | CH₃ | 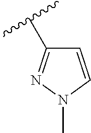 [SMILES: *c1ccn(C)n1] | H | F |
| 1154 | 4154 | 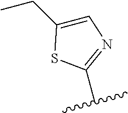 [SMILES: *c1ncc(CC)s1] | CH₃ | 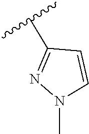 [SMILES: *c1ccn(C)n1] | H | F |
| 1155 | 4155 | 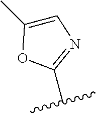 [SMILES: *c1ncc(C)o1] | CH₃ | 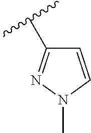 [SMILES: *c1ccn(C)n1] | H | F |
| 1156 | 4156 | 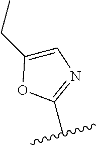 [SMILES: *c1ncc(CC)o1] | CH₃ | 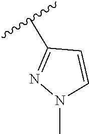 [SMILES: *c1ccn(C)n1] | H | F |
| 1157 | 4157 | 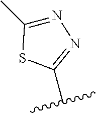 [SMILES: *c1nnc(C)s1] | CH₃ | 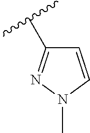 [SMILES: *c1ccn(C)n1] | H | F |
| 1158 | 4158 | 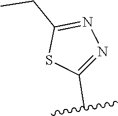 [SMILES: *c1nnc(CC)s1] | CH₃ | 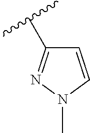 [SMILES: *c1ccn(C)n1] | H | F |
| 1159 | 4159 | 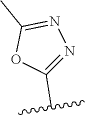 [SMILES: *c1nnc(C)o1] | CH₃ | 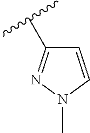 [SMILES: *c1ccn(C)n1] | H | F |
| 1160 | 4160 | 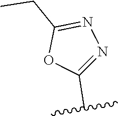 [SMILES: *c1nnc(CC)o1] | CH₃ | 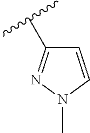 [SMILES: *c1ccn(C)n1] | H | F |
| 1161 | 4161 | 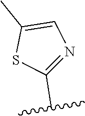 [SMILES: *c1ncc(C)s1] | H | 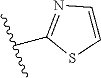 [SMILES: *c1nccs1] | H | F |
| 1162 | 4162 | 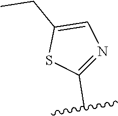 [SMILES: *c1ncc(CC)s1] | H | 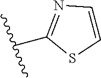 [SMILES: *c1nccs1] | H | F |
| 1163 | 4163 | 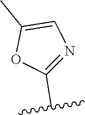 [SMILES: *c1ncc(C)o1] | H | 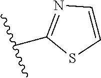 [SMILES: *c1nccs1] | H | F |
| 1164 | 4164 | 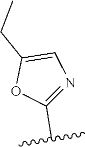 [SMILES: *c1ncc(CC)o1] | H | 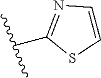 [SMILES: *c1nccs1] | H | F |
| 1165 | 4165 | 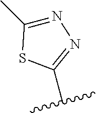 [SMILES: *c1nnc(C)s1] | H | 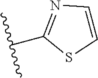 [SMILES: *c1nccs1] | H | F |
| 1166 | 4166 | 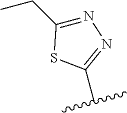 [SMILES: *c1nnc(CC)s1] | H | 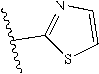 [SMILES: *c1nccs1] | H | F |
| 1167 | 4167 | 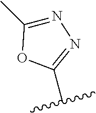 [SMILES: *c1nnc(C)o1] | H | 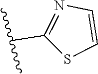 [SMILES: *c1nccs1] | H | F |
| 1168 | 4168 | 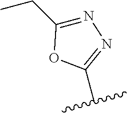 [SMILES: *c1nnc(CC)o1] | H | 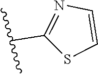 [SMILES: *c1nccs1] | H | F |
| 1169 | 4169 | 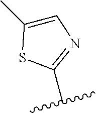 [SMILES: *c1ncc(C)s1] | CH₃ | 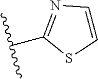 [SMILES: *c1nccs1] | H | F |
| 1170 | 4170 | 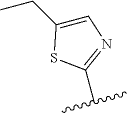 [SMILES: *c1ncc(CC)s1] | CH₃ | 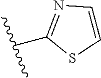 [SMILES: *c1nccs1] | H | F |
| 1171 | 4171 | 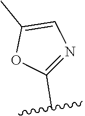 [SMILES: *c1ncc(C)o1] | CH₃ | 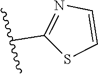 [SMILES: *c1nccs1] | H | F |
| 1172 | 4172 | 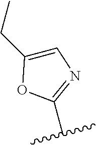 [SMILES: *c1ncc(CC)o1] | CH₃ | 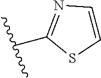 [SMILES: *c1nccs1] | H | F |
| 1173 | 4173 | 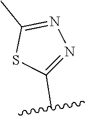 [SMILES: *c1nnc(C)s1] | CH₃ | 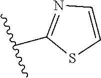 [SMILES: *c1nccs1] | H | F |
| 1174 | 4174 | 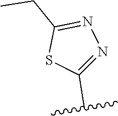 [SMILES: *c1nnc(CC)s1] | CH₃ | 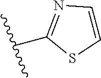 [SMILES: *c1nccs1] | H | F |
| 1175 | 4175 | 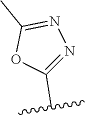 [SMILES: *c1nnc(C)o1] | CH₃ | 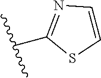 [SMILES: *c1nccs1] | H | F |
| 1176 | 4176 | 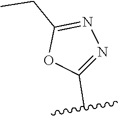 [SMILES: *c1nnc(CC)o1] | CH₃ | 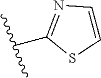 [SMILES: *c1nccs1] | H | F |
| 1193 | 4193 | 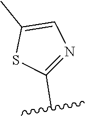 [SMILES: *c1ncc(C)s1] | H | 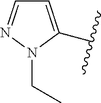 [SMILES: *c1ccnn1CC] | F | H |
| 1194 | 4194 | 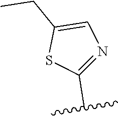 [SMILES: *c1ncc(CC)s1] | H | 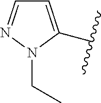 [SMILES: *c1ccnn1CC] | F | H |
| 1195 | 4195 | 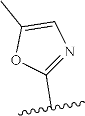 [SMILES: *c1ncc(C)o1] | H | 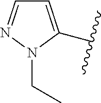 [SMILES: *c1ccnn1CC] | F | H |
| 1196 | 4196 | 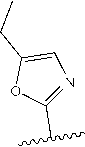 [SMILES: *c1ncc(CC)o1] | H | 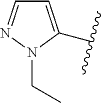 [SMILES: *c1ccnn1CC] | F | H |
| 1197 | 4197 | 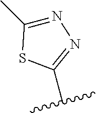 [SMILES: *c1nnc(C)s1] | H | 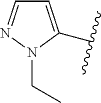 [SMILES: *c1ccnn1CC] | F | H |
| 1198 | 4198 | 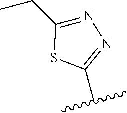 [SMILES: *c1nnc(CC)s1] | H | 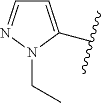 [SMILES: *c1ccnn1CC] | F | H |
| 1199 | 4199 | 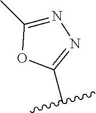 [SMILES: *c1nnc(C)o1] | H | 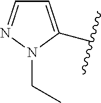 [SMILES: *c1ccnn1CC] | F | H |
| 1200 | 4200 | 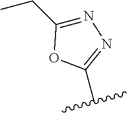 [SMILES: *c1nnc(CC)o1] | H | 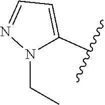 [SMILES: *c1ccnn1CC] | F | H |
| 1201 | 4201 | 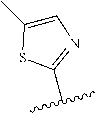 [SMILES: *c1ncc(C)s1] | CH₃ | 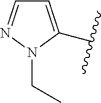 [SMILES: *c1ccnn1CC] | F | H |
| 1202 | 4202 | 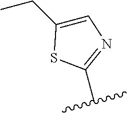 [SMILES: *c1ncc(CC)s1] | CH₃ | 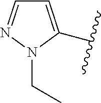 [SMILES: *c1ccnn1CC] | F | H |
| 1203 | 4203 | 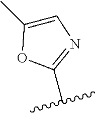 [SMILES: *c1ncc(C)o1] | CH₃ | 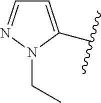 [SMILES: *c1ccnn1CC] | F | H |
| 1204 | 4204 | 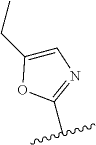 [SMILES: *c1ncc(CC)o1] | CH₃ | 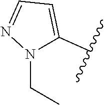 [SMILES: *c1ccnn1CC] | F | H |
| 1205 | 4205 | 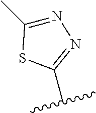 [SMILES: *c1nnc(C)s1] | CH₃ | 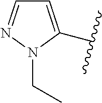 [SMILES: *c1ccnn1CC] | F | H |
| 1206 | 4206 | 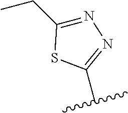 [SMILES: *c1nnc(CC)s1] | CH₃ | 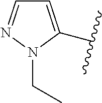 [SMILES: *c1ccnn1CC] | F | H |
| 1207 | 4207 | 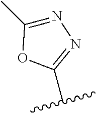 [SMILES: *c1nnc(C)o1] | CH₃ | 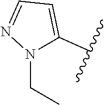 [SMILES: *c1ccnn1CC] | F | H |
| 1208 | 4208 | 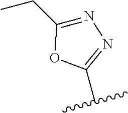 [SMILES: *c1nnc(CC)o1] | CH₃ | 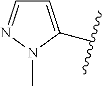 [SMILES: *c1ccnn1C] | F | H |
| 1209 | 4209 | 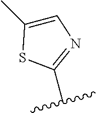 [SMILES: *c1ncc(C)s1] | H | 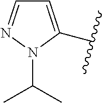 [SMILES: *c1ccnn1C(C)C] | F | H |
| 1210 | 4210 | 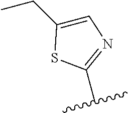 [SMILES: *c1ncc(CC)s1] | H | 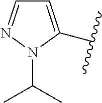 [SMILES: *c1ccnn1C(C)C] | F | H |
| 1211 | 4211 | 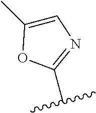 [SMILES: *c1ncc(C)o1] | H | 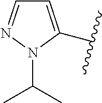 [SMILES: *c1ccnn1C(C)C] | F | H |
| 1212 | 4212 | 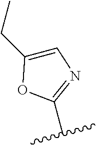 [SMILES: *c1ncc(CC)o1] | H | 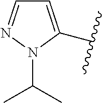 [SMILES: *c1ccnn1C(C)C] | F | H |
| 1213 | 4213 | 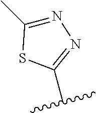 [SMILES: *c1nnc(C)s1] | H | 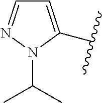 [SMILES: *c1ccnn1C(C)C] | F | H |
| 1214 | 4214 | 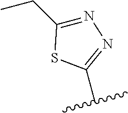 [SMILES: *c1nnc(CC)s1] | H | 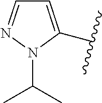 [SMILES: *c1ccnn1C(C)C] | F | H |
| 1215 | 4215 | 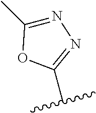 [SMILES: *c1nnc(C)o1] | H | 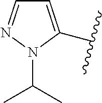 [SMILES: *c1ccnn1C(C)C] | F | H |
| 1216 | 4216 | 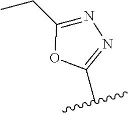 [SMILES: *c1nnc(CC)o1] | H | 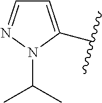 [SMILES: *c1ccnn1C(C)C] | F | H |
| 1217 | 4217 | 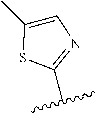 [SMILES: *c1ncc(C)s1] | CH₃ | 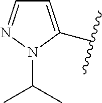 [SMILES: *c1ccnn1C(C)C] | F | H |
| 1218 | 4218 | 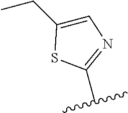 [SMILES: *c1ncc(CC)s1] | CH₃ | 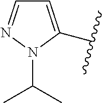 [SMILES: *c1ccnn1C(C)C] | F | H |
| 1219 | 4219 | 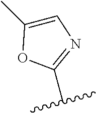 [SMILES: *c1ncc(C)o1] | CH₃ | 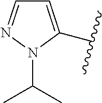 [SMILES: *c1ccnn1C(C)C] | F | H |
| 1220 | 4220 | 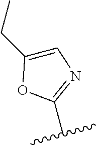 [SMILES: *c1ncc(CC)o1] | CH₃ | 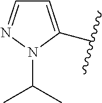 [SMILES: *c1ccnn1C(C)C] | F | H |
| 1221 | 4221 | 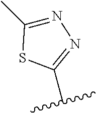 [SMILES: *c1nnc(C)s1] | CH₃ | 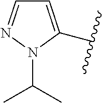 [SMILES: *c1ccnn1C(C)C] | F | H |
| 1222 | 4222 | 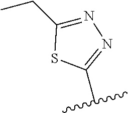 [SMILES: *c1nnc(CC)s1] | CH₃ | 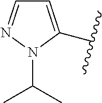 [SMILES: *c1ccnn1C(C)C] | F | H |
| 1223 | 4223 | 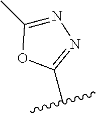 [SMILES: *c1nnc(C)o1] | CH₃ | 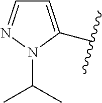 [SMILES: *c1ccnn1C(C)C] | F | H |
| 1224 | 4224 | 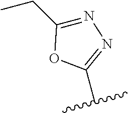 [SMILES: *c1nnc(CC)o1] | CH₃ | 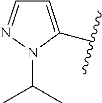 [SMILES: *c1ccnn1C(C)C] | F | H |
| 1225 | 4225 | 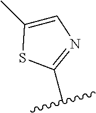 [SMILES: *c1ncc(C)s1] | H | 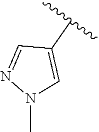 [SMILES: *c1cnn(C)c1] | F | H |
| 1226 | 4226 | 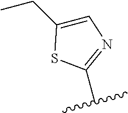 [SMILES: *c1ncc(CC)s1] | H | 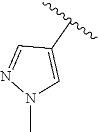 [SMILES: *c1cnn(C)c1] | F | H |
| 1227 | 4227 | 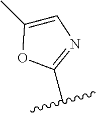 [SMILES: *c1ncc(C)o1] | H | 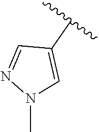 [SMILES: *c1cnn(C)c1] | F | H |
| 1228 | 4228 | 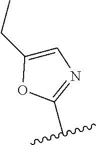 [SMILES: *c1ncc(CC)o1] | H | 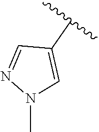 [SMILES: *c1cnn(C)c1] | F | H |
| 1229 | 4229 | 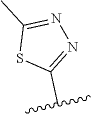 [SMILES: *c1nnc(C)s1] | H | 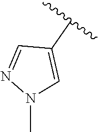 [SMILES: *c1cnn(C)c1] | F | H |
| 1230 | 4230 | 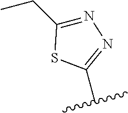 [SMILES: *c1nnc(CC)s1] | H | 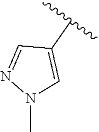 [SMILES: *c1cnn(C)c1] | F | H |
| 1231 | 4231 | 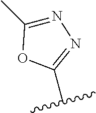 [SMILES: *c1nnc(C)o1] | H | 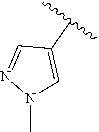 [SMILES: *c1cnn(C)c1] | F | H |
| 1232 | 4232 | 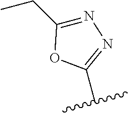 [SMILES: *c1nnc(CC)o1] | H | 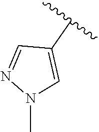 [SMILES: *c1cnn(C)c1] | F | H |
| 1233 | 4233 | 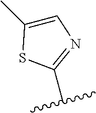 [SMILES: *c1ncc(C)s1] | CH₃ | 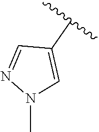 [SMILES: *c1cnn(C)c1] | F | H |
| 1234 | 4234 | 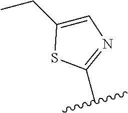 [SMILES: *c1ncc(CC)s1] | CH₃ | 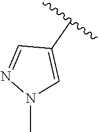 [SMILES: *c1cnn(C)c1] | F | H |
| 1235 | 4235 | 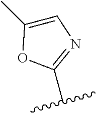 [SMILES: *c1ncc(C)o1] | CH₃ | 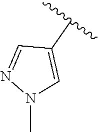 [SMILES: *c1cnn(C)c1] | F | H |
| 1236 | 4236 | 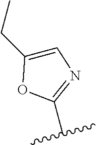 [SMILES: *c1ncc(CC)o1] | CH₃ | 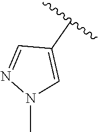 [SMILES: *c1cnn(C)c1] | F | H |
| 1237 | 4237 | 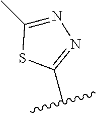 [SMILES: *c1nnc(C)s1] | CH₃ | 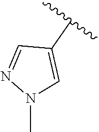 [SMILES: *c1cnn(C)c1] | F | H |
| 1238 | 4238 | 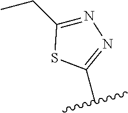 [SMILES: *c1nnc(CC)s1] | CH₃ | 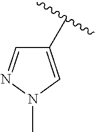 [SMILES: *c1cnn(C)c1] | F | H |
| 1239 | 4239 | 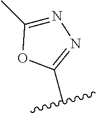 [SMILES: *c1nnc(C)o1] | CH₃ | 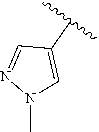 [SMILES: *c1cnn(C)c1] | F | H |
| 1240 | 4240 | 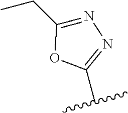 [SMILES: *c1nnc(CC)o1] | CH₃ | 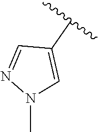 [SMILES: *c1cnn(C)c1] | F | H |
| 1241 | 4241 | 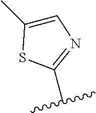 [SMILES: *c1ncc(C)s1] | H | 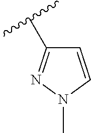 [SMILES: *c1ccn(C)n1] | F | H |
| 1242 | 4242 | 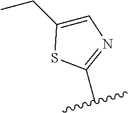 [SMILES: *c1ncc(CC)s1] | H | 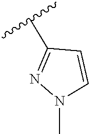 [SMILES: *c1ccn(C)n1] | F | H |
| 1243 | 4243 | 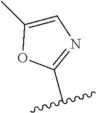 [SMILES: *c1ncc(C)o1] | H | 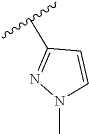 [SMILES: *c1ccn(C)n1] | F | H |
| 1244 | 4244 | 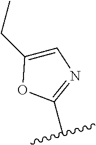 [SMILES: *c1ncc(CC)o1] | H | 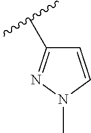 [SMILES: *c1ccn(C)n1] | F | H |
| 1245 | 4245 | 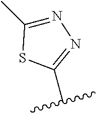 [SMILES: *c1nnc(C)s1] | H | 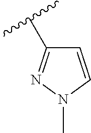 [SMILES: *c1ccn(C)n1] | F | H |
| 1246 | 4246 | 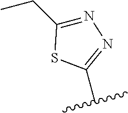 [SMILES: *c1nnc(CC)s1] | H | 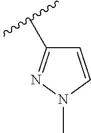 [SMILES: *c1ccn(C)n1] | F | H |
| 1247 | 4247 | 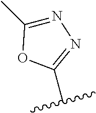 [SMILES: *c1nnc(C)o1] | H | 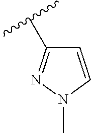 [SMILES: *c1ccn(C)n1] | F | H |
| 1248 | 4248 | 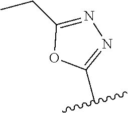 [SMILES: *c1nnc(CC)o1] | H | 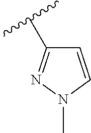 [SMILES: *c1ccn(C)n1] | F | H |
| 1249 | 4249 | 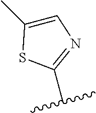 [SMILES: *c1ncc(C)s1] | CH₃ | 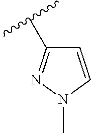 [SMILES: *c1ccn(C)n1] | F | H |
| 1250 | 4250 | 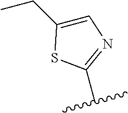 [SMILES: *c1ncc(CC)s1] | CH₃ | 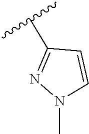 [SMILES: *c1ccn(C)n1] | F | H |
| 1251 | 4251 | 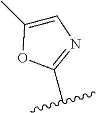 [SMILES: *c1ncc(C)o1] | CH₃ | 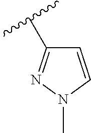 [SMILES: *c1ccn(C)n1] | F | H |
| 1252 | 4252 | 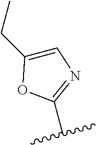 [SMILES: *c1ncc(CC)o1] | CH₃ | 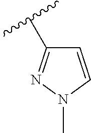 [SMILES: *c1ccn(C)n1] | F | H |
| 1253 | 4253 | 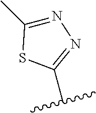 [SMILES: *c1nnc(C)s1] | CH₃ | 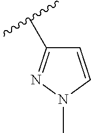 [SMILES: *c1ccn(C)n1] | F | H |
| 1254 | 4254 | 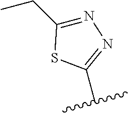 [SMILES: *c1nnc(CC)s1] | CH₃ | 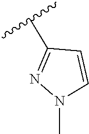 [SMILES: *c1ccn(C)n1] | F | H |
| 1255 | 4255 | 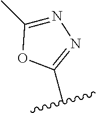 [SMILES: *c1nnc(C)o1] | CH₃ | 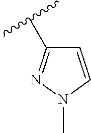 [SMILES: *c1ccn(C)n1] | F | H |
| 1256 | 4256 | 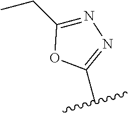 [SMILES: *c1nnc(CC)o1] | CH₃ | 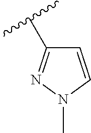 [SMILES: *c1ccn(C)n1] | F | H |
| 1257 | 4257 | 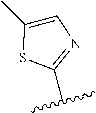 [SMILES: *c1ncc(C)s1] | H | 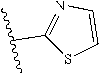 [SMILES: *c1nccs1] | F | H |
| 1258 | 4258 | 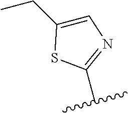 [SMILES: *c1ncc(CC)s1] | H | 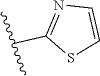 [SMILES: *c1nccs1] | F | H |
| 1259 | 4259 | 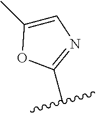 [SMILES: *c1ncc(C)o1] | H | 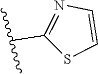 [SMILES: *c1nccs1] | F | H |
| 1260 | 4260 | 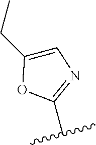 [SMILES: *c1ncc(CC)o1] | H | 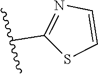 [SMILES: *c1nccs1] | F | H |
| 1261 | 4261 | 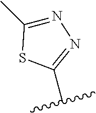 [SMILES: *c1nnc(C)s1] | H | 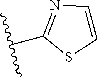 [SMILES: *c1nccs1] | F | H |
| 1262 | 4262 | 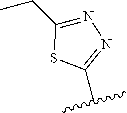 [SMILES: *c1nnc(CC)s1] | H | 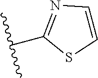 [SMILES: *c1nccs1] | F | H |
| 1263 | 4263 | 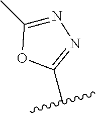 [SMILES: *c1nnc(C)o1] | H | 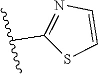 [SMILES: *c1nccs1] | F | H |
| 1264 | 4264 | 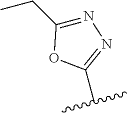 [SMILES: *c1nnc(CC)o1] | H | 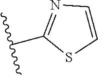 [SMILES: *c1nccs1] | F | H |
| 1265 | 4265 | 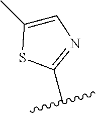 [SMILES: *c1ncc(C)s1] | CH₃ | 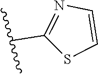 [SMILES: *c1nccs1] | F | H |
| 1266 | 4266 | 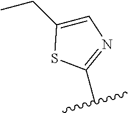 [SMILES: *c1ncc(CC)s1] | CH₃ | 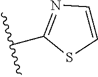 [SMILES: *c1nccs1] | F | H |
| 1267 | 4267 | 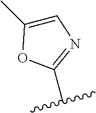 [SMILES: *c1ncc(C)o1] | CH₃ | 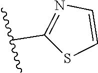 [SMILES: *c1nccs1] | F | H |
| 1268 | 4268 | 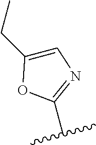 [SMILES: *c1ncc(CC)o1] | CH₃ | 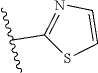 [SMILES: *c1nccs1] | F | H |
| 1269 | 4269 | 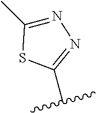 [SMILES: *c1nnc(C)s1] | CH₃ | 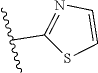 [SMILES: *c1nccs1] | F | H |
| 1270 | 4270 | 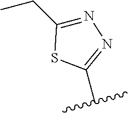 [SMILES: *c1nnc(CC)s1] | CH₃ | 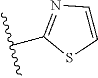 [SMILES: *c1nccs1] | F | H |
| 1271 | 4271 | 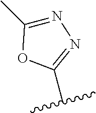 [SMILES: *c1nnc(C)o1] | CH₃ | 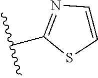 [SMILES: *c1nccs1] | F | H |
| 1272 | 4272 | 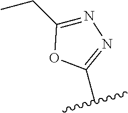 [SMILES: *c1nnc(CC)o1] | CH₃ | 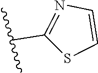 [SMILES: *c1nccs1] | F | H |
| 1289 | 4289 | 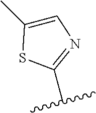 [SMILES: *c1ncc(C)s1] | H | 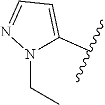 [SMILES: *c1ccnn1CC] | H | Cl |
| 1290 | 4290 | 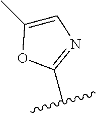 [SMILES: *c1ncc(C)o1] | H | 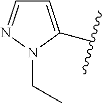 [SMILES: *c1ccnn1CC] | H | Cl |
| 1291 | 4291 | 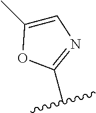 [SMILES: *c1ncc(C)o1] | H | 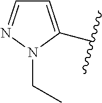 [SMILES: *c1ccnn1CC] | H | Cl |
| 1292 | 4292 | 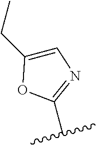 [SMILES: *c1ncc(CC)o1] | H | 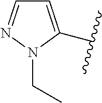 [SMILES: *c1ccnn1CC] | H | Cl |
| 1293 | 4293 | 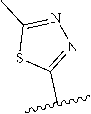 [SMILES: *c1nnc(C)s1] | H | 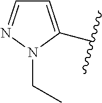 [SMILES: *c1ccnn1CC] | H | Cl |
| 1294 | 4294 | 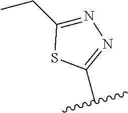 [SMILES: *c1nnc(CC)s1] | H | 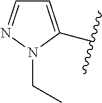 [SMILES: *c1ccnn1CC] | H | Cl |
| 1295 | 4295 | 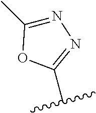 [SMILES: *c1nnc(C)o1] | H | 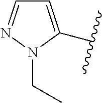 [SMILES: *c1ccnn1CC] | H | Cl |
| 1296 | 4296 | 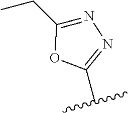 [SMILES: *c1nnc(CC)o1] | H | 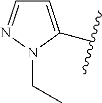 [SMILES: *c1ccnn1CC] | H | Cl |
| 1297 | 4297 | 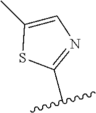 [SMILES: *c1ncc(C)s1] | CH₃ | 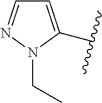 [SMILES: *c1ccnn1CC] | H | Cl |
| 1298 | 4298 | 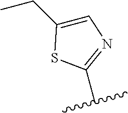 [SMILES: *c1ncc(CC)s1] | CH₃ | 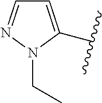 [SMILES: *c1ccnn1CC] | H | Cl |
| 1299 | 4299 | 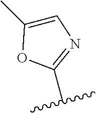 [SMILES: *c1ncc(C)o1] | CH₃ | 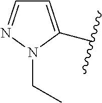 [SMILES: *c1ccnn1CC] | H | Cl |
| 1300 | 4300 | 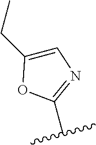 [SMILES: *c1ncc(CC)o1] | CH₃ | 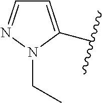 [SMILES: *c1ccnn1CC] | H | Cl |
| 1301 | 4301 | 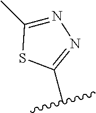 [SMILES: *c1nnc(C)s1] | CH₃ | 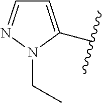 [SMILES: *c1ccnn1CC] | H | Cl |
| 1302 | 4302 | 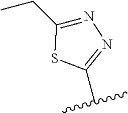 [SMILES: *c1nnc(CC)s1] | CH₃ | 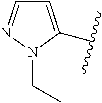 [SMILES: *c1ccnn1CC] | H | Cl |
| 1303 | 4303 | 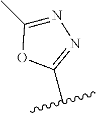 [SMILES: *c1nnc(C)o1] | CH₃ | 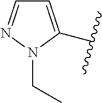 [SMILES: *c1ccnn1CC] | H | Cl |
| 1304 | 4304 | 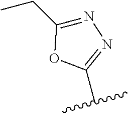 [SMILES: *c1nnc(CC)o1] | CH₃ | 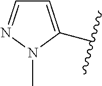 [SMILES: *c1ccnn1C] | H | Cl |
| 1305 | 4305 | 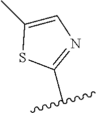 [SMILES: *c1ncc(C)s1] | H | 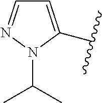 [SMILES: *c1ccnn1C(C)C] | H | Cl |
| 1306 | 4306 | 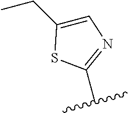 [SMILES: *c1ncc(CC)s1] | H | 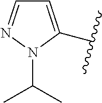 [SMILES: *c1ccnn1C(C)C] | H | Cl |
| 1307 | 4307 | 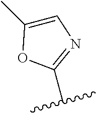 [SMILES: *c1ncc(C)o1] | H | 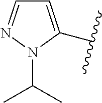 [SMILES: *c1ccnn1C(C)C] | H | Cl |
| 1308 | 4308 | 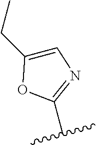 [SMILES: *c1ncc(CC)o1] | H | 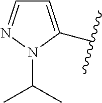 [SMILES: *c1ccnn1C(C)C] | H | Cl |
| 1309 | 4309 | 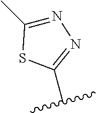 [SMILES: *c1nnc(C)s1] | H | 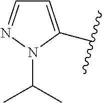 [SMILES: *c1ccnn1C(C)C] | H | Cl |
| 1310 | 4310 | 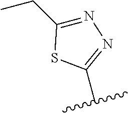 [SMILES: *c1nnc(CC)s1] | H | 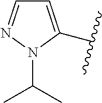 [SMILES: *c1ccnn1C(C)C] | H | Cl |
| 1311 | 4311 | 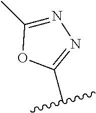 [SMILES: *c1nnc(C)o1] | H | 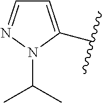 [SMILES: *c1ccnn1C(C)C] | H | Cl |
| 1312 | 4312 | 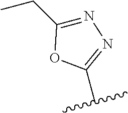 [SMILES: *c1nnc(CC)o1] | H | 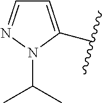 [SMILES: *c1ccnn1C(C)C] | H | Cl |
| 1313 | 4313 | 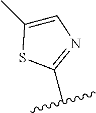 [SMILES: *c1ncc(C)s1] | CH₃ | 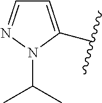 [SMILES: *c1ccnn1C(C)C] | H | Cl |
| 1314 | 4314 | 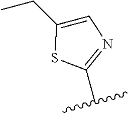 [SMILES: *c1ncc(CC)s1] | CH₃ | 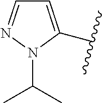 [SMILES: *c1ccnn1C(C)C] | H | Cl |
| 1315 | 4315 | 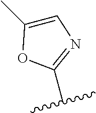 [SMILES: *c1ncc(C)o1] | CH₃ | 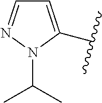 [SMILES: *c1ccnn1C(C)C] | H | Cl |
| 1316 | 4316 | 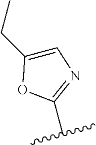 [SMILES: *c1ncc(CC)o1] | CH₃ | 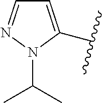 [SMILES: *c1ccnn1C(C)C] | H | Cl |
| 1317 | 4317 | 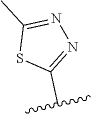 [SMILES: *c1nnc(C)s1] | CH₃ | 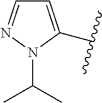 [SMILES: *c1ccnn1C(C)C] | H | Cl |
| 1318 | 4318 | 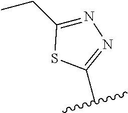 [SMILES: *c1nnc(CC)s1] | CH₃ | 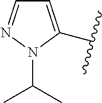 [SMILES: *c1ccnn1C(C)C] | H | Cl |
| 1319 | 4319 | 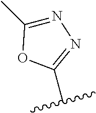 [SMILES: *c1nnc(C)o1] | CH₃ | 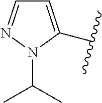 [SMILES: *c1ccnn1C(C)C] | H | Cl |
| 1320 | 4320 | 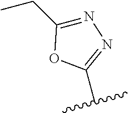 [SMILES: *c1nnc(CC)o1] | CH₃ | 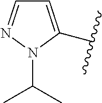 [SMILES: *c1ccnn1C(C)C] | H | Cl |
| 1321 | 4321 | 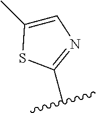 [SMILES: *c1ncc(C)s1] | H | 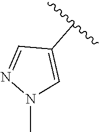 [SMILES: *c1cnn(C)c1] | H | Cl |
| 1322 | 4322 | 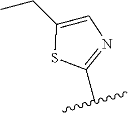 [SMILES: *c1ncc(CC)s1] | H | 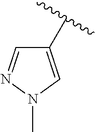 [SMILES: *c1cnn(C)c1] | H | Cl |
| 1323 | 4323 | 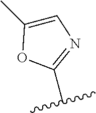 [SMILES: *c1ncc(C)o1] | H | 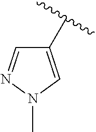 [SMILES: *c1cnn(C)c1] | H | Cl |
| 1324 | 4324 | 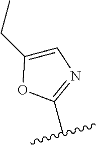 [SMILES: *c1ncc(CC)o1] | H | 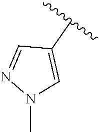 [SMILES: *c1cnn(C)c1] | H | Cl |
| 1325 | 4325 | 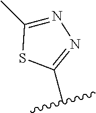 [SMILES: *c1nnc(C)s1] | H | 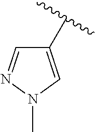 [SMILES: *c1cnn(C)c1] | H | Cl |
| 1326 | 4326 | 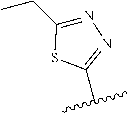 [SMILES: *c1nnc(CC)s1] | H | 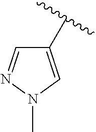 [SMILES: *c1cnn(C)c1] | H | Cl |
| 1327 | 4327 | 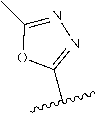 [SMILES: *c1nnc(C)o1] | H | 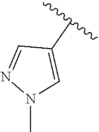 [SMILES: *c1cnn(C)c1] | H | Cl |
| 1328 | 4328 | 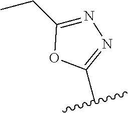 [SMILES: *c1nnc(CC)o1] | H | 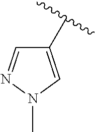 [SMILES: *c1cnn(C)c1] | H | Cl |
| 1329 | 4329 | 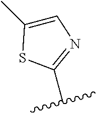 [SMILES: *c1ncc(C)s1] | CH₃ | 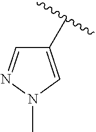 [SMILES: *c1cnn(C)c1] | H | Cl |
| 1330 | 4330 | 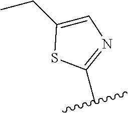 [SMILES: *c1ncc(CC)s1] | CH₃ | 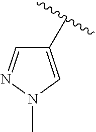 [SMILES: *c1cnn(C)c1] | H | Cl |
| 1331 | 4331 | 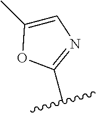 [SMILES: *c1ncc(C)o1] | CH₃ | 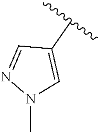 [SMILES: *c1cnn(C)c1] | H | Cl |
| 1332 | 4332 | 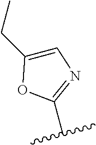 [SMILES: *c1ncc(CC)o1] | CH₃ | 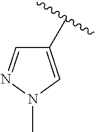 [SMILES: *c1cnn(C)c1] | H | Cl |
| 1333 | 4333 | 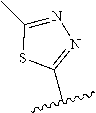 [SMILES: *c1nnc(C)s1] | CH₃ | 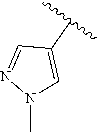 [SMILES: *c1cnn(C)c1] | H | Cl |
| 1334 | 4334 | 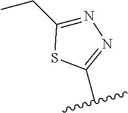 [SMILES: *c1nnc(CC)s1] | CH₃ | 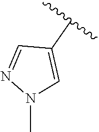 [SMILES: *c1cnn(C)c1] | H | Cl |
| 1335 | 4335 | 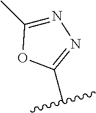 [SMILES: *c1nnc(C)o1] | CH₃ | 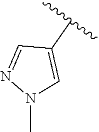 [SMILES: *c1cnn(C)c1] | H | Cl |
| 1336 | 4336 | 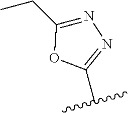 [SMILES: *c1nnc(CC)o1] | CH₃ | 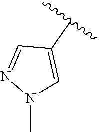 [SMILES: *c1cnn(C)c1] | H | Cl |
| 1337 | 4337 | 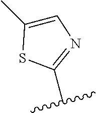 [SMILES: *c1ncc(C)s1] | H | 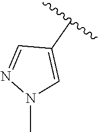 [SMILES: *c1cnn(C)c1] | H | Cl |
| 1338 | 4338 | 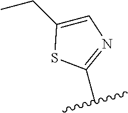 [SMILES: *c1ncc(CC)s1] | H | 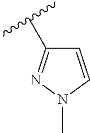 [SMILES: *c1ccn(C)n1] | H | Cl |
| 1339 | 4339 | 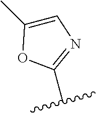 [SMILES: *c1ncc(C)o1] | H | 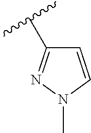 [SMILES: *c1ccn(C)n1] | H | Cl |
| 1340 | 4340 | 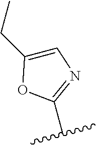 [SMILES: *c1ncc(CC)o1] | H | 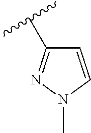 [SMILES: *c1ccn(C)n1] | H | Cl |
| 1341 | 4341 | 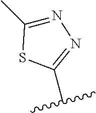 [SMILES: *c1nnc(C)s1] | H | 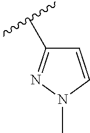 [SMILES: *c1ccn(C)n1] | H | Cl |
| 1342 | 4342 | 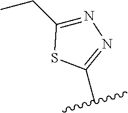 [SMILES: *c1nnc(CC)s1] | H | 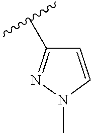 [SMILES: *c1ccn(C)n1] | H | Cl |
| 1343 | 4343 | 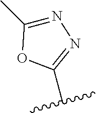 [SMILES: *c1nnc(C)o1] | H | 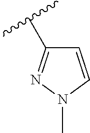 [SMILES: *c1ccn(C)n1] | H | Cl |
| 1344 | 4344 | 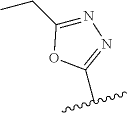 [SMILES: *c1nnc(CC)o1] | H | 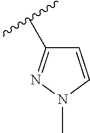 [SMILES: *c1ccn(C)n1] | H | Cl |
| 1345 | 4345 | 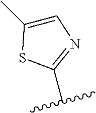 [SMILES: *c1ncc(C)s1] | CH₃ | 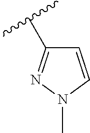 [SMILES: *c1ccn(C)n1] | H | Cl |
| 1346 | 4346 | 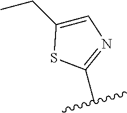 [SMILES: *c1ncc(CC)s1] | CH₃ | 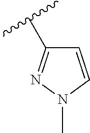 [SMILES: *c1ccn(C)n1] | H | Cl |
| 1347 | 4347 | 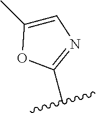 [SMILES: *c1ncc(C)o1] | CH₃ | 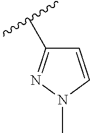 [SMILES: *c1ccn(C)n1] | H | Cl |
| 1348 | 4348 | 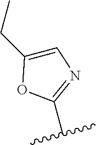 [SMILES: *c1ncc(CC)o1] | CH₃ | 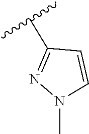 [SMILES: *c1ccn(C)n1] | H | Cl |
| 1349 | 4349 | 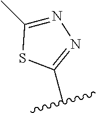 [SMILES: *c1nnc(C)s1] | CH₃ | 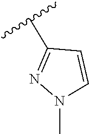 [SMILES: *c1ccn(C)n1] | H | Cl |
| 1350 | 4350 | 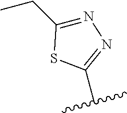 [SMILES: *c1nnc(CC)s1] | CH₃ | 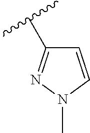 [SMILES: *c1ccn(C)n1] | H | Cl |
| 1351 | 4351 | 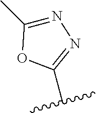 [SMILES: *c1nnc(C)o1] | CH₃ | 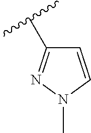 [SMILES: *c1ccn(C)n1] | H | Cl |
| 1352 | 4352 | 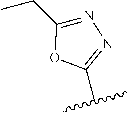 [SMILES: *c1nnc(CC)o1] | CH₃ | 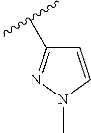 [SMILES: *c1ccn(C)n1] | H | Cl |
| 1353 | 4353 | 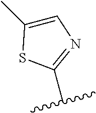 [SMILES: *c1ncc(C)s1] | H | 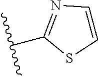 [SMILES: *c1nccs1] | H | Cl |
| 1354 | 4354 | 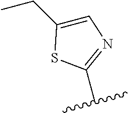 [SMILES: *c1ncc(CC)s1] | H | 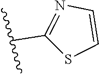 [SMILES: *c1nccs1] | H | Cl |
| 1355 | 4355 | 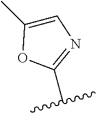 [SMILES: *c1ncc(C)o1] | H | 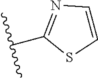 [SMILES: *c1nccs1] | H | Cl |
| 1356 | 4356 | 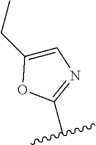 [SMILES: *c1ncc(CC)o1] | H | 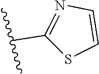 [SMILES: *c1nccs1] | H | Cl |
| 1357 | 4357 | 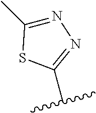 [SMILES: *c1nnc(C)s1] | H | 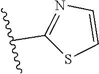 [SMILES: *c1nccs1] | H | Cl |
| 1358 | 4358 | 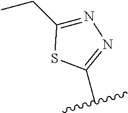 [SMILES: *c1nnc(CC)s1] | H | 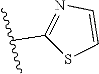 [SMILES: *c1nccs1] | H | Cl |
| 1359 | 4359 | 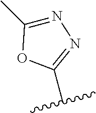 [SMILES: *c1nnc(C)o1] | H | 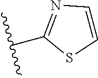 [SMILES: *c1nccs1] | H | Cl |
| 1360 | 4360 | 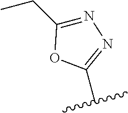 [SMILES: *c1nnc(CC)o1] | H | 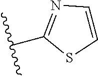 [SMILES: *c1nccs1] | H | Cl |
| 1361 | 4361 | 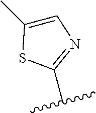 [SMILES: *c1ncc(C)s1] | CH₃ | 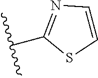 [SMILES: *c1nccs1] | H | Cl |
| 1362 | 4362 | 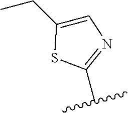 [SMILES: *c1ncc(CC)s1] | CH₃ | 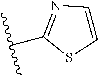 [SMILES: *c1nccs1] | H | Cl |
| 1363 | 4363 | 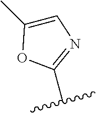 [SMILES: *c1ncc(C)o1] | CH₃ | 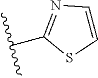 [SMILES: *c1nccs1] | H | Cl |
| 1364 | 4364 | 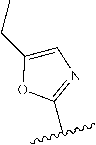 [SMILES: *c1ncc(CC)o1] | CH₃ | 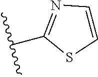 [SMILES: *c1nccs1] | H | Cl |
| 1365 | 4365 | 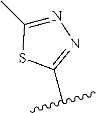 [SMILES: *c1nnc(C)s1] | CH₃ | 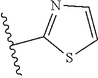 [SMILES: *c1nccs1] | H | Cl |
| 1366 | 4366 | 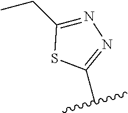 [SMILES: *c1nnc(CC)s1] | CH₃ | 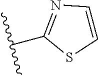 [SMILES: *c1nccs1] | H | Cl |
| 1367 | 4367 | 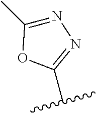 [SMILES: *c1nnc(C)o1] | CH₃ | 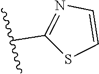 [SMILES: *c1nccs1] | H | Cl |
| 1368 | 4368 | 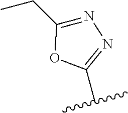 [SMILES: *c1nnc(CC)o1] | CH₃ | 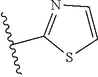 [SMILES: *c1nccs1] | H | Cl |
| 1385 | 4385 | 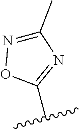 [SMILES: *c1nc(C)no1] | H | 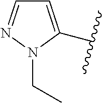 [SMILES: *c1ccnn1CC] | H | H |
| 1386 | 4386 | 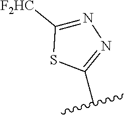 [SMILES: *c1nnc(C(F)F)s1] | H | 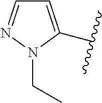 [SMILES: *c1ccnn1CC] | H | H |
| 1387 | 4387 | 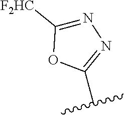 [SMILES: *c1nnc(C(F)F)o1] | H | 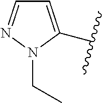 [SMILES: *c1ccnn1CC] | H | H |
| 1388 | 4388 | 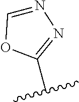 [SMILES: *c1nnco1] | H | 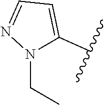 [SMILES: *c1ccnn1CC] | H | H |
| 1389 | 4389 | 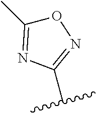 [SMILES: *c1noc(C)n1] | H | 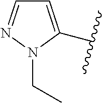 [SMILES: *c1ccnn1CC] | H | H |
| 1390 | 4390 | 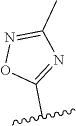 [SMILES: *c1nc(C)no1] | CH₃ | 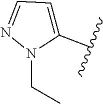 [SMILES: *c1ccnn1CC] | H | H |
| 1391 | 4391 | 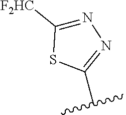 [SMILES: *c1nnc(C(F)F)s1] | CH₃ | 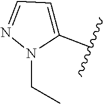 [SMILES: *c1ccnn1CC] | H | H |
| 1392 | 4392 | 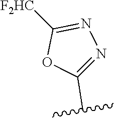 [SMILES: *c1nnc(C(F)F)o1] | CH₃ | 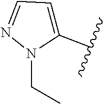 [SMILES: *c1ccnn1CC] | H | H |
| 1393 | 4393 | 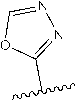 [SMILES: *c1nnco1] | CH₃ | 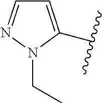 [SMILES: *c1ccnn1CC] | H | H |
| 1394 | 4394 | 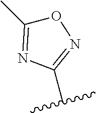 [SMILES: *c1noc(C)n1] | CH₃ | 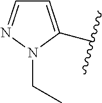 [SMILES: *c1ccnn1CC] | H | H |
| 1395 | 4395 | 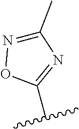 [SMILES: *c1nc(C)no1] | H | 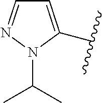 [SMILES: *c1ccnn1C(C)C] | H | H |
| 1396 | 4396 | 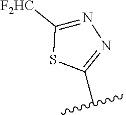 [SMILES: *c1nnc(C(F)F)s1] | H | 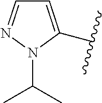 [SMILES: *c1ccnn1C(C)C] | H | H |
| 1397 | 4397 | 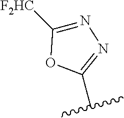 [SMILES: *c1nnc(C(F)F)o1] | H | 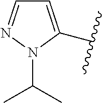 [SMILES: *c1ccnn1C(C)C] | H | H |
| 1398 | 4398 | 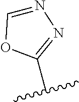 [SMILES: *c1nnco1] | H | 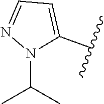 [SMILES: *c1ccnn1C(C)C] | H | H |
| 1399 | 4399 | 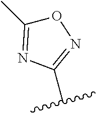 [SMILES: *c1noc(C)n1] | H | 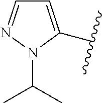 [SMILES: *c1ccnn1C(C)C] | H | H |
| 1400 | 4400 | 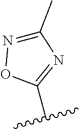 [SMILES: *c1nc(C)no1] | CH₃ | 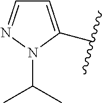 [SMILES: *c1ccnn1C(C)C] | H | H |
| 1401 | 4401 | 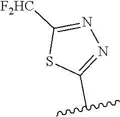 [SMILES: *c1nnc(C(F)F)s1] | CH₃ | 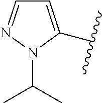 [SMILES: *c1ccnn1C(C)C] | H | H |
| 1402 | 4402 | 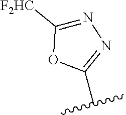 [SMILES: *c1nnc(C(F)F)o1] | CH₃ | 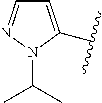 [SMILES: *c1ccnn1C(C)C] | H | H |
| 1403 | 4403 | 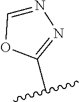 [SMILES: *c1nnco1] | CH₃ | 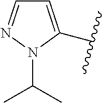 [SMILES: *c1ccnn1C(C)C] | H | H |
| 1404 | 4404 | 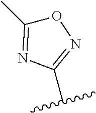 [SMILES: *c1noc(C)n1] | CH₃ | 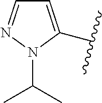 [SMILES: *c1ccnn1C(C)C] | H | H |
| 1405 | 4405 | 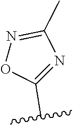 [SMILES: *c1nc(C)no1] | H | 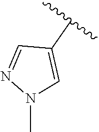 [SMILES: *c1cnn(C)c1] | H | H |
| 1406 | 4406 | 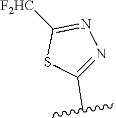 [SMILES: *c1nnc(C(F)F)s1] | H | 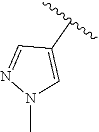 [SMILES: *c1cnn(C)c1] | H | H |
| 1407 | 4407 | 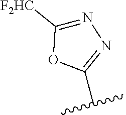 [SMILES: *c1nnc(C(F)F)o1] | H | 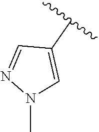 [SMILES: *c1cnn(C)c1] | H | H |
| 1408 | 4408 | 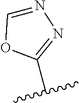 [SMILES: *c1nnco1] | H | 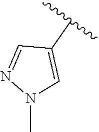 [SMILES: *c1cnn(C)c1] | H | H |
| 1409 | 4409 | 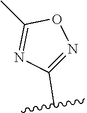 [SMILES: *c1noc(C)n1] | H | 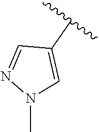 [SMILES: *c1cnn(C)c1] | H | H |
| 1410 | 4410 | 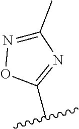 [SMILES: *c1nc(C)no1] | CH₃ | 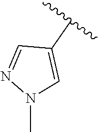 [SMILES: *c1cnn(C)c1] | H | H |
| 1411 | 4411 | 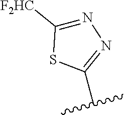 [SMILES: *c1nnc(C(F)F)s1] | CH₃ | 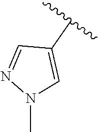 [SMILES: *c1cnn(C)c1] | H | H |
| 1412 | 4412 | 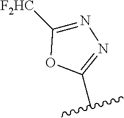 [SMILES: *c1nnc(C(F)F)o1] | CH₃ | 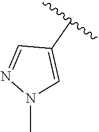 [SMILES: *c1cnn(C)c1] | H | H |
| 1413 | 4413 | 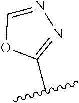 [SMILES: *c1nnco1] | CH₃ | 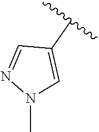 [SMILES: *c1cnn(C)c1] | H | H |
| 1414 | 4414 | 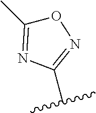 [SMILES: *c1noc(C)n1] | CH₃ | 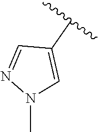 [SMILES: *c1cnn(C)c1] | H | H |
| 1415 | 4415 | 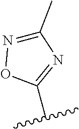 [SMILES: *c1nc(C)no1] | H | 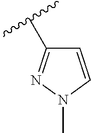 [SMILES: *c1ccn(C)n1] | H | H |
| 1416 | 4416 | 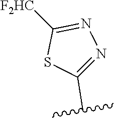 [SMILES: *c1nnc(C(F)F)s1] | H | 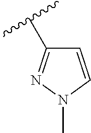 [SMILES: *c1ccn(C)n1] | H | H |
| 1417 | 4417 | 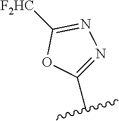 [SMILES: *c1nnc(C(F)F)o1] | H | 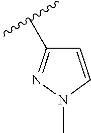 [SMILES: *c1ccn(C)n1] | H | H |
| 1418 | 4418 | 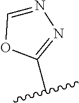 [SMILES: *c1nnco1] | H | 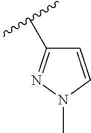 [SMILES: *c1ccn(C)n1] | H | H |
| 1419 | 4419 | 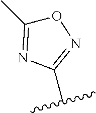 [SMILES: *c1noc(C)n1] | H | 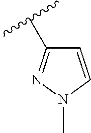 [SMILES: *c1ccn(C)n1] | H | H |
| 1420 | 4420 | 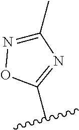 [SMILES: *c1nc(C)no1] | CH₃ | 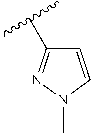 [SMILES: *c1ccn(C)n1] | H | H |
| 1421 | 4421 | 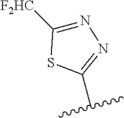 [SMILES: *c1nnc(C(F)F)s1] | CH₃ | 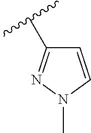 [SMILES: *c1ccn(C)n1] | H | H |
| 1422 | 4422 | 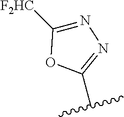 [SMILES: *c1nnc(C(F)F)o1] | CH₃ | 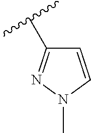 [SMILES: *c1ccn(C)n1] | H | H |
| 1423 | 4423 | 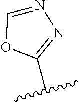 [SMILES: *c1nnco1] | CH₃ | 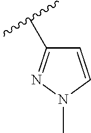 [SMILES: *c1ccn(C)n1] | H | H |
| 1424 | 4424 | 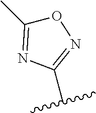 [SMILES: *c1noc(C)n1] | CH₃ | 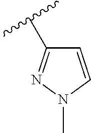 [SMILES: *c1ccn(C)n1] | H | H |
| 1425 | 4425 | 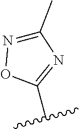 [SMILES: *c1nc(C)no1] | H | 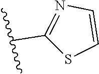 [SMILES: *c1nccs1] | H | H |
| 1426 | 4426 | 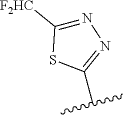 [SMILES: *c1nnc(C(F)F)s1] | H | 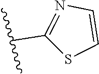 [SMILES: *c1nccs1] | H | H |
| 1427 | 4427 | 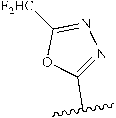 [SMILES: *c1nnc(C(F)F)o1] | H | 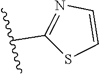 [SMILES: *c1nccs1] | H | H |
| 1428 | 4428 | 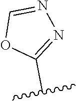 [SMILES: *c1nnco1] | H | 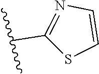 [SMILES: *c1nccs1] | H | H |
| 1429 | 4429 | 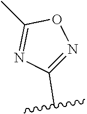 [SMILES: *c1noc(C)n1] | H | 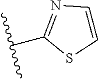 [SMILES: *c1nccs1] | H | H |
| 1430 | 4430 | 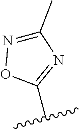 [SMILES: *c1nc(C)no1] | CH₃ | 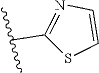 [SMILES: *c1nccs1] | H | H |
| 1431 | 4431 | 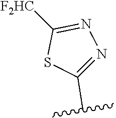 [SMILES: *c1nnc(C(F)F)s1] | CH₃ | 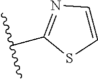 [SMILES: *c1nccs1] | H | H |
| 1432 | 4432 | 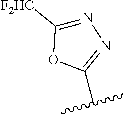 [SMILES: *c1nnc(C(F)F)o1] | CH₃ | 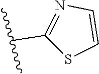 [SMILES: *c1nccs1] | H | H |
| 1433 | 4433 | 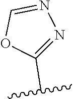 [SMILES: *c1nnco1] | CH₃ | 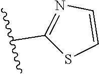 [SMILES: *c1nccs1] | H | H |
| 1434 | 4434 | 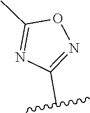 [SMILES: *c1noc(C)n1] | CH₃ | 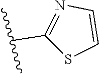 [SMILES: *c1nccs1] | H | H |
| 1435 | 4435 | 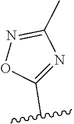 [SMILES: *c1nc(C)no1] | H | 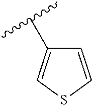 [SMILES: *c1ccsc1] | H | H |
| 1436 | 4436 | 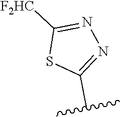 [SMILES: *c1nnc(C(F)F)s1] | H | 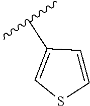 [SMILES: *c1ccsc1] | H | H |
| 1437 | 4437 | 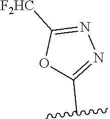 [SMILES: *c1nnc(C(F)F)o1] | H | 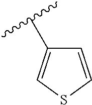 [SMILES: *c1ccsc1] | H | H |
| 1438 | 4438 | 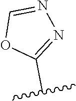 [SMILES: *c1nnco1] | H | 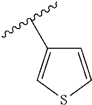 [SMILES: *c1ccsc1] | H | H |
| 1439 | 4439 | 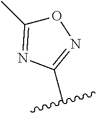 [SMILES: *c1noc(C)n1] | H | 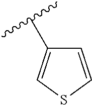 [SMILES: *c1ccsc1] | H | H |
| 1440 | 4440 | 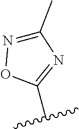 [SMILES: *c1nc(C)no1] | CH₃ | 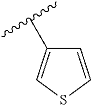 [SMILES: *c1ccsc1] | H | H |
| 1441 | 4441 | 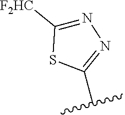 [SMILES: *c1nnc(C(F)F)s1] | CH₃ | 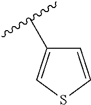 [SMILES: *c1ccsc1] | H | H |
| 1442 | 4442 | 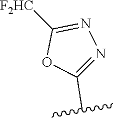 [SMILES: *c1nnc(C(F)F)o1] | CH₃ | 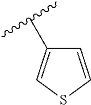 [SMILES: *c1ccsc1] | H | H |
| 1443 | 4443 | 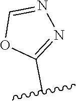 [SMILES: *c1nnco1] | CH₃ | 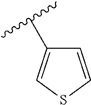 [SMILES: *c1ccsc1] | H | H |
| 1444 | 4444 | 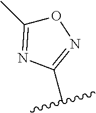 [SMILES: *c1noc(C)n1] | CH₃ | 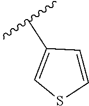 [SMILES: *c1ccsc1] | H | H |
| 1445 | 4445 | 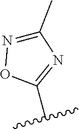 [SMILES: *c1nc(C)no1] | H | 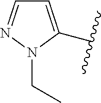 [SMILES: *c1ccnn1CC] | H | F |
| 1446 | 4446 | 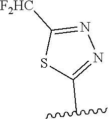 [SMILES: *c1nnc(C(F)F)s1] | H | 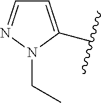 [SMILES: *c1ccnn1CC] | H | F |
| 1447 | 4447 | 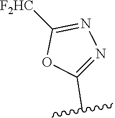 [SMILES: *c1nnc(C(F)F)o1] | H | 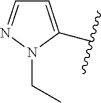 [SMILES: *c1ccnn1CC] | H | F |
| 1448 | 4448 | 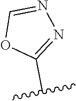 [SMILES: *c1nnco1] | H | 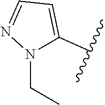 [SMILES: *c1ccnn1CC] | H | F |
| 1449 | 4449 | 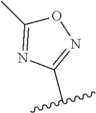 [SMILES: *c1noc(C)n1] | H | 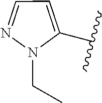 [SMILES: *c1ccnn1CC] | H | F |
| 1450 | 4450 | 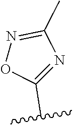 [SMILES: *c1nc(C)no1] | CH₃ | 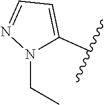 [SMILES: *c1ccnn1CC] | H | F |
| 1451 | 4451 | 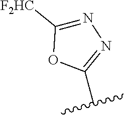 [SMILES: *c1nnc(C(F)F)o1] | CH₃ | 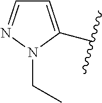 [SMILES: *c1ccnn1CC] | H | F |
| 1452 | 4452 | 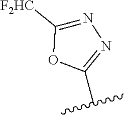 [SMILES: *c1nnc(C(F)F)o1] | CH₃ | 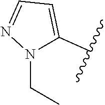 [SMILES: *c1ccnn1CC] | H | F |
| 1453 | 4453 | 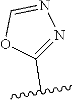 [SMILES: *c1nnco1] | CH₃ | 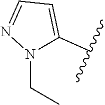 [SMILES: *c1ccnn1CC] | H | F |
| 1454 | 4454 | 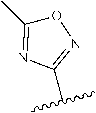 [SMILES: *c1noc(C)n1] | CH₃ | 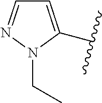 [SMILES: *c1ccnn1CC] | H | F |
| 1455 | 4455 | 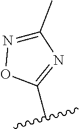 [SMILES: *c1nc(C)no1] | H | 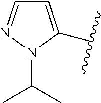 [SMILES: *c1ccnn1C(C)C] | H | F |
| 1456 | 4456 | 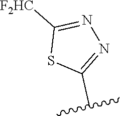 [SMILES: *c1nnc(C(F)F)s1] | H | 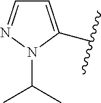 [SMILES: *c1ccnn1C(C)C] | H | F |
| 1457 | 4457 | 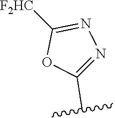 [SMILES: *c1nnc(C(F)F)o1] | H | 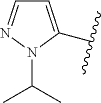 [SMILES: *c1ccnn1C(C)C] | H | F |
| 1458 | 4458 | 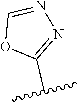 [SMILES: *c1nnco1] | H | 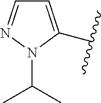 [SMILES: *c1ccnn1C(C)C] | H | F |
| 1459 | 4459 | 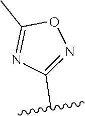 [SMILES: *c1noc(C)n1] | H | 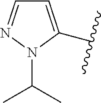 [SMILES: *c1ccnn1C(C)C] | H | F |
| 1460 | 4460 | 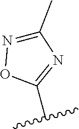 [SMILES: *c1nc(C)no1] | CH₃ | 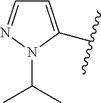 [SMILES: *c1ccnn1C(C)C] | H | F |
| 1461 | 4461 | 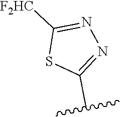 [SMILES: *c1nnc(C(F)F)s1] | CH₃ | 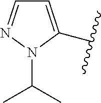 [SMILES: *c1ccnn1C(C)C] | H | F |
| 1462 | 4462 | 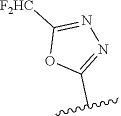 [SMILES: *c1nnc(C(F)F)o1] | CH₃ | 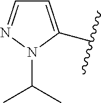 [SMILES: *c1ccnn1C(C)C] | H | F |
| 1463 | 4463 | 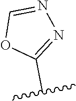 [SMILES: *c1nnco1] | CH₃ | 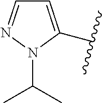 [SMILES: *c1ccnn1C(C)C] | H | F |
| 1464 | 4464 | 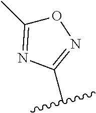 [SMILES: *c1noc(C)n1] | CH₃ | 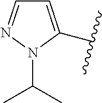 [SMILES: *c1ccnn1C(C)C] | H | F |
| 1465 | 4465 | 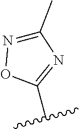 [SMILES: *c1nc(C)no1] | H | 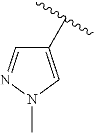 [SMILES: *c1cnn(C)c1] | H | F |
| 1466 | 4466 | 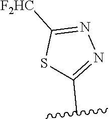 [SMILES: *c1nnc(C(F)F)s1] | H | 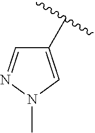 [SMILES: *c1cnn(C)c1] | H | F |
| 1467 | 4467 | 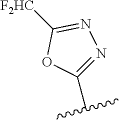 [SMILES: *c1nnc(C(F)F)o1] | H | 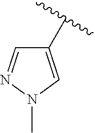 [SMILES: *c1cnn(C)c1] | H | F |
| 1468 | 4468 | 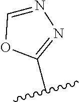 [SMILES: *c1nnco1] | H | 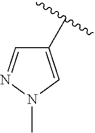 [SMILES: *c1cnn(C)c1] | H | F |
| 1469 | 4469 | 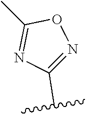 [SMILES: *c1noc(C)n1] | H | 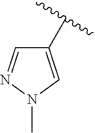 [SMILES: *c1cnn(C)c1] | H | F |
| 1470 | 4470 | 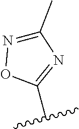 [SMILES: *c1nc(C)no1] | CH₃ | 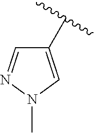 [SMILES: *c1cnn(C)c1] | H | F |
| 1471 | 4471 | 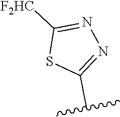 [SMILES: *c1nnc(C(F)F)s1] | CH₃ | 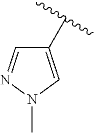 [SMILES: *c1cnn(C)c1] | H | F |
| 1472 | 4472 | 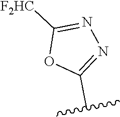 [SMILES: *c1nnc(C(F)F)o1] | CH₃ | 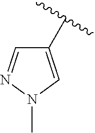 [SMILES: *c1cnn(C)c1] | H | F |
| 1473 | 4473 | 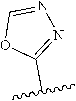 [SMILES: *c1nnco1] | CH₃ | 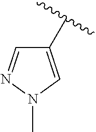 [SMILES: *c1cnn(C)c1] | H | F |
| 1474 | 4474 | 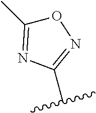 [SMILES: *c1noc(C)n1] | CH₃ | 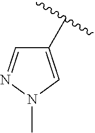 [SMILES: *c1cnn(C)c1] | H | F |
| 1475 | 4475 | 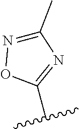 [SMILES: *c1nc(C)no1] | H | 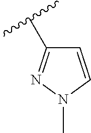 [SMILES: *c1ccn(C)n1] | H | F |
| 1476 | 4476 | 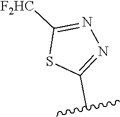 [SMILES: *c1nnc(C(F)F)s1] | H | 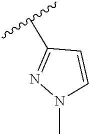 [SMILES: *c1ccn(C)n1] | H | F |
| 1477 | 4477 | 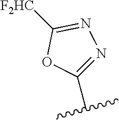 [SMILES: *c1nnc(C(F)F)o1] | H | 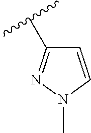 [SMILES: *c1ccn(C)n1] | H | F |
| 1478 | 4478 | 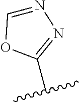 [SMILES: *c1nnco1] | H | 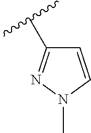 [SMILES: *c1ccn(C)n1] | H | F |
| 1479 | 4479 | 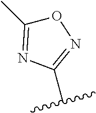 [SMILES: *c1noc(C)n1] | H | 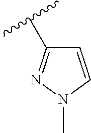 [SMILES: *c1ccn(C)n1] | H | F |
| 1480 | 4480 | 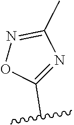 [SMILES: *c1nc(C)no1] | CH₃ | 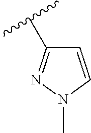 [SMILES: *c1ccn(C)n1] | H | F |
| 1481 | 4481 | 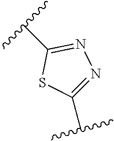 [SMILES: *c1nnc(*)s1] | CH₃ | 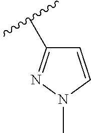 [SMILES: *c1ccn(C)n1] | H | F |
| 1482 | 4482 | 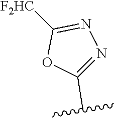 [SMILES: *c1nnc(C(F)F)o1] | CH₃ | 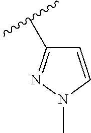 [SMILES: *c1ccn(C)n1] | H | F |
| 1483 | 4483 | 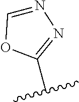 [SMILES: *c1nnco1] | CH₃ | 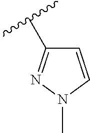 [SMILES: *c1ccn(C)n1] | H | F |
| 1484 | 4484 | 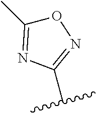 [SMILES: *c1noc(C)n1] | CH₃ | 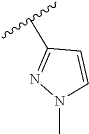 [SMILES: *c1ccn(C)n1] | H | F |
| 1485 | 4485 | 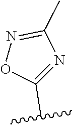 [SMILES: *c1nc(C)no1] | H | 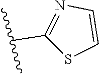 [SMILES: *c1nccs1] | H | F |
| 1486 | 4486 | 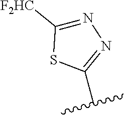 [SMILES: *c1nnc(C(F)F)s1] | H | 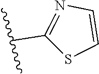 [SMILES: *c1nccs1] | H | F |
| 1487 | 4487 | 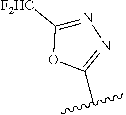 [SMILES: *c1nnc(C(F)F)o1] | H | 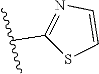 [SMILES: *c1nccs1] | H | F |
| 1488 | 4488 | 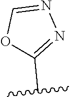 [SMILES: *c1nnco1] | H | 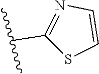 [SMILES: *c1nccs1] | H | F |
| 1489 | 4489 | 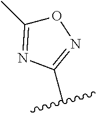 [SMILES: *c1noc(C)n1] | H | 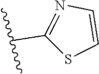 [SMILES: *c1nccs1] | H | F |
| 1490 | 4490 | 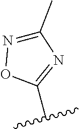 [SMILES: *c1nc(C)no1] | CH₃ | 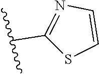 [SMILES: *c1nccs1] | H | F |
| 1491 | 4491 | 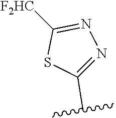 [SMILES: *c1nnc(C(F)F)s1] | CH₃ | 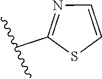 [SMILES: *c1nccs1] | H | F |
| 1492 | 4492 | 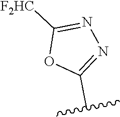 [SMILES: *c1nnc(C(F)F)o1] | CH₃ | 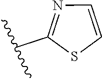 [SMILES: *c1nccs1] | H | F |
| 1493 | 4493 | 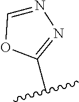 [SMILES: *c1nnco1] | CH₃ | 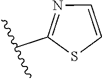 [SMILES: *c1nccs1] | H | F |
| 1494 | 4494 | 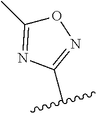 [SMILES: *c1noc(C)n1] | CH₃ | 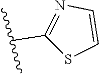 [SMILES: *c1nccs1] | H | F |
| 1495 | 4495 | 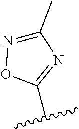 [SMILES: *c1nc(C)no1] | H | 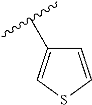 [SMILES: *c1ccsc1] | H | F |
| 1496 | 4496 | 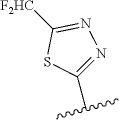 [SMILES: *c1nnc(C(F)F)s1] | H | 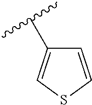 [SMILES: *c1ccsc1] | H | F |
| 1497 | 4497 | 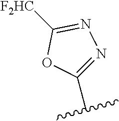 [SMILES: *c1nnc(C(F)F)o1] | H | 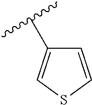 [SMILES: *c1ccsc1] | H | F |
| 1498 | 4498 | 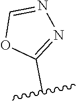 [SMILES: *c1nnco1] | H | 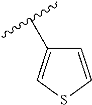 [SMILES: *c1ccsc1] | H | F |
| 1499 | 4499 | 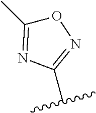 [SMILES: *c1noc(C)n1] | H | 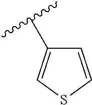 [SMILES: *c1ccsc1] | H | F |
| 1500 | 4500 | 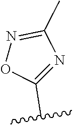 [SMILES: *c1nc(C)no1] | CH₃ | 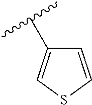 [SMILES: *c1ccsc1] | H | F |
| 1501 | 4501 | 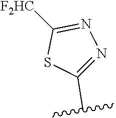 [SMILES: *c1nnc(C(F)F)s1] | CH₃ | 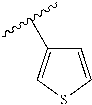 [SMILES: *c1ccsc1] | H | F |
| 1502 | 4502 | 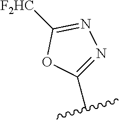 [SMILES: *c1nnc(C(F)F)o1] | CH₃ | 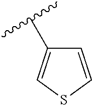 [SMILES: *c1ccsc1] | H | F |
| 1503 | 4503 | 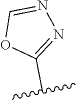 [SMILES: *c1nnco1] | CH₃ | 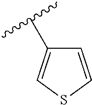 [SMILES: *c1ccsc1] | H | F |
| 1504 | 4504 | 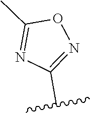 [SMILES: *c1noc(C)n1] | CH₃ | 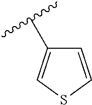 [SMILES: *c1ccsc1] | H | F |
| 1505 | 4505 | 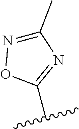 [SMILES: *c1nc(C)no1] | H | 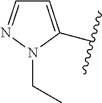 [SMILES: *c1ccnn1CC] | F | H |
| 1506 | 4506 | 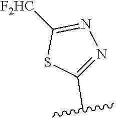 [SMILES: *c1nnc(C(F)F)s1] | H | 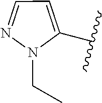 [SMILES: *c1ccnn1CC] | F | H |
| 1507 | 4507 | 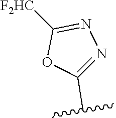 [SMILES: *c1nnc(C(F)F)o1] | H | 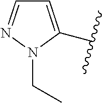 [SMILES: *c1ccnn1CC] | F | H |
| 1508 | 4508 | 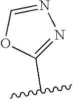 [SMILES: *c1nnco1] | H | 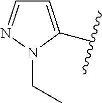 [SMILES: *c1ccnn1CC] | F | H |
| 1509 | 4509 | 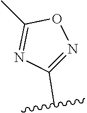 [SMILES: *c1noc(C)n1] | H | 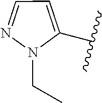 [SMILES: *c1ccnn1CC] | F | H |
| 1510 | 4510 | 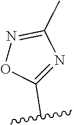 [SMILES: *c1nc(C)no1] | CH₃ | 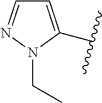 [SMILES: *c1ccnn1CC] | F | H |
| 1511 | 4511 | 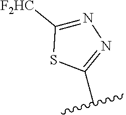 [SMILES: *c1nnc(C(F)F)s1] | CH₃ | 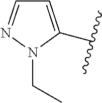 [SMILES: *c1ccnn1CC] | F | H |
| 1512 | 4512 | 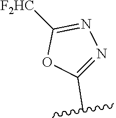 [SMILES: *c1nnc(C(F)F)o1] | CH₃ | 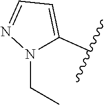 [SMILES: *c1ccnn1CC] | F | H |
| 1513 | 4513 | 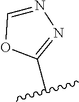 [SMILES: *c1nnco1] | CH₃ | 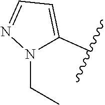 [SMILES: *c1ccnn1CC] | F | H |
| 1514 | 4514 | 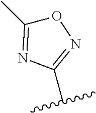 [SMILES: *c1noc(C)n1] | CH₃ | 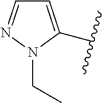 [SMILES: *c1ccnn1CC] | F | H |
| 1515 | 4515 | 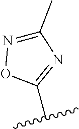 [SMILES: *c1nc(C)no1] | H | 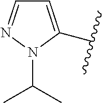 [SMILES: *c1ccnn1C(C)C] | F | H |
| 1516 | 4516 | 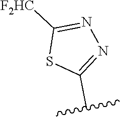 [SMILES: *c1nnc(C(F)F)s1] | H | 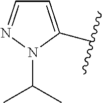 [SMILES: *c1ccnn1C(C)C] | F | H |
| 1517 | 4517 | 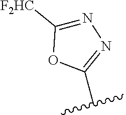 [SMILES: *c1nnc(C(F)F)o1] | H | 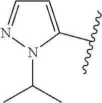 [SMILES: *c1ccnn1C(C)C] | F | H |
| 1518 | 4518 | 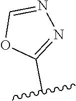 [SMILES: *c1nnco1] | H | 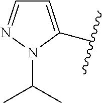 [SMILES: *c1ccnn1C(C)C] | F | H |
| 1519 | 4519 | 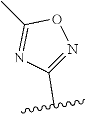 [SMILES: *c1noc(C)n1] | H | 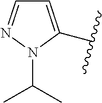 [SMILES: *c1ccnn1C(C)C] | F | H |
| 1520 | 4520 | 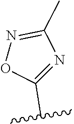 [SMILES: *c1nc(C)no1] | CH₃ | 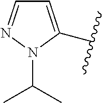 [SMILES: *c1ccnn1C(C)C] | F | H |
| 1521 | 4521 | 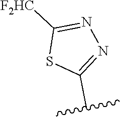 [SMILES: *c1nnc(C(F)F)s1] | CH₃ | 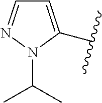 [SMILES: *c1ccnn1C(C)C] | F | H |
| 1522 | 4522 | 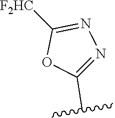 [SMILES: *c1nnc(C(F)F)o1] | CH₃ | 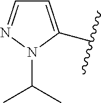 [SMILES: *c1ccnn1C(C)C] | F | H |
| 1523 | 4523 | 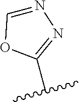 [SMILES: *c1nnco1] | CH₃ | 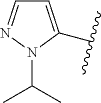 [SMILES: *c1ccnn1C(C)C] | F | H |
| 1524 | 4524 | 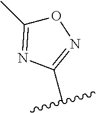 [SMILES: *c1noc(C)n1] | CH₃ | 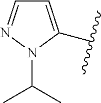 [SMILES: *c1ccnn1C(C)C] | F | H |
| 1525 | 4525 | 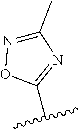 [SMILES: *c1nc(C)no1] | H | 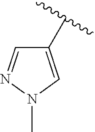 [SMILES: *c1cnn(C)c1] | F | H |
| 1526 | 4526 | 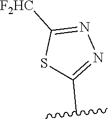 [SMILES: *c1nnc(C(F)F)s1] | H | 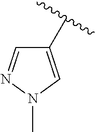 [SMILES: *c1cnn(C)c1] | F | H |
| 1527 | 4527 | 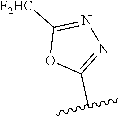 [SMILES: *c1nnc(C(F)F)o1] | H | 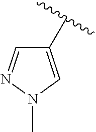 [SMILES: *c1cnn(C)c1] | F | H |
| 1528 | 4528 | 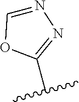 [SMILES: *c1nnco1] | H | 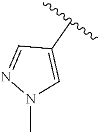 [SMILES: *c1cnn(C)c1] | F | H |
| 1529 | 4529 | 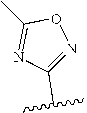 [SMILES: *c1noc(C)n1] | H | 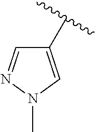 [SMILES: *c1cnn(C)c1] | F | H |
| 1530 | 4530 | 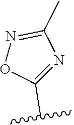 [SMILES: *c1nc(C)no1] | CH₃ | 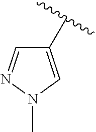 [SMILES: *c1cnn(C)c1] | F | H |
| 1531 | 4531 | 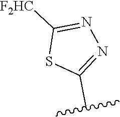 [SMILES: *c1nnc(C(F)F)s1] | CH₃ | 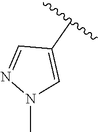 [SMILES: *c1cnn(C)c1] | F | H |
| 1532 | 4532 | 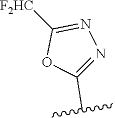 [SMILES: *c1nnc(C(F)F)o1] | CH₃ | 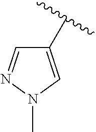 [SMILES: *c1cnn(C)c1] | F | H |
| 1533 | 4533 | 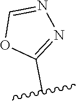 [SMILES: *c1nnco1] | CH₃ | 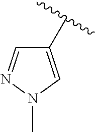 [SMILES: *c1cnn(C)c1] | F | H |
| 1534 | 4534 | 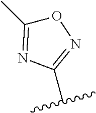 [SMILES: *c1noc(C)n1] | CH₃ | 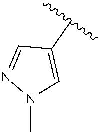 [SMILES: *c1cnn(C)c1] | F | H |
| 1535 | 4535 | 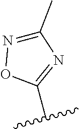 [SMILES: *c1nc(C)no1] | H | 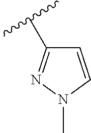 [SMILES: *c1ccn(C)n1] | F | H |
| 1536 | 4536 | 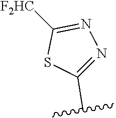 [SMILES: *c1nnc(C(F)F)s1] | H | 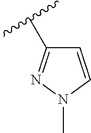 [SMILES: *c1ccn(C)n1] | F | H |
| 1537 | 4537 | 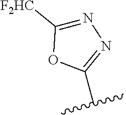 [SMILES: *c1nnc(C(F)F)o1] | H | 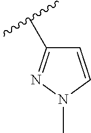 [SMILES: *c1ccn(C)n1] | F | H |
| 1538 | 4538 | 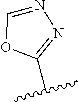 [SMILES: *c1nnco1] | H | 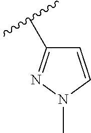 [SMILES: *c1ccn(C)n1] | F | H |
| 1539 | 4539 | 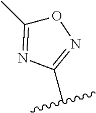 [SMILES: *c1noc(C)n1] | H | 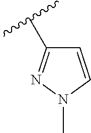 [SMILES: *c1ccn(C)n1] | F | H |
| 1540 | 4540 | 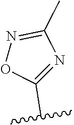 [SMILES: *c1nc(C)no1] | CH₃ | 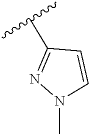 [SMILES: *c1ccn(C)n1] | F | H |
| 1541 | 4541 | 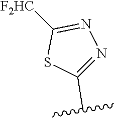 [SMILES: *c1nnc(C(F)F)s1] | CH₃ | 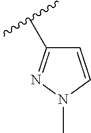 [SMILES: *c1ccn(C)n1] | F | H |
| 1542 | 4542 | 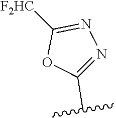 [SMILES: *c1nnc(C(F)F)o1] | CH₃ | 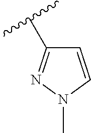 [SMILES: *c1ccn(C)n1] | F | H |
| 1543 | 4543 | 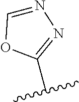 [SMILES: *c1nnco1] | CH₃ | 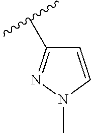 [SMILES: *c1ccn(C)n1] | F | H |
| 1544 | 4544 | 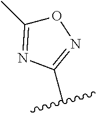 [SMILES: *c1noc(C)n1] | CH₃ | 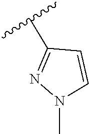 [SMILES: *c1ccn(C)n1] | F | H |
| 1545 | 4545 | 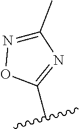 [SMILES: *c1nc(C)no1] | H | 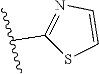 [SMILES: *c1nccs1] | F | H |
| 1546 | 4546 | 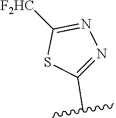 [SMILES: *c1nnc(C(F)F)s1] | H | 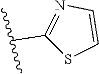 [SMILES: *c1nccs1] | F | H |
| 1547 | 4547 | 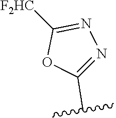 [SMILES: *c1nnc(C(F)F)o1] | H | 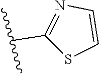 [SMILES: *c1nccs1] | F | H |
| 1548 | 4548 | 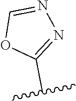 [SMILES: *c1nnco1] | H | 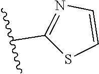 [SMILES: *c1nccs1] | F | H |
| 1549 | 4549 | 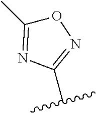 [SMILES: *c1noc(C)n1] | H | 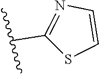 [SMILES: *c1nccs1] | F | H |
| 1550 | 4550 | 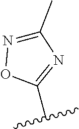 [SMILES: *c1nc(C)no1] | CH₃ | 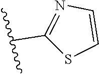 [SMILES: *c1nccs1] | F | H |
| 1551 | 4551 | 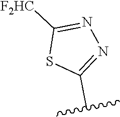 [SMILES: *c1nnc(C(F)F)s1] | CH₃ | 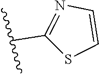 [SMILES: *c1nccs1] | F | H |
| 1552 | 4552 | 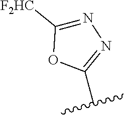 [SMILES: *c1nnc(C(F)F)o1] | CH₃ | 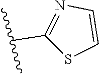 [SMILES: *c1nccs1] | F | H |
| 1553 | 4553 | 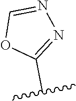 [SMILES: *c1nnco1] | CH₃ | 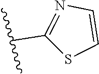 [SMILES: *c1nccs1] | F | H |
| 1554 | 4554 | 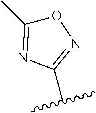 [SMILES: *c1noc(C)n1] | CH₃ | 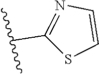 [SMILES: *c1nccs1] | F | H |
| 1555 | 4555 | 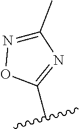 [SMILES: *c1nc(C)no1] | H | 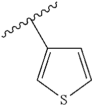 [SMILES: *c1ccsc1] | F | H |
| 1556 | 4556 | 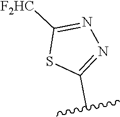 [SMILES: *c1nnc(C(F)F)s1] | H | 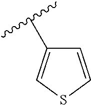 [SMILES: *c1ccsc1] | F | H |
| 1557 | 4557 | 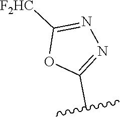 [SMILES: *c1nnc(C(F)F)o1] | H | 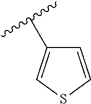 [SMILES: *c1ccsc1] | F | H |
| 1558 | 4558 | 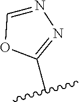 [SMILES: *c1nnco1] | H | 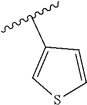 [SMILES: *c1ccsc1] | F | H |
| 1559 | 4559 | 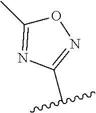 [SMILES: *c1noc(C)n1] | H | 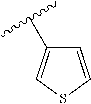 [SMILES: *c1ccsc1] | F | H |
| 1560 | 4560 | 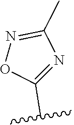 [SMILES: *c1nc(C)no1] | CH₃ | 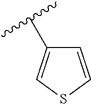 [SMILES: *c1ccsc1] | F | H |
| 1561 | 4561 | 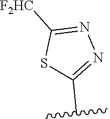 [SMILES: *c1nnc(C(F)F)s1] | CH₃ | 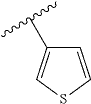 [SMILES: *c1ccsc1] | F | H |
| 1562 | 4562 | 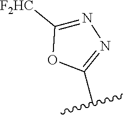 [SMILES: *c1nnc(C(F)F)o1] | CH₃ | 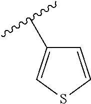 [SMILES: *c1ccsc1] | F | H |
| 1563 | 4563 | 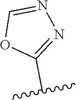 [SMILES: *c1nnco1] | CH₃ | 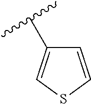 [SMILES: *c1ccsc1] | F | H |
| 1564 | 4564 | 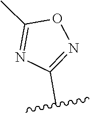 [SMILES: *c1noc(C)n1] | CH₃ | 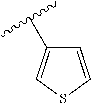 [SMILES: *c1ccsc1] | F | H |
| 1565 | 4565 | 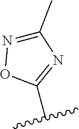 [SMILES: *c1nc(C)no1] | H | 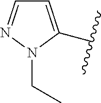 [SMILES: *c1ccnn1CC] | H | Cl |
| 1566 | 4566 | 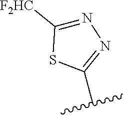 [SMILES: *c1nnc(C(F)F)s1] | H | 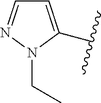 [SMILES: *c1ccnn1CC] | H | Cl |
| 1567 | 4567 | 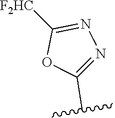 [SMILES: *c1nnc(C(F)F)o1] | H | 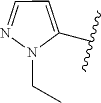 [SMILES: *c1ccnn1CC] | H | Cl |
| 1568 | 4568 | 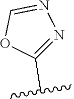 [SMILES: *c1nnco1] | H | 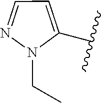 [SMILES: *c1ccnn1CC] | H | Cl |
| 1569 | 4569 | 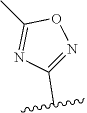 [SMILES: *c1noc(C)n1] | H | 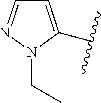 [SMILES: *c1ccnn1CC] | H | Cl |
| 1570 | 4570 | 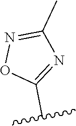 [SMILES: *c1nc(C)no1] | CH₃ | 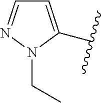 [SMILES: *c1ccnn1CC] | H | Cl |
| 1571 | 4571 | 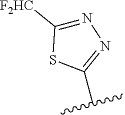 [SMILES: *c1nnc(C(F)F)s1] | CH₃ | 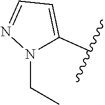 [SMILES: *c1ccnn1CC] | H | Cl |
| 1572 | 4572 | 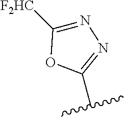 [SMILES: *c1nnc(C(F)F)o1] | CH₃ | 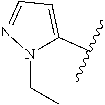 [SMILES: *c1ccnn1CC] | H | Cl |
| 1573 | 4573 | 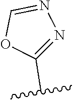 [SMILES: *c1nnco1] | CH₃ | 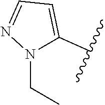 [SMILES: *c1ccnn1CC] | H | Cl |
| 1574 | 4574 | 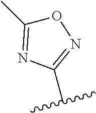 [SMILES: *c1noc(C)n1] | CH₃ | 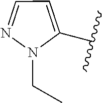 [SMILES: *c1ccnn1CC] | H | Cl |
| 1575 | 4575 | 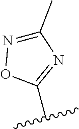 [SMILES: *c1nc(C)no1] | H | 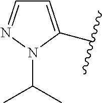 [SMILES: *c1ccnn1C(C)C] | H | Cl |
| 1576 | 4576 | 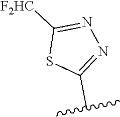 [SMILES: *c1nnc(C(F)F)s1] | H | 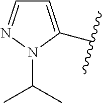 [SMILES: *c1ccnn1C(C)C] | H | Cl |
| 1577 | 4577 | 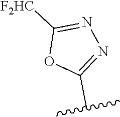 [SMILES: *c1nnc(C(F)F)o1] | H | 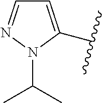 [SMILES: *c1ccnn1C(C)C] | H | Cl |
| 1578 | 4578 | 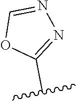 [SMILES: *c1nnco1] | H | 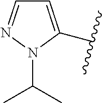 [SMILES: *c1ccnn1C(C)C] | H | Cl |
| 1579 | 4579 | 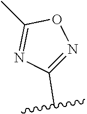 [SMILES: *c1noc(C)n1] | H | 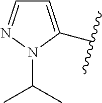 [SMILES: *c1ccnn1C(C)C] | H | Cl |
| 1580 | 4580 | 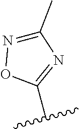 [SMILES: *c1nc(C)no1] | CH₃ | 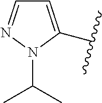 [SMILES: *c1ccnn1C(C)C] | H | Cl |
| 1581 | 4581 | 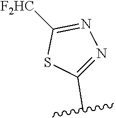 [SMILES: *c1nnc(C(F)F)s1] | CH₃ | 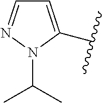 [SMILES: *c1ccnn1C(C)C] | H | Cl |
| 1582 | 4582 | 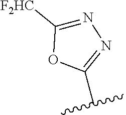 [SMILES: *c1nnc(C(F)F)o1] | CH₃ | 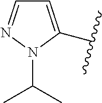 [SMILES: *c1ccnn1C(C)C] | H | Cl |
| 1583 | 4583 | 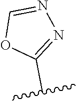 [SMILES: *c1nnco1] | CH₃ | 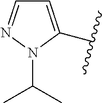 [SMILES: *c1ccnn1C(C)C] | H | Cl |
| 1584 | 4584 | 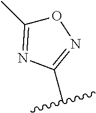 [SMILES: *c1noc(C)n1] | CH₃ | 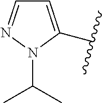 [SMILES: *c1ccnn1C(C)C] | H | Cl |
| 1585 | 4585 | 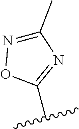 [SMILES: *c1nc(C)no1] | H | 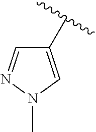 [SMILES: *c1cnn(C)c1] | H | Cl |
| 1586 | 4586 | 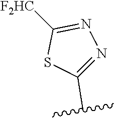 [SMILES: *c1nnc(C(F)F)s1] | H | 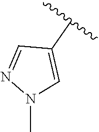 [SMILES: *c1cnn(C)c1] | H | Cl |
| 1587 | 4587 | 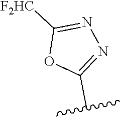 [SMILES: *c1nnc(C(F)F)o1] | H | 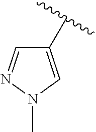 [SMILES: *c1cnn(C)c1] | H | Cl |
| 1588 | 4588 | 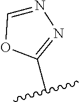 [SMILES: *c1nnco1] | H | 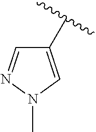 [SMILES: *c1cnn(C)c1] | H | Cl |
| 1589 | 4589 | 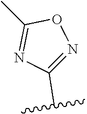 [SMILES: *c1noc(C)n1] | H | 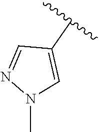 [SMILES: *c1cnn(C)c1] | H | Cl |
| 1590 | 4590 | 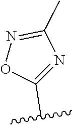 [SMILES: *c1nc(C)no1] | CH₃ | 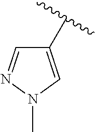 [SMILES: *c1cnn(C)c1] | H | Cl |
| 1591 | 4591 | 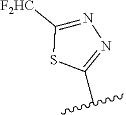 [SMILES: *c1nnc(C(F)F)s1] | CH₃ | 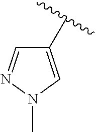 [SMILES: *c1cnn(C)c1] | H | Cl |
| 1592 | 4592 | 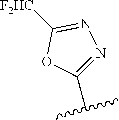 [SMILES: *c1nnc(C(F)F)o1] | CH₃ | 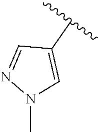 [SMILES: *c1cnn(C)c1] | H | Cl |
| 1593 | 4593 | 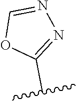 [SMILES: *c1nnco1] | CH₃ | 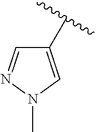 [SMILES: *c1cnn(C)c1] | H | Cl |
| 1594 | 4594 | 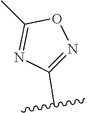 [SMILES: *c1noc(C)n1] | CH₃ | 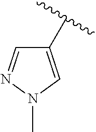 [SMILES: *c1cnn(C)c1] | H | Cl |
| 1595 | 4595 | 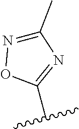 [SMILES: *c1nc(C)no1] | H | 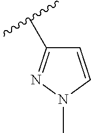 [SMILES: *c1ccn(C)n1] | H | Cl |
| 1596 | 4596 | 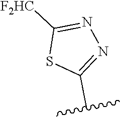 [SMILES: *c1nnc(C(F)F)s1] | H | 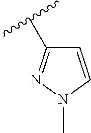 [SMILES: *c1ccn(C)n1] | H | Cl |
| 1597 | 4597 | 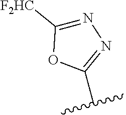 [SMILES: *c1nnc(C(F)F)o1] | H | 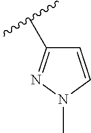 [SMILES: *c1ccn(C)n1] | H | Cl |
| 1598 | 4598 | 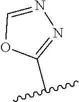 [SMILES: *c1nnco1] | H | 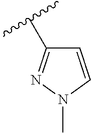 [SMILES: *c1ccn(C)n1] | H | CI |
| 1599 | 4599 | 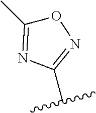 [SMILES: *c1noc(C)n1] | H | 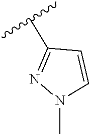 [SMILES: *c1ccn(C)n1] | H | Cl |
| 1600 | 4600 | 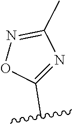 [SMILES: *c1nc(C)no1] | CH₃ | 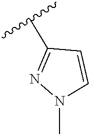 [SMILES: *c1ccn(C)n1] | H | Cl |
| 1601 | 4601 | 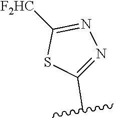 [SMILES: *c1nnc(C(F)F)s1] | CH₃ | 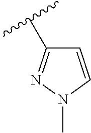 [SMILES: *c1ccn(C)n1] | H | Cl |
| 1602 | 4602 | 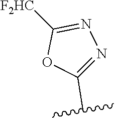 [SMILES: *c1nnc(C(F)F)o1] | CH₃ | 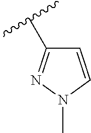 [SMILES: *c1ccn(C)n1] | H | Cl |
| 1603 | 4603 | 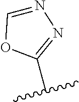 [SMILES: *c1nnco1] | CH₃ | 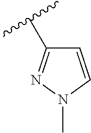 [SMILES: *c1ccn(C)n1] | H | Cl |
| 1604 | 4604 | 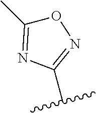 [SMILES: *c1noc(C)n1] | CH₃ | 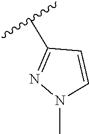 [SMILES: *c1ccn(C)n1] | H | Cl |
| 1605 | 4605 | 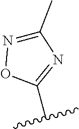 [SMILES: *c1nc(C)no1] | H | 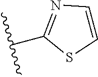 [SMILES: *c1nccs1] | H | Cl |
| 1606 | 4606 | 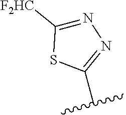 [SMILES: *c1nnc(C(F)F)s1] | H | 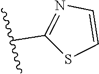 [SMILES: *c1nccs1] | H | Cl |
| 1607 | 4607 | 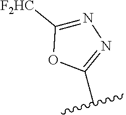 [SMILES: *c1nnc(C(F)F)o1] | H | 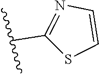 [SMILES: *c1nccs1] | H | Cl |
| 1608 | 4608 | 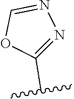 [SMILES: *c1nnco1] | H | 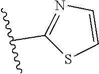 [SMILES: *c1nccs1] | H | Cl |
| 1609 | 4609 | 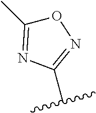 [SMILES: *c1noc(C)n1] | H | 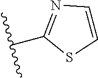 [SMILES: *c1nccs1] | H | Cl |
| 1610 | 4610 | 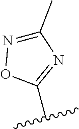 [SMILES: *c1nc(C)no1] | CH₃ | 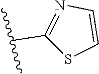 [SMILES: *c1nccs1] | H | Cl |
| 1611 | 4611 | 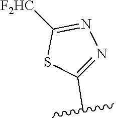 [SMILES: *c1nnc(C(F)F)s1] | CH₃ | 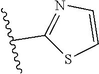 [SMILES: *c1nccs1] | H | Cl |
| 1612 | 4612 | 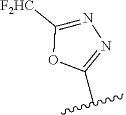 [SMILES: *c1nnc(C(F)F)o1] | CH₃ | 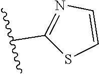 [SMILES: *c1nccs1] | H | Cl |
| 1613 | 4613 | 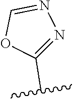 [SMILES: *c1nnco1] | CH₃ | 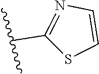 [SMILES: *c1nccs1] | H | Cl |
| 1614 | 4614 | 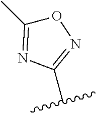 [SMILES: *c1noc(C)n1] | CH₃ | 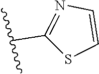 [SMILES: *c1nccs1] | H | Cl |
| 1615 | 4615 | 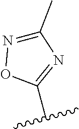 [SMILES: *c1nc(C)no1] | H | 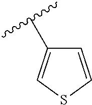 [SMILES: *c1ccsc1] | H | Cl |
| 1616 | 4616 | 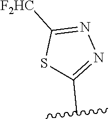 [SMILES: *c1nnc(C(F)F)s1] | H | 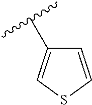 [SMILES: *c1ccsc1] | H | Cl |
| 1617 | 4617 | 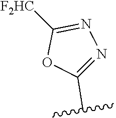 [SMILES: *c1nnc(C(F)F)o1] | H | 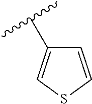 [SMILES: *c1ccsc1] | H | CI |
| 1618 | 4618 | 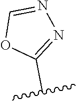 [SMILES: *c1nnco1] | H | 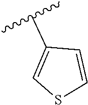 [SMILES: *c1ccsc1] | H | Cl |
| 1619 | 4619 | 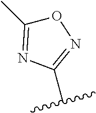 [SMILES: *c1noc(C)n1] | H | 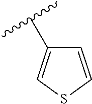 [SMILES: *c1ccsc1] | H | Cl |
| 1620 | 4620 | 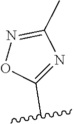 [SMILES: *c1nc(C)no1] | CH₃ | 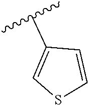 [SMILES: *c1ccsc1] | H | Cl |
| 1621 | 4621 | 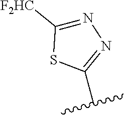 [SMILES: *c1nnc(C(F)F)s1] | CH₃ | 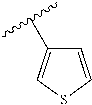 [SMILES: *c1ccsc1] | H | Cl |
| 1622 | 4622 | 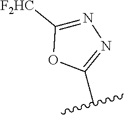 [SMILES: *c1nnc(C(F)F)o1] | CH₃ | 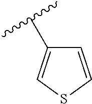 [SMILES: *c1ccsc1] | H | Cl |
| 1623 | 4623 | 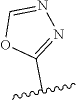 [SMILES: *c1nnco1] | CH₃ | 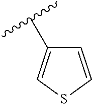 [SMILES: *c1ccsc1] | H | Cl |
| 1624 | 4624 | 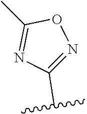 [SMILES: *c1noc(C)n1] | CH₃ | 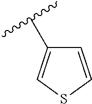 [SMILES: *c1ccsc1] | H | Cl |
| 1625 | 4625 | 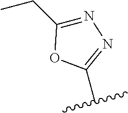 [SMILES: *c1nnc(CC)o1] | CH₃ | 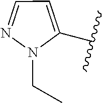 [SMILES: *c1ccnn1CC] | H | H |
| 1626 | 4626 | 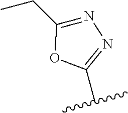 [SMILES: *c1nnc(CC)o1] | CH₃ | 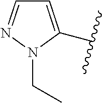 [SMILES: *c1ccnn1CC] | H | F |
| 1627 | 4627 | 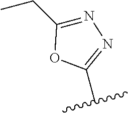 [SMILES: *c1nnc(CC)o1] | CH₃ | 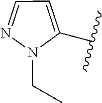 [SMILES: *c1ccnn1CC] | F | H |
| 1628 | 4628 | 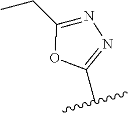 [SMILES: *c1nnc(CC)o1] | CH₃ | 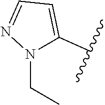 [SMILES: *c1ccnn1CC] | H | Cl |
| 1629 | 4629 | 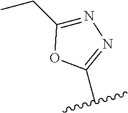 [SMILES: *c1nnc(CC)o1] | H | 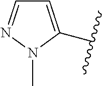 [SMILES: *c1ccnn1C] | H | H |
| 1630 | 4630 | 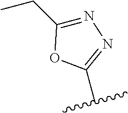 [SMILES: *c1nnc(CC)o1] | H | 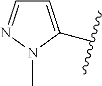 [SMILES: *c1ccnn1C] | H | F |
| 1631 | 4631 | 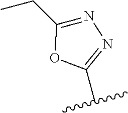 [SMILES: *c1nnc(CC)o1] | H | 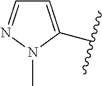 [SMILES: *c1ccnn1C] | F | H |
| 1632 | 4632 | 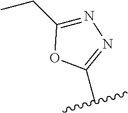 [SMILES: *c1nnc(CC)o1] | H | 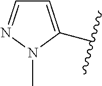 [SMILES: *c1ccnn1C] | H | Cl |
| 1633 | 4633 | 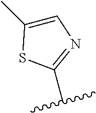 [SMILES: *c1ncc(C)s1] | H | 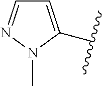 [SMILES: *c1ccnn1C] | H | H |
| 1634 | 4634 | 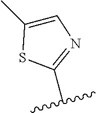 [SMILES: *c1ncc(C)s1] | CH₃ | 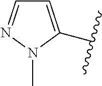 [SMILES: *c1ccnn1C] | H | H |
| 1635 | 4635 | 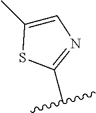 [SMILES: *c1ncc(C)s1] | H | 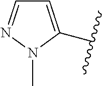 [SMILES: *c1ccnn1C] | H | F |
| 1636 | 4636 | 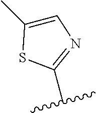 [SMILES: *c1ncc(C)s1] | CH₃ | 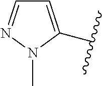 [SMILES: *c1ccnn1C] | H | F |
| 1637 | 4637 | 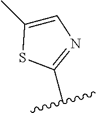 [SMILES: *c1ncc(C)s1] | H | 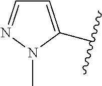 [SMILES: *c1ccnn1C] | F | H |
| 1638 | 4638 | 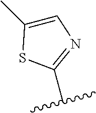 [SMILES: *c1ncc(C)s1] | CH₃ | 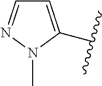 [SMILES: *c1ccnn1C] | F | H |
| 1639 | 4639 | 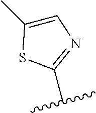 [SMILES: *c1ncc(C)s1] | H | 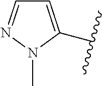 [SMILES: *c1ccnn1C] | H | Cl |
| 1640 | 4640 | 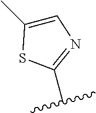 [SMILES: *c1ncc(C)s1] | CH₃ | 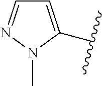 [SMILES: *c1ccnn1C] | H | Cl |
| 1641 | 4641 | 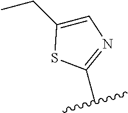 [SMILES: *c1ncc(CC)s1] | H | 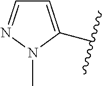 [SMILES: *c1ccnn1C] | H | H |
| 1642 | 4642 | 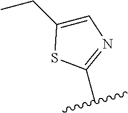 [SMILES: *c1ncc(CC)s1] | CH₃ | 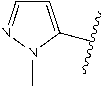 [SMILES: *c1ccnn1C] | H | H |
| 1643 | 4643 | 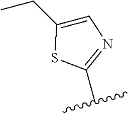 [SMILES: *c1ncc(CC)s1] | H | 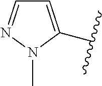 [SMILES: *c1ccnn1C] | H | F |
| 1644 | 4644 | 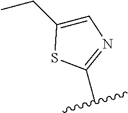 [SMILES: *c1ncc(CC)s1] | CH₃ | 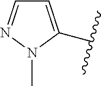 [SMILES: *c1ccnn1C] | H | F |
| 1645 | 4645 | 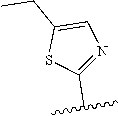 [SMILES: *c1ncc(CC)s1] | H | 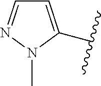 [SMILES: *c1ccnn1C] | F | H |
| 1646 | 4646 | 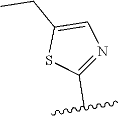 [SMILES: *c1ncc(CC)s1] | CH₃ | 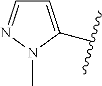 [SMILES: *c1ccnn1C] | F | H |
| 1647 | 4647 | 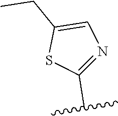 [SMILES: *c1ncc(CC)s1] | H | 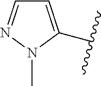 [SMILES: *c1ccnn1C] | H | Cl |
| 1648 | 4648 | 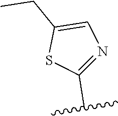 [SMILES: *c1ncc(CC)s1] | CH₃ | 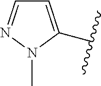 [SMILES: *c1ccnn1C] | H | Cl |
| 1649 | 4649 | 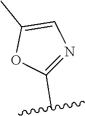 [SMILES: *c1ncc(C)o1] | H | 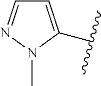 [SMILES: *c1ccnn1C] | H | H |
| 1650 | 4650 | 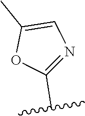 [SMILES: *c1ncc(C)o1] | CH₃ | 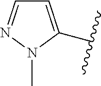 [SMILES: *c1ccnn1C] | H | H |
| 1651 | 4651 | 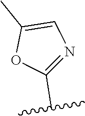 [SMILES: *c1ncc(C)o1] | H | 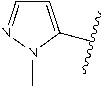 [SMILES: *c1ccnn1C] | H | F |
| 1652 | 4652 | 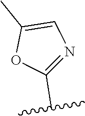 [SMILES: *c1ncc(C)o1] | CH₃ | 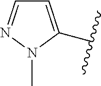 [SMILES: *c1ccnn1C] | H | F |
| 1653 | 4653 | 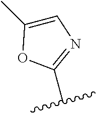 [SMILES: *c1ncc(C)o1] | H | 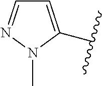 [SMILES: *c1ccnn1C] | F | H |
| 1654 | 4654 | 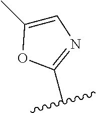 [SMILES: *c1ncc(C)o1] | CH₃ | 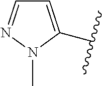 [SMILES: *c1ccnn1C] | F | H |
| 1655 | 4655 | 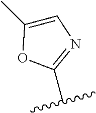 [SMILES: *c1ncc(C)o1] | H | 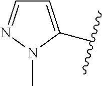 [SMILES: *c1ccnn1C] | H | Cl |
| 1656 | 4656 | 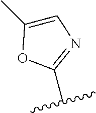 [SMILES: *c1ncc(C)o1] | CH₃ | 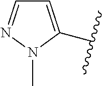 [SMILES: *c1ccnn1C] | H | Cl |
| 1657 | 4657 | 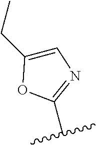 [SMILES: *c1ncc(CC)o1] | H | 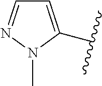 [SMILES: *c1ccnn1C] | H | H |
| 1658 | 4658 | 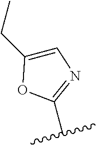 [SMILES: *c1ncc(CC)o1] | CH₃ | 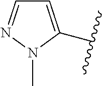 [SMILES: *c1ccnn1C] | H | H |
| 1659 | 4659 | 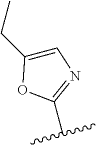 [SMILES: *c1ncc(CC)o1] | H | 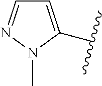 [SMILES: *c1ccnn1C] | H | F |
| 1660 | 4660 | 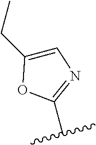 [SMILES: *c1ncc(CC)o1] | CH₃ | 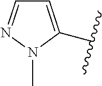 [SMILES: *c1ccnn1C] | H | F |
| 1661 | 4661 | 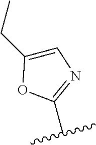 [SMILES: *c1ncc(CC)o1] | H | 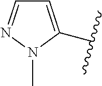 [SMILES: *c1ccnn1C] | F | H |
| 1662 | 4662 | 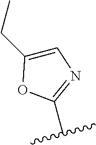 [SMILES: *c1ncc(CC)o1] | CH₃ | 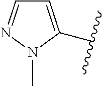 [SMILES: *c1ccnn1C] | F | H |
| 1663 | 4663 | 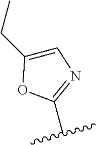 [SMILES: *c1ncc(CC)o1] | H | 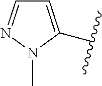 [SMILES: *c1ccnn1C] | H | Cl |
| 1664 | 4664 | 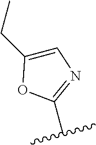 [SMILES: *c1ncc(CC)o1] | CH₃ | 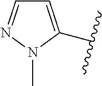 [SMILES: *c1ccnn1C] | H | Cl |
| 1665 | 4665 | 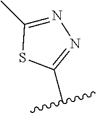 [SMILES: *c1nnc(C)s1] | H | 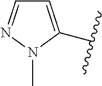 [SMILES: *c1ccnn1C] | H | H |
| 1666 | 4666 | 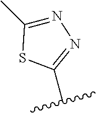 [SMILES: *c1nnc(C)s1] | CH₃ | 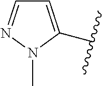 [SMILES: *c1ccnn1C] | H | H |
| 1667 | 4667 | 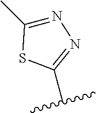 [SMILES: *c1nnc(C)s1] | H | 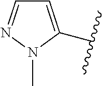 [SMILES: *c1ccnn1C] | H | F |
| 1668 | 4668 | 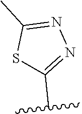 [SMILES: *c1nnc(C)s1] | CH₃ | 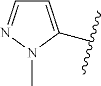 [SMILES: *c1ccnn1C] | H | F |
| 1669 | 4669 | 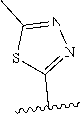 [SMILES: *c1nnc(C)s1] | H | 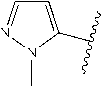 [SMILES: *c1ccnn1C] | F | H |
| 1670 | 4670 | 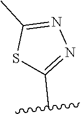 [SMILES: *c1nnc(C)s1] | CH₃ | 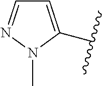 [SMILES: *c1ccnn1C] | F | H |
| 1671 | 4671 | 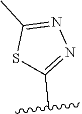 [SMILES: *c1nnc(C)s1] | H | 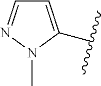 [SMILES: *c1ccnn1C] | H | Cl |
| 1672 | 4672 | 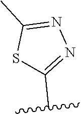 [SMILES: *c1nnc(C)s1] | CH₃ | 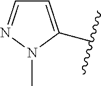 [SMILES: *c1ccnn1C] | H | Cl |
| 1673 | 4673 | 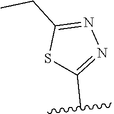 [SMILES: *c1nnc(CC)s1] | H | 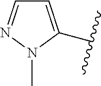 [SMILES: *c1ccnn1C] | H | H |
| 1674 | 4674 | 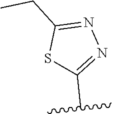 [SMILES: *c1nnc(CC)s1] | CH₃ | 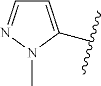 [SMILES: *c1ccnn1C] | H | H |
| 1675 | 4675 | 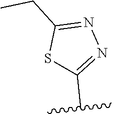 [SMILES: *c1nnc(CC)s1] | H | 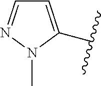 [SMILES: *c1ccnn1C] | H | F |
| 1676 | 4676 | 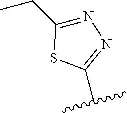 [SMILES: *c1nnc(CC)s1] | CH₃ | 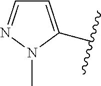 [SMILES: *c1ccnn1C] | H | F |
| 1677 | 4677 | 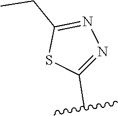 [SMILES: *c1nnc(CC)s1] | H | 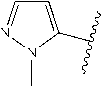 [SMILES: *c1ccnn1C] | F | H |
| 1678 | 4678 | 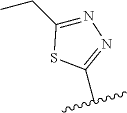 [SMILES: *c1nnc(CC)s1] | CH₃ | 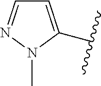 [SMILES: *c1ccnn1C] | F | H |
| 1679 | 4679 | 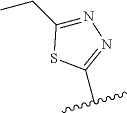 [SMILES: *c1nnc(CC)s1] | H | 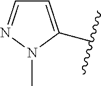 [SMILES: *c1ccnn1C] | H | Cl |
| 1680 | 4680 | 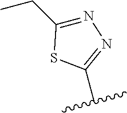 [SMILES: *c1nnc(CC)s1] | CH₃ | 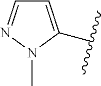 [SMILES: *c1ccnn1C] | H | Cl |
| 1681 | 4681 | 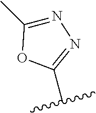 [SMILES: *c1nnc(C)o1] | H | 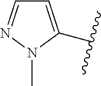 [SMILES: *c1ccnn1C] | H | H |
| 1682 | 4682 | 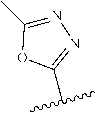 [SMILES: *c1nnc(C)o1] | CH₃ | 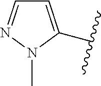 [SMILES: *c1ccnn1C] | H | H |
| 1683 | 4683 | 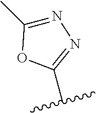 [SMILES: *c1nnc(C)o1] | H | 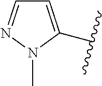 [SMILES: *c1ccnn1C] | H | F |
| 1684 | 4684 | 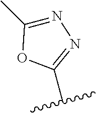 [SMILES: *c1nnc(C)o1] | CH₃ | 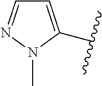 [SMILES: *c1ccnn1C] | H | F |
| 1685 | 4685 | 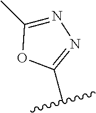 [SMILES: *c1nnc(C)o1] | H | 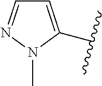 [SMILES: *c1ccnn1C] | F | H |
| 1686 | 4686 | 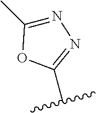 [SMILES: *c1nnc(C)o1] | CH₃ | 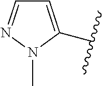 [SMILES: *c1ccnn1C] | F | H |
| 1687 | 4687 | 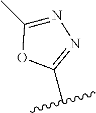 [SMILES: *c1nnc(C)o1] | H | 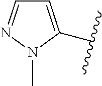 [SMILES: *c1ccnn1C] | H | Cl |
| 1689 | 4688 | 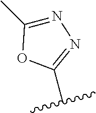 [SMILES: *c1nnc(C)o1] | CH₃ | 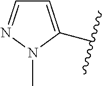 [SMILES: *c1ccnn1C] | H | Cl |
| 1690 | 4689 | 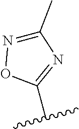 [SMILES: *c1nc(C)no1] | H | 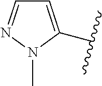 [SMILES: *c1ccnn1C] | H | H |
| 1691 | 4690 | 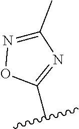 [SMILES: *c1nc(C)no1] | CH₃ | 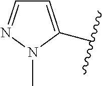 [SMILES: *c1ccnn1C] | H | H |
| 1692 | 4691 | 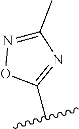 [SMILES: *c1nc(C)no1] | H | 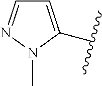 [SMILES: *c1ccnn1C] | H | F |
| 1693 | 4692 | 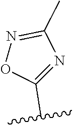 [SMILES: *c1nc(C)no1] | CH₃ | 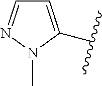 [SMILES: *c1ccnn1C] | H | F |
| 1694 | 4693 | 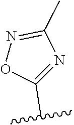 [SMILES: *c1nc(C)no1] | H | 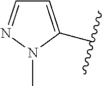 [SMILES: *c1ccnn1C] | F | H |
| 1695 | 4694 | 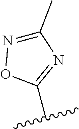 [SMILES: *c1nc(C)no1] | CH₃ | 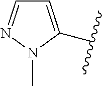 [SMILES: *c1ccnn1C] | F | H |
| 1696 | 4695 | 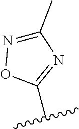 [SMILES: *c1nc(C)no1] | H | 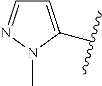 [SMILES: *c1ccnn1C] | H | Cl |
| 1697 | 4696 | 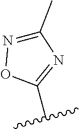 [SMILES: *c1nc(C)no1] | CH₃ | 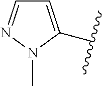 [SMILES: *c1ccnn1C] | H | Cl |
| 1698 | 4697 | 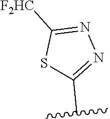 [SMILES: *c1nnc(C(F)F)s1] | H | 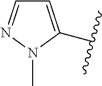 [SMILES: *c1ccnn1C] | H | H |
| 1699 | 4698 | 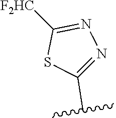 [SMILES: *c1nnc(C(F)F)s1] | CH₃ | 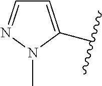 [SMILES: *c1ccnn1C] | H | H |
| 1700 | 4699 | 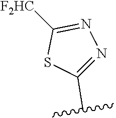 [SMILES: *c1nnc(C(F)F)s1] | H | 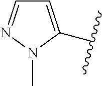 [SMILES: *c1ccnn1C] | H | F |
| 1701 | 4700 | 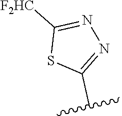 [SMILES: *c1nnc(C(F)F)s1] | CH₃ | 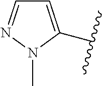 [SMILES: *c1ccnn1C] | H | F |
| 1702 | 4701 | 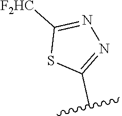 [SMILES: *c1nnc(C(F)F)s1] | H | 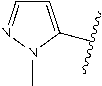 [SMILES: *c1ccnn1C] | F | H |
| 1703 | 4702 | 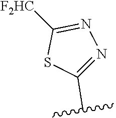 [SMILES: *c1nnc(C(F)F)s1] | CH₃ | 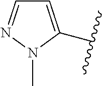 [SMILES: *c1ccnn1C] | F | H |
| 1704 | 4703 | 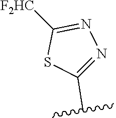 [SMILES: *c1nnc(C(F)F)s1] | H | 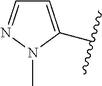 [SMILES: *c1ccnn1C] | H | Cl |
| 1705 | 4704 | 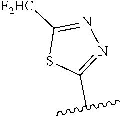 [SMILES: *c1nnc(C(F)F)s1] | CH₃ | 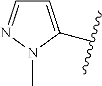 [SMILES: *c1ccnn1C] | H | Cl |
| 1706 | 4705 | 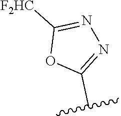 [SMILES: *c1nnc(C(F)F)o1] | H | 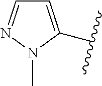 [SMILES: *c1ccnn1C] | H | H |
| 1707 | 4706 | 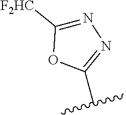 [SMILES: *c1nnc(C(F)F)o1] | CH₃ | 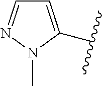 [SMILES: *c1ccnn1C] | H | H |
| 1708 | 4707 | 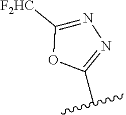 [SMILES: *c1nnc(C(F)F)o1] | H | 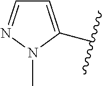 [SMILES: *c1ccnn1C] | H | F |
| 1709 | 4708 | 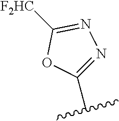 [SMILES: *c1nnc(C(F)F)o1] | CH₃ | 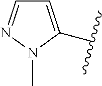 [SMILES: *c1ccnn1C] | H | F |
| 1710 | 4709 | 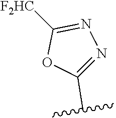 [SMILES: *c1nnc(C(F)F)o1] | H | 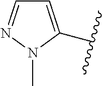 [SMILES: *c1ccnn1C] | F | H |
| 1711 | 4710 | 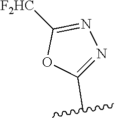 [SMILES: *c1nnc(C(F)F)o1] | CH₃ | 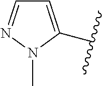 [SMILES: *c1ccnn1C] | F | H |
| 1712 | 4711 | 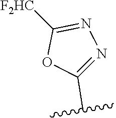 [SMILES: *c1nnc(C(F)F)o1] | H | 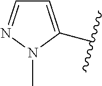 [SMILES: *c1ccnn1C] | H | Cl |
| 1713 | 4712 | 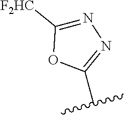 [SMILES: *c1nnc(C(F)F)o1] | CH₃ | 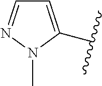 [SMILES: *c1ccnn1C] | H | Cl |
| 1714 | 4713 | 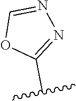 [SMILES: *c1nnco1] | H | 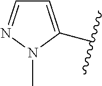 [SMILES: *c1ccnn1C] | H | H |
| 1715 | 4714 | 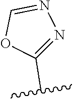 [SMILES: *c1nnco1] | CH₃ | 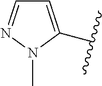 [SMILES: *c1ccnn1C] | H | H |
| 1716 | 4715 | 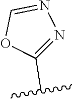 [SMILES: *c1nnco1] | H | 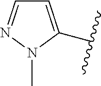 [SMILES: *c1ccnn1C] | H | F |
| 1717 | 4716 | 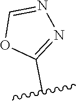 [SMILES: *c1nnco1] | CH₃ | 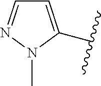 [SMILES: *c1ccnn1C] | H | F |
| 1718 | 4717 | 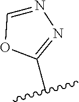 [SMILES: *c1nnco1] | H | 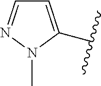 [SMILES: *c1ccnn1C] | F | H |
| 1719 | 4718 | 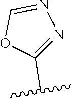 [SMILES: *c1nnco1] | CH₃ | 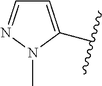 [SMILES: *c1ccnn1C] | F | H |
| 1720 | 4719 | 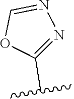 [SMILES: *c1nnco1] | H | 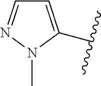 [SMILES: *c1ccnn1C] | H | Cl |
| 1721 | 4720 | 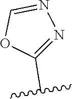 [SMILES: *c1nnco1] | CH₃ | 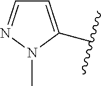 [SMILES: *c1ccnn1C] | H | Cl |
| 1722 | 4721 | 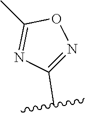 [SMILES: *c1noc(C)n1] | H | 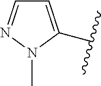 [SMILES: *c1ccnn1C] | H | H |
| 1723 | 4722 | 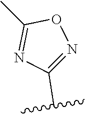 [SMILES: *c1noc(C)n1] | CH₃ | 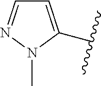 [SMILES: *c1ccnn1C] | H | H |
| 1724 | 4723 | 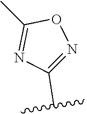 [SMILES: *c1noc(C)n1] | H | 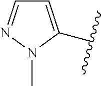 [SMILES: *c1ccnn1C] | H | F |
| 1725 | 4724 | 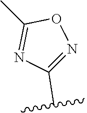 [SMILES: *c1noc(C)n1] | CH₃ | 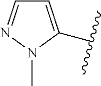 [SMILES: *c1ccnn1C] | H | F |
| 1726 | 4725 | 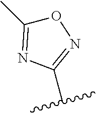 [SMILES: *c1noc(C)n1] | H | 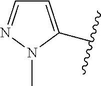 [SMILES: *c1ccnn1C] | F | H |
| 1727 | 4726 | 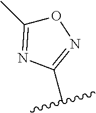 [SMILES: *c1noc(C)n1] | CH₃ | 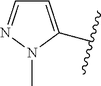 [SMILES: *c1ccnn1C] | F | H |
| 1728 | 4727 | 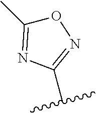 [SMILES: *c1noc(C)n1] | H | 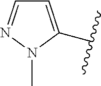 [SMILES: *c1ccnn1C] | H | Cl |
| 1729 | 4728 | 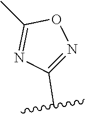 [SMILES: *c1noc(C)n1] | CH₃ | 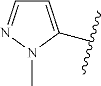 [SMILES: *c1ccnn1C] | H | Cl |

24. The compound of any one of claims 1-4, wherein the compound of formula (A), formula (I), or formula (II) is a compound having any one of formula 2001-2337, or a pharmaceutically acceptable salt, solvate, hydrate, cocrystal, or prodrug thereof:

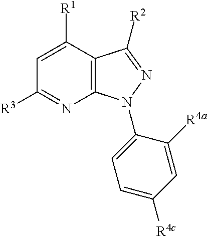

| Cpd. # | R¹ | R² | R³ | R^{4a} | R^{4c} |
|---|---|---|---|---|---|
| 2001 | 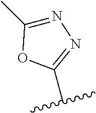 [SMILES: *c1nnc(C)o1] | CHF₂ | 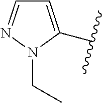 [SMILES: *c1ccnn1CC] | H | H |
| 2002 | 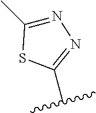 [SMILES: *c1nnc(C)s1] | CHF₂ | 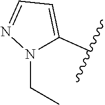 [SMILES: *c1ccnn1CC] | H | H |
| 2003 | 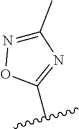 [SMILES: *c1nc(C)no1] | CHF₂ | 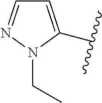 [SMILES: *c1ccnn1CC] | H | H |
| 2004 | 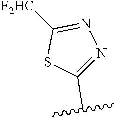 [SMILES: *c1nnc(C(F)F)s1] | CHF₂ | 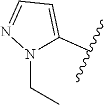 [SMILES: *c1ccnn1CC] | H | H |
| 2005 | 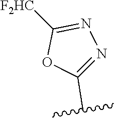 [SMILES: *c1nnc(C(F)F)o1] | CHF₂ | 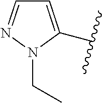 [SMILES: *c1ccnn1CC] | H | H |
| 2006 | 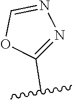 [SMILES: *c1nnco1] | CHF₂ | 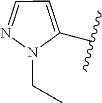 [SMILES: *c1ccnn1CC] | H | H |
| 2007 | 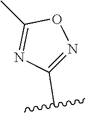 [SMILES: *c1noc(C)n1] | CHF₂ | 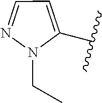 [SMILES: *c1ccnn1CC] | H | H |
| 2008 | 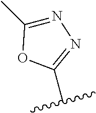 [SMILES: *c1nnc(C)o1] | CF₃ | 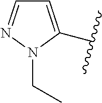 [SMILES: *c1ccnn1CC] | H | H |
| 2009 | 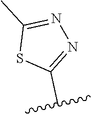 [SMILES: *c1nnc(C)s1] | CF₃ | 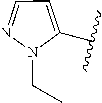 [SMILES: *c1ccnn1CC] | H | H |
| 2010 | 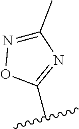 [SMILES: *c1nc(C)no1] | CF₃ | 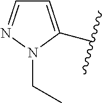 [SMILES: *c1ccnn1CC] | H | H |
| 2011 | 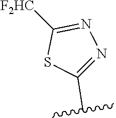 [SMILES: *c1nnc(C(F)F)s1] | CF₃ | 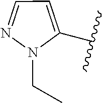 [SMILES: *c1ccnn1CC] | H | H |
| 2012 | 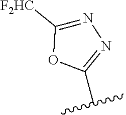 [SMILES: *c1nnc(C(F)F)o1] | CF₃ | 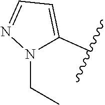 [SMILES: *c1ccnn1CC] | H | H |
| 2013 | 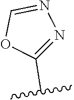 [SMILES: *c1nnco1] | CF₃ | 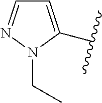 [SMILES: *c1ccnn1CC] | H | H |
| 2014 | 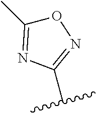 [SMILES: *c1noc(C)n1] | CF₃ | 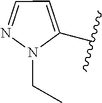 [SMILES: *c1ccnn1CC] | H | H |
| 2015 | 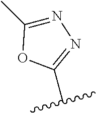 [SMILES: *c1nnc(C)o1] | CHF₂ | 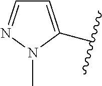 [SMILES: *c1ccnn1C] | H | H |
| 2016 | 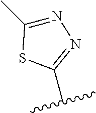 [SMILES: *c1nnc(C)s1] | CHF₂ | 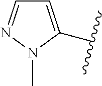 [SMILES: *c1ccnn1C] | H | H |
| 2017 | 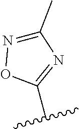 [SMILES: *c1nc(C)no1] | CHF₂ | 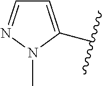 [SMILES: *c1ccnn1C] | H | H |
| 2018 | 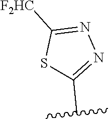 [SMILES: *c1nnc(C(F)F)s1] | CHF₂ | 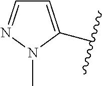 [SMILES: *c1ccnn1C] | H | H |
| 2019 | 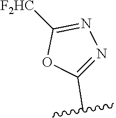 [SMILES: *c1nnc(C(F)F)o1] | CHF₂ | 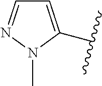 [SMILES: *c1ccnn1C] | H | H |
| 2020 | 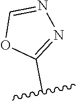 [SMILES: *c1nnco1] | CHF₂ | 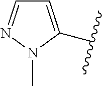 [SMILES: *c1ccnn1C] | H | H |
| 2021 | 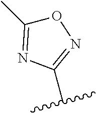 [SMILES: *c1noc(C)n1] | CHF₂ | 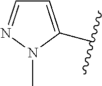 [SMILES: *c1ccnn1C] | H | H |
| 2022 | 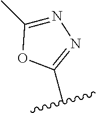 [SMILES: *c1nnc(C)o1] | CF₃ | 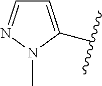 [SMILES: *c1ccnn1C] | H | H |
| 2023 | 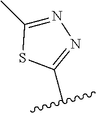 [SMILES: *c1nnc(C)s1] | CF₃ | 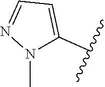 [SMILES: *c1ccnn1C] | H | H |
| 2024 | 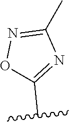 [SMILES: *c1nc(C)no1] | CF₃ | 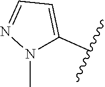 [SMILES: *c1ccnn1C] | H | H |
| 2025 | 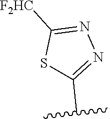 [SMILES: *c1nnc(C(F)F)s1] | CF₃ | 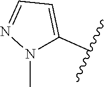 [SMILES: *c1ccnn1C] | H | H |
| 2026 | 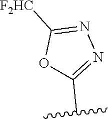 [SMILES: *c1nnc(C(F)F)o1] | CF₃ | 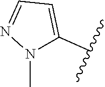 [SMILES: *c1ccnn1C] | H | H |
| 2027 | 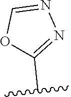 [SMILES: *c1nnco1] | CF₃ | 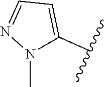 [SMILES: *c1ccnn1C] | H | H |
| 2028 | 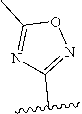 [SMILES: *c1noc(C)n1] | CF₃ | 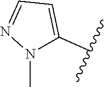 [SMILES: *c1ccnn1C] | H | H |
| 2029 | 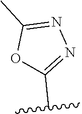 [SMILES: *c1nnc(C)o1] | CHF₂ | 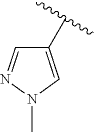 [SMILES: *c1cnn(C)c1] | H | H |
| 2030 | 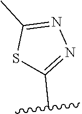 [SMILES: *c1nnc(C)s1] | CHF₂ | 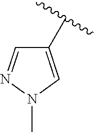 [SMILES: *c1cnn(C)c1] | H | H |
| 2031 | 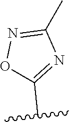 [SMILES: *c1nc(C)no1] | CHF₂ | 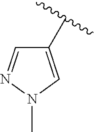 [SMILES: *c1cnn(C)c1] | H | H |
| 2032 | 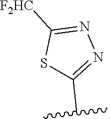 [SMILES: *c1nnc(C(F)F)s1] | CHF₂ | 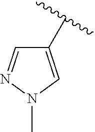 [SMILES: *c1cnn(C)c1] | H | H |
| 2033 | 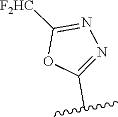 [SMILES: *c1nnc(C(F)F)o1] | CHF₂ | 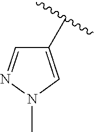 [SMILES: *c1cnn(C)c1] | H | H |
| 2034 | 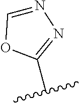 [SMILES: *c1nnco1] | CHF₂ | 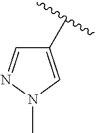 [SMILES: *c1cnn(C)c1] | H | H |
| 2035 | 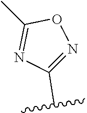 [SMILES: *c1noc(C)n1] | CHF₂ | 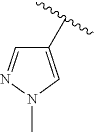 [SMILES: *c1cnn(C)c1] | H | H |
| 2036 | 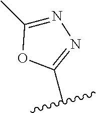 [SMILES: *c1nnc(C)o1] | CF₃ | 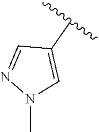 [SMILES: *c1cnn(C)c1] | H | H |
| 2037 | 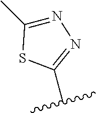 [SMILES: *c1nnc(C)s1] | CF₃ | 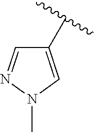 [SMILES: *c1cnn(C)c1] | H | H |
| 2038 | 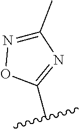 [SMILES: *c1nc(C)no1] | CF₃ | 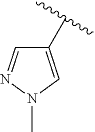 [SMILES: *c1cnn(C)c1] | H | H |
| 2039 | 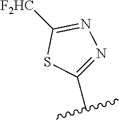 [SMILES: *c1nnc(C(F)F)s1] | CF₃ | 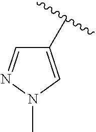 [SMILES: *c1cnn(C)c1] | H | H |
| 2040 | 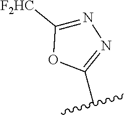 [SMILES: *c1nnc(C(F)F)o1] | CF₃ | 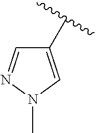 [SMILES: *c1cnn(C)c1] | H | H |
| 2041 | 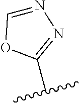 [SMILES: *c1nnco1] | CF₃ | 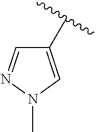 [SMILES: *c1cnn(C)c1] | H | H |
| 2042 | 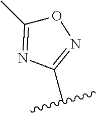 [SMILES: *c1noc(C)n1] | CF₃ | 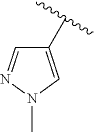 [SMILES: *c1cnn(C)c1] | H | H |
| 2043 | 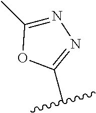 [SMILES: *c1nnc(C)o1] | CHF₂ | 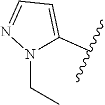 [SMILES: *c1ccnn1CC] | H | F |
| 2044 | 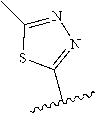 [SMILES: *c1nnc(C)s1] | CHF₂ | 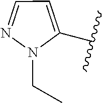 [SMILES: *c1ccnn1CC] | H | F |
| 2045 | 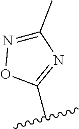 [SMILES: *c1nc(C)no1] | CHF₂ | 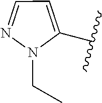 [SMILES: *c1ccnn1CC] | H | F |
| 2046 | 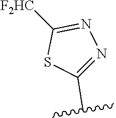 [SMILES: *c1nnc(C(F)F)s1] | CHF₂ | 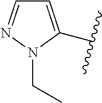 [SMILES: *c1ccnn1CC] | H | F |
| 2046 | 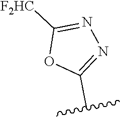 [SMILES: *c1nnc(C(F)F)o1] | CHF₂ | 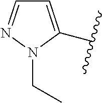 [SMILES: *c1ccnn1CC] | H | F |
| 2047 | 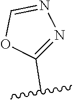 [SMILES: *c1nnco1] | CHF₂ | 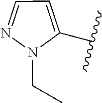 [SMILES: *c1ccnn1CC] | H | F |
| 2048 | 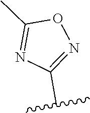 [SMILES: *c1noc(C)n1] | CHF₂ | 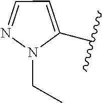 [SMILES: *c1ccnn1CC] | H | F |
| 2049 | 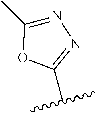 [SMILES: *c1nnc(C)o1] | CF₃ | 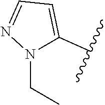 [SMILES: *c1ccnn1CC] | H | F |
| 2050 | 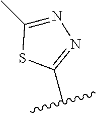 [SMILES: *c1nnc(C)s1] | CF₃ | 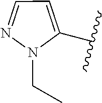 [SMILES: *c1ccnn1CC] | H | F |
| 2051 | 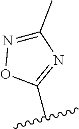 [SMILES: *c1nc(C)no1] | CF₃ | 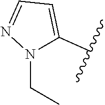 [SMILES: *c1ccnn1CC] | H | F |
| 2052 | 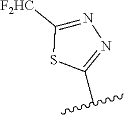 [SMILES: *c1nnc(C(F)F)s1] | CF₃ | 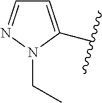 [SMILES: *c1ccnn1CC] | H | F |
| 2053 | 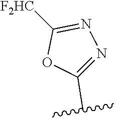 [SMILES: *c1nnc(C(F)F)o1] | CF₃ | 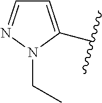 [SMILES: *c1ccnn1CC] | H | F |
| 2054 | 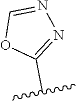 [SMILES: *c1nnco1] | CF₃ | 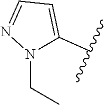 [SMILES: *c1ccnn1CC] | H | F |
| 2055 | 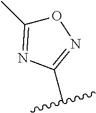 [SMILES: *c1noc(C)n1] | CF₃ | 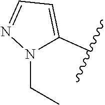 [SMILES: *c1ccnn1CC] | H | F |
| 2056 | 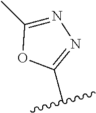 [SMILES: *c1nnc(C)o1] | CHF₂ | 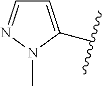 [SMILES: *c1ccnn1C] | H | F |
| 2057 | 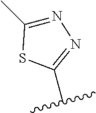 [SMILES: *c1nnc(C)s1] | CHF₂ | 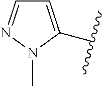 [SMILES: *c1ccnn1C] | H | F |
| 2058 | 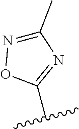 [SMILES: *c1nc(C)no1] | CHF₂ | 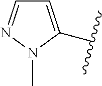 [SMILES: *c1ccnn1C] | H | F |
| 2059 | 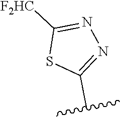 [SMILES: *c1nnc(C(F)F)s1] | CHF₂ | 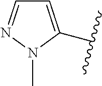 [SMILES: *c1ccnn1C] | H | F |
| 2060 | 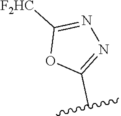 [SMILES: *c1nnc(C(F)F)o1] | CHF₂ | 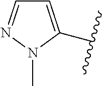 [SMILES: *c1ccnn1C] | H | F |
| 2061 | 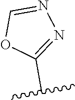 [SMILES: *c1nnco1] | CHF₂ | 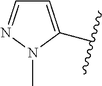 [SMILES: *c1ccnn1C] | H | F |
| 2062 | 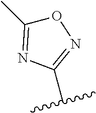 [SMILES: *c1noc(C)n1] | CHF₂ | 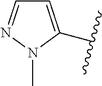 [SMILES: *c1ccnn1C] | H | F |
| 2063 | 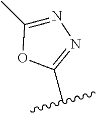 [SMILES: *c1nnc(C)o1] | CF₃ | 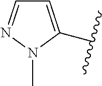 [SMILES: *c1ccnn1C] | H | F |
| 2064 | 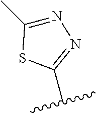 [SMILES: *c1nnc(C)s1] | CF₃ | 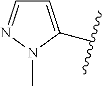 [SMILES: *c1ccnn1C] | H | F |
| 2065 | 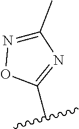 [SMILES: *c1nc(C)no1] | CF₃ | 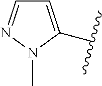 [SMILES: *c1ccnn1C] | H | F |
| 2066 | 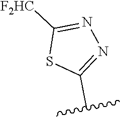 [SMILES: *c1nnc(C(F)F)s1] | CF₃ | 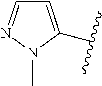 [SMILES: *c1ccnn1C] | H | F |
| 2067 | 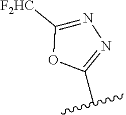 [SMILES: *c1nnc(C(F)F)o1] | CF₃ | 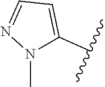 [SMILES: *c1ccnn1C] | H | F |
| 2068 | 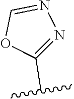 [SMILES: *c1nnco1] | CF₃ | 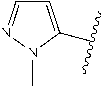 [SMILES: *c1ccnn1C] | H | F |
| 2069 | 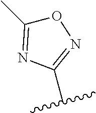 [SMILES: *c1noc(C)n1] | CF₃ | 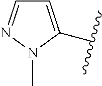 [SMILES: *c1ccnn1C] | H | F |
| 2070 | 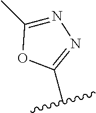 [SMILES: *c1nnc(C)o1] | CHF₂ | 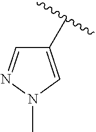 [SMILES: *c1cnn(C)c1] | H | F |
| 2071 | 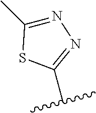 [SMILES: *c1nnc(C)s1] | CHF₂ | 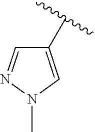 [SMILES: *c1cnn(C)c1] | H | F |
| 2072 | 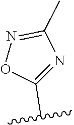 [SMILES: *c1nc(C)no1] | CHF₂ | 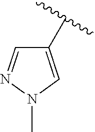 [SMILES: *c1cnn(C)c1] | H | F |
| 2073 | 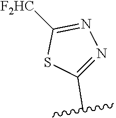 [SMILES: *c1nnc(C(F)F)s1] | CHF₂ | 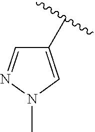 [SMILES: *c1cnn(C)c1] | H | F |
| 2074 | 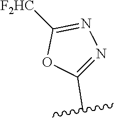 [SMILES: *c1nnc(C(F)F)o1] | CHF₂ | 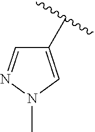 [SMILES: *c1cnn(C)c1] | H | F |
| 2075 | 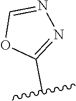 [SMILES: *c1nnco1] | CHF₂ | 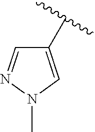 [SMILES: *c1cnn(C)c1] | H | F |
| 2076 | 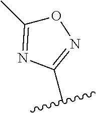 [SMILES: *c1noc(C)n1] | CHF₂ | 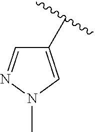 [SMILES: *c1cnn(C)c1] | H | F |
| 2077 | 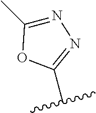 [SMILES: *c1nnc(C)o1] | CF₃ | 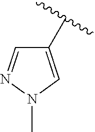 [SMILES: *c1cnn(C)c1] | H | F |
| 2078 | 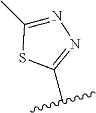 [SMILES: *c1nnc(C)s1] | CF₃ | 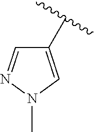 [SMILES: *c1cnn(C)c1] | H | F |
| 2079 | 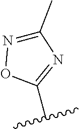 [SMILES: *c1nc(C)no1] | CF₃ | 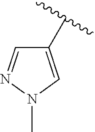 [SMILES: *c1cnn(C)c1] | H | F |
| 2080 | 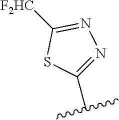 [SMILES: *c1nnc(C(F)F)s1] | CF₃ | 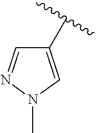 [SMILES: *c1cnn(C)c1] | H | F |
| 2081 | 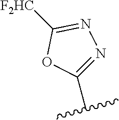 [SMILES: *c1nnc(C(F)F)o1] | CF₃ | 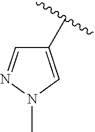 [SMILES: *c1cnn(C)c1] | H | F |
| 2082 | 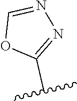 [SMILES: *c1nnco1] | CF₃ | 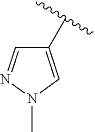 [SMILES: *c1cnn(C)c1] | H | F |
| 2083 | 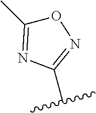 [SMILES: *c1noc(C)n1] | CF₃ | 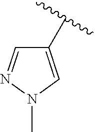 [SMILES: *c1cnn(C)c1] | H | F |
| 2084 | 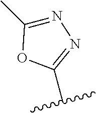 [SMILES: *c1nnc(C)o1] | CHF₂ | 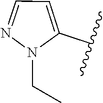 [SMILES: *c1ccnn1CC] | F | H |
| 2085 | 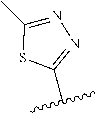 [SMILES: *c1nnc(C)s1] | CHF₂ | 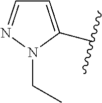 [SMILES: *c1ccnn1CC] | F | H |
| 2086 | 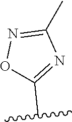 [SMILES: *c1nc(C)no1] | CHF₂ | 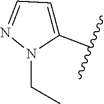 [SMILES: *c1ccnn1CC] | F | H |
| 2087 | 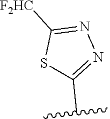 [SMILES: *c1nnc(C(F)F)s1] | CHF₂ | 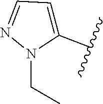 [SMILES: *c1ccnn1CC] | F | H |
| 2088 | 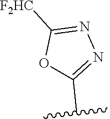 [SMILES: *c1nnc(C(F)F)o1] | CHF₂ | 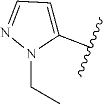 [SMILES: *c1ccnn1CC] | F | H |
| 2089 | 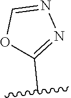 [SMILES: *c1nnco1] | CHF₂ | 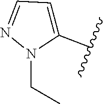 [SMILES: *c1ccnn1CC] | F | H |
| 2090 | 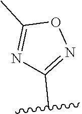 [SMILES: *c1noc(C)n1] | CHF₂ | 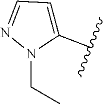 [SMILES: *c1ccnn1CC] | F | H |
| 2091 | 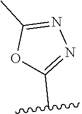 [SMILES: *c1nnc(C)o1] | CF₃ | 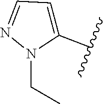 [SMILES: *c1ccnn1CC] | F | H |
| 2092 | 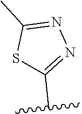 [SMILES: *c1nnc(C)s1] | CF₃ | 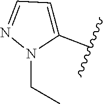 [SMILES: *c1ccnn1CC] | F | H |
| 2093 | 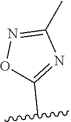 [SMILES: *c1nc(C)no1] | CF₃ | 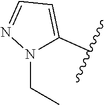 [SMILES: *c1ccnn1CC] | F | H |
| 2094 | 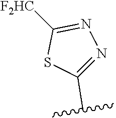 [SMILES: *c1nnc(C(F)F)s1] | CF₃ | 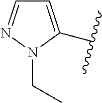 [SMILES: *c1ccnn1CC] | F | H |
| 2095 | 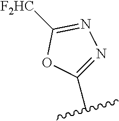 [SMILES: *c1nnc(C(F)F)o1] | CF₃ | 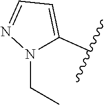 [SMILES: *c1ccnn1CC] | F | H |
| 2096 | 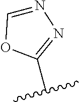 [SMILES: *c1nnco1] | CF₃ | 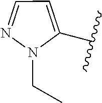 [SMILES: *c1ccnn1CC] | F | H |
| 2097 | 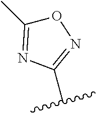 [SMILES: *c1noc(C)n1] | CF₃ | 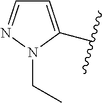 [SMILES: *c1ccnn1CC] | F | H |
| 2098 | 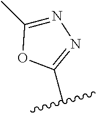 [SMILES: *c1nnc(C)o1] | CHF₂ | 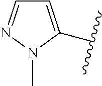 [SMILES: *c1ccnn1C] | F | H |
| 2099 | 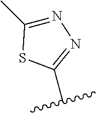 [SMILES: *c1nnc(C)s1] | CHF₂ | 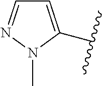 [SMILES: *c1ccnn1C] | F | H |
| 2100 | 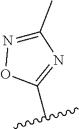 [SMILES: *c1nc(C)no1] | CHF₂ | 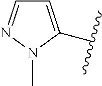 [SMILES: *c1ccnn1C] | F | H |
| 2101 | 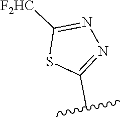 [SMILES: *c1nnc(C(F)F)s1] | CHF₂ | 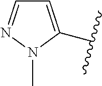 [SMILES: *c1ccnn1C] | F | H |
| 2102 | 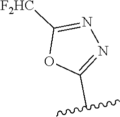 [SMILES: *c1nnc(C(F)F)o1] | CHF₂ | 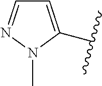 [SMILES: *c1ccnn1C] | F | H |
| 2103 | 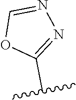 [SMILES: *c1nnco1] | CHF₂ | 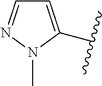 [SMILES: *c1ccnn1C] | F | H |
| 2104 | 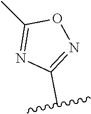 [SMILES: *c1noc(C)n1] | CHF₂ | 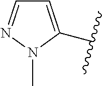 [SMILES: *c1ccnn1C] | F | H |
| 2105 | 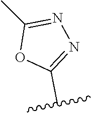 [SMILES: *c1nnc(C)o1] | CF₃ | 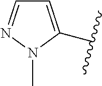 [SMILES: *c1ccnn1C] | F | H |
| 2106 | 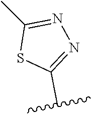 [SMILES: *c1nnc(C)s1] | CF₃ | 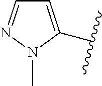 [SMILES: *c1ccnn1C] | F | H |
| 2107 | 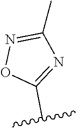 [SMILES: *c1nc(C)no1] | CF₃ | 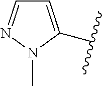 [SMILES: *c1ccnn1C] | F | H |
| 2108 | 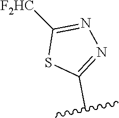 [SMILES: *c1nnc(C(F)F)s1] | CF₃ | 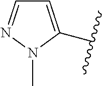 [SMILES: *c1ccnn1C] | F | H |
| 2109 | 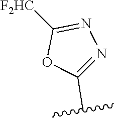 [SMILES: *c1nnc(C(F)F)o1] | CF₃ | 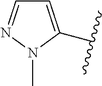 [SMILES: *c1ccnn1C] | F | H |
| 2110 | 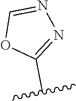 [SMILES: *c1nnco1] | CF₃ | 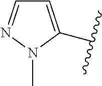 [SMILES: *c1ccnn1C] | F | H |
| 2111 | 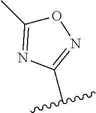 [SMILES: *c1noc(C)n1] | CF₃ | 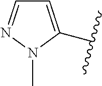 [SMILES: *c1ccnn1C] | F | H |
| 2112 | 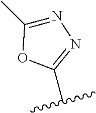 [SMILES: *c1nnc(C)o1] | CHF₂ | 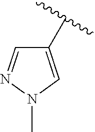 [SMILES: *c1cnn(C)c1] | F | H |
| 2113 | 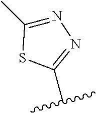 [SMILES: *c1nnc(C)s1] | CHF₂ | 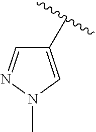 [SMILES: *c1cnn(C)c1] | F | H |
| 2114 | 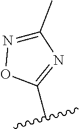 [SMILES: *c1nc(C)no1] | CHF₂ | 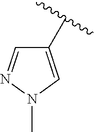 [SMILES: *c1cnn(C)c1] | F | H |
| 2115 | 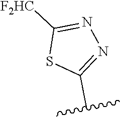 [SMILES: *c1nnc(C(F)F)s1] | CHF₂ | 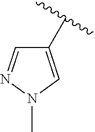 [SMILES: *c1cnn(C)c1] | F | H |
| 2116 | 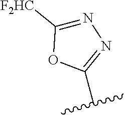 [SMILES: *c1nnc(C(F)F)o1] | CHF₂ | 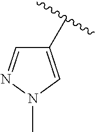 [SMILES: *c1cnn(C)c1] | F | H |
| 2117 | 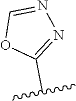 [SMILES: *c1nnco1] | CHF₂ | 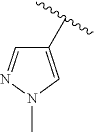 [SMILES: *c1cnn(C)c1] | F | H |
| 2118 | 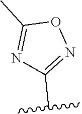 [SMILES: *c1noc(C)n1] | CHF₂ | 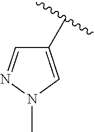 [SMILES: *c1cnn(C)c1] | F | H |
| 2119 | 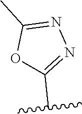 [SMILES: *c1nnc(C)o1] | CF₃ | 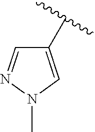 [SMILES: *c1cnn(C)c1] | F | H |
| 2120 | 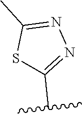 [SMILES: *c1nnc(C)s1] | CF₃ | 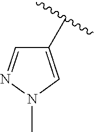 [SMILES: *c1cnn(C)c1] | F | H |
| 2121 | 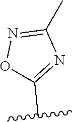 [SMILES: *c1nc(C)no1] | CF₃ | 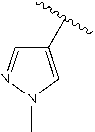 [SMILES: *c1cnn(C)c1] | F | H |
| 2122 | 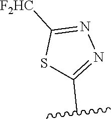 [SMILES: *c1nnc(C(F)F)s1] | CF₃ | 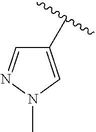 [SMILES: *c1cnn(C)c1] | F | H |
| 2123 | 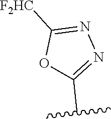 [SMILES: *c1nnc(C(F)F)o1] | CF₃ | 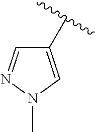 [SMILES: *c1cnn(C)c1] | F | H |
| 2124 | 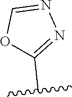 [SMILES: *c1nnco1] | CF₃ | 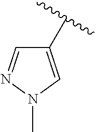 [SMILES: *c1cnn(C)c1] | F | H |
| 2125 | 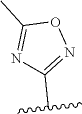 [SMILES: *c1noc(C)n1] | CF₃ | 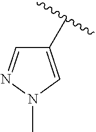 [SMILES: *c1cnn(C)c1] | F | H |
| 2126 | 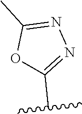 [SMILES: *c1nnc(C)o1] | CHF₂ | 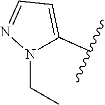 [SMILES: *c1ccnn1CC] | F | F |
| 2127 | 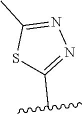 [SMILES: *c1nnc(C)s1] | CHF₂ | 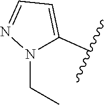 [SMILES: *c1ccnn1CC] | F | F |
| 2128 | 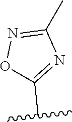 [SMILES: *c1nc(C)no1] | CHF₂ | 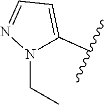 [SMILES: *c1ccnn1CC] | F | F |
| 2129 | 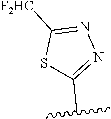 [SMILES: *c1nnc(C(F)F)s1] | CHF₂ | 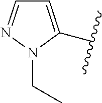 [SMILES: *c1ccnn1CC] | F | F |
| 2130 | 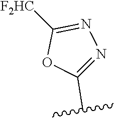 [SMILES: *c1nnc(C(F)F)o1] | CHF₂ | 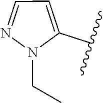 [SMILES: *c1ccnn1CC] | F | F |
| 2131 | 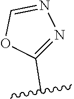 [SMILES: *c1nnco1] | CHF₂ | 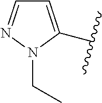 [SMILES: *c1ccnn1CC] | F | F |
| 2132 | 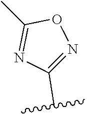 [SMILES: *c1noc(C)n1] | CHF₂ | 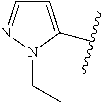 [SMILES: *c1ccnn1CC] | F | F |
| 2133 | 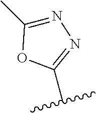 [SMILES: *c1nnc(C)o1] | CF₃ | 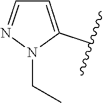 [SMILES: *c1ccnn1CC] | F | F |
| 2134 | 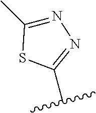 [SMILES: *c1nnc(C)s1] | CF₃ | 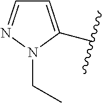 [SMILES: *c1ccnn1CC] | F | F |
| 2135 | 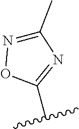 [SMILES: *c1nc(C)no1] | CF₃ | 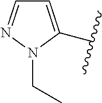 [SMILES: *c1ccnn1CC] | F | F |
| 2136 | 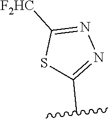 [SMILES: *c1nnc(C(F)F)s1] | CF₃ | 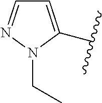 [SMILES: *c1ccnn1CC] | F | F |
| 2137 | 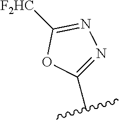 [SMILES: *c1nnc(C(F)F)o1] | CF₃ | 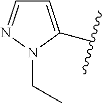 [SMILES: *c1ccnn1CC] | F | F |
| 2138 | 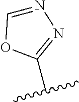 [SMILES: *c1nnco1] | CF₃ | 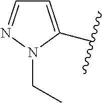 [SMILES: *c1ccnn1CC] | F | F |
| 2139 | 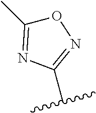 [SMILES: *c1noc(C)n1] | CF₃ | 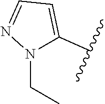 [SMILES: *c1ccnn1CC] | F | F |
| 2140 | 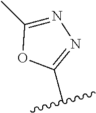 [SMILES: *c1nnc(C)o1] | CHF₂ | 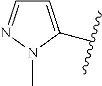 [SMILES: *c1ccnn1C] | F | F |
| 2141 | 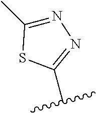 [SMILES: *c1nnc(C)s1] | CHF₂ | 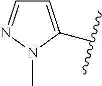 [SMILES: *c1ccnn1C] | F | F |
| 2142 | 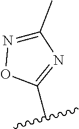 [SMILES: *c1nc(C)no1] | CHF₂ | 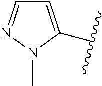 [SMILES: *c1ccnn1C] | F | F |
| 2143 | 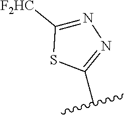 [SMILES: *c1nnc(C(F)F)s1] | CHF₂ | 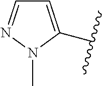 [SMILES: *c1ccnn1C] | F | F |
| 2144 | 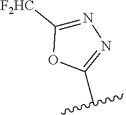 [SMILES: *c1nnc(C(F)F)o1] | CHF₂ | 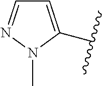 [SMILES: *c1ccnn1C] | F | F |
| 2145 | 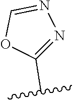 [SMILES: *c1nnco1] | CHF₂ | 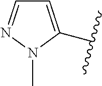 [SMILES: *c1ccnn1C] | F | F |
| 2146 | 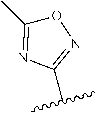 [SMILES: *c1noc(C)n1] | CHF₂ | 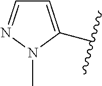 [SMILES: *c1ccnn1C] | F | F |
| 2147 | 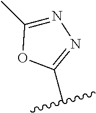 [SMILES: *c1nnc(C)o1] | CF₃ | 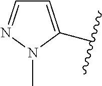 [SMILES: *c1ccnn1C] | F | F |
| 2148 | 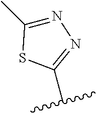 [SMILES: *c1nnc(C)s1] | CF₃ | 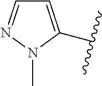 [SMILES: *c1ccnn1C] | F | F |
| 2149 | 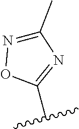 [SMILES: *c1nc(C)no1] | CF₃ | 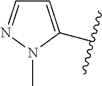 [SMILES: *c1ccnn1C] | F | F |
| 2150 | 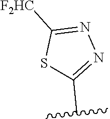 [SMILES: *c1nnc(C(F)F)s1] | CF₃ | 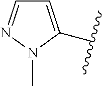 [SMILES: *c1ccnn1C] | F | F |
| 2151 | 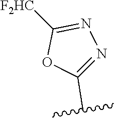 [SMILES: *c1nnc(C(F)F)o1] | CF₃ | 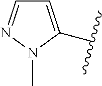 [SMILES: *c1ccnn1C] | F | F |
| 2152 | 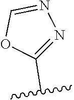 [SMILES: *c1nnco1] | CF₃ | 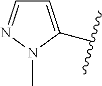 [SMILES: *c1ccnn1C] | F | F |
| 2153 | 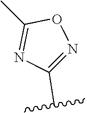 [SMILES: *c1noc(C)n1] | CF₃ | 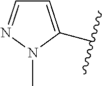 [SMILES: *c1ccnn1C] | F | F |
| 2154 | 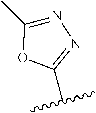 [SMILES: *c1nnc(C)o1] | CHF₂ | 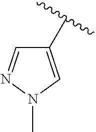 [SMILES: *c1cnn(C)c1] | F | F |
| 2155 | 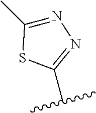 [SMILES: *c1nnc(C)s1] | CHF₂ | 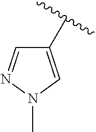 [SMILES: *c1cnn(C)c1] | F | F |
| 2156 | 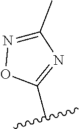 [SMILES: *c1nc(C)no1] | CHF₂ | 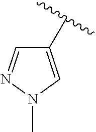 [SMILES: *c1cnn(C)c1] | F | F |
| 2157 | 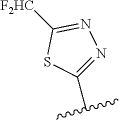 [SMILES: *c1nnc(C(F)F)s1] | CHF₂ | 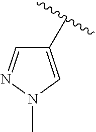 [SMILES: *c1cnn(C)c1] | F | F |
| 2158 | 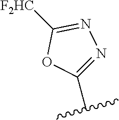 [SMILES: *c1nnc(C(F)F)o1] | CHF₂ | 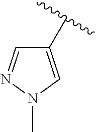 [SMILES: *c1cnn(C)c1] | F | F |
| 2159 | 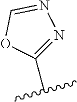 [SMILES: *c1nnco1] | CHF₂ | 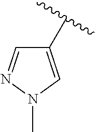 [SMILES: *c1cnn(C)c1] | F | F |
| 2160 | 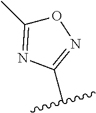 [SMILES: *c1noc(C)n1] | CHF₂ | 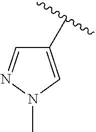 [SMILES: *c1cnn(C)c1] | F | F |
| 2161 | 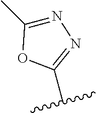 [SMILES: *c1nnc(C)o1] | CF₃ | 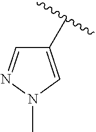 [SMILES: *c1cnn(C)c1] | F | F |
| 2162 | 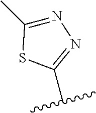 [SMILES: *c1nnc(C)s1] | CF₃ | 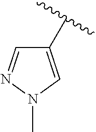 [SMILES: *c1cnn(C)c1] | F | F |
| 2163 | 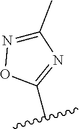 [SMILES: *c1nc(C)no1] | CF₃ | 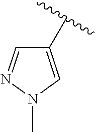 [SMILES: *c1cnn(C)c1] | F | F |
| 2164 | 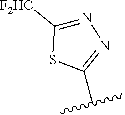 [SMILES: *c1nnc(C(F)F)s1] | CF₃ | 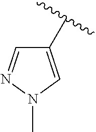 [SMILES: *c1cnn(C)c1] | F | F |
| 2165 | 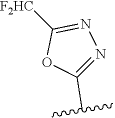 [SMILES: *c1nnc(C(F)F)o1] | CF₃ | 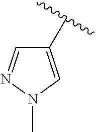 [SMILES: *c1cnn(C)c1] | F | F |
| 2166 | 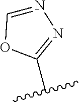 [SMILES: *c1nnco1] | CF₃ | 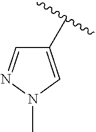 [SMILES: *c1cnn(C)c1] | F | F |
| 2167 | 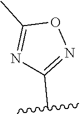 [SMILES: *c1noc(C)n1] | CF₃ | 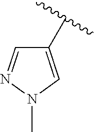 [SMILES: *c1cnn(C)c1] | F | F |
| 2168 | 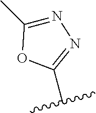 [SMILES: *c1nnc(C)o1] | CHF₂ | 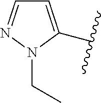 [SMILES: *c1ccnn1CC] | Cl | H |
| 2169 | 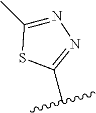 [SMILES: *c1nnc(C)s1] | CHF₂ | 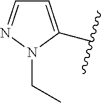 [SMILES: *c1ccnn1CC] | Cl | H |
| 2170 | 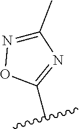 [SMILES: *c1nc(C)no1] | CHF₂ | 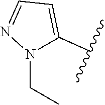 [SMILES: *c1ccnn1CC] | Cl | H |
| 2171 | 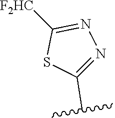 [SMILES: *c1nnc(C(F)F)s1] | CHF₂ | 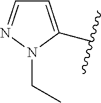 [SMILES: *c1ccnn1CC] | Cl | H |
| 2172 | 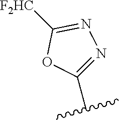 [SMILES: *c1nnc(C(F)F)o1] | CHF₂ | 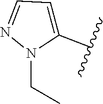 [SMILES: *c1ccnn1CC] | CI | H |
| 2173 | 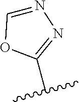 [SMILES: *c1nnco1] | CHF₂ | 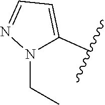 [SMILES: *c1ccnn1CC] | Cl | H |
| 2174 | 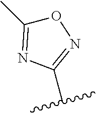 [SMILES: *c1noc(C)n1] | CHF₂ | 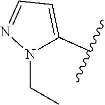 [SMILES: *c1ccnn1CC] | Cl | H |
| 2175 | 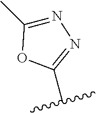 [SMILES: *c1nnc(C)o1] | CF₃ | 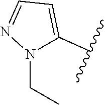 [SMILES: *c1ccnn1CC] | Cl | H |
| 2176 | 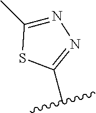 [SMILES: *c1nnc(C)s1] | CF₃ | 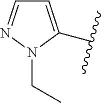 [SMILES: *c1ccnn1CC] | Cl | H |
| 2177 | 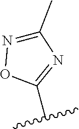 [SMILES: *c1nc(C)no1] | CF₃ | 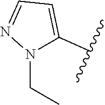 [SMILES: *c1ccnn1CC] | Cl | H |
| 2178 | 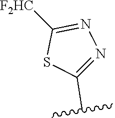 [SMILES: *c1nnc(C(F)F)s1] | CF₃ | 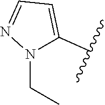 [SMILES: *c1ccnn1CC] | Cl | H |
| 2179 | 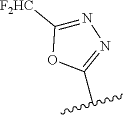 [SMILES: *c1nnc(C(F)F)o1] | CF₃ | 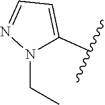 [SMILES: *c1ccnn1CC] | Cl | H |
| 2180 | 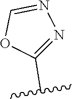 [SMILES: *c1nnco1] | CF₃ | 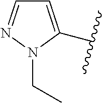 [SMILES: *c1ccnn1CC] | Cl | H |
| 2181 | 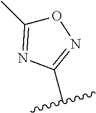 [SMILES: *c1noc(C)n1] | CF₃ | 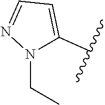 [SMILES: *c1ccnn1CC] | Cl | H |
| 2182 | 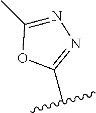 [SMILES: *c1nnc(C)o1] | CHF₂ | 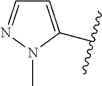 [SMILES: *c1ccnn1C] | Cl | H |
| 2183 | 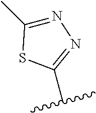 [SMILES: *c1nnc(C)s1] | CHF₂ | 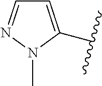 [SMILES: *c1ccnn1C] | Cl | H |
| 2184 | 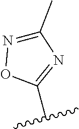 [SMILES: *c1nc(C)no1] | CHF₂ | 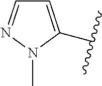 [SMILES: *c1ccnn1C] | Cl | H |
| 2185 | 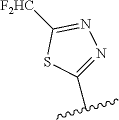 [SMILES: *c1nnc(C(F)F)s1] | CHF₂ | 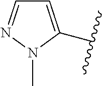 [SMILES: *c1ccnn1C] | Cl | H |
| 2186 | 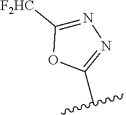 [SMILES: *c1nnc(C(F)F)o1] | CHF₂ | 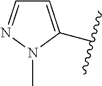 [SMILES: *c1ccnn1C] | Cl | H |
| 2187 | 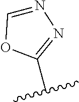 [SMILES: *c1nnco1] | CHF₂ | 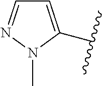 [SMILES: *c1ccnn1C] | Cl | H |
| 2188 | 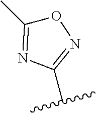 [SMILES: *c1noc(C)n1] | CHF₂ | 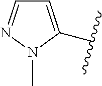 [SMILES: *c1ccnn1C] | Cl | H |
| 2189 | 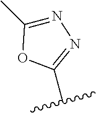 [SMILES: *c1nnc(C)o1] | CF₃ | 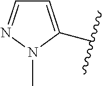 [SMILES: *c1ccnn1C] | Cl | H |
| 2190 | 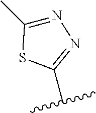 [SMILES: *c1nnc(C)s1] | CF₃ | 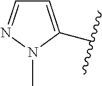 [SMILES: *c1ccnn1C] | Cl | H |
| 2191 | 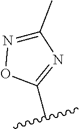 [SMILES: *c1nc(C)no1] | CF₃ | 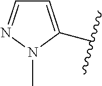 [SMILES: *c1ccnn1C] | Cl | H |
| 2192 | 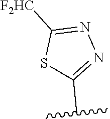 [SMILES: *c1nnc(C(F)F)s1] | CF₃ | 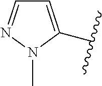 [SMILES: *c1ccnn1C] | Cl | H |
| 2193 | 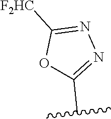 [SMILES: *c1nnc(C(F)F)o1] | CF₃ | 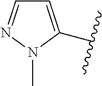 [SMILES: *c1ccnn1C] | Cl | H |
| 2194 | 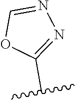 [SMILES: *c1nnco1] | CF₃ | 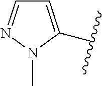 [SMILES: *c1ccnn1C] | Cl | H |
| 2195 | 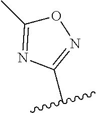 [SMILES: *c1noc(C)n1] | CF₃ | 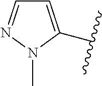 [SMILES: *c1ccnn1C] | Cl | H |
| 2196 | 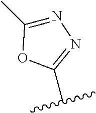 [SMILES: *c1nnc(C)o1] | CHF₂ | 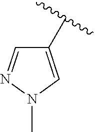 [SMILES: *c1cnn(C)c1] | Cl | H |
| 2197 | 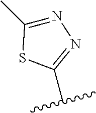 [SMILES: *c1nnc(C)s1] | CHF₂ | 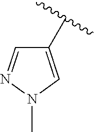 [SMILES: *c1cnn(C)c1] | Cl | H |
| 2198 | 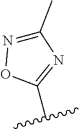 [SMILES: *c1nc(C)no1] | CHF₂ | 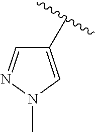 [SMILES: *c1cnn(C)c1] | Cl | H |
| 2199 | 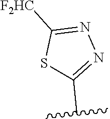 [SMILES: *c1nnc(C(F)F)s1] | CHF₂ | 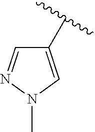 [SMILES: *c1cnn(C)c1] | Cl | H |
| 2200 | 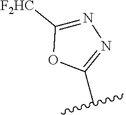 [SMILES: *c1nnc(C(F)F)o1] | CHF₂ | 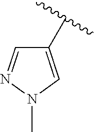 [SMILES: *c1cnn(C)c1] | Cl | H |
| 2201 | 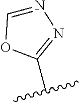 [SMILES: *c1nnco1] | CHF₂ | 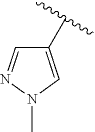 [SMILES: *c1cnn(C)c1] | Cl | H |
| 2202 | 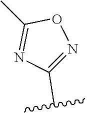 [SMILES: *c1noc(C)n1] | CHF₂ | 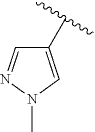 [SMILES: *c1cnn(C)c1] | Cl | H |
| 2203 | 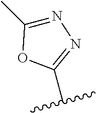 [SMILES: *c1nnc(C)o1] | CF₃ | 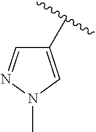 [SMILES: *c1cnn(C)c1] | Cl | H |
| 2204 | 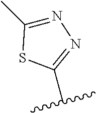 [SMILES: *c1nnc(C)s1] | CF₃ | 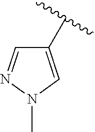 [SMILES: *c1cnn(C)c1] | Cl | H |
| 2205 | 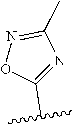 [SMILES: *c1nc(C)no1] | CF₃ | 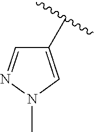 [SMILES: *c1cnn(C)c1] | Cl | H |
| 2206 | 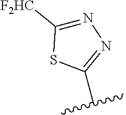 [SMILES: *c1nnc(C(F)F)s1] | CF₃ | 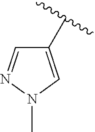 [SMILES: *c1cnn(C)c1] | Cl | H |
| 2207 | 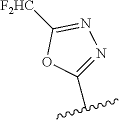 [SMILES: *c1nnc(C(F)F)o1] | CF₃ | 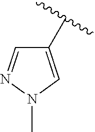 [SMILES: *c1cnn(C)c1] | Cl | H |
| 2208 | 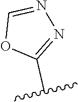 [SMILES: *c1nnco1] | CF₃ | 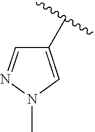 [SMILES: *c1cnn(C)c1] | Cl | H |
| 2209 | 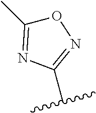 [SMILES: *c1noc(C)n1] | CF₃ | 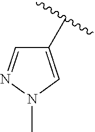 [SMILES: *c1cnn(C)c1] | Cl | H |
| 2210 | 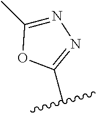 [SMILES: *c1nnc(C)o1] | H | 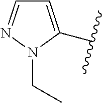 [SMILES: *c1ccnn1CC] | F | H |
| 2211 | 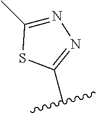 [SMILES: *c1nnc(C)s1] | H | 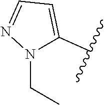 [SMILES: *c1ccnn1CC] | F | H |
| 2212 | 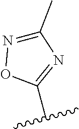 [SMILES: *c1nc(C)no1] | H | 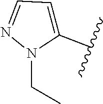 [SMILES: *c1ccnn1CC] | F | H |
| 2213 | 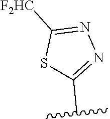 [SMILES: *c1nnc(C(F)F)s1] | H | 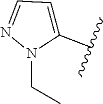 [SMILES: *c1ccnn1CC] | F | H |
| 2214 | 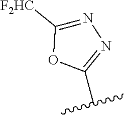 [SMILES: *c1nnc(C(F)F)o1] | H | 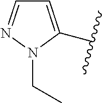 [SMILES: *c1ccnn1CC] | F | H |
| 2215 | 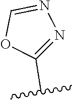 [SMILES: *c1nnco1] | H | 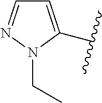 [SMILES: *c1ccnn1CC] | F | H |
| 2216 | 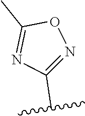 [SMILES: *c1noc(C)n1] | H | 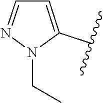 [SMILES: *c1ccnn1CC] | F | H |
| 2217 | 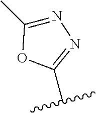 [SMILES: *c1nnc(C)o1] | CH₃ | 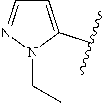 [SMILES: *c1ccnn1CC] | F | H |
| 2218 | 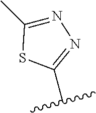 [SMILES: *c1nnc(C)s1] | CH₃ | 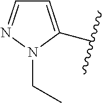 [SMILES: *c1ccnn1CC] | F | H |
| 2219 | 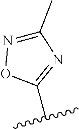 [SMILES: *c1nc(C)no1] | CH₃ | 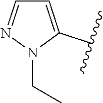 [SMILES: *c1ccnn1CC] | F | H |
| 2220 | 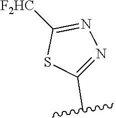 [SMILES: *c1nnc(C(F)F)s1] | CH₃ | 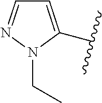 [SMILES: *c1ccnn1CC] | F | H |
| 2221 | 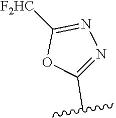 [SMILES: *c1nnc(C(F)F)o1] | CH₃ | 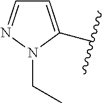 [SMILES: *c1ccnn1CC] | F | H |
| 2222 | 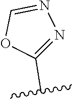 [SMILES: *c1nnco1] | CH₃ | 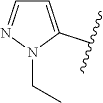 [SMILES: *c1ccnn1CC] | F | H |
| 2223 | 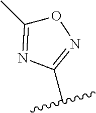 [SMILES: *c1noc(C)n1] | CH₃ | 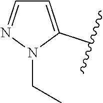 [SMILES: *c1ccnn1CC] | F | H |
| 2224 | 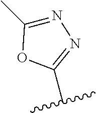 [SMILES: *c1nnc(C)o1] | H | 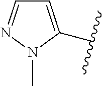 [SMILES: *c1ccnn1C] | F | H |
| 2225 | 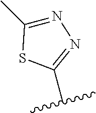 [SMILES: *c1nnc(C)s1] | H | 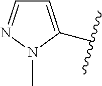 [SMILES: *c1ccnn1C] | F | H |
| 2226 | 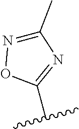 [SMILES: *c1nc(C)no1] | H | 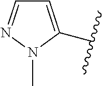 [SMILES: *c1ccnn1C] | F | H |
| 2227 | 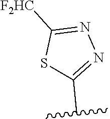 [SMILES: *c1nnc(C(F)F)s1] | H | 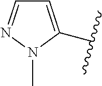 [SMILES: *c1ccnn1C] | F | H |
| 2228 | 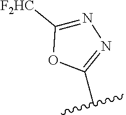 [SMILES: *c1nnc(C(F)F)o1] | H | 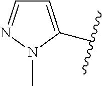 [SMILES: *c1ccnn1C] | F | H |
| 2229 | 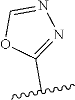 [SMILES: *c1nnco1] | H | 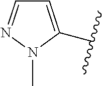 [SMILES: *c1ccnn1C] | F | H |
| 2230 | 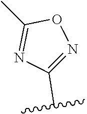 [SMILES: *c1noc(C)n1] | H | 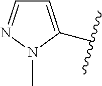 [SMILES: *c1ccnn1C] | F | H |
| 2231 | 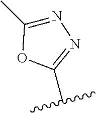 [SMILES: *c1nnc(C)o1] | CH₃ | 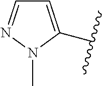 [SMILES: *c1ccnn1C] | F | H |
| 2232 | 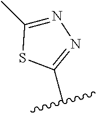 [SMILES: *c1nnc(C)s1] | CH₃ | 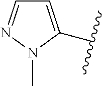 [SMILES: *c1ccnn1C] | F | H |
| 2233 | 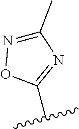 [SMILES: *c1nc(C)no1] | CH₃ | 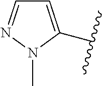 [SMILES: *c1ccnn1C] | F | H |
| 2234 | 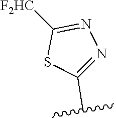 [SMILES: *c1nnc(C(F)F)s1] | CH₃ | 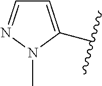 [SMILES: *c1ccnn1C] | F | H |
| 2235 | 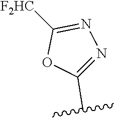 [SMILES: *c1nnc(C(F)F)o1] | CH₃ | 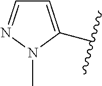 [SMILES: *c1ccnn1C] | F | H |
| 2236 | 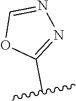 [SMILES: *c1nnco1] | CH₃ | 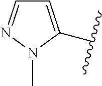 [SMILES: *c1ccnn1C] | F | H |
| 2237 | 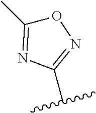 [SMILES: *c1noc(C)n1] | CH₃ | 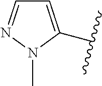 [SMILES: *c1ccnn1C] | F | H |
| 2238 | 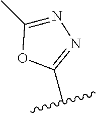 [SMILES: *c1nnc(C)o1] | H | 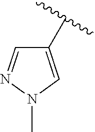 [SMILES: *c1cnn(C)c1] | F | H |
| 2239 | 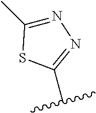 [SMILES: *c1nnc(C)s1] | H | 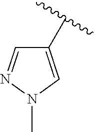 [SMILES: *c1cnn(C)c1] | F | H |
| 2240 | 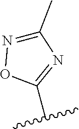 [SMILES: *c1nc(C)no1] | H | 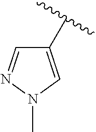 [SMILES: *c1cnn(C)c1] | F | H |
| 2241 | 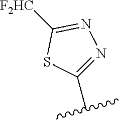 [SMILES: *c1nnc(C(F)F)s1] | H | 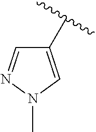 [SMILES: *c1cnn(C)c1] | F | H |
| 2242 | 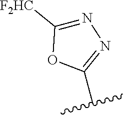 [SMILES: *c1nnc(C(F)F)o1] | H | 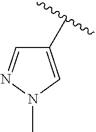 [SMILES: *c1cnn(C)c1] | F | H |
| 2243 | 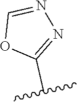 [SMILES: *c1nnco1] | H | 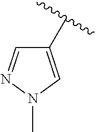 [SMILES: *c1cnn(C)c1] | F | H |
| 2244 | 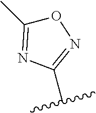 [SMILES: *c1noc(C)n1] | H | 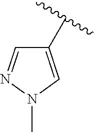 [SMILES: *c1cnn(C)c1] | F | H |
| 2245 | 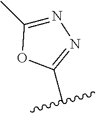 [SMILES: *c1nnc(C)o1] | CH₃ | 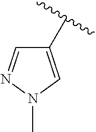 [SMILES: *c1cnn(C)c1] | F | H |
| 2246 | 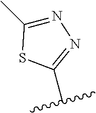 [SMILES: *c1nnc(C)s1] | CH₃ | 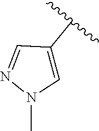 [SMILES: *c1cnn(C)c1] | F | H |
| 2247 | 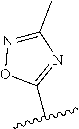 [SMILES: *c1nc(C)no1] | CH₃ | 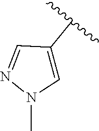 [SMILES: *c1cnn(C)c1] | F | H |
| 2248 | 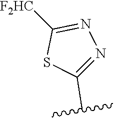 [SMILES: *c1nnc(C(F)F)s1] | CH₃ | 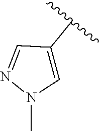 [SMILES: *c1cnn(C)c1] | F | H |
| 2249 | 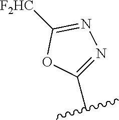 [SMILES: *c1nnc(C(F)F)o1] | CH₃ | 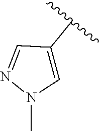 [SMILES: *c1cnn(C)c1] | F | H |
| 2250 | 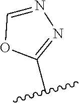 [SMILES: *c1nnco1] | CH₃ | 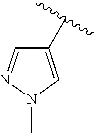 [SMILES: *c1cnn(C)c1] | F | H |
| 2251 | 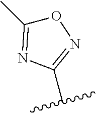 [SMILES: *c1noc(C)n1] | CH₃ | 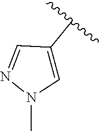 [SMILES: *c1cnn(C)c1] | F | H |
| 2252 | 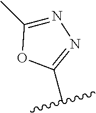 [SMILES: *c1nnc(C)o1] | H | 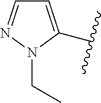 [SMILES: *c1ccnn1CC] | F | F |
| 2253 | 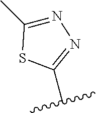 [SMILES: *c1nnc(C)s1] | H | 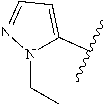 [SMILES: *c1ccnn1CC] | F | F |
| 2254 | 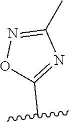 [SMILES: *c1nc(C)no1] | H | 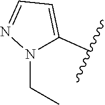 [SMILES: *c1ccnn1CC] | F | F |
| 2255 | 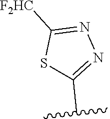 [SMILES: *c1nnc(C(F)F)s1] | H | 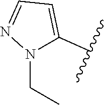 [SMILES: *c1ccnn1CC] | F | F |
| 2256 | 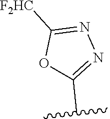 [SMILES: *c1nnc(C(F)F)o1] | H | 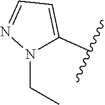 [SMILES: *c1ccnn1CC] | F | F |
| 2257 | 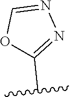 [SMILES: *c1nnco1] | H | 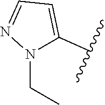 [SMILES: *c1ccnn1CC] | F | F |
| 2258 | 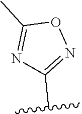 [SMILES: *c1noc(C)n1] | H | 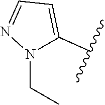 [SMILES: *c1ccnn1CC] | F | F |
| 2259 | 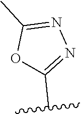 [SMILES: *c1nnc(C)o1] | CH₃ | 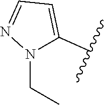 [SMILES: *c1ccnn1CC] | F | F |
| 2260 | 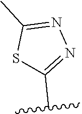 [SMILES: *c1nnc(C)s1] | CH₃ | 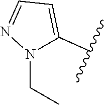 [SMILES: *c1ccnn1CC] | F | F |
| 2261 | 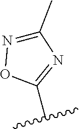 [SMILES: *c1nc(C)no1] | CH₃ | 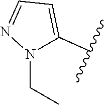 [SMILES: *c1ccnn1CC] | F | F |
| 2262 | 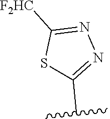 [SMILES: *c1nnc(C(F)F)s1] | CH₃ | 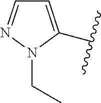 [SMILES: *c1ccnn1CC] | F | F |
| 2263 | 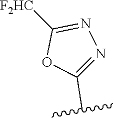 [SMILES: *c1nnc(C(F)F)o1] | CH₃ | 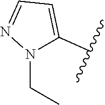 [SMILES: *c1ccnn1CC] | F | F |
| 2264 | 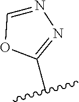 [SMILES: *c1nnco1] | CH₃ | 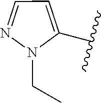 [SMILES: *c1ccnn1CC] | F | F |
| 2265 | 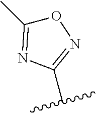 [SMILES: *c1noc(C)n1] | CH₃ | 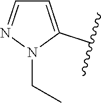 [SMILES: *c1ccnn1CC] | F | F |
| 2266 | 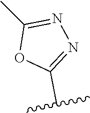 [SMILES: *c1nnc(C)o1] | H | 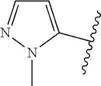 [SMILES: *c1ccnn1C] | F | F |
| 2267 | 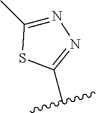 [SMILES: *c1nnc(C)s1] | H | 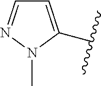 [SMILES: *c1ccnn1C] | F | F |
| 2268 | 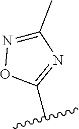 [SMILES: *c1nc(C)no1] | H | 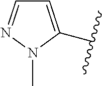 [SMILES: *c1ccnn1C] | F | F |
| 2269 | 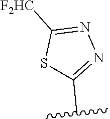 [SMILES: *c1nnc(C(F)F)s1] | H | 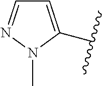 [SMILES: *c1ccnn1C] | F | F |
| 2270 | 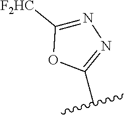 [SMILES: *c1nnc(C(F)F)o1] | H | 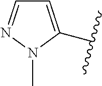 [SMILES: *c1ccnn1C] | F | F |
| 2271 | 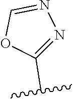 [SMILES: *c1nnco1] | H | 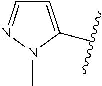 [SMILES: *c1ccnn1C] | F | F |
| 2272 | 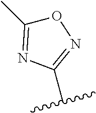 [SMILES: *c1noc(C)n1] | H | 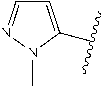 [SMILES: *c1ccnn1C] | F | F |
| 2273 | 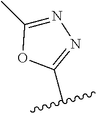 [SMILES: *c1nnc(C)o1] | CH₃ | 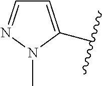 [SMILES: *c1ccnn1C] | F | F |
| 2274 | 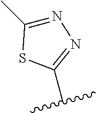 [SMILES: *c1nnc(C)s1] | CH₃ | 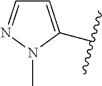 [SMILES: *c1ccnn1C] | F | F |
| 2275 | 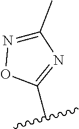 [SMILES: *c1nc(C)no1] | CH₃ | 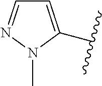 [SMILES: *c1ccnn1C] | F | F |
| 2276 | 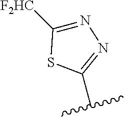 [SMILES: *c1nnc(C(F)F)s1] | CH₃ | 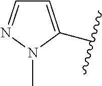 [SMILES: *c1ccnn1C] | F | F |
| 2277 | 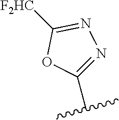 [SMILES: *c1nnc(C(F)F)o1] | CH₃ | 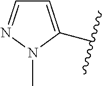 [SMILES: *c1ccnn1C] | F | F |
| 2278 | 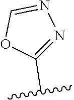 [SMILES: *c1nnco1] | CH₃ | 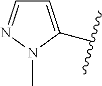 [SMILES: *c1ccnn1C] | F | F |
| 2279 | 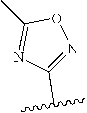 [SMILES: *c1noc(C)n1] | CH₃ | 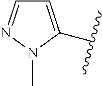 [SMILES: *c1ccnn1C] | F | F |
| 2280 | 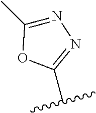 [SMILES: *c1nnc(C)o1] | H | 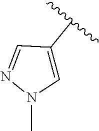 [SMILES: *c1cnn(C)c1] | F | F |
| 2281 | 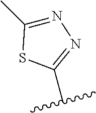 [SMILES: *c1nnc(C)s1] | H | 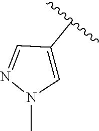 [SMILES: *c1cnn(C)c1] | F | F |
| 2282 | 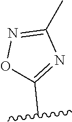 [SMILES: *c1nc(C)no1] | H | 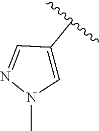 [SMILES: *c1cnn(C)c1] | F | F |
| 2283 | 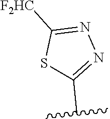 [SMILES: *c1nnc(C(F)F)s1] | H | 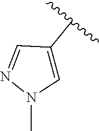 [SMILES: *c1cnn(C)c1] | F | F |
| 2284 | 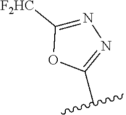 [SMILES: *c1nnc(C(F)F)o1] | H | 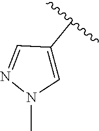 [SMILES: *c1cnn(C)c1] | F | F |
| 2285 | 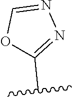 [SMILES: *c1nnco1] | H | 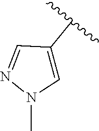 [SMILES: *c1cnn(C)c1] | F | F |
| 2286 | 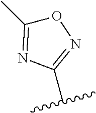 [SMILES: *c1noc(C)n1] | H | 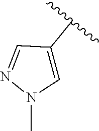 [SMILES: *c1cnn(C)c1] | F | F |
| 2287 | 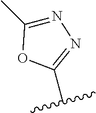 [SMILES: *c1nnc(C)o1] | CH₃ | 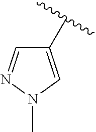 [SMILES: *c1cnn(C)c1] | F | F |
| 2288 | 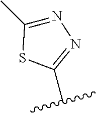 [SMILES: *c1nnc(C)s1] | CH₃ | 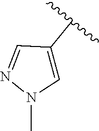 [SMILES: *c1cnn(C)c1] | F | F |
| 2289 | 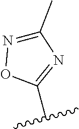 [SMILES: *c1nc(C)no1] | CH₃ | 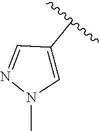 [SMILES: *c1cnn(C)c1] | F | F |
| 2290 | 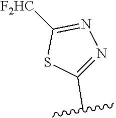 [SMILES: *c1nnc(C(F)F)s1] | CH₃ | 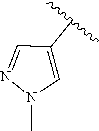 [SMILES: *c1cnn(C)c1] | F | F |
| 2291 | 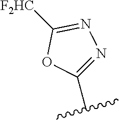 [SMILES: *c1nnc(C(F)F)o1] | CH₃ | 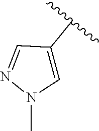 [SMILES: *c1cnn(C)c1] | F | F |
| 2292 | 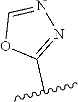 [SMILES: *c1nnco1] | CH₃ | 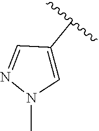 [SMILES: *c1cnn(C)c1] | F | F |
| 2293 | 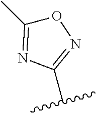 [SMILES: *c1noc(C)n1] | CH₃ | 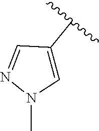 [SMILES: *c1cnn(C)c1] | F | F |
| 2294 | 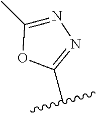 [SMILES: *c1nnc(C)o1] | H | 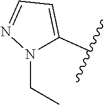 [SMILES: *c1ccnn1CC] | Cl | H |
| 2295 | 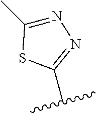 [SMILES: *c1nnc(C)s1] | H | 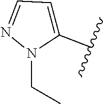 [SMILES: *c1ccnn1CC] | Cl | H |
| 2296 | 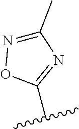 [SMILES: *c1nc(C)no1] | H | 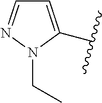 [SMILES: *c1ccnn1CC] | Cl | H |
| 2297 | 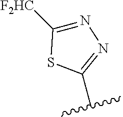 [SMILES: *c1nnc(C(F)F)s1] | H | 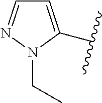 [SMILES: *c1ccnn1CC] | Cl | H |
| 2298 | 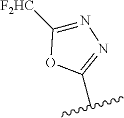 [SMILES: *c1nnc(C(F)F)o1] | H | 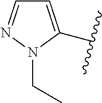 [SMILES: *c1ccnn1CC] | Cl | H |
| 2299 | 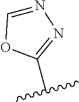 [SMILES: *c1nnco1] | H | 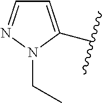 [SMILES: *c1ccnn1CC] | Cl | H |
| 2300 | 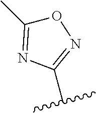 [SMILES: *c1noc(C)n1] | H | 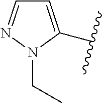 [SMILES: *c1ccnn1CC] | Cl | H |
| 2301 | 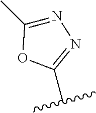 [SMILES: *c1nnc(C)o1] | CH₃ | 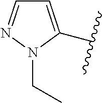 [SMILES: *c1ccnn1CC] | Cl | H |
| 2302 | 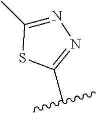 [SMILES: *c1nnc(C)s1] | CH₃ | 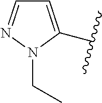 [SMILES: *c1ccnn1CC] | Cl | H |
| 2303 | 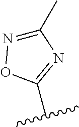 [SMILES: *c1nc(C)no1] | CH₃ | 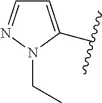 [SMILES: *c1ccnn1CC] | Cl | H |
| 2304 | 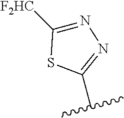 [SMILES: *c1nnc(C(F)F)s1] | CH₃ | 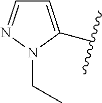 [SMILES: *c1ccnn1CC] | Cl | H |
| 2305 | 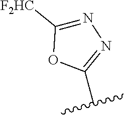 [SMILES: *c1nnc(C(F)F)o1] | CH₃ | 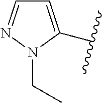 [SMILES: *c1ccnn1CC] | Cl | H |
| 2306 | 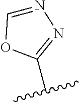 [SMILES: *c1nnco1] | CH₃ | 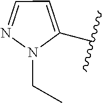 [SMILES: *c1ccnn1CC] | Cl | H |
| 2307 | 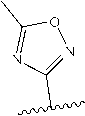 [SMILES: *c1noc(C)n1] | CH₃ | 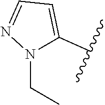 [SMILES: *c1ccnn1CC] | Cl | H |
| 2308 | 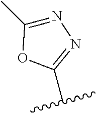 [SMILES: *c1nnc(C)o1] | H | 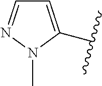 [SMILES: *c1ccnn1C] | Cl | H |
| 2309 | 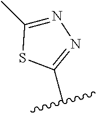 [SMILES: *c1nnc(C)s1] | H | 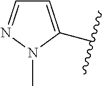 [SMILES: *c1ccnn1C] | Cl | H |
| 2310 | 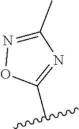 [SMILES: *c1nc(C)no1] | H | 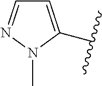 [SMILES: *c1ccnn1C] | Cl | H |
| 2311 | 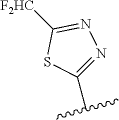 [SMILES: *c1nnc(C(F)F)s1] | H | 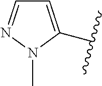 [SMILES: *c1ccnn1C] | Cl | H |
| 2312 | 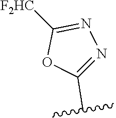 [SMILES: *c1nnc(C(F)F)o1] | H | 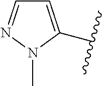 [SMILES: *c1ccnn1C] | Cl | H |
| 2313 | 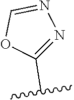 [SMILES: *c1nnco1] | H | 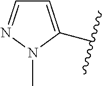 [SMILES: *c1ccnn1C] | Cl | H |
| 2314 | 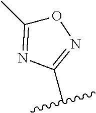 [SMILES: *c1noc(C)n1] | H | 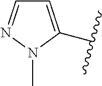 [SMILES: *c1ccnn1C] | Cl | H |
| 2315 | 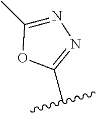 [SMILES: *c1nnc(C)o1] | CH₃ | 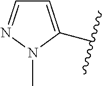 [SMILES: *c1ccnn1C] | Cl | H |
| 2316 | 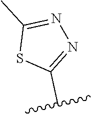 [SMILES: *c1nnc(C)s1] | CH₃ | 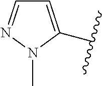 [SMILES: *c1ccnn1C] | Cl | H |
| 2317 | 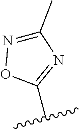 [SMILES: *c1nc(C)no1] | CH₃ | 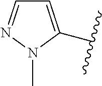 [SMILES: *c1ccnn1C] | Cl | H |
| 2317 | 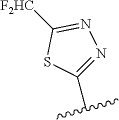 [SMILES: *c1nnc(C(F)F)s1] | CH₃ | 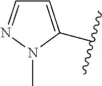 [SMILES: *c1ccnn1C] | Cl | H |
| 2319 | 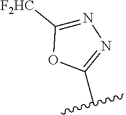 [SMILES: *c1nnc(C(F)F)o1] | CH₃ | 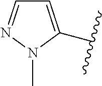 [SMILES: *c1ccnn1C] | Cl | H |
| 2320 | 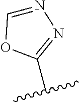 [SMILES: *c1nnco1] | CH₃ | 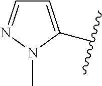 [SMILES: *c1ccnn1C] | Cl | H |
| 2321 | 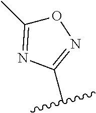 [SMILES: *c1noc(C)n1] | CH₃ | 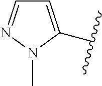 [SMILES: *c1ccnn1C] | Cl | H |
| 2322 | 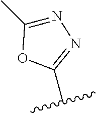 [SMILES: *c1nnc(C)o1] | H | 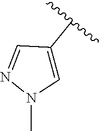 [SMILES: *c1cnn(C)c1] | Cl | H |
| 2323 | 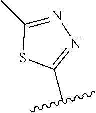 [SMILES: *c1nnc(C)s1] | H | 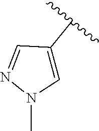 [SMILES: *c1cnn(C)c1] | Cl | H |
| 2324 | 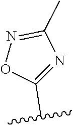 [SMILES: *c1nc(C)no1] | H | 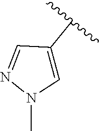 [SMILES: *c1cnn(C)c1] | Cl | H |
| 2325 | 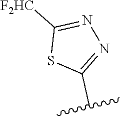 [SMILES: *c1nnc(C(F)F)s1] | H | 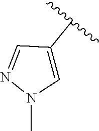 [SMILES: *c1cnn(C)c1] | Cl | H |
| 2326 | 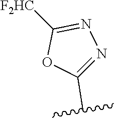 [SMILES: *c1nnc(C(F)F)o1] | H | 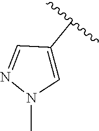 [SMILES: *c1cnn(C)c1] | Cl | H |
| 2327 | 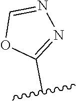 [SMILES: *c1nnco1] | H | 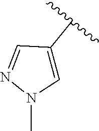 [SMILES: *c1cnn(C)c1] | Cl | H |
| 2328 | 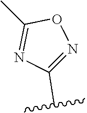 [SMILES: *c1noc(C)n1] | H | 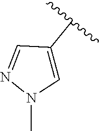 [SMILES: *c1cnn(C)c1] | Cl | H |
| 2329 | 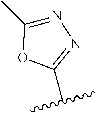 [SMILES: *c1nnc(C)o1] | CH₃ | 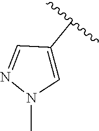 [SMILES: *c1cnn(C)c1] | Cl | H |
| 2330 | 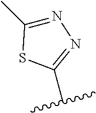 [SMILES: *c1nnc(C)s1] | CH₃ | 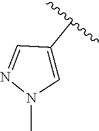 [SMILES: *c1cnn(C)c1] | Cl | H |
| 2331 | 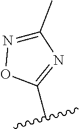 [SMILES: *c1nc(C)no1] | CH₃ | 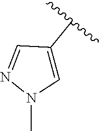 [SMILES: *c1cnn(C)c1] | Cl | H |
| 2334 | 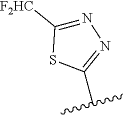 [SMILES: *c1nnc(C(F)F)s1] | CH₃ | 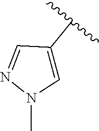 [SMILES: *c1cnn(C)c1] | Cl | H |
| 2335 | 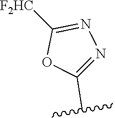 [SMILES: *c1nnc(C(F)F)o1] | CH₃ | 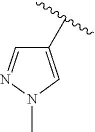 [SMILES: *c1cnn(C)c1] | Cl | H |
| 2336 | 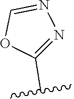 [SMILES: *c1nnco1] | CH₃ | 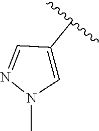 [SMILES: *c1cnn(C)c1] | Cl | H |
| 2337 | 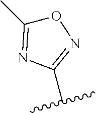 [SMILES: *c1noc(C)n1] | CH₃ | 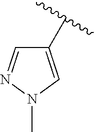 [SMILES: *c1cnn(C)c1] | Cl | H |

25. The compound of any one of claims 1-4, wherein the compound of formula (A), formula (I), or formula (II) is a compound having any one of the following formula, or a pharmaceutically acceptable salt, solvate, hydrate, cocrystal, or prodrug thereof:

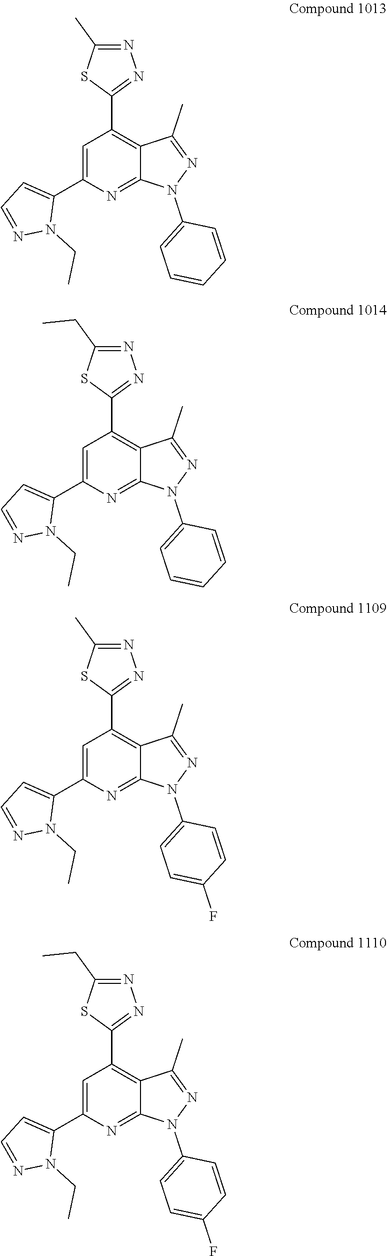

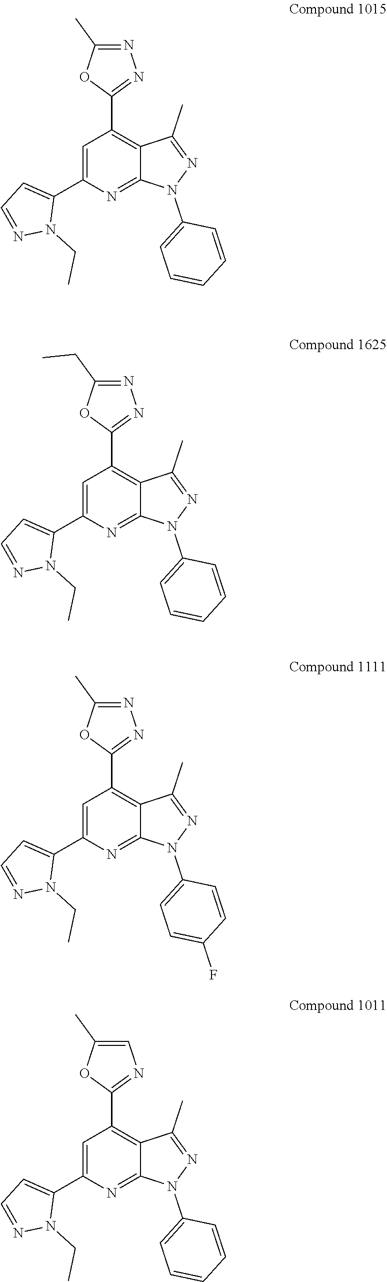 [SMILES: CCn1nccc1-c1cc(-c2nnc(C)o2)c2c(C)nn(-c3ccccc3)c2n1 ; CCc1nnc(-c2cc(-c3ccnn3CC)nc3c2c(C)nn3-c2ccccc2)o1 ; CCn1nccc1-c1cc(-c2nnc(C)o2)c2c(C)nn(-c3ccc(F)cc3)c2n1 ; CCn1nccc1-c1cc(-c2ncc(C)o2)c2c(C)nn(-c3ccccc3)c2n1]

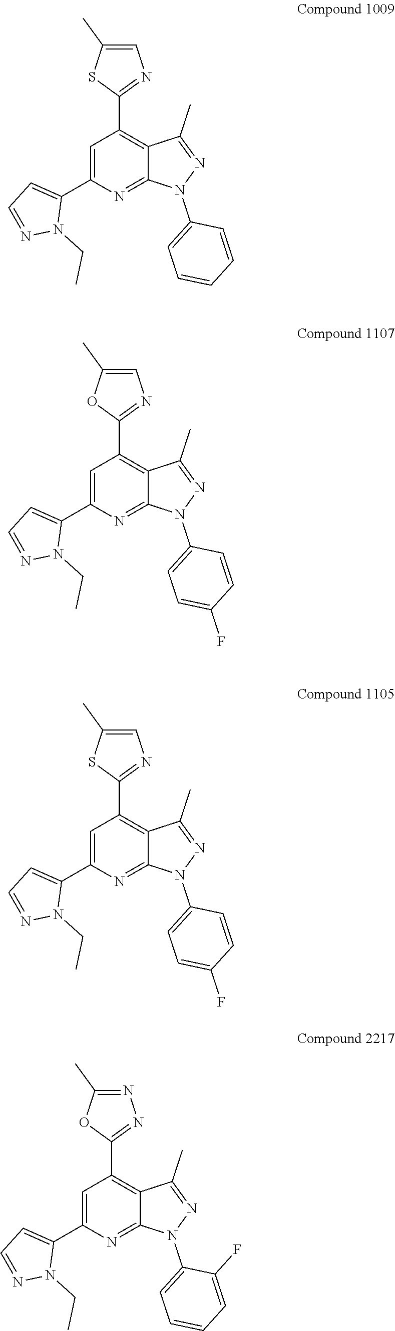 [SMILES: CCn1nccc1-c1cc(-c2ncc(C)s2)c2c(C)nn(-c3ccccc3)c2n1 ; CCn1nccc1-c1cc(-c2ncc(C)o2)c2c(C)nn(-c3ccc(F)cc3)c2n1 ; CCn1nccc1-c1cc(-c2ncc(C)s2)c2c(C)nn(-c3ccc(F)cc3)c2n1 ; CCn1nccc1-c1cc(-c2nnc(C)o2)c2c(C)nn(-c3ccccc3F)c2n1]

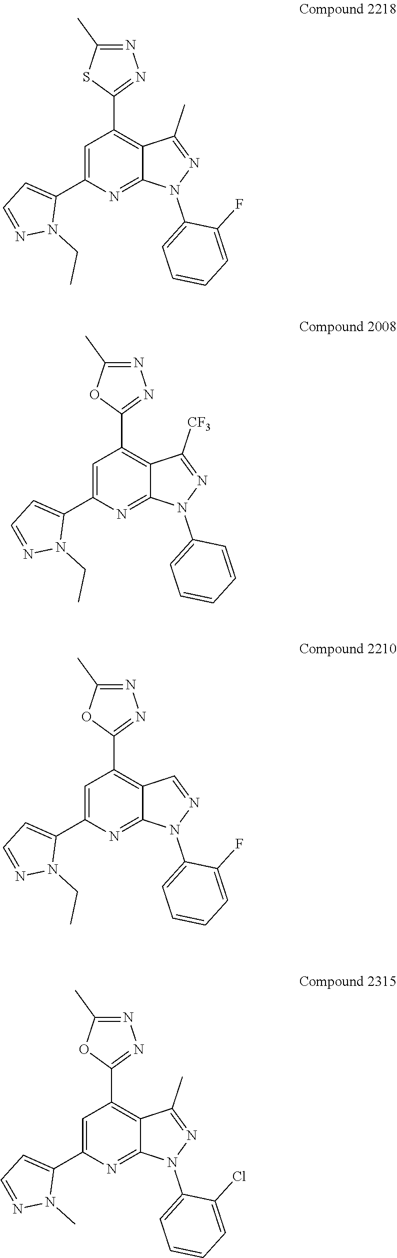 [SMILES: CCn1nccc1-c1cc(-c2nnc(C)s2)c2c(C)nn(-c3ccccc3F)c2n1 ; CCn1nccc1-c1cc(-c2nnc(C)o2)c2c(C(F)(F)F)nn(-c3ccccc3)c2n1 ; CCn1nccc1-c1cc(-c2nnc(C)o2)c2cnn(-c3ccccc3F)c2n1 ; Cc1nnc(-c2cc(-c3ccnn3C)nc3c2c(C)nn3-c2ccccc2Cl)o1]

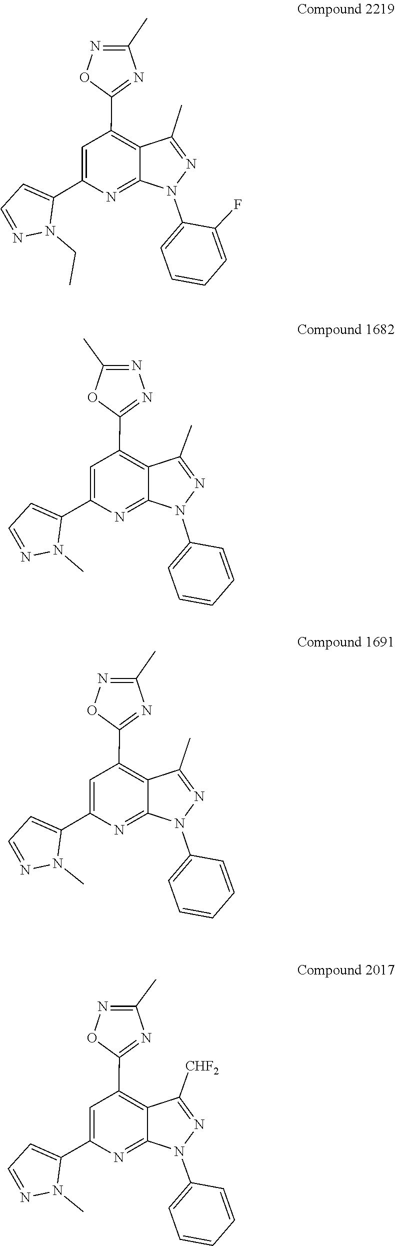 [SMILES: CCn1nccc1-c1cc(-c2nc(C)no2)c2c(C)nn(-c3ccccc3F)c2n1 ; Cc1nnc(-c2cc(-c3ccnn3C)nc3c2c(C)nn3-c2ccccc2)o1 ; Cc1noc(-c2cc(-c3ccnn3C)nc3c2c(C)nn3-c2ccccc2)n1 ; Cc1noc(-c2cc(-c3ccnn3C)nc3c2c(C(F)F)nn3-c2ccccc2)n1]

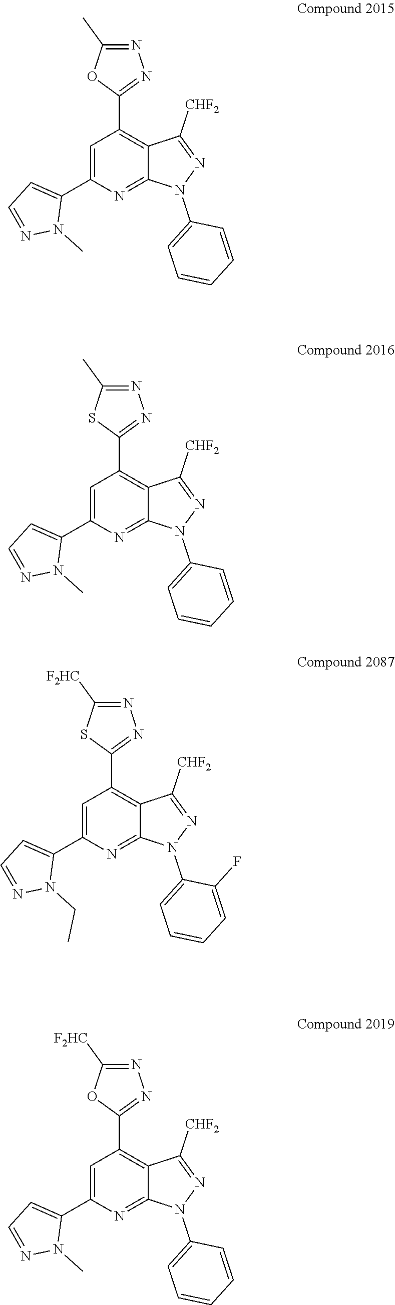 [SMILES: Cc1nnc(-c2cc(-c3ccnn3C)nc3c2c(C(F)F)nn3-c2ccccc2)o1 ; Cc1nnc(-c2cc(-c3ccnn3C)nc3c2c(C(F)F)nn3-c2ccccc2)s1 ; CCn1nccc1-c1cc(-c2nnc(C(F)F)s2)c2c(C(F)F)nn(-c3ccccc3F)c2n1 ; Cn1nccc1-c1cc(-c2nnc(C(F)F)o2)c2c(C(F)F)nn(-c3ccccc3)c2n1]

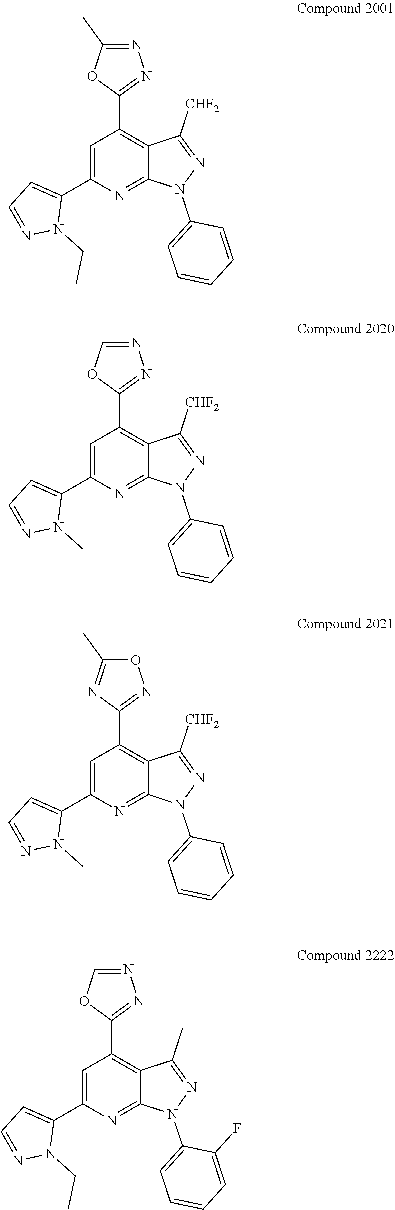 [SMILES: CCn1nccc1-c1cc(-c2nnc(C)o2)c2c(C(F)F)nn(-c3ccccc3)c2n1 ; Cn1nccc1-c1cc(-c2nnco2)c2c(C(F)F)nn(-c3ccccc3)c2n1 ; Cc1nc(-c2cc(-c3ccnn3C)nc3c2c(C(F)F)nn3-c2ccccc2)no1 ; CCn1nccc1-c1cc(-c2nnco2)c2c(C)nn(-c3ccccc3F)c2n1]

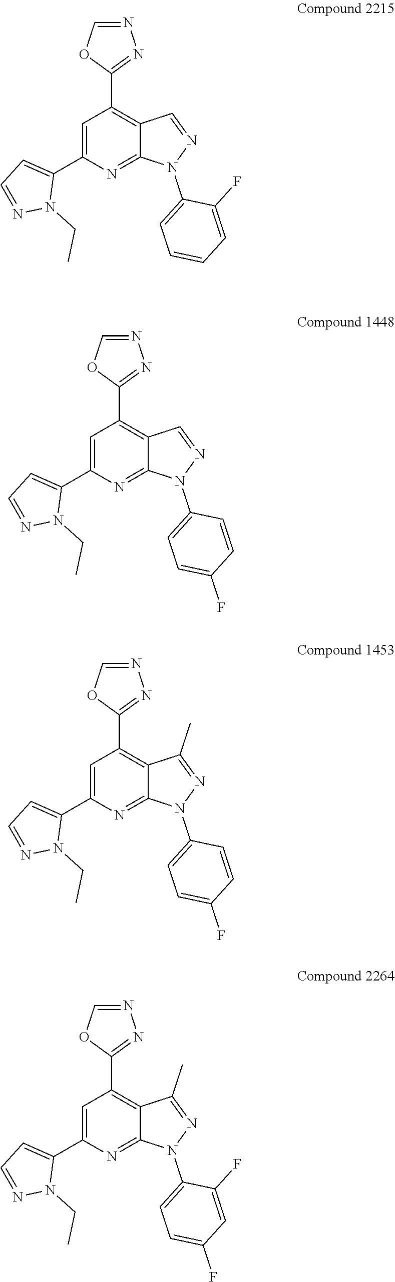 [SMILES: CCn1nccc1-c1cc(-c2nnco2)c2cnn(-c3ccccc3F)c2n1 ; CCn1nccc1-c1cc(-c2nnco2)c2cnn(-c3ccc(F)cc3)c2n1 ; CCn1nccc1-c1cc(-c2nnco2)c2c(C)nn(-c3ccc(F)cc3)c2n1 ; CCn1nccc1-c1cc(-c2nnco2)c2c(C)nn(-c3ccc(F)cc3F)c2n1]

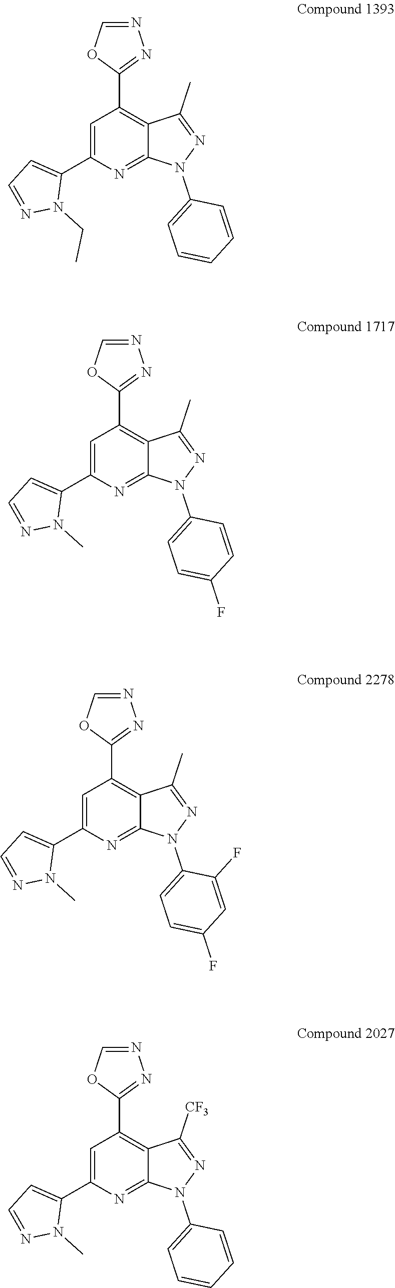 [SMILES: CCn1nccc1-c1cc(-c2nnco2)c2c(C)nn(-c3ccccc3)c2n1 ; Cc1nn(-c2ccc(F)cc2)c2nc(-c3ccnn3C)cc(-c3nnco3)c12 ; Cc1nn(-c2ccc(F)cc2F)c2nc(-c3ccnn3C)cc(-c3nnco3)c12 ; Cn1nccc1-c1cc(-c2nnco2)c2c(C(F)(F)F)nn(-c3ccccc3)c2n1]

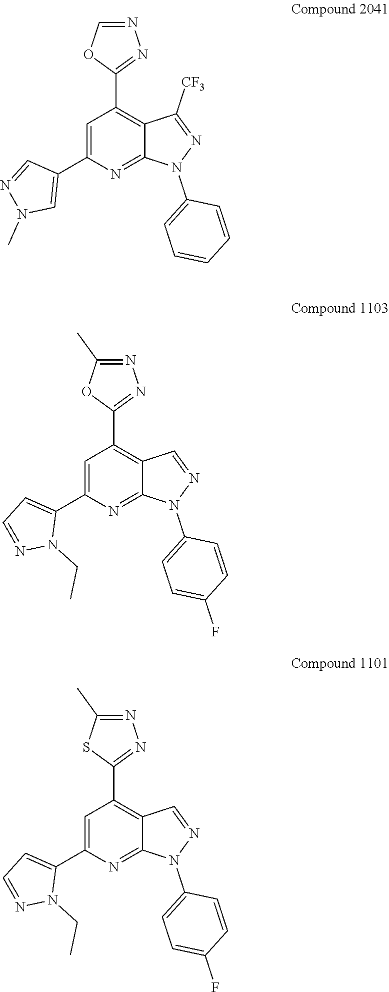

26. The compound of claim 1, or pharmaceutically acceptable salt, solvate, hydrate, cocrystal, or prodrug thereof, wherein:
R¹ is 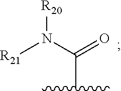
and
R₂₀ is selected from H, optionally substituted C₁-C₆ alkyl, and optionally substituted C₁-C₆ haloalkyl; R₂ is selected from optionally substituted C₁-C₆ alkyl and optionally substituted C₁-C₆ haloalkyl;
or R₂₀ and R₂₁ are joined to form a 4-6-membered heterocycle with the nitrogen to which they are each bound.

27. The compound of claim 26, or pharmaceutically acceptable salt, solvate, hydrate, cocrystal, or prodrug thereof, wherein R₂₀ is selected from H, methyl, ethyl, propyl, isopropyl, butyl, tert-butyl, CF₃, CHF₂, CH₂F, CH₂CF₃, CH₂CHF₂, and CH₂CH₂F; and R₂₁ is selected from methyl, ethyl, propyl, isopropyl, butyl, tert-butyl, CF₃, CHF₂, CH₂F, CH₂CF₃, CH₂CHF₂, and CH₂CH₂F;
or R₂₀ and R₂₁ are joined to form a 4-6-membered heterocycle with the nitrogen to which they are each bound.

28. The compound of claim 26 or 27, or pharmaceutically acceptable salt, solvate, hydrate, cocrystal, or prodrug thereof, wherein R₂₀ is selected from H, methyl, ethyl, propyl, tert-butyl, and CH₂CH₂F; and R₂₁ is selected from methyl, ethyl, propyl, tert-butyl, and CH₂CH₂F;
or R₂₀ and R₂₁ are joined to form a 4-6-membered heterocycle with the nitrogen to which they are each bound.

29. The compound of claim 1 or 26, or pharmaceutically acceptable salt, solvate, hydrate, cocrystal, or prodrug thereof, wherein the compound is of formula (III): 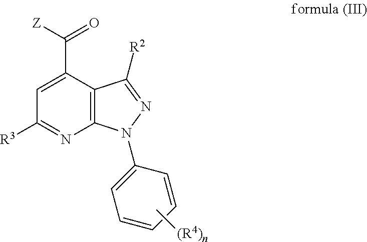
wherein in formula (III):
Z is selected from 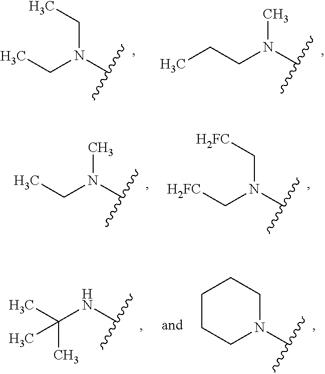
R³ is selected from 5-membered heterocycle, 5-membered heteroaryl, 6-membered heterocycle, and 6-membered heteroaryl;
R₂ and R⁴ are independently at each occurrence selected from hydrogen, optionally substituted alkyl, optionally substituted heteroalkyl, optionally substituted alkylheteroaryl, optionally substituted haloalkyl, optionally substituted alkenyl, optionally substituted alkynyl, optionally substituted cycloalkyl, optionally substituted heterocycloalkyl, optionally substituted alkylaryl, optionally substituted aryl, optionally substituted arylalkyl, optionally substituted heteroaryl, optionally substituted heteroarylalkyl, hydroxy, halo, cyano, trifluoromethyl, trifluoromethoxy, nitro, trimethylsilanyl, —OR^{a}, SR^{a}, —OC(O)—R^{a}, —SC(O)—R^{a}, —N(R^{a})₂, —C(O)R^{a}, —C(O)OR^{a}, —C(O)SR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a}, —S(O)ₜR^{a}, —S(O)ₜOR^{a}, —S(O)ₜN(R^{a})₂, and PO₃(R^{a})₂;
R^{a} is independently selected at each occurrence from hydrogen, optionally substituted alkyl, optionally substituted haloalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl; and
n is an integer selected from 0-5; and
t is 1 or 2.

30. The compound of any one of claims 26-29, wherein R₂ is selected from hydrogen, C₃-C₆ cycloalkyl, C₁-C₆ alkyl (e.g., C₁-C₄ straight chain alkyl, C₃-C₆ branched alkyl), —CF₃, —CH₂F, —CHF₂, and —C(R⁶R⁷)ₚX;
wherein, p is an integer from 1-3;
R⁶ and R⁷ are each independently selected from hydrogen, C₁-C₃ alkyl, and C₃-C₆ cycloalkyl;
X is selected from —OH, —CN, —OR^{a}, and NR⁹R¹⁰;
R⁸ is selected from C₁-C₃ alkyl (e.g., C₁-C₃ straight or branched alkyl), and C₃-C₆ cycloalkyl; and
R⁹ and R¹⁰ are each independently selected from hydrogen, C₁-C₃ alkyl, and C₃-C₆ cycloalkyl.

31. The compound of claim 30, wherein R₂ is selected from hydrogen, C₁-C₃ alkyl (e.g., methyl, ethyl, propyl, isopropyl), —CF₃, —CH₂F, and —CHF₂.

32. The compound of claim 30 or 31, wherein R₂ is selected from hydrogen and —CH₃.

33. The compound of any one of claims 26-32, wherein is R³ is a 5-membered heteroaryl ring, optionally selected from pyrrole, thiophene, furan, pyrazole, thiazole, isothiazole, oxazole, isoxazole, and imidazole.

34. The compound of claim 33, wherein R³ is selected from 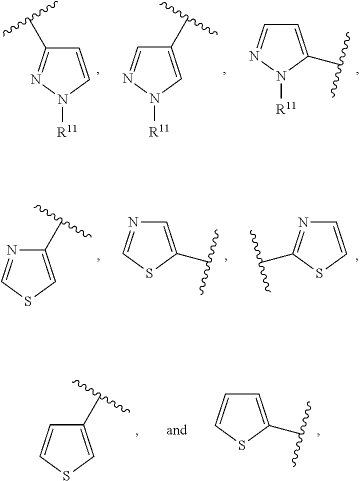
wherein R¹¹ is selected from hydrogen, C₁-C₃ alkyl (e.g., methyl, ethyl, propyl, isopropyl), C₃-C₆ cycloalkyl, CF₃, CH₂CF₃, CH₂CHF₂, and CH₂CH₂F.

35. The compound of claim 34, wherein R³ is selected from

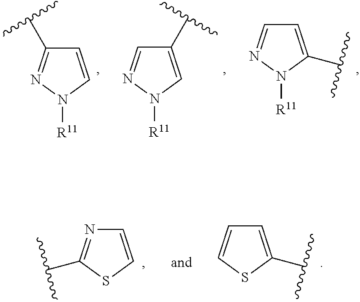

36. The compound of claim 34 or 35, wherein R¹¹ is selected from hydrogen, methyl, ethyl, propyl, isopropyl, —CF₃, and —CH₂CF₃.

37. The compound of any one of claims 26-36, wherein R³ is selected from

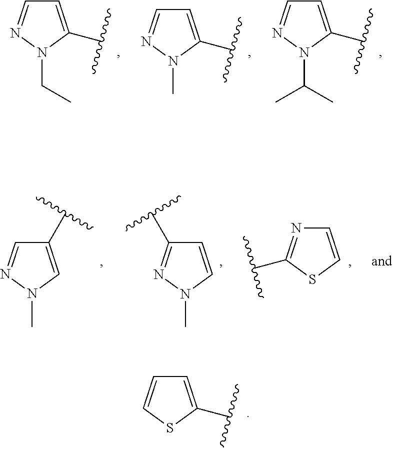

38. The compound of any one of claims 26-37, wherein R⁴ is selected from hydrogen, C₁-C₆ alkyl, and halo (e.g., F or Cl).

39. The compound of any one of claims 26-38, wherein n is 0 or 1.

40. The compound of any one of claims 26-39, wherein R⁴ is selected from hydrogen, 4-fluoro, 3-fluoro, 2-fluoro, 2-chloro, and 4-chloro.

41. The compound of any one of claims 26-40, wherein R⁴ is selected from hydrogen, 4-fluoro, 3-fluoro, 2-fluoro, and 4-chloro.

42. The compound of claim 29, wherein the compound of formula (III) is a compound having any one of formula 3001, 3006-3021, 3023-3031, or a pharmaceutically acceptable salt, solvate, hydrate, cocrystal, or prodrug thereof:

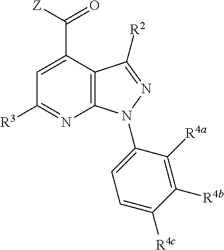

| Cpd. # | Z | R² | R³ | R^{4a} | R^{4b} | R^{4c} |
|---|---|---|---|---|---|---|
| 3001 | 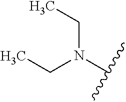 [SMILES: *N(CC)CC] | CH₃ | 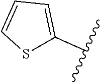 [SMILES: *c1cccs1] | H | H | H |
| 3006 | 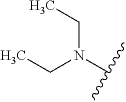 [SMILES: *N(CC)CC] | CH₃ | 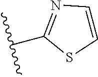 [SMILES: *c1nccs1] | H | H | H |
| 3007 | 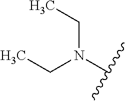 [SMILES: *N(CC)CC] | CH₃ | 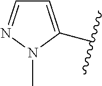 [SMILES: *c1ccnn1C] | H | H | H |
| 3008 | 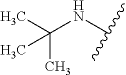 [SMILES: *NC(C)(C)C] | CH₃ | 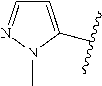 [SMILES: *c1ccnn1C] | H | H | H |
| 3009 | 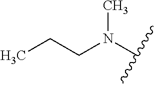 [SMILES: *N(C)CCC] | CH₃ | 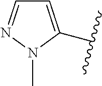 [SMILES: *c1ccnn1C] | H | H | H |
| 3010 | 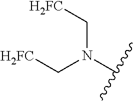 | CH₃ | 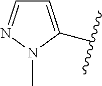 [SMILES: *c1ccnn1C] | H | H | H |
| 3011 | 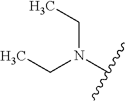 [SMILES: *N(CC)CC] | CH₃ | 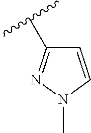 [SMILES: *c1ccn(C)n1] | H | H | H |
| 3012 | 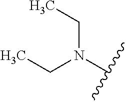 [SMILES: *N(CC)CC] | CH₃ | 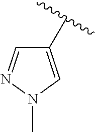 [SMILES: *c1cnn(C)c1] | H | H | H |
| 3013 | 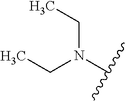 [SMILES: *N(CC)CC] | CH₃ | 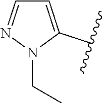 [SMILES: *c1ccnn1CC] | H | H | H |
| 3014 | 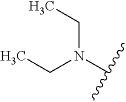 [SMILES: *N(CC)CC] | CH₃ | 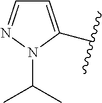 [SMILES: *c1ccnn1C(C)C] | H | H | H |
| 3015 | 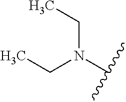 [SMILES: *N(CC)CC] | CH₃ | 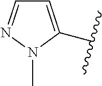 [SMILES: *c1ccnn1C] | H | H | F |
| 3016 | 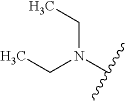 [SMILES: *N(CC)CC] | CH₃ | 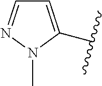 [SMILES: *c1ccnn1C] | H | F | H |
| 3017 | 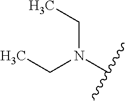 [SMILES: *N(CC)CC] | CH₃ | 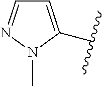 [SMILES: *c1ccnn1C] | H | H | Cl |
| 3018 | 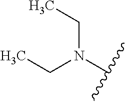 [SMILES: *N(CC)CC] | CH₃ | 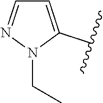 [SMILES: *c1ccnn1CC] | H | H | F |
| 3019 | 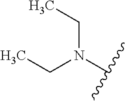 [SMILES: *N(CC)CC] | CH₃ | 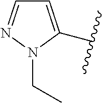 [SMILES: *c1ccnn1CC] | F | H | H |
| 3020 | 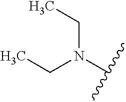 [SMILES: *N(CC)CC] | H | 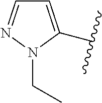 [SMILES: *c1ccnn1CC] | H | H | H |
| 3021 | 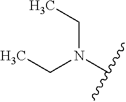 [SMILES: *N(CC)CC] | H | 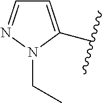 [SMILES: *c1ccnn1CC] | H | H | F |
| 3023 | 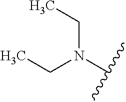 [SMILES: *N(CC)CC] | CH₃ | 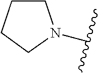 [SMILES: *N1CCCC1] | H | H | H |
| 3024 | 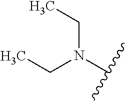 [SMILES: *N(CC)CC] | CH₃ | 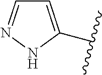 [SMILES: *c1ccn[nH]1] | H | H | H |
| 3025 | 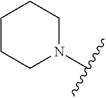 [SMILES: *N1CCCCC1] | CH₃ | 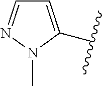 [SMILES: *c1ccnn1C] | H | H | H |
| 3026 | 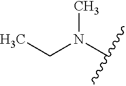 [SMILES: *N(C)CC] | CH₃ | 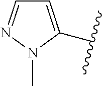 [SMILES: *c1ccnn1C] | H | H | H |
| 3027 | 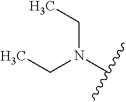 [SMILES: *N(CC)CC] | CH₃ | 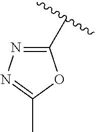 [SMILES: *c1nnc(C)o1] | H | H | H |
| 3028 | 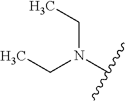 [SMILES: *N(CC)CC] | CH₃ | 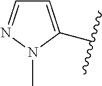 [SMILES: *c1ccnn1C] | Cl | H | H |
| 3029 | 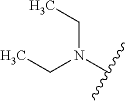 [SMILES: *N(CC)CC] | CF₃ | 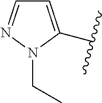 [SMILES: *c1ccnn1CC] | H | H | H |
| 3030 | 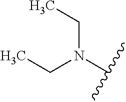 [SMILES: *N(CC)CC] | CHF₂ | 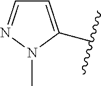 [SMILES: *c1ccnn1C] | H | H | H |
| 3031 | 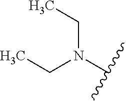 [SMILES: *N(CC)CC] | CH₃ | 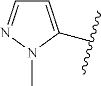 [SMILES: *c1ccnn1C] | H | H | H |

43. The compound of any one of claims 1 to 42, wherein the compound is a PDE11A4 inhibitor.

44. The compound of claim 43, wherein the PDE11A4 inhibitor is a PDE11A4 selective inhibitor.

45. A pharmaceutical composition comprising a compound of any one of claims 1 to 43, or a pharmaceutically acceptable salt, solvate, hydrate, cocrystal, or prodrug thereof, and a physiologically compatible carrier medium.

46. A pharmaceutical composition comprising a compound of any one of claims 1 to 43, or a pharmaceutically acceptable salt, solvate, hydrate, cocrystal, or prodrug thereof, and a physiologically compatible carrier medium, wherein the amount of the compound in the composition is a therapeutically effective amount for the treatment or prevention of a disease or disorder alleviated by inhibiting PDE11A4 activity in a patient in need thereof.

47. The pharmaceutical composition of claim 45 or 46, wherein the disease or disorder is associated with cognitive decline.

48. The pharmaceutical composition of claim 47, wherein the disease or disorder is selected from dementia; Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) including age-related cognitive decline of associative long-term memories (aLTMs), dementia associated with traumatic brain injury, preclinical Alzheimer's Disease (PCAD); Cognitive Impairment No Dementia (CIND); and cognitive decline associated with spatial memory, other depression-related behaviors, additional anxiety-related behaviors, sensorimotor gating, and social behaviors.

49. A method of treating or preventing a disease or disorder alleviated by inhibiting PDE11A4 activity in a patient in need thereof, comprising administering to the patient a therapeutically effective amount of a compound of any one of claims 1 to 43.

50. A method of treating or preventing a disease or disorder alleviated by inhibiting PDE11A4 activity in a patient in need thereof, comprising administering to the patient a therapeutically effective amount of a pharmaceutical composition of any one of claims 45-48.

51. The method of any one of claims 49 or 50, wherein the compound is administered in a dosage unit form.

52. The method of claim 51, wherein the dosage unit comprises a physiologically compatible carrier medium.

53. The method of any one of claims 49-52, wherein the disease or disorder is associated with cognitive decline.

54. The method of claim 53, wherein the disease or disorder is selected from dementia, Alzheimer's Disease (AD) including mild Alzheimer's disease and early-onset Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD) (including age-related cognitive decline of associative long-term memories (aLTMs), dementia associated with traumatic brain injury, preclinical Alzheimer's Disease (PCAD); Cognitive Impairment No Dementia (CIND), and cognitive decline associate with spatial memory, other depression-related behaviors, additional anxiety-related behaviors, sensorimotor gating, and social behaviors.
